SEQUENCE LISTING (128 sequences)

SEQ ID NO: 1 - DNA, 317 bp, synthetic construct
  source 1..317: mol_type = other DNA
caccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcggcggccgccacc

SEQ ID NO: 2 - DNA, 461 bp, synthetic construct
  source 1..461: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcggcggccgccacc

SEQ ID NO: 3 - DNA, 264 bp, synthetic construct
  source 1..264: mol_type = other DNA
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcggccgccacc

SEQ ID NO: 4 - DNA, 314 bp, synthetic construct
  source 1..314: mol_type = other DNA
caccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaagcggccgccacc

SEQ ID NO: 5 - DNA, 620 bp, synthetic construct
  source 1..620: mol_type = other DNA
gaattttaataaatacctctctgtccttctcctagcttcaccctttgtttgttgaaaggagatactttagtgcagtattgcaacagcttatgtagacatgcaaccttgaggtcacagacctatgagggattgagacaaaggcaaatttagcaaatattttccagagtactctgctgcaataacgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 6 - DNA, 23 bp, synthetic construct
  source 1..23: mol_type = other DNA
tccataaagtaggaaacactaca

SEQ ID NO: 7 - DNA, 618 bp, synthetic construct
  source 1..618: mol_type = other DNA
gaattttaataaatacctctctgtccttctcctagcttcaccctttgtttgttgaaaggagatactttagtgcagtattgcaacagcttatgtagacatgcaaccttgaggtcacagacctatgagggattgagacaaaggcaaatttagcaaatattttccagagtactctgctgcaataaccgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 8 - DNA, 564 bp, synthetic construct
  source 1..564: mol_type = other DNA
gagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 9 - DNA, 563 bp, synthetic construct
  source 1..563: mol_type = other DNA
gagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaag

SEQ ID NO: 10 - DNA, 581 bp, synthetic construct
  source 1..581: mol_type = other DNA
gagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaactcctaaaaaaccgccacc

SEQ ID NO: 11 - DNA, 586 bp, synthetic construct
  source 1..586: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 12 - DNA, 603 bp, synthetic construct
  source 1..603: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaactcctaaaaaaccgccacc

SEQ ID NO: 13 - DNA, 611 bp, synthetic construct
  source 1..611: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaactcctaaaaaaccgccaagatggcggc

SEQ ID NO: 14 - DNA, 585 bp, synthetic construct
  source 1..585: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaag

SEQ ID NO: 15 - DNA, 581 bp, synthetic construct
  source 1..581: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggaccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaagaggtaagggtttaagggatggttggttggtggggtattaatgtttaattacctggagcacctgcctgaaatcactttttttcaggttgggcggccgccacc

SEQ ID NO: 16 - DNA, 567 bp, synthetic construct
  source 1..567: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacagggcggccgccacc

SEQ ID NO: 17 - DNA, 489 bp, synthetic construct
  source 1..489: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgggtacccgggggcgcgccagtcaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcggcggccgccacc

SEQ ID NO: 18 - DNA, 522 bp, synthetic construct
  source 1..522: mol_type = other DNA
tcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcaccataaaagaagccgccctagccacgtcccctcgcagttcggcggtcccgcgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccggatccgccacc

SEQ ID NO: 19 - DNA, 536 bp, synthetic construct
  source 1..536: mol_type = other DNA
tcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccggatccgccacc

SEQ ID NO: 20 - DNA, 537 bp, synthetic construct
  source 1..537: mol_type = other DNA
agccaatgaaatacaaagatgagtctagttaataatctacaattattggttaaagaagtatattagtgctaatttccctccgtttgtcctagcttttctcgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccacc

SEQ ID NO: 21 - DNA, 761 bp, synthetic construct
  source 1..761: mol_type = other DNA
gagttgctggtgcttccccaggctggagattgagttaatattaacaggcccaaggcgatgtgggcttgtgcaatcataggcccggcctcgccaggtcacctgaggagttaatgaatacatatctcctgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgaggtcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccaccaaccaaccggatccgccacc

SEQ ID NO: 22 - DNA, 1017 bp, synthetic construct
  source 1..1017: mol_type = other DNA
tcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctcctgggcaggttggtatcaaggttacaagacaggtttaaggagaccaatagaaactgggcatgtggagacaggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtagaagactcttgggtttctgataggcactgactctctctgcctattggtctattttcccacccttaggctgcttcttgccaaccaacc

SEQ ID NO: 23 - DNA, 868 bp, synthetic construct
  source 1..868: mol_type = other DNA
gagttgctggtgcttccccaggctggagattgagttaatattaacaggcccaaggcgatgtgggcttgtgcaatcataggcccggcctcgccaggtcacctgaggagttaatgaatacatatctcctgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgaggtcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccacctccgaccgccctccgatttcctctccgcttgcaacctccgggaccatcttctcggccatctcctgcttctgggacctgccagcaccgtttttgtggttagctcctccaaccaaccggatccgccacc

SEQ ID NO: 24 - DNA, 255 bp, synthetic construct
  source 1..255: mol_type = other DNA
gttaatcattaagtcgttaatttttgtggcccttgcgatgtttgctctggttaataatctcaggacaaacagaggttaataattttccagatctctctgagcaatagtataaaaggccagcagcagcctgaccacatctcatccttgagccaagaggtaagggtttaagggatggttggttggtggggtattaatgtttaattacctggagcacctgcctgaaatcactttttttcaggttggggatccgccacc

SEQ ID NO: 25 - DNA, 283 bp, synthetic construct
  source 1..283: mol_type = other DNA
gttaatcattaagtcgttaatttttgtggcccttgcgatgtttgctctggttaataatctcaggacaaacagaggttaataattttccagatctctctgagcaatagtataaaaggccagcagcagcctgaccacatctcatccttgatccacaagctcgagccgagtggtcgtgagccaagaggtaagggtttaagggatggttggttggtggggtattaatgtttaattacctggagcacctgcctgaaatcactttttttcaggttggggatccgccacc

SEQ ID NO: 26 - DNA, 593 bp, synthetic construct
  source 1..593: mol_type = other DNA
gagttgctggtgcttccccaggctggagattgagttaatattaacaggcccaaggcgatgtgggcttgtgcaatcataggcccggcctcgccaggtcacctgaggagttaatgaatacatatctcctgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggttaatcattaagtcgttaatttttgtggcccttgcgatgtttgctctggttaataatctcaggacaaacagaggttaataattttccagatctctctgagcaatagtataaaaggccagcagcagcctgaccacatctcatccttgagccaagaggtaagggtttaagggatggttggttggtggggtattaatgtttaattacctggagcacctgcctgaaatcactttttttcaggttggggatccgccacc

SEQ ID NO: 27 - DNA, 621 bp, synthetic construct
  source 1..621: mol_type = other DNA
gagttgctggtgcttccccaggctggagattgagttaatattaacaggcccaaggcgatgtgggcttgtgcaatcataggcccggcctcgccaggtcacctgaggagttaatgaatacatatctcctgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggttaatcattaagtcgttaatttttgtggcccttgcgatgtttgctctggttaataatctcaggacaaacagaggttaataattttccagatctctctgagcaatagtataaaaggccagcagcagcctgaccacatctcatcctgatccacaagctcgagccgagtggtcgttgagccaagaggtaagggtttaagggatggttggttggtggggtattaatgtttaattacctggagcacctgcctgaaatcactttttttcaggttggggatccgccacc

SEQ ID NO: 28 - DNA, 548 bp, synthetic construct
  source 1..548: mol_type = other DNA
gagttaataattaccagcgcgggccaaataaataatccgcgaggggcagctggactttggactccgcgcgctggtaattattaacctccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcggcggccgccacc

SEQ ID NO: 29 - DNA, 633 bp, synthetic construct
  source 1..633: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgggtacccgggggcgcgccagtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcggcggccgccacc

SEQ ID NO: 30 - DNA, 594 bp, synthetic construct
  source 1..594: mol_type = other DNA
gagttaataattaccagcgcgggccaaataaataatccgcgccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaagaggtaagggtttaagggatggttggttggtggggtattaatgtttaattacctggagcacctgcctgaaatcactttttttcaggttgggcggccgccacc

SEQ ID NO: 31 - DNA, 725 bp, synthetic construct
  source 1..725: mol_type = other DNA
gagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaagaggtaagggtttaagggatggttggttggtggggtattaatgtttaattacctggagcacctgcctgaaatcactttttttcaggttgggcggccgccacc

SEQ ID NO: 32 - DNA, 561 bp, synthetic construct
  source 1..561: mol_type = other DNA
gagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgcggccgccacc

SEQ ID NO: 33 - DNA, 562 bp, synthetic construct
  source 1..562: mol_type = other DNA
gagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgcgcggccgcaag

SEQ ID NO: 34 - DNA, 4398 bp, Homo sapiens
  source 1..4398: mol_type = unassigned DNA
atgcctgaacaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcaggatcttgggcatgacttgccagtcatgtgtgaagtccattgaggacaggatttccaatttgaaaggcatcatcagcatgaaggtttccctggaacaaggcagtgccactgtgaaatatgtgccatcggttgtgtgcctgcaacaggtttgccatcaaattggggacatgggcttcgaggccagcattgcagaaggaaaggcagcctcctggccctcaaggtccttgcctgcccaggaggctgtggtcaagctccgggtggagggcatgacctgccagtcctgtgtcagctccattgaaggcaaggtccggaaactgcaaggagtagtgagagtcaaagtctcactcagcaaccaagaggccgtcatcacttatcagccttatctcattcagcccgaagacctcagggaccatgtaaatgacatgggatttgaagctgccatcaagagcaaagtggctcccttaagcctgggaccaattgatattgagcggttacaaagcactaacccaaagagacctttatcttctgctaaccagaattttaataattctgagaccttggggcaccaaggaagccatgtggtcaccctccaactgagaatagatggaatgcattgtaagtcttgcgtcttgaatattgaagaaaatattggccagctcctaggggttcaaagtattcaagtctccttggagaacaaaactgcccaagtaaagtatgacccttcttgtaccagcccagtggctctgcagagggctatcgaggcacttccacctgggaattttaaagtttctcttcctgatggagccgaagggagtgggacagatcacaggtcttccagttctcattcccctggctccccaccgagaaaccaggtccagggcacatgcagtaccactctgattgccattgccggcatgacctgtgcatcctgtgtccattccattgaaggcatgatctcccaactggaaggggtgcagcaaatatcggtgtctttggccgaagggactgcaacagttctttataatcccgctgtaattagcccagaagaactcagagctgctatagaagacatgggatttgaggcttcagtcgtttctgaaagctgttctactaaccctcttggaaaccacagtgctgggaattccatggtgcaaactacagatggtacacctacatctctgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcagggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagaaatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagccccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 35 - DNA, 3312 bp, synthetic construct
  source 1..3312: mol_type = other DNA
atggcggcgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggcccctgatggagccgaagggagtgggacagatcacaggtcttccagttctcattcccctggctccccaccgagaaaccaggtccagggcacatgcagtaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtcttttcttgtgagtctgtttttctgtattcctgtaatggggctgatgatatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctccagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 36 - DNA, 3495 bp, synthetic construct
  source 1..3495: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccactctgattgccattgccggcatgacctgtgcatcctgtgtccattccattgaaggcatgatctcccaactggaaggggtgcagcaaatatcggtgtctttggccgaagggactgcaacagttctttataatccctctgtaattagcccagaagaactcagagctgctatagaagacatgggatttgaggcttcagtcgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 37 - DNA, 3495 bp, synthetic construct
  source 1..3495: mol_type = other DNA
atgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccaggtggcgaccaccttaatcgccatcgcaggcatgacctgcgcatcttgtgtgcatagcattgaaggcatgattagccagctggagggcgtgcagcagatctcagtctccctggctgaggggaccgccactgtcttgtacaatccgtctgttatttccccggaggagctgagagctgccattgaggacatgggatttgaagcatcggtggtgtctgagtcatgctccaccaatcccctgggaaatcactcagctggcaattcaatggttcagaccaccgatgggaccccaaccagcgtgcaggaagtggcaccccataccggcagactgcctgccaatcatgctcctgatatcctcgccaagagcccccagagcacccgcgcggtggcaccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaa

SEQ ID NO: 38 - DNA, 3495 bp, synthetic construct
  source 1..3495: mol_type = other DNA
atgcctgaacaagaaagacagatcacagcaagagaaggagcatctaggaagatcctgagcaagctctccctgcccacaagggcctgggagcctgccatgaagaagtcttttgcctttgataatgtgggttatgaaggagggctggatggcctgggccccagcagccaggtggcaaccaccctgattgccattgcaggcatgacctgtgcatcttgtgtgcatagcattgaaggcatgattagccagctggagggagtgcagcagatctctgtctccctggctgaggggacagccactgtcctgtacaatccctctgttatttcccctgaggagctgagagctgccattgaggacatgggatttgaagcatctgtggtgtctgagtcctgctccaccaatcccctgggaaatcactctgctggcaattctatggttcagaccacagatgggaccccaacctctgtgcaggaagtggcaccccatactggcagactgcctgccaatcatgctcctgatatcctggccaagagcccccagagcaccagggctgtggcaccccagaagtgtttcctgcagatcaagggcatgacctgtgccagctgtgttagcaatattgaaagaaacctccaaaaagaggctggagttctgtctgttctggtggctctgatggctgggaaagcagagatcaagtatgatccagaggtgattcagccactggagattgcccagttcatccaggatctgggctttgaggcagctgtgatggaggattatgctggctctgatggcaacattgaactgaccatcacaggcatgacctgtgcttcctgtgtgcataatattgagagcaaactgaccagaaccaatggaatcacctatgcctctgttgccctggcaacctctaaggctctggtgaagtttgaccctgaaatcattggacctagagacatcatcaaaattattgaagagattggtttccatgccagcctggctcagagaaatcccaatgcacaccatctggaccataagatggaaatcaaacagtggaagaagagcttcctgtgtagcctggtctttgggattcctgtcatggcactgatgatttacatgctcattccctccaatgagccccaccagtccatggtgctggaccacaacatcatccctgggctgtctatcctcaacctgatcttctttattctgtgcacatttgtccagctcctgggaggctggtatttctatgttcaggcctacaaatccctgaggcatagatctgccaacatggatgttctgattgtgctggctacatccattgcctatgtgtactccctggtgatcctggtggttgctgtggctgaaaaggctgagagaagccctgtcaccttctttgacaccccccccatgctctttgtgtttattgcactgggtaggtggctggagcatctggccaagtccaagacatctgaggctctggccaagctgatgtctctccaggccactgaggccactgtggtgactctgggtgaagacaatctgatcattagagaggaacaggtgcctatggagctggtgcagagaggagatattgtcaaggtggtgccaggaggcaagttcccagtggatggaaaagtgctggagggcaataccatggctgatgaatccctcatcactggggaggcaatgcctgtgaccaagaaacctggaagcactgtcattgcaggcagcatcaatgcccatgggtctgtcctgatcaaggccacccatgtgggcaatgatacaactctggcccagattgtcaagctggtggaagaggctcagatgtctaaagctccaatccagcaactggctgacagattctctggctactttgtcccctttatcatcatcatgagcaccctcaccctggtggtgtggattgtcattggcttcattgactttggggtggtgcagagatacttccccaaccctaacaaacatatctcccaaacagaggtcattatcagatttgcctttcagacatctatcactgtgctgtgcattgcctgcccttgctctctgggcctggccacccccactgctgtgatggttggaacaggggtggctgcacaaaatgggatcctgatcaaagggggcaaacccctggaaatggcacataagattaaaacagttatgtttgataagacaggcaccattactcatggtgtccctagggtcatgagagtgctgctgctgggggatgtggctacactgccactgaggaaggtgctggctgtggtgggcacagctgaggcctcctctgaacacccactgggagttgctgtcacaaaatactgtaaggaggagctggggacagagaccctgggctattgtacagacttccaggctgtgcctggctgtggcattggctgtaaggtcagcaatgtggagggcatcctggcacactctgagagacccctctctgccccagcctcccacctgaatgaagctgggtctctgcctgctgagaaggatgctgtccctcagaccttctctgtgctgattgggaacagagagtggctgagaaggaatggcctgaccatcagctctgatgtctctgatgccatgacagatcatgagatgaaaggccagacagccattctggttgccattgatggtgtgctctgtggcatgattgccattgctgatgctgttaaacaggaagctgcactggcagtccacaccctccagtccatgggagtggatgtggtcctgattactggagacaacaggaaaacagctagggccattgctacccaagtggggatcaataaggtttttgcagaagtcctcccctcccacaaagtggccaaagtgcaggagctccagaacaagggtaaaaaggtggcaatggtgggagatggtgtgaatgacagcccagctctggcccaggctgacatgggagtggccattggtactggaacagatgtggccattgaagcagctgatgttgtcctcatcaggaatgatctcctggatgtggtggccagcatccatctgtccaagaggactgtgaggagaatcaggatcaacctggtcctggccctcatctataatctggtgggcatccccattgctgctggggtcttcatgcccattggcattgtgctgcagccctggatgggatctgctgccatggctgcttcttctgtctctgttgtgctgtccagcctgcagctgaagtgctataaaaagccagacctggagagatatgaagcccaggcccatggccatatgaagcccctcacagcttcccaggtctctgttcacattgggatggatgacagatggagggactccccaagggccacaccctgggaccaggtgtcctatgtgagccaggtgagcctcagctccctgacatctgataaaccctctaggcactctgctgctgcagatgatgatggtgacaaatggagcctcctcctgaatggcagagatgaggagcagtacatctaa

SEQ ID NO: 39 - DNA, 3495 bp, synthetic construct
  source 1..3495: mol_type = other DNA
atgcccgaacaggaaagacagattaccgcccgcgagggcgcttcccgcaaaattctgagcaaactgagcctgcccacccgcgcctgggaacctgccatgaagaaaagctttgcttttgataatgtcggctatgagggcggcctggacggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaa

SEQ ID NO: 40 - DNA, 3591 bp, synthetic construct
  source 1..3591: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggcccctgatggagccgaagggagtgggacagatcacaggtcttccagttctcattcccctggctccccaccgagaaaccaggtccagggcacatgcagtaccactctgattgccattgccggcatgacctgtgcatcctgtgtccattccattgaaggcatgatctcccaactggaaggggtgcagcaaatatcggtgtctttggccgaagggactgcaacagttctttataatccctctgtaattagcccagaagaactcagagctgctatagaagacatgggatttgaggcttcagtcgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 41 - DNA, 3108 bp, synthetic construct
  source 1..3108: mol_type = other DNA
atgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaa

SEQ ID NO: 42 - DNA, 3108 bp, synthetic construct
  source 1..3108: mol_type = other DNA
atgcctgaacaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcagccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcagggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagaaatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagccccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 43 - DNA, 3507 bp, synthetic construct
  source 1..3507: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcaggatcttgggcatgacttgccagtcatgtgtgaagtccattgaggacaggatttccaatttgaaaggcatcatcagcatgaaggtttccctggaacaaggcagtgccactgtgaaatatgtgccatcggttgtgtgcctgcaacaggtttgccatcaaattggggacatgggcttcgaggccagcattcctgatggagccgaagggagtgggacagatcacaggtcttccagttctcattcccctggctccccaccgagaaaccaggtccagggcacatgcagtaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtcttttcttgtgagtctgtttttctgtattcctgtaatggggctgatgatatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctccagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 44 - DNA, 3693 bp, synthetic construct
  source 1..3693: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggcccctgatggagccgaagggagtgggacagatcacaggtcttccagttctcattcccctggctccccaccgagaaaccaggtccagggcacatgcagtaccactctgattgccattgccggcatgacctgtgcatcctgtgtccattccattgaaggcatgatctcccaactggaaggggtgcagcaaatatcggtgtctttggccgaagggactgcaacagttctttataatccctctgtaattagcccagaagaactcagagctgctatagaagacatgggatttgaggcttcagtcgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtcttttcttgtgagtctgtttttctgtattcctgtaatggggctgatgatatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctccagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 45 - DNA, 3306 bp, synthetic construct
  source 1..3306: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggcccctgatggagccgaagggagtgggacagatcacaggtcttccagttctcattcccctggctccccaccgagaaaccaggtccagggcacatgcagtaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtcttttcttgtgagtctgtttttctgtattcctgtaatggggctgatgatatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctccagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 46 - DNA, 3501 bp, synthetic construct
  source 1..3501: mol_type = other DNA
atggcggcgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggccccagcagccaggtggcgaccaccttaatcgccatcgcaggcatgacctgcgcatcttgtgtgcatagcattgaaggcatgattagccagctggagggcgtgcagcagatctcagtctccctggctgaggggaccgccactgtcttgtacaatccgtctgttatttccccggaggagctgagagctgccattgaggacatgggatttgaagcatcggtggtgtctgagtcatgctccaccaatcccctgggaaatcactcagctggcaattcaatggttcagaccaccgatgggaccccaaccagcgtgcaggaagtggcaccccataccggcagactgcctgccaatcatgctcctgatatcctcgccaagagcccccagagcacccgcgcggtggcaccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaa

SEQ ID NO: 47 - DNA, 3501 bp, synthetic construct
  source 1..3501: mol_type = other DNA
atggcggcgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaa

SEQ ID NO: 48 - DNA, 3114 bp, synthetic construct
  source 1..3114: mol_type = other DNA
atggcggcgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaa

SEQ ID NO: 49 - DNA, 250 bp, synthetic construct
  source 1..250: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggca

SEQ ID NO: 50 - DNA, 250 bp, synthetic construct
  source 1..250: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttca

SEQ ID NO: 51 - DNA, 49 bp, synthetic construct
  source 1..49: mol_type = other DNA
aataaaagatctttattttcattagatctgtgtgttggttttttgtgtg

SEQ ID NO: 52 - DNA, 222 bp, synthetic construct
  source 1..222: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 53 - DNA, 101 bp, synthetic construct
  source 1..101: mol_type = other DNA
tccataaagtaggaaacactacatcatccataaagtaggaaacactacatcatccataaagtaggaaacactacatcatccataaagtaggaaacactaca

SEQ ID NO: 54 - DNA, 5100 bp, synthetic construct
  source 1..5100: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggccgcacgcgtcgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaagcggccgccaccatgcctgaacaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcaggatcttgggcatgacttgccagtcatgtgtgaagtccattgaggacaggatttccaatttgaaaggcatcatcagcatgaaggtttccctggaacaaggcagtgccactgtgaaatatgtgccatcggttgtgtgcctgcaacaggtttgccatcaaattggggacatgggcttcgaggccagcattgcagaaggaaaggcagcctcctggccctcaaggtccttgcctgcccaggaggctgtggtcaagctccgggtggagggcatgacctgccagtcctgtgtcagctccattgaaggcaaggtccggaaactgcaaggagtagtgagagtcaaagtctcactcagcaaccaagaggccgtcatcacttatcagccttatctcattcagcccgaagacctcagggaccatgtaaatgacatgggatttgaagctgccatcaagagcaaagtggctcccttaagcctgggaccaattgatattgagcggttacaaagcactaacccaaagagacctttatcttctgctaaccagaattttaataattctgagaccttggggcaccaaggaagccatgtggtcaccctccaactgagaatagatggaatgcattgtaagtcttgcgtcttgaatattgaagaaaatattggccagctcctaggggttcaaagtattcaagtctccttggagaacaaaactgcccaagtaaagtatgacccttcttgtaccagcccagtggctctgcagagggctatcgaggcacttccacctgggaattttaaagtttctcttcctgatggagccgaagggagtgggacagatcacaggtcttccagttctcattcccctggctccccaccgagaaaccaggtccagggcacatgcagtaccactctgattgccattgccggcatgacctgtgcatcctgtgtccattccattgaaggcatgatctcccaactggaaggggtgcagcaaatatcggtgtctttggccgaagggactgcaacagttctttataatcccgctgtaattagcccagaagaactcagagctgctatagaagacatgggatttgaggcttcagtcgtttctgaaagctgttctactaaccctcttggaaaccacagtgctgggaattccatggtgcaaactacagatggtacacctacatctctgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcagggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagaaatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagccccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgaaataaaagatctttattttcattagatctgtgtgttggttttttgtgtggatatcggatcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 55 - DNA, 3810 bp, synthetic construct
  source 1..3810: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggccgcacgcgtcgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaagcggccgccaccatgcctgaacaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcagccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcagggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagaaatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagccccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgaaataaaagatctttattttcattagatctgtgtgttggttttttgtgtggatatcggatcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 56 - DNA, 4130 bp, synthetic construct
  source 1..4130: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaacgcgtcgcctcgaggtcaattcacgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgcggccgccaccatgcctgaacaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcagccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcagggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagaaatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagccccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgaaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 57 - DNA, 4617 bp, synthetic construct
  source 1..4617: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaacgcgtcgcctcgaggtcaattcacgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgcgcggccgcaagatggcggcgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggcccctgatggagccgaagggagtgggacagatcacaggtcttccagttctcattcccctggctccccaccgagaaaccaggtccagggcacatgcagtaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtcttttcttgtgagtctgtttttctgtattcctgtaatggggctgatgatatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctccagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 58 - DNA, 4517 bp, synthetic construct
  source 1..4517: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaacgcgtcgcctcgaggtcaattcacgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgcggccgccaccatgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccactctgattgccattgccggcatgacctgtgcatcctgtgtccattccattgaaggcatgatctcccaactggaaggggtgcagcaaatatcggtgtctttggccgaagggactgcaacagttctttataatccctctgtaattagcccagaagaactcagagctgctatagaagacatgggatttgaggcttcagtcgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgaaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 59 - DNA, 4517 bp, synthetic construct
  source 1..4517: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaacgcgtcgcctcgaggtcaattcacgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccaggtggcgaccaccttaatcgccatcgcaggcatgacctgcgcatcttgtgtgcatagcattgaaggcatgattagccagctggagggcgtgcagcagatctcagtctccctggctgaggggaccgccactgtcttgtacaatccgtctgttatttccccggaggagctgagagctgccattgaggacatgggatttgaagcatcggtggtgtctgagtcatgctccaccaatcccctgggaaatcactcagctggcaattcaatggttcagaccaccgatgggaccccaaccagcgtgcaggaagtggcaccccataccggcagactgcctgccaatcatgctcctgatatcctcgccaagagcccccagagcacccgcgcggtggcaccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaaaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 60 - DNA, 4613 bp, synthetic construct
  source 1..4613: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaacgcgtcgcctcgaggtcaattcacgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgcggccgccaccatgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggcccctgatggagccgaagggagtgggacagatcacaggtcttccagttctcattcccctggctccccaccgagaaaccaggtccagggcacatgcagtaccactctgattgccattgccggcatgacctgtgcatcctgtgtccattccattgaaggcatgatctcccaactggaaggggtgcagcaaatatcggtgtctttggccgaagggactgcaacagttctttataatccctctgtaattagcccagaagaactcagagctgctatagaagacatgggatttgaggcttcagtcgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgaaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 61 - DNA, 4529 bp, synthetic construct
  source 1..4529: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaacgcgtcgcctcgaggtcaattcacgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgcggccgccaccatgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcaggatcttgggcatgacttgccagtcatgtgtgaagtccattgaggacaggatttccaatttgaaaggcatcatcagcatgaaggtttccctggaacaaggcagtgccactgtgaaatatgtgccatcggttgtgtgcctgcaacaggtttgccatcaaattggggacatgggcttcgaggccagcattcctgatggagccgaagggagtgggacagatcacaggtcttccagttctcattcccctggctccccaccgagaaaccaggtccagggcacatgcagtaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtcttttcttgtgagtctgtttttctgtattcctgtaatggggctgatgatatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctccagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgaaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 62 - DNA, 4715 bp, synthetic construct
  source 1..4715: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaacgcgtcgcctcgaggtcaattcacgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgcggccgccaccatgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggcccctgatggagccgaagggagtgggacagatcacaggtcttccagttctcattcccctggctccccaccgagaaaccaggtccagggcacatgcagtaccactctgattgccattgccggcatgacctgtgcatcctgtgtccattccattgaaggcatgatctcccaactggaaggggtgcagcaaatatcggtgtctttggccgaagggactgcaacagttctttataatccctctgtaattagcccagaagaactcagagctgctatagaagacatgggatttgaggcttcagtcgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtcttttcttgtgagtctgtttttctgtattcctgtaatggggctgatgatatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctccagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgaaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 63 - DNA, 4328 bp, synthetic construct
  source 1..4328: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaacgcgtcgcctcgaggtcaattcacgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgcggccgccaccatgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggcccctgatggagccgaagggagtgggacagatcacaggtcttccagttctcattcccctggctccccaccgagaaaccaggtccagggcacatgcagtaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtcttttcttgtgagtctgtttttctgtattcctgtaatggggctgatgatatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctccagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgaaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 64 - DNA, 4128 bp, synthetic construct
  source 1..4128: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaaaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 65 - DNA, 4410 bp, synthetic construct
  source 1..4410: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 66 - DNA, 4162 bp, synthetic construct
  source 1..4162: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagaattttaataaatacctctctgtccttctcctagcttcaccctttgtttgttgaaaggagatactttagtgcagtattgcaacagcttatgtagacatgcaaccttgaggtcacagacctatgagggattgagacaaaggcaaatttagcaaatattttccagagtactctgctgcaataacgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaaaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 67 - DNA, 4106 bp, synthetic construct
  source 1..4106: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaaaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 68 - DNA, 4586 bp, synthetic construct
  source 1..4586: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccaccatgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggcccctgatggagccgaagggagtgggacagatcacaggtcttccagttctcattcccctggctccccaccgagaaaccaggtccagggcacatgcagtaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtcttttcttgtgagtctgtttttctgtattcctgtaatggggctgatgatatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctccagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 69 - DNA, 4304 bp, synthetic construct
  source 1..4304: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccaccatgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggcccctgatggagccgaagggagtgggacagatcacaggtcttccagttctcattcccctggctccccaccgagaaaccaggtccagggcacatgcagtaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtcttttcttgtgagtctgtttttctgtattcctgtaatggggctgatgatatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctccagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgaaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 70 - DNA, 4775 bp, synthetic construct
  source 1..4775: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccaccatgcctgaacaagaaagacagatcacagcaagagaaggagcatctaggaagatcctgagcaagctctccctgcccacaagggcctgggagcctgccatgaagaagtcttttgcctttgataatgtgggttatgaaggagggctggatggcctgggccccagcagccaggtggcaaccaccctgattgccattgcaggcatgacctgtgcatcttgtgtgcatagcattgaaggcatgattagccagctggagggagtgcagcagatctctgtctccctggctgaggggacagccactgtcctgtacaatccctctgttatttcccctgaggagctgagagctgccattgaggacatgggatttgaagcatctgtggtgtctgagtcctgctccaccaatcccctgggaaatcactctgctggcaattctatggttcagaccacagatgggaccccaacctctgtgcaggaagtggcaccccatactggcagactgcctgccaatcatgctcctgatatcctggccaagagcccccagagcaccagggctgtggcaccccagaagtgtttcctgcagatcaagggcatgacctgtgccagctgtgttagcaatattgaaagaaacctccaaaaagaggctggagttctgtctgttctggtggctctgatggctgggaaagcagagatcaagtatgatccagaggtgattcagccactggagattgcccagttcatccaggatctgggctttgaggcagctgtgatggaggattatgctggctctgatggcaacattgaactgaccatcacaggcatgacctgtgcttcctgtgtgcataatattgagagcaaactgaccagaaccaatggaatcacctatgcctctgttgccctggcaacctctaaggctctggtgaagtttgaccctgaaatcattggacctagagacatcatcaaaattattgaagagattggtttccatgccagcctggctcagagaaatcccaatgcacaccatctggaccataagatggaaatcaaacagtggaagaagagcttcctgtgtagcctggtctttgggattcctgtcatggcactgatgatttacatgctcattccctccaatgagccccaccagtccatggtgctggaccacaacatcatccctgggctgtctatcctcaacctgatcttctttattctgtgcacatttgtccagctcctgggaggctggtatttctatgttcaggcctacaaatccctgaggcatagatctgccaacatggatgttctgattgtgctggctacatccattgcctatgtgtactccctggtgatcctggtggttgctgtggctgaaaaggctgagagaagccctgtcaccttctttgacaccccccccatgctctttgtgtttattgcactgggtaggtggctggagcatctggccaagtccaagacatctgaggctctggccaagctgatgtctctccaggccactgaggccactgtggtgactctgggtgaagacaatctgatcattagagaggaacaggtgcctatggagctggtgcagagaggagatattgtcaaggtggtgccaggaggcaagttcccagtggatggaaaagtgctggagggcaataccatggctgatgaatccctcatcactggggaggcaatgcctgtgaccaagaaacctggaagcactgtcattgcaggcagcatcaatgcccatgggtctgtcctgatcaaggccacccatgtgggcaatgatacaactctggcccagattgtcaagctggtggaagaggctcagatgtctaaagctccaatccagcaactggctgacagattctctggctactttgtcccctttatcatcatcatgagcaccctcaccctggtggtgtggattgtcattggcttcattgactttggggtggtgcagagatacttccccaaccctaacaaacatatctcccaaacagaggtcattatcagatttgcctttcagacatctatcactgtgctgtgcattgcctgcccttgctctctgggcctggccacccccactgctgtgatggttggaacaggggtggctgcacaaaatgggatcctgatcaaagggggcaaacccctggaaatggcacataagattaaaacagttatgtttgataagacaggcaccattactcatggtgtccctagggtcatgagagtgctgctgctgggggatgtggctacactgccactgaggaaggtgctggctgtggtgggcacagctgaggcctcctctgaacacccactgggagttgctgtcacaaaatactgtaaggaggagctggggacagagaccctgggctattgtacagacttccaggctgtgcctggctgtggcattggctgtaaggtcagcaatgtggagggcatcctggcacactctgagagacccctctctgccccagcctcccacctgaatgaagctgggtctctgcctgctgagaaggatgctgtccctcagaccttctctgtgctgattgggaacagagagtggctgagaaggaatggcctgaccatcagctctgatgtctctgatgccatgacagatcatgagatgaaaggccagacagccattctggttgccattgatggtgtgctctgtggcatgattgccattgctgatgctgttaaacaggaagctgcactggcagtccacaccctccagtccatgggagtggatgtggtcctgattactggagacaacaggaaaacagctagggccattgctacccaagtggggatcaataaggtttttgcagaagtcctcccctcccacaaagtggccaaagtgcaggagctccagaacaagggtaaaaaggtggcaatggtgggagatggtgtgaatgacagcccagctctggcccaggctgacatgggagtggccattggtactggaacagatgtggccattgaagcagctgatgttgtcctcatcaggaatgatctcctggatgtggtggccagcatccatctgtccaagaggactgtgaggagaatcaggatcaacctggtcctggccctcatctataatctggtgggcatccccattgctgctggggtcttcatgcccattggcattgtgctgcagccctggatgggatctgctgccatggctgcttcttctgtctctgttgtgctgtccagcctgcagctgaagtgctataaaaagccagacctggagagatatgaagcccaggcccatggccatatgaagcccctcacagcttcccaggtctctgttcacattgggatggatgacagatggagggactccccaagggccacaccctgggaccaggtgtcctatgtgagccaggtgagcctcagctccctgacatctgataaaccctctaggcactctgctgctgcagatgatgatggtgacaaatggagcctcctcctgaatggcagagatgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 71 - DNA, 4433 bp, synthetic construct
  source 1..4433: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaactcctaaaaaaccgccaccatggcggcgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccaggtggccaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 72 - DNA, 4433 bp, synthetic construct
  source 1..4433: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaactcctaaaaaaccgccaagatggcggccccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccaggtggccaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 73 - DNA, 4894 bp, synthetic construct
  source 1..4894: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaactcctaaaaaaccgccaccatgcccgaacaggaaagacagattaccgcccgcgagggcgcttcccgcaaaattctgagcaaactgagcctgcccacccgcgcctgggaacctgccatgaagaaaagctttgcttttgataatgtcggctatgagggcggcctggacggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaagtccataaagtaggaaacactacatcatccataaagtaggaaacactacatcatccataaagtaggaaacactacatcatccataaagtaggaaacactacagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 74 - DNA, 4894 bp, synthetic construct
  source 1..4894: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaactcctaaaaaaccgccaccatgcccgaacaggaaagacagattaccgcccgcgagggcgcttcccgcaaaattctgagcaaactgagcctgcccacccgcgcctgggaacctgccatgaagaaaagctttgcttttgataatgtcggctatgagggcggcctggacggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaagtccataaagtaggaaacactacatcatccataaagtaggaaacactacatcatccataaagtaggaaacactacatcatccataaagtaggaaacactacagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 75 - DNA, 4644 bp, synthetic construct
  source 1..4644: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaactcctaaaaaaccgccaccatgcccgaacaggaaagacagattaccgcccgcgagggcgcttcccgcaaaattctgagcaaactgagcctgcccacccgcgcctgggaacctgccatgaagaaaagctttgcttttgataatgtcggctatgagggcggcctggacggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaagtccataaagtaggaaacactacatcatccataaagtaggaaacactacatcatccataaagtaggaaacactacatcatccataaagtaggaaacactacagaattcgatatcaagcttatcgatcctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 76 - DNA, 4793 bp, synthetic construct
  source 1..4793: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaactcctaaaaaaccgccaccatgcccgaacaggaaagacagattaccgcccgcgagggcgcttcccgcaaaattctgagcaaactgagcctgcccacccgcgcctgggaacctgccatgaagaaaagctttgcttttgataatgtcggctatgagggcggcctggacggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 77 - DNA, 4545 bp, synthetic construct
  source 1..4545: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaactcctaaaaaaccgccaccatgcccgaacaggaaagacagattaccgcccgcgagggcgcttcccgcaaaattctgagcaaactgagcctgcccacccgcgcctgggaacctgccatgaagaaaagctttgcttttgataatgtcggctatgagggcggcctggacggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaaggaattcgatatcaagcttatcgatcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 78 - DNA, 4388 bp, synthetic construct
  source 1..4388: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 79 - DNA, 4592 bp, synthetic construct
  source 1..4592: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccaccatggcggcgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggcccctgatggagccgaagggagtgggacagatcacaggtcttccagttctcattcccctggctccccaccgagaaaccaggtccagggcacatgcagtaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtcttttcttgtgagtctgtttttctgtattcctgtaatggggctgatgatatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctccagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 80 - DNA, 4775 bp, synthetic construct
  source 1..4775: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccaggtggcgaccaccttaatcgccatcgcaggcatgacctgcgcatcttgtgtgcatagcattgaaggcatgattagccagctggagggcgtgcagcagatctcagtctccctggctgaggggaccgccactgtcttgtacaatccgtctgttatttccccggaggagctgagagctgccattgaggacatgggatttgaagcatcggtggtgtctgagtcatgctccaccaatcccctgggaaatcactcagctggcaattcaatggttcagaccaccgatgggaccccaaccagcgtgcaggaagtggcaccccataccggcagactgcctgccaatcatgctcctgatatcctcgccaagagcccccagagcacccgcgcggtggcaccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 81 - DNA, 4410 bp, synthetic construct
  source 1..4410: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 82 - DNA, 4415 bp, synthetic construct
  source 1..4415: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaagatggcggcgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 83 - DNA, 4802 bp, synthetic construct
  source 1..4802: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaagatggcggcgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggccccagcagccaggtggcgaccaccttaatcgccatcgcaggcatgacctgcgcatcttgtgtgcatagcattgaaggcatgattagccagctggagggcgtgcagcagatctcagtctccctggctgaggggaccgccactgtcttgtacaatccgtctgttatttccccggaggagctgagagctgccattgaggacatgggatttgaagcatcggtggtgtctgagtcatgctccaccaatcccctgggaaatcactcagctggcaattcaatggttcagaccaccgatgggaccccaaccagcgtgcaggaagtggcaccccataccggcagactgcctgccaatcatgctcctgatatcctcgccaagagcccccagagcacccgcgcggtggcaccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 84 - DNA, 4613 bp, synthetic construct
  source 1..4613: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaagatggcggcgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggcccctgatggagccgaagggagtgggacagatcacaggtcttccagttctcattcccctggctccccaccgagaaaccaggtccagggcacatgcagtaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtcttttcttgtgagtctgtttttctgtattcctgtaatggggctgatgatatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctccagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 85 - DNA, 4904 bp, synthetic construct
  source 1..4904: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaagatggcggcgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaagtccataaagtaggaaacactacatcatccataaagtaggaaacactacatcatccataaagtaggaaacactacatcatccataaagtaggaaacactacagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 86 - DNA, 4393 bp, synthetic construct
  source 1..4393: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaagatggcggcgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 87 - DNA, 4780 bp, synthetic construct
  source 1..4780: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaagatggcggcgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggccccagcagccaggtggcgaccaccttaatcgccatcgcaggcatgacctgcgcatcttgtgtgcatagcattgaaggcatgattagccagctggagggcgtgcagcagatctcagtctccctggctgaggggaccgccactgtcttgtacaatccgtctgttatttccccggaggagctgagagctgccattgaggacatgggatttgaagcatcggtggtgtctgagtcatgctccaccaatcccctgggaaatcactcagctggcaattcaatggttcagaccaccgatgggaccccaaccagcgtgcaggaagtggcaccccataccggcagactgcctgccaatcatgctcctgatatcctcgccaagagcccccagagcacccgcgcggtggcaccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 88 - DNA, 4591 bp, synthetic construct
  source 1..4591: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaagatggcggcgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggcccctgatggagccgaagggagtgggacagatcacaggtcttccagttctcattcccctggctccccaccgagaaaccaggtccagggcacatgcagtaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtcttttcttgtgagtctgtttttctgtattcctgtaatggggctgatgatatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctccagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 89 - DNA, 4882 bp, synthetic construct
  source 1..4882: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaagatggcggcgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaagtccataaagtaggaaacactacatcatccataaagtaggaaacactacatcatccataaagtaggaaacactacatcatccataaagtaggaaacactacagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 90 - DNA, 4591 bp, synthetic construct
  source 1..4591: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttgctggtgcttccccaggctggagattgagttaatattaacaggcccaaggcgatgtgggcttgtgcaatcataggcccggcctcgccaggtcacctgaggagttaatgaatacatatctcctgccgcgttcgccccgcccccgacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgcaagatggcggcgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggcccctgatggagccgaagggagtgggacagatcacaggtcttccagttctcattcccctggctccccaccgagaaaccaggtccagggcacatgcagtaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtcttttcttgtgagtctgtttttctgtattcctgtaatggggctgatgatatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctccagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 91 - DNA, 4442 bp, synthetic construct
  source 1..4442: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagaattttaataaatacctctctgtccttctcctagcttcaccctttgtttgttgaaaggagatactttagtgcagtattgcaacagcttatgtagacatgcaaccttgaggtcacagacctatgagggattgagacaaaggcaaatttagcaaatattttccagagtactctgctgcaataaccgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 92 - DNA, 4724 bp, synthetic construct
  source 1..4724: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagacaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaagcggccgccaccgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 93 - DNA, 4583 bp, synthetic construct
  source 1..4583: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagagagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcgagccgagtggtcgtgcctccatagaagcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 94 - DNA, 4360 bp, synthetic construct
  source 1..4360: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagatcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccggatccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 95 - DNA, 4849 bp, synthetic construct
  source 1..4849: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagatcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccggatccgccaccatgcccgaacaggaaagacagattaccgcccgcgagggcgcttcccgcaaaattctgagcaaactgagcctgcccacccgcgcctgggaacctgccatgaagaaaagctttgcttttgataatgtcggctatgagggcggcctggacggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaagtccataaagtaggaaacactacatcatccataaagtaggaaacactacatcatccataaagtaggaaacactacatcatccataaagtaggaaacactacagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 96 - DNA, 4564 bp, synthetic construct
  source 1..4564: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagatcccataaaactctagcctcccagagggaccccagcccccaccctcccgcccacgaacccctgcatttccagaatcagccccagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctagccctccccgagcgccctgcctccgagggccggcgcactataaaagccccttcaccaggagaagccgtcacacagatccacaagctcgagccgagtggtcgtgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccggatccgccaccatggcggcgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggcccctgatggagccgaagggagtgggacagatcacaggtcttccagttctcattcccctggctccccaccgagaaaccaggtccagggcacatgcagtaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtcttttcttgtgagtctgtttttctgtattcctgtaatggggctgatgatatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctccagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 97 - DNA, 4449 bp, synthetic construct
  source 1..4449: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagatcccataaaactctagcctcccagagggacccgagttgctggtgcttccccaggctggagattgagttaatattaacaggcccaaggcgatgtgggcttgtgcaatcataggcccggcctcgccaggtcacctgaggagttaatgaatacatatctcctgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggttaatcattaagtcgttaatttttgtggcccttgcgatgtttgctctggttaataatctcaggacaaacagaggttaataattttccagatctctctgagcaatagtataaaaggccagcagcagcctgaccacatctcatccttgagccaagaggtaagggtttaagggatggttggttggtggggtattaatgtttaattacctggagcacctgcctgaaatcactttttttcaggttggggatccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 98 - DNA, 4451 bp, synthetic construct
  source 1..4451: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaggtaccgagttgctggtgcttccccaggctggagattgagttaatattaacaggcccaaggcgatgtgggcttgtgcaatcataggcccggcctcgccaggtcacctgaggagttaatgaatacatatctcctgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggttaatcattaagtcgttaatttttgtggcccttgcgatgtttgctctggttaataatctcaggacaaacagaggttaataattttccagatctctctgagcaatagtataaaaggccagcagcagcctgaccacatctcatcctgatccacaagctcgagccgagtggtcgttgagccaagaggtaagggtttaagggatggttggttggtggggtattaatgtttaattacctggagcacctgcctgaaatcactttttttcaggttggggatccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 99 - DNA, 4940 bp, synthetic construct
  source 1..4940: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaggtaccgagttgctggtgcttccccaggctggagattgagttaatattaacaggcccaaggcgatgtgggcttgtgcaatcataggcccggcctcgccaggtcacctgaggagttaatgaatacatatctcctgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggttaatcattaagtcgttaatttttgtggcccttgcgatgtttgctctggttaataatctcaggacaaacagaggttaataattttccagatctctctgagcaatagtataaaaggccagcagcagcctgaccacatctcatcctgatccacaagctcgagccgagtggtcgttgagccaagaggtaagggtttaagggatggttggttggtggggtattaatgtttaattacctggagcacctgcctgaaatcactttttttcaggttggggatccgccaccatgcccgaacaggaaagacagattaccgcccgcgagggcgcttcccgcaaaattctgagcaaactgagcctgcccacccgcgcctgggaacctgccatgaagaaaagctttgcttttgataatgtcggctatgagggcggcctggacggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaagtccataaagtaggaaacactacatcatccataaagtaggaaacactacatcatccataaagtaggaaacactacatcatccataaagtaggaaacactacagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 100 - DNA, 4113 bp, synthetic construct
  source 1..4113: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaggtaccgttaatcattaagtcgttaatttttgtggcccttgcgatgtttgctctggttaataatctcaggacaaacagaggttaataattttccagatctctctgagcaatagtataaaaggccagcagcagcctgaccacatctcatccttgatccacaagctcgagccgagtggtcgtgagccaagaggtaagggtttaagggatggttggttggtggggtattaatgtttaattacctggagcacctgcctgaaatcactttttttcaggttggggatccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 101 - DNA, 4602 bp, synthetic construct
  source 1..4602: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaggtaccgttaatcattaagtcgttaatttttgtggcccttgcgatgtttgctctggttaataatctcaggacaaacagaggttaataattttccagatctctctgagcaatagtataaaaggccagcagcagcctgaccacatctcatccttgatccacaagctcgagccgagtggtcgtgagccaagaggtaagggtttaagggatggttggttggtggggtattaatgtttaattacctggagcacctgcctgaaatcactttttttcaggttggggatccgccaccatgcccgaacaggaaagacagattaccgcccgcgagggcgcttcccgcaaaattctgagcaaactgagcctgcccacccgcgcctgggaacctgccatgaagaaaagctttgcttttgataatgtcggctatgagggcggcctggacggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaagtccataaagtaggaaacactacatcatccataaagtaggaaacactacatcatccataaagtaggaaacactacatcatccataaagtaggaaacactacagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 102 - DNA, 4319 bp, synthetic construct
  source 1..4319: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaggtaccgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgggtacccgggggcgcgccagtcaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcggcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 103 - DNA, 4808 bp, synthetic construct
  source 1..4808: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaggtaccgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgggtacccgggggcgcgccagtcaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcggcggccgccaccatgcccgaacaggaaagacagattaccgcccgcgagggcgcttcccgcaaaattctgagcaaactgagcctgcccacccgcgcctgggaacctgccatgaagaaaagctttgcttttgataatgtcggctatgagggcggcctggacggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaagtccataaagtaggaaacactacatcatccataaagtaggaaacactacatcatccataaagtaggaaacactacatcatccataaagtaggaaacactacagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 104 - DNA, 4411 bp, synthetic construct
  source 1..4411: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaggtaccgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggaccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaagaggtaagggtttaagggatggttggttggtggggtattaatgtttaattacctggagcacctgcctgaaatcactttttttcaggttgggcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 105 - DNA, 4900 bp, synthetic construct
  source 1..4900: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaggtaccgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggaccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaagaggtaagggtttaagggatggttggttggtggggtattaatgtttaattacctggagcacctgcctgaaatcactttttttcaggttgggcggccgccaccatgcccgaacaggaaagacagattaccgcccgcgagggcgcttcccgcaaaattctgagcaaactgagcctgcccacccgcgcctgggaacctgccatgaagaaaagctttgcttttgataatgtcggctatgagggcggcctggacggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaagtccataaagtaggaaacactacatcatccataaagtaggaaacactacatcatccataaagtaggaaacactacatcatccataaagtaggaaacactacagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 106 - DNA, 4774 bp, synthetic construct
  source 1..4774: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcggcggccgccaccatgcccgaacaggaaagacagattaccgcccgcgagggcgcttcccgcaaaattctgagcaaactgagcctgcccacccgcgcctgggaacctgccatgaagaaaagctttgcttttgataatgtcggctatgagggcggcctggacggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaagtccataaagtaggaaacactacatcatccataaagtaggaaacactacatcatccataaagtaggaaacactacatcatccataaagtaggaaacactacagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 107 - DNA, 4774 bp, synthetic construct; identical to SEQ ID NO: 106
  source 1..4774: mol_type = other DNA

SEQ ID NO: 108 - DNA, 4378 bp, synthetic construct
  source 1..4378: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaggtaccgagttaataattaccagcgcgggccaaataaataatccgcgaggggcagctggactttggactccgcgcgctggtaattattaacctccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcggcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 109 - DNA, 4867 bp, synthetic construct
  source 1..4867: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaggtaccgagttaataattaccagcgcgggccaaataaataatccgcgaggggcagctggactttggactccgcgcgctggtaattattaacctccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcggcggccgccaccatgcccgaacaggaaagacagattaccgcccgcgagggcgcttcccgcaaaattctgagcaaactgagcctgcccacccgcgcctgggaacctgccatgaagaaaagctttgcttttgataatgtcggctatgagggcggcctggacggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaagtccataaagtaggaaacactacatcatccataaagtaggaaacactacatcatccataaagtaggaaacactacatcatccataaagtaggaaacactacagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 110 - DNA, 4463 bp, synthetic construct
  source 1..4463: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaggtaccgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgggtacccgggggcgcgccagtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcggcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 111 - DNA, 4952 bp, synthetic construct
  source 1..4952: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaggtaccgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgggtacccgggggcgcgccagtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcggcggccgccaccatgcccgaacaggaaagacagattaccgcccgcgagggcgcttcccgcaaaattctgagcaaactgagcctgcccacccgcgcctgggaacctgccatgaagaaaagctttgcttttgataatgtcggctatgagggcggcctggacggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaagtccataaagtaggaaacactacatcatccataaagtaggaaacactacatcatccataaagtaggaaacactacatcatccataaagtaggaaacactacagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 112 - DNA, 4424 bp, synthetic construct
  source 1..4424: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaggtaccgagttaataattaccagcgcgggccaaataaataatccgcgccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaagaggtaagggtttaagggatggttggttggtggggtattaatgtttaattacctggagcacctgcctgaaatcactttttttcaggttgggcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 113 - DNA, 4913 bp, synthetic construct
  source 1..4913: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaggtaccgagttaataattaccagcgcgggccaaataaataatccgcgccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaagaggtaagggtttaagggatggttggttggtggggtattaatgtttaattacctggagcacctgcctgaaatcactttttttcaggttgggcggccgccaccatgcccgaacaggaaagacagattaccgcccgcgagggcgcttcccgcaaaattctgagcaaactgagcctgcccacccgcgcctgggaacctgccatgaagaaaagctttgcttttgataatgtcggctatgagggcggcctggacggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaagtccataaagtaggaaacactacatcatccataaagtaggaaacactacatcatccataaagtaggaaacactacatcatccataaagtaggaaacactacagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 114 - DNA, 4555 bp, synthetic construct
  source 1..4555: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaggtaccgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaagaggtaagggtttaagggatggttggttggtggggtattaatgtttaattacctggagcacctgcctgaaatcactttttttcaggttgggcggccgccaccatgccggaacaagaacgccagatcacagcaagagaaggagcaagtaggaagatcctgagcaagctctccctgcccacaagggcctgggagccggccatgaagaagtcttttgccttcgataatgtcggttatgaaggcgggctcgatggcctgggccccagcagccagccccagaagtgtttcctgcagatcaagggcatgacctgtgccagctgtgttagcaatatcgaaagaaacctccaaaaagaggctggagttctgagtgttctagtggctttaatggccgggaaagccgagatcaagtatgatcccgaggtgattcagccattggagattgcccagttcatccaggatctgggcttcgaggccgccgtgatggaggattacgccggctctgatggcaacatcgaactgaccatcaccggcatgacctgtgcttcctgcgtgcataatatcgagagcaaactgacgcggaccaatggaatcacctatgccagcgtcgccctggcaacctctaaggctctggtgaagtttgaccccgaaatcattggacctagagacatcatcaaaattattgaagagattggtttccacgccagcttagctcagcggaatcccaacgcacaccatcttgaccataagatggaaatcaaacagtggaagaagagcttcttgtgtagcctggtcttcgggatacccgtcatggcactgatgatttacatgctcattccgtccaacgagccccaccagtccatggtgctggaccacaacatcatccctgggctgtctatcctcaacctgatcttctttattctgtgcacatttgtccagctcttgggaggctggtatttctatgtacaggcctacaaatccctgaggcatcgaagcgccaacatggacgttttaattgtgctggctacatccattgcctacgtgtactccctggtgatcttagtggttgctgtggctgaaaaggctgagagaagccctgtcaccttcttcgacaccccccccatgctcttcgtgtttattgcactgggtcgctggctggagcatttagccaagtccaagacatccgaggctctggccaagctgatgtctctacaggccacggaggccaccgtggtgactctgggtgaagacaatttgataatacgcgaggaacaggtgcctatggagcttgtgcagcgcggagatatcgtcaaggtggtgcccggcggcaagttcccagtggatggaaaagtgttagagggcaataccatggccgatgaatccctcatcactggcgaggcaatgccggtgaccaagaaacctggaagtactgtcattgcaggcagcatcaatgcccatgggagcgtcctgatcaaggccacccacgtgggcaatgatacaactctcgcccagatcgtcaagctagtggaagaggctcagatgtctaaagctccaatccagcaactggctgaccgcttcagcggctactttgtcccctttatcatcatcatgagcaccctcaccctggtggtgtggatagtcataggcttcattgactttggcgtggtgcagagatacttccccaaccctaacaaacatatctcccaaaccgaggtcattatcagattcgcctttcagacatctatcactgtgctgtgcatagcctgcccttgctctctgggcctggccacccccaccgccgtgatggttggaaccggggtcgctgcacaaaatgggatcctgataaaagggggcaaacccctggaaatggcacataagattaaaacagttatgtttgataagacaggcaccattactcacggtgtccctcgagtcatgagagtgctgctgctgggggacgtggctacactgccactgcgcaaggtgctggctgtggtgggcacagcggaggcctcctcagaacacccattaggcgtagctgtcacaaaatactgtaaggaggagctggggacagagaccctcggctattgtacagacttccaggccgtgcctggctgcggcatcggctgtaaggtcagcaatgtggagggcatcctggcacacagcgagagacccctctctgccccagcctcccacctgaatgaagctgggtctctgcctgctgagaaggacgccgtccctcagaccttctccgtgttgattgggaacagagagtggcttagaaggaatggcctgaccatcagctctgacgtctcggatgccatgacagatcacgagatgaaaggccagacagccatactggttgccatcgacggcgtgctctgtggcatgattgccatcgctgatgctgttaaacaggaagctgcgctggcagtccacaccctccagtccatgggagtggacgtggtcctgattactggagacaacaggaaaaccgctcgggccatcgctacccaagtggggataaataaggtttttgcagaagtcttaccctcccacaaagtagccaaagtgcaggagctccagaacaagggtaaaaaggtggcaatggtgggagatggtgtgaatgacagcccagctctcgcgcaggctgacatgggcgtagccataggtactggaacagatgtcgcgattgaagccgctgacgtagtcctcatccgaaacgatttgctggatgtggtggccagcatccatctgtccaagcgtaccgtgcgccggatccgcatcaacctggtcttggccctcatctataatctcgtgggcatccccatcgctgctggggtcttcatgcccatcggcattgtgctgcagccctggatgggaagcgctgccatggctgcttcttctgtctccgttgtgctgtccagcctacagctgaagtgctataaaaagccagacctcgagcgctacgaagcccaggcgcatggccatatgaagcccctcacggcttcccaggtctcggttcacattgggatggatgacagatggcgggactccccacgcgccacaccctgggaccaggtatcctatgtgagccaggtgagcctcagctccctgacatcagataaaccgtctaggcactcagctgccgcagatgatgacggtgacaaatggagcctcctcctgaacggccgcgacgaggagcagtacatctaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 115 - DNA, 5044 bp, synthetic construct
  source 1..5044: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagaggtaccgagttaataattaccagcgcgggccaaataaataatccgcgaggggcagctggactttggactccgcgcgctggtaattattaacctcgcgaatattgattcgaggccgcgattgccgcaatcgcgaggggcaggtgacctttgcccagcgcgcgttcgccccgccccggacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaagaggtaagggtttaagggatggttggttggtggggtattaatgtttaattacctggagcacctgcctgaaatcactttttttcaggttgggcggccgccaccatgcccgaacaggaaagacagattaccgcccgcgagggcgcttcccgcaaaattctgagcaaactgagcctgcccacccgcgcctgggaacctgccatgaagaaaagctttgcttttgataatgtcggctatgagggcggcctggacggcctgggccctagcagccaggtggctaccaccctgattgccattgccggcatgacctgcgccagctgcgtgcatagcattgagggcatgattagccagctggagggcgtgcagcagatttccgtgagcctggctgagggcaccgctaccgtgctgtataatccctccgtgattagccccgaagaactgcgcgctgccattgaagatatgggctttgaagcctccgtggtgtccgaaagctgcagcaccaatcccctgggcaatcattccgccggcaatagcatggtgcagaccaccgacggcacccccacctccgtgcaggaagtggctccccataccggccgcctgcccgccaatcatgcccctgatattctggctaaaagcccccagagcacccgcgccgtggctcctcagaaatgctttctgcagattaagggcatgacctgcgctagctgcgtgagcaatattgaacgcaatctgcagaaagaagccggcgtgctgtccgtgctggtggccctgatggccggcaaagctgaaatcaaatatgatcccgaagtgattcagcccctggaaattgcccagttcattcaggatctgggctttgaggctgccgtgatggaagattatgccggctccgacggcaatattgaactgaccattaccggcatgacctgcgccagctgcgtgcataatattgaaagcaaactgacccgcaccaacggcattacctatgcttccgtggccctggctaccagcaaagccctggtgaaatttgatcctgaaattatcggcccccgcgatattatcaaaattattgaagaaatcggctttcacgccagcctggctcagcgcaatcccaatgcccatcatctggatcataagatggaaatcaaacagtggaagaaaagctttctgtgcagcctggtgttcggcattcctgtgatggccctgatgatctatatgctgattcccagcaatgaaccccatcagagcatggtgctggatcataatattattcccggcctgagcattctgaatctgattttctttatcctgtgcacctttgtgcagctgctgggcggctggtatttctatgtgcaggcttataaaagcctgcgccaccgctccgccaatatggatgtgctgattgtgctggccaccagcattgcttatgtctatagcctggtcattctggtggtggccgtggccgagaaagctgaacgcagccccgtgaccttctttgatacccctcccatgctgttcgtgtttatcgctctgggccgctggctggaacatctggccaaaagcaaaacctccgaagccctggctaaactgatgagcctgcaggccaccgaagccaccgtggtgaccctgggcgaagataatctgattatccgcgaagaacaggtgcctatggaactggtgcagcgcggcgatattgtgaaagtggtgcccggcggcaaatttcccgtggacggcaaagtgctggagggcaataccatggctgatgaatccctgattaccggcgaagctatgcctgtgaccaagaaacccggcagcaccgtgattgccggcagcattaatgctcacggctccgtgctgatcaaggctacccatgtcggcaatgataccaccctggcccagattgtgaaactggtggaagaagcccagatgagcaaagcccctattcagcagctggctgaccgcttttccggctattttgtgccctttatcattattatgagcaccctgaccctggtggtgtggattgtgatcggcttcattgatttcggcgtggtgcagagatattttcctaatcccaacaaacatattagccagaccgaagtgattatccgctttgcttttcagaccagcattaccgtgctgtgcattgcttgcccctgcagcctgggcctggctacccccaccgccgtcatggtcggcaccggcgtggctgcccagaacggcattctgattaagggcggcaaacctctggaaatggcccacaaaatcaaaaccgtgatgtttgataaaaccggcaccattacccacggcgtgccccgcgtgatgcgcgtgctgctgctgggcgatgtggctaccctgcccctgcgcaaagtgctggccgtggtcggcaccgccgaagctagctccgaacatcccctgggcgtggccgtgaccaaatattgcaaagaagaactgggcaccgaaaccctgggctattgcaccgattttcaggccgtccctggctgcggcatcggctgcaaagtgagcaatgtggagggcattctggcccattccgaaagacccctgtccgcccctgctagccatctgaatgaagccggcagcctgcctgctgagaaagatgccgtgccccagaccttttccgtgctgatcggcaaccgcgaatggctgcgccgcaacggcctgaccattagctccgatgtctccgatgccatgaccgatcatgaaatgaaaggccagaccgccattctggtggccattgacggcgtgctgtgcggcatgattgccattgccgatgccgtgaaacaggaggctgccctggctgtgcataccctgcagagcatgggcgtggatgtggtgctgattaccggcgataaccgcaaaaccgctcgcgccattgccacccaggtcggcattaacaaagtgtttgctgaagtgctgcccagccataaagtggctaaagtgcaggaactgcagaacaaaggcaagaaagtggctatggtcggcgacggcgtgaatgatagccctgccctggcccaggccgatatgggcgtggctatcggcaccggcaccgatgtggctattgaggctgccgatgtggtgctgatccgcaatgatctgctggatgtggtggctagcattcatctgagcaaacgcaccgtccgccgcatccgcattaatctggtgctggccctgatctataatctggtcggcattcccatcgccgccggcgtgtttatgcccatcggcattgtgctgcagccctggatgggcagcgccgctatggccgctagctccgtgtccgtggtgctgagcagcctgcagctgaaatgctataagaaacccgatctggaaagatatgaagctcaggctcacggccatatgaaacccctgaccgccagccaggtgtccgtgcatatcggcatggatgaccgctggcgcgatagcccccgcgccaccccttgggatcaggtgagctatgtgagccaggtgtccctgagctccctgacctccgataaaccctcccgccattccgccgctgccgatgatgacggcgataagtggagcctgctgctgaacggccgcgatgaagaacagtatatctaagtccataaagtaggaaacactacatcatccataaagtaggaaacactacatcatccataaagtaggaaacactacatcatccataaagtaggaaacactacagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcaggaataaaagatctttattttcattagatctgtgtgttggttttttgtgtgtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 116 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 117 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 118 - protein, 1035 aa, synthetic construct
MPEQERQITAREGASRKILSKLSLPTRAWEPAMKKSFAFDNVGYEGGLDGLGPSSQPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 119 - protein, 1035 aa, synthetic construct
MPEQERQITAREGASRKILSKLSLPTRAWEPAMKKSFAFDNVGYEGGLDGLGPSSQPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVRVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQKYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAAPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 120 - protein, 1037 aa, synthetic construct
MAAPEQERQITAREGASRKILSKLSLPTRAWEPAMKKSFAFDNVGYEGGLDGLGPSSQPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 121 - protein, 1101 aa, synthetic construct
MPEQERQITAREGASRKILSKLSLPTRAWEPAMKKSFAFDNVGYEGGLDGLGPSSQVAPDGAEGSGTDHRSSSSHSPGSPPRNQVQGTCSKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLVSLFFCIPVMGLMIYMMVMDHHFATLHHNQNMSKEEMINLHSSMFLERQILPGLSVMNLLSFLLCVPVQFFGGWYFYIQAYKALKHKTANMDVLIVLATTIAFAYSLIILLVAMYERAKVNPITFFDTPPMLFVFIALGRWLEHIAKGKTSEALAKLISLQATEATIVTLDSDNILLSEEQVDVELVQRGDIIKVVPGGKFPVDGRVIEGHSMVDESLITGEAMPVAKKPGSTVIAGSINQNGSLLICATHVGADTTLSQIVKLVEEAQTSKAPIQQFADKLSGYFVPFIVFVSIATLLVWIVIGFLNFEIVETYFPGYNRSISRTETIIRFAFQASITVLCIACPCSLGLATPTAVMVGTGVGAQNGILIKGGEPLEMAHKVKVVVFDKTGTITHGTPVVNQVKVLTESNRISHHKILAIVGTAESNSEHPLGTAITKYCKQELDTETLGTCIDFQVVPGCGISCKVTNIEGLLHKNNWNIEDNNIKNASLVQIDASNEQSSTSSSMIIDAQISNALNAQQYKVLIGNREWMIRNGLVINNDVNDFMTEHERKGRTAVLVAVDDELCGLIAIADTVKPEAELAIHILKSMGLEVVLMTGDNSKTARSIASQVGITKVFAEVLPSHKVAKVKQLQEEGKRVAMVGDGINDSPALAMANVGIAIGTGTDVAIEAADVVLIRNDLLDVVASIDLSRKTVKRIRINFVFALIYNLVGIPIAAGVFMPIGLVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 122 - protein, 1103 aa, synthetic construct
MAAPEQERQITAREGASRKILSKLSLPTRAWEPAMKKSFAFDNVGYEGGLDGLGPSSQVAPDGAEGSGTDHRSSSSHSPGSPPRNQVQGTCSKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLVSLFFCIPVMGLMIYMMVMDHHFATLHHNQNMSKEEMINLHSSMFLERQILPGLSVMNLLSFLLCVPVQFFGGWYFYIQAYKALKHKTANMDVLIVLATTIAFAYSLIILLVAMYERAKVNPITFFDTPPMLFVFIALGRWLEHIAKGKTSEALAKLISLQATEATIVTLDSDNILLSEEQVDVELVQRGDIIKVVPGGKFPVDGRVIEGHSMVDESLITGEAMPVAKKPGSTVIAGSINQNGSLLICATHVGADTTLSQIVKLVEEAQTSKAPIQQFADKLSGYFVPFIVFVSIATLLVWIVIGFLNFEIVETYFPGYNRSISRTETIIRFAFQASITVLCIACPCSLGLATPTAVMVGTGVGAQNGILIKGGEPLEMAHKVKVVVFDKTGTITHGTPVVNQVKVLTESNRISHHKILAIVGTAESNSEHPLGTAITKYCKQELDTETLGTCIDFQVVPGCGISCKVTNIEGLLHKNNWNIEDNNIKNASLVQIDASNEQSSTSSSMIIDAQISNALNAQQYKVLIGNREWMIRNGLVINNDVNDFMTEHERKGRTAVLVAVDDELCGLIAIADTVKPEAELAIHILKSMGLEVVLMTGDNSKTARSIASQVGITKVFAEVLPSHKVAKVKQLQEEGKRVAMVGDGINDSPALAMANVGIAIGTGTDVAIEAADVVLIRNDLLDVVASIDLSRKTVKRIRINFVFALIYNLVGIPIAAGVFMPIGLVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 123 - protein, 1164 aa, synthetic construct
MPEQERQITAREGASRKILSKLSLPTRAWEPAMKKSFAFDNVGYEGGLDGLGPSSQVATTLIAIAGMTCASCVHSIEGMISQLEGVQQISVSLAEGTATVLYNPSVISPEELRAAIEDMGFEASVVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 124 - protein, 1166 aa, synthetic construct
MAAPEQERQITAREGASRKILSKLSLPTRAWEPAMKKSFAFDNVGYEGGLDGLGPSSQVATTLIAIAGMTCASCVHSIEGMISQLEGVQQISVSLAEGTATVLYNPSVISPEELRAAIEDMGFEASVVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 125 - protein, 1168 aa, synthetic construct
MPEQERQITAREGASRKILSKLSLPTRAWEPAMKKSFAFDNVGYEGGLDGLGPSSQVATSTVRILGMTCQSCVKSIEDRISNLKGIISMKVSLEQGSATVKYVPSVVCLQQVCHQIGDMGFEASIPDGAEGSGTDHRSSSSHSPGSPPRNQVQGTCSKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLVSLFFCIPVMGLMIYMMVMDHHFATLHHNQNMSKEEMINLHSSMFLERQILPGLSVMNLLSFLLCVPVQFFGGWYFYIQAYKALKHKTANMDVLIVLATTIAFAYSLIILLVAMYERAKVNPITFFDTPPMLFVFIALGRWLEHIAKGKTSEALAKLISLQATEATIVTLDSDNILLSEEQVDVELVQRGDIIKVVPGGKFPVDGRVIEGHSMVDESLITGEAMPVAKKPGSTVIAGSINQNGSLLICATHVGADTTLSQIVKLVEEAQTSKAPIQQFADKLSGYFVPFIVFVSIATLLVWIVIGFLNFEIVETYFPGYNRSISRTETIIRFAFQASITVLCIACPCSLGLATPTAVMVGTGVGAQNGILIKGGEPLEMAHKVKVVVFDKTGTITHGTPVVNQVKVLTESNRISHHKILAIVGTAESNSEHPLGTAITKYCKQELDTETLGTCIDFQVVPGCGISCKVTNIEGLLHKNNWNIEDNNIKNASLVQIDASNEQSSTSSSMIIDAQISNALNAQQYKVLIGNREWMIRNGLVINNDVNDFMTEHERKGRTAVLVAVDDELCGLIAIADTVKPEAELAIHILKSMGLEVVLMTGDNSKTARSIASQVGITKVFAEVLPSHKVAKVKQLQEEGKRVAMVGDGINDSPALAMANVGIAIGTGTDVAIEAADVVLIRNDLLDVVASIDLSRKTVKRIRINFVFALIYNLVGIPIAAGVFMPIGLVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 126 - protein, 1196 aa, synthetic construct
MPEQERQITAREGASRKILSKLSLPTRAWEPAMKKSFAFDNVGYEGGLDGLGPSSQVAPDGAEGSGTDHRSSSSHSPGSPPRNQVQGTCSTTLIAIAGMTCASCVHSIEGMISQLEGVQQISVSLAEGTATVLYNPSVISPEELRAAIEDMGFEASVVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 127 - protein, 1230 aa, synthetic construct
MPEQERQITAREGASRKILSKLSLPTRAWEPAMKKSFAFDNVGYEGGLDGLGPSSQVAPDGAEGSGTDHRSSSSHSPGSPPRNQVQGTCSTTLIAIAGMTCASCVHSIEGMISQLEGVQQISVSLAEGTATVLYNPSVISPEELRAAIEDMGFEASVVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLVSLFFCIPVMGLMIYMMVMDHHFATLHHNQNMSKEEMINLHSSMFLERQILPGLSVMNLLSFLLCVPVQFFGGWYFYIQAYKALKHKTANMDVLIVLATTIAFAYSLIILLVAMYERAKVNPITFFDTPPMLFVFIALGRWLEHIAKGKTSEALAKLISLQATEATIVTLDSDNILLSEEQVDVELVQRGDIIKVVPGGKFPVDGRVIEGHSMVDESLITGEAMPVAKKPGSTVIAGSINQNGSLLICATHVGADTTLSQIVKLVEEAQTSKAPIQQFADKLSGYFVPFIVFVSIATLLVWIVIGFLNFEIVETYFPGYNRSISRTETIIRFAFQASITVLCIACPCSLGLATPTAVMVGTGVGAQNGILIKGGEPLEMAHKVKVVVFDKTGTITHGTPVVNQVKVLTESNRISHHKILAIVGTAESNSEHPLGTAITKYCKQELDTETLGTCIDFQVVPGCGISCKVTNIEGLLHKNNWNIEDNNIKNASLVQIDASNEQSSTSSSMIIDAQISNALNAQQYKVLIGNREWMIRNGLVINNDVNDFMTEHERKGRTAVLVAVDDELCGLIAIADTVKPEAELAIHILKSMGLEVVLMTGDNSKTARSIASQVGITKVFAEVLPSHKVAKVKQLQEEGKRVAMVGDGINDSPALAMANVGIAIGTGTDVAIEAADVVLIRNDLLDVVASIDLSRKTVKRIRINFVFALIYNLVGIPIAAGVFMPIGLVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 128 - protein, 1465 aa, Homo sapiens [sequence omitted]

(19) United States
(12) Patent Application Publication
(10) Pub. No.: US 2026/0124322 A1
(43) Pub. Date: May 7, 2026

(54) ATP7B GENE THERAPY

(71) Applicant: MeiraGTx UK II Limited, London (GB)

(72) Inventors: Alexandria Forbes, New York, NY (US); Josefa Sullivan, New York, NY (US); Edgar Hernandez, New York, NY (US); Matthew During, Rowayton, CT (US); Ce Feng Liu, New York, NY (US)

(21) Appl. No.: 19/117,721

(22) PCT Filed: Oct. 11, 2023
(86) PCT No.: PCT/IB2023/060247

§ 371 Date: Apr. 2, 2025

Related U.S. Application Data
(60) Provisional application No. 63/379,113, filed on Oct. 11, 2022.

(51) Int. Cl. A61K 48/00; A61K 38/43; A61P 3/02; C12N 15/86

(52) U.S. Cl. CPC A61K 48/0058; A61K 38/43; A61K 48/0066; A61P 3/02; C12N 15/86; C12N 2750/14143

(57) ABSTRACT

Provided herein are improved expression constructs for the expression of ATP7B, vectors, and pharmaceutical compositions comprising such constructs. Also provided are methods of treating disease, including, but not limited to, Wilson's Disease.

### CROSS-REFERENCE TO RELATED APPLICATIONS

This application claims the benefit under 35 U.S.C. § 119(e) of the earlier filing date of U.S. Provisional Patent No. 63/379,113, filed Oct. 11, 2022, which is hereby incorporated by reference in its entirety.

### REFERENCE TO AN ELECTRONIC SEQUENCE LISTING

The contents of the electronic sequence listing (SeqList-162027.53176.xml; Size: 479,210 bytes; and Date of Creation: Oct. 10, 2023) is herein incorporated by reference in its entirety.

### FIELD

The present disclosure relates generally to the field of molecular biology and medicine. More particularly, the methods and compositions herein are useful for treating Wilson's Disease.

### BACKGROUND

Wilson's Disease (WD) is caused by autosomal recessive, loss-of-function mutations in the ATPase copper transporting beta (ATP7B) gene which lead to pathological accumulation of copper in the liver, brain, and other tissues. Symptoms of WD include Parkinson's Disease like neurological defects (including dystonia/bradykinesia) and hepatologic defects associated with cirrhosis. The prevalence of Wilson's Disease is 1 in ˜30,000 people.

ATP7B is a transmembrane copper ion transporter. When the cellular copper concentration increases, ATP7B translocates to the lysosome and pumps copper into vesicles so that the copper can be excreted via the bile duct in the liver. Due it is large size (1465 amino acids), ATP7B is too large to be effectively packaged into adeno-associated virus (AAV), making ATP7B gene therapy difficult.

Accordingly, enhanced ATP7B expression constructs for the treatment of Wilson's Disease are urgently needed.

### SUMMARY

Provided herein are improved expression constructs for the expression of ATP7B, vectors, and pharmaceutical compositions comprising such constructs, and methods of using such constructs, vectors, and pharmaceutical compositions.

In one aspect, provided is an expression construct comprising:
(a) a promoter;
(b) a sequence encoding (ATPase Copper Transporting Beta) ATP7B, operatively linked to the promoter; and
(c) a polyadenylation signal.

In some embodiments, the promoter comprises a sequence that is at least 80% identical to a sequence selected from the group consisting of SEQ ID NOs:4-33. In some embodiments, the promoter comprises a sequence that is at least 90% identical to a sequence selected from the group consisting of SEQ ID NOs:4-33. In some embodiments, the promoter comprises a sequence that is at least 95% identical to a sequence selected from the group consisting of SEQ ID NOs:4-33. In some embodiments, the promoter comprises a sequence selected from the group consisting of SEQ ID NOs:4-33. In some embodiments, the promoter comprises a sequence that is at least 80% identical to any one of SEQ ID NO:4, SEQ ID NO:10, or SEQ ID NO:11. In some embodiments, the promoter comprises a sequence that is at least 90% identical to any one of SEQ ID NOs:4, 10, or 11. In some embodiments, the promoter comprises a sequence that is at least 95% identical to any one of SEQ ID NOs:4, 10, or 11. In some embodiments, the promoter comprises any one of SEQ ID NOs:4, 10, or 11.

In some embodiments, the sequence encoding ATP7B is codon-optimized. In some embodiments, the sequence encoding ATP7B comprises a sequence that is at least 80% identical to any one of SEQ ID NOs:35-48. In some embodiments, the sequence encoding ATP7B comprises a sequence that is at least 90% identical to any one of SEQ ID NOs:35-48. In some embodiments, the sequence encoding ATP7B comprises a sequence that is at least 95% identical to any one of SEQ ID NOs:35-48. In some embodiments, the sequence encoding ATP7B comprises a sequence selected from the group consisting of SEQ ID NOs:35-48. In some embodiments, the sequence encoding ATP7B comprises a sequence that is at least 80% identical to SEQ ID NO:39 or SEQ ID NO:41. In some embodiments, the sequence encoding ATP7B comprises a sequence that is at least 90% identical to SEQ ID NO:39 or SEQ ID NO:41. In some embodiments, the sequence encoding ATP7B comprises a sequence that is at least 95% identical to SEQ ID NO:39 or SEQ ID NO:41. In some embodiments, the sequence encoding ATP7B comprises SEQ ID NO:39 or SEQ ID NO:41.

In some embodiments, the sequence encoding ATP7B encodes a protein comprising a sequence that is at least 80% identical to any one of SEQ ID NOs:118-128. In some embodiments, the sequence encoding ATP7B encodes a protein comprising a sequence that is at least 90% identical to any one of SEQ ID NOs:118-128. In some embodiments, the sequence encoding ATP7B encodes a protein comprising a sequence that is at least 95% identical to any one of SEQ ID NOs:118-128. In some embodiments, the sequence encoding ATP7B encodes a protein comprising any one of SEQ ID NOs:118-128. In some embodiments, the sequence encoding ATP7B encodes a protein comprising a sequence that is at least 80% identical to SEQ ID NO:118 or SEQ ID NO:123. In some embodiments, the sequence encoding ATP7B encodes a protein comprising a sequence that is at least 90% identical to SEQ ID NO:118 or SEQ ID NO:123. In some embodiments, the sequence encoding ATP7B encodes a protein comprising a sequence that is at least 95% identical to SEQ ID NO:118 or SEQ ID NO:123. In some embodiments, the sequence encoding ATP7B encodes a protein comprising SEQ ID NO:118 or SEQ ID NO:123.

In some embodiments, the expression construct further comprises a post-transcriptional regulatory element. In some embodiments, the post-transcriptional regulatory element comprises a sequence that is at least 80% identical to SEQ ID NO:49 or SEQ ID NO:50. In some embodiments, the post-transcriptional regulatory element comprises a sequence that is at least 90% identical to SEQ ID NO:49 or SEQ ID NO:50. In some embodiments, the post-transcriptional regulatory element comprises a sequence that is at least 95% identical to SEQ ID NO:49 or SEQ ID NO:50. In some embodiments, the post-transcriptional regulatory element comprises SEQ ID NO:49 or SEQ ID NO:50. In some embodiments, the post-transcriptional regulatory element comprises a sequence that is at least 80% identical to SEQ ID NO:49. In some embodiments, the post-transcriptional regulatory element comprises a sequence that is at least 90% identical to SEQ ID NO:49. In some embodiments, the post-transcriptional regulatory element comprises a sequence that is at least 95% identical to SEQ ID NO:49. In one embodiment, the post-transcriptional regulatory element comprises SEQ ID NO:49.

In some embodiments, the polyadenylation signal comprises a sequence that is at least 80% identical to SEQ ID NO:51 or SEQ ID NO:52. In some embodiments, the polyadenylation signal comprises a sequence that is at least 90% identical to SEQ ID NO:51 or SEQ ID NO:52. In some embodiments, the polyadenylation signal comprises a sequence that is at least 95% identical to SEQ ID NO:51 or SEQ ID NO:52. In some embodiments, the polyadenylation signal comprises SEQ ID NO:51 or SEQ ID NO:52. In some embodiments, the polyadenylation signal comprises a sequence that is at least 80% identical to SEQ ID NO:51. In some embodiments, the polyadenylation signal comprises a sequence that is at least 90% identical to SEQ ID NO:51. In some embodiments, the polyadenylation signal comprises a sequence that is at least 95% identical to SEQ ID NO:51. In one embodiment, the polyadenylation signal comprises SEQ ID NO:51.

In some embodiments, the expression construct further comprises a miRNA (miR) binding site (miRBS). In some embodiments, the miRBS comprises a sequence that is at least 80% identical to SEQ ID NO:53. In some embodiments, the miRBS comprises a sequence that is at least 90% identical to SEQ ID NO:53. In some embodiments, the miRBS comprises a sequence that is at least 95% identical to SEQ ID NO:53. In one embodiment, the miRBS comprises SEQ ID NO:53.

In some embodiments, provided is a construct comprising:
(a) a promoter comprising a sequence that is at least 80% identical to SEQ ID NO:11;
(b) a sequence encoding ATP7B, operatively linked to the promoter, comprising a sequence that is at least 80% identical to SEQ ID NO:41;
(c) a post-transcriptional regulatory element comprising a sequence that is at least 80% identical to SEQ ID NO:49; and
(d) a polyadenylation signal comprising a sequence that is at least 80% identical to SEQ ID NO:51.

In some embodiments, provided is a construct comprising:
(a) a promoter comprising a sequence that is at least 90% identical to SEQ ID NO:11;
(b) a sequence encoding ATP7B, operatively linked to the promoter, comprising a sequence that is at least 90% identical to SEQ ID NO:41;
(c) a post-transcriptional regulatory element comprising a sequence that is at least 90% identical to SEQ ID NO:49; and
(d) a polyadenylation signal comprising a sequence that is at least 90% identical to SEQ ID NO:51.

In some embodiments, provided is a construct comprising:
(a) a promoter comprising a sequence that is at least 95% identical to SEQ ID NO:11;
(b) a sequence encoding ATP7B, operatively linked to the promoter, comprising a sequence that is at least 95% identical to SEQ ID NO:41;
(c) a post-transcriptional regulatory element comprising a sequence that is at least 95% identical to SEQ ID NO:49; and
(d) a polyadenylation signal comprising a sequence that is at least 95% identical to SEQ ID NO:51.

In some embodiments, provided is a construct comprising:
(a) a promoter comprising SEQ ID NO:11;
(b) a sequence encoding ATP7B, operatively linked to the promoter, comprising SEQ ID NO:41;
(c) a post-transcriptional regulatory element comprising SEQ ID NO:49; and
(d) a polyadenylation signal comprising SEQ ID NO:51.

In some embodiments, provided is a construct comprising:
(a) a promoter comprising a sequence that is at least 80% identical to SEQ ID NO:10;
(b) a sequence encoding ATP7B, operatively linked to the promoter, comprising a sequence that is at least 80% identical to SEQ ID NO:39;
(c) a miRBS comprising a sequence that is at least 80% identical to SEQ ID NO:53;
(d) a post-transcriptional regulatory element comprising a sequence that is at least 80% identical to SEQ ID NO:49; and (e) a polyadenylation signal comprising a sequence that is at least 80% identical to SEQ ID NO:51.

In some embodiments, provided is a construct comprising:
(a) a promoter comprising a sequence that is at least 90% identical to SEQ ID NO:10;
(b) a sequence encoding ATP7B, operatively linked to the promoter, comprising a sequence that is at least 90% identical to SEQ ID NO:39;
(c) a miRBS comprising a sequence that is at least 90% identical to SEQ ID NO:53;
(d) a post-transcriptional regulatory element comprising a sequence that is at least 90% identical to SEQ ID NO:49; and
(e) a polyadenylation signal comprising a sequence that is at least 90% identical to SEQ ID NO:51.

In some embodiments, provided is a construct comprising:
(a) a promoter comprising a sequence that is at least 95% identical to SEQ ID NO:10;
(b) a sequence encoding ATP7B, operatively linked to the promoter, comprising a sequence that is at least 95% identical to SEQ ID NO:39;
(c) a miRBS comprising a sequence that is at least 95% identical to SEQ ID NO:53; and
(d) a post-transcriptional regulatory element comprising a sequence that is at least 95% identical to SEQ ID NO:49; and
(e) a polyadenylation signal comprising a sequence that is at least 95% identical to SEQ ID NO:51.

In some embodiments, provided is a construct comprising:
(a) a promoter comprising SEQ ID NO:10;
(b) a sequence encoding ATP7B, operatively linked to the promoter, comprising SEQ ID NO:39;
(c) a miRBS comprising SEQ ID NO:53;
(d) a post-transcriptional regulatory element comprising SEQ ID NO:49; and (e) a polyadenylation signal comprising SEQ ID NO:51.

In some embodiments, provided is a construct comprising:
(a) a promoter comprising a sequence that is at least 80% identical to SEQ ID NO:4;
(b) a sequence encoding ATP7B, operatively linked to the promoter, comprising a sequence that is at least 80% identical to SEQ ID NO:41;
(c) a post-transcriptional regulatory element comprising a sequence that is at least 80% identical to SEQ ID NO:49; and
(d) a polyadenylation signal comprising a sequence that is at least 80% identical to SEQ ID NO:51.

In some embodiments, provided is a construct comprising:
(a) a promoter comprising a sequence that is at least 90% identical to SEQ ID NO:4;
(b) a sequence encoding ATP7B, operatively linked to the promoter, comprising a sequence that is at least 90% identical to SEQ ID NO:41;
(c) a post-transcriptional regulatory element comprising a sequence that is at least 90% identical to SEQ ID NO:49; and
(d) a polyadenylation signal comprising a sequence that is at least 90% identical to SEQ ID NO:51.

In some embodiments, provided is a construct comprising:
(a) a promoter comprising a sequence that is at least 95% identical to SEQ ID NO:4;
(b) a sequence encoding ATP7B, operatively linked to the promoter, comprising a sequence that is at least 95% identical to SEQ ID NO:41;
(c) a post-transcriptional regulatory element comprising a sequence that is at least 95% identical to SEQ ID NO:49; and
(d) a polyadenylation signal comprising a sequence that is at least 95% identical to SEQ ID NO:51.

In some embodiments, provided is a construct comprising:
(a) a promoter comprising SEQ ID NO:4;
(b) a sequence encoding ATP7B, operatively linked to the promoter, comprising SEQ ID NO:41;
(c) a post-transcriptional regulatory element SEQ ID NO:49; and
(d) a polyadenylation signal comprising SEQ ID NO:51.

In one aspect, provided is a vector comprising an expression construct disclosed herein. In one embodiment, the vector is a viral vector. In one embodiment, the vector is an AAV vector. In one aspect, provided is a vector comprising a nucleic acid sequence comprising (i) an expression construct disclosed herein and (ii) one or more inverted terminal repeats (ITR). In one embodiment, the nucleic acid sequence comprises a 5′ ITR and a 3′ ITR. In one embodiment, the 5′ ITR and the 3′ ITR are derived from adeno-associated virus (AAV) serotype AAV2. In some embodiments, the sequence of the 5′ ITR is at least 80% identical to SEQ ID NO:116. In some embodiments, the sequence of the 5′ ITR is at least 90% identical to SEQ ID NO:116. In some embodiments, the sequence of the 5′ ITR is at least 95% identical to SEQ ID NO:116. In some embodiments, the sequence of the 5′ ITR comprises SEQ ID NO:116. In some embodiments, the sequence of the 3′ ITR is at least 80% identical to SEQ ID NO: 117. In some embodiments, the sequence of the 3′ ITR is at least 90% identical to SEQ ID NO:117. In some embodiments, the sequence of the 3′ ITR is at least 95% identical to SEQ ID NO: 117. In some embodiments, the sequence of the 3′ ITR comprises SEQ ID NO:117.

In one aspect, provided is a vector comprising an expression construct, wherein the vector comprises a sequence that is least 80% identical to any one of SEQ ID NOs:54-115. In some embodiments, the vector comprises a sequence that is least 90% identical to any one of SEQ ID NOs:54-115. In some embodiments, the vector comprises a sequence that is least 95% identical to any one of SEQ ID NOs:54-115. In some embodiments, the vector comprises any one of SEQ ID NOs:54-115. In some embodiments, the vector comprises a sequence that is least 80% identical to any one of SEQ ID NOs:65, 73, or 92. In some embodiments, the vector comprises a sequence that is least 90% identical to any one of SEQ ID NOs:65, 73, or 92. In some embodiments, the vector comprises a sequence that is least 95% identical to SEQ ID NOs:65, 73, or 92. In some embodiments, the vector comprises SEQ ID NOs:65, 73, or 92. In some embodiments, the vector comprises a capsid comprising or derived from AAV7m8, AAV9, AAV2-retro, or AAVrh.10.

In one aspect, provided is a cell comprising an expression construct or a vector disclosed herein.

In one aspect, provided is a pharmaceutical composition comprising (i) an expression construct or a vector disclosed herein and (ii) a pharmaceutically acceptable carrier.

In one aspect, provided is a method of increasing ATP7B activity in a subject in need thereof, the method comprising administering to the subject an expression construct, a vector or a pharmaceutical composition disclosed herein. In one aspect, provided is a method of increasing copper secretion in a subject in need thereof, the method comprising administering to the subject an expression construct, a vector or a pharmaceutical composition disclosed herein. In one aspect, provided is a method of treating a condition caused by a deficiency or dysfunction of ATP7B in a subject in need thereof, the method comprising administering to the subject an expression construct, a vector or a pharmaceutical composition disclosed herein. In one aspect, provided is a method of treating Wilson's Disease in a subject in need thereof, the method comprising administering to the subject an expression construct, a vector or a pharmaceutical composition disclosed herein. In one aspect, provided is a method of reducing dystonia or bradykinesia in a subject suffering from Wilson's Disease, the method comprising administering to the subject an expression construct, a vector or a pharmaceutical composition disclosed herein. In one aspect, provided is a method of reducing the incidence of leukopenia or anemia in a subject suffering from Wilson's Disease, the method comprising administering to the subject an expression construct, a vector or a pharmaceutical composition disclosed herein. In one aspect, provided is a method of reducing the incidence of cirrhosis in a subject suffering from Wilson's Disease, the method comprising administering to the subject an expression construct, a vector or a pharmaceutical composition disclosed herein. In some embodiments the subject is a human.

### BRIEF DESCRIPTION OF THE FIGURES

FIG. 1 illustrates the structure of ATP7B.

FIGS. 2A and 2B illustrate how the activity of liver-specific promoters disclosed herein in human Huh7 cells was compared with commonly used reference promoters. FIG. 2A. Plasmid used in dual-reporter flow-based assay. FIG. 2B. Relative protein expression (compared to expression using control promoter CAG) for different liver specific promoters. Arrows indicate that the L15 and L13 promoters drove particularly high expression of miniATP7B in-vitro among the promoters tested. AAT, LP1, HLP, TBG, and HCB served as additional reference promoters.

FIGS. 3A, 3B, and 3C illustrate how the expression, and the associated copper ion efflux function of the ATP7B minigenes, were examined. FIG. 3A. A schematic of the copper responsive reporter construct used for experiments shown in FIGS. 3B and 3C. The reporter in this construct is driven by a copper-responsive promoter. The amount of reporter expressed is a direct reflection of the amount of copper ions found intracellularly, which is in turn is modulated by the copper pumping activity of ATP7B. ATP7B minigenes were tested for copper ion efflux activity in ATP7B knockout (KO) cells using a copper reporter in combination with flow cytometry. mClover3 fluorescence increased upon treatment with copper sulfate. FIG. 3B. The increase in mClover3 fluorescence was attenuated by expression of either full-length (FL) ATP7B or ATP7B minigenes (TG1-TG3) TG1=miniATP7Bv_v2. TG2=miniATP7B-s1co. TG3=miniATP7AB. TG=transgene. MFI=Median Fluorescence Intensity. Labeling of the graph is the same as in FIG. 3C. FIG. 3C. Potency of transgenes (TG) was assessed by measuring copper reporter activity at different concentrations of ATP7B transgene plasmids used for transfection. Ctrl=control.

FIGS. 4A, 4B, 4C, and 4D illustrate that a combination of a variety of different genetic elements including promoters, a TISU sequence, 3′ UTR elements (miRNA sites, WPRE, polyAs) and ATP7B minigenes can increase ATP7B expression. FIG. 4A. Western blot showing ATP7B expression in HEK293 ATP7B−/− cells that had been transfected with the indicated expression constructs (see Table 7). AAT-miniATP7B-sPolyA served as a reference construct (REF). FIGS. 4B, 4C, and 4D. Western blot showing ATP7B expression in Huh7 cells (FIG. 4B), primary murine hepatocytes (FIG. 4C), and primary human hepatocytes (FIG. 4D), respectively, that had been transduced with AAV8 particles containing the indicated expression constructs. In construct A39 (labeled AAT-A12), the L15 promoter in A12 was replaced with the standard AAT promoter. Human ACTB (beta-actin) served as a control.

FIGS. 5A, 5B, 5C, and 5D illustrate that over-expression of ATP7B minigenes rescues alanine aminotransferase (ALT) and splenomegaly phenotypes in ATP7B−/− mice. AAV8 (5e12 GC/kg i.v.) were injected into ATP7B−/− male mice at 7 weeks of age. ALT activity in serum was measured 8 weeks after injection. After 12 weeks, organs were weighed and expression was measured in the liver. FIGS. 5A and 5B. Western blot (FIG. 5A) and quantification (FIG. 5B) of ATP7B protein expression in the liver of ATP7B mice after 12 weeks. FIG. 5C. ALT activity in the serum of ATP7B mice after 8 weeks. FIG. 5D. Spleen weight of ATP7B mice after 12 weeks.

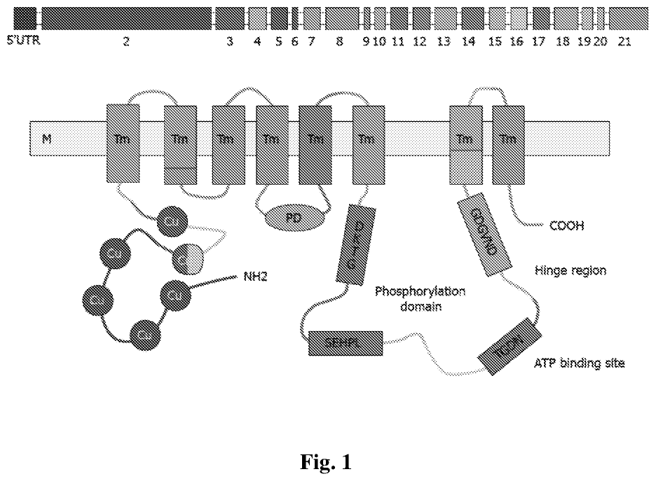

### DETAILED DESCRIPTION

Provided herein are improved expression constructs for the expression of ATP7B, vectors, and pharmaceutical compositions comprising such constructs, and methods of using such constructs, vectors, and pharmaceutical compositions. In some embodiments, the expression constructs disclosed herein show enhanced ATP7B expression, allowing lower MOIs (multiplicity of infection) of virus to be used clinically, which in turn can improve safety outcomes in patients and lower manufacturing hurdles including cost. In embodiments, the expression constructs disclosed herein show improved liver function and decreased immune response.

### Expression Constructs

In one aspect, provided is an expression construct comprising:
(a) a promoter;
(b) a sequence encoding ATP7B, operatively linked to the promoter; and
(c) a polyadenylation signal.

As used herein, “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

In one aspect, provided is an expression construct comprising:
(a) a promoter;
(b) a sequence encoding ATP7B, operatively linked to the promoter;
(c) a miRNA binding site (miRBS);
(d) a post-transcriptional regulatory element; and/or
(e) a polyadenylation signal.

In one aspect, provided is an expression construct comprising from 5′ to 3′:
(a) a promoter;
(b) a sequence encoding ATP7B, operatively linked to the promoter;
(c) a miRNA binding site (miRBS);
(d) a post-transcriptional regulatory element; and
(e) a polyadenylation signal.

In one aspect, provided is an expression construct:
(a) a promoter;
(b) a sequence encoding ATP7B, operatively linked to the promoter;
(c) a post-transcriptional regulatory element; and/or
(d) a polyadenylation signal.

In one aspect, provided is an expression construct from 5′ to 3′:
(a) a promoter;
(b) a sequence encoding ATP7B, operatively linked to the promoter;
(c) a post-transcriptional regulatory element; and
(d) a polyadenylation signal.

As used herein, the term “from 5′ to 3′” refers to the order of the specific genetic elements in a nucleic sequence. In some embodiments, the specific genetic elements are linked to one another by way of a linker nucleic acid (e.g., an intervening non-coding nucleic acid). In some embodiments, the specific genetic elements are linked to one another with no intervening nucleotides present. In some embodiments, some of the specific genetic elements are linked to one another by way of a linker nucleic acid, while other are linked to one another with no intervening nucleotides present.

In some embodiments, the expression construct comprises a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs: 4-33. In some embodiments, the expression construct comprises a promoter sequence comprising any one of SEQ ID NOs: 4-33. In some embodiments, the expression construct comprises a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NO:4, SEQ ID NO:10, or SEQ ID NO:11. some embodiments, the expression construct comprises a promoter sequence comprising any one of SEQ ID NO:4, SEQ ID NO:10, or SEQ ID NO:11.

In some embodiments, the expression construct comprises a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:4. In some embodiments, the expression construct comprises a promoter sequence comprising SEQ ID NO:4.

In some embodiments, the expression construct comprises a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:10. In some embodiments, the expression construct comprises a promoter sequence comprising SEQ ID NO:10.

In some embodiments, the expression construct comprises a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11. In some embodiments, the expression construct comprises a promoter sequence comprising SEQ ID NO:11.

In some embodiments, the expression construct comprises a Kozak sequence for initiating protein translation. In some embodiments, the expression construct comprises a Translation Initiator of Short 5′ UTR (TISU) sequence for initiating protein translation. See, e.g., Elfakess et al., Nucleic Acids Res. 2011 Sep. 1; 39(17):7598-609.

In some embodiments, the expression construct comprises a sequence encoding ATP7B wherein the ATP7B-encoding sequence is codon-optimized.

In some embodiments, the expression construct comprises a sequence encoding ATP7B, wherein the ATP7B-encoding sequence is at least 80%, at least 85%, at least 90%, at least 91%, at least 92%, at least 93%, at least 94%, at least 95%, at least 96%, at least 97%, at least 98%, or at least 99% identical to any one of SEQ ID NOs:35-48. In some embodiments, the expression construct comprises any one of SEQ ID NOs:35-48.

In some embodiments, the expression construct comprises a sequence encoding ATP7B, wherein the ATP7B-encoding sequence is at least 80%, at least 85%, at least 90%, at least 91%, at least 92%, at least 93%, at least 94%, at least 95%, at least 96%, at least 97%, at least 98%, or at least 99% identical to SEQ ID NO:39 or SEQ ID NO:41. In some embodiments, the expression construct to SEQ ID NO:39 or SEQ ID NO:41.

In some embodiments, the expression construct comprises a sequence encoding ATP7B, wherein the sequence encodes a ATP7B protein comprising a sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs:118-128. In some embodiments, the expression construct comprises a sequence encoding ATP7B, wherein the sequence encodes a ATP7B protein comprising any one of SEQ ID NOs:118-128.

In some embodiments, the expression construct comprises a sequence encoding ATP7B, wherein the sequence encodes a ATP7B protein comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:118 or SEQ ID NO:123. In some embodiments, the expression construct comprises a sequence encoding ATP7B, wherein the sequence encodes a ATP7B protein comprising SEQ ID NO:118 or SEQ ID NO:123.

In some embodiments, the expression construct comprises a miRNA binding site (miRBS). In embodiments, the miRBS comprises one or more (e.g., 1 to 6) binding sites for a microRNA. In embodiments, the miRBS comprises one or more binding sites for miR142 (TCCATAAAGTAGGAAACACTACA; SEQ ID NO: 6). In embodiments, the miRBS comprises 4 binding sites for miR142. In embodiments where the miRBS comprises 2 or more binding sites for miR142, the miR142 binding sites may be joined by a nucleotide linker of 1 to 10 nucleotides.

In some embodiments, the expression construct comprises a miRBS comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:53. In some embodiments, the expression construct comprises a miRBS comprising SEQ ID NO:53.

In some embodiments, the expression construct comprises a post-transcriptional regulatory element. In some embodiments, the expression construct comprises a woodchuck hepatitis virus post-transcriptional regulatory element (WPRE).

In some embodiments, the expression construct comprises a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:49 or SEQ ID NO:50. In some embodiments, the expression construct comprises a post-transcriptional regulatory element comprising SEQ ID NO:49 or SEQ ID NO:50.

In some embodiments, the expression construct comprises a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:49. In some embodiments, the expression construct comprises a post-transcriptional regulatory element comprising SEQ ID NO:49.

In some embodiments, the expression construct comprises a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:51 or SEQ ID NO:52. In some embodiments, the expression construct comprises a polyadenylation signal comprising SEQ ID NO:51 or SEQ ID NO:52.

In some embodiments, the expression construct comprises a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:51. In some embodiments, the expression construct comprises a polyadenylation signal comprising SEQ ID NO:51.

In one embodiment, the expression construct comprises:
(a) a promoter comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:11;
(b) a sequence encoding ATP7B, operatively linked to the promoter, comprising a sequence that is at least 80%, at least 85%, at least 90%, at least 91%, at least 92%, at least 93%, at least 94%, at least 95%, at least 96%, at least 97%, at least 98%, or at least 99% identical to SEQ ID NO:41;
(c) a post-transcriptional regulatory element comprising a sequence that is least 80%, at least 85%, at least 90%, at least 91%, at least 92%, at least 93%, at least 94%, at least 95%, at least 96%, at least 97%, at least 98%, or at least 99% identical to SEQ ID NO:49; and
(d) a polyadenylation signal comprising a sequence that is least 80%, at least 85%, at least 90%, at least 91%, at least 92%, at least 93%, at least 94%, at least 95%, at least 96%, at least 97%, at least 98%, or at least 99% identical to SEQ ID NO:51.

In one embodiment, the expression construct comprises:
(a) a promoter comprising SEQ ID NO:11;
(b) a sequence encoding ATP7B, operatively linked to the promoter, comprising SEQ ID NO:41;
(c) a post-transcriptional regulatory element comprising SEQ ID NO:49; and
(d) a polyadenylation signal comprising SEQ ID NO:51.

In one embodiment, the expression construct comprises:
(a) a promoter comprising a sequence that is least 80%, at least 85%, at least 90%, at least 91%, at least 92%, at least 93%, at least 94%, at least 95%, at least 96%, at least 97%, at least 98%, or at least 99% identical to SEQ ID NO:10;
(b) a sequence encoding ATP7B, operatively linked to the promoter, comprising a sequence that is least 80%, at least 85%, at least 90%, at least 91%, at least 92%, at least 93%, at least 94%, at least 95%, at least 96%, at least 97%, at least 98%, or at least 99% identical to SEQ ID NO:39;
(c) a miRBS comprising a sequence that is least 80%, at least 85%, at least 90%, at least 91%, at least 92%, at least 93%, at least 94%, at least 95%, at least 96%, at least 97%, at least 98%, or at least 99% identical to SEQ ID NO:53;
(d) a post-transcriptional regulatory element comprising a sequence that is least 80%, at least 85%, at least 90%, at least 91%, at least 92%, at least 93%, at least 94%, at least 95%, at least 96%, at least 97%, at least 98%, or at least 99% identical to SEQ ID NO:49; and
(e) a polyadenylation signal comprising a sequence that is least 80%, at least 85%, at least 90%, at least 91%, at least 92%, at least 93%, at least 94%, at least 95%, at least 96%, at least 97%, at least 98%, or at least 99% identical to SEQ ID NO:51.

In one embodiment, the expression construct comprises:
(a) a promoter comprising SEQ ID NO:10;
(b) a sequence encoding ATP7B, operatively linked to the promoter, comprising SEQ ID NO:39;
(c) a miRBS comprising SEQ ID NO:53;
(d) a post-transcriptional regulatory element comprising SEQ ID NO:49; and
(e) a polyadenylation signal comprising SEQ ID NO:51.

In one embodiment, the expression construct comprises:
(a) a promoter comprising a sequence that is least 80%, at least 85%, at least 90%, at least 91%, at least 92%, at least 93%, at least 94%, at least 95%, at least 96%, at least 97%, at least 98%, or at least 99% identical to SEQ ID NO:4;
(b) a sequence encoding ATP7B, operatively linked to the promoter, comprising a sequence that is least 80%, at least 85%, at least 90%, at least 91%, at least 92%, at least 93%, at least 94%, at least 95%, at least 96%, at least 97%, at least 98%, or at least 99% identical to SEQ ID NO:41;
(c) a post-transcriptional regulatory element comprising a sequence that is least 80%, at least 85%, at least 90%, at least 91%, at least 92%, at least 93%, at least 94%, at least 95%, at least 96%, at least 97%, at least 98%, or at least 99% identical to SEQ ID NO:49; and
(d) a polyadenylation signal comprising a sequence that is least 80%, at least 85%, at least 90%, at least 91%, at least 92%, at least 93%, at least 94%, at least 95%, at least 96%, at least 97%, at least 98%, or at least 99% identical to SEQ ID NO:51.

In one embodiment, the expression construct comprises:
(a) a promoter comprising SEQ ID NO:4;
(b) a sequence encoding ATP7B, operatively linked to the promoter, comprising SEQ ID NO:41;
(c) a post-transcriptional regulatory element SEQ ID NO:49; and
(d) a polyadenylation signal comprising SEQ ID NO:51.

Provided herein are the expression constructs of Table 7. Provided herein are the expression constructs comprising one or more of the genetic elements shown in Table 7.

### Vectors

In one aspect, provided are recombinant vectors and their use for the introduction of a transgene or an expression construct into a cell. In some embodiments, the recombinant vectors comprise recombinant DNA constructs that include additional DNA elements, including DNA segments that provide for the replication of the DNA in a host cell and expression of the target gene in target cells at appropriate levels. The ordinarily skilled artisan appreciates that expression control sequences (promoters, enhancers, and the like) are selected based on their ability to promote expression of the target gene in the target cell.

“Vector,” as used herein, means a vehicle that comprises a polynucleotide to be delivered into a host cell, either in vitro or in vivo. Non-limiting examples of vectors include a recombinant plasmid, yeast artificial chromosome (YAC), mini chromosome, DNA mini-circle, or a virus (including virus derived sequences). A vector may also refer to a virion comprising a nucleic acid to be delivered into a host cell, either in vitro or in vivo. In some embodiments, a vector refers to a virion comprising a recombinant viral genome, wherein the viral genome comprises one or more ITRs and a transgene.

In one embodiment, the recombinant vector is a viral vector or a combination of multiple viral vectors. In one aspect, provided is a vector comprising any of the expression constructs disclosed herein.

### Viral Vectors

Viral vectors for the expression of a target gene in a target cell, tissue, or organism are known in the art and include, for example, an AAV vector, adenovirus vector, lentivirus vector, retrovirus vector, poxvirus vector, baculovirus vector, herpes simplex virus vector, vaccinia virus vector, or a synthetic virus vector (e.g., a chimeric virus, mosaic virus, or pseudotyped virus, and/or a virus that contains a foreign protein, synthetic polymer, nanoparticle, or small molecule).

### AAV Vectors

Adeno-associated viruses (AAV) are small, single-stranded DNA viruses which require helper virus to facilitate efficient replication. The 4.7 kb genome of AAV is characterized by two inverted terminal repeats (ITR) and two open reading frames which encode the Rep proteins and Cap proteins, respectively. The Rep reading frame encodes four proteins of molecular weight 78 kD, 68 kD, 52 kD, and 40 kD. These proteins function mainly in regulating AAV replication and rescue and integration of the AAV into a host cell's chromosomes. The Cap reading frame encodes three structural proteins of molecular weight 85 kD (VP1), 72 kD (VP2), and 61 kD (VP3), which form the virion capsid. More than 80% of total proteins in AAV virion comprise VP3. Flanking the rep and cap open reading frames at the 5′ and 3′ ends are about 145 bp long inverted terminal repeats (ITRs). The two ITRs are the only cis elements essential for AAV replication, rescue, packaging, and integration of the AAV genome. The entire rep and cap domains can be excised and replaced with a therapeutic or reporter transgene.

Recombinant adeno-associated virus “rAAV” vectors include any vector derived from any adeno-associated virus serotype. rAAV vectors can have one or more of the AAV wild-type genes deleted in whole or in part, preferably the Rep and/or Cap genes, but retain functional flanking ITR sequences.

In some embodiments, the viral vector is an rAAV virion, which comprises an rAAV genome and one or more capsid proteins. In some embodiments, the rAAV genome comprises an expression construct disclosed herein.

In some embodiments, the viral vector disclosed herein comprises a nucleic acid comprising an AAV 5′ ITR and 3′ ITR located 5′ and 3′ to sequence encoding ATP7B, respectively. However, in certain embodiments, it may be desirable for the nucleic acid to contain the 5′ ITR and 3′ ITR sequences arranged in tandem, e.g., 5′ to 3′ or a head-to-tail, or in another alternative configuration. In still other embodiments, it may be desirable for the nucleic acid to contain multiple copies of the ITRs or to have 5′ ITRs (or conversely, 3′ ITRs) located both 5′ and 3′ to the sequence encoding ATP7B. The ITRs sequences may be located immediately upstream and/or downstream of the heterologous molecule, or there may be intervening sequences. The ITRs need not be the wild-type nucleotide sequences, and may be altered (e.g., by the insertion, deletion, or substitution of nucleotides) so long as the sequences provide for functional rescue, replication, and packaging. The ITRs may be selected from AAV2, or from among the other AAV serotypes, as described herein.

In some embodiments, provided is a vector comprising a nucleic acid sequence comprising (i) an expression construct disclosed herein and (ii) one or more inverted terminal repeats (ITR). In one embodiment, the nucleic acid sequence comprises a 5′ ITR and a 3′ ITR. In one embodiment, the 5′ ITR and the 3′ ITR are derived from adeno-associated virus (AAV) serotype AAV2.

In one embodiment, the 5′ ITR sequence comprises a sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs:116-177. In one embodiment, the 5′ ITR sequence comprises any one of SEQ ID NOs:116-177.

In one embodiment, the 3′ ITR sequence comprises a sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs:178-239. In one embodiment, the 3′ ITR sequence comprises any one of SEQ ID NOs:178-239.

Provided herein is a vector comprising a nucleic acid sequence comprising a sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs:54-115. Provided herein is a vector comprising any one of SEQ ID NOs:54-115.

Provided herein is a vector comprising a nucleic acid sequence comprising a sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NO:65, SEQ ID NO:73, or SEQ ID NO:92. Provided herein is a vector comprising any one of SEQ ID NO:65, SEQ ID NO:73, or SEQ ID NO:92.

In some embodiments, the viral vector is an AAV vector, such as an AAV1 (i.e., an AAV containing AAV1 ITRs and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), or AAVrh.10 (i.e., an AAV containing AAVrh.10 ITRs and AAVrh.10 capsid proteins).

In some embodiments, the viral vector is a pseudotyped AAV vector, containing ITRs from one AAV serotype and capsid proteins from a different AAV serotype. In some embodiments, the pseudotyped AAV is AAV2/9 (i.e., an AAV containing AAV2 ITRs and AAV9 capsid proteins). In some embodiments, the pseudotyped AAV is AAV2/10 (i.e., an AAV containing AAV2 ITRs and AAV10 capsid proteins). In some embodiments, the pseudotyped AAV is AAV2/8 (i.e., an AAV containing AAV2 ITRs and AAV8 capsid proteins).

In some embodiments, the pseudotyped AAV is AAV2/7m8 (i.e., an AAV containing AAV2 ITRs and AAV7m8 capsid proteins).

In some embodiments, the AAV vector contains a recombinant capsid protein, such as a capsid protein containing a chimera of one or more of capsid proteins from AAV1, AAV2, AAV3, AAV4, AAV5, AAV6, AAV7, AAV8, AAV9, AAV10, AAVrh74, AAVrh.8, or AAVrh.10. In embodiments, the capsid is a variant AAV capsid such as the AAV2 variant rAAV2-retro (SEQ ID NO:44 from WO 2017/218842, incorporated herein by reference). In one embodiment, the capsid protein is derived from AAV8. In some embodiment, the capsid is derived from AAV-3B, AAV-S3, AAV3B-DE5, AAV-GT5, AAV-KP1, AAV-LK03, AAV-208 (AAVrh10/AAV8 hybrid, see Charbel et al., Assessment of tropism and effectiveness of new primate-derived hybrid recombinant AAV serotypes in the mouse and primate retina. PLoS One. 2013 Apr. 9; 8(4):e60361), AAV-Anc80, AAV-CMRI_30 (AAV2 with T503A and N596D mutations, see PCT publication WO02021/000,024), AAV2-N496D, AAV2-N582S, AAV-NP59 (see Paulk et al., Bioengineered AAV Capsids with Combined High Human Liver Transduction In Vivo and Unique Humoral Seroreactivity. Mol Ther. 2018 Jan. 3; 26(1):289-303), AAV-hu.T88 (see Chen et al., Molecular characterization of adeno-associated viruses infecting children. J Virol. 2005 December; 79(23):14781-92), AAV-hu.S17 (see Chen et al., 2005), AAV-2TT (see Tordo et al., A novel adeno-associated virus capsid with enhanced neurotropism corrects a lysosomal transmembrane enzyme deficiency. Brain. 2018 Jul. 1; 141(7):2014-2031), or AAV-2.htT88-MEAS (AAV2/hu.T88 mosaic).

### Other Viral Vectors

Other viral vectors include adenoviral (AV) vectors, for example, those based on human adenovirus type 2 and human adenovirus type 5 that have been made replication defective through deletions in the E1 and E3 regions. The transcriptional cassette can be inserted into the E1 region, yielding a recombinant E1/E3-deleted AV vector. Adenoviral vectors also include helper-dependent high-capacity adenoviral vectors (also known as high-capacity, “gutless” or “gutted” vectors), which do not contain viral coding sequences. These vectors contain the cis-acting elements needed for viral DNA replication and packaging, mainly the inverted terminal repeat sequences (ITR) and the packaging signal (CY). These helper-dependent AV vector genomes have the potential to carry from a few hundred base pairs up to approximately 36 kb of foreign DNA.

Alternatively, other systems such as lentiviral vectors can be used. Lentiviral-based systems can transduce nondividing as well as dividing cells making them useful for applications targeting, for examples, the nondividing cells of the CNS. Lentiviral vectors are derived from the human immunodeficiency virus and, like that virus, integrate into the host genome providing the potential for very long-term gene expression.

Polynucleotides, including plasmids, YACs, minichromosomes and minicircles, carrying the target gene containing the expression cassette can also be introduced into a cell or organism by nonviral vector systems using, for example, cationic lipids, polymers, or both as carriers. Conjugated poly-L-lysine (PLL) polymer and polyethylenimine (PEI) polymer systems can also be used to deliver the vector to cells. Other methods for delivering the vector to cells includes hydrodynamic injection and electroporation and use of ultrasound, both for cell culture and for organisms. For a review of viral and non-viral delivery systems for gene delivery see Nayerossadat, N. et al. (Adv Biomed Res. 2012; 1:27) incorporated herein by reference.

rAAV Virion Production

The rAAV virions disclosed herein may be constructed and produced using the materials and methods described herein, as well as those known to those of skill in the art. Such engineering methods used to construct any embodiment of this disclosure are known to those with skill in nucleic acid manipulation and include genetic engineering, recombinant engineering, and synthetic techniques. See, e.g., Sambrook et al, “Molecular Cloning. A Laboratory Manual”, 2d ed., Cold Spring Harbor Laboratory, New York (1989), and Ausubel et al., Current Protocols in Molecular Biology, John Wiley & Sons, New York, 1989); and International Patent Publication No. WO 95/13598. Further, methods suitable for producing a rAAV cassette in an adenoviral capsid have been described in U.S. Pat. Nos. 5,856,152 and 5,871,982.

Briefly, in order to package the rAAV genome into a rAAV virion, a host cell is used that contains sequences necessary to express AAV rep and AAV cap or functional fragments thereof as well as helper genes essential for AAV production. The AAV rep and cap sequences are obtained from an AAV source as identified herein. The AAV rep and cap sequences may be introduced into the host cell in any manner known to one in the art, including, without limitation, transfection, electroporation, liposome delivery, membrane fusion techniques, high velocity DNA-coated pellets, viral infection, and protoplast fusion. In one embodiment, the rep and cap sequences may be transfected into the host cell by one or more nucleic acid molecules and exist stably in the cell as an episome. In another embodiment, the rep and cap sequences are stably integrated into the genome of the cell. Another embodiment has the rep and cap sequences transiently expressed in the host cell. For example, a useful nucleic acid molecule for such transfection comprises, from 5′ to 3′, a promoter, an optional spacer interposed between the promoter and the start site of the rep gene sequence, an AAV rep gene sequence, and an AAV cap gene sequence.

The rep and cap sequences, along with their expression control sequences, may be supplied on a single vector, or each sequence may be supplied on its own vector. Preferably, the rep and cap sequences are supplied on the same vector. Alternatively, the rep and cap sequences may be supplied on a vector that contains other DNA sequences that are to be introduced into the host cells. Preferably, the promoter used in this construct may be any suitable constitutive, inducible or native promoters known to one of skill in the art. The molecule providing the rep and cap proteins may be in any form which transfers these components to the host cell. Desirably, this molecule is in the form of a plasmid, which may contain other non-viral sequences, such as those for marker genes. This molecule does not contain the AAV ITRs and generally does not contain the AAV packaging sequences. To avoid the occurrence of homologous recombination, other virus sequences, particularly those of adenovirus, are avoided in this plasmid. This plasmid is desirably constructed so that it may be stably transfected into a cell.

Although the molecule providing rep and cap may be transiently transfected into the host cell, it is preferred that the host cell be stably transformed with sequences necessary to express functional rep/cap proteins in the host cell, e.g., as an episome or by integration into the chromosome of the host cell. Depending upon the promoter controlling expression of such stably transfected host cell, the rep/cap proteins may be transiently expressed (e.g., through use of an inducible promoter).

The methods employed for constructing embodiments of this disclosure are conventional genetic engineering or recombinant engineering techniques such as those described in the references above. For example, the rAAV may be produced utilizing a triple transfection method using either the calcium phosphate method (Clontech) or Effectene reagent (Qiagen, Valencia, Calif.), according to manufacturer's instructions. See, also, Herzog et al, 1999, Nature Medic., 5(1):56-63, for the method used in the following examples, employing the plasmid with the transgene, a helper plasmid containing AAV rep and cap, and a plasmid supplying adenovirus helper functions of E2A, E4Orf6 and VA. While this specification provides illustrative examples of specific constructs, using the information provided herein, one of skill in the art may select and design other suitable constructs, using a choice of spacers, promoters, and other elements, including at least one translational start and stop signal, and the optional addition of polyadenylation sites.

The rAAV virions are then produced by culturing a host cell containing a rAAV virus as described herein which contains a rAAV genome to be packaged into a rAAV virion, an AAV rep sequence and an AAV cap sequence under the control of regulatory sequences directing expression thereof. Suitable viral helper genes, e.g., adenovirus E2A, E4Orf6 and VA, among other possible helper genes, may be provided to the culture in a variety of ways known to the art, preferably on a separate plasmid. Thereafter, the recombinant AAV virion which directs expression of the ATP7B transgene is isolated from the cell or cell culture in the absence of contaminating helper virus or wildtype AAV.

Expression of the ATP7B transgene may be measured in ways known in the art. For example, a target cell may be infected in vitro, and the number of copies of the transgene in the cell monitored by Southern blotting or quantitative polymerase chain reaction (PCR). The level of RNA expression may be monitored by Northern blotting or quantitative reverse transcriptase (RT)-PCR; and the level of protein expression may be monitored by Western blotting, immunohistochemistry, enzyme-linked immunosorbent assay (ELISA), radioimmunoassay (RIA) or by the specific methods detailed below in the Examples.

### Pharmaceutical Composition

Provided herein are pharmaceutical compositions comprising any of the vectors disclosed herein and a pharmaceutically acceptable excipient.

In some embodiments, the rAAV comprising the gene encoding ATP7B is assessed for contamination by conventional methods and then formulated into a pharmaceutical composition suitable for storage and/or administration to a patient.

Formulations of the vectors disclosed herein involve the use of a pharmaceutically and/or physiologically acceptable vehicle or carrier, particularly one suitable for subretinal injection, such as buffered saline or other buffers, e.g., HEPES, to maintain pH at appropriate physiological levels.

The vector of the disclosure can be formulated into pharmaceutical compositions. These compositions may comprise, in addition to the vector, a pharmaceutically and/or physiologically acceptable excipient, carrier, buffer, stabilizer, antioxidants, preservative, or other additives well known to those skilled in the art. Such materials should be non-toxic and should not interfere with the efficacy of the active ingredient. The precise nature of the carrier or other material may be determined by the skilled person according to the route of administration. The pharmaceutical composition is typically in liquid form. Liquid pharmaceutical compositions generally include a liquid carrier such as water, petroleum, animal or vegetable oils, mineral oil or synthetic oil. Additional carriers are provided in International Patent Publication No. WO 00/15822, incorporated herein by reference. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used. In some cases, Ringer's Injection, Lactated Ringer's Injection, or Hartmann's solution is used. Preservatives, stabilizers, buffers, antioxidants and/or other additives may be included, as required. For delayed release, the vector may be included in a pharmaceutical composition which is formulated for slow release, such as in microcapsules formed from biocompatible polymers or in liposomal carrier systems according to methods known in the art.

If the vector is to be stored long-term, it may be frozen in the presence of glycerol.

### Methods of Treatment

Provided herein are methods of treating a disease in a subject in need thereof using expression constructs, vectors, and pharmaceutical compositions disclosed herein.

In some embodiments, the subject is a mammal. The term “mammal” as used herein is intended to include, but is not limited to, humans, laboratory animals, domestic pets, and farm animals. Mammals, include, but are not limited to, a human or non-human mammal, such as a bovine, equine, canine, ovine, or feline, etc. Individuals and patients are also subjects herein.

The terms “treat,” “treated,” “treating,” or “treatment” as used herein refer to therapeutic treatment, wherein the object is to slow down (lessen) an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of one or more symptoms of the condition, disorder or disease state; and remission (whether partial or total), or enhancement or improvement of the condition, disorder or disease. Treatment includes eliciting a clinically significant response without excessive levels of side effects. Treatment also includes prolonging survival as compared to expected survival if not receiving treatment.

The terms “prevent”, “prevention”, and the like refer to acting prior to overt disease or disorder onset, to prevent the disease or disorder from developing or to minimize the extent of the disease or disorder or slow its course of development.

Provided herein is a method of increasing ATP7B activity in a subject in need thereof, the method comprising administering to the subject a vector or a pharmaceutical composition disclosed herein.

Provided herein is a method of increasing copper secretion in a subject in need thereof, the method comprising administering to the subject a vector or a pharmaceutical composition disclosed herein.

Provided herein is a method of treating or preventing a condition caused by a deficiency or dysfunction of ATP7B in a subject in need thereof, the method comprising administering to the subject a vector or a pharmaceutical composition disclosed herein. Provided herein is a vector or a pharmaceutical composition disclosed herein for use in treating or preventing a condition caused by a deficiency or dysfunction of ATP7B in a subject in need thereof. Provided herein is the use of a vector in the manufacture of a medicament for treating or preventing a condition caused by a deficiency or dysfunction of ATP7B in a subject in need thereof.

Provided herein is a method of treating or preventing Wilson's Disease in a subject in need thereof, the method comprising administering to the subject a vector or a pharmaceutical composition disclosed herein. Provided herein is a vector or a pharmaceutical composition disclosed herein for use in treating or preventing Wilson's Disease in a subject in need thereof. Provided herein is a vector or a pharmaceutical composition disclosed herein in the manufacture of a medicament for treating or preventing Wilson's Disease in a subject in need thereof.

Provided herein is a method of treating or preventing dystonia or bradykinesia in a subject suffering from Wilson's Disease in a subject in need thereof, the method comprising administering to the subject a vector or a pharmaceutical composition disclosed herein. Provided herein is a vector or a pharmaceutical composition disclosed herein for use in treating or preventing dystonia or bradykinesia in a subject suffering from Wilson's Disease in a subject in need thereof. Provided herein is the use of a vector in the manufacture of a medicament for treating or preventing dystonia or bradykinesia in a subject suffering from Wilson's Disease in a subject in need thereof.

Provided herein is a method of reducing the incidence of leukopenia or anemia in a subject suffering from Wilson's Disease in a subject in need thereof, the method comprising administering to the subject a vector or a pharmaceutical composition disclosed herein. Provided herein is a vector or a pharmaceutical composition disclosed herein for use in reducing the incidence of leukopenia or anemia in a subject suffering from Wilson's Disease in a subject in need thereof. Provided herein is the use of a vector in the manufacture of a medicament for reducing the incidence of leukopenia or anemia in a subject suffering from Wilson's Disease in a subject in need thereof.

Provided herein is a method of reducing the incidence of cirrhosis in a subject suffering from Wilson's Disease in a subject in need thereof, the method comprising administering to the subject a vector or a pharmaceutical composition disclosed herein. Provided herein is a vector or a pharmaceutical composition disclosed herein for use in reducing the incidence of cirrhosis in a subject suffering from Wilson's Disease in a subject in need thereof. Provided herein is the use of a vector in the manufacture of a medicament for reducing the incidence of cirrhosis in a subject suffering from Wilson's Disease in a subject in need thereof.

In some embodiments, treatment refers to increased survival (e.g., survival time). For example, treatment can result in an increased life expectancy of a patient. In some embodiments, treatment results in an increased life expectancy of a patient by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200% or more, as compared to the average life expectancy of one or more control individuals with Wilson's Disease without treatment. In some embodiments, treatment results in an increased life expectancy of a patient by more than about 6 months, about 7 months, about 8 months, about 9 months, about 10 months, about 11 months, about 12 months, about 2 years, about 3 years, about 4 years, about 5 years, about 6 years, about 7 years, about 8 years, about 9 years, about 10 years or more, as compared to the average life expectancy of one or more control individuals with Wilson's Disease without treatment. In some embodiments, treatment results in long term survival of a patient. As used herein, the term “long term survival” refers to a survival time or life expectancy longer than about 40 years, 45 years, 50 years, 55 years, 60 years, or longer.

### Combination Therapy

In some embodiments, the expression construct or vector described herein is administered to a subject in combination with one or more additional therapies to treat Wilson's Disease. In embodiments, the expression construct or vector is administered in combination with a chelating agent. In embodiments, the expression construct or vector can be administered in combination with penicillamine (such as Cuprimine®, Depen®), trientine (such as Syprine®), zinc acetate (Galzin®).

In some embodiments, combined administration of the expression construct or vector and a second agent results in an improvement in Wilson's Disease or a symptom thereof to an extent that is greater than one produced by either the expression construct or vector or the second agent alone. The difference between the combined effect and the effect of each agent alone can be a statistically significant difference.

In some embodiments, combined administration of the expression construct or vector and a second agent allows administration of the second agent at a reduced dose, at a reduced number of doses, and/or at a reduced frequency of dosage compared to a standard dosing regimen approved for the second agent.

### Routes and Methods of Administration

Methods of administration include, but are not limited to, intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. The mode of administration is left to the discretion of the practitioner.

In some instances, the expression construct or vector described herein is administered locally. This can be achieved, for example, by local infusion during surgery, topical application (e.g., in a cream or lotion), by injection, by means of a catheter, by means of a suppository or enema, or by means of an implant, said implant being of a porous, non-porous, or gelatinous material, including membranes, such as sialastic membranes, or fibers. In some situations, the expression construct or vector described herein is introduced into the central nervous system, circulatory system or gastrointestinal tract by any suitable route, including intraventricular injection, intrathecal injection, paraspinal injection, epidural injection, enema, and by injection adjacent to a peripheral nerve.

The compositions described herein can be administered as single administrations or as multiple administrations. Such compositions can be administered at regular intervals, depending on the nature, severity and extent of the subject's condition. In some embodiments, a therapeutically effective amount of the expression construct or vector is administered intrathecally periodically at regular intervals (e.g., once every year, once every six months, once every five months, once every three months, bimonthly (once every two months), monthly (once every month), biweekly (once every two weeks), or weekly).

The amount of the expression construct or vector described herein that is effective for treating disease can be determined using standard clinical techniques known to those with skill in the art. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner.

An effective amount of an rAAV carrying a nucleic acid sequence encoding ATP7B under the control of the promoter may, for example, range between about 1×10⁹ to 1×10¹³ to about 1.5×10¹⁷ genome particles. A “genome particle” is defined herein as an AAV capsid that contains a single stranded DNA molecule that can be quantified with a sequence specific method (such as real-time PCR). In some embodiments, about 1×10¹³ to about 1.5×10¹⁷ viral genomes are used for systemic delivery. In some embodiments, about 1×10¹¹ viral genomes are used per animal. Still other dosages in these ranges may be selected by the attending physician.

It is to be understood that for any particular subject, specific dosage regimens can be adjusted over time according to the individual need and the professional judgment of the person administering or supervising the administration of the expression construct of vector and that dosage ranges set forth herein are exemplary only and are not intended to limit the scope or practice of the claimed disclosure.

In some embodiments, it may be desirable to administer multiple “booster” dosages of a pharmaceutical compositions disclosed herein. For example, depending upon the duration of the transgene within the target cell, one may deliver booster dosages at 6 month intervals, or yearly following the first administration. Other similar tests may be used to determine the status of the treated subject over time. Selection of the appropriate tests may be made by the attending physician.

### Articles of Manufacture and Kits

Also provided are kits or articles of manufacture for use in the methods described herein. In aspects, the kits comprise the compositions described herein (e.g., compositions for delivery of a ATP7B encoding transgene) in suitable packaging. Suitable packaging for compositions (such as ocular compositions for injection) described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

Also provided are kits comprising the compositions described herein. These kits may further comprise instruction(s) on methods of using the composition, such as uses described herein. The kits described herein may further include other materials desirable from a commercial and user standpoint, including buffers, diluents, filters, needles, syringes, and package inserts with instructions for performing the administration of the composition or performing any methods described herein. For example, in some embodiments, the kit comprises an rAAV for the expression of a ATP7B encoding transgene in target cells, a pharmaceutically acceptable carrier suitable for injection, and one or more of: a buffer, a diluent, a filter, a needle, a syringe, and a package insert with instructions for performing the injections.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

It is to be understood that this invention is not limited to the particular molecules, compositions, methodologies, or protocols described, as these may vary. Any methods and materials similar or equivalent to those described herein can be used in the practice or testing of embodiments of the present invention. It is further to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

All other referenced patents and applications are incorporated herein by reference in their entirety. Furthermore, where a definition or use of a term in a reference, which is incorporated by reference herein is inconsistent or contrary to the definition of that term provided herein, the definition of that term provided herein applies and the definition of that term in the reference does not apply.

To facilitate a better understanding of the present invention, the following examples of specific embodiments are given. The following examples should not be read to limit or define the entire scope of the invention.

### EXAMPLES

### Example 1: Methods for Examples 1-5

### Cell Culture

Huh-7, AML-12, and HEK 293 immortalized cell lines were maintained 37° C. in 5% CO₂. Cells were transiently transfected with plasmids using BC Transfection Reagent. After 72 hours, the cell lysates were collected for protein analysis. After 20 passages, a new aliquot of cells was thawed and passaged twice before use for subsequent experiments.

### Primary Hepatocyte Cultures

Mouse and human hepatocyte cell lines from Gibco were thawed and placed in suspended medium followed by centrifugation at 100×g for 10 minutes at 4° C. Supernatants were aspirated, and cells were resuspended in maintenance medium (+10% FBS) followed by plating at 400,000 cells per well on a 24-well plate. Four hours post plating, the plate was gently tapped to loosen debris, followed by aspiration of the medium, and replacement with non-FBS containing maintenance medium. Cells were maintained at 37° C. in 5% CO₂. Every day, full media changes were performed to feed the cultures.

### Transduction

On day 1 and day 3 in vitro for immortalized and primary cell cultures respectively, AAV8 particles were added to the medium at a multiplicity of infection (MOI) of 50,000. Doxorubicin containing medium was added on to the cells to speed up transduction. Media was changed 72 hours later to non-doxorubicin containing medium according to standard protocol.

### Western Blotting

Cells were gently washed with PBS before addition of ice-cold lysis buffer (CST) containing nuclease, and protease and phosphatase inhibitors. Cells were lysed on the plate for 30 minutes on ice with agitation and then collected. Liver tissue samples (approximately 30 mg) were lysed in cold RIPA buffer containing nuclease, and protease and phosphatase inhibitors with two rounds of sonication. Samples were vortexed vigorously to ensure complete lysis and then pelleted at 20,000×g on a micro-centrifuge for 15 minutes at 4° C. The supernatant was collected, and protein concentration determined by BCA (bicinchoninic acid) assay. Protein was then normalized to a standard concentration using 4×LDS, 10×DTT, and lysis buffer. Samples were denatured at 75° C. for 10 minutes.

For analysis of ATP7B expression, 10 g of protein samples were separated on Bolt 4-12% Bis-Tris denaturing gels (Invitrogen, USA) using MOPS buffer. Gels were then transferred onto nitrocellulose membranes at constant 0.4 A for 2 hours. Membranes were probed with primary antibodies (ATP7B, GAPDH, CHOP, CD11b, beta-Actin) diluted in Licor TBS Blocking Buffer overnight at 4° C. Membranes were then washed with TBS-T and probed with fluorescently conjugated anti-Mouse or anti-Rabbit IgG secondary antibodies for one hour at room temperature. Membranes were imaged on the LICOR Odyssey imaging system. Protein quantities were normalized using either the beta-Actin or GAPDH control bands.

### Animal Studies

7-wk old male ATP7B^{-/-} mice backcrossed onto C57BL/6 were acquired from Baylor College of Medicine and bred by Charles River Laboratories. Mice were housed at five animals per cage on a 12-h light/dark cycle (lights on from 0700 to 1900 h) at constant temperature (23° C.) with ad libitum access to food and water. All studies were reviewed by the Institutional Animal Care and Use Committee (IACUC).

Adeno-associated virus (AAV) serotype 2/8 (5×10¹² GC/kg) was delivered via tail vein injection at 7 weeks of age. Mice were restrained and placed under a warming light to increase vasodilation and the tail vein was targeted using a 27G needle. After 8 weeks, blood was collected into BD serum microcontainers (#02-675-185) by nicking the lateral tail vein after dilating the blood vessel with a heat lamp. Samples were allowed to clot at room temperature for 30 minutes, then spun at 2,000×g for 10 minutes in a refrigerated centrifuge. ALT activity was then measured in the resulting serum according to the manufacturer's protocol (Bioassays #EALT-100). After 12 weeks, animals were euthanized and perfused with cold PBS. Organs were collected and snap-frozen in liquid nitrogen before preparing for molecular analysis.

### Example 2: Identification of Potent Promoters for the Expression of ATP7B

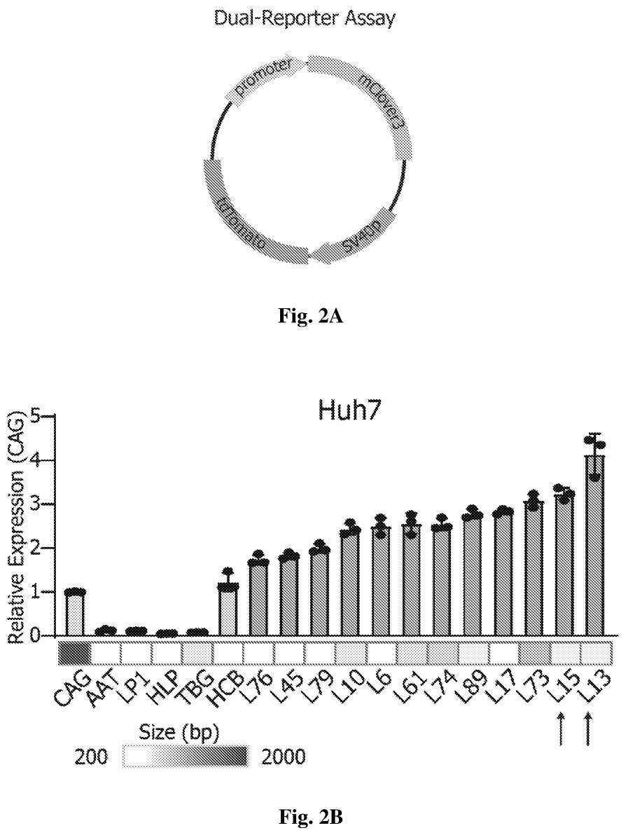

Promoters are integral components of a gene therapy that impact transgene expression level, timing, durability, and cell-type specificity. A library of tissue-specific and constitutive promoters was developed for the expression of ATP7B. See Tables 2-12. Provided herein are promoters that provided stronger expression in immortalized hepatocyte cells (Huh7, HepG2, AML-12) than commonly used reference promoters, including: CAG promoter (which consists of (1) the cytomegalovirus (CMV) early enhancer element, (2) the promoter, the first exon and the first intron of chicken beta-actin gene, and (3) the splice acceptor of the rabbit beta-globin gene), alpha-1-antitrypsin (AAT) promoter, and human thyroxine binding globulin (TBG) promoter, liver-specific promoter 1 (LP1), hybrid liver promoter (HLP), and hepatic combinatorial bundle promoter (HCB). The promoters in the promoter library were of equal or greater strength than CAG while also being significantly smaller. A subset of these liver-specific library promoters was selected to examine their use for the expression of miniATP7B minigenes. Using a dual-reporter flow-based assay in transfected human Huh7 cells, the activity of liver-specific promoters was compared to the activity of commonly used promoters (FIG. 2A).

In both human (Huh-7) and mouse (AML-12), both the L15 and L13 promoters drove the particularly high of miniATP7B in-vitro among the promoters tested (FIG. 2B) and Table 1.

### Example 3: Identification of Potent ATP7B Minigenes

In parallel, different variants of a miniATP7B gene were developed, see Tables 6 and 9.

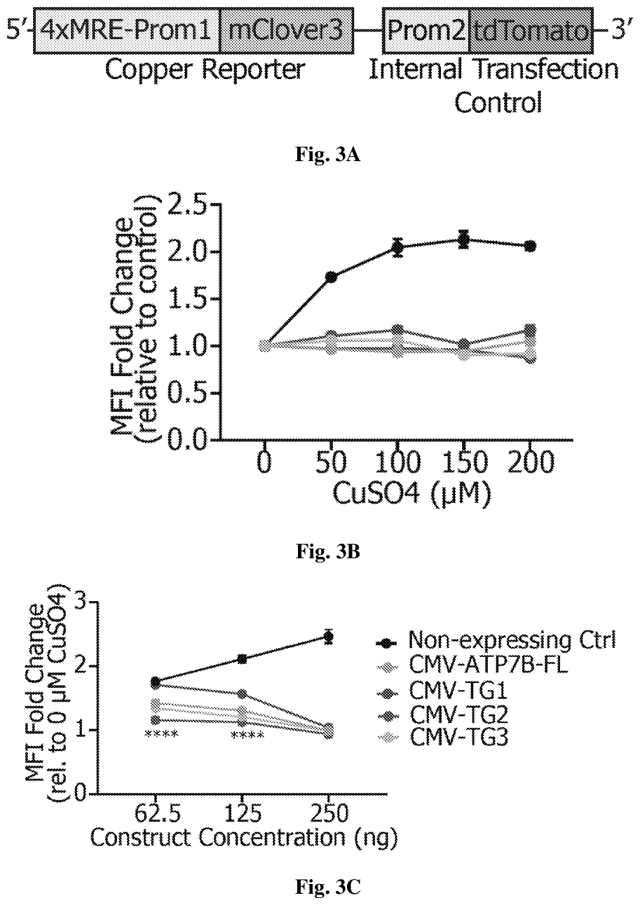

To assess ATP7B minigene activity, ATP7B knockout (KO) cells transfected with constructs comprising different transgenes were tested for copper pump activity using a copper reporter in combination with flow cytometry (FIG. 3A). mClover3 fluorescence increased upon treatment with copper sulfate. This increase was prevented by expression of either full-length ATP7B or ATP7B minigenes (FIG. 3B). The potency of transgenes was assessed by measuring copper reporter activity at decreasing concentrations of transgenes (FIG. 3C).

The minigenes were tested against wild-type ATP7B (full-length) in the copper reporting assay via transfection alongside a copper inducible mClover expressing construct (FIG. 3A). Function was assessed for each variant and found to be comparable to full-length wildtype ATP7B at all doses of copper sulfate tested. Potency was assessed for each variant by transfecting different concentrations of ATP7B and miniATP7B transgenes, followed by measuring the copper efflux activity via the copper reporter in combination with flow cytometry (FIG. 3C). miniATP7B variant TG2 (miniATP7B-s1co) showed improved copper pump activity at lower concentrations compared to ATP7B-FL, suggesting increased potency. Transgenes miniATP7B-slco (TG2) and miniATP7B-s1co3 encode a protein of the same amino acid sequence (SEQ ID NO:246). For the activity assays in FIGS. 3B and 3C, promoter and 3′ UTR elements were kept the same so that the difference in transgene activity could be directly compared.

### Example 4: ATP7B Expression Constructs

Selected promoter candidates were combined with selected miniATP7B transgene candidates and other regulatory elements (Tables 1 and 7). ATP7B expression was compared relative to a published reference cassette, AAT-miniATP7Bv (Murillo et al., Liver Expression of a MiniATP7B Gene Results in Long-Term Restoration of Copper Homeostasis in a Wilson Disease Model in Mice. Hepatology, 2019 July; 70(1):108-126) in different cellular models. This construct corresponds to construct A2 herein.

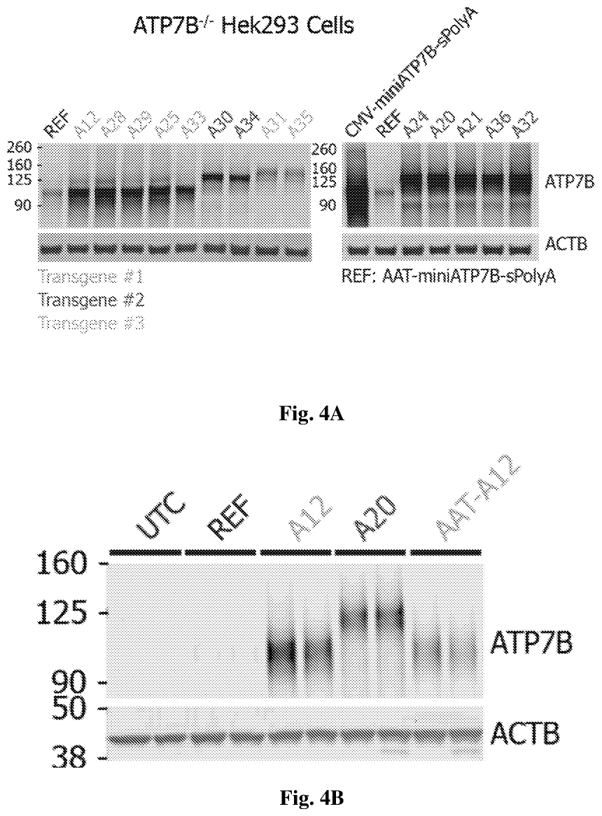

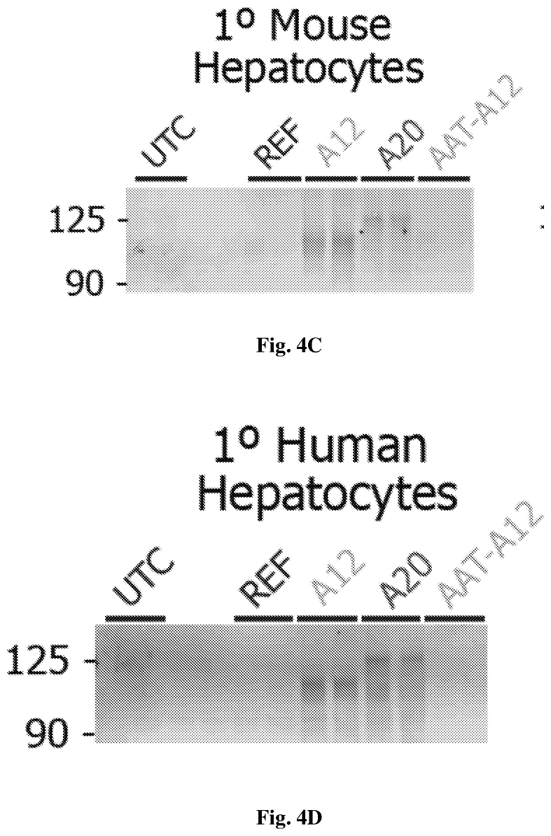

Constructs A12 and A20 showed particularly high ATP7B expression compared to the reference cassette in both AML12 and Huh-7 immortalized cell lines, as well in primary human and mouse hepatocytes following transduction with AAV8 (FIG. 4, Table 1). A comparison of constructs A12 to A39 highlights the importance of the promoter element for overall transgene expression. When the L15 promoter in A12 was replaced with the standard L1_v2 promoter (as in A39), transgene expression was significantly reduced in vitro (FIG. 4B).

TABLE 1
Summary of miniATP7B expression of the reference construct
and selected promoters across in vitro cellular models.

| | | | | | Transduction | | | |
|---|---|---|---|---|---|---|---|---|
| | | | | | | | Prim. | Prim. |
| | | | Transfection | | | | human | Mouse |
| ID | Promoter | Transgene | AML12 | HUH7 | AML12 | HUH7 | hepatocytes | hepatocytes |
| REF | L1_v2 | miniATP7Bv_v2 | 0.33 | 0.03 | 0.03 | 0.06 | 0.05 | 0.10 |
| (A2) | | | | | | | | |
| A12 | L15 | miniATP7Bv.co | 1.00 | 1.00 | 1.00 | 1.00 | 1.00 | 1.00 |
| A39 | L1_v2 | miniATP7Bv.co | 0.37 | 0.13 | 0.23 | 0.49 | 0.00 | 0.04 |
| A20 | L13_v2 | miniATP7B-s1co3 | 1.87 | 0.52 | 0.87 | 0.93 | 0.70 | 0.68 |

REF = AAT-miniATP7B-sPolyA disclosed in Murillo et al., 2019.
Prim. = primary.

### Example 5: ATP7B Expression In Vivo

Next, selected expression constructs A12, A20, and A39 were tested in an in vivo model (ATP7B knockout mice) to assess both function and rescue of phenotype using AAV8-mediated transduction.

Viruses were injected via tail vein of each mouse. Blood was collected at 8 weeks to assess ALT function. Organs were collected at 12 weeks post-injection to assess expression and spleen weight.

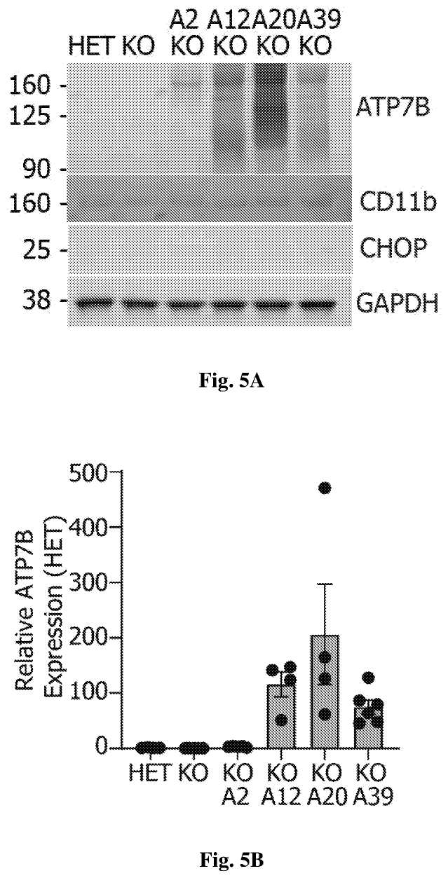

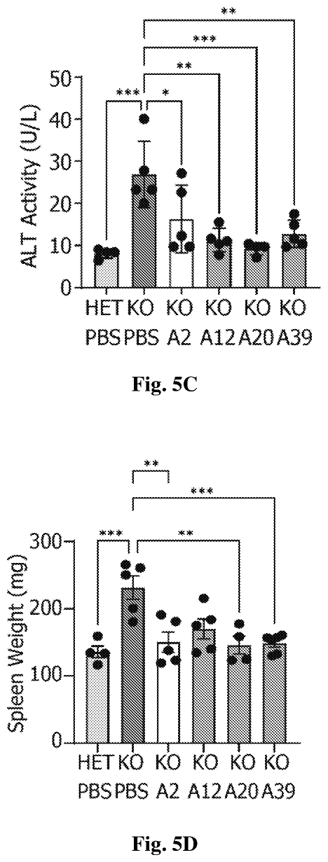

In accordance with the in vitro data, A12, A20, and A39 exhibited significantly higher protein expression than A2 in the liver of ATP7B mice (FIGS. 5A and 5B). A12 and A20 expressed 100- and 200-fold higher than endogenous ATP7B expression in heterozygous (HET) mice (FIG. 5B). HET mice are phenotypically the same as wild-type mice, but have 50% less ATP7B expression. Notably, this expression was not accompanied by expression of CD11b, an immune marker, or CHOP, an ER-stress marker (FIG. 5A). Moreover, miniATP7B expression was accompanied by the rescue of the Wilson's Disease phenotypes of increased alanine aminotransferase (ALT) activity due to liver damage and splenomegaly (FIGS. 5C and 5D).

### Overview of Sequences

An overview of sequences disclosed herein can be found in Tables 2-12.

TABLE 2
Overview of promoter sequences. See also Table 8.

| SEQ ID NO | Name | Alternative name |
|---|---|---|
| 1 | L1 | AAT |
| 2 | LP1 | |
| 3 | L68 | HLP |
| 4 | L1_v2 | |
| 5 | L10 | FGBcre1-mTTR (MPE_DPE) |
| 7 | L10_v2 | |
| 8 | L13 | |
| 9 | L13_TISU | |
| 10 | L13_v2 | |
| 11 | L15 | |
| 12 | L15_21 | |
| 13 | L15_21_TISU | |
| 14 | L15_TISU | |
| 15 | L16 | |
| 16 | L17 | SynEnh_v2-mTTR |
| 17 | L33 | |
| 18 | L45 | FTL |
| 19 | L57 | |
| 20 | L6 | ALBcre-mTTR |
| 21 | L61 | FTL6 |
| 22 | L73 | FTL10 |
| 23 | L74 | FTL11 |
| 24 | L75 | HCB-MVMi |
| 25 | L76 | |
| 26 | L79 | |
| 27 | L89 | |
| 28 | LP1v2 | |
| 29 | LP1v3 | |
| 30 | LP1v4 | |
| 31 | LP1v5 | |
| 32 | SynE_mTTR | |
| 33 | SynE_mTTR_TISU | |

TABLE 3
Overview of ATP7B minigenes sequences. See also Table 9.

| SEQ ID NO | Name |
|---|---|
| 34 | ATP7B_FL |
| 35 | miniATP7AB |
| 36 | miniATP7B-S1 |
| 37 | miniATP7B-s1co |
| 38 | miniATP7B-s1co2 |
| 39 | miniATP7B-s1co3 |
| 40 | miniATP7B-s6 |
| 41 | miniATP7Bv.co |
| 42 | miniATP7Bv_v2 |
| 43 | miniATP7B-WD4 |
| 44 | miniATP7B-WD5 |
| 45 | miniATP7B-WD6 |
| 46 | TISU-miniATP7B-s1co |
| 47 | TISU-miniATP7B-s1co3 |
| 48 | TISU-miniATP7Bv.co |

TABLE 4
Overview of additional genetic elements. See also Table 10.

| SEQ ID NO | Element |
|---|---|
| 49 | WPRE250 |
| 50 | WPRE3 |
| 51 | sPolyA |
| 52 | SV40 late pA |
| 53 | 4xmiR142 |
| 116 | 5′ ITR (used in A1-A62) |
| 117 | 3′ ITR (used in A1-A62) |

TABLE 5
Overview of expression construct sequences.
See also Tables 7 and 11.

| SEQ ID NO | Construct |
|---|---|
| 54 | A1 |
| 55 | A2 |
| 56 | A3 |
| 57 | A4 |
| 58 | A5 |
| 59 | A6 |
| 60 | A7 |
| 61 | A8 |
| 62 | A9 |
| 63 | A10 |
| 64 | A11 |
| 65 | A12 |
| 66 | A13 |
| 67 | A14 |
| 68 | A15 |
| 69 | A16 |
| 70 | A17 |
| 71 | A18 |
| 72 | A19 |
| 73 | A20 |
| 74 | A21 |
| 75 | A22 |
| 76 | A23 |
| 77 | A24 |
| 78 | A25 |
| 79 | A26 |
| 80 | A27 |
| 81 | A28 |
| 82 | A29 |
| 83 | A30 |
| 84 | A31 |
| 85 | A32 |
| 86 | A33 |
| 87 | A34 |
| 88 | A35 |
| 89 | A36 |
| 90 | A37 |
| 91 | A38 |
| 92 | A39 |
| 93 | A40 |
| 94 | A41 |
| 95 | A42 |
| 96 | A43 |
| 97 | A44 |
| 98 | A45 |
| 99 | A46 |
| 100 | A47 |
| 101 | A48 |
| 102 | A49 |
| 103 | A50 |
| 104 | A51 |
| 105 | A52 |
| 106 | A53 |
| 107 | A54 |
| 108 | A55 |
| 109 | A56 |
| 110 | A57 |
| 111 | A58 |
| 112 | A59 |
| 113 | A60 |
| 114 | A61 |
| 115 | A62 |

TABLE 6
Overview of protein sequences. See also Table 12.

| | Nucleic acid | Amino acid |
|---|---|---|
| Name | SEQ ID NO | SEQ ID NO |
| miniATP7Bv.co | 41 | 118 |
| miniATP7Bv_v2 | 42 | 119 |
| TISU-miniATP7Bv.co | 48 | 120 |
| miniATP7B-WD6 | 45 | 121 |
| miniATP7AB | 35 | 122 |
| miniATP7B-s1co3 | 39 | 123 |
| miniATP7B-s1co | 37 | 123 |
| miniATP7B-s1co2 | 38 | 123 |
| miniATP7B-S1 | 36 | 123 |
| TISU-miniATP7B-s1co | 46 | 124 |
| TISU-miniATP7B-s1co3 | 47 | 124 |
| miniATP7B-WD4 | 43 | 125 |
| miniATP7B-s6 | 40 | 126 |
| miniATP7B-WD5 | 44 | 127 |
| ATP7B_FL | 34 | 128 |

TABLE 7
Composition of expression constructs.

| | | | | MIR | | |
|---|---|---|---|---|---|---|
| | Construct | | | Sites | | polyA |
| Construct | (alternative name) | Promoter | Transgene | (3′) | WPRE | name |
| A1 | AAT-ATP7B | L1_v2 | ATP7B_FL | — | — | sPolyA |
| A2 | AAT-miniATP7Bv | L1_v2 | miniATP7Bv_{—} | — | — | sPolyA |
| | | | v2 | | | |
| A3 | SynE-mTTR- | SynE_mTTR | miniATP7Bv_{—} | — | — | sPolyA |
| | miniATP7Bv | | v2 | | | |
| A4 | SynE-mTTR-TISU- | SynE_mTTR_{—} | miniATP7AB | — | WPRE250 | sPolyA |
| | miniATP7AB-WPRE250 | TISU | | | | |
| A5 | SynE-mTTR- | SynE_mTTR | miniATP7B- | — | — | sPolyA |
| | miniATP7B.s1 | | S1 | | | |
| A6 | SynE-mTTR- | SynE_mTTR | miniATP7B- | — | — | sPolyA |
| | miniATP7B.s1co | | s1co | | | |
| A7 | SynE-mTTR- | SynE_mTTR | miniATP7B- | — | — | sPolyA |
| | miniATP7B.s6 | | s6 | | | |
| A8 | SynE-mTTR- | SynE_mTTR | miniATP7B- | — | — | sPolyA |
| | miniATP7B.WD4 | | WD4 | | | |
| A9 | SynE-mTTR- | SynE_mTTR | miniATP7B- | — | — | sPolyA |
| | miniATP7B.WD5 | | WD5 | | | |
| A10 | SynE-mTTR- | SynE_mTTR | miniATP7B- | — | — | sPolyA |
| | miniATP7B.WD6 | | WD6 | | | |
| A11 | SynE-mTTR(MD)- | L15 | miniATP7Bv.co | — | — | sPolyA |
| | miniATP7Bvco | | | | | |
| A12 | SynE-mTTR(MD)- | L15 | miniATP7Bv.co | — | WPRE250 | sPolyA |
| | miniATP7Bv.co- | | | | | |
| | WPRE250 | | | | | |
| A13 | FGBcre1-mTTR(MD)- | L10 | miniATP7Bv.co | — | — | sPolyA |
| | miniATP7Bv.co | | | | | |
| A14 | SERPINA1cre- | L13 | miniATP7Bv.co | — | — | sPolyA |
| | mTTR(MD)- | | | | | |
| | miniATP7Bv.co | | | | | |
| A15 | SERPINA1cre- | L13 | miniATP7B- | — | WPRE250 | sPolyA |
| | mTTR(MD)- | | WD6 | | | |
| | miniATP7AB-WPRE250 | | | | | |
| A16 | SERPINA1cre- | L13 | miniATP7B- | — | — | sPolyA |
| | mTTR(MD)- | | WD6 | | | |
| | miniATP7B.WD6 | | | | | |
| A17 | SERPINA1cre- | L13 | miniATP7B- | — | WPRE250 | sPolyA |
| | mTTR(MD)- | | s1co2 | | | |
| | miniATP7B.s1co2- | | | | | |
| | WPRE250 | | | | | |
| A18 | SynE-mTTR(MD)- | L15_21 | miniATP7Bv.co | — | WPRE250 | sPolyA |
| | miniATP7Bv.co- | | | | | |
| | WPRE250 | | | | | |
| A19 | SynE-mTTR(MD)- | L15_21_{—} | miniATP7Bv.co | — | WPRE250 | sPolyA |
| | miniATP7Bv.co- | TISU | | | | |
| | WPRE250 | | | | | |
| A20 | SERPINA1cre- | L13_v2 | miniATP7B- | 4xmiR142 | WPRE250 | sPolyA |
| | mTTR(MD)- | | s1co3 | | | |
| | miniATP7Bv.s1co3- | | | | | |
| | 4xmiR142-WPRE250 | | | | | |
| A21 | SERPINA1cre- | L13_v2 | miniATP7B- | 4xmiR142 | WPRE3 | sPolyA |
| | mTTR(MD)- | | s1co3 | | | |
| | ATP7B.s1co3-4xmiR142- | | | | | |
| | WPRE3 | | | | | |
| A22 | SERPINA1cre- | L13_v2 | miniATP7B- | 4xmiR142 | — | sPolyA |
| | mTTR(MD)- | | s1co3 | | | |
| | ATP7B.s1.co3-4xmiR142 | | | | | |
| A23 | SERPINA1cre- | L13_v2 | miniATP7B- | — | WPRE3 | sPolyA |
| | mTTR(MD)- | | s1co3 | | | |
| | ATP7B.s1co3-WPRE3 | | | | | |
| A24 | SERPINA1cre- | L13_v2 | miniATP7B- | — | — | sPolyA |
| | mTTR(MD)- | | s1co3 | | | |
| | ATP7B.s1co3 | | | | | |
| A25 | SERPINA1cre- | L13 | miniATP7Bv.co | — | WPRE250 | sPolyA |
| | mTTR(MD)- | | | | | |
| | miniATP7Bv.co- | | | | | |
| | WPRE250 | | | | | |
| A26 | SERPINA1cre- | L13 | miniATP7AB | — | WPRE250 | sPolyA |
| | mTTR(MD)- | | | | | |
| | miniATP7AB-WPRE250 | | | | | |
| A27 | SERPINA1cre- | L13 | miniATP7B- | — | WPRE250 | sPolyA |
| | mTTR(MD)- | | s1co | | | |
| | miniATP7Bv.s1co- | | | | | |
| | WPRE250 | | | | | |
| A28 | SynE-mTTR(MD)- | L15 | miniATP7Bv.co | — | WPRE3 | sPolyA |
| | miniATP7Bv.co-WPRE3 | | | | | |
| A29 | SynE-mTTR(MD)-TISU- | L15_TISU | TISU- | — | WPRE3 | sPolyA |
| | miniATP7Bv.co-WPRE3 | | miniATP7Bv.co | | | |
| A30 | SynE-mTTR(MD)-TISU- | L15_TISU | TISU- | — | WPRE3 | sPolyA |
| | miniATP7Bv.s1co- | | miniATP7B- | | | |
| | WPRE3 | | s1co | | | |
| A31 | SynE-mTTR(MD)-TISU- | L15_TISU | miniATP7AB | — | WPRE3 | sPolyA |
| | miniATP7AB-WPRE3 | | | | | |
| A32 | SynE-mTTR(MD)-TISU- | L15_TISU | TISU- | 4xmiR142 | WPRE3 | sPolyA |
| | miniATP7Bv.s1co3- | | miniATP7B- | | | |
| | miR142-WPRE3 | | s1co3 | | | |
| A33 | SERPINA1cre- | L13_TISU | TISU- | — | WPRE3 | sPolyA |
| | mTTR(MD)-TISU- | | miniATP7Bv.co | | | |
| | miniATP7Bv.co-WPRE3 | | | | | |
| A34 | SERPINA1cre- | L13_TISU | TISU- | — | WPRE3 | sPolyA |
| | mTTR(MD)-TISU- | | miniATP7B- | | | |
| | miniATP7Bv.s1co- | | s1co | | | |
| | WPRE3 | | | | | |
| A35 | SERPINA1cre- | L13_TISU | miniATP7AB | — | WPRE3 | sPolyA |
| | mTTR(MD)-TISU- | | | | | |
| | miniATP7AB-WPRE3 | | | | | |
| A36 | SERPINA1cre- | L13_TISU | TISU- | 4xmiR142 | WPRE3 | sPolyA |
| | mTTR(MD)-TISU- | | miniATP7B- | | | |
| | miniATP7Bv.s1co3- | | s1co3 | | | |
| | mIR142-WPRE3 | | | | | |
| A37 | SERPINA1cre- | L13_TISU | miniATP7AB | — | WPRE250 | sPolyA |
| | mTTR(MD)-TISU- | | | | | |
| | miniATP7AB-WPRE3- | | | | | |
| | pAAV | | | | | |
| A38 | FGBcre-mTTR(MD)- | L10_v2 | miniATP7Bv.co | — | WPRE250 | sPolyA |
| | miniATP7Bv.co- | | | | | |
| | WPRE250 | | | | | |
| A39 | AAT-miniATP7Bv.co- | L1_v2 | miniATP7Bv.co | — | WPRE250 | sPolyA |
| | WPRE250 | | | | | |
| A40 | SynE-mTTR(MD)- | L15 | miniATP7Bv.co | — | WPRE250 | SV40 |
| | miniATP7Bv.co- | | | | | late |
| | WPRE250-SV40polyA | | | | | pA |
| A41 | L57-miniATP7Bv.co- | L57 | miniATP7Bv.co | — | WPRE250 | sPolyA |
| | synPolyA | | | | | |
| A42 | L57-FTL(MD)- | L57 | miniATP7B- | — | WPRE250 | sPolyA |
| | miniATP7B.s1co3- | | s1co3 | | | |
| | 4xmiR142BS-WPRE250 | | | | | |
| A43 | L57-FTL(MD)- | L57 | miniATP7AB | — | WPRE250 | sPolyA |
| | miniATP7AB-WPRE250 | | | | | |
| A44 | L79-miniATP7Bv.co | L79 | miniATP7Bv.co | — | WPRE250 | sPolyA |
| A45 | L89-miniATP7Bv.co | L89 | miniATP7Bv.co | — | WPRE250 | sPolyA |
| A46 | L89-miniATP7Bv.s1co3- | L89 | miniATP7B- | — | WPRE250 | sPolyA |
| | miR142-WPRE250 | | s1co3 | | | |
| A47 | L76-miniATP7Bv.co | L76 | miniATP7Bv.co | — | WPRE250 | sPolyA |
| A48 | L76-miniATP7Bv.s1co3- | L76 | miniATP7B- | — | WPRE250 | sPolyA |
| | miR142-WPRE250 | | s1co3 | | | |
| A49 | L33-miniATP7Bv.co- | L33 | miniATP7Bv.co | — | WPRE250 | sPolyA |
| | WPRE250-synPolyA | | | | | |
| A50 | L33-miniATP7Bv.s1co3- | L33 | miniATP7B- | — | WPRE250 | sPolyA |
| | miR142-WPRE250 | | s1co3 | | | |
| A51 | L16-miniATP7Bv.co | L16 | miniATP7Bv.co | — | WPRE250 | sPolyA |
| A52 | L16-miniATP7Bv.s1co3- | L16 | miniATP7B- | — | WPRE250 | sPolyA |
| | miR142-WPRE250 | | s1co3 | | | |
| A53 | LP1-miniATP7Bv.co- | LP1 | miniATP7Bv.co | — | WPRE250 | sPolyA |
| | WPRE250 | | | | | |
| A54 | LP1-miniATP7Bv.s1co3- | LP1 | miniATP7B- | 4xmiR142 | WPRE250 | sPolyA |
| | miR142-WPRE250 | | s1co3 | | | |
| A55 | LP1v2-miniATP7Bv.co- | LP1v2 | miniATP7Bv.co | — | WPRE250 | sPolyA |
| | WPRE250 | | | | | |
| A56 | LP1v2- | LP1v2 | miniATP7B- | 4xmiR142 | WPRE250 | sPolyA |
| | miniATP7Bv.s1co3- | | s1co3 | | | |
| | miR142-WPRE250 | | | | | |
| A57 | LP1v3-miniATP7Bv.co- | LP1v3 | miniATP7Bv.co | — | WPRE250 | sPolyA |
| | WPRE250 | | | | | |
| A58 | LP1v3- | LP1v3 | miniATP7B- | 4xmiR142 | WPRE250 | sPolyA |
| | miniATP7Bv.s1co3- | | s1co3 | | | |
| | miR142-WPRE250 | | | | | |
| A59 | LP1v4-miniATP7Bv.co- | LP1v4 | miniATP7Bv.co | — | WPRE250 | sPolyA |
| | WPRE250 | | | | | |
| A60 | LP1v4- | LP1v4 | miniATP7B- | 4xmiR142 | WPRE250 | sPolyA |
| | miniATP7Bv.s1co3- | | s1co3 | | | |
| | miR142-WPRE250 | | | | | |
| A61 | LP1v5-miniATP7Bv.co- | LP1v5 | miniATP7Bv.co | — | WPRE250 | sPolyA |
| | WPRE250 | | | | | |
| A62 | LP1v5- | LP1v5 | miniATP7B- | 4xmiR142 | WPRE250 | sPolyA |
| | miniATP7Bv.s1co3- | | s1co3 | | | |
| | miR142-WPRE250 | | | | | |

TABLE 8
Promoter sequences. SN: = SEQ ID NO.

| | | Alternative | |
|---|---|---|---|
| SN: | Name | name | Sequence |
| 1 | L1 | AAT | CACCCCCTCCACCTTGGACACAGGACGCTGTGGTT |
| | | | TCTGAGCCAGGTACAATGACTCCTTTCGGTAAGTG |
| | | | CAGTGGAAGCTGTACACTGCCCAGGCAAAGCGTCC |
| | | | GGGCAGCGTAGGCGGGCGACTCAGATCCCAGCCAG |
| | | | TGGACTTAGCCCCTGTTTGCTCCTCCGATAACTGG |
| | | | GGTGACCTTGGTTAATATTCACCAGCAGCCTCCCC |
| | | | CGTTGCCCCTCTGGATCCACTGCTTAAATACGGAC |
| | | | GAGGACAGGGCCCTGTCTCCTCAGCTTCAGGCACC |
| | | | ACCACTGACCTGGGACAGTGAATCGGCGGCCGCCA |
| | | | CC |
| 2 | LP1 | | CCCTAAAATGGGCAAACATTGCAAGCAGCAAACAG |
| | | | CAAACACACAGCCCTCCCTGCCTGCTGACCTTGGA |
| | | | GCTGGGGCAGAGGTCAGAGACCTCTCTGGGCCCAT |
| | | | GCCACCTCCAACATCCACTCGACCCCTTGGAATTT |
| | | | CGGTGGAGAGGAGCAGAGGTTGTCCTGGCGTGGTT |
| | | | TAGGTAGTGTGAGAGGGGAATGACTCCTTTCGGTA |
| | | | AGTGCAGTGGAAGCTGTACACTGCCCAGGCAAAGC |
| | | | GTCCGGGCAGCGTAGGCGGGCGACTCAGATCCCAG |
| | | | CCAGTGGACTTAGCCCCTGTTTGCTCCTCCGATAA |
| | | | CTGGGGTGACCTTGGTTAATATTCACCAGCAGCCT |
| | | | CCCCCGTTGCCCCTCTGGATCCACTGCTTAAATAC |
| | | | GGACGAGGACAGGGCCCTGTCTCCTCAGCTTCAGG |
| | | | CACCACCACTGACCTGGGACAGTGAATCGGCGGCC |
| | | | GCCACC |
| 3 | L68 | HLP | TGTTTGCTGCTTGCAATGTTTGCCCATTTTAGGGT |
| | | | GGACACAGGACGCTGTGGTTTCTGAGCCAGGGGGC |
| | | | GACTCAGATCCCAGCCAGTGGACTTAGCCCCTGTT |
| | | | TGCTCCTCCGATAACTGGGGTGACCTTGGTTAATA |
| | | | TTCACCAGCAGCCTCCCCCGTTGCCCCTCTGGATC |
| | | | CACTGCTTAAATACGGACGAGGACAGGGCCCTGTC |
| | | | TCCTCAGCTTCAGGCACCACCACTGACCTGGGACA |
| | | | GTGAATCGCGGCCGCCACC |
| 4 | L1 v2 | | CACCCCCTCCACCTTGGACACAGGACGCTGTGGTT |
| | | | TCTGAGCCAGGTACAATGACTCCTTTCGGTAAGTG |
| | | | CAGTGGAAGCTGTACACTGCCCAGGCAAAGCGTCC |
| | | | GGGCAGCGTAGGCGGGCGACTCAGATCCCAGCCAG |
| | | | TGGACTTAGCCCCTGTTTGCTCCTCCGATAACTGG |
| | | | GGTGACCTTGGTTAATATTCACCAGCAGCCTCCCC |
| | | | CGTTGCCCCTCTGGATCCACTGCTTAAATACGGAC |
| | | | GAGGACAGGGCCCTGTCTCCTCAGCTTCAGGCACC |
| | | | ACCACTGACCTGGGACAGTGAAGCGGCCGCCACC |
| 5 | L10 | | GAATTTTAATAAATACCTCTCTGTCCTTCTCCTAG |
| | | | CTTCACCCTTTGTTTGTTGAAAGGAGATACTTTAG |
| | | | TGCAGTATTGCAACAGCTTATGTAGACATGCAACC |
| | | | TTGAGGTCACAGACCTATGAGGGATTGAGACAAAG |
| | | | GCAAATTTAGCAAATATTTTCCAGAGTACTCTGCT |
| | | | GCAATAACGCCGCGTTCGCCCCGCCCCCGACGGTA |
| | | | TCGATAAGCTTAGGAGCTTGGGCTGCAGGTCGAGG |
| | | | GCACTGGGAGGATGTTGAGTAAGATGGAAAACTAC |
| | | | TGATGACCCTTGCAGAGACAGAGTATTAGGACATG |
| | | | TTTGAACAGGGGCCGGGCGATCAGCAGGTAGCTCT |
| | | | AGAGGATCCCCGTCTGTCTGCACATTTCGTAGAGC |
| | | | GAGTGTTCCGATACTCTAATCTCCCTAGGCAAGGT |
| | | | TCATATTTGTGTAGGTTACTTATTCTCCTTTTGTT |
| | | | GACTAAGTCAATAATCAGAATCAGCAGGTTTGGAG |
| | | | TCAGCTTGGCAGGGATCAGCAGCCTGGGTTGGAAG |
| | | | GAGGGGGTATAAAAGCCCCTTCACCAGGAGAAGCC |
| | | | GTCACACAGATCCACAAGCTCGAGCCGAGTGGTCG |
| | | | TGCCTCCATAGAAGCGGCCGCCACC |
| 6 | L10 | FGBcre1- | GAATTTTAATAAATACCTCTCTGTCCTTCTCCTAG |
| | | mTTR | CTTCACCCTTTGTTTGTTGAAAGGAGATACTTTAG |
| | | (MPE_DPE) | TGCAGTATTGCAACAGCTTATGTAGACATGCAACC |
| | | | TTGAGGTCACAGACCTATGAGGGATTGAGACAAAG |
| | | | GCAAATTTAGCAAATATTTTCCAGAGTACTCTGCT |
| | | | GCAATAACGCCGCGTTCGCCCCGCCCCCGACGGTA |
| | | | TCGATAAGCTTAGGAGCTTGGGCTGCAGGTCGAGG |
| | | | GCACTGGGAGGATGTTGAGTAAGATGGAAAACTAC |
| | | | TGATGACCCTTGCAGAGACAGAGTATTAGGACATG |
| | | | TTTGAACAGGGGCCGGGCGATCAGCAGGTAGCTCT |
| | | | AGAGGATCCCCGTCTGTCTGCACATTTCGTAGAGC |
| | | | GAGTGTTCCGATACTCTAATCTCCCTAGGCAAGGT |
| | | | TCATATTTGTGTAGGTTACTTATTCTCCTTTTGTT |
| | | | GACTAAGTCAATAATCAGAATCAGCAGGTTTGGAG |
| | | | TCAGCTTGGCAGGGATCAGCAGCCTGGGTTGGAAG |
| | | | GAGGGGGTATAAAAGCCCCTTCACCAGGAGAAGCC |
| | | | GTCACACAGATCCACAAGCTCGAGCCGAGTGGTCG |
| | | | TGCCTCCATAGAAGCGGCCGCCACC |
| 7 | L10_v2 | | GAATTTTAATAAATACCTCTCTGTCCTTCTCCTAG |
| | | | CTTCACCCTTTGTTTGTTGAAAGGAGATACTTTAG |
| | | | TGCAGTATTGCAACAGCTTATGTAGACATGCAACC |
| | | | TTGAGGTCACAGACCTATGAGGGATTGAGACAAAG |
| | | | GCAAATTTAGCAAATATTTTCCAGAGTACTCTGCT |
| | | | GCAATAACCGCGTTCGCCCCGCCCCGGACGGTATC |
| | | | GATAAGCTTAGGAGCTTGGGCTGCAGGTCGAGGGC |
| | | | ACTGGGAGGATGTTGAGTAAGATGGAAAACTACTG |
| | | | ATGACCCTTGCAGAGACAGAGTATTAGGACATGTT |
| | | | TGAACAGGGGCCGGGCGATCAGCAGGTAGCTCTAG |
| | | | AGGATCCCCGTCTGTCTGCACATTTCGTAGAGCGA |
| | | | GTGTTCCGATACTCTAATCTCCCTAGGCAAGGTTC |
| | | | ATATTTGTGTAGGTTACTTATTCTCCTTTTGTTGA |
| | | | CTAAGTCAATAATCAGAATCAGCAGGTTTGGAGTC |
| | | | AGCTTGGCAGGGATCAGCAGCCTGGGTTGGAAGGA |
| | | | GGGGGTATAAAAGCCCCTTCACCAGGAGAAGCCGT |
| | | | CACACAGATCCACAAGCTCGAGCCGAGTGGTCGTG |
| | | | CCTCCATAGAAGCGGCCGCCACC |
| 8 | L13 | | GAGTTGCTGGTGCTTCCCCAGGCTGGAGATTGAGT |
| | | | TAATATTAACAGGCCCAAGGCGATGTGGGCTTGTG |
| | | | CAATCATAGGCCCGGCCTCGCCAGGTCACCTGAGG |
| | | | AGTTAATGAATACATATCTCCTGCCGCGTTCGCCC |
| | | | CGCCCCCGACGGTATCGATAAGCTTAGGAGCTTGG |
| | | | GCTGCAGGTCGAGGGCACTGGGAGGATGTTGAGTA |
| | | | AGATGGAAAACTACTGATGACCCTTGCAGAGACAG |
| | | | AGTATTAGGACATGTTTGAACAGGGGCCGGGCGAT |
| | | | CAGCAGGTAGCTCTAGAGGATCCCCGTCTGTCTGC |
| | | | ACATTTCGTAGAGCGAGTGTTCCGATACTCTAATC |
| | | | TCCCTAGGCAAGGTTCATATTTGTGTAGGTTACTT |
| | | | ATTCTCCTTTTGTTGACTAAGTCAATAATCAGAAT |
| | | | CAGCAGGTTTGGAGTCAGCTTGGCAGGGATCAGCA |
| | | | GCCTGGGTTGGAAGGAGGGGGTATAAAAGCCCCTT |
| | | | CACCAGGAGAAGCCGTCACACAGATCCACAAGCTC |
| | | | GAGCCGAGTGGTCGTGCCTCCATAGAAGCGGCCGC |
| | | | CACC |
| 9 | L13_ | | GAGTTGCTGGTGCTTCCCCAGGCTGGAGATTGAGT |
| | TISU | | TAATATTAACAGGCCCAAGGCGATGTGGGCTTGTG |
| | | | CAATCATAGGCCCGGCCTCGCCAGGTCACCTGAGG |
| | | | AGTTAATGAATACATATCTCCTGCCGCGTTCGCCC |
| | | | CGCCCCCGACGGTATCGATAAGCTTAGGAGCTTGG |
| | | | GCTGCAGGTCGAGGGCACTGGGAGGATGTTGAGTA |
| | | | AGATGGAAAACTACTGATGACCCTTGCAGAGACAG |
| | | | AGTATTAGGACATGTTTGAACAGGGGCCGGGCGAT |
| | | | CAGCAGGTAGCTCTAGAGGATCCCCGTCTGTCTGC |
| | | | ACATTTCGTAGAGCGAGTGTTCCGATACTCTAATC |
| | | | TCCCTAGGCAAGGTTCATATTTGTGTAGGTTACTT |
| | | | ATTCTCCTTTTGTTGACTAAGTCAATAATCAGAAT |
| | | | CAGCAGGTTTGGAGTCAGCTTGGCAGGGATCAGCA |
| | | | GCCTGGGTTGGAAGGAGGGGGTATAAAAGCCCCTT |
| | | | CACCAGGAGAAGCCGTCACACAGATCCACAAGCTC |
| | | | GAGCCGAGTGGTCGTGCCTCCATAGAAGCGGCCGC |
| | | | AAG |
| 10 | L13_v2 | | GAGTTGCTGGTGCTTCCCCAGGCTGGAGATTGAGT |
| | | | TAATATTAACAGGCCCAAGGCGATGTGGGCTTGTG |
| | | | CAATCATAGGCCCGGCCTCGCCAGGTCACCTGAGG |
| | | | AGTTAATGAATACATATCTCCTGCCGCGTTCGCCC |
| | | | CGCCCCCGACGGTATCGATAAGCTTAGGAGCTTGG |
| | | | GCTGCAGGTCGAGGGCACTGGGAGGATGTTGAGTA |
| | | | AGATGGAAAACTACTGATGACCCTTGCAGAGACAG |
| | | | AGTATTAGGACATGTTTGAACAGGGGCCGGGCGAT |
| | | | CAGCAGGTAGCTCTAGAGGATCCCCGTCTGTCTGC |
| | | | ACATTTCGTAGAGCGAGTGTTCCGATACTCTAATC |
| | | | TCCCTAGGCAAGGTTCATATTTGTGTAGGTTACTT |
| | | | ATTCTCCTTTTGTTGACTAAGTCAATAATCAGAAT |
| | | | CAGCAGGTTTGGAGTCAGCTTGGCAGGGATCAGCA |
| | | | GCCTGGGTTGGAAGGAGGGGGTATAAAAGCCCCTT |
| | | | CACCAGGAGAAGCCGTCACACAGATCCACAAGCTC |
| | | | GAGCCGAGTGGTCGTGCCTCCATAGAAGCGGCCGC |
| | | | AACTCCTAAAAAACCGCCACC |
| 11 | L15 | | GAGTTAATAATTACCAGCGCGGGCCAAATAAATAA |
| | | | TCCGCGAGGGGCAGCTGGACTTTGGACTCCGCGCG |
| | | | CTGGTAATTATTAACCTCGCGAATATTGATTCGAG |
| | | | GCCGCGATTGCCGCAATCGCGAGGGGCAGGTGACC |
| | | | TTTGCCCAGCGCGCGTTCGCCCCGCCCCGGACGGT |
| | | | ATCGATAAGCTTAGGAGCTTGGGCTGCAGGTCGAG |
| | | | GGCACTGGGAGGATGTTGAGTAAGATGGAAAACTA |
| | | | CTGATGACCCTTGCAGAGACAGAGTATTAGGACAT |
| | | | GTTTGAACAGGGGCCGGGCGATCAGCAGGTAGCTC |
| | | | TAGAGGATCCCCGTCTGTCTGCACATTTCGTAGAG |
| | | | CGAGTGTTCCGATACTCTAATCTCCCTAGGCAAGG |
| | | | TTCATATTTGTGTAGGTTACTTATTCTCCTTTTGT |
| | | | TGACTAAGTCAATAATCAGAATCAGCAGGTTTGGA |
| | | | GTCAGCTTGGCAGGGATCAGCAGCCTGGGTTGGAA |
| | | | GGAGGGGGTATAAAAGCCCCTTCACCAGGAGAAGC |
| | | | CGTCACACAGATCCACAAGCTCGAGCCGAGTGGTC |
| | | | GTGCCTCCATAGAAGCGGCCGCCACC |
| 12 | L15_21 | | GAGTTAATAATTACCAGCGCGGGCCAAATAAATAA |
| | | | TCCGCGAGGGGCAGCTGGACTTTGGACTCCGCGCG |
| | | | CTGGTAATTATTAACCTCGCGAATATTGATTCGAG |
| | | | GCCGCGATTGCCGCAATCGCGAGGGGCAGGTGACC |
| | | | TTTGCCCAGCGCGCGTTCGCCCCGCCCCGGACGGT |
| | | | ATCGATAAGCTTAGGAGCTTGGGCTGCAGGTCGAG |
| | | | GGCACTGGGAGGATGTTGAGTAAGATGGAAAACTA |
| | | | CTGATGACCCTTGCAGAGACAGAGTATTAGGACAT |
| | | | GTTTGAACAGGGGCCGGGCGATCAGCAGGTAGCTC |
| | | | TAGAGGATCCCCGTCTGTCTGCACATTTCGTAGAG |
| | | | CGAGTGTTCCGATACTCTAATCTCCCTAGGCAAGG |
| | | | TTCATATTTGTGTAGGTTACTTATTCTCCTTTTGT |
| | | | TGACTAAGTCAATAATCAGAATCAGCAGGTTTGGA |
| | | | GTCAGCTTGGCAGGGATCAGCAGCCTGGGTTGGAA |
| | | | GGAGGGGGTATAAAAGCCCCTTCACCAGGAGAAGC |
| | | | CGTCACACAGATCCACAAGCTCGAGCCGAGTGGTC |
| | | | GTGCCTCCATAGAAGCGGCCGCAACTCCTAAAAAA |
| | | | CCGCCACC |
| 13 | L15_21 | | GAGTTAATAATTACCAGCGCGGGCCAAATAAATAA |
| | _TISU | | TCCGCGAGGGGCAGCTGGACTTTGGACTCCGCGCG |
| | | | CTGGTAATTATTAACCTCGCGAATATTGATTCGAG |
| | | | GCCGCGATTGCCGCAATCGCGAGGGGCAGGTGACC |
| | | | TTTGCCCAGCGCGCGTTCGCCCCGCCCCGGACGGT |
| | | | ATCGATAAGCTTAGGAGCTTGGGCTGCAGGTCGAG |
| | | | GGCACTGGGAGGATGTTGAGTAAGATGGAAAACTA |
| | | | CTGATGACCCTTGCAGAGACAGAGTATTAGGACAT |
| | | | GTTTGAACAGGGGCCGGGCGATCAGCAGGTAGCTC |
| | | | TAGAGGATCCCCGTCTGTCTGCACATTTCGTAGAG |
| | | | CGAGTGTTCCGATACTCTAATCTCCCTAGGCAAGG |
| | | | TTCATATTTGTGTAGGTTACTTATTCTCCTTTTGT |
| | | | TGACTAAGTCAATAATCAGAATCAGCAGGTTTGGA |
| | | | GTCAGCTTGGCAGGGATCAGCAGCCTGGGTTGGAA |
| | | | GGAGGGGGTATAAAAGCCCCTTCACCAGGAGAAGC |
| | | | CGTCACACAGATCCACAAGCTCGAGCCGAGTGGTC |
| | | | GTGCCTCCATAGAAGCGGCCGCAACTCCTAAAAAA |
| | | | CCGCCAAGATGGCGGC |
| 14 | L15_ | | GAGTTAATAATTACCAGCGCGGGCCAAATAAATAA |
| | TISU | | TCCGCGAGGGGCAGCTGGACTTTGGACTCCGCGCG |
| | | | CTGGTAATTATTAACCTCGCGAATATTGATTCGAG |
| | | | GCCGCGATTGCCGCAATCGCGAGGGGCAGGTGACC |
| | | | TTTGCCCAGCGCGCGTTCGCCCCGCCCCGGACGGT |
| | | | ATCGATAAGCTTAGGAGCTTGGGCTGCAGGTCGAG |
| | | | GGCACTGGGAGGATGTTGAGTAAGATGGAAAACTA |
| | | | CTGATGACCCTTGCAGAGACAGAGTATTAGGACAT |
| | | | GTTTGAACAGGGGCCGGGCGATCAGCAGGTAGCTC |
| | | | TAGAGGATCCCCGTCTGTCTGCACATTTCGTAGAG |
| | | | CGAGTGTTCCGATACTCTAATCTCCCTAGGCAAGG |
| | | | TTCATATTTGTGTAGGTTACTTATTCTCCTTTTGT |
| | | | TGACTAAGTCAATAATCAGAATCAGCAGGTTTGGA |
| | | | GTCAGCTTGGCAGGGATCAGCAGCCTGGGTTGGAA |
| | | | GGAGGGGGTATAAAAGCCCCTTCACCAGGAGAAGC |
| | | | CGTCACACAGATCCACAAGCTCGAGCCGAGTGGTC |
| | | | GTGCCTCCATAGAAGCGGCCGCAAG |
| 15 | L16 | | GAGTTAATAATTACCAGCGCGGGCCAAATAAATAA |
| | | | TCCGCGAGGGGCAGCTGGACTTTGGACTCCGCGCG |
| | | | CTGGTAATTATTAACCTCGCGAATATTGATTCGAG |
| | | | GCCGCGATTGCCGCAATCGCGAGGGGCAGGTGACC |
| | | | TTTGCCCAGCGCGCGTTCGCCCCGCCCCGGACCAC |
| | | | CCCCTCCACCTTGGACACAGGACGCTGTGGTTTCT |
| | | | GAGCCAGGTACAATGACTCCTTTCGGTAAGTGCAG |
| | | | TGGAAGCTGTACACTGCCCAGGCAAAGCGTCCGGG |
| | | | CAGCGTAGGCGGGCGACTCAGATCCCAGCCAGTGG |
| | | | ACTTAGCCCCTGTTTGCTCCTCCGATAACTGGGGT |
| | | | GACCTTGGTTAATATTCACCAGCAGCCTCCCCCGT |
| | | | TGCCCCTCTGGATCCACTGCTTAAATACGGACGAG |
| | | | GACAGGGCCCTGTCTCCTCAGCTTCAGGCACCACC |
| | | | ACTGACCTGGGACAGTGAATCGAAGAGGTAAGGGT |
| | | | TTAAGGGATGGTTGGTTGGTGGGGTATTAATGTTT |
| | | | AATTACCTGGAGCACCTGCCTGAAATCACTTTTTT |
| | | | TCAGGTTGGGCGGCCGCCACC |
| 16 | L17_ | SynEnh | GAGTTAATAATTACCAGCGCGGGCCAAATAAATAA |
| | mTTR | _v2- | TCCGCGAGGGGCAGCTGGACTTTGGACTCCGCGCG |
| | | | CTGGTAATTATTAACCTCGCGAATATTGATTCGAG |
| | | | GCCGCGATTGCCGCAATCGCGAGGGGCAGGTGACC |
| | | | TTTGCCCAGCGCGCGTTCGCCCCGCCCCGGACGGT |
| | | | ATCGATAAGCTTAGGAGCTTGGGCTGCAGGTCGAG |
| | | | GGCACTGGGAGGATGTTGAGTAAGATGGAAAACTA |
| | | | CTGATGACCCTTGCAGAGACAGAGTATTAGGACAT |
| | | | GTTTGAACAGGGGCCGGGCGATCAGCAGGTAGCTC |
| | | | TAGAGGATCCCCGTCTGTCTGCACATTTCGTAGAG |
| | | | CGAGTGTTCCGATACTCTAATCTCCCTAGGCAAGG |
| | | | TTCATATTTGTGTAGGTTACTTATTCTCCTTTTGT |
| | | | TGACTAAGTCAATAATCAGAATCAGCAGGTTTGGA |
| | | | GTCAGCTTGGCAGGGATCAGCAGCCTGGGTTGGAA |
| | | | GGAGGGGGTATAAAAGCCCCTTCACCAGGAGAAGC |
| | | | CGTCACACAGATCCACAAGCTCCTGACAGGGCGGC |
| | | | CGCCACC |
| 17 | L33 | | GAGTTAATAATTACCAGCGCGGGCCAAATAAATAA |
| | | | TCCGCGAGGGGCAGCTGGACTTTGGACTCCGCGCG |
| | | | CTGGTAATTATTAACCTCGCGAATATTGATTCGAG |
| | | | GCCGCGATTGCCGCAATCGCGAGGGGCAGGTGACC |
| | | | TTTGCCCAGCGGGTACCCGGGGGCGCGCCAGTCAC |
| | | | CCCCTCCACCTTGGACACAGGACGCTGTGGTTTCT |
| | | | GAGCCAGGTACAATGACTCCTTTCGGTAAGTGCAG |
| | | | TGGAAGCTGTACACTGCCCAGGCAAAGCGTCCGGG |
| | | | CAGCGTAGGCGGGCGACTCAGATCCCAGCCAGTGG |
| | | | ACTTAGCCCCTGTTTGCTCCTCCGATAACTGGGGT |
| | | | GACCTTGGTTAATATTCACCAGCAGCCTCCCCCGT |
| | | | TGCCCCTCTGGATCCACTGCTTAAATACGGACGAG |
| | | | GACAGGGCCCTGTCTCCTCAGCTTCAGGCACCACC |
| | | | ACTGACCTGGGACAGTGAATCGGCGGCCGCCACC |
| 18 | L45 | FTL | TCCCATAAAACTCTAGCCTCCCAGAGGGACCCCAG |
| | | | CCCCCACCCTCCCGCCCACGAACCCCTGCATTTCC |
| | | | AGAATCAGCCCCAGGGCCCCAACCCCCCCAAGCCC |
| | | | CCATTTCACAACACGCTGGCGCTACAGGCGCGTGA |
| | | | CTTCCCCTTGCTTTGGGGCGGGGGGCTGAGACTCC |
| | | | TATGTGCTCCGGATTGGTCAGGCACGGCCTTCGGC |
| | | | CCCGCCTCCTGCCACCGCAGATTGGCCGCTAGCCC |
| | | | TCCCCGAGCGCCCTGCCTCCGAGGGCCGGCGCACC |
| | | | ATAAAAGAAGCCGCCCTAGCCACGTCCCCTCGCAG |
| | | | TTCGGCGGTCCCGCGGGTCTGTCTCTTGCTTCAAC |
| | | | AGTGTTTGGACGGAACAGATCCGGGGACTCTCTTC |
| | | | CAGCCTCCGACCGCCCTCCGATTTCCTCTCCGCTT |
| | | | GCAACCTCCGGGACCATCTTCTCGGCCATCTCCTG |
| | | | CTTCTGGGACCTGCCAGCACCGTTTTTGTGGTTAG |
| | | | CTCCTTCTTGCCAACCAACCGGATCCGCCACC |
| 19 | L57 | | TCCCATAAAACTCTAGCCTCCCAGAGGGACCCCAG |
| | | | CCCCCACCCTCCCGCCCACGAACCCCTGCATTTCC |
| | | | AGAATCAGCCCCAGGGCCCCAACCCCCCCAAGCCC |
| | | | CCATTTCACAACACGCTGGCGCTACAGGCGCGTGA |
| | | | CTTCCCCTTGCTTTGGGGCGGGGGGCTGAGACTCC |
| | | | TATGTGCTCCGGATTGGTCAGGCACGGCCTTCGGC |
| | | | CCCGCCTCCTGCCACCGCAGATTGGCCGCTAGCCC |
| | | | TCCCCGAGCGCCCTGCCTCCGAGGGCCGGCGCACT |
| | | | ATAAAAGCCCCTTCACCAGGAGAAGCCGTCACACA |
| | | | GATCCACAAGCTCGAGCCGAGTGGTCGTGGGTCTG |
| | | | TCTCTTGCTTCAACAGTGTTTGGACGGAACAGATC |
| | | | CGGGGACTCTCTTCCAGCCTCCGACCGCCCTCCGA |
| | | | TTTCCTCTCCGCTTGCAACCTCCGGGACCATCTTC |
| | | | TCGGCCATCTCCTGCTTCTGGGACCTGCCAGCACC |
| | | | GTTTTTGTGGTTAGCTCCTTCTTGCCAACCAACCG |
| | | | GATCCGCCACC |
| 20 | L6 | ALBcre- | AGCCAATGAAATACAAAGATGAGTCTAGTTAATAA |
| | | mTTR | TCTACAATTATTGGTTAAAGAAGTATATTAGTGCT |
| | | | AATTTCCCTCCGTTTGTCCTAGCTTTTCTCGCCGC |
| | | | GTTCGCCCCGCCCCCGACGGTATCGATAAGCTTAG |
| | | | GAGCTTGGGCTGCAGGTCGAGGGCACTGGGAGGAT |
| | | | GTTGAGTAAGATGGAAAACTACTGATGACCCTTGC |
| | | | AGAGACAGAGTATTAGGACATGTTTGAACAGGGGC |
| | | | CGGGCGATCAGCAGGTAGCTCTAGAGGATCCCCGT |
| | | | CTGTCTGCACATTTCGTAGAGCGAGTGTTCCGATA |
| | | | CTCTAATCTCCCTAGGCAAGGTTCATATTTGTGTA |
| | | | GGTTACTTATTCTCCTTTTGTTGACTAAGTCAATA |
| | | | ATCAGAATCAGCAGGTTTGGAGTCAGCTTGGCAGG |
| | | | GATCAGCAGCCTGGGTTGGAAGGAGGGGGTATAAA |
| | | | AGCCCCTTCACCAGGAGAAGCCGTCACACAGATCC |
| | | | ACAAGCTCGAGCCGAGTGGTCGTGCCTCCATAGAA |
| | | | GCGGCCGCCACC |
| 21 | L61 | FTL6 | GAGTTGCTGGTGCTTCCCCAGGCTGGAGATTGAGT |
| | | | TAATATTAACAGGCCCAAGGCGATGTGGGCTTGTG |
| | | | CAATCATAGGCCCGGCCTCGCCAGGTCACCTGAGG |
| | | | AGTTAATGAATACATATCTCCTGAGTTAATAATTA |
| | | | CCAGCGCGGGCCAAATAAATAATCCGCGAGGGGCA |
| | | | GCTGGACTTTGGACTCCGCGCGCTGGTAATTATTA |
| | | | ACCTCGCGAATATTGATTCGAGGCCGCGATTGCCG |
| | | | CAATCGCGAGGGGCAGGTGACCTTTGCCCAGCGCG |
| | | | CGTTCGCCCCGCCCCGGACGGTATCGATAAGCTTA |
| | | | GGAGCTTGGGCTGCAGGTCGAGGTCCCATAAAACT |
| | | | CTAGCCTCCCAGAGGGACCCCAGCCCCCACCCTCC |
| | | | CGCCCACGAACCCCTGCATTTCCAGAATCAGCCCC |
| | | | AGGGCCCCAACCCCCCCAAGCCCCCATTTCACAAC |
| | | | ACGCTGGCGCTACAGGCGCGTGACTTCCCCTTGCT |
| | | | TTGGGGCGGGGGGCTGAGACTCCTATGTGCTCCGG |
| | | | ATTGGTCAGGCACGGCCTTCGGCCCCGCCTCCTGC |
| | | | CACCGCAGATTGGCCGCTAGCCCTCCCCGAGCGCC |
| | | | CTGCCTCCGAGGGCCGGCGCACTATAAAAGCCCCT |
| | | | TCACCAGGAGAAGCCGTCACACAGATCCACAAGCT |
| | | | CGAGCCGAGTGGTCGTGGGTCTGTCTCTTGCTTCA |
| | | | ACAGTGTTTGGACGGAACAGATCCGGGGACTCTCT |
| | | | TCCACCAACCAACCGGATCCGCCACC |
| 22 | L73 | FTL10 | TCCCATAAAACTCTAGCCTCCCAGAGGGACCCCAG |
| | | | CCCCCACCCTCCCGCCCACGAACCCCTGCATTTCC |
| | | | AGAATCAGCCCCAGGGCCCCAACCCCCCCAAGCCC |
| | | | CCATTTCACAACACGCTGGCGCTACAGGCGCGTGA |
| | | | CTTCCCCTTGCTTTGGGGCGGGGGGCTGAGACTCC |
| | | | TATGTGCTCCGGATTGGTCAGGCACGGCCTTCGGC |
| | | | CCCGCCTCCTGCCACCGCAGATTGGCCGCTAGCCC |
| | | | TCCCCGAGCGCCCTGCCTCCGAGGGCCGGCGCACT |
| | | | ATAAAAGCCCCTTCACCAGGAGAAGCCGTCACACA |
| | | | GATCCACAAGCTCGAGCCGAGTGGTCGTGGGTCTG |
| | | | TCTCTTGCTTCAACAGTGTTTGGACGGAACAGATC |
| | | | CGGGGACTCTCTTCCAGCCTCCGACCGCCCTCCGA |
| | | | TTTCCTCTCCGCTTGCAACCTCCGGGACCATCTTC |
| | | | TCGGCCATCTCCTGCTTCTGGGACCTGCCAGCACC |
| | | | GTTTTTGTGGTTAGCTCCTGGGCAGGTTGGTATCA |
| | | | AGGTTACAAGACAGGTTTAAGGAGACCAATAGAAA |
| | | | CTGGGCATGTGGAGACAGGCACTGGGAGGATGTTG |
| | | | AGTAAGATGGAAAACTACTGATGACCCTTGCAGAG |
| | | | ACAGAGTATTAGGACATGTTTGAACAGGGGCCGGG |
| | | | CGATCAGCAGGTAGCTCTAGAGGATCCCCGTCTGT |
| | | | CTGCACATTTCGTAGAGCGAGTGTTCCGATACTCT |
| | | | AATCTCCCTAGGCAAGGTTCATATTTGTGTAGGTT |
| | | | ACTTATTCTCCTTTTGTTGACTAAGTCAATAATCA |
| | | | GAATCAGCAGGTTTGGAGTCAGCTTGGCAGGGATC |
| | | | AGCAGCCTGGGTTGGAAGGAGGGGGTATAAAAGCC |
| | | | CCTTCACCAGGAGAAGCCGTCACACAGATCCACAA |
| | | | GCTCGAGCCGAGTGGTCGTAGAAGACTCTTGGGTT |
| | | | TCTGATAGGCACTGACTCTCTCTGCCTATTGGTCT |
| | | | ATTTTCCCACCCTTAGGCTGCTTCTTGCCAACCAA |
| | | | CC |
| 23 | L74 | FTL11 | GAGTTGCTGGTGCTTCCCCAGGCTGGAGATTGAGT |
| | | | TAATATTAACAGGCCCAAGGCGATGTGGGCTTGTG |
| | | | CAATCATAGGCCCGGCCTCGCCAGGTCACCTGAGG |
| | | | AGTTAATGAATACATATCTCCTGAGTTAATAATTA |
| | | | CCAGCGCGGGCCAAATAAATAATCCGCGAGGGGCA |
| | | | GCTGGACTTTGGACTCCGCGCGCTGGTAATTATTA |
| | | | ACCTCGCGAATATTGATTCGAGGCCGCGATTGCCG |
| | | | CAATCGCGAGGGGCAGGTGACCTTTGCCCAGCGCG |
| | | | CGTTCGCCCCGCCCCGGACGGTATCGATAAGCTTA |
| | | | GGAGCTTGGGCTGCAGGTCGAGGTCCCATAAAACT |
| | | | CTAGCCTCCCAGAGGGACCCCAGCCCCCACCCTCC |
| | | | CGCCCACGAACCCCTGCATTTCCAGAATCAGCCCC |
| | | | AGGGCCCCAACCCCCCCAAGCCCCCATTTCACAAC |
| | | | ACGCTGGCGCTACAGGCGCGTGACTTCCCCTTGCT |
| | | | TTGGGGCGGGGGGCTGAGACTCCTATGTGCTCCGG |
| | | | ATTGGTCAGGCACGGCCTTCGGCCCCGCCTCCTGC |
| | | | CACCGCAGATTGGCCGCTAGCCCTCCCCGAGCGCC |
| | | | CTGCCTCCGAGGGCCGGCGCACTATAAAAGCCCCT |
| | | | TCACCAGGAGAAGCCGTCACACAGATCCACAAGCT |
| | | | CGAGCCGAGTGGTCGTGGGTCTGTCTCTTGCTTCA |
| | | | ACAGTGTTTGGACGGAACAGATCCGGGGACTCTCT |
| | | | TCCACCTCCGACCGCCCTCCGATTTCCTCTCCGCT |
| | | | TGCAACCTCCGGGACCATCTTCTCGGCCATCTCCT |
| | | | GCTTCTGGGACCTGCCAGCACCGTTTTTGTGGTTA |
| | | | GCTCCTCCAACCAACCGGATCCGCCACC |
| 24 | L75 | HCB- | GTTAATCATTAAGTCGTTAATTTTTGTGGCCCTTG |
| | | MVMi | CGATGTTTGCTCTGGTTAATAATCTCAGGACAAAC |
| | | | AGAGGTTAATAATTTTCCAGATCTCTCTGAGCAAT |
| | | | AGTATAAAAGGCCAGCAGCAGCCTGACCACATCTC |
| | | | ATCCTTGAGCCAAGAGGTAAGGGTTTAAGGGATGG |
| | | | TTGGTTGGTGGGGTATTAATGTTTAATTACCTGGA |
| | | | GCACCTGCCTGAAATCACTTTTTTTCAGGTTGGGG |
| | | | ATCCGCCACC |
| 25 | L76 | | GTTAATCATTAAGTCGTTAATTTTTGTGGCCCTTG |
| | | | CGATGTTTGCTCTGGTTAATAATCTCAGGACAAAC |
| | | | AGAGGTTAATAATTTTCCAGATCTCTCTGAGCAAT |
| | | | AGTATAAAAGGCCAGCAGCAGCCTGACCACATCTC |
| | | | ATCCTTGATCCACAAGCTCGAGCCGAGTGGTCGTG |
| | | | AGCCAAGAGGTAAGGGTTTAAGGGATGGTTGGTTG |
| | | | GTGGGGTATTAATGTTTAATTACCTGGAGCACCTG |
| | | | CCTGAAATCACTTTTTTTCAGGTTGGGGATCCGCC |
| | | | ACC |
| 26 | L79 | | GAGTTGCTGGTGCTTCCCCAGGCTGGAGATTGAGT |
| | | | TAATATTAACAGGCCCAAGGCGATGTGGGCTTGTG |
| | | | CAATCATAGGCCCGGCCTCGCCAGGTCACCTGAGG |
| | | | AGTTAATGAATACATATCTCCTGAGTTAATAATTA |
| | | | CCAGCGCGGGCCAAATAAATAATCCGCGAGGGGCA |
| | | | GCTGGACTTTGGACTCCGCGCGCTGGTAATTATTA |
| | | | ACCTCGCGAATATTGATTCGAGGCCGCGATTGCCG |
| | | | CAATCGCGAGGGGCAGGTGACCTTTGCCCAGCGCG |
| | | | CGTTCGCCCCGCCCCGGACGGTATCGATAAGCTTA |
| | | | GGAGCTTGGGCTGCAGGTCGAGGGTTAATCATTAA |
| | | | GTCGTTAATTTTTGTGGCCCTTGCGATGTTTGCTC |
| | | | TGGTTAATAATCTCAGGACAAACAGAGGTTAATAA |
| | | | TTTTCCAGATCTCTCTGAGCAATAGTATAAAAGGC |
| | | | CAGCAGCAGCCTGACCACATCTCATCCTTGAGCCA |
| | | | AGAGGTAAGGGTTTAAGGGATGGTTGGTTGGTGGG |
| | | | GTATTAATGTTTAATTACCTGGAGCACCTGCCTGA |
| | | | AATCACTTTTTTTCAGGTTGGGGATCCGCCACC |
| 27 | L89 | | GAGTTGCTGGTGCTTCCCCAGGCTGGAGATTGAGT |
| | | | TAATATTAACAGGCCCAAGGCGATGTGGGCTTGTG |
| | | | CAATCATAGGCCCGGCCTCGCCAGGTCACCTGAGG |
| | | | AGTTAATGAATACATATCTCCTGAGTTAATAATTA |
| | | | CCAGCGCGGGCCAAATAAATAATCCGCGAGGGGCA |
| | | | GCTGGACTTTGGACTCCGCGCGCTGGTAATTATTA |
| | | | ACCTCGCGAATATTGATTCGAGGCCGCGATTGCCG |
| | | | CAATCGCGAGGGGCAGGTGACCTTTGCCCAGCGCG |
| | | | CGTTCGCCCCGCCCCGGACGGTATCGATAAGCTTA |
| | | | GGAGCTTGGGCTGCAGGTCGAGGGTTAATCATTAA |
| | | | GTCGTTAATTTTTGTGGCCCTTGCGATGTTTGCTC |
| | | | TGGTTAATAATCTCAGGACAAACAGAGGTTAATAA |
| | | | TTTTCCAGATCTCTCTGAGCAATAGTATAAAAGGC |
| | | | CAGCAGCAGCCTGACCACATCTCATCCTGATCCAC |
| | | | AAGCTCGAGCCGAGTGGTCGTTGAGCCAAGAGGTA |
| | | | AGGGTTTAAGGGATGGTTGGTTGGTGGGGTATTAA |
| | | | TGTTTAATTACCTGGAGCACCTGCCTGAAATCACT |
| | | | TTTTTTCAGGTTGGGGATCCGCCACC |
| 28 | LP1v2 | | GAGTTAATAATTACCAGCGCGGGCCAAATAAATAA |
| | | | TCCGCGAGGGGCAGCTGGACTTTGGACTCCGCGCG |
| | | | CTGGTAATTATTAACCTCCCTAAAATGGGCAAACA |
| | | | TTGCAAGCAGCAAACAGCAAACACACAGCCCTCCC |
| | | | TGCCTGCTGACCTTGGAGCTGGGGCAGAGGTCAGA |
| | | | GACCTCTCTGGGCCCATGCCACCTCCAACATCCAC |
| | | | TCGACCCCTTGGAATTTCGGTGGAGAGGAGCAGAG |
| | | | GTTGTCCTGGCGTGGTTTAGGTAGTGTGAGAGGGG |
| | | | AATGACTCCTTTCGGTAAGTGCAGTGGAAGCTGTA |
| | | | CACTGCCCAGGCAAAGCGTCCGGGCAGCGTAGGCG |
| | | | GGCGACTCAGATCCCAGCCAGTGGACTTAGCCCCT |
| | | | GTTTGCTCCTCCGATAACTGGGGTGACCTTGGTTA |
| | | | ATATTCACCAGCAGCCTCCCCCGTTGCCCCTCTGG |
| | | | ATCCACTGCTTAAATACGGACGAGGACAGGGCCCT |
| | | | GTCTCCTCAGCTTCAGGCACCACCACTGACCTGGG |
| | | | ACAGTGAATCGGCGGCCGCCACC |
| 29 | LP1v3 | | GAGTTAATAATTACCAGCGCGGGCCAAATAAATAA |
| | | | TCCGCGAGGGGCAGCTGGACTTTGGACTCCGCGCG |
| | | | CTGGTAATTATTAACCTCGCGAATATTGATTCGAG |
| | | | GCCGCGATTGCCGCAATCGCGAGGGGCAGGTGACC |
| | | | TTTGCCCAGCGGGTACCCGGGGGCGCGCCAGTCCC |
| | | | TAAAATGGGCAAACATTGCAAGCAGCAAACAGCAA |
| | | | ACACACAGCCCTCCCTGCCTGCTGACCTTGGAGCT |
| | | | GGGGCAGAGGTCAGAGACCTCTCTGGGCCCATGCC |
| | | | ACCTCCAACATCCACTCGACCCCTTGGAATTTCGG |
| | | | TGGAGAGGAGCAGAGGTTGTCCTGGCGTGGTTTAG |
| | | | GTAGTGTGAGAGGGGAATGACTCCTTTCGGTAAGT |
| | | | GCAGTGGAAGCTGTACACTGCCCAGGCAAAGCGTC |
| | | | CGGGCAGCGTAGGCGGGCGACTCAGATCCCAGCCA |
| | | | GTGGACTTAGCCCCTGTTTGCTCCTCCGATAACTG |
| | | | GGGTGACCTTGGTTAATATTCACCAGCAGCCTCCC |
| | | | CCGTTGCCCCTCTGGATCCACTGCTTAAATACGGA |
| | | | CGAGGACAGGGCCCTGTCTCCTCAGCTTCAGGCAC |
| | | | CACCACTGACCTGGGACAGTGAATCGGCGGCCGCC |
| | | | ACC |
| 30 | LP1v4 | | GAGTTAATAATTACCAGCGCGGGCCAAATAAATAA |
| | | | TCCGCGCCCTAAAATGGGCAAACATTGCAAGCAGC |
| | | | AAACAGCAAACACACAGCCCTCCCTGCCTGCTGAC |
| | | | CTTGGAGCTGGGGCAGAGGTCAGAGACCTCTCTGG |
| | | | GCCCATGCCACCTCCAACATCCACTCGACCCCTTG |
| | | | GAATTTCGGTGGAGAGGAGCAGAGGTTGTCCTGGC |
| | | | GTGGTTTAGGTAGTGTGAGAGGGGAATGACTCCTT |
| | | | TCGGTAAGTGCAGTGGAAGCTGTACACTGCCCAGG |
| | | | CAAAGCGTCCGGGCAGCGTAGGCGGGCGACTCAGA |
| | | | TCCCAGCCAGTGGACTTAGCCCCTGTTTGCTCCTC |
| | | | CGATAACTGGGGTGACCTTGGTTAATATTCACCAG |
| | | | CAGCCTCCCCCGTTGCCCCTCTGGATCCACTGCTT |
| | | | AAATACGGACGAGGACAGGGCCCTGTCTCCTCAGC |
| | | | TTCAGGCACCACCACTGACCTGGGACAGTGAATCG |
| | | | AAGAGGTAAGGGTTTAAGGGATGGTTGGTTGGTGG |
| | | | GGTATTAATGTTTAATTACCTGGAGCACCTGCCTG |
| | | | AAATCACTTTTTTTCAGGTTGGGCGGCCGCCACC |
| 31 | LP1v5 | | GAGTTAATAATTACCAGCGCGGGCCAAATAAATAA |
| | | | TCCGCGAGGGGCAGCTGGACTTTGGACTCCGCGCG |
| | | | CTGGTAATTATTAACCTCGCGAATATTGATTCGAG |
| | | | GCCGCGATTGCCGCAATCGCGAGGGGCAGGTGACC |
| | | | TTTGCCCAGCGCGCGTTCGCCCCGCCCCGGACCCC |
| | | | TAAAATGGGCAAACATTGCAAGCAGCAAACAGCAA |
| | | | ACACACAGCCCTCCCTGCCTGCTGACCTTGGAGCT |
| | | | GGGGCAGAGGTCAGAGACCTCTCTGGGCCCATGCC |
| | | | ACCTCCAACATCCACTCGACCCCTTGGAATTTCGG |
| | | | TGGAGAGGAGCAGAGGTTGTCCTGGCGTGGTTTAG |
| | | | GTAGTGTGAGAGGGGAATGACTCCTTTCGGTAAGT |
| | | | GCAGTGGAAGCTGTACACTGCCCAGGCAAAGCGTC |
| | | | CGGGCAGCGTAGGCGGGCGACTCAGATCCCAGCCA |
| | | | GTGGACTTAGCCCCTGTTTGCTCCTCCGATAACTG |
| | | | GGGTGACCTTGGTTAATATTCACCAGCAGCCTCCC |
| | | | CCGTTGCCCCTCTGGATCCACTGCTTAAATACGGA |
| | | | CGAGGACAGGGCCCTGTCTCCTCAGCTTCAGGCAC |
| | | | CACCACTGACCTGGGACAGTGAATCGAAGAGGTAA |
| | | | GGGTTTAAGGGATGGTTGGTTGGTGGGGTATTAAT |
| | | | GTTTAATTACCTGGAGCACCTGCCTGAAATCACTT |
| | | | TTTTTCAGGTTGGGCGGCCGCCACC |
| 32 | SynE_ | | GAGTTAATAATTACCAGCGCGGGCCAAATAAATAA |
| | mTTR | | TCCGCGAGGGGCAGGTGACGTTTGCCCAGCGCGCG |
| | | | CTGGTAATTATTAACCTCGCGAATATTGATTCGAG |
| | | | GCCGCGATTGCCGCAATCGCGAGGGGCAGGTGACC |
| | | | TTTGCCCAGCGCGCGTTCGCCCCGCCCCGGACGGT |
| | | | ATCGATAAGCTTAGGAGCTTGGGCTGCAGGTCGAG |
| | | | GGCACTGGGAGGATGTTGAGTAAGATGGAAAACTA |
| | | | CTGATGACCCTTGCAGAGACAGAGTATTAGGACAT |
| | | | GTTTGAACAGGGGCCGGGCGATCAGCAGGTAGCTC |
| | | | TAGAGGATCCCCGTCTGTCTGCACATTTCGTAGAG |
| | | | CGAGTGTTCCGATACTCTAATCTCCCTAGGCAAGG |
| | | | TTCATATTTGTGTAGGTTACTTATTCTCCTTTTGT |
| | | | TGACTAAGTCAATAATCAGAATCAGCAGGTTTGGA |
| | | | GTCAGCTTGGCAGGGATCAGCAGCCTGGGTTGGAA |
| | | | GGAGGGGGTATAAAAGCCCCTTCACCAGGAGAAGC |
| | | | CGTCACACAGATCCACAAGCTCCTGCGGCCGCCAC |
| | | | C |
| 33 | SynE_ | | GAGTTAATAATTACCAGCGCGGGCCAAATAAATAA |
| | mTTR_ | | TCCGCGAGGGGCAGGTGACGTTTGCCCAGCGCGCG |
| | TISU | | CTGGTAATTATTAACCTCGCGAATATTGATTCGAG |
| | | | GCCGCGATTGCCGCAATCGCGAGGGGCAGGTGACC |
| | | | TTTGCCCAGCGCGCGTTCGCCCCGCCCCGGACGGT |
| | | | ATCGATAAGCTTAGGAGCTTGGGCTGCAGGTCGAG |
| | | | GGCACTGGGAGGATGTTGAGTAAGATGGAAAACTA |
| | | | CTGATGACCCTTGCAGAGACAGAGTATTAGGACAT |
| | | | GTTTGAACAGGGGCCGGGCGATCAGCAGGTAGCTC |
| | | | TAGAGGATCCCCGTCTGTCTGCACATTTCGTAGAG |
| | | | CGAGTGTTCCGATACTCTAATCTCCCTAGGCAAGG |
| | | | TTCATATTTGTGTAGGTTACTTATTCTCCTTTTGT |
| | | | TGACTAAGTCAATAATCAGAATCAGCAGGTTTGGA |
| | | | GTCAGCTTGGCAGGGATCAGCAGCCTGGGTTGGAA |
| | | | GGAGGGGGTATAAAAGCCCCTTCACCAGGAGAAGC |
| | | | CGTCACACAGATCCACAAGCTCCTGCGCGGCCGCA |
| | | | AG |

TABLE 9
ATP7B minigene sequences.

| SEQ | | |
|---|---|---|
| ID | | |
| NO | Name | Sequence |
| 34 | ATP7B_FL | ATGCCTGAACAGGAGAGACAGATCACAGCCAGAGAAGGGGCCAG |
| | | TCGGAAAATCTTATCTAAGCTTTCTTTGCCTACCCGTGCCTGGGAA |
| | | CCAGCAATGAAGAAGAGTTTTGCTTTTGACAATGTTGGCTATGAA |
| | | GGTGGTCTGGATGGCCTGGGCCCTTCTTCTCAGGTGGCCACCAGC |
| | | ACAGTCAGGATCTTGGGCATGACTTGCCAGTCATGTGTGAAGTCC |
| | | ATTGAGGACAGGATTTCCAATTTGAAAGGCATCATCAGCATGAAG |
| | | GTTTCCCTGGAACAAGGCAGTGCCACTGTGAAATATGTGCCATCG |
| | | GTTGTGTGCCTGCAACAGGTTTGCCATCAAATTGGGGACATGGGC |
| | | TTCGAGGCCAGCATTGCAGAAGGAAAGGCAGCCTCCTGGCCCTCA |
| | | AGGTCCTTGCCTGCCCAGGAGGCTGTGGTCAAGCTCCGGGTGGAG |
| | | GGCATGACCTGCCAGTCCTGTGTCAGCTCCATTGAAGGCAAGGTC |
| | | CGGAAACTGCAAGGAGTAGTGAGAGTCAAAGTCTCACTCAGCAA |
| | | CCAAGAGGCCGTCATCACTTATCAGCCTTATCTCATTCAGCCCGA |
| | | AGACCTCAGGGACCATGTAAATGACATGGGATTTGAAGCTGCCAT |
| | | CAAGAGCAAAGTGGCTCCCTTAAGCCTGGGACCAATTGATATTGA |
| | | GCGGTTACAAAGCACTAACCCAAAGAGACCTTTATCTTCTGCTAA |
| | | CCAGAATTTTAATAATTCTGAGACCTTGGGGCACCAAGGAAGCCA |
| | | TGTGGTCACCCTCCAACTGAGAATAGATGGAATGCATTGTAAGTC |
| | | TTGCGTCTTGAATATTGAAGAAAATATTGGCCAGCTCCTAGGGGT |
| | | TCAAAGTATTCAAGTCTCCTTGGAGAACAAAACTGCCCAAGTAAA |
| | | GTATGACCCTTCTTGTACCAGCCCAGTGGCTCTGCAGAGGGCTAT |
| | | CGAGGCACTTCCACCTGGGAATTTTAAAGTTTCTCTTCCTGATGGA |
| | | GCCGAAGGGAGTGGGACAGATCACAGGTCTTCCAGTTCTCATTCC |
| | | CCTGGCTCCCCACCGAGAAACCAGGTCCAGGGCACATGCAGTACC |
| | | ACTCTGATTGCCATTGCCGGCATGACCTGTGCATCCTGTGTCCATT |
| | | CCATTGAAGGCATGATCTCCCAACTGGAAGGGGTGCAGCAAATAT |
| | | CGGTGTCTTTGGCCGAAGGGACTGCAACAGTTCTTTATAATCCCG |
| | | CTGTAATTAGCCCAGAAGAACTCAGAGCTGCTATAGAAGACATGG |
| | | GATTTGAGGCTTCAGTCGTTTCTGAAAGCTGTTCTACTAACCCTCT |
| | | TGGAAACCACAGTGCTGGGAATTCCATGGTGCAAACTACAGATGG |
| | | TACACCTACATCTCTGCAGGAAGTGGCTCCCCACACTGGGAGGCT |
| | | CCCTGCAAACCATGCCCCGGACATCTTGGCAAAGTCCCCACAATC |
| | | AACCAGAGCAGTGGCACCGCAGAAGTGCTTCTTACAGATCAAAG |
| | | GCATGACCTGTGCATCCTGTGTGTCTAACATAGAAAGGAATCTGC |
| | | AGAAAGAAGCTGGTGTTCTCTCCGTGTTGGTTGCCTTGATGGCAG |
| | | GAAAGGCAGAGATCAAGTATGACCCAGAGGTCATCCAGCCCCTC |
| | | GAGATAGCTCAGTTCATCCAGGACCTGGGTTTTGAGGCAGCAGTC |
| | | ATGGAGGACTACGCAGGCTCCGATGGCAACATTGAGCTGACAATC |
| | | ACAGGGATGACCTGCGCGTCCTGTGTCCACAACATAGAGTCCAAA |
| | | CTCACGAGGACAAATGGCATCACTTATGCCTCCGTTGCCCTTGCC |
| | | ACCAGCAAAGCCCTTGTTAAGTTTGACCCGGAAATTATCGGTCCA |
| | | CGGGATATTATCAAAATTATTGAGGAAATTGGCTTTCATGCTTCCC |
| | | TGGCCCAGAGAAACCCCAACGCTCATCACTTGGACCACAAGATGG |
| | | AAATAAAGCAGTGGAAGAAGTCTTTCCTGTGCAGCCTGGTGTTTG |
| | | GCATCCCTGTCATGGCCTTAATGATCTATATGCTGATACCCAGCA |
| | | ACGAGCCCCACCAGTCCATGGTCCTGGACCACAACATCATTCCAG |
| | | GACTGTCCATTCTAAATCTCATCTTCTTTATCTTGTGTACCTTTGTC |
| | | CAGCTCCTCGGTGGGTGGTACTTCTACGTTCAGGCCTACAAATCTC |
| | | TGAGACACAGGTCAGCCAACATGGACGTGCTCATCGTCCTGGCCA |
| | | CAAGCATTGCTTATGTTTATTCTCTGGTCATCCTGGTGGTTGCTGT |
| | | GGCTGAGAAGGCGGAGAGGAGCCCTGTGACATTCTTCGACACGC |
| | | CCCCCATGCTCTTTGTGTTCATTGCCCTGGGCCGGTGGCTGGAACA |
| | | CTTGGCAAAGAGCAAAACCTCAGAAGCCCTGGCTAAACTCATGTC |
| | | TCTCCAAGCCACAGAAGCCACCGTTGTGACCCTTGGTGAGGACAA |
| | | TTTAATCATCAGGGAGGAGCAAGTCCCCATGGAGCTGGTGCAGCG |
| | | GGGCGATATCGTCAGGGTGGTCCCTGGGGGAAAGTTTCCAGTGGA |
| | | TGGGAAAGTCCTGGAAGGCAATACCATGGCTGATGAGTCCCTCAT |
| | | CACAGGAGAAGCCATGCCAGTCACTAAGAAACCCGGAAGCACTG |
| | | TAATTGCGGGGTCTATAAATGCACATGGCTCTGTGCTCATTAAAG |
| | | CTACCCACGTGGGCAATGACACCACTTTGGCTCAGATTGTGAAAC |
| | | TGGTGGAAGAGGCTCAGATGTCAAAGGCACCCATTCAGCAGCTG |
| | | GCTGACCGGTTTAGTGGATATTTTGTCCCATTTATCATCATCATGT |
| | | CAACTTTGACGTTGGTGGTATGGATTGTAATCGGTTTTATCGATTT |
| | | TGGTGTTGTTCAGAAATACTTTCCTAACCCCAACAAGCACATCTCC |
| | | CAGACAGAGGTGATCATCCGGTTTGCTTTCCAGACGTCCATCACG |
| | | GTGCTGTGCATTGCCTGCCCCTGCTCCCTGGGGCTGGCCACGCCC |
| | | ACGGCTGTCATGGTGGGCACCGGGGTGGCCGCGCAGAACGGCAT |
| | | CCTCATCAAGGGAGGCAAGCCCCTGGAGATGGCGCACAAGATAA |
| | | AGACTGTGATGTTTGACAAGACTGGCACCATTACCCATGGCGTCC |
| | | CCAGGGTCATGCGGGTGCTCCTGCTGGGGGATGTGGCCACACTGC |
| | | CCCTCAGGAAGGTTCTGGCTGTGGTGGGGACTGCGGAGGCCAGCA |
| | | GTGAACACCCCTTGGGCGTGGCAGTCACCAAATACTGTAAAGAGG |
| | | AACTTGGAACAGAGACCTTGGGATACTGCACGGACTTCCAGGCAG |
| | | TGCCAGGCTGTGGAATTGGGTGCAAAGTCAGCAACGTGGAAGGC |
| | | ATCCTGGCCCACAGTGAGCGCCCTTTGAGTGCACCGGCCAGTCAC |
| | | CTGAATGAGGCTGGCAGCCTTCCCGCAGAAAAAGATGCAGCCCCC |
| | | CAGACCTTCTCTGTGCTGATTGGAAACCGTGAGTGGCTGAGGCGC |
| | | AACGGTTTAACCATTTCTAGCGATGTCAGTGACGCTATGACAGAC |
| | | CACGAGATGAAAGGACAGACAGCCATCCTGGTGGCTATTGACGG |
| | | TGTGCTCTGTGGGATGATCGCAATCGCAGACGCTGTCAAGCAGGA |
| | | GGCTGCCCTGGCTGTGCACACGCTGCAGAGCATGGGTGTGGACGT |
| | | GGTTCTGATCACGGGGGACAACCGGAAGACAGCCAGAGCTATTG |
| | | CCACCCAGGTTGGCATCAACAAAGTCTTTGCAGAGGTGCTGCCTT |
| | | CGCACAAGGTGGCCAAGGTCCAGGAGCTCCAGAATAAAGGGAAG |
| | | AAAGTCGCCATGGTGGGGGATGGGGTCAATGACTCCCCGGCCTTG |
| | | GCCCAGGCAGACATGGGTGTGGCCATTGGCACCGGCACGGATGT |
| | | GGCCATCGAGGCAGCCGACGTCGTCCTTATCAGAAATGATTTGCT |
| | | GGATGTGGTGGCTAGCATTCACCTTTCCAAGAGGACTGTCCGAAG |
| | | GATACGCATCAACCTGGTCCTGGCACTGATTTATAACCTGGTTGG |
| | | GATACCCATTGCAGCAGGTGTCTTCATGCCCATCGGCATTGTGCT |
| | | GCAGCCCTGGATGGGCTCAGCGGCCATGGCAGCCTCCTCTGTGTC |
| | | TGTGGTGCTCTCATCCCTGCAGCTCAAGTGCTATAAGAAGCCTGA |
| | | CCTGGAGAGGTATGAGGCACAGGCGCATGGCCACATGAAGCCCC |
| | | TGACGGCATCCCAGGTCAGTGTGCACATAGGCATGGATGACAGGT |
| | | GGCGGGACTCCCCCAGGGCCACACCATGGGACCAGGTCAGCTAT |
| | | GTCAGCCAGGTGTCGCTGTCCTCCCTGACGTCCGACAAGCCATCT |
| | | CGGCACAGCGCTGCAGCAGACGATGATGGGGACAAGTGGTCTCT |
| | | GCTCCTGAATGGCAGGGATGAGGAGCAGTACATCTGA |
| 35 | miniATP7AB | ATGGCGGCGCCTGAGCAGGAGAGACAGATCACAGCCAGAGAAGG |
| | | GGCCAGTCGGAAAATCTTATCTAAGCTTTCTTTGCCTACCCGTGCC |
| | | TGGGAACCAGCAATGAAGAAGAGTTTTGCTTTTGACAATGTTGGC |
| | | TATGAAGGTGGTCTGGATGGCCTGGGCCCTTCTTCTCAGGTGGCC |
| | | CCTGATGGAGCCGAAGGGAGTGGGACAGATCACAGGTCTTCCAG |
| | | TTCTCATTCCCCTGGCTCCCCACCGAGAAACCAGGTCCAGGGCAC |
| | | ATGCAGTAAGTGCTTCTTACAGATCAAAGGCATGACCTGTGCATC |
| | | CTGTGTGTCTAACATAGAAAGGAATCTGCAGAAAGAAGCTGGTGT |
| | | TCTCTCCGTGTTGGTTGCCTTGATGGCAGGAAAGGCAGAGATCAA |
| | | GTATGACCCAGAGGTCATCCAGCCCCTCGAGATAGCTCAGTTCAT |
| | | CCAGGACCTGGGTTTTGAGGCAGCAGTCATGGAGGACTACGCAG |
| | | GCTCCGATGGCAACATTGAGCTGACAATCACAGGGATGACCTGCG |
| | | CGTCCTGTGTCCACAACATAGAGTCCAAACTCACGAGGACAAATG |
| | | GCATCACTTATGCCTCCGTTGCCCTTGCCACCAGCAAAGCCCTTGT |
| | | TAAGTTTGACCCGGAAATTATCGGTCCACGGGATATTATCAAAAT |
| | | TATTGAGGAAATTGGCTTTCATGCTTCCCTGGCCCAGAGAAACCC |
| | | CAACGCTCATCACTTGGACCACAAGATGGAAATAAAGCAGTGGA |
| | | AGAAGTCTTTTCTTGTGAGTCTGTTTTTCTGTATTCCTGTAATGGG |
| | | GCTGATGATATATATGATGGTTATGGACCACCACTTTGCAACTCTT |
| | | CACCATAATCAAAACATGAGTAAAGAAGAAATGATCAACCTTCAT |
| | | TCTTCTATGTTCCTGGAGCGCCAGATTCTTCCAGGATTGTCTGTTA |
| | | TGAATTTGCTGTCCTTTTTATTGTGTGTACCTGTACAGTTTTTCGGA |
| | | GGCTGGTACTTCTACATTCAGGCTTATAAAGCACTGAAGCATAAG |
| | | ACAGCAAATATGGACGTACTGATTGTGCTGGCAACCACCATTGCA |
| | | TTTGCCTACTCTTTGATTATTCTTCTAGTTGCAATGTATGAGAGAG |
| | | CCAAAGTGAACCCTATTACTTTCTTTGACACACCCCCTATGCTGTT |
| | | TGTGTTTATTGCACTAGGCCGATGGCTGGAACATATAGCAAAGGG |
| | | CAAAACATCAGAGGCTCTTGCAAAGTTAATTTCACTACAAGCTAC |
| | | AGAAGCAACTATTGTAACTCTTGATTCTGATAATATCCTCCTCAGT |
| | | GAAGAACAAGTGGATGTGGAACTTGTACAACGTGGAGATATCATT |
| | | AAAGTAGTTCCAGGAGGCAAATTTCCAGTGGATGGTCGTGTTATT |
| | | GAAGGACATTCTATGGTAGATGAGTCCCTCATCACAGGGGAGGCA |
| | | ATGCCTGTGGCTAAGAAACCTGGCAGCACAGTGATTGCTGGTTCC |
| | | ATTAACCAGAACGGGTCACTGCTTATCTGCGCAACACATGTTGGA |
| | | GCAGACACAACCCTTTCTCAAATTGTCAAACTTGTGGAAGAGGCA |
| | | CAAACATCAAAGGCTCCTATCCAGCAGTTTGCAGACAAACTCAGT |
| | | GGCTATTTTGTTCCTTTTATTGTTTTTGTTTCCATTGCCACCCTCTT |
| | | GGTATGGATTGTAATTGGATTTCTGAATTTTGAAATTGTGGAAAC |
| | | CTACTTTCCTGGCTACAATAGAAGTATCTCCCGAACAGAAACGAT |
| | | AATACGATTTGCTTTCCAAGCCTCTATCACAGTTCTGTGTATTGCA |
| | | TGTCCCTGTTCACTGGGACTGGCCACTCCAACTGCTGTGATGGTG |
| | | GGTACAGGAGTAGGTGCTCAAAATGGCATACTAATAAAAGGTGG |
| | | AGAGCCATTGGAGATGGCTCATAAGGTAAAGGTAGTGGTATTTGA |
| | | TAAGACTGGAACCATTACTCACGGAACCCCAGTGGTGAATCAAGT |
| | | AAAGGTTCTAACTGAAAGTAACAGAATATCACACCATAAAATCTT |
| | | GGCCATTGTGGGAACTGCTGAAAGTAACAGTGAACACCCTCTAGG |
| | | AACAGCCATAACCAAATATTGCAAACAGGAGCTGGACACTGAAA |
| | | CCTTGGGTACCTGCATAGATTTCCAGGTTGTGCCAGGCTGTGGTAT |
| | | TAGCTGTAAAGTCACCAATATTGAAGGCTTGCTACATAAGAATAA |
| | | CTGGAATATAGAGGACAATAATATTAAAAATGCATCCCTGGTTCA |
| | | AATTGATGCCAGTAATGAACAGTCATCAACTTCGTCTTCCATGATT |
| | | ATTGATGCCCAGATCTCAAATGCTCTTAATGCTCAGCAGTATAAA |
| | | GTCCTCATTGGTAACCGGGAGTGGATGATTAGAAATGGTCTTGTC |
| | | ATTAATAACGATGTAAATGATTTCATGACTGAACATGAGAGAAAA |
| | | GGTCGGACTGCTGTATTAGTAGCAGTTGATGATGAGCTGTGTGGC |
| | | TTGATAGCCATTGCAGACACAGTGAAGCCTGAAGCAGAACTGGCT |
| | | ATCCATATTCTGAAATCTATGGGCTTAGAAGTAGTTCTGATGACT |
| | | GGAGACAACAGTAAAACAGCTAGATCTATTGCTTCTCAGGTTGGC |
| | | ATTACTAAGGTGTTTGCTGAAGTTCTACCTTCTCACAAGGTTGCTA |
| | | AAGTGAAGCAACTTCAAGAGGAGGGGAAACGGGTAGCAATGGTG |
| | | GGAGATGGAATCAATGACTCCCCAGCTCTGGCAATGGCTAATGTG |
| | | GGAATTGCTATTGGCACAGGCACAGATGTAGCCATTGAAGCAGCT |
| | | GATGTGGTTTTGATAAGGAATGATCTTCTGGATGTAGTGGCAAGT |
| | | ATTGACTTATCAAGAAAGACAGTCAAGAGGATTCGGATAAATTTT |
| | | GTCTTTGCTCTAATTTATAATCTGGTTGGAATTCCCATAGCTGCTG |
| | | GAGTTTTTATGCCCATTGGTTTGGTTTTGCAGCCCTGGATGGGATC |
| | | TGCAGCAATGGCTGCTTCATCTGTTTCTGTAGTACTTTCTTCTCTCC |
| | | AGCTCAAGTGCTATAAGAAGCCTGACCTGGAGAGGTATGAGGCA |
| | | CAGGCGCATGGCCACATGAAGCCCCTGACGGCATCCCAGGTCAGT |
| | | GTGCACATAGGCATGGATGACAGGTGGCGGGACTCCCCCAGGGC |
| | | CACACCATGGGACCAGGTCAGCTATGTCAGCCAGGTGTCGCTGTC |
| | | CTCCCTGACGTCCGACAAGCCATCTCGGCACAGCGCTGCAGCAGA |
| | | CGATGATGGGGACAAGTGGTCTCTGCTCCTGAATGGCAGGGATGA |
| | | GGAGCAGTACATCTGA |
| 36 | miniATP7B- | ATGCCTGAGCAGGAGAGACAGATCACAGCCAGAGAAGGGGCCAG |
| | S1 | TCGGAAAATCTTATCTAAGCTTTCTTTGCCTACCCGTGCCTGGGAA |
| | | CCAGCAATGAAGAAGAGTTTTGCTTTTGACAATGTTGGCTATGAA |
| | | GGTGGTCTGGATGGCCTGGGCCCTTCTTCTCAGGTGGCCACCACT |
| | | CTGATTGCCATTGCCGGCATGACCTGTGCATCCTGTGTCCATTCCA |
| | | TTGAAGGCATGATCTCCCAACTGGAAGGGGTGCAGCAAATATCGG |
| | | TGTCTTTGGCCGAAGGGACTGCAACAGTTCTTTATAATCCCTCTGT |
| | | AATTAGCCCAGAAGAACTCAGAGCTGCTATAGAAGACATGGGAT |
| | | TTGAGGCTTCAGTCGTTTCTGAAAGCTGTTCTACTAACCCTCTTGG |
| | | AAACCACAGTGCTGGGAATTCCATGGTGCAAACTACAGATGGTAC |
| | | ACCTACATCTGTGCAGGAAGTGGCTCCCCACACTGGGAGGCTCCC |
| | | TGCAAACCATGCCCCGGACATCTTGGCAAAGTCCCCACAATCAAC |
| | | CAGAGCAGTGGCACCGCAGAAGTGCTTCTTACAGATCAAAGGCAT |
| | | GACCTGTGCATCCTGTGTGTCTAACATAGAAAGGAATCTGCAGAA |
| | | AGAAGCTGGTGTTCTCTCCGTGTTGGTTGCCTTGATGGCAGGAAA |
| | | GGCAGAGATCAAGTATGACCCAGAGGTCATCCAGCCCCTCGAGAT |
| | | AGCTCAGTTCATCCAGGACCTGGGTTTTGAGGCAGCAGTCATGGA |
| | | GGACTACGCAGGCTCCGATGGCAACATTGAGCTGACAATCACAG |
| | | GGATGACCTGCGCGTCCTGTGTCCACAACATAGAGTCCAAACTCA |
| | | CGAGGACAAATGGCATCACTTATGCCTCCGTTGCCCTTGCCACCA |
| | | GCAAAGCCCTTGTTAAGTTTGACCCGGAAATTATCGGTCCACGGG |
| | | ATATTATCAAAATTATTGAGGAAATTGGCTTTCATGCTTCCCTGGC |
| | | CCAGAGAAACCCCAACGCTCATCACTTGGACCACAAGATGGAAA |
| | | TAAAGCAGTGGAAGAAGTCTTTCCTGTGCAGCCTGGTGTTTGGCA |
| | | TCCCTGTCATGGCCTTAATGATCTATATGCTGATACCCAGCAACG |
| | | AGCCCCACCAGTCCATGGTCCTGGACCACAACATCATTCCAGGAC |
| | | TGTCCATTCTAAATCTCATCTTCTTTATCTTGTGTACCTTTGTCCAG |
| | | CTCCTCGGTGGGTGGTACTTCTACGTTCAGGCCTACAAATCTCTGA |
| | | GACACAGGTCAGCCAACATGGACGTGCTCATCGTCCTGGCCACAA |
| | | GCATTGCTTATGTTTATTCTCTGGTCATCCTGGTGGTTGCTGTGGC |
| | | TGAGAAGGCGGAGAGGAGCCCTGTGACATTCTTCGACACGCCCCC |
| | | CATGCTCTTTGTGTTCATTGCCCTGGGCCGGTGGCTGGAACACTTG |
| | | GCAAAGAGCAAAACCTCAGAAGCCCTGGCTAAACTCATGTCTCTC |
| | | CAAGCCACAGAAGCCACCGTTGTGACCCTTGGTGAGGACAATTTA |
| | | ATCATCAGGGAGGAGCAAGTCCCCATGGAGCTGGTGCAGCGGGG |
| | | CGATATCGTCAAGGTGGTCCCTGGGGGAAAGTTTCCAGTGGATGG |
| | | GAAAGTCCTGGAAGGCAATACCATGGCTGATGAGTCCCTCATCAC |
| | | AGGAGAAGCCATGCCAGTCACTAAGAAACCCGGAAGCACTGTAA |
| | | TTGCGGGGTCTATAAATGCACATGGCTCTGTGCTCATTAAAGCTA |
| | | CCCACGTGGGCAATGACACCACTTTGGCTCAGATTGTGAAACTGG |
| | | TGGAAGAGGCTCAGATGTCAAAGGCACCCATTCAGCAGCTGGCTG |
| | | ACCGGTTTAGTGGATATTTTGTCCCATTTATCATCATCATGTCAAC |
| | | TTTGACGTTGGTGGTATGGATTGTAATCGGTTTTATCGATTTTGGT |
| | | GTTGTTCAGAGATACTTTCCTAACCCCAACAAGCACATCTCCCAG |
| | | ACAGAGGTGATCATCCGGTTTGCTTTCCAGACGTCCATCACGGTG |
| | | CTGTGCATTGCCTGCCCCTGCTCCCTGGGGCTGGCCACGCCCACG |
| | | GCTGTCATGGTGGGCACCGGGGTGGCCGCGCAGAACGGCATCCTC |
| | | ATCAAGGGAGGCAAGCCCCTGGAGATGGCGCACAAGATAAAGAC |
| | | TGTGATGTTTGACAAGACTGGCACCATTACCCATGGCGTCCCCAG |
| | | GGTCATGCGGGTGCTCCTGCTGGGGGATGTGGCCACACTGCCCCT |
| | | CAGGAAGGTTCTGGCTGTGGTGGGGACTGCGGAGGCCAGCAGTG |
| | | AACACCCCTTGGGCGTGGCAGTCACCAAATACTGTAAAGAGGAA |
| | | CTTGGAACAGAGACCTTGGGATACTGCACGGACTTCCAGGCAGTG |
| | | CCAGGCTGTGGAATTGGGTGCAAAGTCAGCAACGTGGAAGGCAT |
| | | CCTGGCCCACAGTGAGCGCCCTTTGAGTGCACCGGCCAGTCACCT |
| | | GAATGAGGCTGGCAGCCTTCCCGCAGAAAAAGATGCAGTCCCCC |
| | | AGACCTTCTCTGTGCTGATTGGAAACCGTGAGTGGCTGAGGCGCA |
| | | ACGGTTTAACCATTTCTAGCGATGTCAGTGACGCTATGACAGACC |
| | | ACGAGATGAAAGGACAGACAGCCATCCTGGTGGCTATTGACGGT |
| | | GTGCTCTGTGGGATGATCGCAATCGCAGACGCTGTCAAGCAGGAG |
| | | GCTGCCCTGGCTGTGCACACGCTGCAGAGCATGGGTGTGGACGTG |
| | | GTTCTGATCACGGGGGACAACCGGAAGACAGCCAGAGCTATTGC |
| | | CACCCAGGTTGGCATCAACAAAGTCTTTGCAGAGGTGCTGCCTTC |
| | | GCACAAGGTGGCCAAGGTCCAGGAGCTCCAGAATAAAGGGAAGA |
| | | AAGTCGCCATGGTGGGGGATGGGGTCAATGACTCCCCGGCCTTGG |
| | | CCCAGGCAGACATGGGTGTGGCCATTGGCACCGGCACGGATGTG |
| | | GCCATCGAGGCAGCCGACGTCGTCCTTATCAGAAATGATTTGCTG |
| | | GATGTGGTGGCTAGCATTCACCTTTCCAAGAGGACTGTCCGAAGG |
| | | ATACGCATCAACCTGGTCCTGGCACTGATTTATAACCTGGTTGGG |
| | | ATACCCATTGCAGCAGGTGTCTTCATGCCCATCGGCATTGTGCTGC |
| | | AGCCCTGGATGGGCTCAGCGGCCATGGCAGCCTCCTCTGTGTCTG |
| | | TGGTGCTCTCATCCCTGCAGCTCAAGTGCTATAAGAAGCCTGACC |
| | | TGGAGAGGTATGAGGCACAGGCGCATGGCCACATGAAGCCCCTG |
| | | ACGGCATCCCAGGTCAGTGTGCACATAGGCATGGATGACAGGTG |
| | | GCGGGACTCCCCCAGGGCCACACCATGGGACCAGGTCAGCTATGT |
| | | CAGCCAGGTGTCGCTGTCCTCCCTGACGTCCGACAAGCCATCTCG |
| | | GCACAGCGCTGCAGCAGACGATGATGGGGACAAGTGGTCTCTGCT |
| | | CCTGAATGGCAGGGATGAGGAGCAGTACATCTGA |
| 37 | miniATP7B- | ATGCCGGAACAAGAACGCCAGATCACAGCAAGAGAAGGAGCAAG |
| | s1co | TAGGAAGATCCTGAGCAAGCTCTCCCTGCCCACAAGGGCCTGGGA |
| | | GCCGGCCATGAAGAAGTCTTTTGCCTTCGATAATGTCGGTTATGA |
| | | AGGCGGGCTCGATGGCCTGGGCCCCAGCAGCCAGGTGGCGACCA |
| | | CCTTAATCGCCATCGCAGGCATGACCTGCGCATCTTGTGTGCATA |
| | | GCATTGAAGGCATGATTAGCCAGCTGGAGGGCGTGCAGCAGATCT |
| | | CAGTCTCCCTGGCTGAGGGGACCGCCACTGTCTTGTACAATCCGT |
| | | CTGTTATTTCCCCGGAGGAGCTGAGAGCTGCCATTGAGGACATGG |
| | | GATTTGAAGCATCGGTGGTGTCTGAGTCATGCTCCACCAATCCCC |
| | | TGGGAAATCACTCAGCTGGCAATTCAATGGTTCAGACCACCGATG |
| | | GGACCCCAACCAGCGTGCAGGAAGTGGCACCCCATACCGGCAGA |
| | | CTGCCTGCCAATCATGCTCCTGATATCCTCGCCAAGAGCCCCCAG |
| | | AGCACCCGCGCGGTGGCACCCCAGAAGTGTTTCCTGCAGATCAAG |
| | | GGCATGACCTGTGCCAGCTGTGTTAGCAATATCGAAAGAAACCTC |
| | | CAAAAAGAGGCTGGAGTTCTGAGTGTTCTAGTGGCTTTAATGGCC |
| | | GGGAAAGCCGAGATCAAGTATGATCCCGAGGTGATTCAGCCATTG |
| | | GAGATTGCCCAGTTCATCCAGGATCTGGGCTTCGAGGCCGCCGTG |
| | | ATGGAGGATTACGCCGGCTCTGATGGCAACATCGAACTGACCATC |
| | | ACCGGCATGACCTGTGCTTCCTGCGTGCATAATATCGAGAGCAAA |
| | | CTGACGCGGACCAATGGAATCACCTATGCCAGCGTCGCCCTGGCA |
| | | ACCTCTAAGGCTCTGGTGAAGTTTGACCCCGAAATCATTGGACCT |
| | | AGAGACATCATCAAAATTATTGAAGAGATTGGTTTCCACGCCAGC |
| | | TTAGCTCAGCGGAATCCCAACGCACACCATCTTGACCATAAGATG |
| | | GAAATCAAACAGTGGAAGAAGAGCTTCTTGTGTAGCCTGGTCTTC |
| | | GGGATACCCGTCATGGCACTGATGATTTACATGCTCATTCCGTCC |
| | | AACGAGCCCCACCAGTCCATGGTGCTGGACCACAACATCATCCCT |
| | | GGGCTGTCTATCCTCAACCTGATCTTCTTTATTCTGTGCACATTTG |
| | | TCCAGCTCTTGGGAGGCTGGTATTTCTATGTACAGGCCTACAAAT |
| | | CCCTGAGGCATCGAAGCGCCAACATGGACGTTTTAATTGTGCTGG |
| | | CTACATCCATTGCCTACGTGTACTCCCTGGTGATCTTAGTGGTTGC |
| | | TGTGGCTGAAAAGGCTGAGAGAAGCCCTGTCACCTTCTTCGACAC |
| | | CCCCCCCATGCTCTTCGTGTTTATTGCACTGGGTCGCTGGCTGGAG |
| | | CATTTAGCCAAGTCCAAGACATCCGAGGCTCTGGCCAAGCTGATG |
| | | TCTCTACAGGCCACGGAGGCCACCGTGGTGACTCTGGGTGAAGAC |
| | | AATTTGATAATACGCGAGGAACAGGTGCCTATGGAGCTTGTGCAG |
| | | CGCGGAGATATCGTCAAGGTGGTGCCCGGCGGCAAGTTCCCAGTG |
| | | GATGGAAAAGTGTTAGAGGGCAATACCATGGCCGATGAATCCCTC |
| | | ATCACTGGCGAGGCAATGCCGGTGACCAAGAAACCTGGAAGTAC |
| | | TGTCATTGCAGGCAGCATCAATGCCCATGGGAGCGTCCTGATCAA |
| | | GGCCACCCACGTGGGCAATGATACAACTCTCGCCCAGATCGTCAA |
| | | GCTAGTGGAAGAGGCTCAGATGTCTAAAGCTCCAATCCAGCAACT |
| | | GGCTGACCGCTTCAGCGGCTACTTTGTCCCCTTTATCATCATCATG |
| | | AGCACCCTCACCCTGGTGGTGTGGATAGTCATAGGCTTCATTGAC |
| | | TTTGGCGTGGTGCAGAGATACTTCCCCAACCCTAACAAACATATC |
| | | TCCCAAACCGAGGTCATTATCAGATTCGCCTTTCAGACATCTATCA |
| | | CTGTGCTGTGCATAGCCTGCCCTTGCTCTCTGGGCCTGGCCACCCC |
| | | CACCGCCGTGATGGTTGGAACCGGGGTCGCTGCACAAAATGGGAT |
| | | CCTGATAAAAGGGGGCAAACCCCTGGAAATGGCACATAAGATTA |
| | | AAACAGTTATGTTTGATAAGACAGGCACCATTACTCACGGTGTCC |
| | | CTCGAGTCATGAGAGTGCTGCTGCTGGGGGACGTGGCTACACTGC |
| | | CACTGCGCAAGGTGCTGGCTGTGGTGGGCACAGCGGAGGCCTCCT |
| | | CAGAACACCCATTAGGCGTAGCTGTCACAAAATACTGTAAGGAG |
| | | GAGCTGGGGACAGAGACCCTCGGCTATTGTACAGACTTCCAGGCC |
| | | GTGCCTGGCTGCGGCATCGGCTGTAAGGTCAGCAATGTGGAGGGC |
| | | ATCCTGGCACACAGCGAGAGACCCCTCTCTGCCCCAGCCTCCCAC |
| | | CTGAATGAAGCTGGGTCTCTGCCTGCTGAGAAGGACGCCGTCCCT |
| | | CAGACCTTCTCCGTGTTGATTGGGAACAGAGAGTGGCTTAGAAGG |
| | | AATGGCCTGACCATCAGCTCTGACGTCTCGGATGCCATGACAGAT |
| | | CACGAGATGAAAGGCCAGACAGCCATACTGGTTGCCATCGACGG |
| | | CGTGCTCTGTGGCATGATTGCCATCGCTGATGCTGTTAAACAGGA |
| | | AGCTGCGCTGGCAGTCCACACCCTCCAGTCCATGGGAGTGGACGT |
| | | GGTCCTGATTACTGGAGACAACAGGAAAACCGCTCGGGCCATCGC |
| | | TACCCAAGTGGGGATAAATAAGGTTTTTGCAGAAGTCTTACCCTC |
| | | CCACAAAGTAGCCAAAGTGCAGGAGCTCCAGAACAAGGGTAAAA |
| | | AGGTGGCAATGGTGGGAGATGGTGTGAATGACAGCCCAGCTCTC |
| | | GCGCAGGCTGACATGGGCGTAGCCATAGGTACTGGAACAGATGT |
| | | CGCGATTGAAGCCGCTGACGTAGTCCTCATCCGAAACGATTTGCT |
| | | GGATGTGGTGGCCAGCATCCATCTGTCCAAGCGTACCGTGCGCCG |
| | | GATCCGCATCAACCTGGTCTTGGCCCTCATCTATAATCTCGTGGGC |
| | | ATCCCCATCGCTGCTGGGGTCTTCATGCCCATCGGCATTGTGCTGC |
| | | AGCCCTGGATGGGAAGCGCTGCCATGGCTGCTTCTTCTGTCTCCGT |
| | | TGTGCTGTCCAGCCTACAGCTGAAGTGCTATAAAAAGCCAGACCT |
| | | CGAGCGCTACGAAGCCCAGGCGCATGGCCATATGAAGCCCCTCAC |
| | | GGCTTCCCAGGTCTCGGTTCACATTGGGATGGATGACAGATGGCG |
| | | GGACTCCCCACGCGCCACACCCTGGGACCAGGTATCCTATGTGAG |
| | | CCAGGTGAGCCTCAGCTCCCTGACATCAGATAAACCGTCTAGGCA |
| | | CTCAGCTGCCGCAGATGATGACGGTGACAAATGGAGCCTCCTCCT |
| | | GAACGGCCGCGACGAGGAGCAGTACATCTAA |
| 38 | miniATP7B- | ATGCCTGAACAAGAAAGACAGATCACAGCAAGAGAAGGAGCATC |
| | s1co2 | TAGGAAGATCCTGAGCAAGCTCTCCCTGCCCACAAGGGCCTGGGA |
| | | GCCTGCCATGAAGAAGTCTTTTGCCTTTGATAATGTGGGTTATGA |
| | | AGGAGGGCTGGATGGCCTGGGCCCCAGCAGCCAGGTGGCAACCA |
| | | CCCTGATTGCCATTGCAGGCATGACCTGTGCATCTTGTGTGCATAG |
| | | CATTGAAGGCATGATTAGCCAGCTGGAGGGAGTGCAGCAGATCTC |
| | | TGTCTCCCTGGCTGAGGGGACAGCCACTGTCCTGTACAATCCCTCT |
| | | GTTATTTCCCCTGAGGAGCTGAGAGCTGCCATTGAGGACATGGGA |
| | | TTTGAAGCATCTGTGGTGTCTGAGTCCTGCTCCACCAATCCCCTGG |
| | | GAAATCACTCTGCTGGCAATTCTATGGTTCAGACCACAGATGGGA |
| | | CCCCAACCTCTGTGCAGGAAGTGGCACCCCATACTGGCAGACTGC |
| | | CTGCCAATCATGCTCCTGATATCCTGGCCAAGAGCCCCCAGAGCA |
| | | CCAGGGCTGTGGCACCCCAGAAGTGTTTCCTGCAGATCAAGGGCA |
| | | TGACCTGTGCCAGCTGTGTTAGCAATATTGAAAGAAACCTCCAAA |
| | | AAGAGGCTGGAGTTCTGTCTGTTCTGGTGGCTCTGATGGCTGGGA |
| | | AAGCAGAGATCAAGTATGATCCAGAGGTGATTCAGCCACTGGAG |
| | | ATTGCCCAGTTCATCCAGGATCTGGGCTTTGAGGCAGCTGTGATG |
| | | GAGGATTATGCTGGCTCTGATGGCAACATTGAACTGACCATCACA |
| | | GGCATGACCTGTGCTTCCTGTGTGCATAATATTGAGAGCAAACTG |
| | | ACCAGAACCAATGGAATCACCTATGCCTCTGTTGCCCTGGCAACC |
| | | TCTAAGGCTCTGGTGAAGTTTGACCCTGAAATCATTGGACCTAGA |
| | | GACATCATCAAAATTATTGAAGAGATTGGTTTCCATGCCAGCCTG |
| | | GCTCAGAGAAATCCCAATGCACACCATCTGGACCATAAGATGGA |
| | | AATCAAACAGTGGAAGAAGAGCTTCCTGTGTAGCCTGGTCTTTGG |
| | | GATTCCTGTCATGGCACTGATGATTTACATGCTCATTCCCTCCAAT |
| | | GAGCCCCACCAGTCCATGGTGCTGGACCACAACATCATCCCTGGG |
| | | CTGTCTATCCTCAACCTGATCTTCTTTATTCTGTGCACATTTGTCCA |
| | | GCTCCTGGGAGGCTGGTATTTCTATGTTCAGGCCTACAAATCCCTG |
| | | AGGCATAGATCTGCCAACATGGATGTTCTGATTGTGCTGGCTACA |
| | | TCCATTGCCTATGTGTACTCCCTGGTGATCCTGGTGGTTGCTGTGG |
| | | CTGAAAAGGCTGAGAGAAGCCCTGTCACCTTCTTTGACACCCCCC |
| | | CCATGCTCTTTGTGTTTATTGCACTGGGTAGGTGGCTGGAGCATCT |
| | | GGCCAAGTCCAAGACATCTGAGGCTCTGGCCAAGCTGATGTCTCT |
| | | CCAGGCCACTGAGGCCACTGTGGTGACTCTGGGTGAAGACAATCT |
| | | GATCATTAGAGAGGAACAGGTGCCTATGGAGCTGGTGCAGAGAG |
| | | GAGATATTGTCAAGGTGGTGCCAGGAGGCAAGTTCCCAGTGGATG |
| | | GAAAAGTGCTGGAGGGCAATACCATGGCTGATGAATCCCTCATCA |
| | | CTGGGGAGGCAATGCCTGTGACCAAGAAACCTGGAAGCACTGTC |
| | | ATTGCAGGCAGCATCAATGCCCATGGGTCTGTCCTGATCAAGGCC |
| | | ACCCATGTGGGCAATGATACAACTCTGGCCCAGATTGTCAAGCTG |
| | | GTGGAAGAGGCTCAGATGTCTAAAGCTCCAATCCAGCAACTGGCT |
| | | GACAGATTCTCTGGCTACTTTGTCCCCTTTATCATCATCATGAGCA |
| | | CCCTCACCCTGGTGGTGTGGATTGTCATTGGCTTCATTGACTTTGG |
| | | GGTGGTGCAGAGATACTTCCCCAACCCTAACAAACATATCTCCCA |
| | | AACAGAGGTCATTATCAGATTTGCCTTTCAGACATCTATCACTGTG |
| | | CTGTGCATTGCCTGCCCTTGCTCTCTGGGCCTGGCCACCCCCACTG |
| | | CTGTGATGGTTGGAACAGGGGTGGCTGCACAAAATGGGATCCTGA |
| | | TCAAAGGGGGCAAACCCCTGGAAATGGCACATAAGATTAAAACA |
| | | GTTATGTTTGATAAGACAGGCACCATTACTCATGGTGTCCCTAGG |
| | | GTCATGAGAGTGCTGCTGCTGGGGGATGTGGCTACACTGCCACTG |
| | | AGGAAGGTGCTGGCTGTGGTGGGCACAGCTGAGGCCTCCTCTGAA |
| | | CACCCACTGGGAGTTGCTGTCACAAAATACTGTAAGGAGGAGCTG |
| | | GGGACAGAGACCCTGGGCTATTGTACAGACTTCCAGGCTGTGCCT |
| | | GGCTGTGGCATTGGCTGTAAGGTCAGCAATGTGGAGGGCATCCTG |
| | | GCACACTCTGAGAGACCCCTCTCTGCCCCAGCCTCCCACCTGAAT |
| | | GAAGCTGGGTCTCTGCCTGCTGAGAAGGATGCTGTCCCTCAGACC |
| | | TTCTCTGTGCTGATTGGGAACAGAGAGTGGCTGAGAAGGAATGGC |
| | | CTGACCATCAGCTCTGATGTCTCTGATGCCATGACAGATCATGAG |
| | | ATGAAAGGCCAGACAGCCATTCTGGTTGCCATTGATGGTGTGCTC |
| | | TGTGGCATGATTGCCATTGCTGATGCTGTTAAACAGGAAGCTGCA |
| | | CTGGCAGTCCACACCCTCCAGTCCATGGGAGTGGATGTGGTCCTG |
| | | ATTACTGGAGACAACAGGAAAACAGCTAGGGCCATTGCTACCCA |
| | | AGTGGGGATCAATAAGGTTTTTGCAGAAGTCCTCCCCTCCCACAA |
| | | AGTGGCCAAAGTGCAGGAGCTCCAGAACAAGGGTAAAAAGGTGG |
| | | CAATGGTGGGAGATGGTGTGAATGACAGCCCAGCTCTGGCCCAG |
| | | GCTGACATGGGAGTGGCCATTGGTACTGGAACAGATGTGGCCATT |
| | | GAAGCAGCTGATGTTGTCCTCATCAGGAATGATCTCCTGGATGTG |
| | | GTGGCCAGCATCCATCTGTCCAAGAGGACTGTGAGGAGAATCAG |
| | | GATCAACCTGGTCCTGGCCCTCATCTATAATCTGGTGGGCATCCCC |
| | | ATTGCTGCTGGGGTCTTCATGCCCATTGGCATTGTGCTGCAGCCCT |
| | | GGATGGGATCTGCTGCCATGGCTGCTTCTTCTGTCTCTGTTGTGCT |
| | | GTCCAGCCTGCAGCTGAAGTGCTATAAAAAGCCAGACCTGGAGA |
| | | GATATGAAGCCCAGGCCCATGGCCATATGAAGCCCCTCACAGCTT |
| | | CCCAGGTCTCTGTTCACATTGGGATGGATGACAGATGGAGGGACT |
| | | CCCCAAGGGCCACACCCTGGGACCAGGTGTCCTATGTGAGCCAGG |
| | | TGAGCCTCAGCTCCCTGACATCTGATAAACCCTCTAGGCACTCTG |
| | | CTGCTGCAGATGATGATGGTGACAAATGGAGCCTCCTCCTGAATG |
| | | GCAGAGATGAGGAGCAGTACATCTAA |
| 39 | miniATP7B- | ATGCCCGAACAGGAAAGACAGATTACCGCCCGCGAGGGCGCTTC |
| | s1co3 | CCGCAAAATTCTGAGCAAACTGAGCCTGCCCACCCGCGCCTGGGA |
| | | ACCTGCCATGAAGAAAAGCTTTGCTTTTGATAATGTCGGCTATGA |
| | | GGGCGGCCTGGACGGCCTGGGCCCTAGCAGCCAGGTGGCTACCA |
| | | CCCTGATTGCCATTGCCGGCATGACCTGCGCCAGCTGCGTGCATA |
| | | GCATTGAGGGCATGATTAGCCAGCTGGAGGGCGTGCAGCAGATTT |
| | | CCGTGAGCCTGGCTGAGGGCACCGCTACCGTGCTGTATAATCCCT |
| | | CCGTGATTAGCCCCGAAGAACTGCGCGCTGCCATTGAAGATATGG |
| | | GCTTTGAAGCCTCCGTGGTGTCCGAAAGCTGCAGCACCAATCCCC |
| | | TGGGCAATCATTCCGCCGGCAATAGCATGGTGCAGACCACCGACG |
| | | GCACCCCCACCTCCGTGCAGGAAGTGGCTCCCCATACCGGCCGCC |
| | | TGCCCGCCAATCATGCCCCTGATATTCTGGCTAAAAGCCCCCAGA |
| | | GCACCCGCGCCGTGGCTCCTCAGAAATGCTTTCTGCAGATTAAGG |
| | | GCATGACCTGCGCTAGCTGCGTGAGCAATATTGAACGCAATCTGC |
| | | AGAAAGAAGCCGGCGTGCTGTCCGTGCTGGTGGCCCTGATGGCCG |
| | | GCAAAGCTGAAATCAAATATGATCCCGAAGTGATTCAGCCCCTGG |
| | | AAATTGCCCAGTTCATTCAGGATCTGGGCTTTGAGGCTGCCGTGA |
| | | TGGAAGATTATGCCGGCTCCGACGGCAATATTGAACTGACCATTA |
| | | CCGGCATGACCTGCGCCAGCTGCGTGCATAATATTGAAAGCAAAC |
| | | TGACCCGCACCAACGGCATTACCTATGCTTCCGTGGCCCTGGCTA |
| | | CCAGCAAAGCCCTGGTGAAATTTGATCCTGAAATTATCGGCCCCC |
| | | GCGATATTATCAAAATTATTGAAGAAATCGGCTTTCACGCCAGCC |
| | | TGGCTCAGCGCAATCCCAATGCCCATCATCTGGATCATAAGATGG |
| | | AAATCAAACAGTGGAAGAAAAGCTTTCTGTGCAGCCTGGTGTTCG |
| | | GCATTCCTGTGATGGCCCTGATGATCTATATGCTGATTCCCAGCAA |
| | | TGAACCCCATCAGAGCATGGTGCTGGATCATAATATTATTCCCGG |
| | | CCTGAGCATTCTGAATCTGATTTTCTTTATCCTGTGCACCTTTGTG |
| | | CAGCTGCTGGGCGGCTGGTATTTCTATGTGCAGGCTTATAAAAGC |
| | | CTGCGCCACCGCTCCGCCAATATGGATGTGCTGATTGTGCTGGCC |
| | | ACCAGCATTGCTTATGTCTATAGCCTGGTCATTCTGGTGGTGGCCG |
| | | TGGCCGAGAAAGCTGAACGCAGCCCCGTGACCTTCTTTGATACCC |
| | | CTCCCATGCTGTTCGTGTTTATCGCTCTGGGCCGCTGGCTGGAACA |
| | | TCTGGCCAAAAGCAAAACCTCCGAAGCCCTGGCTAAACTGATGAG |
| | | CCTGCAGGCCACCGAAGCCACCGTGGTGACCCTGGGCGAAGATA |
| | | ATCTGATTATCCGCGAAGAACAGGTGCCTATGGAACTGGTGCAGC |
| | | GCGGCGATATTGTGAAAGTGGTGCCCGGCGGCAAATTTCCCGTGG |
| | | ACGGCAAAGTGCTGGAGGGCAATACCATGGCTGATGAATCCCTG |
| | | ATTACCGGCGAAGCTATGCCTGTGACCAAGAAACCCGGCAGCACC |
| | | GTGATTGCCGGCAGCATTAATGCTCACGGCTCCGTGCTGATCAAG |
| | | GCTACCCATGTCGGCAATGATACCACCCTGGCCCAGATTGTGAAA |
| | | CTGGTGGAAGAAGCCCAGATGAGCAAAGCCCCTATTCAGCAGCT |
| | | GGCTGACCGCTTTTCCGGCTATTTTGTGCCCTTTATCATTATTATG |
| | | AGCACCCTGACCCTGGTGGTGTGGATTGTGATCGGCTTCATTGATT |
| | | TCGGCGTGGTGCAGAGATATTTTCCTAATCCCAACAAACATATTA |
| | | GCCAGACCGAAGTGATTATCCGCTTTGCTTTTCAGACCAGCATTA |
| | | CCGTGCTGTGCATTGCTTGCCCCTGCAGCCTGGGCCTGGCTACCCC |
| | | CACCGCCGTCATGGTCGGCACCGGCGTGGCTGCCCAGAACGGCAT |
| | | TCTGATTAAGGGCGGCAAACCTCTGGAAATGGCCCACAAAATCAA |
| | | AACCGTGATGTTTGATAAAACCGGCACCATTACCCACGGCGTGCC |
| | | CCGCGTGATGCGCGTGCTGCTGCTGGGCGATGTGGCTACCCTGCC |
| | | CCTGCGCAAAGTGCTGGCCGTGGTCGGCACCGCCGAAGCTAGCTC |
| | | CGAACATCCCCTGGGCGTGGCCGTGACCAAATATTGCAAAGAAG |
| | | AACTGGGCACCGAAACCCTGGGCTATTGCACCGATTTTCAGGCCG |
| | | TCCCTGGCTGCGGCATCGGCTGCAAAGTGAGCAATGTGGAGGGCA |
| | | TTCTGGCCCATTCCGAAAGACCCCTGTCCGCCCCTGCTAGCCATCT |
| | | GAATGAAGCCGGCAGCCTGCCTGCTGAGAAAGATGCCGTGCCCC |
| | | AGACCTTTTCCGTGCTGATCGGCAACCGCGAATGGCTGCGCCGCA |
| | | ACGGCCTGACCATTAGCTCCGATGTCTCCGATGCCATGACCGATC |
| | | ATGAAATGAAAGGCCAGACCGCCATTCTGGTGGCCATTGACGGCG |
| | | TGCTGTGCGGCATGATTGCCATTGCCGATGCCGTGAAACAGGAGG |
| | | CTGCCCTGGCTGTGCATACCCTGCAGAGCATGGGCGTGGATGTGG |
| | | TGCTGATTACCGGCGATAACCGCAAAACCGCTCGCGCCATTGCCA |
| | | CCCAGGTCGGCATTAACAAAGTGTTTGCTGAAGTGCTGCCCAGCC |
| | | ATAAAGTGGCTAAAGTGCAGGAACTGCAGAACAAAGGCAAGAAA |
| | | GTGGCTATGGTCGGCGACGGCGTGAATGATAGCCCTGCCCTGGCC |
| | | CAGGCCGATATGGGCGTGGCTATCGGCACCGGCACCGATGTGGCT |
| | | ATTGAGGCTGCCGATGTGGTGCTGATCCGCAATGATCTGCTGGAT |
| | | GTGGTGGCTAGCATTCATCTGAGCAAACGCACCGTCCGCCGCATC |
| | | CGCATTAATCTGGTGCTGGCCCTGATCTATAATCTGGTCGGCATTC |
| | | CCATCGCCGCCGGCGTGTTTATGCCCATCGGCATTGTGCTGCAGC |
| | | CCTGGATGGGCAGCGCCGCTATGGCCGCTAGCTCCGTGTCCGTGG |
| | | TGCTGAGCAGCCTGCAGCTGAAATGCTATAAGAAACCCGATCTGG |
| | | AAAGATATGAAGCTCAGGCTCACGGCCATATGAAACCCCTGACCG |
| | | CCAGCCAGGTGTCCGTGCATATCGGCATGGATGACCGCTGGCGCG |
| | | ATAGCCCCCGCGCCACCCCTTGGGATCAGGTGAGCTATGTGAGCC |
| | | AGGTGTCCCTGAGCTCCCTGACCTCCGATAAACCCTCCCGCCATTC |
| | | CGCCGCTGCCGATGATGACGGCGATAAGTGGAGCCTGCTGCTGAA |
| | | CGGCCGCGATGAAGAACAGTATATCTAA |
| 40 | miniATP7B- | ATGCCTGAGCAGGAGAGACAGATCACAGCCAGAGAAGGGGCCAG |
| | s6 | TCGGAAAATCTTATCTAAGCTTTCTTTGCCTACCCGTGCCTGGGAA |
| | | CCAGCAATGAAGAAGAGTTTTGCTTTTGACAATGTTGGCTATGAA |
| | | GGTGGTCTGGATGGCCTGGGCCCTTCTTCTCAGGTGGCCCCTGAT |
| | | GGAGCCGAAGGGAGTGGGACAGATCACAGGTCTTCCAGTTCTCAT |
| | | TCCCCTGGCTCCCCACCGAGAAACCAGGTCCAGGGCACATGCAGT |
| | | ACCACTCTGATTGCCATTGCCGGCATGACCTGTGCATCCTGTGTCC |
| | | ATTCCATTGAAGGCATGATCTCCCAACTGGAAGGGGTGCAGCAAA |
| | | TATCGGTGTCTTTGGCCGAAGGGACTGCAACAGTTCTTTATAATCC |
| | | CTCTGTAATTAGCCCAGAAGAACTCAGAGCTGCTATAGAAGACAT |
| | | GGGATTTGAGGCTTCAGTCGTTTCTGAAAGCTGTTCTACTAACCCT |
| | | CTTGGAAACCACAGTGCTGGGAATTCCATGGTGCAAACTACAGAT |
| | | GGTACACCTACATCTGTGCAGGAAGTGGCTCCCCACACTGGGAGG |
| | | CTCCCTGCAAACCATGCCCCGGACATCTTGGCAAAGTCCCCACAA |
| | | TCAACCAGAGCAGTGGCACCGCAGAAGTGCTTCTTACAGATCAAA |
| | | GGCATGACCTGTGCATCCTGTGTGTCTAACATAGAAAGGAATCTG |
| | | CAGAAAGAAGCTGGTGTTCTCTCCGTGTTGGTTGCCTTGATGGCA |
| | | GGAAAGGCAGAGATCAAGTATGACCCAGAGGTCATCCAGCCCCT |
| | | CGAGATAGCTCAGTTCATCCAGGACCTGGGTTTTGAGGCAGCAGT |
| | | CATGGAGGACTACGCAGGCTCCGATGGCAACATTGAGCTGACAAT |
| | | CACAGGGATGACCTGCGCGTCCTGTGTCCACAACATAGAGTCCAA |
| | | ACTCACGAGGACAAATGGCATCACTTATGCCTCCGTTGCCCTTGC |
| | | CACCAGCAAAGCCCTTGTTAAGTTTGACCCGGAAATTATCGGTCC |
| | | ACGGGATATTATCAAAATTATTGAGGAAATTGGCTTTCATGCTTC |
| | | CCTGGCCCAGAGAAACCCCAACGCTCATCACTTGGACCACAAGAT |
| | | GGAAATAAAGCAGTGGAAGAAGTCTTTCCTGTGCAGCCTGGTGTT |
| | | TGGCATCCCTGTCATGGCCTTAATGATCTATATGCTGATACCCAGC |
| | | AACGAGCCCCACCAGTCCATGGTCCTGGACCACAACATCATTCCA |
| | | GGACTGTCCATTCTAAATCTCATCTTCTTTATCTTGTGTACCTTTGT |
| | | CCAGCTCCTCGGTGGGTGGTACTTCTACGTTCAGGCCTACAAATCT |
| | | CTGAGACACAGGTCAGCCAACATGGACGTGCTCATCGTCCTGGCC |
| | | ACAAGCATTGCTTATGTTTATTCTCTGGTCATCCTGGTGGTTGCTG |
| | | TGGCTGAGAAGGCGGAGAGGAGCCCTGTGACATTCTTCGACACGC |
| | | CCCCCATGCTCTTTGTGTTCATTGCCCTGGGCCGGTGGCTGGAACA |
| | | CTTGGCAAAGAGCAAAACCTCAGAAGCCCTGGCTAAACTCATGTC |
| | | TCTCCAAGCCACAGAAGCCACCGTTGTGACCCTTGGTGAGGACAA |
| | | TTTAATCATCAGGGAGGAGCAAGTCCCCATGGAGCTGGTGCAGCG |
| | | GGGCGATATCGTCAAGGTGGTCCCTGGGGGAAAGTTTCCAGTGGA |
| | | TGGGAAAGTCCTGGAAGGCAATACCATGGCTGATGAGTCCCTCAT |
| | | CACAGGAGAAGCCATGCCAGTCACTAAGAAACCCGGAAGCACTG |
| | | TAATTGCGGGGTCTATAAATGCACATGGCTCTGTGCTCATTAAAG |
| | | CTACCCACGTGGGCAATGACACCACTTTGGCTCAGATTGTGAAAC |
| | | TGGTGGAAGAGGCTCAGATGTCAAAGGCACCCATTCAGCAGCTG |
| | | GCTGACCGGTTTAGTGGATATTTTGTCCCATTTATCATCATCATGT |
| | | CAACTTTGACGTTGGTGGTATGGATTGTAATCGGTTTTATCGATTT |
| | | TGGTGTTGTTCAGAGATACTTTCCTAACCCCAACAAGCACATCTCC |
| | | CAGACAGAGGTGATCATCCGGTTTGCTTTCCAGACGTCCATCACG |
| | | GTGCTGTGCATTGCCTGCCCCTGCTCCCTGGGGCTGGCCACGCCC |
| | | ACGGCTGTCATGGTGGGCACCGGGGTGGCCGCGCAGAACGGCAT |
| | | CCTCATCAAGGGAGGCAAGCCCCTGGAGATGGCGCACAAGATAA |
| | | AGACTGTGATGTTTGACAAGACTGGCACCATTACCCATGGCGTCC |
| | | CCAGGGTCATGCGGGTGCTCCTGCTGGGGGATGTGGCCACACTGC |
| | | CCCTCAGGAAGGTTCTGGCTGTGGTGGGGACTGCGGAGGCCAGCA |
| | | GTGAACACCCCTTGGGCGTGGCAGTCACCAAATACTGTAAAGAGG |
| | | AACTTGGAACAGAGACCTTGGGATACTGCACGGACTTCCAGGCAG |
| | | TGCCAGGCTGTGGAATTGGGTGCAAAGTCAGCAACGTGGAAGGC |
| | | ATCCTGGCCCACAGTGAGCGCCCTTTGAGTGCACCGGCCAGTCAC |
| | | CTGAATGAGGCTGGCAGCCTTCCCGCAGAAAAAGATGCAGTCCCC |
| | | CAGACCTTCTCTGTGCTGATTGGAAACCGTGAGTGGCTGAGGCGC |
| | | AACGGTTTAACCATTTCTAGCGATGTCAGTGACGCTATGACAGAC |
| | | CACGAGATGAAAGGACAGACAGCCATCCTGGTGGCTATTGACGG |
| | | TGTGCTCTGTGGGATGATCGCAATCGCAGACGCTGTCAAGCAGGA |
| | | GGCTGCCCTGGCTGTGCACACGCTGCAGAGCATGGGTGTGGACGT |
| | | GGTTCTGATCACGGGGGACAACCGGAAGACAGCCAGAGCTATTG |
| | | CCACCCAGGTTGGCATCAACAAAGTCTTTGCAGAGGTGCTGCCTT |
| | | CGCACAAGGTGGCCAAGGTCCAGGAGCTCCAGAATAAAGGGAAG |
| | | AAAGTCGCCATGGTGGGGGATGGGGTCAATGACTCCCCGGCCTTG |
| | | GCCCAGGCAGACATGGGTGTGGCCATTGGCACCGGCACGGATGT |
| | | GGCCATCGAGGCAGCCGACGTCGTCCTTATCAGAAATGATTTGCT |
| | | GGATGTGGTGGCTAGCATTCACCTTTCCAAGAGGACTGTCCGAAG |
| | | GATACGCATCAACCTGGTCCTGGCACTGATTTATAACCTGGTTGG |
| | | GATACCCATTGCAGCAGGTGTCTTCATGCCCATCGGCATTGTGCT |
| | | GCAGCCCTGGATGGGCTCAGCGGCCATGGCAGCCTCCTCTGTGTC |
| | | TGTGGTGCTCTCATCCCTGCAGCTCAAGTGCTATAAGAAGCCTGA |
| | | CCTGGAGAGGTATGAGGCACAGGCGCATGGCCACATGAAGCCCC |
| | | TGACGGCATCCCAGGTCAGTGTGCACATAGGCATGGATGACAGGT |
| | | GGCGGGACTCCCCCAGGGCCACACCATGGGACCAGGTCAGCTAT |
| | | GTCAGCCAGGTGTCGCTGTCCTCCCTGACGTCCGACAAGCCATCT |
| | | CGGCACAGCGCTGCAGCAGACGATGATGGGGACAAGTGGTCTCT |
| | | GCTCCTGAATGGCAGGGATGAGGAGCAGTACATCTGA |
| 41 | miniATP7Bv. | ATGCCGGAACAAGAACGCCAGATCACAGCAAGAGAAGGAGCAAG |
| | co | TAGGAAGATCCTGAGCAAGCTCTCCCTGCCCACAAGGGCCTGGGA |
| | | GCCGGCCATGAAGAAGTCTTTTGCCTTCGATAATGTCGGTTATGA |
| | | AGGCGGGCTCGATGGCCTGGGCCCCAGCAGCCAGCCCCAGAAGT |
| | | GTTTCCTGCAGATCAAGGGCATGACCTGTGCCAGCTGTGTTAGCA |
| | | ATATCGAAAGAAACCTCCAAAAAGAGGCTGGAGTTCTGAGTGTTC |
| | | TAGTGGCTTTAATGGCCGGGAAAGCCGAGATCAAGTATGATCCCG |
| | | AGGTGATTCAGCCATTGGAGATTGCCCAGTTCATCCAGGATCTGG |
| | | GCTTCGAGGCCGCCGTGATGGAGGATTACGCCGGCTCTGATGGCA |
| | | ACATCGAACTGACCATCACCGGCATGACCTGTGCTTCCTGCGTGC |
| | | ATAATATCGAGAGCAAACTGACGCGGACCAATGGAATCACCTAT |
| | | GCCAGCGTCGCCCTGGCAACCTCTAAGGCTCTGGTGAAGTTTGAC |
| | | CCCGAAATCATTGGACCTAGAGACATCATCAAAATTATTGAAGAG |
| | | ATTGGTTTCCACGCCAGCTTAGCTCAGCGGAATCCCAACGCACAC |
| | | CATCTTGACCATAAGATGGAAATCAAACAGTGGAAGAAGAGCTT |
| | | CTTGTGTAGCCTGGTCTTCGGGATACCCGTCATGGCACTGATGATT |
| | | TACATGCTCATTCCGTCCAACGAGCCCCACCAGTCCATGGTGCTG |
| | | GACCACAACATCATCCCTGGGCTGTCTATCCTCAACCTGATCTTCT |
| | | TTATTCTGTGCACATTTGTCCAGCTCTTGGGAGGCTGGTATTTCTA |
| | | TGTACAGGCCTACAAATCCCTGAGGCATCGAAGCGCCAACATGGA |
| | | CGTTTTAATTGTGCTGGCTACATCCATTGCCTACGTGTACTCCCTG |
| | | GTGATCTTAGTGGTTGCTGTGGCTGAAAAGGCTGAGAGAAGCCCT |
| | | GTCACCTTCTTCGACACCCCCCCCATGCTCTTCGTGTTTATTGCAC |
| | | TGGGTCGCTGGCTGGAGCATTTAGCCAAGTCCAAGACATCCGAGG |
| | | CTCTGGCCAAGCTGATGTCTCTACAGGCCACGGAGGCCACCGTGG |
| | | TGACTCTGGGTGAAGACAATTTGATAATACGCGAGGAACAGGTGC |
| | | CTATGGAGCTTGTGCAGCGCGGAGATATCGTCAAGGTGGTGCCCG |
| | | GCGGCAAGTTCCCAGTGGATGGAAAAGTGTTAGAGGGCAATACC |
| | | ATGGCCGATGAATCCCTCATCACTGGCGAGGCAATGCCGGTGACC |
| | | AAGAAACCTGGAAGTACTGTCATTGCAGGCAGCATCAATGCCCAT |
| | | GGGAGCGTCCTGATCAAGGCCACCCACGTGGGCAATGATACAACT |
| | | CTCGCCCAGATCGTCAAGCTAGTGGAAGAGGCTCAGATGTCTAAA |
| | | GCTCCAATCCAGCAACTGGCTGACCGCTTCAGCGGCTACTTTGTC |
| | | CCCTTTATCATCATCATGAGCACCCTCACCCTGGTGGTGTGGATAG |
| | | TCATAGGCTTCATTGACTTTGGCGTGGTGCAGAGATACTTCCCCA |
| | | ACCCTAACAAACATATCTCCCAAACCGAGGTCATTATCAGATTCG |
| | | CCTTTCAGACATCTATCACTGTGCTGTGCATAGCCTGCCCTTGCTC |
| | | TCTGGGCCTGGCCACCCCCACCGCCGTGATGGTTGGAACCGGGGT |
| | | CGCTGCACAAAATGGGATCCTGATAAAAGGGGGCAAACCCCTGG |
| | | AAATGGCACATAAGATTAAAACAGTTATGTTTGATAAGACAGGCA |
| | | CCATTACTCACGGTGTCCCTCGAGTCATGAGAGTGCTGCTGCTGG |
| | | GGGACGTGGCTACACTGCCACTGCGCAAGGTGCTGGCTGTGGTGG |
| | | GCACAGCGGAGGCCTCCTCAGAACACCCATTAGGCGTAGCTGTCA |
| | | CAAAATACTGTAAGGAGGAGCTGGGGACAGAGACCCTCGGCTAT |
| | | TGTACAGACTTCCAGGCCGTGCCTGGCTGCGGCATCGGCTGTAAG |
| | | GTCAGCAATGTGGAGGGCATCCTGGCACACAGCGAGAGACCCCT |
| | | CTCTGCCCCAGCCTCCCACCTGAATGAAGCTGGGTCTCTGCCTGCT |
| | | GAGAAGGACGCCGTCCCTCAGACCTTCTCCGTGTTGATTGGGAAC |
| | | AGAGAGTGGCTTAGAAGGAATGGCCTGACCATCAGCTCTGACGTC |
| | | TCGGATGCCATGACAGATCACGAGATGAAAGGCCAGACAGCCAT |
| | | ACTGGTTGCCATCGACGGCGTGCTCTGTGGCATGATTGCCATCGC |
| | | TGATGCTGTTAAACAGGAAGCTGCGCTGGCAGTCCACACCCTCCA |
| | | GTCCATGGGAGTGGACGTGGTCCTGATTACTGGAGACAACAGGA |
| | | AAACCGCTCGGGCCATCGCTACCCAAGTGGGGATAAATAAGGTTT |
| | | TTGCAGAAGTCTTACCCTCCCACAAAGTAGCCAAAGTGCAGGAGC |
| | | TCCAGAACAAGGGTAAAAAGGTGGCAATGGTGGGAGATGGTGTG |
| | | AATGACAGCCCAGCTCTCGCGCAGGCTGACATGGGCGTAGCCATA |
| | | GGTACTGGAACAGATGTCGCGATTGAAGCCGCTGACGTAGTCCTC |
| | | ATCCGAAACGATTTGCTGGATGTGGTGGCCAGCATCCATCTGTCC |
| | | AAGCGTACCGTGCGCCGGATCCGCATCAACCTGGTCTTGGCCCTC |
| | | ATCTATAATCTCGTGGGCATCCCCATCGCTGCTGGGGTCTTCATGC |
| | | CCATCGGCATTGTGCTGCAGCCCTGGATGGGAAGCGCTGCCATGG |
| | | CTGCTTCTTCTGTCTCCGTTGTGCTGTCCAGCCTACAGCTGAAGTG |
| | | CTATAAAAAGCCAGACCTCGAGCGCTACGAAGCCCAGGCGCATG |
| | | GCCATATGAAGCCCCTCACGGCTTCCCAGGTCTCGGTTCACATTG |
| | | GGATGGATGACAGATGGCGGGACTCCCCACGCGCCACACCCTGG |
| | | GACCAGGTATCCTATGTGAGCCAGGTGAGCCTCAGCTCCCTGACA |
| | | TCAGATAAACCGTCTAGGCACTCAGCTGCCGCAGATGATGACGGT |
| | | GACAAATGGAGCCTCCTCCTGAACGGCCGCGACGAGGAGCAGTA |
| | | CATCTAA |
| 42 | miniATP7Bv_ | ATGCCTGAACAGGAGAGACAGATCACAGCCAGAGAAGGGGCCAG |
| | v2 | TCGGAAAATCTTATCTAAGCTTTCTTTGCCTACCCGTGCCTGGGAA |
| | | CCAGCAATGAAGAAGAGTTTTGCTTTTGACAATGTTGGCTATGAA |
| | | GGTGGTCTGGATGGCCTGGGCCCTTCTTCTCAGCCGCAGAAGTGC |
| | | TTCTTACAGATCAAAGGCATGACCTGTGCATCCTGTGTGTCTAAC |
| | | ATAGAAAGGAATCTGCAGAAAGAAGCTGGTGTTCTCTCCGTGTTG |
| | | GTTGCCTTGATGGCAGGAAAGGCAGAGATCAAGTATGACCCAGA |
| | | GGTCATCCAGCCCCTCGAGATAGCTCAGTTCATCCAGGACCTGGG |
| | | TTTTGAGGCAGCAGTCATGGAGGACTACGCAGGCTCCGATGGCAA |
| | | CATTGAGCTGACAATCACAGGGATGACCTGCGCGTCCTGTGTCCA |
| | | CAACATAGAGTCCAAACTCACGAGGACAAATGGCATCACTTATGC |
| | | CTCCGTTGCCCTTGCCACCAGCAAAGCCCTTGTTAAGTTTGACCCG |
| | | GAAATTATCGGTCCACGGGATATTATCAAAATTATTGAGGAAATT |
| | | GGCTTTCATGCTTCCCTGGCCCAGAGAAACCCCAACGCTCATCAC |
| | | TTGGACCACAAGATGGAAATAAAGCAGTGGAAGAAGTCTTTCCTG |
| | | TGCAGCCTGGTGTTTGGCATCCCTGTCATGGCCTTAATGATCTATA |
| | | TGCTGATACCCAGCAACGAGCCCCACCAGTCCATGGTCCTGGACC |
| | | ACAACATCATTCCAGGACTGTCCATTCTAAATCTCATCTTCTTTAT |
| | | CTTGTGTACCTTTGTCCAGCTCCTCGGTGGGTGGTACTTCTACGTT |
| | | CAGGCCTACAAATCTCTGAGACACAGGTCAGCCAACATGGACGTG |
| | | CTCATCGTCCTGGCCACAAGCATTGCTTATGTTTATTCTCTGGTCA |
| | | TCCTGGTGGTTGCTGTGGCTGAGAAGGCGGAGAGGAGCCCTGTGA |
| | | CATTCTTCGACACGCCCCCCATGCTCTTTGTGTTCATTGCCCTGGG |
| | | CCGGTGGCTGGAACACTTGGCAAAGAGCAAAACCTCAGAAGCCC |
| | | TGGCTAAACTCATGTCTCTCCAAGCCACAGAAGCCACCGTTGTGA |
| | | CCCTTGGTGAGGACAATTTAATCATCAGGGAGGAGCAAGTCCCCA |
| | | TGGAGCTGGTGCAGCGGGGCGATATCGTCAGGGTGGTCCCTGGGG |
| | | GAAAGTTTCCAGTGGATGGGAAAGTCCTGGAAGGCAATACCATG |
| | | GCTGATGAGTCCCTCATCACAGGAGAAGCCATGCCAGTCACTAAG |
| | | AAACCCGGAAGCACTGTAATTGCGGGGTCTATAAATGCACATGGC |
| | | TCTGTGCTCATTAAAGCTACCCACGTGGGCAATGACACCACTTTG |
| | | GCTCAGATTGTGAAACTGGTGGAAGAGGCTCAGATGTCAAAGGC |
| | | ACCCATTCAGCAGCTGGCTGACCGGTTTAGTGGATATTTTGTCCCA |
| | | TTTATCATCATCATGTCAACTTTGACGTTGGTGGTATGGATTGTAA |
| | | TCGGTTTTATCGATTTTGGTGTTGTTCAGAAATACTTTCCTAACCC |
| | | CAACAAGCACATCTCCCAGACAGAGGTGATCATCCGGTTTGCTTT |
| | | CCAGACGTCCATCACGGTGCTGTGCATTGCCTGCCCCTGCTCCCTG |
| | | GGGCTGGCCACGCCCACGGCTGTCATGGTGGGCACCGGGGTGGCC |
| | | GCGCAGAACGGCATCCTCATCAAGGGAGGCAAGCCCCTGGAGAT |
| | | GGCGCACAAGATAAAGACTGTGATGTTTGACAAGACTGGCACCAT |
| | | TACCCATGGCGTCCCCAGGGTCATGCGGGTGCTCCTGCTGGGGGA |
| | | TGTGGCCACACTGCCCCTCAGGAAGGTTCTGGCTGTGGTGGGGAC |
| | | TGCGGAGGCCAGCAGTGAACACCCCTTGGGCGTGGCAGTCACCA |
| | | AATACTGTAAAGAGGAACTTGGAACAGAGACCTTGGGATACTGC |
| | | ACGGACTTCCAGGCAGTGCCAGGCTGTGGAATTGGGTGCAAAGTC |
| | | AGCAACGTGGAAGGCATCCTGGCCCACAGTGAGCGCCCTTTGAGT |
| | | GCACCGGCCAGTCACCTGAATGAGGCTGGCAGCCTTCCCGCAGAA |
| | | AAAGATGCAGCCCCCCAGACCTTCTCTGTGCTGATTGGAAACCGT |
| | | GAGTGGCTGAGGCGCAACGGTTTAACCATTTCTAGCGATGTCAGT |
| | | GACGCTATGACAGACCACGAGATGAAAGGACAGACAGCCATCCT |
| | | GGTGGCTATTGACGGTGTGCTCTGTGGGATGATCGCAATCGCAGA |
| | | CGCTGTCAAGCAGGAGGCTGCCCTGGCTGTGCACACGCTGCAGAG |
| | | CATGGGTGTGGACGTGGTTCTGATCACGGGGGACAACCGGAAGA |
| | | CAGCCAGAGCTATTGCCACCCAGGTTGGCATCAACAAAGTCTTTG |
| | | CAGAGGTGCTGCCTTCGCACAAGGTGGCCAAGGTCCAGGAGCTCC |
| | | AGAATAAAGGGAAGAAAGTCGCCATGGTGGGGGATGGGGTCAAT |
| | | GACTCCCCGGCCTTGGCCCAGGCAGACATGGGTGTGGCCATTGGC |
| | | ACCGGCACGGATGTGGCCATCGAGGCAGCCGACGTCGTCCTTATC |
| | | AGAAATGATTTGCTGGATGTGGTGGCTAGCATTCACCTTTCCAAG |
| | | AGGACTGTCCGAAGGATACGCATCAACCTGGTCCTGGCACTGATT |
| | | TATAACCTGGTTGGGATACCCATTGCAGCAGGTGTCTTCATGCCC |
| | | ATCGGCATTGTGCTGCAGCCCTGGATGGGCTCAGCGGCCATGGCA |
| | | GCCTCCTCTGTGTCTGTGGTGCTCTCATCCCTGCAGCTCAAGTGCT |
| | | ATAAGAAGCCTGACCTGGAGAGGTATGAGGCACAGGCGCATGGC |
| | | CACATGAAGCCCCTGACGGCATCCCAGGTCAGTGTGCACATAGGC |
| | | ATGGATGACAGGTGGCGGGACTCCCCCAGGGCCACACCATGGGA |
| | | CCAGGTCAGCTATGTCAGCCAGGTGTCGCTGTCCTCCCTGACGTC |
| | | CGACAAGCCATCTCGGCACAGCGCTGCAGCAGACGATGATGGGG |
| | | ACAAGTGGTCTCTGCTCCTGAATGGCAGGGATGAGGAGCAGTACA |
| | | TCTGA |
| 43 | miniATP7B- | ATGCCTGAGCAGGAGAGACAGATCACAGCCAGAGAAGGGGCCAG |
| | WD4 | TCGGAAAATCTTATCTAAGCTTTCTTTGCCTACCCGTGCCTGGGAA |
| | | CCAGCAATGAAGAAGAGTTTTGCTTTTGACAATGTTGGCTATGAA |
| | | GGTGGTCTGGATGGCCTGGGCCCTTCTTCTCAGGTGGCCACCAGC |
| | | ACAGTCAGGATCTTGGGCATGACTTGCCAGTCATGTGTGAAGTCC |
| | | ATTGAGGACAGGATTTCCAATTTGAAAGGCATCATCAGCATGAAG |
| | | GTTTCCCTGGAACAAGGCAGTGCCACTGTGAAATATGTGCCATCG |
| | | GTTGTGTGCCTGCAACAGGTTTGCCATCAAATTGGGGACATGGGC |
| | | TTCGAGGCCAGCATTCCTGATGGAGCCGAAGGGAGTGGGACAGA |
| | | TCACAGGTCTTCCAGTTCTCATTCCCCTGGCTCCCCACCGAGAAAC |
| | | CAGGTCCAGGGCACATGCAGTAAGTGCTTCTTACAGATCAAAGGC |
| | | ATGACCTGTGCATCCTGTGTGTCTAACATAGAAAGGAATCTGCAG |
| | | AAAGAAGCTGGTGTTCTCTCCGTGTTGGTTGCCTTGATGGCAGGA |
| | | AAGGCAGAGATCAAGTATGACCCAGAGGTCATCCAGCCCCTCGA |
| | | GATAGCTCAGTTCATCCAGGACCTGGGTTTTGAGGCAGCAGTCAT |
| | | GGAGGACTACGCAGGCTCCGATGGCAACATTGAGCTGACAATCA |
| | | CAGGGATGACCTGCGCGTCCTGTGTCCACAACATAGAGTCCAAAC |
| | | TCACGAGGACAAATGGCATCACTTATGCCTCCGTTGCCCTTGCCA |
| | | CCAGCAAAGCCCTTGTTAAGTTTGACCCGGAAATTATCGGTCCAC |
| | | GGGATATTATCAAAATTATTGAGGAAATTGGCTTTCATGCTTCCCT |
| | | GGCCCAGAGAAACCCCAACGCTCATCACTTGGACCACAAGATGG |
| | | AAATAAAGCAGTGGAAGAAGTCTTTTCTTGTGAGTCTGTTTTTCTG |
| | | TATTCCTGTAATGGGGCTGATGATATATATGATGGTTATGGACCA |
| | | CCACTTTGCAACTCTTCACCATAATCAAAACATGAGTAAAGAAGA |
| | | AATGATCAACCTTCATTCTTCTATGTTCCTGGAGCGCCAGATTCTT |
| | | CCAGGATTGTCTGTTATGAATTTGCTGTCCTTTTTATTGTGTGTAC |
| | | CTGTACAGTTTTTCGGAGGCTGGTACTTCTACATTCAGGCTTATAA |
| | | AGCACTGAAGCATAAGACAGCAAATATGGACGTACTGATTGTGCT |
| | | GGCAACCACCATTGCATTTGCCTACTCTTTGATTATTCTTCTAGTT |
| | | GCAATGTATGAGAGAGCCAAAGTGAACCCTATTACTTTCTTTGAC |
| | | ACACCCCCTATGCTGTTTGTGTTTATTGCACTAGGCCGATGGCTGG |
| | | AACATATAGCAAAGGGCAAAACATCAGAGGCTCTTGCAAAGTTA |
| | | ATTTCACTACAAGCTACAGAAGCAACTATTGTAACTCTTGATTCTG |
| | | ATAATATCCTCCTCAGTGAAGAACAAGTGGATGTGGAACTTGTAC |
| | | AACGTGGAGATATCATTAAAGTAGTTCCAGGAGGCAAATTTCCAG |
| | | TGGATGGTCGTGTTATTGAAGGACATTCTATGGTAGATGAGTCCC |
| | | TCATCACAGGGGAGGCAATGCCTGTGGCTAAGAAACCTGGCAGC |
| | | ACAGTGATTGCTGGTTCCATTAACCAGAACGGGTCACTGCTTATC |
| | | TGCGCAACACATGTTGGAGCAGACACAACCCTTTCTCAAATTGTC |
| | | AAACTTGTGGAAGAGGCACAAACATCAAAGGCTCCTATCCAGCA |
| | | GTTTGCAGACAAACTCAGTGGCTATTTTGTTCCTTTTATTGTTTTTG |
| | | TTTCCATTGCCACCCTCTTGGTATGGATTGTAATTGGATTTCTGAA |
| | | TTTTGAAATTGTGGAAACCTACTTTCCTGGCTACAATAGAAGTATC |
| | | TCCCGAACAGAAACGATAATACGATTTGCTTTCCAAGCCTCTATC |
| | | ACAGTTCTGTGTATTGCATGTCCCTGTTCACTGGGACTGGCCACTC |
| | | CAACTGCTGTGATGGTGGGTACAGGAGTAGGTGCTCAAAATGGCA |
| | | TACTAATAAAAGGTGGAGAGCCATTGGAGATGGCTCATAAGGTA |
| | | AAGGTAGTGGTATTTGATAAGACTGGAACCATTACTCACGGAACC |
| | | CCAGTGGTGAATCAAGTAAAGGTTCTAACTGAAAGTAACAGAAT |
| | | ATCACACCATAAAATCTTGGCCATTGTGGGAACTGCTGAAAGTAA |
| | | CAGTGAACACCCTCTAGGAACAGCCATAACCAAATATTGCAAACA |
| | | GGAGCTGGACACTGAAACCTTGGGTACCTGCATAGATTTCCAGGT |
| | | TGTGCCAGGCTGTGGTATTAGCTGTAAAGTCACCAATATTGAAGG |
| | | CTTGCTACATAAGAATAACTGGAATATAGAGGACAATAATATTAA |
| | | AAATGCATCCCTGGTTCAAATTGATGCCAGTAATGAACAGTCATC |
| | | AACTTCGTCTTCCATGATTATTGATGCCCAGATCTCAAATGCTCTT |
| | | AATGCTCAGCAGTATAAAGTCCTCATTGGTAACCGGGAGTGGATG |
| | | ATTAGAAATGGTCTTGTCATTAATAACGATGTAAATGATTTCATG |
| | | ACTGAACATGAGAGAAAAGGTCGGACTGCTGTATTAGTAGCAGTT |
| | | GATGATGAGCTGTGTGGCTTGATAGCCATTGCAGACACAGTGAAG |
| | | CCTGAAGCAGAACTGGCTATCCATATTCTGAAATCTATGGGCTTA |
| | | GAAGTAGTTCTGATGACTGGAGACAACAGTAAAACAGCTAGATCT |
| | | ATTGCTTCTCAGGTTGGCATTACTAAGGTGTTTGCTGAAGTTCTAC |
| | | CTTCTCACAAGGTTGCTAAAGTGAAGCAACTTCAAGAGGAGGGG |
| | | AAACGGGTAGCAATGGTGGGAGATGGAATCAATGACTCCCCAGC |
| | | TCTGGCAATGGCTAATGTGGGAATTGCTATTGGCACAGGCACAGA |
| | | TGTAGCCATTGAAGCAGCTGATGTGGTTTTGATAAGGAATGATCT |
| | | TCTGGATGTAGTGGCAAGTATTGACTTATCAAGAAAGACAGTCAA |
| | | GAGGATTCGGATAAATTTTGTCTTTGCTCTAATTTATAATCTGGTT |
| | | GGAATTCCCATAGCTGCTGGAGTTTTTATGCCCATTGGTTTGGTTT |
| | | TGCAGCCCTGGATGGGATCTGCAGCAATGGCTGCTTCATCTGTTTC |
| | | TGTAGTACTTTCTTCTCTCCAGCTCAAGTGCTATAAGAAGCCTGAC |
| | | CTGGAGAGGTATGAGGCACAGGCGCATGGCCACATGAAGCCCCT |
| | | GACGGCATCCCAGGTCAGTGTGCACATAGGCATGGATGACAGGT |
| | | GGCGGGACTCCCCCAGGGCCACACCATGGGACCAGGTCAGCTAT |
| | | GTCAGCCAGGTGTCGCTGTCCTCCCTGACGTCCGACAAGCCATCT |
| | | CGGCACAGCGCTGCAGCAGACGATGATGGGGACAAGTGGTCTCT |
| | | GCTCCTGAATGGCAGGGATGAGGAGCAGTACATCTGA |
| 44 | miniATP7B- | ATGCCTGAGCAGGAGAGACAGATCACAGCCAGAGAAGGGGCCAG |
| | WD5 | TCGGAAAATCTTATCTAAGCTTTCTTTGCCTACCCGTGCCTGGGAA |
| | | CCAGCAATGAAGAAGAGTTTTGCTTTTGACAATGTTGGCTATGAA |
| | | GGTGGTCTGGATGGCCTGGGCCCTTCTTCTCAGGTGGCCCCTGAT |
| | | GGAGCCGAAGGGAGTGGGACAGATCACAGGTCTTCCAGTTCTCAT |
| | | TCCCCTGGCTCCCCACCGAGAAACCAGGTCCAGGGCACATGCAGT |
| | | ACCACTCTGATTGCCATTGCCGGCATGACCTGTGCATCCTGTGTCC |
| | | ATTCCATTGAAGGCATGATCTCCCAACTGGAAGGGGTGCAGCAAA |
| | | TATCGGTGTCTTTGGCCGAAGGGACTGCAACAGTTCTTTATAATCC |
| | | CTCTGTAATTAGCCCAGAAGAACTCAGAGCTGCTATAGAAGACAT |
| | | GGGATTTGAGGCTTCAGTCGTTTCTGAAAGCTGTTCTACTAACCCT |
| | | CTTGGAAACCACAGTGCTGGGAATTCCATGGTGCAAACTACAGAT |
| | | GGTACACCTACATCTGTGCAGGAAGTGGCTCCCCACACTGGGAGG |
| | | CTCCCTGCAAACCATGCCCCGGACATCTTGGCAAAGTCCCCACAA |
| | | TCAACCAGAGCAGTGGCACCGCAGAAGTGCTTCTTACAGATCAAA |
| | | GGCATGACCTGTGCATCCTGTGTGTCTAACATAGAAAGGAATCTG |
| | | CAGAAAGAAGCTGGTGTTCTCTCCGTGTTGGTTGCCTTGATGGCA |
| | | GGAAAGGCAGAGATCAAGTATGACCCAGAGGTCATCCAGCCCCT |
| | | CGAGATAGCTCAGTTCATCCAGGACCTGGGTTTTGAGGCAGCAGT |
| | | CATGGAGGACTACGCAGGCTCCGATGGCAACATTGAGCTGACAAT |
| | | CACAGGGATGACCTGCGCGTCCTGTGTCCACAACATAGAGTCCAA |
| | | ACTCACGAGGACAAATGGCATCACTTATGCCTCCGTTGCCCTTGC |
| | | CACCAGCAAAGCCCTTGTTAAGTTTGACCCGGAAATTATCGGTCC |
| | | ACGGGATATTATCAAAATTATTGAGGAAATTGGCTTTCATGCTTC |
| | | CCTGGCCCAGAGAAACCCCAACGCTCATCACTTGGACCACAAGAT |
| | | GGAAATAAAGCAGTGGAAGAAGTCTTTTCTTGTGAGTCTGTTTTT |
| | | CTGTATTCCTGTAATGGGGCTGATGATATATATGATGGTTATGGA |
| | | CCACCACTTTGCAACTCTTCACCATAATCAAAACATGAGTAAAGA |
| | | AGAAATGATCAACCTTCATTCTTCTATGTTCCTGGAGCGCCAGATT |
| | | CTTCCAGGATTGTCTGTTATGAATTTGCTGTCCTTTTTATTGTGTGT |
| | | ACCTGTACAGTTTTTCGGAGGCTGGTACTTCTACATTCAGGCTTAT |
| | | AAAGCACTGAAGCATAAGACAGCAAATATGGACGTACTGATTGT |
| | | GCTGGCAACCACCATTGCATTTGCCTACTCTTTGATTATTCTTCTA |
| | | GTTGCAATGTATGAGAGAGCCAAAGTGAACCCTATTACTTTCTTT |
| | | GACACACCCCCTATGCTGTTTGTGTTTATTGCACTAGGCCGATGGC |
| | | TGGAACATATAGCAAAGGGCAAAACATCAGAGGCTCTTGCAAAG |
| | | TTAATTTCACTACAAGCTACAGAAGCAACTATTGTAACTCTTGATT |
| | | CTGATAATATCCTCCTCAGTGAAGAACAAGTGGATGTGGAACTTG |
| | | TACAACGTGGAGATATCATTAAAGTAGTTCCAGGAGGCAAATTTC |
| | | CAGTGGATGGTCGTGTTATTGAAGGACATTCTATGGTAGATGAGT |
| | | CCCTCATCACAGGGGAGGCAATGCCTGTGGCTAAGAAACCTGGCA |
| | | GCACAGTGATTGCTGGTTCCATTAACCAGAACGGGTCACTGCTTA |
| | | TCTGCGCAACACATGTTGGAGCAGACACAACCCTTTCTCAAATTG |
| | | TCAAACTTGTGGAAGAGGCACAAACATCAAAGGCTCCTATCCAGC |
| | | AGTTTGCAGACAAACTCAGTGGCTATTTTGTTCCTTTTATTGTTTTT |
| | | GTTTCCATTGCCACCCTCTTGGTATGGATTGTAATTGGATTTCTGA |
| | | ATTTTGAAATTGTGGAAACCTACTTTCCTGGCTACAATAGAAGTA |
| | | TCTCCCGAACAGAAACGATAATACGATTTGCTTTCCAAGCCTCTA |
| | | TCACAGTTCTGTGTATTGCATGTCCCTGTTCACTGGGACTGGCCAC |
| | | TCCAACTGCTGTGATGGTGGGTACAGGAGTAGGTGCTCAAAATGG |
| | | CATACTAATAAAAGGTGGAGAGCCATTGGAGATGGCTCATAAGG |
| | | TAAAGGTAGTGGTATTTGATAAGACTGGAACCATTACTCACGGAA |
| | | CCCCAGTGGTGAATCAAGTAAAGGTTCTAACTGAAAGTAACAGA |
| | | ATATCACACCATAAAATCTTGGCCATTGTGGGAACTGCTGAAAGT |
| | | AACAGTGAACACCCTCTAGGAACAGCCATAACCAAATATTGCAA |
| | | ACAGGAGCTGGACACTGAAACCTTGGGTACCTGCATAGATTTCCA |
| | | GGTTGTGCCAGGCTGTGGTATTAGCTGTAAAGTCACCAATATTGA |
| | | AGGCTTGCTACATAAGAATAACTGGAATATAGAGGACAATAATAT |
| | | TAAAAATGCATCCCTGGTTCAAATTGATGCCAGTAATGAACAGTC |
| | | ATCAACTTCGTCTTCCATGATTATTGATGCCCAGATCTCAAATGCT |
| | | CTTAATGCTCAGCAGTATAAAGTCCTCATTGGTAACCGGGAGTGG |
| | | ATGATTAGAAATGGTCTTGTCATTAATAACGATGTAAATGATTTC |
| | | ATGACTGAACATGAGAGAAAAGGTCGGACTGCTGTATTAGTAGC |
| | | AGTTGATGATGAGCTGTGTGGCTTGATAGCCATTGCAGACACAGT |
| | | GAAGCCTGAAGCAGAACTGGCTATCCATATTCTGAAATCTATGGG |
| | | CTTAGAAGTAGTTCTGATGACTGGAGACAACAGTAAAACAGCTAG |
| | | ATCTATTGCTTCTCAGGTTGGCATTACTAAGGTGTTTGCTGAAGTT |
| | | CTACCTTCTCACAAGGTTGCTAAAGTGAAGCAACTTCAAGAGGAG |
| | | GGGAAACGGGTAGCAATGGTGGGAGATGGAATCAATGACTCCCC |
| | | AGCTCTGGCAATGGCTAATGTGGGAATTGCTATTGGCACAGGCAC |
| | | AGATGTAGCCATTGAAGCAGCTGATGTGGTTTTGATAAGGAATGA |
| | | TCTTCTGGATGTAGTGGCAAGTATTGACTTATCAAGAAAGACAGT |
| | | CAAGAGGATTCGGATAAATTTTGTCTTTGCTCTAATTTATAATCTG |
| | | GTTGGAATTCCCATAGCTGCTGGAGTTTTTATGCCCATTGGTTTGG |
| | | TTTTGCAGCCCTGGATGGGATCTGCAGCAATGGCTGCTTCATCTGT |
| | | TTCTGTAGTACTTTCTTCTCTCCAGCTCAAGTGCTATAAGAAGCCT |
| | | GACCTGGAGAGGTATGAGGCACAGGCGCATGGCCACATGAAGCC |
| | | CCTGACGGCATCCCAGGTCAGTGTGCACATAGGCATGGATGACAG |
| | | GTGGCGGGACTCCCCCAGGGCCACACCATGGGACCAGGTCAGCT |
| | | ATGTCAGCCAGGTGTCGCTGTCCTCCCTGACGTCCGACAAGCCAT |
| | | CTCGGCACAGCGCTGCAGCAGACGATGATGGGGACAAGTGGTCT |
| | | CTGCTCCTGAATGGCAGGGATGAGGAGCAGTACATCTGA |
| 45 | miniATP7B- | ATGCCTGAGCAGGAGAGACAGATCACAGCCAGAGAAGGGGCCAG |
| | WD6 | TCGGAAAATCTTATCTAAGCTTTCTTTGCCTACCCGTGCCTGGGAA |
| | | CCAGCAATGAAGAAGAGTTTTGCTTTTGACAATGTTGGCTATGAA |
| | | GGTGGTCTGGATGGCCTGGGCCCTTCTTCTCAGGTGGCCCCTGAT |
| | | GGAGCCGAAGGGAGTGGGACAGATCACAGGTCTTCCAGTTCTCAT |
| | | TCCCCTGGCTCCCCACCGAGAAACCAGGTCCAGGGCACATGCAGT |
| | | AAGTGCTTCTTACAGATCAAAGGCATGACCTGTGCATCCTGTGTG |
| | | TCTAACATAGAAAGGAATCTGCAGAAAGAAGCTGGTGTTCTCTCC |
| | | GTGTTGGTTGCCTTGATGGCAGGAAAGGCAGAGATCAAGTATGAC |
| | | CCAGAGGTCATCCAGCCCCTCGAGATAGCTCAGTTCATCCAGGAC |
| | | CTGGGTTTTGAGGCAGCAGTCATGGAGGACTACGCAGGCTCCGAT |
| | | GGCAACATTGAGCTGACAATCACAGGGATGACCTGCGCGTCCTGT |
| | | GTCCACAACATAGAGTCCAAACTCACGAGGACAAATGGCATCACT |
| | | TATGCCTCCGTTGCCCTTGCCACCAGCAAAGCCCTTGTTAAGTTTG |
| | | ACCCGGAAATTATCGGTCCACGGGATATTATCAAAATTATTGAGG |
| | | AAATTGGCTTTCATGCTTCCCTGGCCCAGAGAAACCCCAACGCTC |
| | | ATCACTTGGACCACAAGATGGAAATAAAGCAGTGGAAGAAGTCT |
| | | TTTCTTGTGAGTCTGTTTTTCTGTATTCCTGTAATGGGGCTGATGA |
| | | TATATATGATGGTTATGGACCACCACTTTGCAACTCTTCACCATAA |
| | | TCAAAACATGAGTAAAGAAGAAATGATCAACCTTCATTCTTCTAT |
| | | GTTCCTGGAGCGCCAGATTCTTCCAGGATTGTCTGTTATGAATTTG |
| | | CTGTCCTTTTTATTGTGTGTACCTGTACAGTTTTTCGGAGGCTGGT |
| | | ACTTCTACATTCAGGCTTATAAAGCACTGAAGCATAAGACAGCAA |
| | | ATATGGACGTACTGATTGTGCTGGCAACCACCATTGCATTTGCCT |
| | | ACTCTTTGATTATTCTTCTAGTTGCAATGTATGAGAGAGCCAAAGT |
| | | GAACCCTATTACTTTCTTTGACACACCCCCTATGCTGTTTGTGTTT |
| | | ATTGCACTAGGCCGATGGCTGGAACATATAGCAAAGGGCAAAAC |
| | | ATCAGAGGCTCTTGCAAAGTTAATTTCACTACAAGCTACAGAAGC |
| | | AACTATTGTAACTCTTGATTCTGATAATATCCTCCTCAGTGAAGAA |
| | | CAAGTGGATGTGGAACTTGTACAACGTGGAGATATCATTAAAGTA |
| | | GTTCCAGGAGGCAAATTTCCAGTGGATGGTCGTGTTATTGAAGGA |
| | | CATTCTATGGTAGATGAGTCCCTCATCACAGGGGAGGCAATGCCT |
| | | GTGGCTAAGAAACCTGGCAGCACAGTGATTGCTGGTTCCATTAAC |
| | | CAGAACGGGTCACTGCTTATCTGCGCAACACATGTTGGAGCAGAC |
| | | ACAACCCTTTCTCAAATTGTCAAACTTGTGGAAGAGGCACAAACA |
| | | TCAAAGGCTCCTATCCAGCAGTTTGCAGACAAACTCAGTGGCTAT |
| | | TTTGTTCCTTTTATTGTTTTTGTTTCCATTGCCACCCTCTTGGTATG |
| | | GATTGTAATTGGATTTCTGAATTTTGAAATTGTGGAAACCTACTTT |
| | | CCTGGCTACAATAGAAGTATCTCCCGAACAGAAACGATAATACGA |
| | | TTTGCTTTCCAAGCCTCTATCACAGTTCTGTGTATTGCATGTCCCT |
| | | GTTCACTGGGACTGGCCACTCCAACTGCTGTGATGGTGGGTACAG |
| | | GAGTAGGTGCTCAAAATGGCATACTAATAAAAGGTGGAGAGCCA |
| | | TTGGAGATGGCTCATAAGGTAAAGGTAGTGGTATTTGATAAGACT |
| | | GGAACCATTACTCACGGAACCCCAGTGGTGAATCAAGTAAAGGTT |
| | | CTAACTGAAAGTAACAGAATATCACACCATAAAATCTTGGCCATT |
| | | GTGGGAACTGCTGAAAGTAACAGTGAACACCCTCTAGGAACAGC |
| | | CATAACCAAATATTGCAAACAGGAGCTGGACACTGAAACCTTGG |
| | | GTACCTGCATAGATTTCCAGGTTGTGCCAGGCTGTGGTATTAGCT |
| | | GTAAAGTCACCAATATTGAAGGCTTGCTACATAAGAATAACTGGA |
| | | ATATAGAGGACAATAATATTAAAAATGCATCCCTGGTTCAAATTG |
| | | ATGCCAGTAATGAACAGTCATCAACTTCGTCTTCCATGATTATTGA |
| | | TGCCCAGATCTCAAATGCTCTTAATGCTCAGCAGTATAAAGTCCT |
| | | CATTGGTAACCGGGAGTGGATGATTAGAAATGGTCTTGTCATTAA |
| | | TAACGATGTAAATGATTTCATGACTGAACATGAGAGAAAAGGTCG |
| | | GACTGCTGTATTAGTAGCAGTTGATGATGAGCTGTGTGGCTTGAT |
| | | AGCCATTGCAGACACAGTGAAGCCTGAAGCAGAACTGGCTATCC |
| | | ATATTCTGAAATCTATGGGCTTAGAAGTAGTTCTGATGACTGGAG |
| | | ACAACAGTAAAACAGCTAGATCTATTGCTTCTCAGGTTGGCATTA |
| | | CTAAGGTGTTTGCTGAAGTTCTACCTTCTCACAAGGTTGCTAAAGT |
| | | GAAGCAACTTCAAGAGGAGGGGAAACGGGTAGCAATGGTGGGAG |
| | | ATGGAATCAATGACTCCCCAGCTCTGGCAATGGCTAATGTGGGAA |
| | | TTGCTATTGGCACAGGCACAGATGTAGCCATTGAAGCAGCTGATG |
| | | TGGTTTTGATAAGGAATGATCTTCTGGATGTAGTGGCAAGTATTG |
| | | ACTTATCAAGAAAGACAGTCAAGAGGATTCGGATAAATTTTGTCT |
| | | TTGCTCTAATTTATAATCTGGTTGGAATTCCCATAGCTGCTGGAGT |
| | | TTTTATGCCCATTGGTTTGGTTTTGCAGCCCTGGATGGGATCTGCA |
| | | GCAATGGCTGCTTCATCTGTTTCTGTAGTACTTTCTTCTCTCCAGCT |
| | | CAAGTGCTATAAGAAGCCTGACCTGGAGAGGTATGAGGCACAGG |
| | | CGCATGGCCACATGAAGCCCCTGACGGCATCCCAGGTCAGTGTGC |
| | | ACATAGGCATGGATGACAGGTGGCGGGACTCCCCCAGGGCCACA |
| | | CCATGGGACCAGGTCAGCTATGTCAGCCAGGTGTCGCTGTCCTCC |
| | | CTGACGTCCGACAAGCCATCTCGGCACAGCGCTGCAGCAGACGAT |
| | | GATGGGGACAAGTGGTCTCTGCTCCTGAATGGCAGGGATGAGGA |
| | | GCAGTACATCTGA |
| 46 | TISU- | ATGGCGGCGCCTGAGCAGGAGAGACAGATCACAGCCAGAGAAGG |
| | miniATP7B- | GGCCAGTCGGAAAATCTTATCTAAGCTTTCTTTGCCTACCCGTGCC |
| | s1co | TGGGAACCAGCAATGAAGAAGAGTTTTGCTTTTGACAATGTTGGC |
| | | TATGAAGGTGGTCTGGATGGCCTGGGCCCCAGCAGCCAGGTGGCG |
| | | ACCACCTTAATCGCCATCGCAGGCATGACCTGCGCATCTTGTGTG |
| | | CATAGCATTGAAGGCATGATTAGCCAGCTGGAGGGCGTGCAGCA |
| | | GATCTCAGTCTCCCTGGCTGAGGGGACCGCCACTGTCTTGTACAA |
| | | TCCGTCTGTTATTTCCCCGGAGGAGCTGAGAGCTGCCATTGAGGA |
| | | CATGGGATTTGAAGCATCGGTGGTGTCTGAGTCATGCTCCACCAA |
| | | TCCCCTGGGAAATCACTCAGCTGGCAATTCAATGGTTCAGACCAC |
| | | CGATGGGACCCCAACCAGCGTGCAGGAAGTGGCACCCCATACCG |
| | | GCAGACTGCCTGCCAATCATGCTCCTGATATCCTCGCCAAGAGCC |
| | | CCCAGAGCACCCGCGCGGTGGCACCCCAGAAGTGTTTCCTGCAGA |
| | | TCAAGGGCATGACCTGTGCCAGCTGTGTTAGCAATATCGAAAGAA |
| | | ACCTCCAAAAAGAGGCTGGAGTTCTGAGTGTTCTAGTGGCTTTAA |
| | | TGGCCGGGAAAGCCGAGATCAAGTATGATCCCGAGGTGATTCAG |
| | | CCATTGGAGATTGCCCAGTTCATCCAGGATCTGGGCTTCGAGGCC |
| | | GCCGTGATGGAGGATTACGCCGGCTCTGATGGCAACATCGAACTG |
| | | ACCATCACCGGCATGACCTGTGCTTCCTGCGTGCATAATATCGAG |
| | | AGCAAACTGACGCGGACCAATGGAATCACCTATGCCAGCGTCGCC |
| | | CTGGCAACCTCTAAGGCTCTGGTGAAGTTTGACCCCGAAATCATT |
| | | GGACCTAGAGACATCATCAAAATTATTGAAGAGATTGGTTTCCAC |
| | | GCCAGCTTAGCTCAGCGGAATCCCAACGCACACCATCTTGACCAT |
| | | AAGATGGAAATCAAACAGTGGAAGAAGAGCTTCTTGTGTAGCCT |
| | | GGTCTTCGGGATACCCGTCATGGCACTGATGATTTACATGCTCATT |
| | | CCGTCCAACGAGCCCCACCAGTCCATGGTGCTGGACCACAACATC |
| | | ATCCCTGGGCTGTCTATCCTCAACCTGATCTTCTTTATTCTGTGCA |
| | | CATTTGTCCAGCTCTTGGGAGGCTGGTATTTCTATGTACAGGCCTA |
| | | CAAATCCCTGAGGCATCGAAGCGCCAACATGGACGTTTTAATTGT |
| | | GCTGGCTACATCCATTGCCTACGTGTACTCCCTGGTGATCTTAGTG |
| | | GTTGCTGTGGCTGAAAAGGCTGAGAGAAGCCCTGTCACCTTCTTC |
| | | GACACCCCCCCCATGCTCTTCGTGTTTATTGCACTGGGTCGCTGGC |
| | | TGGAGCATTTAGCCAAGTCCAAGACATCCGAGGCTCTGGCCAAGC |
| | | TGATGTCTCTACAGGCCACGGAGGCCACCGTGGTGACTCTGGGTG |
| | | AAGACAATTTGATAATACGCGAGGAACAGGTGCCTATGGAGCTTG |
| | | TGCAGCGCGGAGATATCGTCAAGGTGGTGCCCGGCGGCAAGTTCC |
| | | CAGTGGATGGAAAAGTGTTAGAGGGCAATACCATGGCCGATGAA |
| | | TCCCTCATCACTGGCGAGGCAATGCCGGTGACCAAGAAACCTGGA |
| | | AGTACTGTCATTGCAGGCAGCATCAATGCCCATGGGAGCGTCCTG |
| | | ATCAAGGCCACCCACGTGGGCAATGATACAACTCTCGCCCAGATC |
| | | GTCAAGCTAGTGGAAGAGGCTCAGATGTCTAAAGCTCCAATCCAG |
| | | CAACTGGCTGACCGCTTCAGCGGCTACTTTGTCCCCTTTATCATCA |
| | | TCATGAGCACCCTCACCCTGGTGGTGTGGATAGTCATAGGCTTCA |
| | | TTGACTTTGGCGTGGTGCAGAGATACTTCCCCAACCCTAACAAAC |
| | | ATATCTCCCAAACCGAGGTCATTATCAGATTCGCCTTTCAGACATC |
| | | TATCACTGTGCTGTGCATAGCCTGCCCTTGCTCTCTGGGCCTGGCC |
| | | ACCCCCACCGCCGTGATGGTTGGAACCGGGGTCGCTGCACAAAAT |
| | | GGGATCCTGATAAAAGGGGGCAAACCCCTGGAAATGGCACATAA |
| | | GATTAAAACAGTTATGTTTGATAAGACAGGCACCATTACTCACGG |
| | | TGTCCCTCGAGTCATGAGAGTGCTGCTGCTGGGGGACGTGGCTAC |
| | | ACTGCCACTGCGCAAGGTGCTGGCTGTGGTGGGCACAGCGGAGG |
| | | CCTCCTCAGAACACCCATTAGGCGTAGCTGTCACAAAATACTGTA |
| | | AGGAGGAGCTGGGGACAGAGACCCTCGGCTATTGTACAGACTTCC |
| | | AGGCCGTGCCTGGCTGCGGCATCGGCTGTAAGGTCAGCAATGTGG |
| | | AGGGCATCCTGGCACACAGCGAGAGACCCCTCTCTGCCCCAGCCT |
| | | CCCACCTGAATGAAGCTGGGTCTCTGCCTGCTGAGAAGGACGCCG |
| | | TCCCTCAGACCTTCTCCGTGTTGATTGGGAACAGAGAGTGGCTTA |
| | | GAAGGAATGGCCTGACCATCAGCTCTGACGTCTCGGATGCCATGA |
| | | CAGATCACGAGATGAAAGGCCAGACAGCCATACTGGTTGCCATC |
| | | GACGGCGTGCTCTGTGGCATGATTGCCATCGCTGATGCTGTTAAA |
| | | CAGGAAGCTGCGCTGGCAGTCCACACCCTCCAGTCCATGGGAGTG |
| | | GACGTGGTCCTGATTACTGGAGACAACAGGAAAACCGCTCGGGC |
| | | CATCGCTACCCAAGTGGGGATAAATAAGGTTTTTGCAGAAGTCTT |
| | | ACCCTCCCACAAAGTAGCCAAAGTGCAGGAGCTCCAGAACAAGG |
| | | GTAAAAAGGTGGCAATGGTGGGAGATGGTGTGAATGACAGCCCA |
| | | GCTCTCGCGCAGGCTGACATGGGCGTAGCCATAGGTACTGGAACA |
| | | GATGTCGCGATTGAAGCCGCTGACGTAGTCCTCATCCGAAACGAT |
| | | TTGCTGGATGTGGTGGCCAGCATCCATCTGTCCAAGCGTACCGTG |
| | | CGCCGGATCCGCATCAACCTGGTCTTGGCCCTCATCTATAATCTCG |
| | | TGGGCATCCCCATCGCTGCTGGGGTCTTCATGCCCATCGGCATTGT |
| | | GCTGCAGCCCTGGATGGGAAGCGCTGCCATGGCTGCTTCTTCTGT |
| | | CTCCGTTGTGCTGTCCAGCCTACAGCTGAAGTGCTATAAAAAGCC |
| | | AGACCTCGAGCGCTACGAAGCCCAGGCGCATGGCCATATGAAGC |
| | | CCCTCACGGCTTCCCAGGTCTCGGTTCACATTGGGATGGATGACA |
| | | GATGGCGGGACTCCCCACGCGCCACACCCTGGGACCAGGTATCCT |
| | | ATGTGAGCCAGGTGAGCCTCAGCTCCCTGACATCAGATAAACCGT |
| | | CTAGGCACTCAGCTGCCGCAGATGATGACGGTGACAAATGGAGC |
| | | CTCCTCCTGAACGGCCGCGACGAGGAGCAGTACATCTAA |
| 47 | TISU- | ATGGCGGCGCCTGAGCAGGAGAGACAGATCACAGCCAGAGAAGG |
| | miniATP7B- | GGCCAGTCGGAAAATCTTATCTAAGCTTTCTTTGCCTACCCGTGCC |
| | s1co3 | TGGGAACCAGCAATGAAGAAGAGTTTTGCTTTTGACAATGTTGGC |
| | | TATGAAGGTGGTCTGGATGGCCTGGGCCCTAGCAGCCAGGTGGCT |
| | | ACCACCCTGATTGCCATTGCCGGCATGACCTGCGCCAGCTGCGTG |
| | | CATAGCATTGAGGGCATGATTAGCCAGCTGGAGGGCGTGCAGCA |
| | | GATTTCCGTGAGCCTGGCTGAGGGCACCGCTACCGTGCTGTATAA |
| | | TCCCTCCGTGATTAGCCCCGAAGAACTGCGCGCTGCCATTGAAGA |
| | | TATGGGCTTTGAAGCCTCCGTGGTGTCCGAAAGCTGCAGCACCAA |
| | | TCCCCTGGGCAATCATTCCGCCGGCAATAGCATGGTGCAGACCAC |
| | | CGACGGCACCCCCACCTCCGTGCAGGAAGTGGCTCCCCATACCGG |
| | | CCGCCTGCCCGCCAATCATGCCCCTGATATTCTGGCTAAAAGCCC |
| | | CCAGAGCACCCGCGCCGTGGCTCCTCAGAAATGCTTTCTGCAGAT |
| | | TAAGGGCATGACCTGCGCTAGCTGCGTGAGCAATATTGAACGCAA |
| | | TCTGCAGAAAGAAGCCGGCGTGCTGTCCGTGCTGGTGGCCCTGAT |
| | | GGCCGGCAAAGCTGAAATCAAATATGATCCCGAAGTGATTCAGCC |
| | | CCTGGAAATTGCCCAGTTCATTCAGGATCTGGGCTTTGAGGCTGC |
| | | CGTGATGGAAGATTATGCCGGCTCCGACGGCAATATTGAACTGAC |
| | | CATTACCGGCATGACCTGCGCCAGCTGCGTGCATAATATTGAAAG |
| | | CAAACTGACCCGCACCAACGGCATTACCTATGCTTCCGTGGCCCT |
| | | GGCTACCAGCAAAGCCCTGGTGAAATTTGATCCTGAAATTATCGG |
| | | CCCCCGCGATATTATCAAAATTATTGAAGAAATCGGCTTTCACGC |
| | | CAGCCTGGCTCAGCGCAATCCCAATGCCCATCATCTGGATCATAA |
| | | GATGGAAATCAAACAGTGGAAGAAAAGCTTTCTGTGCAGCCTGGT |
| | | GTTCGGCATTCCTGTGATGGCCCTGATGATCTATATGCTGATTCCC |
| | | AGCAATGAACCCCATCAGAGCATGGTGCTGGATCATAATATTATT |
| | | CCCGGCCTGAGCATTCTGAATCTGATTTTCTTTATCCTGTGCACCT |
| | | TTGTGCAGCTGCTGGGCGGCTGGTATTTCTATGTGCAGGCTTATAA |
| | | AAGCCTGCGCCACCGCTCCGCCAATATGGATGTGCTGATTGTGCT |
| | | GGCCACCAGCATTGCTTATGTCTATAGCCTGGTCATTCTGGTGGTG |
| | | GCCGTGGCCGAGAAAGCTGAACGCAGCCCCGTGACCTTCTTTGAT |
| | | ACCCCTCCCATGCTGTTCGTGTTTATCGCTCTGGGCCGCTGGCTGG |
| | | AACATCTGGCCAAAAGCAAAACCTCCGAAGCCCTGGCTAAACTG |
| | | ATGAGCCTGCAGGCCACCGAAGCCACCGTGGTGACCCTGGGCGA |
| | | AGATAATCTGATTATCCGCGAAGAACAGGTGCCTATGGAACTGGT |
| | | GCAGCGCGGCGATATTGTGAAAGTGGTGCCCGGCGGCAAATTTCC |
| | | CGTGGACGGCAAAGTGCTGGAGGGCAATACCATGGCTGATGAAT |
| | | CCCTGATTACCGGCGAAGCTATGCCTGTGACCAAGAAACCCGGCA |
| | | GCACCGTGATTGCCGGCAGCATTAATGCTCACGGCTCCGTGCTGA |
| | | TCAAGGCTACCCATGTCGGCAATGATACCACCCTGGCCCAGATTG |
| | | TGAAACTGGTGGAAGAAGCCCAGATGAGCAAAGCCCCTATTCAG |
| | | CAGCTGGCTGACCGCTTTTCCGGCTATTTTGTGCCCTTTATCATTA |
| | | TTATGAGCACCCTGACCCTGGTGGTGTGGATTGTGATCGGCTTCAT |
| | | TGATTTCGGCGTGGTGCAGAGATATTTTCCTAATCCCAACAAACA |
| | | TATTAGCCAGACCGAAGTGATTATCCGCTTTGCTTTTCAGACCAGC |
| | | ATTACCGTGCTGTGCATTGCTTGCCCCTGCAGCCTGGGCCTGGCTA |
| | | CCCCCACCGCCGTCATGGTCGGCACCGGCGTGGCTGCCCAGAACG |
| | | GCATTCTGATTAAGGGCGGCAAACCTCTGGAAATGGCCCACAAAA |
| | | TCAAAACCGTGATGTTTGATAAAACCGGCACCATTACCCACGGCG |
| | | TGCCCCGCGTGATGCGCGTGCTGCTGCTGGGCGATGTGGCTACCC |
| | | TGCCCCTGCGCAAAGTGCTGGCCGTGGTCGGCACCGCCGAAGCTA |
| | | GCTCCGAACATCCCCTGGGCGTGGCCGTGACCAAATATTGCAAAG |
| | | AAGAACTGGGCACCGAAACCCTGGGCTATTGCACCGATTTTCAGG |
| | | CCGTCCCTGGCTGCGGCATCGGCTGCAAAGTGAGCAATGTGGAGG |
| | | GCATTCTGGCCCATTCCGAAAGACCCCTGTCCGCCCCTGCTAGCC |
| | | ATCTGAATGAAGCCGGCAGCCTGCCTGCTGAGAAAGATGCCGTGC |
| | | CCCAGACCTTTTCCGTGCTGATCGGCAACCGCGAATGGCTGCGCC |
| | | GCAACGGCCTGACCATTAGCTCCGATGTCTCCGATGCCATGACCG |
| | | ATCATGAAATGAAAGGCCAGACCGCCATTCTGGTGGCCATTGACG |
| | | GCGTGCTGTGCGGCATGATTGCCATTGCCGATGCCGTGAAACAGG |
| | | AGGCTGCCCTGGCTGTGCATACCCTGCAGAGCATGGGCGTGGATG |
| | | TGGTGCTGATTACCGGCGATAACCGCAAAACCGCTCGCGCCATTG |
| | | CCACCCAGGTCGGCATTAACAAAGTGTTTGCTGAAGTGCTGCCCA |
| | | GCCATAAAGTGGCTAAAGTGCAGGAACTGCAGAACAAAGGCAAG |
| | | AAAGTGGCTATGGTCGGCGACGGCGTGAATGATAGCCCTGCCCTG |
| | | GCCCAGGCCGATATGGGCGTGGCTATCGGCACCGGCACCGATGTG |
| | | GCTATTGAGGCTGCCGATGTGGTGCTGATCCGCAATGATCTGCTG |
| | | GATGTGGTGGCTAGCATTCATCTGAGCAAACGCACCGTCCGCCGC |
| | | ATCCGCATTAATCTGGTGCTGGCCCTGATCTATAATCTGGTCGGCA |
| | | TTCCCATCGCCGCCGGCGTGTTTATGCCCATCGGCATTGTGCTGCA |
| | | GCCCTGGATGGGCAGCGCCGCTATGGCCGCTAGCTCCGTGTCCGT |
| | | GGTGCTGAGCAGCCTGCAGCTGAAATGCTATAAGAAACCCGATCT |
| | | GGAAAGATATGAAGCTCAGGCTCACGGCCATATGAAACCCCTGA |
| | | CCGCCAGCCAGGTGTCCGTGCATATCGGCATGGATGACCGCTGGC |
| | | GCGATAGCCCCCGCGCCACCCCTTGGGATCAGGTGAGCTATGTGA |
| | | GCCAGGTGTCCCTGAGCTCCCTGACCTCCGATAAACCCTCCCGCC |
| | | ATTCCGCCGCTGCCGATGATGACGGCGATAAGTGGAGCCTGCTGC |
| | | TGAACGGCCGCGATGAAGAACAGTATATCTAA |
| 48 | TISU- | ATGGCGGCGCCTGAGCAGGAGAGACAGATCACAGCCAGAGAAGG |
| | miniATP7Bv. | GGCCAGTCGGAAAATCTTATCTAAGCTTTCTTTGCCTACCCGTGCC |
| | co | TGGGAACCAGCAATGAAGAAGAGTTTTGCTTTTGACAATGTTGGC |
| | | TATGAAGGTGGTCTGGATGGCCTGGGCCCCAGCAGCCAGCCCCAG |
| | | AAGTGTTTCCTGCAGATCAAGGGCATGACCTGTGCCAGCTGTGTT |
| | | AGCAATATCGAAAGAAACCTCCAAAAAGAGGCTGGAGTTCTGAG |
| | | TGTTCTAGTGGCTTTAATGGCCGGGAAAGCCGAGATCAAGTATGA |
| | | TCCCGAGGTGATTCAGCCATTGGAGATTGCCCAGTTCATCCAGGA |
| | | TCTGGGCTTCGAGGCCGCCGTGATGGAGGATTACGCCGGCTCTGA |
| | | TGGCAACATCGAACTGACCATCACCGGCATGACCTGTGCTTCCTG |
| | | CGTGCATAATATCGAGAGCAAACTGACGCGGACCAATGGAATCA |
| | | CCTATGCCAGCGTCGCCCTGGCAACCTCTAAGGCTCTGGTGAAGT |
| | | TTGACCCCGAAATCATTGGACCTAGAGACATCATCAAAATTATTG |
| | | AAGAGATTGGTTTCCACGCCAGCTTAGCTCAGCGGAATCCCAACG |
| | | CACACCATCTTGACCATAAGATGGAAATCAAACAGTGGAAGAAG |
| | | AGCTTCTTGTGTAGCCTGGTCTTCGGGATACCCGTCATGGCACTGA |
| | | TGATTTACATGCTCATTCCGTCCAACGAGCCCCACCAGTCCATGGT |
| | | GCTGGACCACAACATCATCCCTGGGCTGTCTATCCTCAACCTGAT |
| | | CTTCTTTATTCTGTGCACATTTGTCCAGCTCTTGGGAGGCTGGTAT |
| | | TTCTATGTACAGGCCTACAAATCCCTGAGGCATCGAAGCGCCAAC |
| | | ATGGACGTTTTAATTGTGCTGGCTACATCCATTGCCTACGTGTACT |
| | | CCCTGGTGATCTTAGTGGTTGCTGTGGCTGAAAAGGCTGAGAGAA |
| | | GCCCTGTCACCTTCTTCGACACCCCCCCCATGCTCTTCGTGTTTAT |
| | | TGCACTGGGTCGCTGGCTGGAGCATTTAGCCAAGTCCAAGACATC |
| | | CGAGGCTCTGGCCAAGCTGATGTCTCTACAGGCCACGGAGGCCAC |
| | | CGTGGTGACTCTGGGTGAAGACAATTTGATAATACGCGAGGAACA |
| | | GGTGCCTATGGAGCTTGTGCAGCGCGGAGATATCGTCAAGGTGGT |
| | | GCCCGGCGGCAAGTTCCCAGTGGATGGAAAAGTGTTAGAGGGCA |
| | | ATACCATGGCCGATGAATCCCTCATCACTGGCGAGGCAATGCCGG |
| | | TGACCAAGAAACCTGGAAGTACTGTCATTGCAGGCAGCATCAATG |
| | | CCCATGGGAGCGTCCTGATCAAGGCCACCCACGTGGGCAATGATA |
| | | CAACTCTCGCCCAGATCGTCAAGCTAGTGGAAGAGGCTCAGATGT |
| | | CTAAAGCTCCAATCCAGCAACTGGCTGACCGCTTCAGCGGCTACT |
| | | TTGTCCCCTTTATCATCATCATGAGCACCCTCACCCTGGTGGTGTG |
| | | GATAGTCATAGGCTTCATTGACTTTGGCGTGGTGCAGAGATACTT |
| | | CCCCAACCCTAACAAACATATCTCCCAAACCGAGGTCATTATCAG |
| | | ATTCGCCTTTCAGACATCTATCACTGTGCTGTGCATAGCCTGCCCT |
| | | TGCTCTCTGGGCCTGGCCACCCCCACCGCCGTGATGGTTGGAACC |
| | | GGGGTCGCTGCACAAAATGGGATCCTGATAAAAGGGGGCAAACC |
| | | CCTGGAAATGGCACATAAGATTAAAACAGTTATGTTTGATAAGAC |
| | | AGGCACCATTACTCACGGTGTCCCTCGAGTCATGAGAGTGCTGCT |
| | | GCTGGGGGACGTGGCTACACTGCCACTGCGCAAGGTGCTGGCTGT |
| | | GGTGGGCACAGCGGAGGCCTCCTCAGAACACCCATTAGGCGTAG |
| | | CTGTCACAAAATACTGTAAGGAGGAGCTGGGGACAGAGACCCTC |
| | | GGCTATTGTACAGACTTCCAGGCCGTGCCTGGCTGCGGCATCGGC |
| | | TGTAAGGTCAGCAATGTGGAGGGCATCCTGGCACACAGCGAGAG |
| | | ACCCCTCTCTGCCCCAGCCTCCCACCTGAATGAAGCTGGGTCTCTG |
| | | CCTGCTGAGAAGGACGCCGTCCCTCAGACCTTCTCCGTGTTGATT |
| | | GGGAACAGAGAGTGGCTTAGAAGGAATGGCCTGACCATCAGCTC |
| | | TGACGTCTCGGATGCCATGACAGATCACGAGATGAAAGGCCAGA |
| | | CAGCCATACTGGTTGCCATCGACGGCGTGCTCTGTGGCATGATTG |
| | | CCATCGCTGATGCTGTTAAACAGGAAGCTGCGCTGGCAGTCCACA |
| | | CCCTCCAGTCCATGGGAGTGGACGTGGTCCTGATTACTGGAGACA |
| | | ACAGGAAAACCGCTCGGGCCATCGCTACCCAAGTGGGGATAAAT |
| | | AAGGTTTTTGCAGAAGTCTTACCCTCCCACAAAGTAGCCAAAGTG |
| | | CAGGAGCTCCAGAACAAGGGTAAAAAGGTGGCAATGGTGGGAGA |
| | | TGGTGTGAATGACAGCCCAGCTCTCGCGCAGGCTGACATGGGCGT |
| | | AGCCATAGGTACTGGAACAGATGTCGCGATTGAAGCCGCTGACGT |
| | | AGTCCTCATCCGAAACGATTTGCTGGATGTGGTGGCCAGCATCCA |
| | | TCTGTCCAAGCGTACCGTGCGCCGGATCCGCATCAACCTGGTCTT |
| | | GGCCCTCATCTATAATCTCGTGGGCATCCCCATCGCTGCTGGGGTC |
| | | TTCATGCCCATCGGCATTGTGCTGCAGCCCTGGATGGGAAGCGCT |
| | | GCCATGGCTGCTTCTTCTGTCTCCGTTGTGCTGTCCAGCCTACAGC |
| | | TGAAGTGCTATAAAAAGCCAGACCTCGAGCGCTACGAAGCCCAG |
| | | GCGCATGGCCATATGAAGCCCCTCACGGCTTCCCAGGTCTCGGTT |
| | | CACATTGGGATGGATGACAGATGGCGGGACTCCCCACGCGCCAC |
| | | ACCCTGGGACCAGGTATCCTATGTGAGCCAGGTGAGCCTCAGCTC |
| | | CCTGACATCAGATAAACCGTCTAGGCACTCAGCTGCCGCAGATGA |
| | | TGACGGTGACAAATGGAGCCTCCTCCTGAACGGCCGCGACGAGG |
| | | AGCAGTACATCTAA |

TABLE 10
Sequences for additional genetic elements.

| SEQ | | |
|---|---|---|
| ID | | |
| NO | Element | Sequence |
| 49 | WPRE250 | AATCAACCTCTGGATTACAAAATTTGTGAAAGATTGACTGGTATTCT |
| | | TAACTATGTTGCTCCTTTTACGCTATGTGGATACGCTGCTTTAATGC |
| | | CTTTGTATCATGCTATTGCTTCCCGTATGGCTTTCATTTTCTCCTCCT |
| | | TGTATAAATCCTGGTTGCTGTCTCTTTATGAGGAGTTGTGGCCCGTT |
| | | GTCAGGCAACGTGGCGTGGTGTGCACTGTGTTTGCTGACGCAACCC |
| | | CCACTGGTTGGGGCA |
| 50 | WPRE3 | AATCAACCTCTGGATTACAAAATTTGTGAAAGATTGACTGGTATTCT |
| | | TAACTATGTTGCTCCTTTTACGCTATGTGGATACGCTGCTTTAATGC |
| | | CTTTGTATCATGCTATTGCTTCCCGTATGGCTTTCATTTTCTCCTCCT |
| | | TGTATAAATCCTGGTTAGTTCTTGCCACGGCGGAACTCATCGCCGCC |
| | | TGCCTTGCCCGCTGCTGGACAGGGGCTCGGCTGTTGGGCACTGACA |
| | | ATTCCGTGGTGTTCA |
| 51 | sPolyA | AATAAAAGATCTTTATTTTCATTAGATCTGTGTGTTGGTTTTTTGTGT |
| | | G |
| 52 | SV40 | CAGACATGATAAGATACATTGATGAGTTTGGACAAACCACAACTAG |
| | late pA | AATGCAGTGAAAAAAATGCTTTATTTGTGAAATTTGTGATGCTATTG |
| | | CTTTATTTGTAACCATTATAAGCTGCAATAAACAAGTTAACAACAA |
| | | CAATTGCATTCATTTTATGTTTCAGGTTCAGGGGGAGGTGTGGGAG |
| | | GTTTTTTAAAGCAAGTAAAACCTCTACAAATGTGGTA |
| 53 | 4xmiR142 | TCCATAAAGTAGGAAACACTACATCATCCATAAAGTAGGAAACACT |
| | | ACATCATCCATAAAGTAGGAAACACTACATCATCCATAAAGTAGGA |
| | | AACACTACA |
| 116 | 5′ ITR | TTGGCCACTCCCTCTCTGCGCGCTCGCTCGCTCACTGAGGCCGGGCG |
| | (used in | ACCAAAGGTCGCCCGACGCCCGGGCTTTGCCCGGGCGGCCTCAGTG |
| | A1-A62) | AGCGAGCGAGCGCGCAGAGAGGGAGTGGCCAACTCCATCACTAGG |
| | | GGTTCCT |
| 117 | 3′ ITR | AGGAACCCCTAGTGATGGAGTTGGCCACTCCCTCTCTGCGCGCTCG |
| | (used in | CTCGCTCACTGAGGCCGGGCGACCAAAGGTCGCCCGACGCCCGGGC |
| | A1-A62) | TTTGCCCGGGCGGCCTCAGTGAGCGAGCGAGCGCGCAGAGAGGGA |
| | | GTGGCCAA |

TABLE 11
Construct sequences (5′ and 3′ ITR inclusive). SN: = SEQ ID NO.

| SN: | Construct | Sequence |
|---|---|---|
| 54 | A1 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGCGGCCGCACGCGTCGCCACCCCCTCCACCTTGGACA |
| | | CAGGACGCTGTGGTTTCTGAGCCAGGTACAATGACTCCTTTCGGTA |
| | | AGTGCAGTGGAAGCTGTACACTGCCCAGGCAAAGCGTCCGGGCAG |
| | | CGTAGGCGGGCGACTCAGATCCCAGCCAGTGGACTTAGCCCCTGTT |
| | | TGCTCCTCCGATAACTGGGGTGACCTTGGTTAATATTCACCAGCAG |
| | | CCTCCCCCGTTGCCCCTCTGGATCCACTGCTTAAATACGGACGAGG |
| | | ACAGGGCCCTGTCTCCTCAGCTTCAGGCACCACCACTGACCTGGGA |
| | | CAGTGAAGCGGCCGCCACCATGCCTGAACAGGAGAGACAGATCAC |
| | | AGCCAGAGAAGGGGCCAGTCGGAAAATCTTATCTAAGCTTTCTTTG |
| | | CCTACCCGTGCCTGGGAACCAGCAATGAAGAAGAGTTTTGCTTTTG |
| | | ACAATGTTGGCTATGAAGGTGGTCTGGATGGCCTGGGCCCTTCTTC |
| | | TCAGGTGGCCACCAGCACAGTCAGGATCTTGGGCATGACTTGCCA |
| | | GTCATGTGTGAAGTCCATTGAGGACAGGATTTCCAATTTGAAAGGC |
| | | ATCATCAGCATGAAGGTTTCCCTGGAACAAGGCAGTGCCACTGTG |
| | | AAATATGTGCCATCGGTTGTGTGCCTGCAACAGGTTTGCCATCAAA |
| | | TTGGGGACATGGGCTTCGAGGCCAGCATTGCAGAAGGAAAGGCAG |
| | | CCTCCTGGCCCTCAAGGTCCTTGCCTGCCCAGGAGGCTGTGGTCAA |
| | | GCTCCGGGTGGAGGGCATGACCTGCCAGTCCTGTGTCAGCTCCATT |
| | | GAAGGCAAGGTCCGGAAACTGCAAGGAGTAGTGAGAGTCAAAGT |
| | | CTCACTCAGCAACCAAGAGGCCGTCATCACTTATCAGCCTTATCTC |
| | | ATTCAGCCCGAAGACCTCAGGGACCATGTAAATGACATGGGATTT |
| | | GAAGCTGCCATCAAGAGCAAAGTGGCTCCCTTAAGCCTGGGACCA |
| | | ATTGATATTGAGCGGTTACAAAGCACTAACCCAAAGAGACCTTTAT |
| | | CTTCTGCTAACCAGAATTTTAATAATTCTGAGACCTTGGGGCACCA |
| | | AGGAAGCCATGTGGTCACCCTCCAACTGAGAATAGATGGAATGCA |
| | | TTGTAAGTCTTGCGTCTTGAATATTGAAGAAAATATTGGCCAGCTC |
| | | CTAGGGGTTCAAAGTATTCAAGTCTCCTTGGAGAACAAAACTGCCC |
| | | AAGTAAAGTATGACCCTTCTTGTACCAGCCCAGTGGCTCTGCAGAG |
| | | GGCTATCGAGGCACTTCCACCTGGGAATTTTAAAGTTTCTCTTCCT |
| | | GATGGAGCCGAAGGGAGTGGGACAGATCACAGGTCTTCCAGTTCT |
| | | CATTCCCCTGGCTCCCCACCGAGAAACCAGGTCCAGGGCACATGC |
| | | AGTACCACTCTGATTGCCATTGCCGGCATGACCTGTGCATCCTGTG |
| | | TCCATTCCATTGAAGGCATGATCTCCCAACTGGAAGGGGTGCAGC |
| | | AAATATCGGTGTCTTTGGCCGAAGGGACTGCAACAGTTCTTTATAA |
| | | TCCCGCTGTAATTAGCCCAGAAGAACTCAGAGCTGCTATAGAAGA |
| | | CATGGGATTTGAGGCTTCAGTCGTTTCTGAAAGCTGTTCTACTAAC |
| | | CCTCTTGGAAACCACAGTGCTGGGAATTCCATGGTGCAAACTACA |
| | | GATGGTACACCTACATCTCTGCAGGAAGTGGCTCCCCACACTGGG |
| | | AGGCTCCCTGCAAACCATGCCCCGGACATCTTGGCAAAGTCCCCAC |
| | | AATCAACCAGAGCAGTGGCACCGCAGAAGTGCTTCTTACAGATCA |
| | | AAGGCATGACCTGTGCATCCTGTGTGTCTAACATAGAAAGGAATCT |
| | | GCAGAAAGAAGCTGGTGTTCTCTCCGTGTTGGTTGCCTTGATGGCA |
| | | GGAAAGGCAGAGATCAAGTATGACCCAGAGGTCATCCAGCCCCTC |
| | | GAGATAGCTCAGTTCATCCAGGACCTGGGTTTTGAGGCAGCAGTC |
| | | ATGGAGGACTACGCAGGCTCCGATGGCAACATTGAGCTGACAATC |
| | | ACAGGGATGACCTGCGCGTCCTGTGTCCACAACATAGAGTCCAAA |
| | | CTCACGAGGACAAATGGCATCACTTATGCCTCCGTTGCCCTTGCCA |
| | | CCAGCAAAGCCCTTGTTAAGTTTGACCCGGAAATTATCGGTCCACG |
| | | GGATATTATCAAAATTATTGAGGAAATTGGCTTTCATGCTTCCCTG |
| | | GCCCAGAGAAACCCCAACGCTCATCACTTGGACCACAAGATGGAA |
| | | ATAAAGCAGTGGAAGAAGTCTTTCCTGTGCAGCCTGGTGTTTGGCA |
| | | TCCCTGTCATGGCCTTAATGATCTATATGCTGATACCCAGCAACGA |
| | | GCCCCACCAGTCCATGGTCCTGGACCACAACATCATTCCAGGACTG |
| | | TCCATTCTAAATCTCATCTTCTTTATCTTGTGTACCTTTGTCCAGCT |
| | | CCTCGGTGGGTGGTACTTCTACGTTCAGGCCTACAAATCTCTGAGA |
| | | CACAGGTCAGCCAACATGGACGTGCTCATCGTCCTGGCCACAAGC |
| | | ATTGCTTATGTTTATTCTCTGGTCATCCTGGTGGTTGCTGTGGCTGA |
| | | GAAGGCGGAGAGGAGCCCTGTGACATTCTTCGACACGCCCCCCAT |
| | | GCTCTTTGTGTTCATTGCCCTGGGCCGGTGGCTGGAACACTTGGCA |
| | | AAGAGCAAAACCTCAGAAGCCCTGGCTAAACTCATGTCTCTCCAA |
| | | GCCACAGAAGCCACCGTTGTGACCCTTGGTGAGGACAATTTAATC |
| | | ATCAGGGAGGAGCAAGTCCCCATGGAGCTGGTGCAGCGGGGCGAT |
| | | ATCGTCAGGGTGGTCCCTGGGGGAAAGTTTCCAGTGGATGGGAAA |
| | | GTCCTGGAAGGCAATACCATGGCTGATGAGTCCCTCATCACAGGA |
| | | GAAGCCATGCCAGTCACTAAGAAACCCGGAAGCACTGTAATTGCG |
| | | GGGTCTATAAATGCACATGGCTCTGTGCTCATTAAAGCTACCCACG |
| | | TGGGCAATGACACCACTTTGGCTCAGATTGTGAAACTGGTGGAAG |
| | | AGGCTCAGATGTCAAAGGCACCCATTCAGCAGCTGGCTGACCGGT |
| | | TTAGTGGATATTTTGTCCCATTTATCATCATCATGTCAACTTTGACG |
| | | TTGGTGGTATGGATTGTAATCGGTTTTATCGATTTTGGTGTTGTTCA |
| | | GAAATACTTTCCTAACCCCAACAAGCACATCTCCCAGACAGAGGT |
| | | GATCATCCGGTTTGCTTTCCAGACGTCCATCACGGTGCTGTGCATT |
| | | GCCTGCCCCTGCTCCCTGGGGCTGGCCACGCCCACGGCTGTCATGG |
| | | TGGGCACCGGGGTGGCCGCGCAGAACGGCATCCTCATCAAGGGAG |
| | | GCAAGCCCCTGGAGATGGCGCACAAGATAAAGACTGTGATGTTTG |
| | | ACAAGACTGGCACCATTACCCATGGCGTCCCCAGGGTCATGCGGG |
| | | TGCTCCTGCTGGGGGATGTGGCCACACTGCCCCTCAGGAAGGTTCT |
| | | GGCTGTGGTGGGGACTGCGGAGGCCAGCAGTGAACACCCCTTGGG |
| | | CGTGGCAGTCACCAAATACTGTAAAGAGGAACTTGGAACAGAGAC |
| | | CTTGGGATACTGCACGGACTTCCAGGCAGTGCCAGGCTGTGGAATT |
| | | GGGTGCAAAGTCAGCAACGTGGAAGGCATCCTGGCCCACAGTGAG |
| | | CGCCCTTTGAGTGCACCGGCCAGTCACCTGAATGAGGCTGGCAGC |
| | | CTTCCCGCAGAAAAAGATGCAGCCCCCCAGACCTTCTCTGTGCTGA |
| | | TTGGAAACCGTGAGTGGCTGAGGCGCAACGGTTTAACCATTTCTAG |
| | | CGATGTCAGTGACGCTATGACAGACCACGAGATGAAAGGACAGAC |
| | | AGCCATCCTGGTGGCTATTGACGGTGTGCTCTGTGGGATGATCGCA |
| | | ATCGCAGACGCTGTCAAGCAGGAGGCTGCCCTGGCTGTGCACACG |
| | | CTGCAGAGCATGGGTGTGGACGTGGTTCTGATCACGGGGGACAAC |
| | | CGGAAGACAGCCAGAGCTATTGCCACCCAGGTTGGCATCAACAAA |
| | | GTCTTTGCAGAGGTGCTGCCTTCGCACAAGGTGGCCAAGGTCCAG |
| | | GAGCTCCAGAATAAAGGGAAGAAAGTCGCCATGGTGGGGGATGG |
| | | GGTCAATGACTCCCCGGCCTTGGCCCAGGCAGACATGGGTGTGGC |
| | | CATTGGCACCGGCACGGATGTGGCCATCGAGGCAGCCGACGTCGT |
| | | CCTTATCAGAAATGATTTGCTGGATGTGGTGGCTAGCATTCACCTT |
| | | TCCAAGAGGACTGTCCGAAGGATACGCATCAACCTGGTCCTGGCA |
| | | CTGATTTATAACCTGGTTGGGATACCCATTGCAGCAGGTGTCTTCA |
| | | TGCCCATCGGCATTGTGCTGCAGCCCTGGATGGGCTCAGCGGCCAT |
| | | GGCAGCCTCCTCTGTGTCTGTGGTGCTCTCATCCCTGCAGCTCAAG |
| | | TGCTATAAGAAGCCTGACCTGGAGAGGTATGAGGCACAGGCGCAT |
| | | GGCCACATGAAGCCCCTGACGGCATCCCAGGTCAGTGTGCACATA |
| | | GGCATGGATGACAGGTGGCGGGACTCCCCCAGGGCCACACCATGG |
| | | GACCAGGTCAGCTATGTCAGCCAGGTGTCGCTGTCCTCCCTGACGT |
| | | CCGACAAGCCATCTCGGCACAGCGCTGCAGCAGACGATGATGGGG |
| | | ACAAGTGGTCTCTGCTCCTGAATGGCAGGGATGAGGAGCAGTACA |
| | | TCTGAAATAAAAGATCTTTATTTTCATTAGATCTGTGTGTTGGTTTT |
| | | TTGTGTGGATATCGGATCCCGTGCGGACCGAGCGGCCGCAGGAAC |
| | | CCCTAGTGATGGAGTTGGCCACTCCCTCTCTGCGCGCTCGCTCGCT |
| | | CACTGAGGCCGGGCGACCAAAGGTCGCCCGACGCCCGGGCTTTGC |
| | | CCGGGCGGCCTCAGTGAGCGAGCGAGCGCGCAGAGAGGGAGTGG |
| | | CCAA |
| 55 | A2 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGCGGCCGCACGCGTCGCCACCCCCTCCACCTTGGACA |
| | | CAGGACGCTGTGGTTTCTGAGCCAGGTACAATGACTCCTTTCGGTA |
| | | AGTGCAGTGGAAGCTGTACACTGCCCAGGCAAAGCGTCCGGGCAG |
| | | CGTAGGCGGGCGACTCAGATCCCAGCCAGTGGACTTAGCCCCTGTT |
| | | TGCTCCTCCGATAACTGGGGTGACCTTGGTTAATATTCACCAGCAG |
| | | CCTCCCCCGTTGCCCCTCTGGATCCACTGCTTAAATACGGACGAGG |
| | | ACAGGGCCCTGTCTCCTCAGCTTCAGGCACCACCACTGACCTGGGA |
| | | CAGTGAAGCGGCCGCCACCATGCCTGAACAGGAGAGACAGATCAC |
| | | AGCCAGAGAAGGGGCCAGTCGGAAAATCTTATCTAAGCTTTCTTTG |
| | | CCTACCCGTGCCTGGGAACCAGCAATGAAGAAGAGTTTTGCTTTTG |
| | | ACAATGTTGGCTATGAAGGTGGTCTGGATGGCCTGGGCCCTTCTTC |
| | | TCAGCCGCAGAAGTGCTTCTTACAGATCAAAGGCATGACCTGTGC |
| | | ATCCTGTGTGTCTAACATAGAAAGGAATCTGCAGAAAGAAGCTGG |
| | | TGTTCTCTCCGTGTTGGTTGCCTTGATGGCAGGAAAGGCAGAGATC |
| | | AAGTATGACCCAGAGGTCATCCAGCCCCTCGAGATAGCTCAGTTC |
| | | ATCCAGGACCTGGGTTTTGAGGCAGCAGTCATGGAGGACTACGCA |
| | | GGCTCCGATGGCAACATTGAGCTGACAATCACAGGGATGACCTGC |
| | | GCGTCCTGTGTCCACAACATAGAGTCCAAACTCACGAGGACAAAT |
| | | GGCATCACTTATGCCTCCGTTGCCCTTGCCACCAGCAAAGCCCTTG |
| | | TTAAGTTTGACCCGGAAATTATCGGTCCACGGGATATTATCAAAAT |
| | | TATTGAGGAAATTGGCTTTCATGCTTCCCTGGCCCAGAGAAACCCC |
| | | AACGCTCATCACTTGGACCACAAGATGGAAATAAAGCAGTGGAAG |
| | | AAGTCTTTCCTGTGCAGCCTGGTGTTTGGCATCCCTGTCATGGCCTT |
| | | AATGATCTATATGCTGATACCCAGCAACGAGCCCCACCAGTCCATG |
| | | GTCCTGGACCACAACATCATTCCAGGACTGTCCATTCTAAATCTCA |
| | | TCTTCTTTATCTTGTGTACCTTTGTCCAGCTCCTCGGTGGGTGGTAC |
| | | TTCTACGTTCAGGCCTACAAATCTCTGAGACACAGGTCAGCCAACA |
| | | TGGACGTGCTCATCGTCCTGGCCACAAGCATTGCTTATGTTTATTCT |
| | | CTGGTCATCCTGGTGGTTGCTGTGGCTGAGAAGGCGGAGAGGAGC |
| | | CCTGTGACATTCTTCGACACGCCCCCCATGCTCTTTGTGTTCATTGC |
| | | CCTGGGCCGGTGGCTGGAACACTTGGCAAAGAGCAAAACCTCAGA |
| | | AGCCCTGGCTAAACTCATGTCTCTCCAAGCCACAGAAGCCACCGTT |
| | | GTGACCCTTGGTGAGGACAATTTAATCATCAGGGAGGAGCAAGTC |
| | | CCCATGGAGCTGGTGCAGCGGGGCGATATCGTCAGGGTGGTCCCT |
| | | GGGGGAAAGTTTCCAGTGGATGGGAAAGTCCTGGAAGGCAATACC |
| | | ATGGCTGATGAGTCCCTCATCACAGGAGAAGCCATGCCAGTCACT |
| | | AAGAAACCCGGAAGCACTGTAATTGCGGGGTCTATAAATGCACAT |
| | | GGCTCTGTGCTCATTAAAGCTACCCACGTGGGCAATGACACCACTT |
| | | TGGCTCAGATTGTGAAACTGGTGGAAGAGGCTCAGATGTCAAAGG |
| | | CACCCATTCAGCAGCTGGCTGACCGGTTTAGTGGATATTTTGTCCC |
| | | ATTTATCATCATCATGTCAACTTTGACGTTGGTGGTATGGATTGTA |
| | | ATCGGTTTTATCGATTTTGGTGTTGTTCAGAAATACTTTCCTAACCC |
| | | CAACAAGCACATCTCCCAGACAGAGGTGATCATCCGGTTTGCTTTC |
| | | CAGACGTCCATCACGGTGCTGTGCATTGCCTGCCCCTGCTCCCTGG |
| | | GGCTGGCCACGCCCACGGCTGTCATGGTGGGCACCGGGGTGGCCG |
| | | CGCAGAACGGCATCCTCATCAAGGGAGGCAAGCCCCTGGAGATGG |
| | | CGCACAAGATAAAGACTGTGATGTTTGACAAGACTGGCACCATTA |
| | | CCCATGGCGTCCCCAGGGTCATGCGGGTGCTCCTGCTGGGGGATGT |
| | | GGCCACACTGCCCCTCAGGAAGGTTCTGGCTGTGGTGGGGACTGC |
| | | GGAGGCCAGCAGTGAACACCCCTTGGGCGTGGCAGTCACCAAATA |
| | | CTGTAAAGAGGAACTTGGAACAGAGACCTTGGGATACTGCACGGA |
| | | CTTCCAGGCAGTGCCAGGCTGTGGAATTGGGTGCAAAGTCAGCAA |
| | | CGTGGAAGGCATCCTGGCCCACAGTGAGCGCCCTTTGAGTGCACC |
| | | GGCCAGTCACCTGAATGAGGCTGGCAGCCTTCCCGCAGAAAAAGA |
| | | TGCAGCCCCCCAGACCTTCTCTGTGCTGATTGGAAACCGTGAGTGG |
| | | CTGAGGCGCAACGGTTTAACCATTTCTAGCGATGTCAGTGACGCTA |
| | | TGACAGACCACGAGATGAAAGGACAGACAGCCATCCTGGTGGCTA |
| | | TTGACGGTGTGCTCTGTGGGATGATCGCAATCGCAGACGCTGTCAA |
| | | GCAGGAGGCTGCCCTGGCTGTGCACACGCTGCAGAGCATGGGTGT |
| | | GGACGTGGTTCTGATCACGGGGGACAACCGGAAGACAGCCAGAGC |
| | | TATTGCCACCCAGGTTGGCATCAACAAAGTCTTTGCAGAGGTGCTG |
| | | CCTTCGCACAAGGTGGCCAAGGTCCAGGAGCTCCAGAATAAAGGG |
| | | AAGAAAGTCGCCATGGTGGGGGATGGGGTCAATGACTCCCCGGCC |
| | | TTGGCCCAGGCAGACATGGGTGTGGCCATTGGCACCGGCACGGAT |
| | | GTGGCCATCGAGGCAGCCGACGTCGTCCTTATCAGAAATGATTTGC |
| | | TGGATGTGGTGGCTAGCATTCACCTTTCCAAGAGGACTGTCCGAAG |
| | | GATACGCATCAACCTGGTCCTGGCACTGATTTATAACCTGGTTGGG |
| | | ATACCCATTGCAGCAGGTGTCTTCATGCCCATCGGCATTGTGCTGC |
| | | AGCCCTGGATGGGCTCAGCGGCCATGGCAGCCTCCTCTGTGTCTGT |
| | | GGTGCTCTCATCCCTGCAGCTCAAGTGCTATAAGAAGCCTGACCTG |
| | | GAGAGGTATGAGGCACAGGCGCATGGCCACATGAAGCCCCTGACG |
| | | GCATCCCAGGTCAGTGTGCACATAGGCATGGATGACAGGTGGCGG |
| | | GACTCCCCCAGGGCCACACCATGGGACCAGGTCAGCTATGTCAGC |
| | | CAGGTGTCGCTGTCCTCCCTGACGTCCGACAAGCCATCTCGGCACA |
| | | GCGCTGCAGCAGACGATGATGGGGACAAGTGGTCTCTGCTCCTGA |
| | | ATGGCAGGGATGAGGAGCAGTACATCTGAAATAAAAGATCTTTAT |
| | | TTTCATTAGATCTGTGTGTTGGTTTTTTGTGTGGATATCGGATCCCG |
| | | TGCGGACCGAGCGGCCGCAGGAACCCCTAGTGATGGAGTTGGCCA |
| | | CTCCCTCTCTGCGCGCTCGCTCGCTCACTGAGGCCGGGCGACCAAA |
| | | GGTCGCCCGACGCCCGGGCTTTGCCCGGGCGGCCTCAGTGAGCGA |
| | | GCGAGCGCGCAGAGAGGGAGTGGCCAA |
| 56 | A3 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAACGCGTCGCCTCGAGGTCAATTCACGCGA |
| | | GTTAATAATTACCAGCGCGGGCCAAATAAATAATCCGCGAGGGGC |
| | | AGGTGACGTTTGCCCAGCGCGCGCTGGTAATTATTAACCTCGCGAA |
| | | TATTGATTCGAGGCCGCGATTGCCGCAATCGCGAGGGGCAGGTGA |
| | | CCTTTGCCCAGCGCGCGTTCGCCCCGCCCCGGACGGTATCGATAAG |
| | | CTTAGGAGCTTGGGCTGCAGGTCGAGGGCACTGGGAGGATGTTGA |
| | | GTAAGATGGAAAACTACTGATGACCCTTGCAGAGACAGAGTATTA |
| | | GGACATGTTTGAACAGGGGCCGGGCGATCAGCAGGTAGCTCTAGA |
| | | GGATCCCCGTCTGTCTGCACATTTCGTAGAGCGAGTGTTCCGATAC |
| | | TCTAATCTCCCTAGGCAAGGTTCATATTTGTGTAGGTTACTTATTCT |
| | | CCTTTTGTTGACTAAGTCAATAATCAGAATCAGCAGGTTTGGAGTC |
| | | AGCTTGGCAGGGATCAGCAGCCTGGGTTGGAAGGAGGGGGTATAA |
| | | AAGCCCCTTCACCAGGAGAAGCCGTCACACAGATCCACAAGCTCC |
| | | TGCGGCCGCCACCATGCCTGAACAGGAGAGACAGATCACAGCCAG |
| | | AGAAGGGGCCAGTCGGAAAATCTTATCTAAGCTTTCTTTGCCTACC |
| | | CGTGCCTGGGAACCAGCAATGAAGAAGAGTTTTGCTTTTGACAAT |
| | | GTTGGCTATGAAGGTGGTCTGGATGGCCTGGGCCCTTCTTCTCAGC |
| | | CGCAGAAGTGCTTCTTACAGATCAAAGGCATGACCTGTGCATCCTG |
| | | TGTGTCTAACATAGAAAGGAATCTGCAGAAAGAAGCTGGTGTTCT |
| | | CTCCGTGTTGGTTGCCTTGATGGCAGGAAAGGCAGAGATCAAGTA |
| | | TGACCCAGAGGTCATCCAGCCCCTCGAGATAGCTCAGTTCATCCAG |
| | | GACCTGGGTTTTGAGGCAGCAGTCATGGAGGACTACGCAGGCTCC |
| | | GATGGCAACATTGAGCTGACAATCACAGGGATGACCTGCGCGTCC |
| | | TGTGTCCACAACATAGAGTCCAAACTCACGAGGACAAATGGCATC |
| | | ACTTATGCCTCCGTTGCCCTTGCCACCAGCAAAGCCCTTGTTAAGT |
| | | TTGACCCGGAAATTATCGGTCCACGGGATATTATCAAAATTATTGA |
| | | GGAAATTGGCTTTCATGCTTCCCTGGCCCAGAGAAACCCCAACGCT |
| | | CATCACTTGGACCACAAGATGGAAATAAAGCAGTGGAAGAAGTCT |
| | | TTCCTGTGCAGCCTGGTGTTTGGCATCCCTGTCATGGCCTTAATGAT |
| | | CTATATGCTGATACCCAGCAACGAGCCCCACCAGTCCATGGTCCTG |
| | | GACCACAACATCATTCCAGGACTGTCCATTCTAAATCTCATCTTCT |
| | | TTATCTTGTGTACCTTTGTCCAGCTCCTCGGTGGGTGGTACTTCTAC |
| | | GTTCAGGCCTACAAATCTCTGAGACACAGGTCAGCCAACATGGAC |
| | | GTGCTCATCGTCCTGGCCACAAGCATTGCTTATGTTTATTCTCTGGT |
| | | CATCCTGGTGGTTGCTGTGGCTGAGAAGGCGGAGAGGAGCCCTGT |
| | | GACATTCTTCGACACGCCCCCCATGCTCTTTGTGTTCATTGCCCTGG |
| | | GCCGGTGGCTGGAACACTTGGCAAAGAGCAAAACCTCAGAAGCCC |
| | | TGGCTAAACTCATGTCTCTCCAAGCCACAGAAGCCACCGTTGTGAC |
| | | CCTTGGTGAGGACAATTTAATCATCAGGGAGGAGCAAGTCCCCAT |
| | | GGAGCTGGTGCAGCGGGGCGATATCGTCAGGGTGGTCCCTGGGGG |
| | | AAAGTTTCCAGTGGATGGGAAAGTCCTGGAAGGCAATACCATGGC |
| | | TGATGAGTCCCTCATCACAGGAGAAGCCATGCCAGTCACTAAGAA |
| | | ACCCGGAAGCACTGTAATTGCGGGGTCTATAAATGCACATGGCTCT |
| | | GTGCTCATTAAAGCTACCCACGTGGGCAATGACACCACTTTGGCTC |
| | | AGATTGTGAAACTGGTGGAAGAGGCTCAGATGTCAAAGGCACCCA |
| | | TTCAGCAGCTGGCTGACCGGTTTAGTGGATATTTTGTCCCATTTATC |
| | | ATCATCATGTCAACTTTGACGTTGGTGGTATGGATTGTAATCGGTT |
| | | TTATCGATTTTGGTGTTGTTCAGAAATACTTTCCTAACCCCAACAA |
| | | GCACATCTCCCAGACAGAGGTGATCATCCGGTTTGCTTTCCAGACG |
| | | TCCATCACGGTGCTGTGCATTGCCTGCCCCTGCTCCCTGGGGCTGG |
| | | CCACGCCCACGGCTGTCATGGTGGGCACCGGGGTGGCCGCGCAGA |
| | | ACGGCATCCTCATCAAGGGAGGCAAGCCCCTGGAGATGGCGCACA |
| | | AGATAAAGACTGTGATGTTTGACAAGACTGGCACCATTACCCATG |
| | | GCGTCCCCAGGGTCATGCGGGTGCTCCTGCTGGGGGATGTGGCCA |
| | | CACTGCCCCTCAGGAAGGTTCTGGCTGTGGTGGGGACTGCGGAGG |
| | | CCAGCAGTGAACACCCCTTGGGCGTGGCAGTCACCAAATACTGTA |
| | | AAGAGGAACTTGGAACAGAGACCTTGGGATACTGCACGGACTTCC |
| | | AGGCAGTGCCAGGCTGTGGAATTGGGTGCAAAGTCAGCAACGTGG |
| | | AAGGCATCCTGGCCCACAGTGAGCGCCCTTTGAGTGCACCGGCCA |
| | | GTCACCTGAATGAGGCTGGCAGCCTTCCCGCAGAAAAAGATGCAG |
| | | CCCCCCAGACCTTCTCTGTGCTGATTGGAAACCGTGAGTGGCTGAG |
| | | GCGCAACGGTTTAACCATTTCTAGCGATGTCAGTGACGCTATGACA |
| | | GACCACGAGATGAAAGGACAGACAGCCATCCTGGTGGCTATTGAC |
| | | GGTGTGCTCTGTGGGATGATCGCAATCGCAGACGCTGTCAAGCAG |
| | | GAGGCTGCCCTGGCTGTGCACACGCTGCAGAGCATGGGTGTGGAC |
| | | GTGGTTCTGATCACGGGGGACAACCGGAAGACAGCCAGAGCTATT |
| | | GCCACCCAGGTTGGCATCAACAAAGTCTTTGCAGAGGTGCTGCCTT |
| | | CGCACAAGGTGGCCAAGGTCCAGGAGCTCCAGAATAAAGGGAAG |
| | | AAAGTCGCCATGGTGGGGGATGGGGTCAATGACTCCCCGGCCTTG |
| | | GCCCAGGCAGACATGGGTGTGGCCATTGGCACCGGCACGGATGTG |
| | | GCCATCGAGGCAGCCGACGTCGTCCTTATCAGAAATGATTTGCTGG |
| | | ATGTGGTGGCTAGCATTCACCTTTCCAAGAGGACTGTCCGAAGGAT |
| | | ACGCATCAACCTGGTCCTGGCACTGATTTATAACCTGGTTGGGATA |
| | | CCCATTGCAGCAGGTGTCTTCATGCCCATCGGCATTGTGCTGCAGC |
| | | CCTGGATGGGCTCAGCGGCCATGGCAGCCTCCTCTGTGTCTGTGGT |
| | | GCTCTCATCCCTGCAGCTCAAGTGCTATAAGAAGCCTGACCTGGAG |
| | | AGGTATGAGGCACAGGCGCATGGCCACATGAAGCCCCTGACGGCA |
| | | TCCCAGGTCAGTGTGCACATAGGCATGGATGACAGGTGGCGGGAC |
| | | TCCCCCAGGGCCACACCATGGGACCAGGTCAGCTATGTCAGCCAG |
| | | GTGTCGCTGTCCTCCCTGACGTCCGACAAGCCATCTCGGCACAGCG |
| | | CTGCAGCAGACGATGATGGGGACAAGTGGTCTCTGCTCCTGAATG |
| | | GCAGGGATGAGGAGCAGTACATCTGAAATAAAAGATCTTTATTTT |
| | | CATTAGATCTGTGTGTTGGTTTTTTGTGTGTCTAGAGTAGATAAGT |
| | | AGCATGGCGGGTTAATCATTAACTACAAGGAACCCCTAGTGATGG |
| | | AGTTGGCCACTCCCTCTCTGCGCGCTCGCTCGCTCACTGAGGCCGG |
| | | GCGACCAAAGGTCGCCCGACGCCCGGGCTTTGCCCGGGCGGCCTC |
| | | AGTGAGCGAGCGAGCGCGCAGAGAGGGAGTGGCCAA |
| 57 | A4 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAACGCGTCGCCTCGAGGTCAATTCACGCGA |
| | | GTTAATAATTACCAGCGCGGGCCAAATAAATAATCCGCGAGGGGC |
| | | AGGTGACGTTTGCCCAGCGCGCGCTGGTAATTATTAACCTCGCGAA |
| | | TATTGATTCGAGGCCGCGATTGCCGCAATCGCGAGGGGCAGGTGA |
| | | CCTTTGCCCAGCGCGCGTTCGCCCCGCCCCGGACGGTATCGATAAG |
| | | CTTAGGAGCTTGGGCTGCAGGTCGAGGGCACTGGGAGGATGTTGA |
| | | GTAAGATGGAAAACTACTGATGACCCTTGCAGAGACAGAGTATTA |
| | | GGACATGTTTGAACAGGGGCCGGGCGATCAGCAGGTAGCTCTAGA |
| | | GGATCCCCGTCTGTCTGCACATTTCGTAGAGCGAGTGTTCCGATAC |
| | | TCTAATCTCCCTAGGCAAGGTTCATATTTGTGTAGGTTACTTATTCT |
| | | CCTTTTGTTGACTAAGTCAATAATCAGAATCAGCAGGTTTGGAGTC |
| | | AGCTTGGCAGGGATCAGCAGCCTGGGTTGGAAGGAGGGGGTATAA |
| | | AAGCCCCTTCACCAGGAGAAGCCGTCACACAGATCCACAAGCTCC |
| | | TGCGCGGCCGCAAGATGGCGGCGCCTGAGCAGGAGAGACAGATCA |
| | | CAGCCAGAGAAGGGGCCAGTCGGAAAATCTTATCTAAGCTTTCTTT |
| | | GCCTACCCGTGCCTGGGAACCAGCAATGAAGAAGAGTTTTGCTTTT |
| | | GACAATGTTGGCTATGAAGGTGGTCTGGATGGCCTGGGCCCTTCTT |
| | | CTCAGGTGGCCCCTGATGGAGCCGAAGGGAGTGGGACAGATCACA |
| | | GGTCTTCCAGTTCTCATTCCCCTGGCTCCCCACCGAGAAACCAGGT |
| | | CCAGGGCACATGCAGTAAGTGCTTCTTACAGATCAAAGGCATGAC |
| | | CTGTGCATCCTGTGTGTCTAACATAGAAAGGAATCTGCAGAAAGA |
| | | AGCTGGTGTTCTCTCCGTGTTGGTTGCCTTGATGGCAGGAAAGGCA |
| | | GAGATCAAGTATGACCCAGAGGTCATCCAGCCCCTCGAGATAGCT |
| | | CAGTTCATCCAGGACCTGGGTTTTGAGGCAGCAGTCATGGAGGAC |
| | | TACGCAGGCTCCGATGGCAACATTGAGCTGACAATCACAGGGATG |
| | | ACCTGCGCGTCCTGTGTCCACAACATAGAGTCCAAACTCACGAGG |
| | | ACAAATGGCATCACTTATGCCTCCGTTGCCCTTGCCACCAGCAAAG |
| | | CCCTTGTTAAGTTTGACCCGGAAATTATCGGTCCACGGGATATTAT |
| | | CAAAATTATTGAGGAAATTGGCTTTCATGCTTCCCTGGCCCAGAGA |
| | | AACCCCAACGCTCATCACTTGGACCACAAGATGGAAATAAAGCAG |
| | | TGGAAGAAGTCTTTTCTTGTGAGTCTGTTTTTCTGTATTCCTGTAAT |
| | | GGGGCTGATGATATATATGATGGTTATGGACCACCACTTTGCAACT |
| | | CTTCACCATAATCAAAACATGAGTAAAGAAGAAATGATCAACCTT |
| | | CATTCTTCTATGTTCCTGGAGCGCCAGATTCTTCCAGGATTGTCTGT |
| | | TATGAATTTGCTGTCCTTTTTATTGTGTGTACCTGTACAGTTTTTCG |
| | | GAGGCTGGTACTTCTACATTCAGGCTTATAAAGCACTGAAGCATAA |
| | | GACAGCAAATATGGACGTACTGATTGTGCTGGCAACCACCATTGC |
| | | ATTTGCCTACTCTTTGATTATTCTTCTAGTTGCAATGTATGAGAGAG |
| | | CCAAAGTGAACCCTATTACTTTCTTTGACACACCCCCTATGCTGTTT |
| | | GTGTTTATTGCACTAGGCCGATGGCTGGAACATATAGCAAAGGGC |
| | | AAAACATCAGAGGCTCTTGCAAAGTTAATTTCACTACAAGCTACA |
| | | GAAGCAACTATTGTAACTCTTGATTCTGATAATATCCTCCTCAGTG |
| | | AAGAACAAGTGGATGTGGAACTTGTACAACGTGGAGATATCATTA |
| | | AAGTAGTTCCAGGAGGCAAATTTCCAGTGGATGGTCGTGTTATTGA |
| | | AGGACATTCTATGGTAGATGAGTCCCTCATCACAGGGGAGGCAAT |
| | | GCCTGTGGCTAAGAAACCTGGCAGCACAGTGATTGCTGGTTCCATT |
| | | AACCAGAACGGGTCACTGCTTATCTGCGCAACACATGTTGGAGCA |
| | | GACACAACCCTTTCTCAAATTGTCAAACTTGTGGAAGAGGCACAA |
| | | ACATCAAAGGCTCCTATCCAGCAGTTTGCAGACAAACTCAGTGGCT |
| | | ATTTTGTTCCTTTTATTGTTTTTGTTTCCATTGCCACCCTCTTGGTAT |
| | | GGATTGTAATTGGATTTCTGAATTTTGAAATTGTGGAAACCTACTT |
| | | TCCTGGCTACAATAGAAGTATCTCCCGAACAGAAACGATAATACG |
| | | ATTTGCTTTCCAAGCCTCTATCACAGTTCTGTGTATTGCATGTCCCT |
| | | GTTCACTGGGACTGGCCACTCCAACTGCTGTGATGGTGGGTACAGG |
| | | AGTAGGTGCTCAAAATGGCATACTAATAAAAGGTGGAGAGCCATT |
| | | GGAGATGGCTCATAAGGTAAAGGTAGTGGTATTTGATAAGACTGG |
| | | AACCATTACTCACGGAACCCCAGTGGTGAATCAAGTAAAGGTTCT |
| | | AACTGAAAGTAACAGAATATCACACCATAAAATCTTGGCCATTGT |
| | | GGGAACTGCTGAAAGTAACAGTGAACACCCTCTAGGAACAGCCAT |
| | | AACCAAATATTGCAAACAGGAGCTGGACACTGAAACCTTGGGTAC |
| | | CTGCATAGATTTCCAGGTTGTGCCAGGCTGTGGTATTAGCTGTAAA |
| | | GTCACCAATATTGAAGGCTTGCTACATAAGAATAACTGGAATATA |
| | | GAGGACAATAATATTAAAAATGCATCCCTGGTTCAAATTGATGCC |
| | | AGTAATGAACAGTCATCAACTTCGTCTTCCATGATTATTGATGCCC |
| | | AGATCTCAAATGCTCTTAATGCTCAGCAGTATAAAGTCCTCATTGG |
| | | TAACCGGGAGTGGATGATTAGAAATGGTCTTGTCATTAATAACGAT |
| | | GTAAATGATTTCATGACTGAACATGAGAGAAAAGGTCGGACTGCT |
| | | GTATTAGTAGCAGTTGATGATGAGCTGTGTGGCTTGATAGCCATTG |
| | | CAGACACAGTGAAGCCTGAAGCAGAACTGGCTATCCATATTCTGA |
| | | AATCTATGGGCTTAGAAGTAGTTCTGATGACTGGAGACAACAGTA |
| | | AAACAGCTAGATCTATTGCTTCTCAGGTTGGCATTACTAAGGTGTT |
| | | TGCTGAAGTTCTACCTTCTCACAAGGTTGCTAAAGTGAAGCAACTT |
| | | CAAGAGGAGGGGAAACGGGTAGCAATGGTGGGAGATGGAATCAA |
| | | TGACTCCCCAGCTCTGGCAATGGCTAATGTGGGAATTGCTATTGGC |
| | | ACAGGCACAGATGTAGCCATTGAAGCAGCTGATGTGGTTTTGATA |
| | | AGGAATGATCTTCTGGATGTAGTGGCAAGTATTGACTTATCAAGAA |
| | | AGACAGTCAAGAGGATTCGGATAAATTTTGTCTTTGCTCTAATTTA |
| | | TAATCTGGTTGGAATTCCCATAGCTGCTGGAGTTTTTATGCCCATT |
| | | GGTTTGGTTTTGCAGCCCTGGATGGGATCTGCAGCAATGGCTGCTT |
| | | CATCTGTTTCTGTAGTACTTTCTTCTCTCCAGCTCAAGTGCTATAAG |
| | | AAGCCTGACCTGGAGAGGTATGAGGCACAGGCGCATGGCCACATG |
| | | AAGCCCCTGACGGCATCCCAGGTCAGTGTGCACATAGGCATGGAT |
| | | GACAGGTGGCGGGACTCCCCCAGGGCCACACCATGGGACCAGGTC |
| | | AGCTATGTCAGCCAGGTGTCGCTGTCCTCCCTGACGTCCGACAAGC |
| | | CATCTCGGCACAGCGCTGCAGCAGACGATGATGGGGACAAGTGGT |
| | | CTCTGCTCCTGAATGGCAGGGATGAGGAGCAGTACATCTGAGAAT |
| | | TCGATATCAAGCTTATCGATAATCAACCTCTGGATTACAAAATTTG |
| | | TGAAAGATTGACTGGTATTCTTAACTATGTTGCTCCTTTTACGCTAT |
| | | GTGGATACGCTGCTTTAATGCCTTTGTATCATGCTATTGCTTCCCGT |
| | | ATGGCTTTCATTTTCTCCTCCTTGTATAAATCCTGGTTGCTGTCTCT |
| | | TTATGAGGAGTTGTGGCCCGTTGTCAGGCAACGTGGCGTGGTGTGC |
| | | ACTGTGTTTGCTGACGCAACCCCCACTGGTTGGGGCACCTGCAGGA |
| | | ATAAAAGATCTTTATTTTCATTAGATCTGTGTGTTGGTTTTTTGTGT |
| | | GTCTAGAGTAGATAAGTAGCATGGCGGGTTAATCATTAACTACAA |
| | | GGAACCCCTAGTGATGGAGTTGGCCACTCCCTCTCTGCGCGCTCGC |
| | | TCGCTCACTGAGGCCGGGCGACCAAAGGTCGCCCGACGCCCGGGC |
| | | TTTGCCCGGGCGGCCTCAGTGAGCGAGCGAGCGCGCAGAGAGGGA |
| | | GTGGCCAA |
| 58 | A5 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAACGCGTCGCCTCGAGGTCAATTCACGCGA |
| | | GTTAATAATTACCAGCGCGGGCCAAATAAATAATCCGCGAGGGGC |
| | | AGGTGACGTTTGCCCAGCGCGCGCTGGTAATTATTAACCTCGCGAA |
| | | TATTGATTCGAGGCCGCGATTGCCGCAATCGCGAGGGGCAGGTGA |
| | | CCTTTGCCCAGCGCGCGTTCGCCCCGCCCCGGACGGTATCGATAAG |
| | | CTTAGGAGCTTGGGCTGCAGGTCGAGGGCACTGGGAGGATGTTGA |
| | | GTAAGATGGAAAACTACTGATGACCCTTGCAGAGACAGAGTATTA |
| | | GGACATGTTTGAACAGGGGCCGGGCGATCAGCAGGTAGCTCTAGA |
| | | GGATCCCCGTCTGTCTGCACATTTCGTAGAGCGAGTGTTCCGATAC |
| | | TCTAATCTCCCTAGGCAAGGTTCATATTTGTGTAGGTTACTTATTCT |
| | | CCTTTTGTTGACTAAGTCAATAATCAGAATCAGCAGGTTTGGAGTC |
| | | AGCTTGGCAGGGATCAGCAGCCTGGGTTGGAAGGAGGGGGTATAA |
| | | AAGCCCCTTCACCAGGAGAAGCCGTCACACAGATCCACAAGCTCC |
| | | TGCGGCCGCCACCATGCCTGAGCAGGAGAGACAGATCACAGCCAG |
| | | AGAAGGGGCCAGTCGGAAAATCTTATCTAAGCTTTCTTTGCCTACC |
| | | CGTGCCTGGGAACCAGCAATGAAGAAGAGTTTTGCTTTTGACAAT |
| | | GTTGGCTATGAAGGTGGTCTGGATGGCCTGGGCCCTTCTTCTCAGG |
| | | TGGCCACCACTCTGATTGCCATTGCCGGCATGACCTGTGCATCCTG |
| | | TGTCCATTCCATTGAAGGCATGATCTCCCAACTGGAAGGGGTGCAG |
| | | CAAATATCGGTGTCTTTGGCCGAAGGGACTGCAACAGTTCTTTATA |
| | | ATCCCTCTGTAATTAGCCCAGAAGAACTCAGAGCTGCTATAGAAG |
| | | ACATGGGATTTGAGGCTTCAGTCGTTTCTGAAAGCTGTTCTACTAA |
| | | CCCTCTTGGAAACCACAGTGCTGGGAATTCCATGGTGCAAACTACA |
| | | GATGGTACACCTACATCTGTGCAGGAAGTGGCTCCCCACACTGGG |
| | | AGGCTCCCTGCAAACCATGCCCCGGACATCTTGGCAAAGTCCCCAC |
| | | AATCAACCAGAGCAGTGGCACCGCAGAAGTGCTTCTTACAGATCA |
| | | AAGGCATGACCTGTGCATCCTGTGTGTCTAACATAGAAAGGAATCT |
| | | GCAGAAAGAAGCTGGTGTTCTCTCCGTGTTGGTTGCCTTGATGGCA |
| | | GGAAAGGCAGAGATCAAGTATGACCCAGAGGTCATCCAGCCCCTC |
| | | GAGATAGCTCAGTTCATCCAGGACCTGGGTTTTGAGGCAGCAGTC |
| | | ATGGAGGACTACGCAGGCTCCGATGGCAACATTGAGCTGACAATC |
| | | ACAGGGATGACCTGCGCGTCCTGTGTCCACAACATAGAGTCCAAA |
| | | CTCACGAGGACAAATGGCATCACTTATGCCTCCGTTGCCCTTGCCA |
| | | CCAGCAAAGCCCTTGTTAAGTTTGACCCGGAAATTATCGGTCCACG |
| | | GGATATTATCAAAATTATTGAGGAAATTGGCTTTCATGCTTCCCTG |
| | | GCCCAGAGAAACCCCAACGCTCATCACTTGGACCACAAGATGGAA |
| | | ATAAAGCAGTGGAAGAAGTCTTTCCTGTGCAGCCTGGTGTTTGGCA |
| | | TCCCTGTCATGGCCTTAATGATCTATATGCTGATACCCAGCAACGA |
| | | GCCCCACCAGTCCATGGTCCTGGACCACAACATCATTCCAGGACTG |
| | | TCCATTCTAAATCTCATCTTCTTTATCTTGTGTACCTTTGTCCAGCT |
| | | CCTCGGTGGGTGGTACTTCTACGTTCAGGCCTACAAATCTCTGAGA |
| | | CACAGGTCAGCCAACATGGACGTGCTCATCGTCCTGGCCACAAGC |
| | | ATTGCTTATGTTTATTCTCTGGTCATCCTGGTGGTTGCTGTGGCTGA |
| | | GAAGGCGGAGAGGAGCCCTGTGACATTCTTCGACACGCCCCCCAT |
| | | GCTCTTTGTGTTCATTGCCCTGGGCCGGTGGCTGGAACACTTGGCA |
| | | AAGAGCAAAACCTCAGAAGCCCTGGCTAAACTCATGTCTCTCCAA |
| | | GCCACAGAAGCCACCGTTGTGACCCTTGGTGAGGACAATTTAATC |
| | | ATCAGGGAGGAGCAAGTCCCCATGGAGCTGGTGCAGCGGGGCGAT |
| | | ATCGTCAAGGTGGTCCCTGGGGGAAAGTTTCCAGTGGATGGGAAA |
| | | GTCCTGGAAGGCAATACCATGGCTGATGAGTCCCTCATCACAGGA |
| | | GAAGCCATGCCAGTCACTAAGAAACCCGGAAGCACTGTAATTGCG |
| | | GGGTCTATAAATGCACATGGCTCTGTGCTCATTAAAGCTACCCACG |
| | | TGGGCAATGACACCACTTTGGCTCAGATTGTGAAACTGGTGGAAG |
| | | AGGCTCAGATGTCAAAGGCACCCATTCAGCAGCTGGCTGACCGGT |
| | | TTAGTGGATATTTTGTCCCATTTATCATCATCATGTCAACTTTGACG |
| | | TTGGTGGTATGGATTGTAATCGGTTTTATCGATTTTGGTGTTGTTCA |
| | | GAGATACTTTCCTAACCCCAACAAGCACATCTCCCAGACAGAGGT |
| | | GATCATCCGGTTTGCTTTCCAGACGTCCATCACGGTGCTGTGCATT |
| | | GCCTGCCCCTGCTCCCTGGGGCTGGCCACGCCCACGGCTGTCATGG |
| | | TGGGCACCGGGGTGGCCGCGCAGAACGGCATCCTCATCAAGGGAG |
| | | GCAAGCCCCTGGAGATGGCGCACAAGATAAAGACTGTGATGTTTG |
| | | ACAAGACTGGCACCATTACCCATGGCGTCCCCAGGGTCATGCGGG |
| | | TGCTCCTGCTGGGGGATGTGGCCACACTGCCCCTCAGGAAGGTTCT |
| | | GGCTGTGGTGGGGACTGCGGAGGCCAGCAGTGAACACCCCTTGGG |
| | | CGTGGCAGTCACCAAATACTGTAAAGAGGAACTTGGAACAGAGAC |
| | | CTTGGGATACTGCACGGACTTCCAGGCAGTGCCAGGCTGTGGAATT |
| | | GGGTGCAAAGTCAGCAACGTGGAAGGCATCCTGGCCCACAGTGAG |
| | | CGCCCTTTGAGTGCACCGGCCAGTCACCTGAATGAGGCTGGCAGC |
| | | CTTCCCGCAGAAAAAGATGCAGTCCCCCAGACCTTCTCTGTGCTGA |
| | | TTGGAAACCGTGAGTGGCTGAGGCGCAACGGTTTAACCATTTCTAG |
| | | CGATGTCAGTGACGCTATGACAGACCACGAGATGAAAGGACAGAC |
| | | AGCCATCCTGGTGGCTATTGACGGTGTGCTCTGTGGGATGATCGCA |
| | | ATCGCAGACGCTGTCAAGCAGGAGGCTGCCCTGGCTGTGCACACG |
| | | CTGCAGAGCATGGGTGTGGACGTGGTTCTGATCACGGGGGACAAC |
| | | CGGAAGACAGCCAGAGCTATTGCCACCCAGGTTGGCATCAACAAA |
| | | GTCTTTGCAGAGGTGCTGCCTTCGCACAAGGTGGCCAAGGTCCAG |
| | | GAGCTCCAGAATAAAGGGAAGAAAGTCGCCATGGTGGGGGATGG |
| | | GGTCAATGACTCCCCGGCCTTGGCCCAGGCAGACATGGGTGTGGC |
| | | CATTGGCACCGGCACGGATGTGGCCATCGAGGCAGCCGACGTCGT |
| | | CCTTATCAGAAATGATTTGCTGGATGTGGTGGCTAGCATTCACCTT |
| | | TCCAAGAGGACTGTCCGAAGGATACGCATCAACCTGGTCCTGGCA |
| | | CTGATTTATAACCTGGTTGGGATACCCATTGCAGCAGGTGTCTTCA |
| | | TGCCCATCGGCATTGTGCTGCAGCCCTGGATGGGCTCAGCGGCCAT |
| | | GGCAGCCTCCTCTGTGTCTGTGGTGCTCTCATCCCTGCAGCTCAAG |
| | | TGCTATAAGAAGCCTGACCTGGAGAGGTATGAGGCACAGGCGCAT |
| | | GGCCACATGAAGCCCCTGACGGCATCCCAGGTCAGTGTGCACATA |
| | | GGCATGGATGACAGGTGGCGGGACTCCCCCAGGGCCACACCATGG |
| | | GACCAGGTCAGCTATGTCAGCCAGGTGTCGCTGTCCTCCCTGACGT |
| | | CCGACAAGCCATCTCGGCACAGCGCTGCAGCAGACGATGATGGGG |
| | | ACAAGTGGTCTCTGCTCCTGAATGGCAGGGATGAGGAGCAGTACA |
| | | TCTGAAATAAAAGATCTTTATTTTCATTAGATCTGTGTGTTGGTTTT |
| | | TTGTGTGTCTAGAGTAGATAAGTAGCATGGCGGGTTAATCATTAAC |
| | | TACAAGGAACCCCTAGTGATGGAGTTGGCCACTCCCTCTCTGCGCG |
| | | CTCGCTCGCTCACTGAGGCCGGGCGACCAAAGGTCGCCCGACGCC |
| | | CGGGCTTTGCCCGGGCGGCCTCAGTGAGCGAGCGAGCGCGCAGAG |
| | | AGGGAGTGGCCAA |
| 59 | A6 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAACGCGTCGCCTCGAGGTCAATTCACGCGA |
| | | GTTAATAATTACCAGCGCGGGCCAAATAAATAATCCGCGAGGGGC |
| | | AGGTGACGTTTGCCCAGCGCGCGCTGGTAATTATTAACCTCGCGAA |
| | | TATTGATTCGAGGCCGCGATTGCCGCAATCGCGAGGGGCAGGTGA |
| | | CCTTTGCCCAGCGCGCGTTCGCCCCGCCCCGGACGGTATCGATAAG |
| | | CTTAGGAGCTTGGGCTGCAGGTCGAGGGCACTGGGAGGATGTTGA |
| | | GTAAGATGGAAAACTACTGATGACCCTTGCAGAGACAGAGTATTA |
| | | GGACATGTTTGAACAGGGGCCGGGCGATCAGCAGGTAGCTCTAGA |
| | | GGATCCCCGTCTGTCTGCACATTTCGTAGAGCGAGTGTTCCGATAC |
| | | TCTAATCTCCCTAGGCAAGGTTCATATTTGTGTAGGTTACTTATTCT |
| | | CCTTTTGTTGACTAAGTCAATAATCAGAATCAGCAGGTTTGGAGTC |
| | | AGCTTGGCAGGGATCAGCAGCCTGGGTTGGAAGGAGGGGGTATAA |
| | | AAGCCCCTTCACCAGGAGAAGCCGTCACACAGATCCACAAGCTCC |
| | | TGCGGCCGCCACCATGCCGGAACAAGAACGCCAGATCACAGCAAG |
| | | AGAAGGAGCAAGTAGGAAGATCCTGAGCAAGCTCTCCCTGCCCAC |
| | | AAGGGCCTGGGAGCCGGCCATGAAGAAGTCTTTTGCCTTCGATAA |
| | | TGTCGGTTATGAAGGCGGGCTCGATGGCCTGGGCCCCAGCAGCCA |
| | | GGTGGCGACCACCTTAATCGCCATCGCAGGCATGACCTGCGCATCT |
| | | TGTGTGCATAGCATTGAAGGCATGATTAGCCAGCTGGAGGGCGTG |
| | | CAGCAGATCTCAGTCTCCCTGGCTGAGGGGACCGCCACTGTCTTGT |
| | | ACAATCCGTCTGTTATTTCCCCGGAGGAGCTGAGAGCTGCCATTGA |
| | | GGACATGGGATTTGAAGCATCGGTGGTGTCTGAGTCATGCTCCACC |
| | | AATCCCCTGGGAAATCACTCAGCTGGCAATTCAATGGTTCAGACCA |
| | | CCGATGGGACCCCAACCAGCGTGCAGGAAGTGGCACCCCATACCG |
| | | GCAGACTGCCTGCCAATCATGCTCCTGATATCCTCGCCAAGAGCCC |
| | | CCAGAGCACCCGCGCGGTGGCACCCCAGAAGTGTTTCCTGCAGAT |
| | | CAAGGGCATGACCTGTGCCAGCTGTGTTAGCAATATCGAAAGAAA |
| | | CCTCCAAAAAGAGGCTGGAGTTCTGAGTGTTCTAGTGGCTTTAATG |
| | | GCCGGGAAAGCCGAGATCAAGTATGATCCCGAGGTGATTCAGCCA |
| | | TTGGAGATTGCCCAGTTCATCCAGGATCTGGGCTTCGAGGCCGCCG |
| | | TGATGGAGGATTACGCCGGCTCTGATGGCAACATCGAACTGACCA |
| | | TCACCGGCATGACCTGTGCTTCCTGCGTGCATAATATCGAGAGCAA |
| | | ACTGACGCGGACCAATGGAATCACCTATGCCAGCGTCGCCCTGGC |
| | | AACCTCTAAGGCTCTGGTGAAGTTTGACCCCGAAATCATTGGACCT |
| | | AGAGACATCATCAAAATTATTGAAGAGATTGGTTTCCACGCCAGCT |
| | | TAGCTCAGCGGAATCCCAACGCACACCATCTTGACCATAAGATGG |
| | | AAATCAAACAGTGGAAGAAGAGCTTCTTGTGTAGCCTGGTCTTCG |
| | | GGATACCCGTCATGGCACTGATGATTTACATGCTCATTCCGTCCAA |
| | | CGAGCCCCACCAGTCCATGGTGCTGGACCACAACATCATCCCTGG |
| | | GCTGTCTATCCTCAACCTGATCTTCTTTATTCTGTGCACATTTGTCC |
| | | AGCTCTTGGGAGGCTGGTATTTCTATGTACAGGCCTACAAATCCCT |
| | | GAGGCATCGAAGCGCCAACATGGACGTTTTAATTGTGCTGGCTAC |
| | | ATCCATTGCCTACGTGTACTCCCTGGTGATCTTAGTGGTTGCTGTG |
| | | GCTGAAAAGGCTGAGAGAAGCCCTGTCACCTTCTTCGACACCCCCC |
| | | CCATGCTCTTCGTGTTTATTGCACTGGGTCGCTGGCTGGAGCATTT |
| | | AGCCAAGTCCAAGACATCCGAGGCTCTGGCCAAGCTGATGTCTCT |
| | | ACAGGCCACGGAGGCCACCGTGGTGACTCTGGGTGAAGACAATTT |
| | | GATAATACGCGAGGAACAGGTGCCTATGGAGCTTGTGCAGCGCGG |
| | | AGATATCGTCAAGGTGGTGCCCGGCGGCAAGTTCCCAGTGGATGG |
| | | AAAAGTGTTAGAGGGCAATACCATGGCCGATGAATCCCTCATCAC |
| | | TGGCGAGGCAATGCCGGTGACCAAGAAACCTGGAAGTACTGTCAT |
| | | TGCAGGCAGCATCAATGCCCATGGGAGCGTCCTGATCAAGGCCAC |
| | | CCACGTGGGCAATGATACAACTCTCGCCCAGATCGTCAAGCTAGT |
| | | GGAAGAGGCTCAGATGTCTAAAGCTCCAATCCAGCAACTGGCTGA |
| | | CCGCTTCAGCGGCTACTTTGTCCCCTTTATCATCATCATGAGCACCC |
| | | TCACCCTGGTGGTGTGGATAGTCATAGGCTTCATTGACTTTGGCGT |
| | | GGTGCAGAGATACTTCCCCAACCCTAACAAACATATCTCCCAAACC |
| | | GAGGTCATTATCAGATTCGCCTTTCAGACATCTATCACTGTGCTGT |
| | | GCATAGCCTGCCCTTGCTCTCTGGGCCTGGCCACCCCCACCGCCGT |
| | | GATGGTTGGAACCGGGGTCGCTGCACAAAATGGGATCCTGATAAA |
| | | AGGGGGCAAACCCCTGGAAATGGCACATAAGATTAAAACAGTTAT |
| | | GTTTGATAAGACAGGCACCATTACTCACGGTGTCCCTCGAGTCATG |
| | | AGAGTGCTGCTGCTGGGGGACGTGGCTACACTGCCACTGCGCAAG |
| | | GTGCTGGCTGTGGTGGGCACAGCGGAGGCCTCCTCAGAACACCCA |
| | | TTAGGCGTAGCTGTCACAAAATACTGTAAGGAGGAGCTGGGGACA |
| | | GAGACCCTCGGCTATTGTACAGACTTCCAGGCCGTGCCTGGCTGCG |
| | | GCATCGGCTGTAAGGTCAGCAATGTGGAGGGCATCCTGGCACACA |
| | | GCGAGAGACCCCTCTCTGCCCCAGCCTCCCACCTGAATGAAGCTGG |
| | | GTCTCTGCCTGCTGAGAAGGACGCCGTCCCTCAGACCTTCTCCGTG |
| | | TTGATTGGGAACAGAGAGTGGCTTAGAAGGAATGGCCTGACCATC |
| | | AGCTCTGACGTCTCGGATGCCATGACAGATCACGAGATGAAAGGC |
| | | CAGACAGCCATACTGGTTGCCATCGACGGCGTGCTCTGTGGCATGA |
| | | TTGCCATCGCTGATGCTGTTAAACAGGAAGCTGCGCTGGCAGTCCA |
| | | CACCCTCCAGTCCATGGGAGTGGACGTGGTCCTGATTACTGGAGAC |
| | | AACAGGAAAACCGCTCGGGCCATCGCTACCCAAGTGGGGATAAAT |
| | | AAGGTTTTTGCAGAAGTCTTACCCTCCCACAAAGTAGCCAAAGTGC |
| | | AGGAGCTCCAGAACAAGGGTAAAAAGGTGGCAATGGTGGGAGAT |
| | | GGTGTGAATGACAGCCCAGCTCTCGCGCAGGCTGACATGGGCGTA |
| | | GCCATAGGTACTGGAACAGATGTCGCGATTGAAGCCGCTGACGTA |
| | | GTCCTCATCCGAAACGATTTGCTGGATGTGGTGGCCAGCATCCATC |
| | | TGTCCAAGCGTACCGTGCGCCGGATCCGCATCAACCTGGTCTTGGC |
| | | CCTCATCTATAATCTCGTGGGCATCCCCATCGCTGCTGGGGTCTTC |
| | | ATGCCCATCGGCATTGTGCTGCAGCCCTGGATGGGAAGCGCTGCC |
| | | ATGGCTGCTTCTTCTGTCTCCGTTGTGCTGTCCAGCCTACAGCTGAA |
| | | GTGCTATAAAAAGCCAGACCTCGAGCGCTACGAAGCCCAGGCGCA |
| | | TGGCCATATGAAGCCCCTCACGGCTTCCCAGGTCTCGGTTCACATT |
| | | GGGATGGATGACAGATGGCGGGACTCCCCACGCGCCACACCCTGG |
| | | GACCAGGTATCCTATGTGAGCCAGGTGAGCCTCAGCTCCCTGACAT |
| | | CAGATAAACCGTCTAGGCACTCAGCTGCCGCAGATGATGACGGTG |
| | | ACAAATGGAGCCTCCTCCTGAACGGCCGCGACGAGGAGCAGTACA |
| | | TCTAAAATAAAAGATCTTTATTTTCATTAGATCTGTGTGTTGGTTTT |
| | | TTGTGTGTCTAGAGTAGATAAGTAGCATGGCGGGTTAATCATTAAC |
| | | TACAAGGAACCCCTAGTGATGGAGTTGGCCACTCCCTCTCTGCGCG |
| | | CTCGCTCGCTCACTGAGGCCGGGCGACCAAAGGTCGCCCGACGCC |
| | | CGGGCTTTGCCCGGGCGGCCTCAGTGAGCGAGCGAGCGCGCAGAG |
| | | AGGGAGTGGCCAA |
| 60 | A7 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAACGCGTCGCCTCGAGGTCAATTCACGCGA |
| | | GTTAATAATTACCAGCGCGGGCCAAATAAATAATCCGCGAGGGGC |
| | | AGGTGACGTTTGCCCAGCGCGCGCTGGTAATTATTAACCTCGCGAA |
| | | TATTGATTCGAGGCCGCGATTGCCGCAATCGCGAGGGGCAGGTGA |
| | | CCTTTGCCCAGCGCGCGTTCGCCCCGCCCCGGACGGTATCGATAAG |
| | | CTTAGGAGCTTGGGCTGCAGGTCGAGGGCACTGGGAGGATGTTGA |
| | | GTAAGATGGAAAACTACTGATGACCCTTGCAGAGACAGAGTATTA |
| | | GGACATGTTTGAACAGGGGCCGGGCGATCAGCAGGTAGCTCTAGA |
| | | GGATCCCCGTCTGTCTGCACATTTCGTAGAGCGAGTGTTCCGATAC |
| | | TCTAATCTCCCTAGGCAAGGTTCATATTTGTGTAGGTTACTTATTCT |
| | | CCTTTTGTTGACTAAGTCAATAATCAGAATCAGCAGGTTTGGAGTC |
| | | AGCTTGGCAGGGATCAGCAGCCTGGGTTGGAAGGAGGGGGTATAA |
| | | AAGCCCCTTCACCAGGAGAAGCCGTCACACAGATCCACAAGCTCC |
| | | TGCGGCCGCCACCATGCCTGAGCAGGAGAGACAGATCACAGCCAG |
| | | AGAAGGGGCCAGTCGGAAAATCTTATCTAAGCTTTCTTTGCCTACC |
| | | CGTGCCTGGGAACCAGCAATGAAGAAGAGTTTTGCTTTTGACAAT |
| | | GTTGGCTATGAAGGTGGTCTGGATGGCCTGGGCCCTTCTTCTCAGG |
| | | TGGCCCCTGATGGAGCCGAAGGGAGTGGGACAGATCACAGGTCTT |
| | | CCAGTTCTCATTCCCCTGGCTCCCCACCGAGAAACCAGGTCCAGGG |
| | | CACATGCAGTACCACTCTGATTGCCATTGCCGGCATGACCTGTGCA |
| | | TCCTGTGTCCATTCCATTGAAGGCATGATCTCCCAACTGGAAGGGG |
| | | TGCAGCAAATATCGGTGTCTTTGGCCGAAGGGACTGCAACAGTTCT |
| | | TTATAATCCCTCTGTAATTAGCCCAGAAGAACTCAGAGCTGCTATA |
| | | GAAGACATGGGATTTGAGGCTTCAGTCGTTTCTGAAAGCTGTTCTA |
| | | CTAACCCTCTTGGAAACCACAGTGCTGGGAATTCCATGGTGCAAAC |
| | | TACAGATGGTACACCTACATCTGTGCAGGAAGTGGCTCCCCACACT |
| | | GGGAGGCTCCCTGCAAACCATGCCCCGGACATCTTGGCAAAGTCC |
| | | CCACAATCAACCAGAGCAGTGGCACCGCAGAAGTGCTTCTTACAG |
| | | ATCAAAGGCATGACCTGTGCATCCTGTGTGTCTAACATAGAAAGG |
| | | AATCTGCAGAAAGAAGCTGGTGTTCTCTCCGTGTTGGTTGCCTTGA |
| | | TGGCAGGAAAGGCAGAGATCAAGTATGACCCAGAGGTCATCCAGC |
| | | CCCTCGAGATAGCTCAGTTCATCCAGGACCTGGGTTTTGAGGCAGC |
| | | AGTCATGGAGGACTACGCAGGCTCCGATGGCAACATTGAGCTGAC |
| | | AATCACAGGGATGACCTGCGCGTCCTGTGTCCACAACATAGAGTC |
| | | CAAACTCACGAGGACAAATGGCATCACTTATGCCTCCGTTGCCCTT |
| | | GCCACCAGCAAAGCCCTTGTTAAGTTTGACCCGGAAATTATCGGTC |
| | | CACGGGATATTATCAAAATTATTGAGGAAATTGGCTTTCATGCTTC |
| | | CCTGGCCCAGAGAAACCCCAACGCTCATCACTTGGACCACAAGAT |
| | | GGAAATAAAGCAGTGGAAGAAGTCTTTCCTGTGCAGCCTGGTGTTT |
| | | GGCATCCCTGTCATGGCCTTAATGATCTATATGCTGATACCCAGCA |
| | | ACGAGCCCCACCAGTCCATGGTCCTGGACCACAACATCATTCCAG |
| | | GACTGTCCATTCTAAATCTCATCTTCTTTATCTTGTGTACCTTTGTC |
| | | CAGCTCCTCGGTGGGTGGTACTTCTACGTTCAGGCCTACAAATCTC |
| | | TGAGACACAGGTCAGCCAACATGGACGTGCTCATCGTCCTGGCCA |
| | | CAAGCATTGCTTATGTTTATTCTCTGGTCATCCTGGTGGTTGCTGTG |
| | | GCTGAGAAGGCGGAGAGGAGCCCTGTGACATTCTTCGACACGCCC |
| | | CCCATGCTCTTTGTGTTCATTGCCCTGGGCCGGTGGCTGGAACACT |
| | | TGGCAAAGAGCAAAACCTCAGAAGCCCTGGCTAAACTCATGTCTC |
| | | TCCAAGCCACAGAAGCCACCGTTGTGACCCTTGGTGAGGACAATTT |
| | | AATCATCAGGGAGGAGCAAGTCCCCATGGAGCTGGTGCAGCGGGG |
| | | CGATATCGTCAAGGTGGTCCCTGGGGGAAAGTTTCCAGTGGATGG |
| | | GAAAGTCCTGGAAGGCAATACCATGGCTGATGAGTCCCTCATCAC |
| | | AGGAGAAGCCATGCCAGTCACTAAGAAACCCGGAAGCACTGTAAT |
| | | TGCGGGGTCTATAAATGCACATGGCTCTGTGCTCATTAAAGCTACC |
| | | CACGTGGGCAATGACACCACTTTGGCTCAGATTGTGAAACTGGTG |
| | | GAAGAGGCTCAGATGTCAAAGGCACCCATTCAGCAGCTGGCTGAC |
| | | CGGTTTAGTGGATATTTTGTCCCATTTATCATCATCATGTCAACTTT |
| | | GACGTTGGTGGTATGGATTGTAATCGGTTTTATCGATTTTGGTGTT |
| | | GTTCAGAGATACTTTCCTAACCCCAACAAGCACATCTCCCAGACAG |
| | | AGGTGATCATCCGGTTTGCTTTCCAGACGTCCATCACGGTGCTGTG |
| | | CATTGCCTGCCCCTGCTCCCTGGGGCTGGCCACGCCCACGGCTGTC |
| | | ATGGTGGGCACCGGGGTGGCCGCGCAGAACGGCATCCTCATCAAG |
| | | GGAGGCAAGCCCCTGGAGATGGCGCACAAGATAAAGACTGTGATG |
| | | TTTGACAAGACTGGCACCATTACCCATGGCGTCCCCAGGGTCATGC |
| | | GGGTGCTCCTGCTGGGGGATGTGGCCACACTGCCCCTCAGGAAGG |
| | | TTCTGGCTGTGGTGGGGACTGCGGAGGCCAGCAGTGAACACCCCT |
| | | TGGGCGTGGCAGTCACCAAATACTGTAAAGAGGAACTTGGAACAG |
| | | AGACCTTGGGATACTGCACGGACTTCCAGGCAGTGCCAGGCTGTG |
| | | GAATTGGGTGCAAAGTCAGCAACGTGGAAGGCATCCTGGCCCACA |
| | | GTGAGCGCCCTTTGAGTGCACCGGCCAGTCACCTGAATGAGGCTG |
| | | GCAGCCTTCCCGCAGAAAAAGATGCAGTCCCCCAGACCTTCTCTGT |
| | | GCTGATTGGAAACCGTGAGTGGCTGAGGCGCAACGGTTTAACCAT |
| | | TTCTAGCGATGTCAGTGACGCTATGACAGACCACGAGATGAAAGG |
| | | ACAGACAGCCATCCTGGTGGCTATTGACGGTGTGCTCTGTGGGATG |
| | | ATCGCAATCGCAGACGCTGTCAAGCAGGAGGCTGCCCTGGCTGTG |
| | | CACACGCTGCAGAGCATGGGTGTGGACGTGGTTCTGATCACGGGG |
| | | GACAACCGGAAGACAGCCAGAGCTATTGCCACCCAGGTTGGCATC |
| | | AACAAAGTCTTTGCAGAGGTGCTGCCTTCGCACAAGGTGGCCAAG |
| | | GTCCAGGAGCTCCAGAATAAAGGGAAGAAAGTCGCCATGGTGGGG |
| | | GATGGGGTCAATGACTCCCCGGCCTTGGCCCAGGCAGACATGGGT |
| | | GTGGCCATTGGCACCGGCACGGATGTGGCCATCGAGGCAGCCGAC |
| | | GTCGTCCTTATCAGAAATGATTTGCTGGATGTGGTGGCTAGCATTC |
| | | ACCTTTCCAAGAGGACTGTCCGAAGGATACGCATCAACCTGGTCCT |
| | | GGCACTGATTTATAACCTGGTTGGGATACCCATTGCAGCAGGTGTC |
| | | TTCATGCCCATCGGCATTGTGCTGCAGCCCTGGATGGGCTCAGCGG |
| | | CCATGGCAGCCTCCTCTGTGTCTGTGGTGCTCTCATCCCTGCAGCTC |
| | | AAGTGCTATAAGAAGCCTGACCTGGAGAGGTATGAGGCACAGGCG |
| | | CATGGCCACATGAAGCCCCTGACGGCATCCCAGGTCAGTGTGCAC |
| | | ATAGGCATGGATGACAGGTGGCGGGACTCCCCCAGGGCCACACCA |
| | | TGGGACCAGGTCAGCTATGTCAGCCAGGTGTCGCTGTCCTCCCTGA |
| | | CGTCCGACAAGCCATCTCGGCACAGCGCTGCAGCAGACGATGATG |
| | | GGGACAAGTGGTCTCTGCTCCTGAATGGCAGGGATGAGGAGCAGT |
| | | ACATCTGAAATAAAAGATCTTTATTTTCATTAGATCTGTGTGTTGG |
| | | TTTTTTGTGTGTCTAGAGTAGATAAGTAGCATGGCGGGTTAATCAT |
| | | TAACTACAAGGAACCCCTAGTGATGGAGTTGGCCACTCCCTCTCTG |
| | | CGCGCTCGCTCGCTCACTGAGGCCGGGCGACCAAAGGTCGCCCGA |
| | | CGCCCGGGCTTTGCCCGGGCGGCCTCAGTGAGCGAGCGAGCGCGC |
| | | AGAGAGGGAGTGGCCAA |
| 61 | A8 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAACGCGTCGCCTCGAGGTCAATTCACGCGA |
| | | GTTAATAATTACCAGCGCGGGCCAAATAAATAATCCGCGAGGGGC |
| | | AGGTGACGTTTGCCCAGCGCGCGCTGGTAATTATTAACCTCGCGAA |
| | | TATTGATTCGAGGCCGCGATTGCCGCAATCGCGAGGGGCAGGTGA |
| | | CCTTTGCCCAGCGCGCGTTCGCCCCGCCCCGGACGGTATCGATAAG |
| | | CTTAGGAGCTTGGGCTGCAGGTCGAGGGCACTGGGAGGATGTTGA |
| | | GTAAGATGGAAAACTACTGATGACCCTTGCAGAGACAGAGTATTA |
| | | GGACATGTTTGAACAGGGGCCGGGCGATCAGCAGGTAGCTCTAGA |
| | | GGATCCCCGTCTGTCTGCACATTTCGTAGAGCGAGTGTTCCGATAC |
| | | TCTAATCTCCCTAGGCAAGGTTCATATTTGTGTAGGTTACTTATTCT |
| | | CCTTTTGTTGACTAAGTCAATAATCAGAATCAGCAGGTTTGGAGTC |
| | | AGCTTGGCAGGGATCAGCAGCCTGGGTTGGAAGGAGGGGGTATAA |
| | | AAGCCCCTTCACCAGGAGAAGCCGTCACACAGATCCACAAGCTCC |
| | | TGCGGCCGCCACCATGCCTGAGCAGGAGAGACAGATCACAGCCAG |
| | | AGAAGGGGCCAGTCGGAAAATCTTATCTAAGCTTTCTTTGCCTACC |
| | | CGTGCCTGGGAACCAGCAATGAAGAAGAGTTTTGCTTTTGACAAT |
| | | GTTGGCTATGAAGGTGGTCTGGATGGCCTGGGCCCTTCTTCTCAGG |
| | | TGGCCACCAGCACAGTCAGGATCTTGGGCATGACTTGCCAGTCATG |
| | | TGTGAAGTCCATTGAGGACAGGATTTCCAATTTGAAAGGCATCATC |
| | | AGCATGAAGGTTTCCCTGGAACAAGGCAGTGCCACTGTGAAATAT |
| | | GTGCCATCGGTTGTGTGCCTGCAACAGGTTTGCCATCAAATTGGGG |
| | | ACATGGGCTTCGAGGCCAGCATTCCTGATGGAGCCGAAGGGAGTG |
| | | GGACAGATCACAGGTCTTCCAGTTCTCATTCCCCTGGCTCCCCACC |
| | | GAGAAACCAGGTCCAGGGCACATGCAGTAAGTGCTTCTTACAGAT |
| | | CAAAGGCATGACCTGTGCATCCTGTGTGTCTAACATAGAAAGGAA |
| | | TCTGCAGAAAGAAGCTGGTGTTCTCTCCGTGTTGGTTGCCTTGATG |
| | | GCAGGAAAGGCAGAGATCAAGTATGACCCAGAGGTCATCCAGCCC |
| | | CTCGAGATAGCTCAGTTCATCCAGGACCTGGGTTTTGAGGCAGCAG |
| | | TCATGGAGGACTACGCAGGCTCCGATGGCAACATTGAGCTGACAA |
| | | TCACAGGGATGACCTGCGCGTCCTGTGTCCACAACATAGAGTCCA |
| | | AACTCACGAGGACAAATGGCATCACTTATGCCTCCGTTGCCCTTGC |
| | | CACCAGCAAAGCCCTTGTTAAGTTTGACCCGGAAATTATCGGTCCA |
| | | CGGGATATTATCAAAATTATTGAGGAAATTGGCTTTCATGCTTCCC |
| | | TGGCCCAGAGAAACCCCAACGCTCATCACTTGGACCACAAGATGG |
| | | AAATAAAGCAGTGGAAGAAGTCTTTTCTTGTGAGTCTGTTTTTCTG |
| | | TATTCCTGTAATGGGGCTGATGATATATATGATGGTTATGGACCAC |
| | | CACTTTGCAACTCTTCACCATAATCAAAACATGAGTAAAGAAGAA |
| | | ATGATCAACCTTCATTCTTCTATGTTCCTGGAGCGCCAGATTCTTCC |
| | | AGGATTGTCTGTTATGAATTTGCTGTCCTTTTTATTGTGTGTACCTG |
| | | TACAGTTTTTCGGAGGCTGGTACTTCTACATTCAGGCTTATAAAGC |
| | | ACTGAAGCATAAGACAGCAAATATGGACGTACTGATTGTGCTGGC |
| | | AACCACCATTGCATTTGCCTACTCTTTGATTATTCTTCTAGTTGCAA |
| | | TGTATGAGAGAGCCAAAGTGAACCCTATTACTTTCTTTGACACACC |
| | | CCCTATGCTGTTTGTGTTTATTGCACTAGGCCGATGGCTGGAACAT |
| | | ATAGCAAAGGGCAAAACATCAGAGGCTCTTGCAAAGTTAATTTCA |
| | | CTACAAGCTACAGAAGCAACTATTGTAACTCTTGATTCTGATAATA |
| | | TCCTCCTCAGTGAAGAACAAGTGGATGTGGAACTTGTACAACGTG |
| | | GAGATATCATTAAAGTAGTTCCAGGAGGCAAATTTCCAGTGGATG |
| | | GTCGTGTTATTGAAGGACATTCTATGGTAGATGAGTCCCTCATCAC |
| | | AGGGGAGGCAATGCCTGTGGCTAAGAAACCTGGCAGCACAGTGAT |
| | | TGCTGGTTCCATTAACCAGAACGGGTCACTGCTTATCTGCGCAACA |
| | | CATGTTGGAGCAGACACAACCCTTTCTCAAATTGTCAAACTTGTGG |
| | | AAGAGGCACAAACATCAAAGGCTCCTATCCAGCAGTTTGCAGACA |
| | | AACTCAGTGGCTATTTTGTTCCTTTTATTGTTTTTGTTTCCATTGCCA |
| | | CCCTCTTGGTATGGATTGTAATTGGATTTCTGAATTTTGAAATTGTG |
| | | GAAACCTACTTTCCTGGCTACAATAGAAGTATCTCCCGAACAGAA |
| | | ACGATAATACGATTTGCTTTCCAAGCCTCTATCACAGTTCTGTGTA |
| | | TTGCATGTCCCTGTTCACTGGGACTGGCCACTCCAACTGCTGTGAT |
| | | GGTGGGTACAGGAGTAGGTGCTCAAAATGGCATACTAATAAAAGG |
| | | TGGAGAGCCATTGGAGATGGCTCATAAGGTAAAGGTAGTGGTATT |
| | | TGATAAGACTGGAACCATTACTCACGGAACCCCAGTGGTGAATCA |
| | | AGTAAAGGTTCTAACTGAAAGTAACAGAATATCACACCATAAAAT |
| | | CTTGGCCATTGTGGGAACTGCTGAAAGTAACAGTGAACACCCTCTA |
| | | GGAACAGCCATAACCAAATATTGCAAACAGGAGCTGGACACTGAA |
| | | ACCTTGGGTACCTGCATAGATTTCCAGGTTGTGCCAGGCTGTGGTA |
| | | TTAGCTGTAAAGTCACCAATATTGAAGGCTTGCTACATAAGAATAA |
| | | CTGGAATATAGAGGACAATAATATTAAAAATGCATCCCTGGTTCA |
| | | AATTGATGCCAGTAATGAACAGTCATCAACTTCGTCTTCCATGATT |
| | | ATTGATGCCCAGATCTCAAATGCTCTTAATGCTCAGCAGTATAAAG |
| | | TCCTCATTGGTAACCGGGAGTGGATGATTAGAAATGGTCTTGTCAT |
| | | TAATAACGATGTAAATGATTTCATGACTGAACATGAGAGAAAAGG |
| | | TCGGACTGCTGTATTAGTAGCAGTTGATGATGAGCTGTGTGGCTTG |
| | | ATAGCCATTGCAGACACAGTGAAGCCTGAAGCAGAACTGGCTATC |
| | | CATATTCTGAAATCTATGGGCTTAGAAGTAGTTCTGATGACTGGAG |
| | | ACAACAGTAAAACAGCTAGATCTATTGCTTCTCAGGTTGGCATTAC |
| | | TAAGGTGTTTGCTGAAGTTCTACCTTCTCACAAGGTTGCTAAAGTG |
| | | AAGCAACTTCAAGAGGAGGGGAAACGGGTAGCAATGGTGGGAGA |
| | | TGGAATCAATGACTCCCCAGCTCTGGCAATGGCTAATGTGGGAATT |
| | | GCTATTGGCACAGGCACAGATGTAGCCATTGAAGCAGCTGATGTG |
| | | GTTTTGATAAGGAATGATCTTCTGGATGTAGTGGCAAGTATTGACT |
| | | TATCAAGAAAGACAGTCAAGAGGATTCGGATAAATTTTGTCTTTGC |
| | | TCTAATTTATAATCTGGTTGGAATTCCCATAGCTGCTGGAGTTTTTA |
| | | TGCCCATTGGTTTGGTTTTGCAGCCCTGGATGGGATCTGCAGCAAT |
| | | GGCTGCTTCATCTGTTTCTGTAGTACTTTCTTCTCTCCAGCTCAAGT |
| | | GCTATAAGAAGCCTGACCTGGAGAGGTATGAGGCACAGGCGCATG |
| | | GCCACATGAAGCCCCTGACGGCATCCCAGGTCAGTGTGCACATAG |
| | | GCATGGATGACAGGTGGCGGGACTCCCCCAGGGCCACACCATGGG |
| | | ACCAGGTCAGCTATGTCAGCCAGGTGTCGCTGTCCTCCCTGACGTC |
| | | CGACAAGCCATCTCGGCACAGCGCTGCAGCAGACGATGATGGGGA |
| | | CAAGTGGTCTCTGCTCCTGAATGGCAGGGATGAGGAGCAGTACAT |
| | | CTGAAATAAAAGATCTTTATTTTCATTAGATCTGTGTGTTGGTTTTT |
| | | TGTGTGTCTAGAGTAGATAAGTAGCATGGCGGGTTAATCATTAACT |
| | | ACAAGGAACCCCTAGTGATGGAGTTGGCCACTCCCTCTCTGCGCGC |
| | | TCGCTCGCTCACTGAGGCCGGGCGACCAAAGGTCGCCCGACGCCC |
| | | GGGCTTTGCCCGGGCGGCCTCAGTGAGCGAGCGAGCGCGCAGAGA |
| | | GGGAGTGGCCAA |
| 62 | A9 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAACGCGTCGCCTCGAGGTCAATTCACGCGA |
| | | GTTAATAATTACCAGCGCGGGCCAAATAAATAATCCGCGAGGGGC |
| | | AGGTGACGTTTGCCCAGCGCGCGCTGGTAATTATTAACCTCGCGAA |
| | | TATTGATTCGAGGCCGCGATTGCCGCAATCGCGAGGGGCAGGTGA |
| | | CCTTTGCCCAGCGCGCGTTCGCCCCGCCCCGGACGGTATCGATAAG |
| | | CTTAGGAGCTTGGGCTGCAGGTCGAGGGCACTGGGAGGATGTTGA |
| | | GTAAGATGGAAAACTACTGATGACCCTTGCAGAGACAGAGTATTA |
| | | GGACATGTTTGAACAGGGGCCGGGCGATCAGCAGGTAGCTCTAGA |
| | | GGATCCCCGTCTGTCTGCACATTTCGTAGAGCGAGTGTTCCGATAC |
| | | TCTAATCTCCCTAGGCAAGGTTCATATTTGTGTAGGTTACTTATTCT |
| | | CCTTTTGTTGACTAAGTCAATAATCAGAATCAGCAGGTTTGGAGTC |
| | | AGCTTGGCAGGGATCAGCAGCCTGGGTTGGAAGGAGGGGGTATAA |
| | | AAGCCCCTTCACCAGGAGAAGCCGTCACACAGATCCACAAGCTCC |
| | | TGCGGCCGCCACCATGCCTGAGCAGGAGAGACAGATCACAGCCAG |
| | | AGAAGGGGCCAGTCGGAAAATCTTATCTAAGCTTTCTTTGCCTACC |
| | | CGTGCCTGGGAACCAGCAATGAAGAAGAGTTTTGCTTTTGACAAT |
| | | GTTGGCTATGAAGGTGGTCTGGATGGCCTGGGCCCTTCTTCTCAGG |
| | | TGGCCCCTGATGGAGCCGAAGGGAGTGGGACAGATCACAGGTCTT |
| | | CCAGTTCTCATTCCCCTGGCTCCCCACCGAGAAACCAGGTCCAGGG |
| | | CACATGCAGTACCACTCTGATTGCCATTGCCGGCATGACCTGTGCA |
| | | TCCTGTGTCCATTCCATTGAAGGCATGATCTCCCAACTGGAAGGGG |
| | | TGCAGCAAATATCGGTGTCTTTGGCCGAAGGGACTGCAACAGTTCT |
| | | TTATAATCCCTCTGTAATTAGCCCAGAAGAACTCAGAGCTGCTATA |
| | | GAAGACATGGGATTTGAGGCTTCAGTCGTTTCTGAAAGCTGTTCTA |
| | | CTAACCCTCTTGGAAACCACAGTGCTGGGAATTCCATGGTGCAAAC |
| | | TACAGATGGTACACCTACATCTGTGCAGGAAGTGGCTCCCCACACT |
| | | GGGAGGCTCCCTGCAAACCATGCCCCGGACATCTTGGCAAAGTCC |
| | | CCACAATCAACCAGAGCAGTGGCACCGCAGAAGTGCTTCTTACAG |
| | | ATCAAAGGCATGACCTGTGCATCCTGTGTGTCTAACATAGAAAGG |
| | | AATCTGCAGAAAGAAGCTGGTGTTCTCTCCGTGTTGGTTGCCTTGA |
| | | TGGCAGGAAAGGCAGAGATCAAGTATGACCCAGAGGTCATCCAGC |
| | | CCCTCGAGATAGCTCAGTTCATCCAGGACCTGGGTTTTGAGGCAGC |
| | | AGTCATGGAGGACTACGCAGGCTCCGATGGCAACATTGAGCTGAC |
| | | AATCACAGGGATGACCTGCGCGTCCTGTGTCCACAACATAGAGTC |
| | | CAAACTCACGAGGACAAATGGCATCACTTATGCCTCCGTTGCCCTT |
| | | GCCACCAGCAAAGCCCTTGTTAAGTTTGACCCGGAAATTATCGGTC |
| | | CACGGGATATTATCAAAATTATTGAGGAAATTGGCTTTCATGCTTC |
| | | CCTGGCCCAGAGAAACCCCAACGCTCATCACTTGGACCACAAGAT |
| | | GGAAATAAAGCAGTGGAAGAAGTCTTTTCTTGTGAGTCTGTTTTTC |
| | | TGTATTCCTGTAATGGGGCTGATGATATATATGATGGTTATGGACC |
| | | ACCACTTTGCAACTCTTCACCATAATCAAAACATGAGTAAAGAAG |
| | | AAATGATCAACCTTCATTCTTCTATGTTCCTGGAGCGCCAGATTCTT |
| | | CCAGGATTGTCTGTTATGAATTTGCTGTCCTTTTTATTGTGTGTACC |
| | | TGTACAGTTTTTCGGAGGCTGGTACTTCTACATTCAGGCTTATAAA |
| | | GCACTGAAGCATAAGACAGCAAATATGGACGTACTGATTGTGCTG |
| | | GCAACCACCATTGCATTTGCCTACTCTTTGATTATTCTTCTAGTTGC |
| | | AATGTATGAGAGAGCCAAAGTGAACCCTATTACTTTCTTTGACACA |
| | | CCCCCTATGCTGTTTGTGTTTATTGCACTAGGCCGATGGCTGGAAC |
| | | ATATAGCAAAGGGCAAAACATCAGAGGCTCTTGCAAAGTTAATTT |
| | | CACTACAAGCTACAGAAGCAACTATTGTAACTCTTGATTCTGATAA |
| | | TATCCTCCTCAGTGAAGAACAAGTGGATGTGGAACTTGTACAACGT |
| | | GGAGATATCATTAAAGTAGTTCCAGGAGGCAAATTTCCAGTGGAT |
| | | GGTCGTGTTATTGAAGGACATTCTATGGTAGATGAGTCCCTCATCA |
| | | CAGGGGAGGCAATGCCTGTGGCTAAGAAACCTGGCAGCACAGTGA |
| | | TTGCTGGTTCCATTAACCAGAACGGGTCACTGCTTATCTGCGCAAC |
| | | ACATGTTGGAGCAGACACAACCCTTTCTCAAATTGTCAAACTTGTG |
| | | GAAGAGGCACAAACATCAAAGGCTCCTATCCAGCAGTTTGCAGAC |
| | | AAACTCAGTGGCTATTTTGTTCCTTTTATTGTTTTTGTTTCCATTGCC |
| | | ACCCTCTTGGTATGGATTGTAATTGGATTTCTGAATTTTGAAATTGT |
| | | GGAAACCTACTTTCCTGGCTACAATAGAAGTATCTCCCGAACAGA |
| | | AACGATAATACGATTTGCTTTCCAAGCCTCTATCACAGTTCTGTGT |
| | | ATTGCATGTCCCTGTTCACTGGGACTGGCCACTCCAACTGCTGTGA |
| | | TGGTGGGTACAGGAGTAGGTGCTCAAAATGGCATACTAATAAAAG |
| | | GTGGAGAGCCATTGGAGATGGCTCATAAGGTAAAGGTAGTGGTAT |
| | | TTGATAAGACTGGAACCATTACTCACGGAACCCCAGTGGTGAATC |
| | | AAGTAAAGGTTCTAACTGAAAGTAACAGAATATCACACCATAAAA |
| | | TCTTGGCCATTGTGGGAACTGCTGAAAGTAACAGTGAACACCCTCT |
| | | AGGAACAGCCATAACCAAATATTGCAAACAGGAGCTGGACACTGA |
| | | AACCTTGGGTACCTGCATAGATTTCCAGGTTGTGCCAGGCTGTGGT |
| | | ATTAGCTGTAAAGTCACCAATATTGAAGGCTTGCTACATAAGAATA |
| | | ACTGGAATATAGAGGACAATAATATTAAAAATGCATCCCTGGTTC |
| | | AAATTGATGCCAGTAATGAACAGTCATCAACTTCGTCTTCCATGAT |
| | | TATTGATGCCCAGATCTCAAATGCTCTTAATGCTCAGCAGTATAAA |
| | | GTCCTCATTGGTAACCGGGAGTGGATGATTAGAAATGGTCTTGTCA |
| | | TTAATAACGATGTAAATGATTTCATGACTGAACATGAGAGAAAAG |
| | | GTCGGACTGCTGTATTAGTAGCAGTTGATGATGAGCTGTGTGGCTT |
| | | GATAGCCATTGCAGACACAGTGAAGCCTGAAGCAGAACTGGCTAT |
| | | CCATATTCTGAAATCTATGGGCTTAGAAGTAGTTCTGATGACTGGA |
| | | GACAACAGTAAAACAGCTAGATCTATTGCTTCTCAGGTTGGCATTA |
| | | CTAAGGTGTTTGCTGAAGTTCTACCTTCTCACAAGGTTGCTAAAGT |
| | | GAAGCAACTTCAAGAGGAGGGGAAACGGGTAGCAATGGTGGGAG |
| | | ATGGAATCAATGACTCCCCAGCTCTGGCAATGGCTAATGTGGGAA |
| | | TTGCTATTGGCACAGGCACAGATGTAGCCATTGAAGCAGCTGATGT |
| | | GGTTTTGATAAGGAATGATCTTCTGGATGTAGTGGCAAGTATTGAC |
| | | TTATCAAGAAAGACAGTCAAGAGGATTCGGATAAATTTTGTCTTTG |
| | | CTCTAATTTATAATCTGGTTGGAATTCCCATAGCTGCTGGAGTTTTT |
| | | ATGCCCATTGGTTTGGTTTTGCAGCCCTGGATGGGATCTGCAGCAA |
| | | TGGCTGCTTCATCTGTTTCTGTAGTACTTTCTTCTCTCCAGCTCAAG |
| | | TGCTATAAGAAGCCTGACCTGGAGAGGTATGAGGCACAGGCGCAT |
| | | GGCCACATGAAGCCCCTGACGGCATCCCAGGTCAGTGTGCACATA |
| | | GGCATGGATGACAGGTGGCGGGACTCCCCCAGGGCCACACCATGG |
| | | GACCAGGTCAGCTATGTCAGCCAGGTGTCGCTGTCCTCCCTGACGT |
| | | CCGACAAGCCATCTCGGCACAGCGCTGCAGCAGACGATGATGGGG |
| | | ACAAGTGGTCTCTGCTCCTGAATGGCAGGGATGAGGAGCAGTACA |
| | | TCTGAAATAAAAGATCTTTATTTTCATTAGATCTGTGTGTTGGTTTT |
| | | TTGTGTGTCTAGAGTAGATAAGTAGCATGGCGGGTTAATCATTAAC |
| | | TACAAGGAACCCCTAGTGATGGAGTTGGCCACTCCCTCTCTGCGCG |
| | | CTCGCTCGCTCACTGAGGCCGGGCGACCAAAGGTCGCCCGACGCC |
| | | CGGGCTTTGCCCGGGCGGCCTCAGTGAGCGAGCGAGCGCGCAGAG |
| | | AGGGAGTGGCCAA |
| 63 | A10 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAACGCGTCGCCTCGAGGTCAATTCACGCGA |
| | | GTTAATAATTACCAGCGCGGGCCAAATAAATAATCCGCGAGGGGC |
| | | AGGTGACGTTTGCCCAGCGCGCGCTGGTAATTATTAACCTCGCGAA |
| | | TATTGATTCGAGGCCGCGATTGCCGCAATCGCGAGGGGCAGGTGA |
| | | CCTTTGCCCAGCGCGCGTTCGCCCCGCCCCGGACGGTATCGATAAG |
| | | CTTAGGAGCTTGGGCTGCAGGTCGAGGGCACTGGGAGGATGTTGA |
| | | GTAAGATGGAAAACTACTGATGACCCTTGCAGAGACAGAGTATTA |
| | | GGACATGTTTGAACAGGGGCCGGGCGATCAGCAGGTAGCTCTAGA |
| | | GGATCCCCGTCTGTCTGCACATTTCGTAGAGCGAGTGTTCCGATAC |
| | | TCTAATCTCCCTAGGCAAGGTTCATATTTGTGTAGGTTACTTATTCT |
| | | CCTTTTGTTGACTAAGTCAATAATCAGAATCAGCAGGTTTGGAGTC |
| | | AGCTTGGCAGGGATCAGCAGCCTGGGTTGGAAGGAGGGGGTATAA |
| | | AAGCCCCTTCACCAGGAGAAGCCGTCACACAGATCCACAAGCTCC |
| | | TGCGGCCGCCACCATGCCTGAGCAGGAGAGACAGATCACAGCCAG |
| | | AGAAGGGGCCAGTCGGAAAATCTTATCTAAGCTTTCTTTGCCTACC |
| | | CGTGCCTGGGAACCAGCAATGAAGAAGAGTTTTGCTTTTGACAAT |
| | | GTTGGCTATGAAGGTGGTCTGGATGGCCTGGGCCCTTCTTCTCAGG |
| | | TGGCCCCTGATGGAGCCGAAGGGAGTGGGACAGATCACAGGTCTT |
| | | CCAGTTCTCATTCCCCTGGCTCCCCACCGAGAAACCAGGTCCAGGG |
| | | CACATGCAGTAAGTGCTTCTTACAGATCAAAGGCATGACCTGTGCA |
| | | TCCTGTGTGTCTAACATAGAAAGGAATCTGCAGAAAGAAGCTGGT |
| | | GTTCTCTCCGTGTTGGTTGCCTTGATGGCAGGAAAGGCAGAGATCA |
| | | AGTATGACCCAGAGGTCATCCAGCCCCTCGAGATAGCTCAGTTCAT |
| | | CCAGGACCTGGGTTTTGAGGCAGCAGTCATGGAGGACTACGCAGG |
| | | CTCCGATGGCAACATTGAGCTGACAATCACAGGGATGACCTGCGC |
| | | GTCCTGTGTCCACAACATAGAGTCCAAACTCACGAGGACAAATGG |
| | | CATCACTTATGCCTCCGTTGCCCTTGCCACCAGCAAAGCCCTTGTT |
| | | AAGTTTGACCCGGAAATTATCGGTCCACGGGATATTATCAAAATTA |
| | | TTGAGGAAATTGGCTTTCATGCTTCCCTGGCCCAGAGAAACCCCAA |
| | | CGCTCATCACTTGGACCACAAGATGGAAATAAAGCAGTGGAAGAA |
| | | GTCTTTTCTTGTGAGTCTGTTTTTCTGTATTCCTGTAATGGGGCTGA |
| | | TGATATATATGATGGTTATGGACCACCACTTTGCAACTCTTCACCA |
| | | TAATCAAAACATGAGTAAAGAAGAAATGATCAACCTTCATTCTTCT |
| | | ATGTTCCTGGAGCGCCAGATTCTTCCAGGATTGTCTGTTATGAATT |
| | | TGCTGTCCTTTTTATTGTGTGTACCTGTACAGTTTTTCGGAGGCTGG |
| | | TACTTCTACATTCAGGCTTATAAAGCACTGAAGCATAAGACAGCA |
| | | AATATGGACGTACTGATTGTGCTGGCAACCACCATTGCATTTGCCT |
| | | ACTCTTTGATTATTCTTCTAGTTGCAATGTATGAGAGAGCCAAAGT |
| | | GAACCCTATTACTTTCTTTGACACACCCCCTATGCTGTTTGTGTTTA |
| | | TTGCACTAGGCCGATGGCTGGAACATATAGCAAAGGGCAAAACAT |
| | | CAGAGGCTCTTGCAAAGTTAATTTCACTACAAGCTACAGAAGCAA |
| | | CTATTGTAACTCTTGATTCTGATAATATCCTCCTCAGTGAAGAACA |
| | | AGTGGATGTGGAACTTGTACAACGTGGAGATATCATTAAAGTAGT |
| | | TCCAGGAGGCAAATTTCCAGTGGATGGTCGTGTTATTGAAGGACAT |
| | | TCTATGGTAGATGAGTCCCTCATCACAGGGGAGGCAATGCCTGTG |
| | | GCTAAGAAACCTGGCAGCACAGTGATTGCTGGTTCCATTAACCAG |
| | | AACGGGTCACTGCTTATCTGCGCAACACATGTTGGAGCAGACACA |
| | | ACCCTTTCTCAAATTGTCAAACTTGTGGAAGAGGCACAAACATCAA |
| | | AGGCTCCTATCCAGCAGTTTGCAGACAAACTCAGTGGCTATTTTGT |
| | | TCCTTTTATTGTTTTTGTTTCCATTGCCACCCTCTTGGTATGGATTGT |
| | | AATTGGATTTCTGAATTTTGAAATTGTGGAAACCTACTTTCCTGGC |
| | | TACAATAGAAGTATCTCCCGAACAGAAACGATAATACGATTTGCTT |
| | | TCCAAGCCTCTATCACAGTTCTGTGTATTGCATGTCCCTGTTCACTG |
| | | GGACTGGCCACTCCAACTGCTGTGATGGTGGGTACAGGAGTAGGT |
| | | GCTCAAAATGGCATACTAATAAAAGGTGGAGAGCCATTGGAGATG |
| | | GCTCATAAGGTAAAGGTAGTGGTATTTGATAAGACTGGAACCATT |
| | | ACTCACGGAACCCCAGTGGTGAATCAAGTAAAGGTTCTAACTGAA |
| | | AGTAACAGAATATCACACCATAAAATCTTGGCCATTGTGGGAACT |
| | | GCTGAAAGTAACAGTGAACACCCTCTAGGAACAGCCATAACCAAA |
| | | TATTGCAAACAGGAGCTGGACACTGAAACCTTGGGTACCTGCATA |
| | | GATTTCCAGGTTGTGCCAGGCTGTGGTATTAGCTGTAAAGTCACCA |
| | | ATATTGAAGGCTTGCTACATAAGAATAACTGGAATATAGAGGACA |
| | | ATAATATTAAAAATGCATCCCTGGTTCAAATTGATGCCAGTAATGA |
| | | ACAGTCATCAACTTCGTCTTCCATGATTATTGATGCCCAGATCTCA |
| | | AATGCTCTTAATGCTCAGCAGTATAAAGTCCTCATTGGTAACCGGG |
| | | AGTGGATGATTAGAAATGGTCTTGTCATTAATAACGATGTAAATGA |
| | | TTTCATGACTGAACATGAGAGAAAAGGTCGGACTGCTGTATTAGT |
| | | AGCAGTTGATGATGAGCTGTGTGGCTTGATAGCCATTGCAGACAC |
| | | AGTGAAGCCTGAAGCAGAACTGGCTATCCATATTCTGAAATCTATG |
| | | GGCTTAGAAGTAGTTCTGATGACTGGAGACAACAGTAAAACAGCT |
| | | AGATCTATTGCTTCTCAGGTTGGCATTACTAAGGTGTTTGCTGAAG |
| | | TTCTACCTTCTCACAAGGTTGCTAAAGTGAAGCAACTTCAAGAGGA |
| | | GGGGAAACGGGTAGCAATGGTGGGAGATGGAATCAATGACTCCCC |
| | | AGCTCTGGCAATGGCTAATGTGGGAATTGCTATTGGCACAGGCAC |
| | | AGATGTAGCCATTGAAGCAGCTGATGTGGTTTTGATAAGGAATGA |
| | | TCTTCTGGATGTAGTGGCAAGTATTGACTTATCAAGAAAGACAGTC |
| | | AAGAGGATTCGGATAAATTTTGTCTTTGCTCTAATTTATAATCTGG |
| | | TTGGAATTCCCATAGCTGCTGGAGTTTTTATGCCCATTGGTTTGGTT |
| | | TTGCAGCCCTGGATGGGATCTGCAGCAATGGCTGCTTCATCTGTTT |
| | | CTGTAGTACTTTCTTCTCTCCAGCTCAAGTGCTATAAGAAGCCTGA |
| | | CCTGGAGAGGTATGAGGCACAGGCGCATGGCCACATGAAGCCCCT |
| | | GACGGCATCCCAGGTCAGTGTGCACATAGGCATGGATGACAGGTG |
| | | GCGGGACTCCCCCAGGGCCACACCATGGGACCAGGTCAGCTATGT |
| | | CAGCCAGGTGTCGCTGTCCTCCCTGACGTCCGACAAGCCATCTCGG |
| | | CACAGCGCTGCAGCAGACGATGATGGGGACAAGTGGTCTCTGCTC |
| | | CTGAATGGCAGGGATGAGGAGCAGTACATCTGAAATAAAAGATCT |
| | | TTATTTTCATTAGATCTGTGTGTTGGTTTTTTGTGTGTCTAGAGTAG |
| | | ATAAGTAGCATGGCGGGTTAATCATTAACTACAAGGAACCCCTAG |
| | | TGATGGAGTTGGCCACTCCCTCTCTGCGCGCTCGCTCGCTCACTGA |
| | | GGCCGGGCGACCAAAGGTCGCCCGACGCCCGGGCTTTGCCCGGGC |
| | | GGCCTCAGTGAGCGAGCGAGCGCGCAGAGAGGGAGTGGCCAA |
| 64 | A11 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTAATAATTACCAGCGCGGGCCAAAT |
| | | AAATAATCCGCGAGGGGCAGCTGGACTTTGGACTCCGCGCGCTGG |
| | | TAATTATTAACCTCGCGAATATTGATTCGAGGCCGCGATTGCCGCA |
| | | ATCGCGAGGGGCAGGTGACCTTTGCCCAGCGCGCGTTCGCCCCGC |
| | | CCCGGACGGTATCGATAAGCTTAGGAGCTTGGGCTGCAGGTCGAG |
| | | GGCACTGGGAGGATGTTGAGTAAGATGGAAAACTACTGATGACCC |
| | | TTGCAGAGACAGAGTATTAGGACATGTTTGAACAGGGGCCGGGCG |
| | | ATCAGCAGGTAGCTCTAGAGGATCCCCGTCTGTCTGCACATTTCGT |
| | | AGAGCGAGTGTTCCGATACTCTAATCTCCCTAGGCAAGGTTCATAT |
| | | TTGTGTAGGTTACTTATTCTCCTTTTGTTGACTAAGTCAATAATCAG |
| | | AATCAGCAGGTTTGGAGTCAGCTTGGCAGGGATCAGCAGCCTGGG |
| | | TTGGAAGGAGGGGGTATAAAAGCCCCTTCACCAGGAGAAGCCGTC |
| | | ACACAGATCCACAAGCTCGAGCCGAGTGGTCGTGCCTCCATAGAA |
| | | GCGGCCGCCACCATGCCGGAACAAGAACGCCAGATCACAGCAAGA |
| | | GAAGGAGCAAGTAGGAAGATCCTGAGCAAGCTCTCCCTGCCCACA |
| | | AGGGCCTGGGAGCCGGCCATGAAGAAGTCTTTTGCCTTCGATAAT |
| | | GTCGGTTATGAAGGCGGGCTCGATGGCCTGGGCCCCAGCAGCCAG |
| | | CCCCAGAAGTGTTTCCTGCAGATCAAGGGCATGACCTGTGCCAGCT |
| | | GTGTTAGCAATATCGAAAGAAACCTCCAAAAAGAGGCTGGAGTTC |
| | | TGAGTGTTCTAGTGGCTTTAATGGCCGGGAAAGCCGAGATCAAGT |
| | | ATGATCCCGAGGTGATTCAGCCATTGGAGATTGCCCAGTTCATCCA |
| | | GGATCTGGGCTTCGAGGCCGCCGTGATGGAGGATTACGCCGGCTC |
| | | TGATGGCAACATCGAACTGACCATCACCGGCATGACCTGTGCTTCC |
| | | TGCGTGCATAATATCGAGAGCAAACTGACGCGGACCAATGGAATC |
| | | ACCTATGCCAGCGTCGCCCTGGCAACCTCTAAGGCTCTGGTGAAGT |
| | | TTGACCCCGAAATCATTGGACCTAGAGACATCATCAAAATTATTGA |
| | | AGAGATTGGTTTCCACGCCAGCTTAGCTCAGCGGAATCCCAACGC |
| | | ACACCATCTTGACCATAAGATGGAAATCAAACAGTGGAAGAAGAG |
| | | CTTCTTGTGTAGCCTGGTCTTCGGGATACCCGTCATGGCACTGATG |
| | | ATTTACATGCTCATTCCGTCCAACGAGCCCCACCAGTCCATGGTGC |
| | | TGGACCACAACATCATCCCTGGGCTGTCTATCCTCAACCTGATCTT |
| | | CTTTATTCTGTGCACATTTGTCCAGCTCTTGGGAGGCTGGTATTTCT |
| | | ATGTACAGGCCTACAAATCCCTGAGGCATCGAAGCGCCAACATGG |
| | | ACGTTTTAATTGTGCTGGCTACATCCATTGCCTACGTGTACTCCCTG |
| | | GTGATCTTAGTGGTTGCTGTGGCTGAAAAGGCTGAGAGAAGCCCT |
| | | GTCACCTTCTTCGACACCCCCCCCATGCTCTTCGTGTTTATTGCACT |
| | | GGGTCGCTGGCTGGAGCATTTAGCCAAGTCCAAGACATCCGAGGC |
| | | TCTGGCCAAGCTGATGTCTCTACAGGCCACGGAGGCCACCGTGGT |
| | | GACTCTGGGTGAAGACAATTTGATAATACGCGAGGAACAGGTGCC |
| | | TATGGAGCTTGTGCAGCGCGGAGATATCGTCAAGGTGGTGCCCGG |
| | | CGGCAAGTTCCCAGTGGATGGAAAAGTGTTAGAGGGCAATACCAT |
| | | GGCCGATGAATCCCTCATCACTGGCGAGGCAATGCCGGTGACCAA |
| | | GAAACCTGGAAGTACTGTCATTGCAGGCAGCATCAATGCCCATGG |
| | | GAGCGTCCTGATCAAGGCCACCCACGTGGGCAATGATACAACTCT |
| | | CGCCCAGATCGTCAAGCTAGTGGAAGAGGCTCAGATGTCTAAAGC |
| | | TCCAATCCAGCAACTGGCTGACCGCTTCAGCGGCTACTTTGTCCCC |
| | | TTTATCATCATCATGAGCACCCTCACCCTGGTGGTGTGGATAGTCA |
| | | TAGGCTTCATTGACTTTGGCGTGGTGCAGAGATACTTCCCCAACCC |
| | | TAACAAACATATCTCCCAAACCGAGGTCATTATCAGATTCGCCTTT |
| | | CAGACATCTATCACTGTGCTGTGCATAGCCTGCCCTTGCTCTCTGG |
| | | GCCTGGCCACCCCCACCGCCGTGATGGTTGGAACCGGGGTCGCTG |
| | | CACAAAATGGGATCCTGATAAAAGGGGGCAAACCCCTGGAAATGG |
| | | CACATAAGATTAAAACAGTTATGTTTGATAAGACAGGCACCATTA |
| | | CTCACGGTGTCCCTCGAGTCATGAGAGTGCTGCTGCTGGGGGACGT |
| | | GGCTACACTGCCACTGCGCAAGGTGCTGGCTGTGGTGGGCACAGC |
| | | GGAGGCCTCCTCAGAACACCCATTAGGCGTAGCTGTCACAAAATA |
| | | CTGTAAGGAGGAGCTGGGGACAGAGACCCTCGGCTATTGTACAGA |
| | | CTTCCAGGCCGTGCCTGGCTGCGGCATCGGCTGTAAGGTCAGCAAT |
| | | GTGGAGGGCATCCTGGCACACAGCGAGAGACCCCTCTCTGCCCCA |
| | | GCCTCCCACCTGAATGAAGCTGGGTCTCTGCCTGCTGAGAAGGAC |
| | | GCCGTCCCTCAGACCTTCTCCGTGTTGATTGGGAACAGAGAGTGGC |
| | | TTAGAAGGAATGGCCTGACCATCAGCTCTGACGTCTCGGATGCCAT |
| | | GACAGATCACGAGATGAAAGGCCAGACAGCCATACTGGTTGCCAT |
| | | CGACGGCGTGCTCTGTGGCATGATTGCCATCGCTGATGCTGTTAAA |
| | | CAGGAAGCTGCGCTGGCAGTCCACACCCTCCAGTCCATGGGAGTG |
| | | GACGTGGTCCTGATTACTGGAGACAACAGGAAAACCGCTCGGGCC |
| | | ATCGCTACCCAAGTGGGGATAAATAAGGTTTTTGCAGAAGTCTTAC |
| | | CCTCCCACAAAGTAGCCAAAGTGCAGGAGCTCCAGAACAAGGGTA |
| | | AAAAGGTGGCAATGGTGGGAGATGGTGTGAATGACAGCCCAGCTC |
| | | TCGCGCAGGCTGACATGGGCGTAGCCATAGGTACTGGAACAGATG |
| | | TCGCGATTGAAGCCGCTGACGTAGTCCTCATCCGAAACGATTTGCT |
| | | GGATGTGGTGGCCAGCATCCATCTGTCCAAGCGTACCGTGCGCCG |
| | | GATCCGCATCAACCTGGTCTTGGCCCTCATCTATAATCTCGTGGGC |
| | | ATCCCCATCGCTGCTGGGGTCTTCATGCCCATCGGCATTGTGCTGC |
| | | AGCCCTGGATGGGAAGCGCTGCCATGGCTGCTTCTTCTGTCTCCGT |
| | | TGTGCTGTCCAGCCTACAGCTGAAGTGCTATAAAAAGCCAGACCTC |
| | | GAGCGCTACGAAGCCCAGGCGCATGGCCATATGAAGCCCCTCACG |
| | | GCTTCCCAGGTCTCGGTTCACATTGGGATGGATGACAGATGGCGG |
| | | GACTCCCCACGCGCCACACCCTGGGACCAGGTATCCTATGTGAGCC |
| | | AGGTGAGCCTCAGCTCCCTGACATCAGATAAACCGTCTAGGCACTC |
| | | AGCTGCCGCAGATGATGACGGTGACAAATGGAGCCTCCTCCTGAA |
| | | CGGCCGCGACGAGGAGCAGTACATCTAAAATAAAAGATCTTTATT |
| | | TTCATTAGATCTGTGTGTTGGTTTTTTGTGTGTCTAGAGTAGATAAG |
| | | TAGCATGGCGGGTTAATCATTAACTACAAGGAACCCCTAGTGATG |
| | | GAGTTGGCCACTCCCTCTCTGCGCGCTCGCTCGCTCACTGAGGCCG |
| | | GGCGACCAAAGGTCGCCCGACGCCCGGGCTTTGCCCGGGCGGCCT |
| | | CAGTGAGCGAGCGAGCGCGCAGAGAGGGAGTGGCCAA |
| 65 | A12 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTAATAATTACCAGCGCGGGCCAAAT |
| | | AAATAATCCGCGAGGGGCAGCTGGACTTTGGACTCCGCGCGCTGG |
| | | TAATTATTAACCTCGCGAATATTGATTCGAGGCCGCGATTGCCGCA |
| | | ATCGCGAGGGGCAGGTGACCTTTGCCCAGCGCGCGTTCGCCCCGC |
| | | CCCGGACGGTATCGATAAGCTTAGGAGCTTGGGCTGCAGGTCGAG |
| | | GGCACTGGGAGGATGTTGAGTAAGATGGAAAACTACTGATGACCC |
| | | TTGCAGAGACAGAGTATTAGGACATGTTTGAACAGGGGCCGGGCG |
| | | ATCAGCAGGTAGCTCTAGAGGATCCCCGTCTGTCTGCACATTTCGT |
| | | AGAGCGAGTGTTCCGATACTCTAATCTCCCTAGGCAAGGTTCATAT |
| | | TTGTGTAGGTTACTTATTCTCCTTTTGTTGACTAAGTCAATAATCAG |
| | | AATCAGCAGGTTTGGAGTCAGCTTGGCAGGGATCAGCAGCCTGGG |
| | | TTGGAAGGAGGGGGTATAAAAGCCCCTTCACCAGGAGAAGCCGTC |
| | | ACACAGATCCACAAGCTCGAGCCGAGTGGTCGTGCCTCCATAGAA |
| | | GCGGCCGCCACCATGCCGGAACAAGAACGCCAGATCACAGCAAGA |
| | | GAAGGAGCAAGTAGGAAGATCCTGAGCAAGCTCTCCCTGCCCACA |
| | | AGGGCCTGGGAGCCGGCCATGAAGAAGTCTTTTGCCTTCGATAAT |
| | | GTCGGTTATGAAGGCGGGCTCGATGGCCTGGGCCCCAGCAGCCAG |
| | | CCCCAGAAGTGTTTCCTGCAGATCAAGGGCATGACCTGTGCCAGCT |
| | | GTGTTAGCAATATCGAAAGAAACCTCCAAAAAGAGGCTGGAGTTC |
| | | TGAGTGTTCTAGTGGCTTTAATGGCCGGGAAAGCCGAGATCAAGT |
| | | ATGATCCCGAGGTGATTCAGCCATTGGAGATTGCCCAGTTCATCCA |
| | | GGATCTGGGCTTCGAGGCCGCCGTGATGGAGGATTACGCCGGCTC |
| | | TGATGGCAACATCGAACTGACCATCACCGGCATGACCTGTGCTTCC |
| | | TGCGTGCATAATATCGAGAGCAAACTGACGCGGACCAATGGAATC |
| | | ACCTATGCCAGCGTCGCCCTGGCAACCTCTAAGGCTCTGGTGAAGT |
| | | TTGACCCCGAAATCATTGGACCTAGAGACATCATCAAAATTATTGA |
| | | AGAGATTGGTTTCCACGCCAGCTTAGCTCAGCGGAATCCCAACGC |
| | | ACACCATCTTGACCATAAGATGGAAATCAAACAGTGGAAGAAGAG |
| | | CTTCTTGTGTAGCCTGGTCTTCGGGATACCCGTCATGGCACTGATG |
| | | ATTTACATGCTCATTCCGTCCAACGAGCCCCACCAGTCCATGGTGC |
| | | TGGACCACAACATCATCCCTGGGCTGTCTATCCTCAACCTGATCTT |
| | | CTTTATTCTGTGCACATTTGTCCAGCTCTTGGGAGGCTGGTATTTCT |
| | | ATGTACAGGCCTACAAATCCCTGAGGCATCGAAGCGCCAACATGG |
| | | ACGTTTTAATTGTGCTGGCTACATCCATTGCCTACGTGTACTCCCTG |
| | | GTGATCTTAGTGGTTGCTGTGGCTGAAAAGGCTGAGAGAAGCCCT |
| | | GTCACCTTCTTCGACACCCCCCCCATGCTCTTCGTGTTTATTGCACT |
| | | GGGTCGCTGGCTGGAGCATTTAGCCAAGTCCAAGACATCCGAGGC |
| | | TCTGGCCAAGCTGATGTCTCTACAGGCCACGGAGGCCACCGTGGT |
| | | GACTCTGGGTGAAGACAATTTGATAATACGCGAGGAACAGGTGCC |
| | | TATGGAGCTTGTGCAGCGCGGAGATATCGTCAAGGTGGTGCCCGG |
| | | CGGCAAGTTCCCAGTGGATGGAAAAGTGTTAGAGGGCAATACCAT |
| | | GGCCGATGAATCCCTCATCACTGGCGAGGCAATGCCGGTGACCAA |
| | | GAAACCTGGAAGTACTGTCATTGCAGGCAGCATCAATGCCCATGG |
| | | GAGCGTCCTGATCAAGGCCACCCACGTGGGCAATGATACAACTCT |
| | | CGCCCAGATCGTCAAGCTAGTGGAAGAGGCTCAGATGTCTAAAGC |
| | | TCCAATCCAGCAACTGGCTGACCGCTTCAGCGGCTACTTTGTCCCC |
| | | TTTATCATCATCATGAGCACCCTCACCCTGGTGGTGTGGATAGTCA |
| | | TAGGCTTCATTGACTTTGGCGTGGTGCAGAGATACTTCCCCAACCC |
| | | TAACAAACATATCTCCCAAACCGAGGTCATTATCAGATTCGCCTTT |
| | | CAGACATCTATCACTGTGCTGTGCATAGCCTGCCCTTGCTCTCTGG |
| | | GCCTGGCCACCCCCACCGCCGTGATGGTTGGAACCGGGGTCGCTG |
| | | CACAAAATGGGATCCTGATAAAAGGGGGCAAACCCCTGGAAATGG |
| | | CACATAAGATTAAAACAGTTATGTTTGATAAGACAGGCACCATTA |
| | | CTCACGGTGTCCCTCGAGTCATGAGAGTGCTGCTGCTGGGGGACGT |
| | | GGCTACACTGCCACTGCGCAAGGTGCTGGCTGTGGTGGGCACAGC |
| | | GGAGGCCTCCTCAGAACACCCATTAGGCGTAGCTGTCACAAAATA |
| | | CTGTAAGGAGGAGCTGGGGACAGAGACCCTCGGCTATTGTACAGA |
| | | CTTCCAGGCCGTGCCTGGCTGCGGCATCGGCTGTAAGGTCAGCAAT |
| | | GTGGAGGGCATCCTGGCACACAGCGAGAGACCCCTCTCTGCCCCA |
| | | GCCTCCCACCTGAATGAAGCTGGGTCTCTGCCTGCTGAGAAGGAC |
| | | GCCGTCCCTCAGACCTTCTCCGTGTTGATTGGGAACAGAGAGTGGC |
| | | TTAGAAGGAATGGCCTGACCATCAGCTCTGACGTCTCGGATGCCAT |
| | | GACAGATCACGAGATGAAAGGCCAGACAGCCATACTGGTTGCCAT |
| | | CGACGGCGTGCTCTGTGGCATGATTGCCATCGCTGATGCTGTTAAA |
| | | CAGGAAGCTGCGCTGGCAGTCCACACCCTCCAGTCCATGGGAGTG |
| | | GACGTGGTCCTGATTACTGGAGACAACAGGAAAACCGCTCGGGCC |
| | | ATCGCTACCCAAGTGGGGATAAATAAGGTTTTTGCAGAAGTCTTAC |
| | | CCTCCCACAAAGTAGCCAAAGTGCAGGAGCTCCAGAACAAGGGTA |
| | | AAAAGGTGGCAATGGTGGGAGATGGTGTGAATGACAGCCCAGCTC |
| | | TCGCGCAGGCTGACATGGGCGTAGCCATAGGTACTGGAACAGATG |
| | | TCGCGATTGAAGCCGCTGACGTAGTCCTCATCCGAAACGATTTGCT |
| | | GGATGTGGTGGCCAGCATCCATCTGTCCAAGCGTACCGTGCGCCG |
| | | GATCCGCATCAACCTGGTCTTGGCCCTCATCTATAATCTCGTGGGC |
| | | ATCCCCATCGCTGCTGGGGTCTTCATGCCCATCGGCATTGTGCTGC |
| | | AGCCCTGGATGGGAAGCGCTGCCATGGCTGCTTCTTCTGTCTCCGT |
| | | TGTGCTGTCCAGCCTACAGCTGAAGTGCTATAAAAAGCCAGACCTC |
| | | GAGCGCTACGAAGCCCAGGCGCATGGCCATATGAAGCCCCTCACG |
| | | GCTTCCCAGGTCTCGGTTCACATTGGGATGGATGACAGATGGCGG |
| | | GACTCCCCACGCGCCACACCCTGGGACCAGGTATCCTATGTGAGCC |
| | | AGGTGAGCCTCAGCTCCCTGACATCAGATAAACCGTCTAGGCACTC |
| | | AGCTGCCGCAGATGATGACGGTGACAAATGGAGCCTCCTCCTGAA |
| | | CGGCCGCGACGAGGAGCAGTACATCTAAGAATTCGATATCAAGCT |
| | | TATCGATAATCAACCTCTGGATTACAAAATTTGTGAAAGATTGACT |
| | | GGTATTCTTAACTATGTTGCTCCTTTTACGCTATGTGGATACGCTGC |
| | | TTTAATGCCTTTGTATCATGCTATTGCTTCCCGTATGGCTTTCATTT |
| | | TCTCCTCCTTGTATAAATCCTGGTTGCTGTCTCTTTATGAGGAGTTG |
| | | TGGCCCGTTGTCAGGCAACGTGGCGTGGTGTGCACTGTGTTTGCTG |
| | | ACGCAACCCCCACTGGTTGGGGCACCTGCAGGAATAAAAGATCTT |
| | | TATTTTCATTAGATCTGTGTGTTGGTTTTTTGTGTGTCTAGAGTAGA |
| | | TAAGTAGCATGGCGGGTTAATCATTAACTACAAGGAACCCCTAGT |
| | | GATGGAGTTGGCCACTCCCTCTCTGCGCGCTCGCTCGCTCACTGAG |
| | | GCCGGGCGACCAAAGGTCGCCCGACGCCCGGGCTTTGCCCGGGCG |
| | | GCCTCAGTGAGCGAGCGAGCGCGCAGAGAGGGAGTGGCCAA |
| 66 | A13 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAATTTTAATAAATACCTCTCTGTCCTTCT |
| | | CCTAGCTTCACCCTTTGTTTGTTGAAAGGAGATACTTTAGTGCAGT |
| | | ATTGCAACAGCTTATGTAGACATGCAACCTTGAGGTCACAGACCTA |
| | | TGAGGGATTGAGACAAAGGCAAATTTAGCAAATATTTTCCAGAGT |
| | | ACTCTGCTGCAATAACGCCGCGTTCGCCCCGCCCCCGACGGTATCG |
| | | ATAAGCTTAGGAGCTTGGGCTGCAGGTCGAGGGCACTGGGAGGAT |
| | | GTTGAGTAAGATGGAAAACTACTGATGACCCTTGCAGAGACAGAG |
| | | TATTAGGACATGTTTGAACAGGGGCCGGGCGATCAGCAGGTAGCT |
| | | CTAGAGGATCCCCGTCTGTCTGCACATTTCGTAGAGCGAGTGTTCC |
| | | GATACTCTAATCTCCCTAGGCAAGGTTCATATTTGTGTAGGTTACT |
| | | TATTCTCCTTTTGTTGACTAAGTCAATAATCAGAATCAGCAGGTTT |
| | | GGAGTCAGCTTGGCAGGGATCAGCAGCCTGGGTTGGAAGGAGGGG |
| | | GTATAAAAGCCCCTTCACCAGGAGAAGCCGTCACACAGATCCACA |
| | | AGCTCGAGCCGAGTGGTCGTGCCTCCATAGAAGCGGCCGCCACCA |
| | | TGCCGGAACAAGAACGCCAGATCACAGCAAGAGAAGGAGCAAGT |
| | | AGGAAGATCCTGAGCAAGCTCTCCCTGCCCACAAGGGCCTGGGAG |
| | | CCGGCCATGAAGAAGTCTTTTGCCTTCGATAATGTCGGTTATGAAG |
| | | GCGGGCTCGATGGCCTGGGCCCCAGCAGCCAGCCCCAGAAGTGTT |
| | | TCCTGCAGATCAAGGGCATGACCTGTGCCAGCTGTGTTAGCAATAT |
| | | CGAAAGAAACCTCCAAAAAGAGGCTGGAGTTCTGAGTGTTCTAGT |
| | | GGCTTTAATGGCCGGGAAAGCCGAGATCAAGTATGATCCCGAGGT |
| | | GATTCAGCCATTGGAGATTGCCCAGTTCATCCAGGATCTGGGCTTC |
| | | GAGGCCGCCGTGATGGAGGATTACGCCGGCTCTGATGGCAACATC |
| | | GAACTGACCATCACCGGCATGACCTGTGCTTCCTGCGTGCATAATA |
| | | TCGAGAGCAAACTGACGCGGACCAATGGAATCACCTATGCCAGCG |
| | | TCGCCCTGGCAACCTCTAAGGCTCTGGTGAAGTTTGACCCCGAAAT |
| | | CATTGGACCTAGAGACATCATCAAAATTATTGAAGAGATTGGTTTC |
| | | CACGCCAGCTTAGCTCAGCGGAATCCCAACGCACACCATCTTGACC |
| | | ATAAGATGGAAATCAAACAGTGGAAGAAGAGCTTCTTGTGTAGCC |
| | | TGGTCTTCGGGATACCCGTCATGGCACTGATGATTTACATGCTCAT |
| | | TCCGTCCAACGAGCCCCACCAGTCCATGGTGCTGGACCACAACATC |
| | | ATCCCTGGGCTGTCTATCCTCAACCTGATCTTCTTTATTCTGTGCAC |
| | | ATTTGTCCAGCTCTTGGGAGGCTGGTATTTCTATGTACAGGCCTAC |
| | | AAATCCCTGAGGCATCGAAGCGCCAACATGGACGTTTTAATTGTGC |
| | | TGGCTACATCCATTGCCTACGTGTACTCCCTGGTGATCTTAGTGGTT |
| | | GCTGTGGCTGAAAAGGCTGAGAGAAGCCCTGTCACCTTCTTCGAC |
| | | ACCCCCCCCATGCTCTTCGTGTTTATTGCACTGGGTCGCTGGCTGG |
| | | AGCATTTAGCCAAGTCCAAGACATCCGAGGCTCTGGCCAAGCTGA |
| | | TGTCTCTACAGGCCACGGAGGCCACCGTGGTGACTCTGGGTGAAG |
| | | ACAATTTGATAATACGCGAGGAACAGGTGCCTATGGAGCTTGTGC |
| | | AGCGCGGAGATATCGTCAAGGTGGTGCCCGGCGGCAAGTTCCCAG |
| | | TGGATGGAAAAGTGTTAGAGGGCAATACCATGGCCGATGAATCCC |
| | | TCATCACTGGCGAGGCAATGCCGGTGACCAAGAAACCTGGAAGTA |
| | | CTGTCATTGCAGGCAGCATCAATGCCCATGGGAGCGTCCTGATCAA |
| | | GGCCACCCACGTGGGCAATGATACAACTCTCGCCCAGATCGTCAA |
| | | GCTAGTGGAAGAGGCTCAGATGTCTAAAGCTCCAATCCAGCAACT |
| | | GGCTGACCGCTTCAGCGGCTACTTTGTCCCCTTTATCATCATCATG |
| | | AGCACCCTCACCCTGGTGGTGTGGATAGTCATAGGCTTCATTGACT |
| | | TTGGCGTGGTGCAGAGATACTTCCCCAACCCTAACAAACATATCTC |
| | | CCAAACCGAGGTCATTATCAGATTCGCCTTTCAGACATCTATCACT |
| | | GTGCTGTGCATAGCCTGCCCTTGCTCTCTGGGCCTGGCCACCCCCA |
| | | CCGCCGTGATGGTTGGAACCGGGGTCGCTGCACAAAATGGGATCC |
| | | TGATAAAAGGGGGCAAACCCCTGGAAATGGCACATAAGATTAAAA |
| | | CAGTTATGTTTGATAAGACAGGCACCATTACTCACGGTGTCCCTCG |
| | | AGTCATGAGAGTGCTGCTGCTGGGGGACGTGGCTACACTGCCACT |
| | | GCGCAAGGTGCTGGCTGTGGTGGGCACAGCGGAGGCCTCCTCAGA |
| | | ACACCCATTAGGCGTAGCTGTCACAAAATACTGTAAGGAGGAGCT |
| | | GGGGACAGAGACCCTCGGCTATTGTACAGACTTCCAGGCCGTGCC |
| | | TGGCTGCGGCATCGGCTGTAAGGTCAGCAATGTGGAGGGCATCCT |
| | | GGCACACAGCGAGAGACCCCTCTCTGCCCCAGCCTCCCACCTGAAT |
| | | GAAGCTGGGTCTCTGCCTGCTGAGAAGGACGCCGTCCCTCAGACCT |
| | | TCTCCGTGTTGATTGGGAACAGAGAGTGGCTTAGAAGGAATGGCC |
| | | TGACCATCAGCTCTGACGTCTCGGATGCCATGACAGATCACGAGAT |
| | | GAAAGGCCAGACAGCCATACTGGTTGCCATCGACGGCGTGCTCTG |
| | | TGGCATGATTGCCATCGCTGATGCTGTTAAACAGGAAGCTGCGCTG |
| | | GCAGTCCACACCCTCCAGTCCATGGGAGTGGACGTGGTCCTGATTA |
| | | CTGGAGACAACAGGAAAACCGCTCGGGCCATCGCTACCCAAGTGG |
| | | GGATAAATAAGGTTTTTGCAGAAGTCTTACCCTCCCACAAAGTAGC |
| | | CAAAGTGCAGGAGCTCCAGAACAAGGGTAAAAAGGTGGCAATGG |
| | | TGGGAGATGGTGTGAATGACAGCCCAGCTCTCGCGCAGGCTGACA |
| | | TGGGCGTAGCCATAGGTACTGGAACAGATGTCGCGATTGAAGCCG |
| | | CTGACGTAGTCCTCATCCGAAACGATTTGCTGGATGTGGTGGCCAG |
| | | CATCCATCTGTCCAAGCGTACCGTGCGCCGGATCCGCATCAACCTG |
| | | GTCTTGGCCCTCATCTATAATCTCGTGGGCATCCCCATCGCTGCTG |
| | | GGGTCTTCATGCCCATCGGCATTGTGCTGCAGCCCTGGATGGGAAG |
| | | CGCTGCCATGGCTGCTTCTTCTGTCTCCGTTGTGCTGTCCAGCCTAC |
| | | AGCTGAAGTGCTATAAAAAGCCAGACCTCGAGCGCTACGAAGCCC |
| | | AGGCGCATGGCCATATGAAGCCCCTCACGGCTTCCCAGGTCTCGGT |
| | | TCACATTGGGATGGATGACAGATGGCGGGACTCCCCACGCGCCAC |
| | | ACCCTGGGACCAGGTATCCTATGTGAGCCAGGTGAGCCTCAGCTCC |
| | | CTGACATCAGATAAACCGTCTAGGCACTCAGCTGCCGCAGATGAT |
| | | GACGGTGACAAATGGAGCCTCCTCCTGAACGGCCGCGACGAGGAG |
| | | CAGTACATCTAAAATAAAAGATCTTTATTTTCATTAGATCTGTGTG |
| | | TTGGTTTTTTGTGTGTCTAGAGTAGATAAGTAGCATGGCGGGTTAA |
| | | TCATTAACTACAAGGAACCCCTAGTGATGGAGTTGGCCACTCCCTC |
| | | TCTGCGCGCTCGCTCGCTCACTGAGGCCGGGCGACCAAAGGTCGC |
| | | CCGACGCCCGGGCTTTGCCCGGGCGGCCTCAGTGAGCGAGCGAGC |
| | | GCGCAGAGAGGGAGTGGCCAA |
| 67 | A14 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCCACCATGCCGGAACA |
| | | AGAACGCCAGATCACAGCAAGAGAAGGAGCAAGTAGGAAGATCC |
| | | TGAGCAAGCTCTCCCTGCCCACAAGGGCCTGGGAGCCGGCCATGA |
| | | AGAAGTCTTTTGCCTTCGATAATGTCGGTTATGAAGGCGGGCTCGA |
| | | TGGCCTGGGCCCCAGCAGCCAGCCCCAGAAGTGTTTCCTGCAGATC |
| | | AAGGGCATGACCTGTGCCAGCTGTGTTAGCAATATCGAAAGAAAC |
| | | CTCCAAAAAGAGGCTGGAGTTCTGAGTGTTCTAGTGGCTTTAATGG |
| | | CCGGGAAAGCCGAGATCAAGTATGATCCCGAGGTGATTCAGCCAT |
| | | TGGAGATTGCCCAGTTCATCCAGGATCTGGGCTTCGAGGCCGCCGT |
| | | GATGGAGGATTACGCCGGCTCTGATGGCAACATCGAACTGACCAT |
| | | CACCGGCATGACCTGTGCTTCCTGCGTGCATAATATCGAGAGCAAA |
| | | CTGACGCGGACCAATGGAATCACCTATGCCAGCGTCGCCCTGGCA |
| | | ACCTCTAAGGCTCTGGTGAAGTTTGACCCCGAAATCATTGGACCTA |
| | | GAGACATCATCAAAATTATTGAAGAGATTGGTTTCCACGCCAGCTT |
| | | AGCTCAGCGGAATCCCAACGCACACCATCTTGACCATAAGATGGA |
| | | AATCAAACAGTGGAAGAAGAGCTTCTTGTGTAGCCTGGTCTTCGG |
| | | GATACCCGTCATGGCACTGATGATTTACATGCTCATTCCGTCCAAC |
| | | GAGCCCCACCAGTCCATGGTGCTGGACCACAACATCATCCCTGGG |
| | | CTGTCTATCCTCAACCTGATCTTCTTTATTCTGTGCACATTTGTCCA |
| | | GCTCTTGGGAGGCTGGTATTTCTATGTACAGGCCTACAAATCCCTG |
| | | AGGCATCGAAGCGCCAACATGGACGTTTTAATTGTGCTGGCTACAT |
| | | CCATTGCCTACGTGTACTCCCTGGTGATCTTAGTGGTTGCTGTGGCT |
| | | GAAAAGGCTGAGAGAAGCCCTGTCACCTTCTTCGACACCCCCCCC |
| | | ATGCTCTTCGTGTTTATTGCACTGGGTCGCTGGCTGGAGCATTTAG |
| | | CCAAGTCCAAGACATCCGAGGCTCTGGCCAAGCTGATGTCTCTACA |
| | | GGCCACGGAGGCCACCGTGGTGACTCTGGGTGAAGACAATTTGAT |
| | | AATACGCGAGGAACAGGTGCCTATGGAGCTTGTGCAGCGCGGAGA |
| | | TATCGTCAAGGTGGTGCCCGGCGGCAAGTTCCCAGTGGATGGAAA |
| | | AGTGTTAGAGGGCAATACCATGGCCGATGAATCCCTCATCACTGG |
| | | CGAGGCAATGCCGGTGACCAAGAAACCTGGAAGTACTGTCATTGC |
| | | AGGCAGCATCAATGCCCATGGGAGCGTCCTGATCAAGGCCACCCA |
| | | CGTGGGCAATGATACAACTCTCGCCCAGATCGTCAAGCTAGTGGA |
| | | AGAGGCTCAGATGTCTAAAGCTCCAATCCAGCAACTGGCTGACCG |
| | | CTTCAGCGGCTACTTTGTCCCCTTTATCATCATCATGAGCACCCTCA |
| | | CCCTGGTGGTGTGGATAGTCATAGGCTTCATTGACTTTGGCGTGGT |
| | | GCAGAGATACTTCCCCAACCCTAACAAACATATCTCCCAAACCGA |
| | | GGTCATTATCAGATTCGCCTTTCAGACATCTATCACTGTGCTGTGC |
| | | ATAGCCTGCCCTTGCTCTCTGGGCCTGGCCACCCCCACCGCCGTGA |
| | | TGGTTGGAACCGGGGTCGCTGCACAAAATGGGATCCTGATAAAAG |
| | | GGGGCAAACCCCTGGAAATGGCACATAAGATTAAAACAGTTATGT |
| | | TTGATAAGACAGGCACCATTACTCACGGTGTCCCTCGAGTCATGAG |
| | | AGTGCTGCTGCTGGGGGACGTGGCTACACTGCCACTGCGCAAGGT |
| | | GCTGGCTGTGGTGGGCACAGCGGAGGCCTCCTCAGAACACCCATT |
| | | AGGCGTAGCTGTCACAAAATACTGTAAGGAGGAGCTGGGGACAGA |
| | | GACCCTCGGCTATTGTACAGACTTCCAGGCCGTGCCTGGCTGCGGC |
| | | ATCGGCTGTAAGGTCAGCAATGTGGAGGGCATCCTGGCACACAGC |
| | | GAGAGACCCCTCTCTGCCCCAGCCTCCCACCTGAATGAAGCTGGGT |
| | | CTCTGCCTGCTGAGAAGGACGCCGTCCCTCAGACCTTCTCCGTGTT |
| | | GATTGGGAACAGAGAGTGGCTTAGAAGGAATGGCCTGACCATCAG |
| | | CTCTGACGTCTCGGATGCCATGACAGATCACGAGATGAAAGGCCA |
| | | GACAGCCATACTGGTTGCCATCGACGGCGTGCTCTGTGGCATGATT |
| | | GCCATCGCTGATGCTGTTAAACAGGAAGCTGCGCTGGCAGTCCAC |
| | | ACCCTCCAGTCCATGGGAGTGGACGTGGTCCTGATTACTGGAGAC |
| | | AACAGGAAAACCGCTCGGGCCATCGCTACCCAAGTGGGGATAAAT |
| | | AAGGTTTTTGCAGAAGTCTTACCCTCCCACAAAGTAGCCAAAGTGC |
| | | AGGAGCTCCAGAACAAGGGTAAAAAGGTGGCAATGGTGGGAGAT |
| | | GGTGTGAATGACAGCCCAGCTCTCGCGCAGGCTGACATGGGCGTA |
| | | GCCATAGGTACTGGAACAGATGTCGCGATTGAAGCCGCTGACGTA |
| | | GTCCTCATCCGAAACGATTTGCTGGATGTGGTGGCCAGCATCCATC |
| | | TGTCCAAGCGTACCGTGCGCCGGATCCGCATCAACCTGGTCTTGGC |
| | | CCTCATCTATAATCTCGTGGGCATCCCCATCGCTGCTGGGGTCTTC |
| | | ATGCCCATCGGCATTGTGCTGCAGCCCTGGATGGGAAGCGCTGCC |
| | | ATGGCTGCTTCTTCTGTCTCCGTTGTGCTGTCCAGCCTACAGCTGAA |
| | | GTGCTATAAAAAGCCAGACCTCGAGCGCTACGAAGCCCAGGCGCA |
| | | TGGCCATATGAAGCCCCTCACGGCTTCCCAGGTCTCGGTTCACATT |
| | | GGGATGGATGACAGATGGCGGGACTCCCCACGCGCCACACCCTGG |
| | | GACCAGGTATCCTATGTGAGCCAGGTGAGCCTCAGCTCCCTGACAT |
| | | CAGATAAACCGTCTAGGCACTCAGCTGCCGCAGATGATGACGGTG |
| | | ACAAATGGAGCCTCCTCCTGAACGGCCGCGACGAGGAGCAGTACA |
| | | TCTAAAATAAAAGATCTTTATTTTCATTAGATCTGTGTGTTGGTTTT |
| | | TTGTGTGTCTAGAGTAGATAAGTAGCATGGCGGGTTAATCATTAAC |
| | | TACAAGGAACCCCTAGTGATGGAGTTGGCCACTCCCTCTCTGCGCG |
| | | CTCGCTCGCTCACTGAGGCCGGGCGACCAAAGGTCGCCCGACGCC |
| | | CGGGCTTTGCCCGGGCGGCCTCAGTGAGCGAGCGAGCGCGCAGAG |
| | | AGGGAGTGGCCAA |
| 68 | A15 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCCACCATGCCTGAGCA |
| | | GGAGAGACAGATCACAGCCAGAGAAGGGGCCAGTCGGAAAATCT |
| | | TATCTAAGCTTTCTTTGCCTACCCGTGCCTGGGAACCAGCAATGAA |
| | | GAAGAGTTTTGCTTTTGACAATGTTGGCTATGAAGGTGGTCTGGAT |
| | | GGCCTGGGCCCTTCTTCTCAGGTGGCCCCTGATGGAGCCGAAGGG |
| | | AGTGGGACAGATCACAGGTCTTCCAGTTCTCATTCCCCTGGCTCCC |
| | | CACCGAGAAACCAGGTCCAGGGCACATGCAGTAAGTGCTTCTTAC |
| | | AGATCAAAGGCATGACCTGTGCATCCTGTGTGTCTAACATAGAAA |
| | | GGAATCTGCAGAAAGAAGCTGGTGTTCTCTCCGTGTTGGTTGCCTT |
| | | GATGGCAGGAAAGGCAGAGATCAAGTATGACCCAGAGGTCATCCA |
| | | GCCCCTCGAGATAGCTCAGTTCATCCAGGACCTGGGTTTTGAGGCA |
| | | GCAGTCATGGAGGACTACGCAGGCTCCGATGGCAACATTGAGCTG |
| | | ACAATCACAGGGATGACCTGCGCGTCCTGTGTCCACAACATAGAG |
| | | TCCAAACTCACGAGGACAAATGGCATCACTTATGCCTCCGTTGCCC |
| | | TTGCCACCAGCAAAGCCCTTGTTAAGTTTGACCCGGAAATTATCGG |
| | | TCCACGGGATATTATCAAAATTATTGAGGAAATTGGCTTTCATGCT |
| | | TCCCTGGCCCAGAGAAACCCCAACGCTCATCACTTGGACCACAAG |
| | | ATGGAAATAAAGCAGTGGAAGAAGTCTTTTCTTGTGAGTCTGTTTT |
| | | TCTGTATTCCTGTAATGGGGCTGATGATATATATGATGGTTATGGA |
| | | CCACCACTTTGCAACTCTTCACCATAATCAAAACATGAGTAAAGAA |
| | | GAAATGATCAACCTTCATTCTTCTATGTTCCTGGAGCGCCAGATTC |
| | | TTCCAGGATTGTCTGTTATGAATTTGCTGTCCTTTTTATTGTGTGTA |
| | | CCTGTACAGTTTTTCGGAGGCTGGTACTTCTACATTCAGGCTTATA |
| | | AAGCACTGAAGCATAAGACAGCAAATATGGACGTACTGATTGTGC |
| | | TGGCAACCACCATTGCATTTGCCTACTCTTTGATTATTCTTCTAGTT |
| | | GCAATGTATGAGAGAGCCAAAGTGAACCCTATTACTTTCTTTGACA |
| | | CACCCCCTATGCTGTTTGTGTTTATTGCACTAGGCCGATGGCTGGA |
| | | ACATATAGCAAAGGGCAAAACATCAGAGGCTCTTGCAAAGTTAAT |
| | | TTCACTACAAGCTACAGAAGCAACTATTGTAACTCTTGATTCTGAT |
| | | AATATCCTCCTCAGTGAAGAACAAGTGGATGTGGAACTTGTACAA |
| | | CGTGGAGATATCATTAAAGTAGTTCCAGGAGGCAAATTTCCAGTG |
| | | GATGGTCGTGTTATTGAAGGACATTCTATGGTAGATGAGTCCCTCA |
| | | TCACAGGGGAGGCAATGCCTGTGGCTAAGAAACCTGGCAGCACAG |
| | | TGATTGCTGGTTCCATTAACCAGAACGGGTCACTGCTTATCTGCGC |
| | | AACACATGTTGGAGCAGACACAACCCTTTCTCAAATTGTCAAACTT |
| | | GTGGAAGAGGCACAAACATCAAAGGCTCCTATCCAGCAGTTTGCA |
| | | GACAAACTCAGTGGCTATTTTGTTCCTTTTATTGTTTTTGTTTCCAT |
| | | TGCCACCCTCTTGGTATGGATTGTAATTGGATTTCTGAATTTTGAA |
| | | ATTGTGGAAACCTACTTTCCTGGCTACAATAGAAGTATCTCCCGAA |
| | | CAGAAACGATAATACGATTTGCTTTCCAAGCCTCTATCACAGTTCT |
| | | GTGTATTGCATGTCCCTGTTCACTGGGACTGGCCACTCCAACTGCT |
| | | GTGATGGTGGGTACAGGAGTAGGTGCTCAAAATGGCATACTAATA |
| | | AAAGGTGGAGAGCCATTGGAGATGGCTCATAAGGTAAAGGTAGTG |
| | | GTATTTGATAAGACTGGAACCATTACTCACGGAACCCCAGTGGTG |
| | | AATCAAGTAAAGGTTCTAACTGAAAGTAACAGAATATCACACCAT |
| | | AAAATCTTGGCCATTGTGGGAACTGCTGAAAGTAACAGTGAACAC |
| | | CCTCTAGGAACAGCCATAACCAAATATTGCAAACAGGAGCTGGAC |
| | | ACTGAAACCTTGGGTACCTGCATAGATTTCCAGGTTGTGCCAGGCT |
| | | GTGGTATTAGCTGTAAAGTCACCAATATTGAAGGCTTGCTACATAA |
| | | GAATAACTGGAATATAGAGGACAATAATATTAAAAATGCATCCCT |
| | | GGTTCAAATTGATGCCAGTAATGAACAGTCATCAACTTCGTCTTCC |
| | | ATGATTATTGATGCCCAGATCTCAAATGCTCTTAATGCTCAGCAGT |
| | | ATAAAGTCCTCATTGGTAACCGGGAGTGGATGATTAGAAATGGTC |
| | | TTGTCATTAATAACGATGTAAATGATTTCATGACTGAACATGAGAG |
| | | AAAAGGTCGGACTGCTGTATTAGTAGCAGTTGATGATGAGCTGTGT |
| | | GGCTTGATAGCCATTGCAGACACAGTGAAGCCTGAAGCAGAACTG |
| | | GCTATCCATATTCTGAAATCTATGGGCTTAGAAGTAGTTCTGATGA |
| | | CTGGAGACAACAGTAAAACAGCTAGATCTATTGCTTCTCAGGTTGG |
| | | CATTACTAAGGTGTTTGCTGAAGTTCTACCTTCTCACAAGGTTGCT |
| | | AAAGTGAAGCAACTTCAAGAGGAGGGGAAACGGGTAGCAATGGT |
| | | GGGAGATGGAATCAATGACTCCCCAGCTCTGGCAATGGCTAATGT |
| | | GGGAATTGCTATTGGCACAGGCACAGATGTAGCCATTGAAGCAGC |
| | | TGATGTGGTTTTGATAAGGAATGATCTTCTGGATGTAGTGGCAAGT |
| | | ATTGACTTATCAAGAAAGACAGTCAAGAGGATTCGGATAAATTTT |
| | | GTCTTTGCTCTAATTTATAATCTGGTTGGAATTCCCATAGCTGCTGG |
| | | AGTTTTTATGCCCATTGGTTTGGTTTTGCAGCCCTGGATGGGATCTG |
| | | CAGCAATGGCTGCTTCATCTGTTTCTGTAGTACTTTCTTCTCTCCAG |
| | | CTCAAGTGCTATAAGAAGCCTGACCTGGAGAGGTATGAGGCACAG |
| | | GCGCATGGCCACATGAAGCCCCTGACGGCATCCCAGGTCAGTGTG |
| | | CACATAGGCATGGATGACAGGTGGCGGGACTCCCCCAGGGCCACA |
| | | CCATGGGACCAGGTCAGCTATGTCAGCCAGGTGTCGCTGTCCTCCC |
| | | TGACGTCCGACAAGCCATCTCGGCACAGCGCTGCAGCAGACGATG |
| | | ATGGGGACAAGTGGTCTCTGCTCCTGAATGGCAGGGATGAGGAGC |
| | | AGTACATCTGAGAATTCGATATCAAGCTTATCGATAATCAACCTCT |
| | | GGATTACAAAATTTGTGAAAGATTGACTGGTATTCTTAACTATGTT |
| | | GCTCCTTTTACGCTATGTGGATACGCTGCTTTAATGCCTTTGTATCA |
| | | TGCTATTGCTTCCCGTATGGCTTTCATTTTCTCCTCCTTGTATAAAT |
| | | CCTGGTTGCTGTCTCTTTATGAGGAGTTGTGGCCCGTTGTCAGGCA |
| | | ACGTGGCGTGGTGTGCACTGTGTTTGCTGACGCAACCCCCACTGGT |
| | | TGGGGCACCTGCAGGAATAAAAGATCTTTATTTTCATTAGATCTGT |
| | | GTGTTGGTTTTTTGTGTGTCTAGAGTAGATAAGTAGCATGGCGGGT |
| | | TAATCATTAACTACAAGGAACCCCTAGTGATGGAGTTGGCCACTCC |
| | | CTCTCTGCGCGCTCGCTCGCTCACTGAGGCCGGGCGACCAAAGGTC |
| | | GCCCGACGCCCGGGCTTTGCCCGGGCGGCCTCAGTGAGCGAGCGA |
| | | GCGCGCAGAGAGGGAGTGGCCAA |
| 69 | A16 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCCACCATGCCTGAGCA |
| | | GGAGAGACAGATCACAGCCAGAGAAGGGGCCAGTCGGAAAATCT |
| | | TATCTAAGCTTTCTTTGCCTACCCGTGCCTGGGAACCAGCAATGAA |
| | | GAAGAGTTTTGCTTTTGACAATGTTGGCTATGAAGGTGGTCTGGAT |
| | | GGCCTGGGCCCTTCTTCTCAGGTGGCCCCTGATGGAGCCGAAGGG |
| | | AGTGGGACAGATCACAGGTCTTCCAGTTCTCATTCCCCTGGCTCCC |
| | | CACCGAGAAACCAGGTCCAGGGCACATGCAGTAAGTGCTTCTTAC |
| | | AGATCAAAGGCATGACCTGTGCATCCTGTGTGTCTAACATAGAAA |
| | | GGAATCTGCAGAAAGAAGCTGGTGTTCTCTCCGTGTTGGTTGCCTT |
| | | GATGGCAGGAAAGGCAGAGATCAAGTATGACCCAGAGGTCATCCA |
| | | GCCCCTCGAGATAGCTCAGTTCATCCAGGACCTGGGTTTTGAGGCA |
| | | GCAGTCATGGAGGACTACGCAGGCTCCGATGGCAACATTGAGCTG |
| | | ACAATCACAGGGATGACCTGCGCGTCCTGTGTCCACAACATAGAG |
| | | TCCAAACTCACGAGGACAAATGGCATCACTTATGCCTCCGTTGCCC |
| | | TTGCCACCAGCAAAGCCCTTGTTAAGTTTGACCCGGAAATTATCGG |
| | | TCCACGGGATATTATCAAAATTATTGAGGAAATTGGCTTTCATGCT |
| | | TCCCTGGCCCAGAGAAACCCCAACGCTCATCACTTGGACCACAAG |
| | | ATGGAAATAAAGCAGTGGAAGAAGTCTTTTCTTGTGAGTCTGTTTT |
| | | TCTGTATTCCTGTAATGGGGCTGATGATATATATGATGGTTATGGA |
| | | CCACCACTTTGCAACTCTTCACCATAATCAAAACATGAGTAAAGAA |
| | | GAAATGATCAACCTTCATTCTTCTATGTTCCTGGAGCGCCAGATTC |
| | | TTCCAGGATTGTCTGTTATGAATTTGCTGTCCTTTTTATTGTGTGTA |
| | | CCTGTACAGTTTTTCGGAGGCTGGTACTTCTACATTCAGGCTTATA |
| | | AAGCACTGAAGCATAAGACAGCAAATATGGACGTACTGATTGTGC |
| | | TGGCAACCACCATTGCATTTGCCTACTCTTTGATTATTCTTCTAGTT |
| | | GCAATGTATGAGAGAGCCAAAGTGAACCCTATTACTTTCTTTGACA |
| | | CACCCCCTATGCTGTTTGTGTTTATTGCACTAGGCCGATGGCTGGA |
| | | ACATATAGCAAAGGGCAAAACATCAGAGGCTCTTGCAAAGTTAAT |
| | | TTCACTACAAGCTACAGAAGCAACTATTGTAACTCTTGATTCTGAT |
| | | AATATCCTCCTCAGTGAAGAACAAGTGGATGTGGAACTTGTACAA |
| | | CGTGGAGATATCATTAAAGTAGTTCCAGGAGGCAAATTTCCAGTG |
| | | GATGGTCGTGTTATTGAAGGACATTCTATGGTAGATGAGTCCCTCA |
| | | TCACAGGGGAGGCAATGCCTGTGGCTAAGAAACCTGGCAGCACAG |
| | | TGATTGCTGGTTCCATTAACCAGAACGGGTCACTGCTTATCTGCGC |
| | | AACACATGTTGGAGCAGACACAACCCTTTCTCAAATTGTCAAACTT |
| | | GTGGAAGAGGCACAAACATCAAAGGCTCCTATCCAGCAGTTTGCA |
| | | GACAAACTCAGTGGCTATTTTGTTCCTTTTATTGTTTTTGTTTCCAT |
| | | TGCCACCCTCTTGGTATGGATTGTAATTGGATTTCTGAATTTTGAA |
| | | ATTGTGGAAACCTACTTTCCTGGCTACAATAGAAGTATCTCCCGAA |
| | | CAGAAACGATAATACGATTTGCTTTCCAAGCCTCTATCACAGTTCT |
| | | GTGTATTGCATGTCCCTGTTCACTGGGACTGGCCACTCCAACTGCT |
| | | GTGATGGTGGGTACAGGAGTAGGTGCTCAAAATGGCATACTAATA |
| | | AAAGGTGGAGAGCCATTGGAGATGGCTCATAAGGTAAAGGTAGTG |
| | | GTATTTGATAAGACTGGAACCATTACTCACGGAACCCCAGTGGTG |
| | | AATCAAGTAAAGGTTCTAACTGAAAGTAACAGAATATCACACCAT |
| | | AAAATCTTGGCCATTGTGGGAACTGCTGAAAGTAACAGTGAACAC |
| | | CCTCTAGGAACAGCCATAACCAAATATTGCAAACAGGAGCTGGAC |
| | | ACTGAAACCTTGGGTACCTGCATAGATTTCCAGGTTGTGCCAGGCT |
| | | GTGGTATTAGCTGTAAAGTCACCAATATTGAAGGCTTGCTACATAA |
| | | GAATAACTGGAATATAGAGGACAATAATATTAAAAATGCATCCCT |
| | | GGTTCAAATTGATGCCAGTAATGAACAGTCATCAACTTCGTCTTCC |
| | | ATGATTATTGATGCCCAGATCTCAAATGCTCTTAATGCTCAGCAGT |
| | | ATAAAGTCCTCATTGGTAACCGGGAGTGGATGATTAGAAATGGTC |
| | | TTGTCATTAATAACGATGTAAATGATTTCATGACTGAACATGAGAG |
| | | AAAAGGTCGGACTGCTGTATTAGTAGCAGTTGATGATGAGCTGTGT |
| | | GGCTTGATAGCCATTGCAGACACAGTGAAGCCTGAAGCAGAACTG |
| | | GCTATCCATATTCTGAAATCTATGGGCTTAGAAGTAGTTCTGATGA |
| | | CTGGAGACAACAGTAAAACAGCTAGATCTATTGCTTCTCAGGTTGG |
| | | CATTACTAAGGTGTTTGCTGAAGTTCTACCTTCTCACAAGGTTGCT |
| | | AAAGTGAAGCAACTTCAAGAGGAGGGGAAACGGGTAGCAATGGT |
| | | GGGAGATGGAATCAATGACTCCCCAGCTCTGGCAATGGCTAATGT |
| | | GGGAATTGCTATTGGCACAGGCACAGATGTAGCCATTGAAGCAGC |
| | | TGATGTGGTTTTGATAAGGAATGATCTTCTGGATGTAGTGGCAAGT |
| | | ATTGACTTATCAAGAAAGACAGTCAAGAGGATTCGGATAAATTTT |
| | | GTCTTTGCTCTAATTTATAATCTGGTTGGAATTCCCATAGCTGCTGG |
| | | AGTTTTTATGCCCATTGGTTTGGTTTTGCAGCCCTGGATGGGATCTG |
| | | CAGCAATGGCTGCTTCATCTGTTTCTGTAGTACTTTCTTCTCTCCAG |
| | | CTCAAGTGCTATAAGAAGCCTGACCTGGAGAGGTATGAGGCACAG |
| | | GCGCATGGCCACATGAAGCCCCTGACGGCATCCCAGGTCAGTGTG |
| | | CACATAGGCATGGATGACAGGTGGCGGGACTCCCCCAGGGCCACA |
| | | CCATGGGACCAGGTCAGCTATGTCAGCCAGGTGTCGCTGTCCTCCC |
| | | TGACGTCCGACAAGCCATCTCGGCACAGCGCTGCAGCAGACGATG |
| | | ATGGGGACAAGTGGTCTCTGCTCCTGAATGGCAGGGATGAGGAGC |
| | | AGTACATCTGAAATAAAAGATCTTTATTTTCATTAGATCTGTGTGT |
| | | TGGTTTTTTGTGTGTCTAGAGTAGATAAGTAGCATGGCGGGTTAAT |
| | | CATTAACTACAAGGAACCCCTAGTGATGGAGTTGGCCACTCCCTCT |
| | | CTGCGCGCTCGCTCGCTCACTGAGGCCGGGCGACCAAAGGTCGCC |
| | | CGACGCCCGGGCTTTGCCCGGGCGGCCTCAGTGAGCGAGCGAGCG |
| | | CGCAGAGAGGGAGTGGCCAA |
| 70 | A17 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCCACCATGCCTGAACA |
| | | AGAAAGACAGATCACAGCAAGAGAAGGAGCATCTAGGAAGATCC |
| | | TGAGCAAGCTCTCCCTGCCCACAAGGGCCTGGGAGCCTGCCATGA |
| | | AGAAGTCTTTTGCCTTTGATAATGTGGGTTATGAAGGAGGGCTGGA |
| | | TGGCCTGGGCCCCAGCAGCCAGGTGGCAACCACCCTGATTGCCATT |
| | | GCAGGCATGACCTGTGCATCTTGTGTGCATAGCATTGAAGGCATGA |
| | | TTAGCCAGCTGGAGGGAGTGCAGCAGATCTCTGTCTCCCTGGCTGA |
| | | GGGGACAGCCACTGTCCTGTACAATCCCTCTGTTATTTCCCCTGAG |
| | | GAGCTGAGAGCTGCCATTGAGGACATGGGATTTGAAGCATCTGTG |
| | | GTGTCTGAGTCCTGCTCCACCAATCCCCTGGGAAATCACTCTGCTG |
| | | GCAATTCTATGGTTCAGACCACAGATGGGACCCCAACCTCTGTGCA |
| | | GGAAGTGGCACCCCATACTGGCAGACTGCCTGCCAATCATGCTCCT |
| | | GATATCCTGGCCAAGAGCCCCCAGAGCACCAGGGCTGTGGCACCC |
| | | CAGAAGTGTTTCCTGCAGATCAAGGGCATGACCTGTGCCAGCTGTG |
| | | TTAGCAATATTGAAAGAAACCTCCAAAAAGAGGCTGGAGTTCTGT |
| | | CTGTTCTGGTGGCTCTGATGGCTGGGAAAGCAGAGATCAAGTATG |
| | | ATCCAGAGGTGATTCAGCCACTGGAGATTGCCCAGTTCATCCAGG |
| | | ATCTGGGCTTTGAGGCAGCTGTGATGGAGGATTATGCTGGCTCTGA |
| | | TGGCAACATTGAACTGACCATCACAGGCATGACCTGTGCTTCCTGT |
| | | GTGCATAATATTGAGAGCAAACTGACCAGAACCAATGGAATCACC |
| | | TATGCCTCTGTTGCCCTGGCAACCTCTAAGGCTCTGGTGAAGTTTG |
| | | ACCCTGAAATCATTGGACCTAGAGACATCATCAAAATTATTGAAG |
| | | AGATTGGTTTCCATGCCAGCCTGGCTCAGAGAAATCCCAATGCACA |
| | | CCATCTGGACCATAAGATGGAAATCAAACAGTGGAAGAAGAGCTT |
| | | CCTGTGTAGCCTGGTCTTTGGGATTCCTGTCATGGCACTGATGATTT |
| | | ACATGCTCATTCCCTCCAATGAGCCCCACCAGTCCATGGTGCTGGA |
| | | CCACAACATCATCCCTGGGCTGTCTATCCTCAACCTGATCTTCTTTA |
| | | TTCTGTGCACATTTGTCCAGCTCCTGGGAGGCTGGTATTTCTATGTT |
| | | CAGGCCTACAAATCCCTGAGGCATAGATCTGCCAACATGGATGTTC |
| | | TGATTGTGCTGGCTACATCCATTGCCTATGTGTACTCCCTGGTGATC |
| | | CTGGTGGTTGCTGTGGCTGAAAAGGCTGAGAGAAGCCCTGTCACC |
| | | TTCTTTGACACCCCCCCCATGCTCTTTGTGTTTATTGCACTGGGTAG |
| | | GTGGCTGGAGCATCTGGCCAAGTCCAAGACATCTGAGGCTCTGGC |
| | | CAAGCTGATGTCTCTCCAGGCCACTGAGGCCACTGTGGTGACTCTG |
| | | GGTGAAGACAATCTGATCATTAGAGAGGAACAGGTGCCTATGGAG |
| | | CTGGTGCAGAGAGGAGATATTGTCAAGGTGGTGCCAGGAGGCAAG |
| | | TTCCCAGTGGATGGAAAAGTGCTGGAGGGCAATACCATGGCTGAT |
| | | GAATCCCTCATCACTGGGGAGGCAATGCCTGTGACCAAGAAACCT |
| | | GGAAGCACTGTCATTGCAGGCAGCATCAATGCCCATGGGTCTGTCC |
| | | TGATCAAGGCCACCCATGTGGGCAATGATACAACTCTGGCCCAGA |
| | | TTGTCAAGCTGGTGGAAGAGGCTCAGATGTCTAAAGCTCCAATCC |
| | | AGCAACTGGCTGACAGATTCTCTGGCTACTTTGTCCCCTTTATCATC |
| | | ATCATGAGCACCCTCACCCTGGTGGTGTGGATTGTCATTGGCTTCA |
| | | TTGACTTTGGGGTGGTGCAGAGATACTTCCCCAACCCTAACAAACA |
| | | TATCTCCCAAACAGAGGTCATTATCAGATTTGCCTTTCAGACATCT |
| | | ATCACTGTGCTGTGCATTGCCTGCCCTTGCTCTCTGGGCCTGGCCA |
| | | CCCCCACTGCTGTGATGGTTGGAACAGGGGTGGCTGCACAAAATG |
| | | GGATCCTGATCAAAGGGGGCAAACCCCTGGAAATGGCACATAAGA |
| | | TTAAAACAGTTATGTTTGATAAGACAGGCACCATTACTCATGGTGT |
| | | CCCTAGGGTCATGAGAGTGCTGCTGCTGGGGGATGTGGCTACACT |
| | | GCCACTGAGGAAGGTGCTGGCTGTGGTGGGCACAGCTGAGGCCTC |
| | | CTCTGAACACCCACTGGGAGTTGCTGTCACAAAATACTGTAAGGA |
| | | GGAGCTGGGGACAGAGACCCTGGGCTATTGTACAGACTTCCAGGC |
| | | TGTGCCTGGCTGTGGCATTGGCTGTAAGGTCAGCAATGTGGAGGG |
| | | CATCCTGGCACACTCTGAGAGACCCCTCTCTGCCCCAGCCTCCCAC |
| | | CTGAATGAAGCTGGGTCTCTGCCTGCTGAGAAGGATGCTGTCCCTC |
| | | AGACCTTCTCTGTGCTGATTGGGAACAGAGAGTGGCTGAGAAGGA |
| | | ATGGCCTGACCATCAGCTCTGATGTCTCTGATGCCATGACAGATCA |
| | | TGAGATGAAAGGCCAGACAGCCATTCTGGTTGCCATTGATGGTGT |
| | | GCTCTGTGGCATGATTGCCATTGCTGATGCTGTTAAACAGGAAGCT |
| | | GCACTGGCAGTCCACACCCTCCAGTCCATGGGAGTGGATGTGGTCC |
| | | TGATTACTGGAGACAACAGGAAAACAGCTAGGGCCATTGCTACCC |
| | | AAGTGGGGATCAATAAGGTTTTTGCAGAAGTCCTCCCCTCCCACAA |
| | | AGTGGCCAAAGTGCAGGAGCTCCAGAACAAGGGTAAAAAGGTGG |
| | | CAATGGTGGGAGATGGTGTGAATGACAGCCCAGCTCTGGCCCAGG |
| | | CTGACATGGGAGTGGCCATTGGTACTGGAACAGATGTGGCCATTG |
| | | AAGCAGCTGATGTTGTCCTCATCAGGAATGATCTCCTGGATGTGGT |
| | | GGCCAGCATCCATCTGTCCAAGAGGACTGTGAGGAGAATCAGGAT |
| | | CAACCTGGTCCTGGCCCTCATCTATAATCTGGTGGGCATCCCCATT |
| | | GCTGCTGGGGTCTTCATGCCCATTGGCATTGTGCTGCAGCCCTGGA |
| | | TGGGATCTGCTGCCATGGCTGCTTCTTCTGTCTCTGTTGTGCTGTCC |
| | | AGCCTGCAGCTGAAGTGCTATAAAAAGCCAGACCTGGAGAGATAT |
| | | GAAGCCCAGGCCCATGGCCATATGAAGCCCCTCACAGCTTCCCAG |
| | | GTCTCTGTTCACATTGGGATGGATGACAGATGGAGGGACTCCCCA |
| | | AGGGCCACACCCTGGGACCAGGTGTCCTATGTGAGCCAGGTGAGC |
| | | CTCAGCTCCCTGACATCTGATAAACCCTCTAGGCACTCTGCTGCTG |
| | | CAGATGATGATGGTGACAAATGGAGCCTCCTCCTGAATGGCAGAG |
| | | ATGAGGAGCAGTACATCTAAGAATTCGATATCAAGCTTATCGATA |
| | | ATCAACCTCTGGATTACAAAATTTGTGAAAGATTGACTGGTATTCT |
| | | TAACTATGTTGCTCCTTTTACGCTATGTGGATACGCTGCTTTAATGC |
| | | CTTTGTATCATGCTATTGCTTCCCGTATGGCTTTCATTTTCTCCTCCT |
| | | TGTATAAATCCTGGTTGCTGTCTCTTTATGAGGAGTTGTGGCCCGTT |
| | | GTCAGGCAACGTGGCGTGGTGTGCACTGTGTTTGCTGACGCAACCC |
| | | CCACTGGTTGGGGCACCTGCAGGAATAAAAGATCTTTATTTTCATT |
| | | AGATCTGTGTGTTGGTTTTTTGTGTGTCTAGAGTAGATAAGTAGCA |
| | | TGGCGGGTTAATCATTAACTACAAGGAACCCCTAGTGATGGAGTT |
| | | GGCCACTCCCTCTCTGCGCGCTCGCTCGCTCACTGAGGCCGGGCGA |
| | | CCAAAGGTCGCCCGACGCCCGGGCTTTGCCCGGGCGGCCTCAGTG |
| | | AGCGAGCGAGCGCGCAGAGAGGGAGTGGCCAA |
| 71 | A18 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTAATAATTACCAGCGCGGGCCAAAT |
| | | AAATAATCCGCGAGGGGCAGCTGGACTTTGGACTCCGCGCGCTGG |
| | | TAATTATTAACCTCGCGAATATTGATTCGAGGCCGCGATTGCCGCA |
| | | ATCGCGAGGGGCAGGTGACCTTTGCCCAGCGCGCGTTCGCCCCGC |
| | | CCCGGACGGTATCGATAAGCTTAGGAGCTTGGGCTGCAGGTCGAG |
| | | GGCACTGGGAGGATGTTGAGTAAGATGGAAAACTACTGATGACCC |
| | | TTGCAGAGACAGAGTATTAGGACATGTTTGAACAGGGGCCGGGCG |
| | | ATCAGCAGGTAGCTCTAGAGGATCCCCGTCTGTCTGCACATTTCGT |
| | | AGAGCGAGTGTTCCGATACTCTAATCTCCCTAGGCAAGGTTCATAT |
| | | TTGTGTAGGTTACTTATTCTCCTTTTGTTGACTAAGTCAATAATCAG |
| | | AATCAGCAGGTTTGGAGTCAGCTTGGCAGGGATCAGCAGCCTGGG |
| | | TTGGAAGGAGGGGGTATAAAAGCCCCTTCACCAGGAGAAGCCGTC |
| | | ACACAGATCCACAAGCTCGAGCCGAGTGGTCGTGCCTCCATAGAA |
| | | GCGGCCGCAACTCCTAAAAAACCGCCACCATGGCGGCGCCGGAAC |
| | | AAGAACGCCAGATCACAGCAAGAGAAGGAGCAAGTAGGAAGATC |
| | | CTGAGCAAGCTCTCCCTGCCCACAAGGGCCTGGGAGCCGGCCATG |
| | | AAGAAGTCTTTTGCCTTCGATAATGTCGGTTATGAAGGCGGGCTCG |
| | | ATGGCCTGGGCCCCAGCAGCCAGGTGGCCAAGTGTTTCCTGCAGA |
| | | TCAAGGGCATGACCTGTGCCAGCTGTGTTAGCAATATCGAAAGAA |
| | | ACCTCCAAAAAGAGGCTGGAGTTCTGAGTGTTCTAGTGGCTTTAAT |
| | | GGCCGGGAAAGCCGAGATCAAGTATGATCCCGAGGTGATTCAGCC |
| | | ATTGGAGATTGCCCAGTTCATCCAGGATCTGGGCTTCGAGGCCGCC |
| | | GTGATGGAGGATTACGCCGGCTCTGATGGCAACATCGAACTGACC |
| | | ATCACCGGCATGACCTGTGCTTCCTGCGTGCATAATATCGAGAGCA |
| | | AACTGACGCGGACCAATGGAATCACCTATGCCAGCGTCGCCCTGG |
| | | CAACCTCTAAGGCTCTGGTGAAGTTTGACCCCGAAATCATTGGACC |
| | | TAGAGACATCATCAAAATTATTGAAGAGATTGGTTTCCACGCCAGC |
| | | TTAGCTCAGCGGAATCCCAACGCACACCATCTTGACCATAAGATG |
| | | GAAATCAAACAGTGGAAGAAGAGCTTCTTGTGTAGCCTGGTCTTC |
| | | GGGATACCCGTCATGGCACTGATGATTTACATGCTCATTCCGTCCA |
| | | ACGAGCCCCACCAGTCCATGGTGCTGGACCACAACATCATCCCTG |
| | | GGCTGTCTATCCTCAACCTGATCTTCTTTATTCTGTGCACATTTGTC |
| | | CAGCTCTTGGGAGGCTGGTATTTCTATGTACAGGCCTACAAATCCC |
| | | TGAGGCATCGAAGCGCCAACATGGACGTTTTAATTGTGCTGGCTAC |
| | | ATCCATTGCCTACGTGTACTCCCTGGTGATCTTAGTGGTTGCTGTG |
| | | GCTGAAAAGGCTGAGAGAAGCCCTGTCACCTTCTTCGACACCCCCC |
| | | CCATGCTCTTCGTGTTTATTGCACTGGGTCGCTGGCTGGAGCATTT |
| | | AGCCAAGTCCAAGACATCCGAGGCTCTGGCCAAGCTGATGTCTCT |
| | | ACAGGCCACGGAGGCCACCGTGGTGACTCTGGGTGAAGACAATTT |
| | | GATAATACGCGAGGAACAGGTGCCTATGGAGCTTGTGCAGCGCGG |
| | | AGATATCGTCAAGGTGGTGCCCGGCGGCAAGTTCCCAGTGGATGG |
| | | AAAAGTGTTAGAGGGCAATACCATGGCCGATGAATCCCTCATCAC |
| | | TGGCGAGGCAATGCCGGTGACCAAGAAACCTGGAAGTACTGTCAT |
| | | TGCAGGCAGCATCAATGCCCATGGGAGCGTCCTGATCAAGGCCAC |
| | | CCACGTGGGCAATGATACAACTCTCGCCCAGATCGTCAAGCTAGT |
| | | GGAAGAGGCTCAGATGTCTAAAGCTCCAATCCAGCAACTGGCTGA |
| | | CCGCTTCAGCGGCTACTTTGTCCCCTTTATCATCATCATGAGCACCC |
| | | TCACCCTGGTGGTGTGGATAGTCATAGGCTTCATTGACTTTGGCGT |
| | | GGTGCAGAGATACTTCCCCAACCCTAACAAACATATCTCCCAAACC |
| | | GAGGTCATTATCAGATTCGCCTTTCAGACATCTATCACTGTGCTGT |
| | | GCATAGCCTGCCCTTGCTCTCTGGGCCTGGCCACCCCCACCGCCGT |
| | | GATGGTTGGAACCGGGGTCGCTGCACAAAATGGGATCCTGATAAA |
| | | AGGGGGCAAACCCCTGGAAATGGCACATAAGATTAAAACAGTTAT |
| | | GTTTGATAAGACAGGCACCATTACTCACGGTGTCCCTCGAGTCATG |
| | | AGAGTGCTGCTGCTGGGGGACGTGGCTACACTGCCACTGCGCAAG |
| | | GTGCTGGCTGTGGTGGGCACAGCGGAGGCCTCCTCAGAACACCCA |
| | | TTAGGCGTAGCTGTCACAAAATACTGTAAGGAGGAGCTGGGGACA |
| | | GAGACCCTCGGCTATTGTACAGACTTCCAGGCCGTGCCTGGCTGCG |
| | | GCATCGGCTGTAAGGTCAGCAATGTGGAGGGCATCCTGGCACACA |
| | | GCGAGAGACCCCTCTCTGCCCCAGCCTCCCACCTGAATGAAGCTGG |
| | | GTCTCTGCCTGCTGAGAAGGACGCCGTCCCTCAGACCTTCTCCGTG |
| | | TTGATTGGGAACAGAGAGTGGCTTAGAAGGAATGGCCTGACCATC |
| | | AGCTCTGACGTCTCGGATGCCATGACAGATCACGAGATGAAAGGC |
| | | CAGACAGCCATACTGGTTGCCATCGACGGCGTGCTCTGTGGCATGA |
| | | TTGCCATCGCTGATGCTGTTAAACAGGAAGCTGCGCTGGCAGTCCA |
| | | CACCCTCCAGTCCATGGGAGTGGACGTGGTCCTGATTACTGGAGAC |
| | | AACAGGAAAACCGCTCGGGCCATCGCTACCCAAGTGGGGATAAAT |
| | | AAGGTTTTTGCAGAAGTCTTACCCTCCCACAAAGTAGCCAAAGTGC |
| | | AGGAGCTCCAGAACAAGGGTAAAAAGGTGGCAATGGTGGGAGAT |
| | | GGTGTGAATGACAGCCCAGCTCTCGCGCAGGCTGACATGGGCGTA |
| | | GCCATAGGTACTGGAACAGATGTCGCGATTGAAGCCGCTGACGTA |
| | | GTCCTCATCCGAAACGATTTGCTGGATGTGGTGGCCAGCATCCATC |
| | | TGTCCAAGCGTACCGTGCGCCGGATCCGCATCAACCTGGTCTTGGC |
| | | CCTCATCTATAATCTCGTGGGCATCCCCATCGCTGCTGGGGTCTTC |
| | | ATGCCCATCGGCATTGTGCTGCAGCCCTGGATGGGAAGCGCTGCC |
| | | ATGGCTGCTTCTTCTGTCTCCGTTGTGCTGTCCAGCCTACAGCTGAA |
| | | GTGCTATAAAAAGCCAGACCTCGAGCGCTACGAAGCCCAGGCGCA |
| | | TGGCCATATGAAGCCCCTCACGGCTTCCCAGGTCTCGGTTCACATT |
| | | GGGATGGATGACAGATGGCGGGACTCCCCACGCGCCACACCCTGG |
| | | GACCAGGTATCCTATGTGAGCCAGGTGAGCCTCAGCTCCCTGACAT |
| | | CAGATAAACCGTCTAGGCACTCAGCTGCCGCAGATGATGACGGTG |
| | | ACAAATGGAGCCTCCTCCTGAACGGCCGCGACGAGGAGCAGTACA |
| | | TCTAAGAATTCGATATCAAGCTTATCGATAATCAACCTCTGGATTA |
| | | CAAAATTTGTGAAAGATTGACTGGTATTCTTAACTATGTTGCTCCT |
| | | TTTACGCTATGTGGATACGCTGCTTTAATGCCTTTGTATCATGCTAT |
| | | TGCTTCCCGTATGGCTTTCATTTTCTCCTCCTTGTATAAATCCTGGT |
| | | TGCTGTCTCTTTATGAGGAGTTGTGGCCCGTTGTCAGGCAACGTGG |
| | | CGTGGTGTGCACTGTGTTTGCTGACGCAACCCCCACTGGTTGGGGC |
| | | ACCTGCAGGAATAAAAGATCTTTATTTTCATTAGATCTGTGTGTTG |
| | | GTTTTTTGTGTGTCTAGAGTAGATAAGTAGCATGGCGGGTTAATCA |
| | | TTAACTACAAGGAACCCCTAGTGATGGAGTTGGCCACTCCCTCTCT |
| | | GCGCGCTCGCTCGCTCACTGAGGCCGGGCGACCAAAGGTCGCCCG |
| | | ACGCCCGGGCTTTGCCCGGGCGGCCTCAGTGAGCGAGCGAGCGCG |
| | | CAGAGAGGGAGTGGCCAA |
| 72 | A19 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTAATAATTACCAGCGCGGGCCAAAT |
| | | AAATAATCCGCGAGGGGCAGCTGGACTTTGGACTCCGCGCGCTGG |
| | | TAATTATTAACCTCGCGAATATTGATTCGAGGCCGCGATTGCCGCA |
| | | ATCGCGAGGGGCAGGTGACCTTTGCCCAGCGCGCGTTCGCCCCGC |
| | | CCCGGACGGTATCGATAAGCTTAGGAGCTTGGGCTGCAGGTCGAG |
| | | GGCACTGGGAGGATGTTGAGTAAGATGGAAAACTACTGATGACCC |
| | | TTGCAGAGACAGAGTATTAGGACATGTTTGAACAGGGGCCGGGCG |
| | | ATCAGCAGGTAGCTCTAGAGGATCCCCGTCTGTCTGCACATTTCGT |
| | | AGAGCGAGTGTTCCGATACTCTAATCTCCCTAGGCAAGGTTCATAT |
| | | TTGTGTAGGTTACTTATTCTCCTTTTGTTGACTAAGTCAATAATCAG |
| | | AATCAGCAGGTTTGGAGTCAGCTTGGCAGGGATCAGCAGCCTGGG |
| | | TTGGAAGGAGGGGGTATAAAAGCCCCTTCACCAGGAGAAGCCGTC |
| | | ACACAGATCCACAAGCTCGAGCCGAGTGGTCGTGCCTCCATAGAA |
| | | GCGGCCGCAACTCCTAAAAAACCGCCAAGATGGCGGCCCCGGAAC |
| | | AAGAACGCCAGATCACAGCAAGAGAAGGAGCAAGTAGGAAGATC |
| | | CTGAGCAAGCTCTCCCTGCCCACAAGGGCCTGGGAGCCGGCCATG |
| | | AAGAAGTCTTTTGCCTTCGATAATGTCGGTTATGAAGGCGGGCTCG |
| | | ATGGCCTGGGCCCCAGCAGCCAGGTGGCCAAGTGTTTCCTGCAGA |
| | | TCAAGGGCATGACCTGTGCCAGCTGTGTTAGCAATATCGAAAGAA |
| | | ACCTCCAAAAAGAGGCTGGAGTTCTGAGTGTTCTAGTGGCTTTAAT |
| | | GGCCGGGAAAGCCGAGATCAAGTATGATCCCGAGGTGATTCAGCC |
| | | ATTGGAGATTGCCCAGTTCATCCAGGATCTGGGCTTCGAGGCCGCC |
| | | GTGATGGAGGATTACGCCGGCTCTGATGGCAACATCGAACTGACC |
| | | ATCACCGGCATGACCTGTGCTTCCTGCGTGCATAATATCGAGAGCA |
| | | AACTGACGCGGACCAATGGAATCACCTATGCCAGCGTCGCCCTGG |
| | | CAACCTCTAAGGCTCTGGTGAAGTTTGACCCCGAAATCATTGGACC |
| | | TAGAGACATCATCAAAATTATTGAAGAGATTGGTTTCCACGCCAGC |
| | | TTAGCTCAGCGGAATCCCAACGCACACCATCTTGACCATAAGATG |
| | | GAAATCAAACAGTGGAAGAAGAGCTTCTTGTGTAGCCTGGTCTTC |
| | | GGGATACCCGTCATGGCACTGATGATTTACATGCTCATTCCGTCCA |
| | | ACGAGCCCCACCAGTCCATGGTGCTGGACCACAACATCATCCCTG |
| | | GGCTGTCTATCCTCAACCTGATCTTCTTTATTCTGTGCACATTTGTC |
| | | CAGCTCTTGGGAGGCTGGTATTTCTATGTACAGGCCTACAAATCCC |
| | | TGAGGCATCGAAGCGCCAACATGGACGTTTTAATTGTGCTGGCTAC |
| | | ATCCATTGCCTACGTGTACTCCCTGGTGATCTTAGTGGTTGCTGTG |
| | | GCTGAAAAGGCTGAGAGAAGCCCTGTCACCTTCTTCGACACCCCCC |
| | | CCATGCTCTTCGTGTTTATTGCACTGGGTCGCTGGCTGGAGCATTT |
| | | AGCCAAGTCCAAGACATCCGAGGCTCTGGCCAAGCTGATGTCTCT |
| | | ACAGGCCACGGAGGCCACCGTGGTGACTCTGGGTGAAGACAATTT |
| | | GATAATACGCGAGGAACAGGTGCCTATGGAGCTTGTGCAGCGCGG |
| | | AGATATCGTCAAGGTGGTGCCCGGCGGCAAGTTCCCAGTGGATGG |
| | | AAAAGTGTTAGAGGGCAATACCATGGCCGATGAATCCCTCATCAC |
| | | TGGCGAGGCAATGCCGGTGACCAAGAAACCTGGAAGTACTGTCAT |
| | | TGCAGGCAGCATCAATGCCCATGGGAGCGTCCTGATCAAGGCCAC |
| | | CCACGTGGGCAATGATACAACTCTCGCCCAGATCGTCAAGCTAGT |
| | | GGAAGAGGCTCAGATGTCTAAAGCTCCAATCCAGCAACTGGCTGA |
| | | CCGCTTCAGCGGCTACTTTGTCCCCTTTATCATCATCATGAGCACCC |
| | | TCACCCTGGTGGTGTGGATAGTCATAGGCTTCATTGACTTTGGCGT |
| | | GGTGCAGAGATACTTCCCCAACCCTAACAAACATATCTCCCAAACC |
| | | GAGGTCATTATCAGATTCGCCTTTCAGACATCTATCACTGTGCTGT |
| | | GCATAGCCTGCCCTTGCTCTCTGGGCCTGGCCACCCCCACCGCCGT |
| | | GATGGTTGGAACCGGGGTCGCTGCACAAAATGGGATCCTGATAAA |
| | | AGGGGGCAAACCCCTGGAAATGGCACATAAGATTAAAACAGTTAT |
| | | GTTTGATAAGACAGGCACCATTACTCACGGTGTCCCTCGAGTCATG |
| | | AGAGTGCTGCTGCTGGGGGACGTGGCTACACTGCCACTGCGCAAG |
| | | GTGCTGGCTGTGGTGGGCACAGCGGAGGCCTCCTCAGAACACCCA |
| | | TTAGGCGTAGCTGTCACAAAATACTGTAAGGAGGAGCTGGGGACA |
| | | GAGACCCTCGGCTATTGTACAGACTTCCAGGCCGTGCCTGGCTGCG |
| | | GCATCGGCTGTAAGGTCAGCAATGTGGAGGGCATCCTGGCACACA |
| | | GCGAGAGACCCCTCTCTGCCCCAGCCTCCCACCTGAATGAAGCTGG |
| | | GTCTCTGCCTGCTGAGAAGGACGCCGTCCCTCAGACCTTCTCCGTG |
| | | TTGATTGGGAACAGAGAGTGGCTTAGAAGGAATGGCCTGACCATC |
| | | AGCTCTGACGTCTCGGATGCCATGACAGATCACGAGATGAAAGGC |
| | | CAGACAGCCATACTGGTTGCCATCGACGGCGTGCTCTGTGGCATGA |
| | | TTGCCATCGCTGATGCTGTTAAACAGGAAGCTGCGCTGGCAGTCCA |
| | | CACCCTCCAGTCCATGGGAGTGGACGTGGTCCTGATTACTGGAGAC |
| | | AACAGGAAAACCGCTCGGGCCATCGCTACCCAAGTGGGGATAAAT |
| | | AAGGTTTTTGCAGAAGTCTTACCCTCCCACAAAGTAGCCAAAGTGC |
| | | AGGAGCTCCAGAACAAGGGTAAAAAGGTGGCAATGGTGGGAGAT |
| | | GGTGTGAATGACAGCCCAGCTCTCGCGCAGGCTGACATGGGCGTA |
| | | GCCATAGGTACTGGAACAGATGTCGCGATTGAAGCCGCTGACGTA |
| | | GTCCTCATCCGAAACGATTTGCTGGATGTGGTGGCCAGCATCCATC |
| | | TGTCCAAGCGTACCGTGCGCCGGATCCGCATCAACCTGGTCTTGGC |
| | | CCTCATCTATAATCTCGTGGGCATCCCCATCGCTGCTGGGGTCTTC |
| | | ATGCCCATCGGCATTGTGCTGCAGCCCTGGATGGGAAGCGCTGCC |
| | | ATGGCTGCTTCTTCTGTCTCCGTTGTGCTGTCCAGCCTACAGCTGAA |
| | | GTGCTATAAAAAGCCAGACCTCGAGCGCTACGAAGCCCAGGCGCA |
| | | TGGCCATATGAAGCCCCTCACGGCTTCCCAGGTCTCGGTTCACATT |
| | | GGGATGGATGACAGATGGCGGGACTCCCCACGCGCCACACCCTGG |
| | | GACCAGGTATCCTATGTGAGCCAGGTGAGCCTCAGCTCCCTGACAT |
| | | CAGATAAACCGTCTAGGCACTCAGCTGCCGCAGATGATGACGGTG |
| | | ACAAATGGAGCCTCCTCCTGAACGGCCGCGACGAGGAGCAGTACA |
| | | TCTAAGAATTCGATATCAAGCTTATCGATAATCAACCTCTGGATTA |
| | | CAAAATTTGTGAAAGATTGACTGGTATTCTTAACTATGTTGCTCCT |
| | | TTTACGCTATGTGGATACGCTGCTTTAATGCCTTTGTATCATGCTAT |
| | | TGCTTCCCGTATGGCTTTCATTTTCTCCTCCTTGTATAAATCCTGGT |
| | | TGCTGTCTCTTTATGAGGAGTTGTGGCCCGTTGTCAGGCAACGTGG |
| | | CGTGGTGTGCACTGTGTTTGCTGACGCAACCCCCACTGGTTGGGGC |
| | | ACCTGCAGGAATAAAAGATCTTTATTTTCATTAGATCTGTGTGTTG |
| | | GTTTTTTGTGTGTCTAGAGTAGATAAGTAGCATGGCGGGTTAATCA |
| | | TTAACTACAAGGAACCCCTAGTGATGGAGTTGGCCACTCCCTCTCT |
| | | GCGCGCTCGCTCGCTCACTGAGGCCGGGCGACCAAAGGTCGCCCG |
| | | ACGCCCGGGCTTTGCCCGGGCGGCCTCAGTGAGCGAGCGAGCGCG |
| | | CAGAGAGGGAGTGGCCAA |
| 73 | A20 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCAACTCCTAAAAAACC |
| | | GCCACCATGCCCGAACAGGAAAGACAGATTACCGCCCGCGAGGGC |
| | | GCTTCCCGCAAAATTCTGAGCAAACTGAGCCTGCCCACCCGCGCCT |
| | | GGGAACCTGCCATGAAGAAAAGCTTTGCTTTTGATAATGTCGGCTA |
| | | TGAGGGCGGCCTGGACGGCCTGGGCCCTAGCAGCCAGGTGGCTAC |
| | | CACCCTGATTGCCATTGCCGGCATGACCTGCGCCAGCTGCGTGCAT |
| | | AGCATTGAGGGCATGATTAGCCAGCTGGAGGGCGTGCAGCAGATT |
| | | TCCGTGAGCCTGGCTGAGGGCACCGCTACCGTGCTGTATAATCCCT |
| | | CCGTGATTAGCCCCGAAGAACTGCGCGCTGCCATTGAAGATATGG |
| | | GCTTTGAAGCCTCCGTGGTGTCCGAAAGCTGCAGCACCAATCCCCT |
| | | GGGCAATCATTCCGCCGGCAATAGCATGGTGCAGACCACCGACGG |
| | | CACCCCCACCTCCGTGCAGGAAGTGGCTCCCCATACCGGCCGCCTG |
| | | CCCGCCAATCATGCCCCTGATATTCTGGCTAAAAGCCCCCAGAGCA |
| | | CCCGCGCCGTGGCTCCTCAGAAATGCTTTCTGCAGATTAAGGGCAT |
| | | GACCTGCGCTAGCTGCGTGAGCAATATTGAACGCAATCTGCAGAA |
| | | AGAAGCCGGCGTGCTGTCCGTGCTGGTGGCCCTGATGGCCGGCAA |
| | | AGCTGAAATCAAATATGATCCCGAAGTGATTCAGCCCCTGGAAAT |
| | | TGCCCAGTTCATTCAGGATCTGGGCTTTGAGGCTGCCGTGATGGAA |
| | | GATTATGCCGGCTCCGACGGCAATATTGAACTGACCATTACCGGCA |
| | | TGACCTGCGCCAGCTGCGTGCATAATATTGAAAGCAAACTGACCC |
| | | GCACCAACGGCATTACCTATGCTTCCGTGGCCCTGGCTACCAGCAA |
| | | AGCCCTGGTGAAATTTGATCCTGAAATTATCGGCCCCCGCGATATT |
| | | ATCAAAATTATTGAAGAAATCGGCTTTCACGCCAGCCTGGCTCAGC |
| | | GCAATCCCAATGCCCATCATCTGGATCATAAGATGGAAATCAAAC |
| | | AGTGGAAGAAAAGCTTTCTGTGCAGCCTGGTGTTCGGCATTCCTGT |
| | | GATGGCCCTGATGATCTATATGCTGATTCCCAGCAATGAACCCCAT |
| | | CAGAGCATGGTGCTGGATCATAATATTATTCCCGGCCTGAGCATTC |
| | | TGAATCTGATTTTCTTTATCCTGTGCACCTTTGTGCAGCTGCTGGGC |
| | | GGCTGGTATTTCTATGTGCAGGCTTATAAAAGCCTGCGCCACCGCT |
| | | CCGCCAATATGGATGTGCTGATTGTGCTGGCCACCAGCATTGCTTA |
| | | TGTCTATAGCCTGGTCATTCTGGTGGTGGCCGTGGCCGAGAAAGCT |
| | | GAACGCAGCCCCGTGACCTTCTTTGATACCCCTCCCATGCTGTTCG |
| | | TGTTTATCGCTCTGGGCCGCTGGCTGGAACATCTGGCCAAAAGCAA |
| | | AACCTCCGAAGCCCTGGCTAAACTGATGAGCCTGCAGGCCACCGA |
| | | AGCCACCGTGGTGACCCTGGGCGAAGATAATCTGATTATCCGCGA |
| | | AGAACAGGTGCCTATGGAACTGGTGCAGCGCGGCGATATTGTGAA |
| | | AGTGGTGCCCGGCGGCAAATTTCCCGTGGACGGCAAAGTGCTGGA |
| | | GGGCAATACCATGGCTGATGAATCCCTGATTACCGGCGAAGCTAT |
| | | GCCTGTGACCAAGAAACCCGGCAGCACCGTGATTGCCGGCAGCAT |
| | | TAATGCTCACGGCTCCGTGCTGATCAAGGCTACCCATGTCGGCAAT |
| | | GATACCACCCTGGCCCAGATTGTGAAACTGGTGGAAGAAGCCCAG |
| | | ATGAGCAAAGCCCCTATTCAGCAGCTGGCTGACCGCTTTTCCGGCT |
| | | ATTTTGTGCCCTTTATCATTATTATGAGCACCCTGACCCTGGTGGTG |
| | | TGGATTGTGATCGGCTTCATTGATTTCGGCGTGGTGCAGAGATATT |
| | | TTCCTAATCCCAACAAACATATTAGCCAGACCGAAGTGATTATCCG |
| | | CTTTGCTTTTCAGACCAGCATTACCGTGCTGTGCATTGCTTGCCCCT |
| | | GCAGCCTGGGCCTGGCTACCCCCACCGCCGTCATGGTCGGCACCG |
| | | GCGTGGCTGCCCAGAACGGCATTCTGATTAAGGGCGGCAAACCTC |
| | | TGGAAATGGCCCACAAAATCAAAACCGTGATGTTTGATAAAACCG |
| | | GCACCATTACCCACGGCGTGCCCCGCGTGATGCGCGTGCTGCTGCT |
| | | GGGCGATGTGGCTACCCTGCCCCTGCGCAAAGTGCTGGCCGTGGTC |
| | | GGCACCGCCGAAGCTAGCTCCGAACATCCCCTGGGCGTGGCCGTG |
| | | ACCAAATATTGCAAAGAAGAACTGGGCACCGAAACCCTGGGCTAT |
| | | TGCACCGATTTTCAGGCCGTCCCTGGCTGCGGCATCGGCTGCAAAG |
| | | TGAGCAATGTGGAGGGCATTCTGGCCCATTCCGAAAGACCCCTGTC |
| | | CGCCCCTGCTAGCCATCTGAATGAAGCCGGCAGCCTGCCTGCTGAG |
| | | AAAGATGCCGTGCCCCAGACCTTTTCCGTGCTGATCGGCAACCGCG |
| | | AATGGCTGCGCCGCAACGGCCTGACCATTAGCTCCGATGTCTCCGA |
| | | TGCCATGACCGATCATGAAATGAAAGGCCAGACCGCCATTCTGGT |
| | | GGCCATTGACGGCGTGCTGTGCGGCATGATTGCCATTGCCGATGCC |
| | | GTGAAACAGGAGGCTGCCCTGGCTGTGCATACCCTGCAGAGCATG |
| | | GGCGTGGATGTGGTGCTGATTACCGGCGATAACCGCAAAACCGCT |
| | | CGCGCCATTGCCACCCAGGTCGGCATTAACAAAGTGTTTGCTGAAG |
| | | TGCTGCCCAGCCATAAAGTGGCTAAAGTGCAGGAACTGCAGAACA |
| | | AAGGCAAGAAAGTGGCTATGGTCGGCGACGGCGTGAATGATAGCC |
| | | CTGCCCTGGCCCAGGCCGATATGGGCGTGGCTATCGGCACCGGCA |
| | | CCGATGTGGCTATTGAGGCTGCCGATGTGGTGCTGATCCGCAATGA |
| | | TCTGCTGGATGTGGTGGCTAGCATTCATCTGAGCAAACGCACCGTC |
| | | CGCCGCATCCGCATTAATCTGGTGCTGGCCCTGATCTATAATCTGG |
| | | TCGGCATTCCCATCGCCGCCGGCGTGTTTATGCCCATCGGCATTGT |
| | | GCTGCAGCCCTGGATGGGCAGCGCCGCTATGGCCGCTAGCTCCGT |
| | | GTCCGTGGTGCTGAGCAGCCTGCAGCTGAAATGCTATAAGAAACC |
| | | CGATCTGGAAAGATATGAAGCTCAGGCTCACGGCCATATGAAACC |
| | | CCTGACCGCCAGCCAGGTGTCCGTGCATATCGGCATGGATGACCG |
| | | CTGGCGCGATAGCCCCCGCGCCACCCCTTGGGATCAGGTGAGCTAT |
| | | GTGAGCCAGGTGTCCCTGAGCTCCCTGACCTCCGATAAACCCTCCC |
| | | GCCATTCCGCCGCTGCCGATGATGACGGCGATAAGTGGAGCCTGC |
| | | TGCTGAACGGCCGCGATGAAGAACAGTATATCTAAGTCCATAAAG |
| | | TAGGAAACACTACATCATCCATAAAGTAGGAAACACTACATCATC |
| | | CATAAAGTAGGAAACACTACATCATCCATAAAGTAGGAAACACTA |
| | | CAGAATTCGATATCAAGCTTATCGATAATCAACCTCTGGATTACAA |
| | | AATTTGTGAAAGATTGACTGGTATTCTTAACTATGTTGCTCCTTTTA |
| | | CGCTATGTGGATACGCTGCTTTAATGCCTTTGTATCATGCTATTGCT |
| | | TCCCGTATGGCTTTCATTTTCTCCTCCTTGTATAAATCCTGGTTGCT |
| | | GTCTCTTTATGAGGAGTTGTGGCCCGTTGTCAGGCAACGTGGCGTG |
| | | GTGTGCACTGTGTTTGCTGACGCAACCCCCACTGGTTGGGGCACCT |
| | | GCAGGAATAAAAGATCTTTATTTTCATTAGATCTGTGTGTTGGTTTT |
| | | TTGTGTGTCTAGAGTAGATAAGTAGCATGGCGGGTTAATCATTAAC |
| | | TACAAGGAACCCCTAGTGATGGAGTTGGCCACTCCCTCTCTGCGCG |
| | | CTCGCTCGCTCACTGAGGCCGGGCGACCAAAGGTCGCCCGACGCC |
| | | CGGGCTTTGCCCGGGCGGCCTCAGTGAGCGAGCGAGCGCGCAGAG |
| | | AGGGAGTGGCCAA |
| 74 | A21 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCAACTCCTAAAAAACC |
| | | GCCACCATGCCCGAACAGGAAAGACAGATTACCGCCCGCGAGGGC |
| | | GCTTCCCGCAAAATTCTGAGCAAACTGAGCCTGCCCACCCGCGCCT |
| | | GGGAACCTGCCATGAAGAAAAGCTTTGCTTTTGATAATGTCGGCTA |
| | | TGAGGGCGGCCTGGACGGCCTGGGCCCTAGCAGCCAGGTGGCTAC |
| | | CACCCTGATTGCCATTGCCGGCATGACCTGCGCCAGCTGCGTGCAT |
| | | AGCATTGAGGGCATGATTAGCCAGCTGGAGGGCGTGCAGCAGATT |
| | | TCCGTGAGCCTGGCTGAGGGCACCGCTACCGTGCTGTATAATCCCT |
| | | CCGTGATTAGCCCCGAAGAACTGCGCGCTGCCATTGAAGATATGG |
| | | GCTTTGAAGCCTCCGTGGTGTCCGAAAGCTGCAGCACCAATCCCCT |
| | | GGGCAATCATTCCGCCGGCAATAGCATGGTGCAGACCACCGACGG |
| | | CACCCCCACCTCCGTGCAGGAAGTGGCTCCCCATACCGGCCGCCTG |
| | | CCCGCCAATCATGCCCCTGATATTCTGGCTAAAAGCCCCCAGAGCA |
| | | CCCGCGCCGTGGCTCCTCAGAAATGCTTTCTGCAGATTAAGGGCAT |
| | | GACCTGCGCTAGCTGCGTGAGCAATATTGAACGCAATCTGCAGAA |
| | | AGAAGCCGGCGTGCTGTCCGTGCTGGTGGCCCTGATGGCCGGCAA |
| | | AGCTGAAATCAAATATGATCCCGAAGTGATTCAGCCCCTGGAAAT |
| | | TGCCCAGTTCATTCAGGATCTGGGCTTTGAGGCTGCCGTGATGGAA |
| | | GATTATGCCGGCTCCGACGGCAATATTGAACTGACCATTACCGGCA |
| | | TGACCTGCGCCAGCTGCGTGCATAATATTGAAAGCAAACTGACCC |
| | | GCACCAACGGCATTACCTATGCTTCCGTGGCCCTGGCTACCAGCAA |
| | | AGCCCTGGTGAAATTTGATCCTGAAATTATCGGCCCCCGCGATATT |
| | | ATCAAAATTATTGAAGAAATCGGCTTTCACGCCAGCCTGGCTCAGC |
| | | GCAATCCCAATGCCCATCATCTGGATCATAAGATGGAAATCAAAC |
| | | AGTGGAAGAAAAGCTTTCTGTGCAGCCTGGTGTTCGGCATTCCTGT |
| | | GATGGCCCTGATGATCTATATGCTGATTCCCAGCAATGAACCCCAT |
| | | CAGAGCATGGTGCTGGATCATAATATTATTCCCGGCCTGAGCATTC |
| | | TGAATCTGATTTTCTTTATCCTGTGCACCTTTGTGCAGCTGCTGGGC |
| | | GGCTGGTATTTCTATGTGCAGGCTTATAAAAGCCTGCGCCACCGCT |
| | | CCGCCAATATGGATGTGCTGATTGTGCTGGCCACCAGCATTGCTTA |
| | | TGTCTATAGCCTGGTCATTCTGGTGGTGGCCGTGGCCGAGAAAGCT |
| | | GAACGCAGCCCCGTGACCTTCTTTGATACCCCTCCCATGCTGTTCG |
| | | TGTTTATCGCTCTGGGCCGCTGGCTGGAACATCTGGCCAAAAGCAA |
| | | AACCTCCGAAGCCCTGGCTAAACTGATGAGCCTGCAGGCCACCGA |
| | | AGCCACCGTGGTGACCCTGGGCGAAGATAATCTGATTATCCGCGA |
| | | AGAACAGGTGCCTATGGAACTGGTGCAGCGCGGCGATATTGTGAA |
| | | AGTGGTGCCCGGCGGCAAATTTCCCGTGGACGGCAAAGTGCTGGA |
| | | GGGCAATACCATGGCTGATGAATCCCTGATTACCGGCGAAGCTAT |
| | | GCCTGTGACCAAGAAACCCGGCAGCACCGTGATTGCCGGCAGCAT |
| | | TAATGCTCACGGCTCCGTGCTGATCAAGGCTACCCATGTCGGCAAT |
| | | GATACCACCCTGGCCCAGATTGTGAAACTGGTGGAAGAAGCCCAG |
| | | ATGAGCAAAGCCCCTATTCAGCAGCTGGCTGACCGCTTTTCCGGCT |
| | | ATTTTGTGCCCTTTATCATTATTATGAGCACCCTGACCCTGGTGGTG |
| | | TGGATTGTGATCGGCTTCATTGATTTCGGCGTGGTGCAGAGATATT |
| | | TTCCTAATCCCAACAAACATATTAGCCAGACCGAAGTGATTATCCG |
| | | CTTTGCTTTTCAGACCAGCATTACCGTGCTGTGCATTGCTTGCCCCT |
| | | GCAGCCTGGGCCTGGCTACCCCCACCGCCGTCATGGTCGGCACCG |
| | | GCGTGGCTGCCCAGAACGGCATTCTGATTAAGGGCGGCAAACCTC |
| | | TGGAAATGGCCCACAAAATCAAAACCGTGATGTTTGATAAAACCG |
| | | GCACCATTACCCACGGCGTGCCCCGCGTGATGCGCGTGCTGCTGCT |
| | | GGGCGATGTGGCTACCCTGCCCCTGCGCAAAGTGCTGGCCGTGGTC |
| | | GGCACCGCCGAAGCTAGCTCCGAACATCCCCTGGGCGTGGCCGTG |
| | | ACCAAATATTGCAAAGAAGAACTGGGCACCGAAACCCTGGGCTAT |
| | | TGCACCGATTTTCAGGCCGTCCCTGGCTGCGGCATCGGCTGCAAAG |
| | | TGAGCAATGTGGAGGGCATTCTGGCCCATTCCGAAAGACCCCTGTC |
| | | CGCCCCTGCTAGCCATCTGAATGAAGCCGGCAGCCTGCCTGCTGAG |
| | | AAAGATGCCGTGCCCCAGACCTTTTCCGTGCTGATCGGCAACCGCG |
| | | AATGGCTGCGCCGCAACGGCCTGACCATTAGCTCCGATGTCTCCGA |
| | | TGCCATGACCGATCATGAAATGAAAGGCCAGACCGCCATTCTGGT |
| | | GGCCATTGACGGCGTGCTGTGCGGCATGATTGCCATTGCCGATGCC |
| | | GTGAAACAGGAGGCTGCCCTGGCTGTGCATACCCTGCAGAGCATG |
| | | GGCGTGGATGTGGTGCTGATTACCGGCGATAACCGCAAAACCGCT |
| | | CGCGCCATTGCCACCCAGGTCGGCATTAACAAAGTGTTTGCTGAAG |
| | | TGCTGCCCAGCCATAAAGTGGCTAAAGTGCAGGAACTGCAGAACA |
| | | AAGGCAAGAAAGTGGCTATGGTCGGCGACGGCGTGAATGATAGCC |
| | | CTGCCCTGGCCCAGGCCGATATGGGCGTGGCTATCGGCACCGGCA |
| | | CCGATGTGGCTATTGAGGCTGCCGATGTGGTGCTGATCCGCAATGA |
| | | TCTGCTGGATGTGGTGGCTAGCATTCATCTGAGCAAACGCACCGTC |
| | | CGCCGCATCCGCATTAATCTGGTGCTGGCCCTGATCTATAATCTGG |
| | | TCGGCATTCCCATCGCCGCCGGCGTGTTTATGCCCATCGGCATTGT |
| | | GCTGCAGCCCTGGATGGGCAGCGCCGCTATGGCCGCTAGCTCCGT |
| | | GTCCGTGGTGCTGAGCAGCCTGCAGCTGAAATGCTATAAGAAACC |
| | | CGATCTGGAAAGATATGAAGCTCAGGCTCACGGCCATATGAAACC |
| | | CCTGACCGCCAGCCAGGTGTCCGTGCATATCGGCATGGATGACCG |
| | | CTGGCGCGATAGCCCCCGCGCCACCCCTTGGGATCAGGTGAGCTAT |
| | | GTGAGCCAGGTGTCCCTGAGCTCCCTGACCTCCGATAAACCCTCCC |
| | | GCCATTCCGCCGCTGCCGATGATGACGGCGATAAGTGGAGCCTGC |
| | | TGCTGAACGGCCGCGATGAAGAACAGTATATCTAAGTCCATAAAG |
| | | TAGGAAACACTACATCATCCATAAAGTAGGAAACACTACATCATC |
| | | CATAAAGTAGGAAACACTACATCATCCATAAAGTAGGAAACACTA |
| | | CAGAATTCGATATCAAGCTTATCGATAATCAACCTCTGGATTACAA |
| | | AATTTGTGAAAGATTGACTGGTATTCTTAACTATGTTGCTCCTTTTA |
| | | CGCTATGTGGATACGCTGCTTTAATGCCTTTGTATCATGCTATTGCT |
| | | TCCCGTATGGCTTTCATTTTCTCCTCCTTGTATAAATCCTGGTTAGT |
| | | TCTTGCCACGGCGGAACTCATCGCCGCCTGCCTTGCCCGCTGCTGG |
| | | ACAGGGGCTCGGCTGTTGGGCACTGACAATTCCGTGGTGTTCACCT |
| | | GCAGGAATAAAAGATCTTTATTTTCATTAGATCTGTGTGTTGGTTTT |
| | | TTGTGTGTCTAGAGTAGATAAGTAGCATGGCGGGTTAATCATTAAC |
| | | TACAAGGAACCCCTAGTGATGGAGTTGGCCACTCCCTCTCTGCGCG |
| | | CTCGCTCGCTCACTGAGGCCGGGCGACCAAAGGTCGCCCGACGCC |
| | | CGGGCTTTGCCCGGGCGGCCTCAGTGAGCGAGCGAGCGCGCAGAG |
| | | AGGGAGTGGCCAA |
| 75 | A22 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCAACTCCTAAAAAACC |
| | | GCCACCATGCCCGAACAGGAAAGACAGATTACCGCCCGCGAGGGC |
| | | GCTTCCCGCAAAATTCTGAGCAAACTGAGCCTGCCCACCCGCGCCT |
| | | GGGAACCTGCCATGAAGAAAAGCTTTGCTTTTGATAATGTCGGCTA |
| | | TGAGGGCGGCCTGGACGGCCTGGGCCCTAGCAGCCAGGTGGCTAC |
| | | CACCCTGATTGCCATTGCCGGCATGACCTGCGCCAGCTGCGTGCAT |
| | | AGCATTGAGGGCATGATTAGCCAGCTGGAGGGCGTGCAGCAGATT |
| | | TCCGTGAGCCTGGCTGAGGGCACCGCTACCGTGCTGTATAATCCCT |
| | | CCGTGATTAGCCCCGAAGAACTGCGCGCTGCCATTGAAGATATGG |
| | | GCTTTGAAGCCTCCGTGGTGTCCGAAAGCTGCAGCACCAATCCCCT |
| | | GGGCAATCATTCCGCCGGCAATAGCATGGTGCAGACCACCGACGG |
| | | CACCCCCACCTCCGTGCAGGAAGTGGCTCCCCATACCGGCCGCCTG |
| | | CCCGCCAATCATGCCCCTGATATTCTGGCTAAAAGCCCCCAGAGCA |
| | | CCCGCGCCGTGGCTCCTCAGAAATGCTTTCTGCAGATTAAGGGCAT |
| | | GACCTGCGCTAGCTGCGTGAGCAATATTGAACGCAATCTGCAGAA |
| | | AGAAGCCGGCGTGCTGTCCGTGCTGGTGGCCCTGATGGCCGGCAA |
| | | AGCTGAAATCAAATATGATCCCGAAGTGATTCAGCCCCTGGAAAT |
| | | TGCCCAGTTCATTCAGGATCTGGGCTTTGAGGCTGCCGTGATGGAA |
| | | GATTATGCCGGCTCCGACGGCAATATTGAACTGACCATTACCGGCA |
| | | TGACCTGCGCCAGCTGCGTGCATAATATTGAAAGCAAACTGACCC |
| | | GCACCAACGGCATTACCTATGCTTCCGTGGCCCTGGCTACCAGCAA |
| | | AGCCCTGGTGAAATTTGATCCTGAAATTATCGGCCCCCGCGATATT |
| | | ATCAAAATTATTGAAGAAATCGGCTTTCACGCCAGCCTGGCTCAGC |
| | | GCAATCCCAATGCCCATCATCTGGATCATAAGATGGAAATCAAAC |
| | | AGTGGAAGAAAAGCTTTCTGTGCAGCCTGGTGTTCGGCATTCCTGT |
| | | GATGGCCCTGATGATCTATATGCTGATTCCCAGCAATGAACCCCAT |
| | | CAGAGCATGGTGCTGGATCATAATATTATTCCCGGCCTGAGCATTC |
| | | TGAATCTGATTTTCTTTATCCTGTGCACCTTTGTGCAGCTGCTGGGC |
| | | GGCTGGTATTTCTATGTGCAGGCTTATAAAAGCCTGCGCCACCGCT |
| | | CCGCCAATATGGATGTGCTGATTGTGCTGGCCACCAGCATTGCTTA |
| | | TGTCTATAGCCTGGTCATTCTGGTGGTGGCCGTGGCCGAGAAAGCT |
| | | GAACGCAGCCCCGTGACCTTCTTTGATACCCCTCCCATGCTGTTCG |
| | | TGTTTATCGCTCTGGGCCGCTGGCTGGAACATCTGGCCAAAAGCAA |
| | | AACCTCCGAAGCCCTGGCTAAACTGATGAGCCTGCAGGCCACCGA |
| | | AGCCACCGTGGTGACCCTGGGCGAAGATAATCTGATTATCCGCGA |
| | | AGAACAGGTGCCTATGGAACTGGTGCAGCGCGGCGATATTGTGAA |
| | | AGTGGTGCCCGGCGGCAAATTTCCCGTGGACGGCAAAGTGCTGGA |
| | | GGGCAATACCATGGCTGATGAATCCCTGATTACCGGCGAAGCTAT |
| | | GCCTGTGACCAAGAAACCCGGCAGCACCGTGATTGCCGGCAGCAT |
| | | TAATGCTCACGGCTCCGTGCTGATCAAGGCTACCCATGTCGGCAAT |
| | | GATACCACCCTGGCCCAGATTGTGAAACTGGTGGAAGAAGCCCAG |
| | | ATGAGCAAAGCCCCTATTCAGCAGCTGGCTGACCGCTTTTCCGGCT |
| | | ATTTTGTGCCCTTTATCATTATTATGAGCACCCTGACCCTGGTGGTG |
| | | TGGATTGTGATCGGCTTCATTGATTTCGGCGTGGTGCAGAGATATT |
| | | TTCCTAATCCCAACAAACATATTAGCCAGACCGAAGTGATTATCCG |
| | | CTTTGCTTTTCAGACCAGCATTACCGTGCTGTGCATTGCTTGCCCCT |
| | | GCAGCCTGGGCCTGGCTACCCCCACCGCCGTCATGGTCGGCACCG |
| | | GCGTGGCTGCCCAGAACGGCATTCTGATTAAGGGCGGCAAACCTC |
| | | TGGAAATGGCCCACAAAATCAAAACCGTGATGTTTGATAAAACCG |
| | | GCACCATTACCCACGGCGTGCCCCGCGTGATGCGCGTGCTGCTGCT |
| | | GGGCGATGTGGCTACCCTGCCCCTGCGCAAAGTGCTGGCCGTGGTC |
| | | GGCACCGCCGAAGCTAGCTCCGAACATCCCCTGGGCGTGGCCGTG |
| | | ACCAAATATTGCAAAGAAGAACTGGGCACCGAAACCCTGGGCTAT |
| | | TGCACCGATTTTCAGGCCGTCCCTGGCTGCGGCATCGGCTGCAAAG |
| | | TGAGCAATGTGGAGGGCATTCTGGCCCATTCCGAAAGACCCCTGTC |
| | | CGCCCCTGCTAGCCATCTGAATGAAGCCGGCAGCCTGCCTGCTGAG |
| | | AAAGATGCCGTGCCCCAGACCTTTTCCGTGCTGATCGGCAACCGCG |
| | | AATGGCTGCGCCGCAACGGCCTGACCATTAGCTCCGATGTCTCCGA |
| | | TGCCATGACCGATCATGAAATGAAAGGCCAGACCGCCATTCTGGT |
| | | GGCCATTGACGGCGTGCTGTGCGGCATGATTGCCATTGCCGATGCC |
| | | GTGAAACAGGAGGCTGCCCTGGCTGTGCATACCCTGCAGAGCATG |
| | | GGCGTGGATGTGGTGCTGATTACCGGCGATAACCGCAAAACCGCT |
| | | CGCGCCATTGCCACCCAGGTCGGCATTAACAAAGTGTTTGCTGAAG |
| | | TGCTGCCCAGCCATAAAGTGGCTAAAGTGCAGGAACTGCAGAACA |
| | | AAGGCAAGAAAGTGGCTATGGTCGGCGACGGCGTGAATGATAGCC |
| | | CTGCCCTGGCCCAGGCCGATATGGGCGTGGCTATCGGCACCGGCA |
| | | CCGATGTGGCTATTGAGGCTGCCGATGTGGTGCTGATCCGCAATGA |
| | | TCTGCTGGATGTGGTGGCTAGCATTCATCTGAGCAAACGCACCGTC |
| | | CGCCGCATCCGCATTAATCTGGTGCTGGCCCTGATCTATAATCTGG |
| | | TCGGCATTCCCATCGCCGCCGGCGTGTTTATGCCCATCGGCATTGT |
| | | GCTGCAGCCCTGGATGGGCAGCGCCGCTATGGCCGCTAGCTCCGT |
| | | GTCCGTGGTGCTGAGCAGCCTGCAGCTGAAATGCTATAAGAAACC |
| | | CGATCTGGAAAGATATGAAGCTCAGGCTCACGGCCATATGAAACC |
| | | CCTGACCGCCAGCCAGGTGTCCGTGCATATCGGCATGGATGACCG |
| | | CTGGCGCGATAGCCCCCGCGCCACCCCTTGGGATCAGGTGAGCTAT |
| | | GTGAGCCAGGTGTCCCTGAGCTCCCTGACCTCCGATAAACCCTCCC |
| | | GCCATTCCGCCGCTGCCGATGATGACGGCGATAAGTGGAGCCTGC |
| | | TGCTGAACGGCCGCGATGAAGAACAGTATATCTAAGTCCATAAAG |
| | | TAGGAAACACTACATCATCCATAAAGTAGGAAACACTACATCATC |
| | | CATAAAGTAGGAAACACTACATCATCCATAAAGTAGGAAACACTA |
| | | CAGAATTCGATATCAAGCTTATCGATCCTGCAGGAATAAAAGATCT |
| | | TTATTTTCATTAGATCTGTGTGTTGGTTTTTTGTGTGTCTAGAGTAG |
| | | ATAAGTAGCATGGCGGGTTAATCATTAACTACAAGGAACCCCTAG |
| | | TGATGGAGTTGGCCACTCCCTCTCTGCGCGCTCGCTCGCTCACTGA |
| | | GGCCGGGCGACCAAAGGTCGCCCGACGCCCGGGCTTTGCCCGGGC |
| | | GGCCTCAGTGAGCGAGCGAGCGCGCAGAGAGGGAGTGGCCAA |
| 76 | A23 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCAACTCCTAAAAAACC |
| | | GCCACCATGCCCGAACAGGAAAGACAGATTACCGCCCGCGAGGGC |
| | | GCTTCCCGCAAAATTCTGAGCAAACTGAGCCTGCCCACCCGCGCCT |
| | | GGGAACCTGCCATGAAGAAAAGCTTTGCTTTTGATAATGTCGGCTA |
| | | TGAGGGCGGCCTGGACGGCCTGGGCCCTAGCAGCCAGGTGGCTAC |
| | | CACCCTGATTGCCATTGCCGGCATGACCTGCGCCAGCTGCGTGCAT |
| | | AGCATTGAGGGCATGATTAGCCAGCTGGAGGGCGTGCAGCAGATT |
| | | TCCGTGAGCCTGGCTGAGGGCACCGCTACCGTGCTGTATAATCCCT |
| | | CCGTGATTAGCCCCGAAGAACTGCGCGCTGCCATTGAAGATATGG |
| | | GCTTTGAAGCCTCCGTGGTGTCCGAAAGCTGCAGCACCAATCCCCT |
| | | GGGCAATCATTCCGCCGGCAATAGCATGGTGCAGACCACCGACGG |
| | | CACCCCCACCTCCGTGCAGGAAGTGGCTCCCCATACCGGCCGCCTG |
| | | CCCGCCAATCATGCCCCTGATATTCTGGCTAAAAGCCCCCAGAGCA |
| | | CCCGCGCCGTGGCTCCTCAGAAATGCTTTCTGCAGATTAAGGGCAT |
| | | GACCTGCGCTAGCTGCGTGAGCAATATTGAACGCAATCTGCAGAA |
| | | AGAAGCCGGCGTGCTGTCCGTGCTGGTGGCCCTGATGGCCGGCAA |
| | | AGCTGAAATCAAATATGATCCCGAAGTGATTCAGCCCCTGGAAAT |
| | | TGCCCAGTTCATTCAGGATCTGGGCTTTGAGGCTGCCGTGATGGAA |
| | | GATTATGCCGGCTCCGACGGCAATATTGAACTGACCATTACCGGCA |
| | | TGACCTGCGCCAGCTGCGTGCATAATATTGAAAGCAAACTGACCC |
| | | GCACCAACGGCATTACCTATGCTTCCGTGGCCCTGGCTACCAGCAA |
| | | AGCCCTGGTGAAATTTGATCCTGAAATTATCGGCCCCCGCGATATT |
| | | ATCAAAATTATTGAAGAAATCGGCTTTCACGCCAGCCTGGCTCAGC |
| | | GCAATCCCAATGCCCATCATCTGGATCATAAGATGGAAATCAAAC |
| | | AGTGGAAGAAAAGCTTTCTGTGCAGCCTGGTGTTCGGCATTCCTGT |
| | | GATGGCCCTGATGATCTATATGCTGATTCCCAGCAATGAACCCCAT |
| | | CAGAGCATGGTGCTGGATCATAATATTATTCCCGGCCTGAGCATTC |
| | | TGAATCTGATTTTCTTTATCCTGTGCACCTTTGTGCAGCTGCTGGGC |
| | | GGCTGGTATTTCTATGTGCAGGCTTATAAAAGCCTGCGCCACCGCT |
| | | CCGCCAATATGGATGTGCTGATTGTGCTGGCCACCAGCATTGCTTA |
| | | TGTCTATAGCCTGGTCATTCTGGTGGTGGCCGTGGCCGAGAAAGCT |
| | | GAACGCAGCCCCGTGACCTTCTTTGATACCCCTCCCATGCTGTTCG |
| | | TGTTTATCGCTCTGGGCCGCTGGCTGGAACATCTGGCCAAAAGCAA |
| | | AACCTCCGAAGCCCTGGCTAAACTGATGAGCCTGCAGGCCACCGA |
| | | AGCCACCGTGGTGACCCTGGGCGAAGATAATCTGATTATCCGCGA |
| | | AGAACAGGTGCCTATGGAACTGGTGCAGCGCGGCGATATTGTGAA |
| | | AGTGGTGCCCGGCGGCAAATTTCCCGTGGACGGCAAAGTGCTGGA |
| | | GGGCAATACCATGGCTGATGAATCCCTGATTACCGGCGAAGCTAT |
| | | GCCTGTGACCAAGAAACCCGGCAGCACCGTGATTGCCGGCAGCAT |
| | | TAATGCTCACGGCTCCGTGCTGATCAAGGCTACCCATGTCGGCAAT |
| | | GATACCACCCTGGCCCAGATTGTGAAACTGGTGGAAGAAGCCCAG |
| | | ATGAGCAAAGCCCCTATTCAGCAGCTGGCTGACCGCTTTTCCGGCT |
| | | ATTTTGTGCCCTTTATCATTATTATGAGCACCCTGACCCTGGTGGTG |
| | | TGGATTGTGATCGGCTTCATTGATTTCGGCGTGGTGCAGAGATATT |
| | | TTCCTAATCCCAACAAACATATTAGCCAGACCGAAGTGATTATCCG |
| | | CTTTGCTTTTCAGACCAGCATTACCGTGCTGTGCATTGCTTGCCCCT |
| | | GCAGCCTGGGCCTGGCTACCCCCACCGCCGTCATGGTCGGCACCG |
| | | GCGTGGCTGCCCAGAACGGCATTCTGATTAAGGGCGGCAAACCTC |
| | | TGGAAATGGCCCACAAAATCAAAACCGTGATGTTTGATAAAACCG |
| | | GCACCATTACCCACGGCGTGCCCCGCGTGATGCGCGTGCTGCTGCT |
| | | GGGCGATGTGGCTACCCTGCCCCTGCGCAAAGTGCTGGCCGTGGTC |
| | | GGCACCGCCGAAGCTAGCTCCGAACATCCCCTGGGCGTGGCCGTG |
| | | ACCAAATATTGCAAAGAAGAACTGGGCACCGAAACCCTGGGCTAT |
| | | TGCACCGATTTTCAGGCCGTCCCTGGCTGCGGCATCGGCTGCAAAG |
| | | TGAGCAATGTGGAGGGCATTCTGGCCCATTCCGAAAGACCCCTGTC |
| | | CGCCCCTGCTAGCCATCTGAATGAAGCCGGCAGCCTGCCTGCTGAG |
| | | AAAGATGCCGTGCCCCAGACCTTTTCCGTGCTGATCGGCAACCGCG |
| | | AATGGCTGCGCCGCAACGGCCTGACCATTAGCTCCGATGTCTCCGA |
| | | TGCCATGACCGATCATGAAATGAAAGGCCAGACCGCCATTCTGGT |
| | | GGCCATTGACGGCGTGCTGTGCGGCATGATTGCCATTGCCGATGCC |
| | | GTGAAACAGGAGGCTGCCCTGGCTGTGCATACCCTGCAGAGCATG |
| | | GGCGTGGATGTGGTGCTGATTACCGGCGATAACCGCAAAACCGCT |
| | | CGCGCCATTGCCACCCAGGTCGGCATTAACAAAGTGTTTGCTGAAG |
| | | TGCTGCCCAGCCATAAAGTGGCTAAAGTGCAGGAACTGCAGAACA |
| | | AAGGCAAGAAAGTGGCTATGGTCGGCGACGGCGTGAATGATAGCC |
| | | CTGCCCTGGCCCAGGCCGATATGGGCGTGGCTATCGGCACCGGCA |
| | | CCGATGTGGCTATTGAGGCTGCCGATGTGGTGCTGATCCGCAATGA |
| | | TCTGCTGGATGTGGTGGCTAGCATTCATCTGAGCAAACGCACCGTC |
| | | CGCCGCATCCGCATTAATCTGGTGCTGGCCCTGATCTATAATCTGG |
| | | TCGGCATTCCCATCGCCGCCGGCGTGTTTATGCCCATCGGCATTGT |
| | | GCTGCAGCCCTGGATGGGCAGCGCCGCTATGGCCGCTAGCTCCGT |
| | | GTCCGTGGTGCTGAGCAGCCTGCAGCTGAAATGCTATAAGAAACC |
| | | CGATCTGGAAAGATATGAAGCTCAGGCTCACGGCCATATGAAACC |
| | | CCTGACCGCCAGCCAGGTGTCCGTGCATATCGGCATGGATGACCG |
| | | CTGGCGCGATAGCCCCCGCGCCACCCCTTGGGATCAGGTGAGCTAT |
| | | GTGAGCCAGGTGTCCCTGAGCTCCCTGACCTCCGATAAACCCTCCC |
| | | GCCATTCCGCCGCTGCCGATGATGACGGCGATAAGTGGAGCCTGC |
| | | TGCTGAACGGCCGCGATGAAGAACAGTATATCTAAGGAATTCGAT |
| | | ATCAAGCTTATCGATAATCAACCTCTGGATTACAAAATTTGTGAAA |
| | | GATTGACTGGTATTCTTAACTATGTTGCTCCTTTTACGCTATGTGGA |
| | | TACGCTGCTTTAATGCCTTTGTATCATGCTATTGCTTCCCGTATGGC |
| | | TTTCATTTTCTCCTCCTTGTATAAATCCTGGTTGCTGTCTCTTTATGA |
| | | GGAGTTGTGGCCCGTTGTCAGGCAACGTGGCGTGGTGTGCACTGTG |
| | | TTTGCTGACGCAACCCCCACTGGTTGGGGCACCTGCAGGAATAAA |
| | | AGATCTTTATTTTCATTAGATCTGTGTGTTGGTTTTTTGTGTGTCTA |
| | | GAGTAGATAAGTAGCATGGCGGGTTAATCATTAACTACAAGGAAC |
| | | CCCTAGTGATGGAGTTGGCCACTCCCTCTCTGCGCGCTCGCTCGCT |
| | | CACTGAGGCCGGGCGACCAAAGGTCGCCCGACGCCCGGGCTTTGC |
| | | CCGGGCGGCCTCAGTGAGCGAGCGAGCGCGCAGAGAGGGAGTGG |
| | | CCAA |
| 77 | A24 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCAACTCCTAAAAAACC |
| | | GCCACCATGCCCGAACAGGAAAGACAGATTACCGCCCGCGAGGGC |
| | | GCTTCCCGCAAAATTCTGAGCAAACTGAGCCTGCCCACCCGCGCCT |
| | | GGGAACCTGCCATGAAGAAAAGCTTTGCTTTTGATAATGTCGGCTA |
| | | TGAGGGCGGCCTGGACGGCCTGGGCCCTAGCAGCCAGGTGGCTAC |
| | | CACCCTGATTGCCATTGCCGGCATGACCTGCGCCAGCTGCGTGCAT |
| | | AGCATTGAGGGCATGATTAGCCAGCTGGAGGGCGTGCAGCAGATT |
| | | TCCGTGAGCCTGGCTGAGGGCACCGCTACCGTGCTGTATAATCCCT |
| | | CCGTGATTAGCCCCGAAGAACTGCGCGCTGCCATTGAAGATATGG |
| | | GCTTTGAAGCCTCCGTGGTGTCCGAAAGCTGCAGCACCAATCCCCT |
| | | GGGCAATCATTCCGCCGGCAATAGCATGGTGCAGACCACCGACGG |
| | | CACCCCCACCTCCGTGCAGGAAGTGGCTCCCCATACCGGCCGCCTG |
| | | CCCGCCAATCATGCCCCTGATATTCTGGCTAAAAGCCCCCAGAGCA |
| | | CCCGCGCCGTGGCTCCTCAGAAATGCTTTCTGCAGATTAAGGGCAT |
| | | GACCTGCGCTAGCTGCGTGAGCAATATTGAACGCAATCTGCAGAA |
| | | AGAAGCCGGCGTGCTGTCCGTGCTGGTGGCCCTGATGGCCGGCAA |
| | | AGCTGAAATCAAATATGATCCCGAAGTGATTCAGCCCCTGGAAAT |
| | | TGCCCAGTTCATTCAGGATCTGGGCTTTGAGGCTGCCGTGATGGAA |
| | | GATTATGCCGGCTCCGACGGCAATATTGAACTGACCATTACCGGCA |
| | | TGACCTGCGCCAGCTGCGTGCATAATATTGAAAGCAAACTGACCC |
| | | GCACCAACGGCATTACCTATGCTTCCGTGGCCCTGGCTACCAGCAA |
| | | AGCCCTGGTGAAATTTGATCCTGAAATTATCGGCCCCCGCGATATT |
| | | ATCAAAATTATTGAAGAAATCGGCTTTCACGCCAGCCTGGCTCAGC |
| | | GCAATCCCAATGCCCATCATCTGGATCATAAGATGGAAATCAAAC |
| | | AGTGGAAGAAAAGCTTTCTGTGCAGCCTGGTGTTCGGCATTCCTGT |
| | | GATGGCCCTGATGATCTATATGCTGATTCCCAGCAATGAACCCCAT |
| | | CAGAGCATGGTGCTGGATCATAATATTATTCCCGGCCTGAGCATTC |
| | | TGAATCTGATTTTCTTTATCCTGTGCACCTTTGTGCAGCTGCTGGGC |
| | | GGCTGGTATTTCTATGTGCAGGCTTATAAAAGCCTGCGCCACCGCT |
| | | CCGCCAATATGGATGTGCTGATTGTGCTGGCCACCAGCATTGCTTA |
| | | TGTCTATAGCCTGGTCATTCTGGTGGTGGCCGTGGCCGAGAAAGCT |
| | | GAACGCAGCCCCGTGACCTTCTTTGATACCCCTCCCATGCTGTTCG |
| | | TGTTTATCGCTCTGGGCCGCTGGCTGGAACATCTGGCCAAAAGCAA |
| | | AACCTCCGAAGCCCTGGCTAAACTGATGAGCCTGCAGGCCACCGA |
| | | AGCCACCGTGGTGACCCTGGGCGAAGATAATCTGATTATCCGCGA |
| | | AGAACAGGTGCCTATGGAACTGGTGCAGCGCGGCGATATTGTGAA |
| | | AGTGGTGCCCGGCGGCAAATTTCCCGTGGACGGCAAAGTGCTGGA |
| | | GGGCAATACCATGGCTGATGAATCCCTGATTACCGGCGAAGCTAT |
| | | GCCTGTGACCAAGAAACCCGGCAGCACCGTGATTGCCGGCAGCAT |
| | | TAATGCTCACGGCTCCGTGCTGATCAAGGCTACCCATGTCGGCAAT |
| | | GATACCACCCTGGCCCAGATTGTGAAACTGGTGGAAGAAGCCCAG |
| | | ATGAGCAAAGCCCCTATTCAGCAGCTGGCTGACCGCTTTTCCGGCT |
| | | ATTTTGTGCCCTTTATCATTATTATGAGCACCCTGACCCTGGTGGTG |
| | | TGGATTGTGATCGGCTTCATTGATTTCGGCGTGGTGCAGAGATATT |
| | | TTCCTAATCCCAACAAACATATTAGCCAGACCGAAGTGATTATCCG |
| | | CTTTGCTTTTCAGACCAGCATTACCGTGCTGTGCATTGCTTGCCCCT |
| | | GCAGCCTGGGCCTGGCTACCCCCACCGCCGTCATGGTCGGCACCG |
| | | GCGTGGCTGCCCAGAACGGCATTCTGATTAAGGGCGGCAAACCTC |
| | | TGGAAATGGCCCACAAAATCAAAACCGTGATGTTTGATAAAACCG |
| | | GCACCATTACCCACGGCGTGCCCCGCGTGATGCGCGTGCTGCTGCT |
| | | GGGCGATGTGGCTACCCTGCCCCTGCGCAAAGTGCTGGCCGTGGTC |
| | | GGCACCGCCGAAGCTAGCTCCGAACATCCCCTGGGCGTGGCCGTG |
| | | ACCAAATATTGCAAAGAAGAACTGGGCACCGAAACCCTGGGCTAT |
| | | TGCACCGATTTTCAGGCCGTCCCTGGCTGCGGCATCGGCTGCAAAG |
| | | TGAGCAATGTGGAGGGCATTCTGGCCCATTCCGAAAGACCCCTGTC |
| | | CGCCCCTGCTAGCCATCTGAATGAAGCCGGCAGCCTGCCTGCTGAG |
| | | AAAGATGCCGTGCCCCAGACCTTTTCCGTGCTGATCGGCAACCGCG |
| | | AATGGCTGCGCCGCAACGGCCTGACCATTAGCTCCGATGTCTCCGA |
| | | TGCCATGACCGATCATGAAATGAAAGGCCAGACCGCCATTCTGGT |
| | | GGCCATTGACGGCGTGCTGTGCGGCATGATTGCCATTGCCGATGCC |
| | | GTGAAACAGGAGGCTGCCCTGGCTGTGCATACCCTGCAGAGCATG |
| | | GGCGTGGATGTGGTGCTGATTACCGGCGATAACCGCAAAACCGCT |
| | | CGCGCCATTGCCACCCAGGTCGGCATTAACAAAGTGTTTGCTGAAG |
| | | TGCTGCCCAGCCATAAAGTGGCTAAAGTGCAGGAACTGCAGAACA |
| | | AAGGCAAGAAAGTGGCTATGGTCGGCGACGGCGTGAATGATAGCC |
| | | CTGCCCTGGCCCAGGCCGATATGGGCGTGGCTATCGGCACCGGCA |
| | | CCGATGTGGCTATTGAGGCTGCCGATGTGGTGCTGATCCGCAATGA |
| | | TCTGCTGGATGTGGTGGCTAGCATTCATCTGAGCAAACGCACCGTC |
| | | CGCCGCATCCGCATTAATCTGGTGCTGGCCCTGATCTATAATCTGG |
| | | TCGGCATTCCCATCGCCGCCGGCGTGTTTATGCCCATCGGCATTGT |
| | | GCTGCAGCCCTGGATGGGCAGCGCCGCTATGGCCGCTAGCTCCGT |
| | | GTCCGTGGTGCTGAGCAGCCTGCAGCTGAAATGCTATAAGAAACC |
| | | CGATCTGGAAAGATATGAAGCTCAGGCTCACGGCCATATGAAACC |
| | | CCTGACCGCCAGCCAGGTGTCCGTGCATATCGGCATGGATGACCG |
| | | CTGGCGCGATAGCCCCCGCGCCACCCCTTGGGATCAGGTGAGCTAT |
| | | GTGAGCCAGGTGTCCCTGAGCTCCCTGACCTCCGATAAACCCTCCC |
| | | GCCATTCCGCCGCTGCCGATGATGACGGCGATAAGTGGAGCCTGC |
| | | TGCTGAACGGCCGCGATGAAGAACAGTATATCTAAGGAATTCGAT |
| | | ATCAAGCTTATCGATCACCTGCAGGAATAAAAGATCTTTATTTTCA |
| | | TTAGATCTGTGTGTTGGTTTTTTGTGTGTCTAGAGTAGATAAGTAG |
| | | CATGGCGGGTTAATCATTAACTACAAGGAACCCCTAGTGATGGAG |
| | | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAA |
| 78 | A25 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCCACCATGCCGGAACA |
| | | AGAACGCCAGATCACAGCAAGAGAAGGAGCAAGTAGGAAGATCC |
| | | TGAGCAAGCTCTCCCTGCCCACAAGGGCCTGGGAGCCGGCCATGA |
| | | AGAAGTCTTTTGCCTTCGATAATGTCGGTTATGAAGGCGGGCTCGA |
| | | TGGCCTGGGCCCCAGCAGCCAGCCCCAGAAGTGTTTCCTGCAGATC |
| | | AAGGGCATGACCTGTGCCAGCTGTGTTAGCAATATCGAAAGAAAC |
| | | CTCCAAAAAGAGGCTGGAGTTCTGAGTGTTCTAGTGGCTTTAATGG |
| | | CCGGGAAAGCCGAGATCAAGTATGATCCCGAGGTGATTCAGCCAT |
| | | TGGAGATTGCCCAGTTCATCCAGGATCTGGGCTTCGAGGCCGCCGT |
| | | GATGGAGGATTACGCCGGCTCTGATGGCAACATCGAACTGACCAT |
| | | CACCGGCATGACCTGTGCTTCCTGCGTGCATAATATCGAGAGCAAA |
| | | CTGACGCGGACCAATGGAATCACCTATGCCAGCGTCGCCCTGGCA |
| | | ACCTCTAAGGCTCTGGTGAAGTTTGACCCCGAAATCATTGGACCTA |
| | | GAGACATCATCAAAATTATTGAAGAGATTGGTTTCCACGCCAGCTT |
| | | AGCTCAGCGGAATCCCAACGCACACCATCTTGACCATAAGATGGA |
| | | AATCAAACAGTGGAAGAAGAGCTTCTTGTGTAGCCTGGTCTTCGG |
| | | GATACCCGTCATGGCACTGATGATTTACATGCTCATTCCGTCCAAC |
| | | GAGCCCCACCAGTCCATGGTGCTGGACCACAACATCATCCCTGGG |
| | | CTGTCTATCCTCAACCTGATCTTCTTTATTCTGTGCACATTTGTCCA |
| | | GCTCTTGGGAGGCTGGTATTTCTATGTACAGGCCTACAAATCCCTG |
| | | AGGCATCGAAGCGCCAACATGGACGTTTTAATTGTGCTGGCTACAT |
| | | CCATTGCCTACGTGTACTCCCTGGTGATCTTAGTGGTTGCTGTGGCT |
| | | GAAAAGGCTGAGAGAAGCCCTGTCACCTTCTTCGACACCCCCCCC |
| | | ATGCTCTTCGTGTTTATTGCACTGGGTCGCTGGCTGGAGCATTTAG |
| | | CCAAGTCCAAGACATCCGAGGCTCTGGCCAAGCTGATGTCTCTACA |
| | | GGCCACGGAGGCCACCGTGGTGACTCTGGGTGAAGACAATTTGAT |
| | | AATACGCGAGGAACAGGTGCCTATGGAGCTTGTGCAGCGCGGAGA |
| | | TATCGTCAAGGTGGTGCCCGGCGGCAAGTTCCCAGTGGATGGAAA |
| | | AGTGTTAGAGGGCAATACCATGGCCGATGAATCCCTCATCACTGG |
| | | CGAGGCAATGCCGGTGACCAAGAAACCTGGAAGTACTGTCATTGC |
| | | AGGCAGCATCAATGCCCATGGGAGCGTCCTGATCAAGGCCACCCA |
| | | CGTGGGCAATGATACAACTCTCGCCCAGATCGTCAAGCTAGTGGA |
| | | AGAGGCTCAGATGTCTAAAGCTCCAATCCAGCAACTGGCTGACCG |
| | | CTTCAGCGGCTACTTTGTCCCCTTTATCATCATCATGAGCACCCTCA |
| | | CCCTGGTGGTGTGGATAGTCATAGGCTTCATTGACTTTGGCGTGGT |
| | | GCAGAGATACTTCCCCAACCCTAACAAACATATCTCCCAAACCGA |
| | | GGTCATTATCAGATTCGCCTTTCAGACATCTATCACTGTGCTGTGC |
| | | ATAGCCTGCCCTTGCTCTCTGGGCCTGGCCACCCCCACCGCCGTGA |
| | | TGGTTGGAACCGGGGTCGCTGCACAAAATGGGATCCTGATAAAAG |
| | | GGGGCAAACCCCTGGAAATGGCACATAAGATTAAAACAGTTATGT |
| | | TTGATAAGACAGGCACCATTACTCACGGTGTCCCTCGAGTCATGAG |
| | | AGTGCTGCTGCTGGGGGACGTGGCTACACTGCCACTGCGCAAGGT |
| | | GCTGGCTGTGGTGGGCACAGCGGAGGCCTCCTCAGAACACCCATT |
| | | AGGCGTAGCTGTCACAAAATACTGTAAGGAGGAGCTGGGGACAGA |
| | | GACCCTCGGCTATTGTACAGACTTCCAGGCCGTGCCTGGCTGCGGC |
| | | ATCGGCTGTAAGGTCAGCAATGTGGAGGGCATCCTGGCACACAGC |
| | | GAGAGACCCCTCTCTGCCCCAGCCTCCCACCTGAATGAAGCTGGGT |
| | | CTCTGCCTGCTGAGAAGGACGCCGTCCCTCAGACCTTCTCCGTGTT |
| | | GATTGGGAACAGAGAGTGGCTTAGAAGGAATGGCCTGACCATCAG |
| | | CTCTGACGTCTCGGATGCCATGACAGATCACGAGATGAAAGGCCA |
| | | GACAGCCATACTGGTTGCCATCGACGGCGTGCTCTGTGGCATGATT |
| | | GCCATCGCTGATGCTGTTAAACAGGAAGCTGCGCTGGCAGTCCAC |
| | | ACCCTCCAGTCCATGGGAGTGGACGTGGTCCTGATTACTGGAGAC |
| | | AACAGGAAAACCGCTCGGGCCATCGCTACCCAAGTGGGGATAAAT |
| | | AAGGTTTTTGCAGAAGTCTTACCCTCCCACAAAGTAGCCAAAGTGC |
| | | AGGAGCTCCAGAACAAGGGTAAAAAGGTGGCAATGGTGGGAGAT |
| | | GGTGTGAATGACAGCCCAGCTCTCGCGCAGGCTGACATGGGCGTA |
| | | GCCATAGGTACTGGAACAGATGTCGCGATTGAAGCCGCTGACGTA |
| | | GTCCTCATCCGAAACGATTTGCTGGATGTGGTGGCCAGCATCCATC |
| | | TGTCCAAGCGTACCGTGCGCCGGATCCGCATCAACCTGGTCTTGGC |
| | | CCTCATCTATAATCTCGTGGGCATCCCCATCGCTGCTGGGGTCTTC |
| | | ATGCCCATCGGCATTGTGCTGCAGCCCTGGATGGGAAGCGCTGCC |
| | | ATGGCTGCTTCTTCTGTCTCCGTTGTGCTGTCCAGCCTACAGCTGAA |
| | | GTGCTATAAAAAGCCAGACCTCGAGCGCTACGAAGCCCAGGCGCA |
| | | TGGCCATATGAAGCCCCTCACGGCTTCCCAGGTCTCGGTTCACATT |
| | | GGGATGGATGACAGATGGCGGGACTCCCCACGCGCCACACCCTGG |
| | | GACCAGGTATCCTATGTGAGCCAGGTGAGCCTCAGCTCCCTGACAT |
| | | CAGATAAACCGTCTAGGCACTCAGCTGCCGCAGATGATGACGGTG |
| | | ACAAATGGAGCCTCCTCCTGAACGGCCGCGACGAGGAGCAGTACA |
| | | TCTAAGAATTCGATATCAAGCTTATCGATAATCAACCTCTGGATTA |
| | | CAAAATTTGTGAAAGATTGACTGGTATTCTTAACTATGTTGCTCCT |
| | | TTTACGCTATGTGGATACGCTGCTTTAATGCCTTTGTATCATGCTAT |
| | | TGCTTCCCGTATGGCTTTCATTTTCTCCTCCTTGTATAAATCCTGGT |
| | | TGCTGTCTCTTTATGAGGAGTTGTGGCCCGTTGTCAGGCAACGTGG |
| | | CGTGGTGTGCACTGTGTTTGCTGACGCAACCCCCACTGGTTGGGGC |
| | | ACCTGCAGGAATAAAAGATCTTTATTTTCATTAGATCTGTGTGTTG |
| | | GTTTTTTGTGTGTCTAGAGTAGATAAGTAGCATGGCGGGTTAATCA |
| | | TTAACTACAAGGAACCCCTAGTGATGGAGTTGGCCACTCCCTCTCT |
| | | GCGCGCTCGCTCGCTCACTGAGGCCGGGCGACCAAAGGTCGCCCG |
| | | ACGCCCGGGCTTTGCCCGGGCGGCCTCAGTGAGCGAGCGAGCGCG |
| | | CAGAGAGGGAGTGGCCAA |
| 79 | A26 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCCACCATGGCGGCGCC |
| | | TGAGCAGGAGAGACAGATCACAGCCAGAGAAGGGGCCAGTCGGA |
| | | AAATCTTATCTAAGCTTTCTTTGCCTACCCGTGCCTGGGAACCAGC |
| | | AATGAAGAAGAGTTTTGCTTTTGACAATGTTGGCTATGAAGGTGGT |
| | | CTGGATGGCCTGGGCCCTTCTTCTCAGGTGGCCCCTGATGGAGCCG |
| | | AAGGGAGTGGGACAGATCACAGGTCTTCCAGTTCTCATTCCCCTGG |
| | | CTCCCCACCGAGAAACCAGGTCCAGGGCACATGCAGTAAGTGCTT |
| | | CTTACAGATCAAAGGCATGACCTGTGCATCCTGTGTGTCTAACATA |
| | | GAAAGGAATCTGCAGAAAGAAGCTGGTGTTCTCTCCGTGTTGGTTG |
| | | CCTTGATGGCAGGAAAGGCAGAGATCAAGTATGACCCAGAGGTCA |
| | | TCCAGCCCCTCGAGATAGCTCAGTTCATCCAGGACCTGGGTTTTGA |
| | | GGCAGCAGTCATGGAGGACTACGCAGGCTCCGATGGCAACATTGA |
| | | GCTGACAATCACAGGGATGACCTGCGCGTCCTGTGTCCACAACAT |
| | | AGAGTCCAAACTCACGAGGACAAATGGCATCACTTATGCCTCCGTT |
| | | GCCCTTGCCACCAGCAAAGCCCTTGTTAAGTTTGACCCGGAAATTA |
| | | TCGGTCCACGGGATATTATCAAAATTATTGAGGAAATTGGCTTTCA |
| | | TGCTTCCCTGGCCCAGAGAAACCCCAACGCTCATCACTTGGACCAC |
| | | AAGATGGAAATAAAGCAGTGGAAGAAGTCTTTTCTTGTGAGTCTG |
| | | TTTTTCTGTATTCCTGTAATGGGGCTGATGATATATATGATGGTTAT |
| | | GGACCACCACTTTGCAACTCTTCACCATAATCAAAACATGAGTAAA |
| | | GAAGAAATGATCAACCTTCATTCTTCTATGTTCCTGGAGCGCCAGA |
| | | TTCTTCCAGGATTGTCTGTTATGAATTTGCTGTCCTTTTTATTGTGT |
| | | GTACCTGTACAGTTTTTCGGAGGCTGGTACTTCTACATTCAGGCTT |
| | | ATAAAGCACTGAAGCATAAGACAGCAAATATGGACGTACTGATTG |
| | | TGCTGGCAACCACCATTGCATTTGCCTACTCTTTGATTATTCTTCTA |
| | | GTTGCAATGTATGAGAGAGCCAAAGTGAACCCTATTACTTTCTTTG |
| | | ACACACCCCCTATGCTGTTTGTGTTTATTGCACTAGGCCGATGGCT |
| | | GGAACATATAGCAAAGGGCAAAACATCAGAGGCTCTTGCAAAGTT |
| | | AATTTCACTACAAGCTACAGAAGCAACTATTGTAACTCTTGATTCT |
| | | GATAATATCCTCCTCAGTGAAGAACAAGTGGATGTGGAACTTGTA |
| | | CAACGTGGAGATATCATTAAAGTAGTTCCAGGAGGCAAATTTCCA |
| | | GTGGATGGTCGTGTTATTGAAGGACATTCTATGGTAGATGAGTCCC |
| | | TCATCACAGGGGAGGCAATGCCTGTGGCTAAGAAACCTGGCAGCA |
| | | CAGTGATTGCTGGTTCCATTAACCAGAACGGGTCACTGCTTATCTG |
| | | CGCAACACATGTTGGAGCAGACACAACCCTTTCTCAAATTGTCAAA |
| | | CTTGTGGAAGAGGCACAAACATCAAAGGCTCCTATCCAGCAGTTT |
| | | GCAGACAAACTCAGTGGCTATTTTGTTCCTTTTATTGTTTTTGTTTC |
| | | CATTGCCACCCTCTTGGTATGGATTGTAATTGGATTTCTGAATTTTG |
| | | AAATTGTGGAAACCTACTTTCCTGGCTACAATAGAAGTATCTCCCG |
| | | AACAGAAACGATAATACGATTTGCTTTCCAAGCCTCTATCACAGTT |
| | | CTGTGTATTGCATGTCCCTGTTCACTGGGACTGGCCACTCCAACTG |
| | | CTGTGATGGTGGGTACAGGAGTAGGTGCTCAAAATGGCATACTAA |
| | | TAAAAGGTGGAGAGCCATTGGAGATGGCTCATAAGGTAAAGGTAG |
| | | TGGTATTTGATAAGACTGGAACCATTACTCACGGAACCCCAGTGGT |
| | | GAATCAAGTAAAGGTTCTAACTGAAAGTAACAGAATATCACACCA |
| | | TAAAATCTTGGCCATTGTGGGAACTGCTGAAAGTAACAGTGAACA |
| | | CCCTCTAGGAACAGCCATAACCAAATATTGCAAACAGGAGCTGGA |
| | | CACTGAAACCTTGGGTACCTGCATAGATTTCCAGGTTGTGCCAGGC |
| | | TGTGGTATTAGCTGTAAAGTCACCAATATTGAAGGCTTGCTACATA |
| | | AGAATAACTGGAATATAGAGGACAATAATATTAAAAATGCATCCC |
| | | TGGTTCAAATTGATGCCAGTAATGAACAGTCATCAACTTCGTCTTC |
| | | CATGATTATTGATGCCCAGATCTCAAATGCTCTTAATGCTCAGCAG |
| | | TATAAAGTCCTCATTGGTAACCGGGAGTGGATGATTAGAAATGGT |
| | | CTTGTCATTAATAACGATGTAAATGATTTCATGACTGAACATGAGA |
| | | GAAAAGGTCGGACTGCTGTATTAGTAGCAGTTGATGATGAGCTGT |
| | | GTGGCTTGATAGCCATTGCAGACACAGTGAAGCCTGAAGCAGAAC |
| | | TGGCTATCCATATTCTGAAATCTATGGGCTTAGAAGTAGTTCTGAT |
| | | GACTGGAGACAACAGTAAAACAGCTAGATCTATTGCTTCTCAGGTT |
| | | GGCATTACTAAGGTGTTTGCTGAAGTTCTACCTTCTCACAAGGTTG |
| | | CTAAAGTGAAGCAACTTCAAGAGGAGGGGAAACGGGTAGCAATG |
| | | GTGGGAGATGGAATCAATGACTCCCCAGCTCTGGCAATGGCTAAT |
| | | GTGGGAATTGCTATTGGCACAGGCACAGATGTAGCCATTGAAGCA |
| | | GCTGATGTGGTTTTGATAAGGAATGATCTTCTGGATGTAGTGGCAA |
| | | GTATTGACTTATCAAGAAAGACAGTCAAGAGGATTCGGATAAATT |
| | | TTGTCTTTGCTCTAATTTATAATCTGGTTGGAATTCCCATAGCTGCT |
| | | GGAGTTTTTATGCCCATTGGTTTGGTTTTGCAGCCCTGGATGGGAT |
| | | CTGCAGCAATGGCTGCTTCATCTGTTTCTGTAGTACTTTCTTCTCTC |
| | | CAGCTCAAGTGCTATAAGAAGCCTGACCTGGAGAGGTATGAGGCA |
| | | CAGGCGCATGGCCACATGAAGCCCCTGACGGCATCCCAGGTCAGT |
| | | GTGCACATAGGCATGGATGACAGGTGGCGGGACTCCCCCAGGGCC |
| | | ACACCATGGGACCAGGTCAGCTATGTCAGCCAGGTGTCGCTGTCCT |
| | | CCCTGACGTCCGACAAGCCATCTCGGCACAGCGCTGCAGCAGACG |
| | | ATGATGGGGACAAGTGGTCTCTGCTCCTGAATGGCAGGGATGAGG |
| | | AGCAGTACATCTGAGAATTCGATATCAAGCTTATCGATAATCAACC |
| | | TCTGGATTACAAAATTTGTGAAAGATTGACTGGTATTCTTAACTAT |
| | | GTTGCTCCTTTTACGCTATGTGGATACGCTGCTTTAATGCCTTTGTA |
| | | TCATGCTATTGCTTCCCGTATGGCTTTCATTTTCTCCTCCTTGTATA |
| | | AATCCTGGTTGCTGTCTCTTTATGAGGAGTTGTGGCCCGTTGTCAG |
| | | GCAACGTGGCGTGGTGTGCACTGTGTTTGCTGACGCAACCCCCACT |
| | | GGTTGGGGCACCTGCAGGAATAAAAGATCTTTATTTTCATTAGATC |
| | | TGTGTGTTGGTTTTTTGTGTGTCTAGAGTAGATAAGTAGCATGGCG |
| | | GGTTAATCATTAACTACAAGGAACCCCTAGTGATGGAGTTGGCCA |
| | | CTCCCTCTCTGCGCGCTCGCTCGCTCACTGAGGCCGGGCGACCAAA |
| | | GGTCGCCCGACGCCCGGGCTTTGCCCGGGCGGCCTCAGTGAGCGA |
| | | GCGAGCGCGCAGAGAGGGAGTGGCCAA |
| 80 | A27 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCCACCATGCCGGAACA |
| | | AGAACGCCAGATCACAGCAAGAGAAGGAGCAAGTAGGAAGATCC |
| | | TGAGCAAGCTCTCCCTGCCCACAAGGGCCTGGGAGCCGGCCATGA |
| | | AGAAGTCTTTTGCCTTCGATAATGTCGGTTATGAAGGCGGGCTCGA |
| | | TGGCCTGGGCCCCAGCAGCCAGGTGGCGACCACCTTAATCGCCAT |
| | | CGCAGGCATGACCTGCGCATCTTGTGTGCATAGCATTGAAGGCATG |
| | | ATTAGCCAGCTGGAGGGCGTGCAGCAGATCTCAGTCTCCCTGGCTG |
| | | AGGGGACCGCCACTGTCTTGTACAATCCGTCTGTTATTTCCCCGGA |
| | | GGAGCTGAGAGCTGCCATTGAGGACATGGGATTTGAAGCATCGGT |
| | | GGTGTCTGAGTCATGCTCCACCAATCCCCTGGGAAATCACTCAGCT |
| | | GGCAATTCAATGGTTCAGACCACCGATGGGACCCCAACCAGCGTG |
| | | CAGGAAGTGGCACCCCATACCGGCAGACTGCCTGCCAATCATGCT |
| | | CCTGATATCCTCGCCAAGAGCCCCCAGAGCACCCGCGCGGTGGCA |
| | | CCCCAGAAGTGTTTCCTGCAGATCAAGGGCATGACCTGTGCCAGCT |
| | | GTGTTAGCAATATCGAAAGAAACCTCCAAAAAGAGGCTGGAGTTC |
| | | TGAGTGTTCTAGTGGCTTTAATGGCCGGGAAAGCCGAGATCAAGT |
| | | ATGATCCCGAGGTGATTCAGCCATTGGAGATTGCCCAGTTCATCCA |
| | | GGATCTGGGCTTCGAGGCCGCCGTGATGGAGGATTACGCCGGCTC |
| | | TGATGGCAACATCGAACTGACCATCACCGGCATGACCTGTGCTTCC |
| | | TGCGTGCATAATATCGAGAGCAAACTGACGCGGACCAATGGAATC |
| | | ACCTATGCCAGCGTCGCCCTGGCAACCTCTAAGGCTCTGGTGAAGT |
| | | TTGACCCCGAAATCATTGGACCTAGAGACATCATCAAAATTATTGA |
| | | AGAGATTGGTTTCCACGCCAGCTTAGCTCAGCGGAATCCCAACGC |
| | | ACACCATCTTGACCATAAGATGGAAATCAAACAGTGGAAGAAGAG |
| | | CTTCTTGTGTAGCCTGGTCTTCGGGATACCCGTCATGGCACTGATG |
| | | ATTTACATGCTCATTCCGTCCAACGAGCCCCACCAGTCCATGGTGC |
| | | TGGACCACAACATCATCCCTGGGCTGTCTATCCTCAACCTGATCTT |
| | | CTTTATTCTGTGCACATTTGTCCAGCTCTTGGGAGGCTGGTATTTCT |
| | | ATGTACAGGCCTACAAATCCCTGAGGCATCGAAGCGCCAACATGG |
| | | ACGTTTTAATTGTGCTGGCTACATCCATTGCCTACGTGTACTCCCTG |
| | | GTGATCTTAGTGGTTGCTGTGGCTGAAAAGGCTGAGAGAAGCCCT |
| | | GTCACCTTCTTCGACACCCCCCCCATGCTCTTCGTGTTTATTGCACT |
| | | GGGTCGCTGGCTGGAGCATTTAGCCAAGTCCAAGACATCCGAGGC |
| | | TCTGGCCAAGCTGATGTCTCTACAGGCCACGGAGGCCACCGTGGT |
| | | GACTCTGGGTGAAGACAATTTGATAATACGCGAGGAACAGGTGCC |
| | | TATGGAGCTTGTGCAGCGCGGAGATATCGTCAAGGTGGTGCCCGG |
| | | CGGCAAGTTCCCAGTGGATGGAAAAGTGTTAGAGGGCAATACCAT |
| | | GGCCGATGAATCCCTCATCACTGGCGAGGCAATGCCGGTGACCAA |
| | | GAAACCTGGAAGTACTGTCATTGCAGGCAGCATCAATGCCCATGG |
| | | GAGCGTCCTGATCAAGGCCACCCACGTGGGCAATGATACAACTCT |
| | | CGCCCAGATCGTCAAGCTAGTGGAAGAGGCTCAGATGTCTAAAGC |
| | | TCCAATCCAGCAACTGGCTGACCGCTTCAGCGGCTACTTTGTCCCC |
| | | TTTATCATCATCATGAGCACCCTCACCCTGGTGGTGTGGATAGTCA |
| | | TAGGCTTCATTGACTTTGGCGTGGTGCAGAGATACTTCCCCAACCC |
| | | TAACAAACATATCTCCCAAACCGAGGTCATTATCAGATTCGCCTTT |
| | | CAGACATCTATCACTGTGCTGTGCATAGCCTGCCCTTGCTCTCTGG |
| | | GCCTGGCCACCCCCACCGCCGTGATGGTTGGAACCGGGGTCGCTG |
| | | CACAAAATGGGATCCTGATAAAAGGGGGCAAACCCCTGGAAATGG |
| | | CACATAAGATTAAAACAGTTATGTTTGATAAGACAGGCACCATTA |
| | | CTCACGGTGTCCCTCGAGTCATGAGAGTGCTGCTGCTGGGGGACGT |
| | | GGCTACACTGCCACTGCGCAAGGTGCTGGCTGTGGTGGGCACAGC |
| | | GGAGGCCTCCTCAGAACACCCATTAGGCGTAGCTGTCACAAAATA |
| | | CTGTAAGGAGGAGCTGGGGACAGAGACCCTCGGCTATTGTACAGA |
| | | CTTCCAGGCCGTGCCTGGCTGCGGCATCGGCTGTAAGGTCAGCAAT |
| | | GTGGAGGGCATCCTGGCACACAGCGAGAGACCCCTCTCTGCCCCA |
| | | GCCTCCCACCTGAATGAAGCTGGGTCTCTGCCTGCTGAGAAGGAC |
| | | GCCGTCCCTCAGACCTTCTCCGTGTTGATTGGGAACAGAGAGTGGC |
| | | TTAGAAGGAATGGCCTGACCATCAGCTCTGACGTCTCGGATGCCAT |
| | | GACAGATCACGAGATGAAAGGCCAGACAGCCATACTGGTTGCCAT |
| | | CGACGGCGTGCTCTGTGGCATGATTGCCATCGCTGATGCTGTTAAA |
| | | CAGGAAGCTGCGCTGGCAGTCCACACCCTCCAGTCCATGGGAGTG |
| | | GACGTGGTCCTGATTACTGGAGACAACAGGAAAACCGCTCGGGCC |
| | | ATCGCTACCCAAGTGGGGATAAATAAGGTTTTTGCAGAAGTCTTAC |
| | | CCTCCCACAAAGTAGCCAAAGTGCAGGAGCTCCAGAACAAGGGTA |
| | | AAAAGGTGGCAATGGTGGGAGATGGTGTGAATGACAGCCCAGCTC |
| | | TCGCGCAGGCTGACATGGGCGTAGCCATAGGTACTGGAACAGATG |
| | | TCGCGATTGAAGCCGCTGACGTAGTCCTCATCCGAAACGATTTGCT |
| | | GGATGTGGTGGCCAGCATCCATCTGTCCAAGCGTACCGTGCGCCG |
| | | GATCCGCATCAACCTGGTCTTGGCCCTCATCTATAATCTCGTGGGC |
| | | ATCCCCATCGCTGCTGGGGTCTTCATGCCCATCGGCATTGTGCTGC |
| | | AGCCCTGGATGGGAAGCGCTGCCATGGCTGCTTCTTCTGTCTCCGT |
| | | TGTGCTGTCCAGCCTACAGCTGAAGTGCTATAAAAAGCCAGACCTC |
| | | GAGCGCTACGAAGCCCAGGCGCATGGCCATATGAAGCCCCTCACG |
| | | GCTTCCCAGGTCTCGGTTCACATTGGGATGGATGACAGATGGCGG |
| | | GACTCCCCACGCGCCACACCCTGGGACCAGGTATCCTATGTGAGCC |
| | | AGGTGAGCCTCAGCTCCCTGACATCAGATAAACCGTCTAGGCACTC |
| | | AGCTGCCGCAGATGATGACGGTGACAAATGGAGCCTCCTCCTGAA |
| | | CGGCCGCGACGAGGAGCAGTACATCTAAGAATTCGATATCAAGCT |
| | | TATCGATAATCAACCTCTGGATTACAAAATTTGTGAAAGATTGACT |
| | | GGTATTCTTAACTATGTTGCTCCTTTTACGCTATGTGGATACGCTGC |
| | | TTTAATGCCTTTGTATCATGCTATTGCTTCCCGTATGGCTTTCATTT |
| | | TCTCCTCCTTGTATAAATCCTGGTTGCTGTCTCTTTATGAGGAGTTG |
| | | TGGCCCGTTGTCAGGCAACGTGGCGTGGTGTGCACTGTGTTTGCTG |
| | | ACGCAACCCCCACTGGTTGGGGCACCTGCAGGAATAAAAGATCTT |
| | | TATTTTCATTAGATCTGTGTGTTGGTTTTTTGTGTGTCTAGAGTAGA |
| | | TAAGTAGCATGGCGGGTTAATCATTAACTACAAGGAACCCCTAGT |
| | | GATGGAGTTGGCCACTCCCTCTCTGCGCGCTCGCTCGCTCACTGAG |
| | | GCCGGGCGACCAAAGGTCGCCCGACGCCCGGGCTTTGCCCGGGCG |
| | | GCCTCAGTGAGCGAGCGAGCGCGCAGAGAGGGAGTGGCCAA |
| 81 | A28 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTAATAATTACCAGCGCGGGCCAAAT |
| | | AAATAATCCGCGAGGGGCAGCTGGACTTTGGACTCCGCGCGCTGG |
| | | TAATTATTAACCTCGCGAATATTGATTCGAGGCCGCGATTGCCGCA |
| | | ATCGCGAGGGGCAGGTGACCTTTGCCCAGCGCGCGTTCGCCCCGC |
| | | CCCGGACGGTATCGATAAGCTTAGGAGCTTGGGCTGCAGGTCGAG |
| | | GGCACTGGGAGGATGTTGAGTAAGATGGAAAACTACTGATGACCC |
| | | TTGCAGAGACAGAGTATTAGGACATGTTTGAACAGGGGCCGGGCG |
| | | ATCAGCAGGTAGCTCTAGAGGATCCCCGTCTGTCTGCACATTTCGT |
| | | AGAGCGAGTGTTCCGATACTCTAATCTCCCTAGGCAAGGTTCATAT |
| | | TTGTGTAGGTTACTTATTCTCCTTTTGTTGACTAAGTCAATAATCAG |
| | | AATCAGCAGGTTTGGAGTCAGCTTGGCAGGGATCAGCAGCCTGGG |
| | | TTGGAAGGAGGGGGTATAAAAGCCCCTTCACCAGGAGAAGCCGTC |
| | | ACACAGATCCACAAGCTCGAGCCGAGTGGTCGTGCCTCCATAGAA |
| | | GCGGCCGCCACCATGCCGGAACAAGAACGCCAGATCACAGCAAGA |
| | | GAAGGAGCAAGTAGGAAGATCCTGAGCAAGCTCTCCCTGCCCACA |
| | | AGGGCCTGGGAGCCGGCCATGAAGAAGTCTTTTGCCTTCGATAAT |
| | | GTCGGTTATGAAGGCGGGCTCGATGGCCTGGGCCCCAGCAGCCAG |
| | | CCCCAGAAGTGTTTCCTGCAGATCAAGGGCATGACCTGTGCCAGCT |
| | | GTGTTAGCAATATCGAAAGAAACCTCCAAAAAGAGGCTGGAGTTC |
| | | TGAGTGTTCTAGTGGCTTTAATGGCCGGGAAAGCCGAGATCAAGT |
| | | ATGATCCCGAGGTGATTCAGCCATTGGAGATTGCCCAGTTCATCCA |
| | | GGATCTGGGCTTCGAGGCCGCCGTGATGGAGGATTACGCCGGCTC |
| | | TGATGGCAACATCGAACTGACCATCACCGGCATGACCTGTGCTTCC |
| | | TGCGTGCATAATATCGAGAGCAAACTGACGCGGACCAATGGAATC |
| | | ACCTATGCCAGCGTCGCCCTGGCAACCTCTAAGGCTCTGGTGAAGT |
| | | TTGACCCCGAAATCATTGGACCTAGAGACATCATCAAAATTATTGA |
| | | AGAGATTGGTTTCCACGCCAGCTTAGCTCAGCGGAATCCCAACGC |
| | | ACACCATCTTGACCATAAGATGGAAATCAAACAGTGGAAGAAGAG |
| | | CTTCTTGTGTAGCCTGGTCTTCGGGATACCCGTCATGGCACTGATG |
| | | ATTTACATGCTCATTCCGTCCAACGAGCCCCACCAGTCCATGGTGC |
| | | TGGACCACAACATCATCCCTGGGCTGTCTATCCTCAACCTGATCTT |
| | | CTTTATTCTGTGCACATTTGTCCAGCTCTTGGGAGGCTGGTATTTCT |
| | | ATGTACAGGCCTACAAATCCCTGAGGCATCGAAGCGCCAACATGG |
| | | ACGTTTTAATTGTGCTGGCTACATCCATTGCCTACGTGTACTCCCTG |
| | | GTGATCTTAGTGGTTGCTGTGGCTGAAAAGGCTGAGAGAAGCCCT |
| | | GTCACCTTCTTCGACACCCCCCCCATGCTCTTCGTGTTTATTGCACT |
| | | GGGTCGCTGGCTGGAGCATTTAGCCAAGTCCAAGACATCCGAGGC |
| | | TCTGGCCAAGCTGATGTCTCTACAGGCCACGGAGGCCACCGTGGT |
| | | GACTCTGGGTGAAGACAATTTGATAATACGCGAGGAACAGGTGCC |
| | | TATGGAGCTTGTGCAGCGCGGAGATATCGTCAAGGTGGTGCCCGG |
| | | CGGCAAGTTCCCAGTGGATGGAAAAGTGTTAGAGGGCAATACCAT |
| | | GGCCGATGAATCCCTCATCACTGGCGAGGCAATGCCGGTGACCAA |
| | | GAAACCTGGAAGTACTGTCATTGCAGGCAGCATCAATGCCCATGG |
| | | GAGCGTCCTGATCAAGGCCACCCACGTGGGCAATGATACAACTCT |
| | | CGCCCAGATCGTCAAGCTAGTGGAAGAGGCTCAGATGTCTAAAGC |
| | | TCCAATCCAGCAACTGGCTGACCGCTTCAGCGGCTACTTTGTCCCC |
| | | TTTATCATCATCATGAGCACCCTCACCCTGGTGGTGTGGATAGTCA |
| | | TAGGCTTCATTGACTTTGGCGTGGTGCAGAGATACTTCCCCAACCC |
| | | TAACAAACATATCTCCCAAACCGAGGTCATTATCAGATTCGCCTTT |
| | | CAGACATCTATCACTGTGCTGTGCATAGCCTGCCCTTGCTCTCTGG |
| | | GCCTGGCCACCCCCACCGCCGTGATGGTTGGAACCGGGGTCGCTG |
| | | CACAAAATGGGATCCTGATAAAAGGGGGCAAACCCCTGGAAATGG |
| | | CACATAAGATTAAAACAGTTATGTTTGATAAGACAGGCACCATTA |
| | | CTCACGGTGTCCCTCGAGTCATGAGAGTGCTGCTGCTGGGGGACGT |
| | | GGCTACACTGCCACTGCGCAAGGTGCTGGCTGTGGTGGGCACAGC |
| | | GGAGGCCTCCTCAGAACACCCATTAGGCGTAGCTGTCACAAAATA |
| | | CTGTAAGGAGGAGCTGGGGACAGAGACCCTCGGCTATTGTACAGA |
| | | CTTCCAGGCCGTGCCTGGCTGCGGCATCGGCTGTAAGGTCAGCAAT |
| | | GTGGAGGGCATCCTGGCACACAGCGAGAGACCCCTCTCTGCCCCA |
| | | GCCTCCCACCTGAATGAAGCTGGGTCTCTGCCTGCTGAGAAGGAC |
| | | GCCGTCCCTCAGACCTTCTCCGTGTTGATTGGGAACAGAGAGTGGC |
| | | TTAGAAGGAATGGCCTGACCATCAGCTCTGACGTCTCGGATGCCAT |
| | | GACAGATCACGAGATGAAAGGCCAGACAGCCATACTGGTTGCCAT |
| | | CGACGGCGTGCTCTGTGGCATGATTGCCATCGCTGATGCTGTTAAA |
| | | CAGGAAGCTGCGCTGGCAGTCCACACCCTCCAGTCCATGGGAGTG |
| | | GACGTGGTCCTGATTACTGGAGACAACAGGAAAACCGCTCGGGCC |
| | | ATCGCTACCCAAGTGGGGATAAATAAGGTTTTTGCAGAAGTCTTAC |
| | | CCTCCCACAAAGTAGCCAAAGTGCAGGAGCTCCAGAACAAGGGTA |
| | | AAAAGGTGGCAATGGTGGGAGATGGTGTGAATGACAGCCCAGCTC |
| | | TCGCGCAGGCTGACATGGGCGTAGCCATAGGTACTGGAACAGATG |
| | | TCGCGATTGAAGCCGCTGACGTAGTCCTCATCCGAAACGATTTGCT |
| | | GGATGTGGTGGCCAGCATCCATCTGTCCAAGCGTACCGTGCGCCG |
| | | GATCCGCATCAACCTGGTCTTGGCCCTCATCTATAATCTCGTGGGC |
| | | ATCCCCATCGCTGCTGGGGTCTTCATGCCCATCGGCATTGTGCTGC |
| | | AGCCCTGGATGGGAAGCGCTGCCATGGCTGCTTCTTCTGTCTCCGT |
| | | TGTGCTGTCCAGCCTACAGCTGAAGTGCTATAAAAAGCCAGACCTC |
| | | GAGCGCTACGAAGCCCAGGCGCATGGCCATATGAAGCCCCTCACG |
| | | GCTTCCCAGGTCTCGGTTCACATTGGGATGGATGACAGATGGCGG |
| | | GACTCCCCACGCGCCACACCCTGGGACCAGGTATCCTATGTGAGCC |
| | | AGGTGAGCCTCAGCTCCCTGACATCAGATAAACCGTCTAGGCACTC |
| | | AGCTGCCGCAGATGATGACGGTGACAAATGGAGCCTCCTCCTGAA |
| | | CGGCCGCGACGAGGAGCAGTACATCTAAGAATTCGATATCAAGCT |
| | | TATCGATAATCAACCTCTGGATTACAAAATTTGTGAAAGATTGACT |
| | | GGTATTCTTAACTATGTTGCTCCTTTTACGCTATGTGGATACGCTGC |
| | | TTTAATGCCTTTGTATCATGCTATTGCTTCCCGTATGGCTTTCATTT |
| | | TCTCCTCCTTGTATAAATCCTGGTTAGTTCTTGCCACGGCGGAACTC |
| | | ATCGCCGCCTGCCTTGCCCGCTGCTGGACAGGGGCTCGGCTGTTGG |
| | | GCACTGACAATTCCGTGGTGTTCACCTGCAGGAATAAAAGATCTTT |
| | | ATTTTCATTAGATCTGTGTGTTGGTTTTTTGTGTGTCTAGAGTAGAT |
| | | AAGTAGCATGGCGGGTTAATCATTAACTACAAGGAACCCCTAGTG |
| | | ATGGAGTTGGCCACTCCCTCTCTGCGCGCTCGCTCGCTCACTGAGG |
| | | CCGGGCGACCAAAGGTCGCCCGACGCCCGGGCTTTGCCCGGGCGG |
| | | CCTCAGTGAGCGAGCGAGCGCGCAGAGAGGGAGTGGCCAA |
| 82 | A29 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTAATAATTACCAGCGCGGGCCAAAT |
| | | AAATAATCCGCGAGGGGCAGCTGGACTTTGGACTCCGCGCGCTGG |
| | | TAATTATTAACCTCGCGAATATTGATTCGAGGCCGCGATTGCCGCA |
| | | ATCGCGAGGGGCAGGTGACCTTTGCCCAGCGCGCGTTCGCCCCGC |
| | | CCCGGACGGTATCGATAAGCTTAGGAGCTTGGGCTGCAGGTCGAG |
| | | GGCACTGGGAGGATGTTGAGTAAGATGGAAAACTACTGATGACCC |
| | | TTGCAGAGACAGAGTATTAGGACATGTTTGAACAGGGGCCGGGCG |
| | | ATCAGCAGGTAGCTCTAGAGGATCCCCGTCTGTCTGCACATTTCGT |
| | | AGAGCGAGTGTTCCGATACTCTAATCTCCCTAGGCAAGGTTCATAT |
| | | TTGTGTAGGTTACTTATTCTCCTTTTGTTGACTAAGTCAATAATCAG |
| | | AATCAGCAGGTTTGGAGTCAGCTTGGCAGGGATCAGCAGCCTGGG |
| | | TTGGAAGGAGGGGGTATAAAAGCCCCTTCACCAGGAGAAGCCGTC |
| | | ACACAGATCCACAAGCTCGAGCCGAGTGGTCGTGCCTCCATAGAA |
| | | GCGGCCGCAAGATGGCGGCGCCTGAGCAGGAGAGACAGATCACA |
| | | GCCAGAGAAGGGGCCAGTCGGAAAATCTTATCTAAGCTTTCTTTGC |
| | | CTACCCGTGCCTGGGAACCAGCAATGAAGAAGAGTTTTGCTTTTGA |
| | | CAATGTTGGCTATGAAGGTGGTCTGGATGGCCTGGGCCCCAGCAG |
| | | CCAGCCCCAGAAGTGTTTCCTGCAGATCAAGGGCATGACCTGTGCC |
| | | AGCTGTGTTAGCAATATCGAAAGAAACCTCCAAAAAGAGGCTGGA |
| | | GTTCTGAGTGTTCTAGTGGCTTTAATGGCCGGGAAAGCCGAGATCA |
| | | AGTATGATCCCGAGGTGATTCAGCCATTGGAGATTGCCCAGTTCAT |
| | | CCAGGATCTGGGCTTCGAGGCCGCCGTGATGGAGGATTACGCCGG |
| | | CTCTGATGGCAACATCGAACTGACCATCACCGGCATGACCTGTGCT |
| | | TCCTGCGTGCATAATATCGAGAGCAAACTGACGCGGACCAATGGA |
| | | ATCACCTATGCCAGCGTCGCCCTGGCAACCTCTAAGGCTCTGGTGA |
| | | AGTTTGACCCCGAAATCATTGGACCTAGAGACATCATCAAAATTAT |
| | | TGAAGAGATTGGTTTCCACGCCAGCTTAGCTCAGCGGAATCCCAAC |
| | | GCACACCATCTTGACCATAAGATGGAAATCAAACAGTGGAAGAAG |
| | | AGCTTCTTGTGTAGCCTGGTCTTCGGGATACCCGTCATGGCACTGA |
| | | TGATTTACATGCTCATTCCGTCCAACGAGCCCCACCAGTCCATGGT |
| | | GCTGGACCACAACATCATCCCTGGGCTGTCTATCCTCAACCTGATC |
| | | TTCTTTATTCTGTGCACATTTGTCCAGCTCTTGGGAGGCTGGTATTT |
| | | CTATGTACAGGCCTACAAATCCCTGAGGCATCGAAGCGCCAACAT |
| | | GGACGTTTTAATTGTGCTGGCTACATCCATTGCCTACGTGTACTCC |
| | | CTGGTGATCTTAGTGGTTGCTGTGGCTGAAAAGGCTGAGAGAAGC |
| | | CCTGTCACCTTCTTCGACACCCCCCCCATGCTCTTCGTGTTTATTGC |
| | | ACTGGGTCGCTGGCTGGAGCATTTAGCCAAGTCCAAGACATCCGA |
| | | GGCTCTGGCCAAGCTGATGTCTCTACAGGCCACGGAGGCCACCGT |
| | | GGTGACTCTGGGTGAAGACAATTTGATAATACGCGAGGAACAGGT |
| | | GCCTATGGAGCTTGTGCAGCGCGGAGATATCGTCAAGGTGGTGCC |
| | | CGGCGGCAAGTTCCCAGTGGATGGAAAAGTGTTAGAGGGCAATAC |
| | | CATGGCCGATGAATCCCTCATCACTGGCGAGGCAATGCCGGTGAC |
| | | CAAGAAACCTGGAAGTACTGTCATTGCAGGCAGCATCAATGCCCA |
| | | TGGGAGCGTCCTGATCAAGGCCACCCACGTGGGCAATGATACAAC |
| | | TCTCGCCCAGATCGTCAAGCTAGTGGAAGAGGCTCAGATGTCTAA |
| | | AGCTCCAATCCAGCAACTGGCTGACCGCTTCAGCGGCTACTTTGTC |
| | | CCCTTTATCATCATCATGAGCACCCTCACCCTGGTGGTGTGGATAG |
| | | TCATAGGCTTCATTGACTTTGGCGTGGTGCAGAGATACTTCCCCAA |
| | | CCCTAACAAACATATCTCCCAAACCGAGGTCATTATCAGATTCGCC |
| | | TTTCAGACATCTATCACTGTGCTGTGCATAGCCTGCCCTTGCTCTCT |
| | | GGGCCTGGCCACCCCCACCGCCGTGATGGTTGGAACCGGGGTCGC |
| | | TGCACAAAATGGGATCCTGATAAAAGGGGGCAAACCCCTGGAAAT |
| | | GGCACATAAGATTAAAACAGTTATGTTTGATAAGACAGGCACCAT |
| | | TACTCACGGTGTCCCTCGAGTCATGAGAGTGCTGCTGCTGGGGGAC |
| | | GTGGCTACACTGCCACTGCGCAAGGTGCTGGCTGTGGTGGGCACA |
| | | GCGGAGGCCTCCTCAGAACACCCATTAGGCGTAGCTGTCACAAAA |
| | | TACTGTAAGGAGGAGCTGGGGACAGAGACCCTCGGCTATTGTACA |
| | | GACTTCCAGGCCGTGCCTGGCTGCGGCATCGGCTGTAAGGTCAGC |
| | | AATGTGGAGGGCATCCTGGCACACAGCGAGAGACCCCTCTCTGCC |
| | | CCAGCCTCCCACCTGAATGAAGCTGGGTCTCTGCCTGCTGAGAAGG |
| | | ACGCCGTCCCTCAGACCTTCTCCGTGTTGATTGGGAACAGAGAGTG |
| | | GCTTAGAAGGAATGGCCTGACCATCAGCTCTGACGTCTCGGATGCC |
| | | ATGACAGATCACGAGATGAAAGGCCAGACAGCCATACTGGTTGCC |
| | | ATCGACGGCGTGCTCTGTGGCATGATTGCCATCGCTGATGCTGTTA |
| | | AACAGGAAGCTGCGCTGGCAGTCCACACCCTCCAGTCCATGGGAG |
| | | TGGACGTGGTCCTGATTACTGGAGACAACAGGAAAACCGCTCGGG |
| | | CCATCGCTACCCAAGTGGGGATAAATAAGGTTTTTGCAGAAGTCTT |
| | | ACCCTCCCACAAAGTAGCCAAAGTGCAGGAGCTCCAGAACAAGGG |
| | | TAAAAAGGTGGCAATGGTGGGAGATGGTGTGAATGACAGCCCAGC |
| | | TCTCGCGCAGGCTGACATGGGCGTAGCCATAGGTACTGGAACAGA |
| | | TGTCGCGATTGAAGCCGCTGACGTAGTCCTCATCCGAAACGATTTG |
| | | CTGGATGTGGTGGCCAGCATCCATCTGTCCAAGCGTACCGTGCGCC |
| | | GGATCCGCATCAACCTGGTCTTGGCCCTCATCTATAATCTCGTGGG |
| | | CATCCCCATCGCTGCTGGGGTCTTCATGCCCATCGGCATTGTGCTG |
| | | CAGCCCTGGATGGGAAGCGCTGCCATGGCTGCTTCTTCTGTCTCCG |
| | | TTGTGCTGTCCAGCCTACAGCTGAAGTGCTATAAAAAGCCAGACCT |
| | | CGAGCGCTACGAAGCCCAGGCGCATGGCCATATGAAGCCCCTCAC |
| | | GGCTTCCCAGGTCTCGGTTCACATTGGGATGGATGACAGATGGCG |
| | | GGACTCCCCACGCGCCACACCCTGGGACCAGGTATCCTATGTGAG |
| | | CCAGGTGAGCCTCAGCTCCCTGACATCAGATAAACCGTCTAGGCA |
| | | CTCAGCTGCCGCAGATGATGACGGTGACAAATGGAGCCTCCTCCT |
| | | GAACGGCCGCGACGAGGAGCAGTACATCTAAGAATTCGATATCAA |
| | | GCTTATCGATAATCAACCTCTGGATTACAAAATTTGTGAAAGATTG |
| | | ACTGGTATTCTTAACTATGTTGCTCCTTTTACGCTATGTGGATACGC |
| | | TGCTTTAATGCCTTTGTATCATGCTATTGCTTCCCGTATGGCTTTCA |
| | | TTTTCTCCTCCTTGTATAAATCCTGGTTAGTTCTTGCCACGGCGGAA |
| | | CTCATCGCCGCCTGCCTTGCCCGCTGCTGGACAGGGGCTCGGCTGT |
| | | TGGGCACTGACAATTCCGTGGTGTTCACCTGCAGGAATAAAAGAT |
| | | CTTTATTTTCATTAGATCTGTGTGTTGGTTTTTTGTGTGTCTAGAGT |
| | | AGATAAGTAGCATGGCGGGTTAATCATTAACTACAAGGAACCCCT |
| | | AGTGATGGAGTTGGCCACTCCCTCTCTGCGCGCTCGCTCGCTCACT |
| | | GAGGCCGGGCGACCAAAGGTCGCCCGACGCCCGGGCTTTGCCCGG |
| | | GCGGCCTCAGTGAGCGAGCGAGCGCGCAGAGAGGGAGTGGCCAA |
| 83 | A30 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTAATAATTACCAGCGCGGGCCAAAT |
| | | AAATAATCCGCGAGGGGCAGCTGGACTTTGGACTCCGCGCGCTGG |
| | | TAATTATTAACCTCGCGAATATTGATTCGAGGCCGCGATTGCCGCA |
| | | ATCGCGAGGGGCAGGTGACCTTTGCCCAGCGCGCGTTCGCCCCGC |
| | | CCCGGACGGTATCGATAAGCTTAGGAGCTTGGGCTGCAGGTCGAG |
| | | GGCACTGGGAGGATGTTGAGTAAGATGGAAAACTACTGATGACCC |
| | | TTGCAGAGACAGAGTATTAGGACATGTTTGAACAGGGGCCGGGCG |
| | | ATCAGCAGGTAGCTCTAGAGGATCCCCGTCTGTCTGCACATTTCGT |
| | | AGAGCGAGTGTTCCGATACTCTAATCTCCCTAGGCAAGGTTCATAT |
| | | TTGTGTAGGTTACTTATTCTCCTTTTGTTGACTAAGTCAATAATCAG |
| | | AATCAGCAGGTTTGGAGTCAGCTTGGCAGGGATCAGCAGCCTGGG |
| | | TTGGAAGGAGGGGGTATAAAAGCCCCTTCACCAGGAGAAGCCGTC |
| | | ACACAGATCCACAAGCTCGAGCCGAGTGGTCGTGCCTCCATAGAA |
| | | GCGGCCGCAAGATGGCGGCGCCTGAGCAGGAGAGACAGATCACA |
| | | GCCAGAGAAGGGGCCAGTCGGAAAATCTTATCTAAGCTTTCTTTGC |
| | | CTACCCGTGCCTGGGAACCAGCAATGAAGAAGAGTTTTGCTTTTGA |
| | | CAATGTTGGCTATGAAGGTGGTCTGGATGGCCTGGGCCCCAGCAG |
| | | CCAGGTGGCGACCACCTTAATCGCCATCGCAGGCATGACCTGCGC |
| | | ATCTTGTGTGCATAGCATTGAAGGCATGATTAGCCAGCTGGAGGG |
| | | CGTGCAGCAGATCTCAGTCTCCCTGGCTGAGGGGACCGCCACTGTC |
| | | TTGTACAATCCGTCTGTTATTTCCCCGGAGGAGCTGAGAGCTGCCA |
| | | TTGAGGACATGGGATTTGAAGCATCGGTGGTGTCTGAGTCATGCTC |
| | | CACCAATCCCCTGGGAAATCACTCAGCTGGCAATTCAATGGTTCAG |
| | | ACCACCGATGGGACCCCAACCAGCGTGCAGGAAGTGGCACCCCAT |
| | | ACCGGCAGACTGCCTGCCAATCATGCTCCTGATATCCTCGCCAAGA |
| | | GCCCCCAGAGCACCCGCGCGGTGGCACCCCAGAAGTGTTTCCTGC |
| | | AGATCAAGGGCATGACCTGTGCCAGCTGTGTTAGCAATATCGAAA |
| | | GAAACCTCCAAAAAGAGGCTGGAGTTCTGAGTGTTCTAGTGGCTTT |
| | | AATGGCCGGGAAAGCCGAGATCAAGTATGATCCCGAGGTGATTCA |
| | | GCCATTGGAGATTGCCCAGTTCATCCAGGATCTGGGCTTCGAGGCC |
| | | GCCGTGATGGAGGATTACGCCGGCTCTGATGGCAACATCGAACTG |
| | | ACCATCACCGGCATGACCTGTGCTTCCTGCGTGCATAATATCGAGA |
| | | GCAAACTGACGCGGACCAATGGAATCACCTATGCCAGCGTCGCCC |
| | | TGGCAACCTCTAAGGCTCTGGTGAAGTTTGACCCCGAAATCATTGG |
| | | ACCTAGAGACATCATCAAAATTATTGAAGAGATTGGTTTCCACGCC |
| | | AGCTTAGCTCAGCGGAATCCCAACGCACACCATCTTGACCATAAG |
| | | ATGGAAATCAAACAGTGGAAGAAGAGCTTCTTGTGTAGCCTGGTC |
| | | TTCGGGATACCCGTCATGGCACTGATGATTTACATGCTCATTCCGT |
| | | CCAACGAGCCCCACCAGTCCATGGTGCTGGACCACAACATCATCC |
| | | CTGGGCTGTCTATCCTCAACCTGATCTTCTTTATTCTGTGCACATTT |
| | | GTCCAGCTCTTGGGAGGCTGGTATTTCTATGTACAGGCCTACAAAT |
| | | CCCTGAGGCATCGAAGCGCCAACATGGACGTTTTAATTGTGCTGGC |
| | | TACATCCATTGCCTACGTGTACTCCCTGGTGATCTTAGTGGTTGCTG |
| | | TGGCTGAAAAGGCTGAGAGAAGCCCTGTCACCTTCTTCGACACCCC |
| | | CCCCATGCTCTTCGTGTTTATTGCACTGGGTCGCTGGCTGGAGCAT |
| | | TTAGCCAAGTCCAAGACATCCGAGGCTCTGGCCAAGCTGATGTCTC |
| | | TACAGGCCACGGAGGCCACCGTGGTGACTCTGGGTGAAGACAATT |
| | | TGATAATACGCGAGGAACAGGTGCCTATGGAGCTTGTGCAGCGCG |
| | | GAGATATCGTCAAGGTGGTGCCCGGCGGCAAGTTCCCAGTGGATG |
| | | GAAAAGTGTTAGAGGGCAATACCATGGCCGATGAATCCCTCATCA |
| | | CTGGCGAGGCAATGCCGGTGACCAAGAAACCTGGAAGTACTGTCA |
| | | TTGCAGGCAGCATCAATGCCCATGGGAGCGTCCTGATCAAGGCCA |
| | | CCCACGTGGGCAATGATACAACTCTCGCCCAGATCGTCAAGCTAGT |
| | | GGAAGAGGCTCAGATGTCTAAAGCTCCAATCCAGCAACTGGCTGA |
| | | CCGCTTCAGCGGCTACTTTGTCCCCTTTATCATCATCATGAGCACCC |
| | | TCACCCTGGTGGTGTGGATAGTCATAGGCTTCATTGACTTTGGCGT |
| | | GGTGCAGAGATACTTCCCCAACCCTAACAAACATATCTCCCAAACC |
| | | GAGGTCATTATCAGATTCGCCTTTCAGACATCTATCACTGTGCTGT |
| | | GCATAGCCTGCCCTTGCTCTCTGGGCCTGGCCACCCCCACCGCCGT |
| | | GATGGTTGGAACCGGGGTCGCTGCACAAAATGGGATCCTGATAAA |
| | | AGGGGGCAAACCCCTGGAAATGGCACATAAGATTAAAACAGTTAT |
| | | GTTTGATAAGACAGGCACCATTACTCACGGTGTCCCTCGAGTCATG |
| | | AGAGTGCTGCTGCTGGGGGACGTGGCTACACTGCCACTGCGCAAG |
| | | GTGCTGGCTGTGGTGGGCACAGCGGAGGCCTCCTCAGAACACCCA |
| | | TTAGGCGTAGCTGTCACAAAATACTGTAAGGAGGAGCTGGGGACA |
| | | GAGACCCTCGGCTATTGTACAGACTTCCAGGCCGTGCCTGGCTGCG |
| | | GCATCGGCTGTAAGGTCAGCAATGTGGAGGGCATCCTGGCACACA |
| | | GCGAGAGACCCCTCTCTGCCCCAGCCTCCCACCTGAATGAAGCTGG |
| | | GTCTCTGCCTGCTGAGAAGGACGCCGTCCCTCAGACCTTCTCCGTG |
| | | TTGATTGGGAACAGAGAGTGGCTTAGAAGGAATGGCCTGACCATC |
| | | AGCTCTGACGTCTCGGATGCCATGACAGATCACGAGATGAAAGGC |
| | | CAGACAGCCATACTGGTTGCCATCGACGGCGTGCTCTGTGGCATGA |
| | | TTGCCATCGCTGATGCTGTTAAACAGGAAGCTGCGCTGGCAGTCCA |
| | | CACCCTCCAGTCCATGGGAGTGGACGTGGTCCTGATTACTGGAGAC |
| | | AACAGGAAAACCGCTCGGGCCATCGCTACCCAAGTGGGGATAAAT |
| | | AAGGTTTTTGCAGAAGTCTTACCCTCCCACAAAGTAGCCAAAGTGC |
| | | AGGAGCTCCAGAACAAGGGTAAAAAGGTGGCAATGGTGGGAGAT |
| | | GGTGTGAATGACAGCCCAGCTCTCGCGCAGGCTGACATGGGCGTA |
| | | GCCATAGGTACTGGAACAGATGTCGCGATTGAAGCCGCTGACGTA |
| | | GTCCTCATCCGAAACGATTTGCTGGATGTGGTGGCCAGCATCCATC |
| | | TGTCCAAGCGTACCGTGCGCCGGATCCGCATCAACCTGGTCTTGGC |
| | | CCTCATCTATAATCTCGTGGGCATCCCCATCGCTGCTGGGGTCTTC |
| | | ATGCCCATCGGCATTGTGCTGCAGCCCTGGATGGGAAGCGCTGCC |
| | | ATGGCTGCTTCTTCTGTCTCCGTTGTGCTGTCCAGCCTACAGCTGAA |
| | | GTGCTATAAAAAGCCAGACCTCGAGCGCTACGAAGCCCAGGCGCA |
| | | TGGCCATATGAAGCCCCTCACGGCTTCCCAGGTCTCGGTTCACATT |
| | | GGGATGGATGACAGATGGCGGGACTCCCCACGCGCCACACCCTGG |
| | | GACCAGGTATCCTATGTGAGCCAGGTGAGCCTCAGCTCCCTGACAT |
| | | CAGATAAACCGTCTAGGCACTCAGCTGCCGCAGATGATGACGGTG |
| | | ACAAATGGAGCCTCCTCCTGAACGGCCGCGACGAGGAGCAGTACA |
| | | TCTAAGAATTCGATATCAAGCTTATCGATAATCAACCTCTGGATTA |
| | | CAAAATTTGTGAAAGATTGACTGGTATTCTTAACTATGTTGCTCCT |
| | | TTTACGCTATGTGGATACGCTGCTTTAATGCCTTTGTATCATGCTAT |
| | | TGCTTCCCGTATGGCTTTCATTTTCTCCTCCTTGTATAAATCCTGGT |
| | | TAGTTCTTGCCACGGCGGAACTCATCGCCGCCTGCCTTGCCCGCTG |
| | | CTGGACAGGGGCTCGGCTGTTGGGCACTGACAATTCCGTGGTGTTC |
| | | ACCTGCAGGAATAAAAGATCTTTATTTTCATTAGATCTGTGTGTTG |
| | | GTTTTTTGTGTGTCTAGAGTAGATAAGTAGCATGGCGGGTTAATCA |
| | | TTAACTACAAGGAACCCCTAGTGATGGAGTTGGCCACTCCCTCTCT |
| | | GCGCGCTCGCTCGCTCACTGAGGCCGGGCGACCAAAGGTCGCCCG |
| | | ACGCCCGGGCTTTGCCCGGGCGGCCTCAGTGAGCGAGCGAGCGCG |
| | | CAGAGAGGGAGTGGCCAA |
| 84 | A31 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTAATAATTACCAGCGCGGGCCAAAT |
| | | AAATAATCCGCGAGGGGCAGCTGGACTTTGGACTCCGCGCGCTGG |
| | | TAATTATTAACCTCGCGAATATTGATTCGAGGCCGCGATTGCCGCA |
| | | ATCGCGAGGGGCAGGTGACCTTTGCCCAGCGCGCGTTCGCCCCGC |
| | | CCCGGACGGTATCGATAAGCTTAGGAGCTTGGGCTGCAGGTCGAG |
| | | GGCACTGGGAGGATGTTGAGTAAGATGGAAAACTACTGATGACCC |
| | | TTGCAGAGACAGAGTATTAGGACATGTTTGAACAGGGGCCGGGCG |
| | | ATCAGCAGGTAGCTCTAGAGGATCCCCGTCTGTCTGCACATTTCGT |
| | | AGAGCGAGTGTTCCGATACTCTAATCTCCCTAGGCAAGGTTCATAT |
| | | TTGTGTAGGTTACTTATTCTCCTTTTGTTGACTAAGTCAATAATCAG |
| | | AATCAGCAGGTTTGGAGTCAGCTTGGCAGGGATCAGCAGCCTGGG |
| | | TTGGAAGGAGGGGGTATAAAAGCCCCTTCACCAGGAGAAGCCGTC |
| | | ACACAGATCCACAAGCTCGAGCCGAGTGGTCGTGCCTCCATAGAA |
| | | GCGGCCGCAAGATGGCGGCGCCTGAGCAGGAGAGACAGATCACA |
| | | GCCAGAGAAGGGGCCAGTCGGAAAATCTTATCTAAGCTTTCTTTGC |
| | | CTACCCGTGCCTGGGAACCAGCAATGAAGAAGAGTTTTGCTTTTGA |
| | | CAATGTTGGCTATGAAGGTGGTCTGGATGGCCTGGGCCCTTCTTCT |
| | | CAGGTGGCCCCTGATGGAGCCGAAGGGAGTGGGACAGATCACAGG |
| | | TCTTCCAGTTCTCATTCCCCTGGCTCCCCACCGAGAAACCAGGTCC |
| | | AGGGCACATGCAGTAAGTGCTTCTTACAGATCAAAGGCATGACCT |
| | | GTGCATCCTGTGTGTCTAACATAGAAAGGAATCTGCAGAAAGAAG |
| | | CTGGTGTTCTCTCCGTGTTGGTTGCCTTGATGGCAGGAAAGGCAGA |
| | | GATCAAGTATGACCCAGAGGTCATCCAGCCCCTCGAGATAGCTCA |
| | | GTTCATCCAGGACCTGGGTTTTGAGGCAGCAGTCATGGAGGACTA |
| | | CGCAGGCTCCGATGGCAACATTGAGCTGACAATCACAGGGATGAC |
| | | CTGCGCGTCCTGTGTCCACAACATAGAGTCCAAACTCACGAGGAC |
| | | AAATGGCATCACTTATGCCTCCGTTGCCCTTGCCACCAGCAAAGCC |
| | | CTTGTTAAGTTTGACCCGGAAATTATCGGTCCACGGGATATTATCA |
| | | AAATTATTGAGGAAATTGGCTTTCATGCTTCCCTGGCCCAGAGAAA |
| | | CCCCAACGCTCATCACTTGGACCACAAGATGGAAATAAAGCAGTG |
| | | GAAGAAGTCTTTTCTTGTGAGTCTGTTTTTCTGTATTCCTGTAATGG |
| | | GGCTGATGATATATATGATGGTTATGGACCACCACTTTGCAACTCT |
| | | TCACCATAATCAAAACATGAGTAAAGAAGAAATGATCAACCTTCA |
| | | TTCTTCTATGTTCCTGGAGCGCCAGATTCTTCCAGGATTGTCTGTTA |
| | | TGAATTTGCTGTCCTTTTTATTGTGTGTACCTGTACAGTTTTTCGGA |
| | | GGCTGGTACTTCTACATTCAGGCTTATAAAGCACTGAAGCATAAGA |
| | | CAGCAAATATGGACGTACTGATTGTGCTGGCAACCACCATTGCATT |
| | | TGCCTACTCTTTGATTATTCTTCTAGTTGCAATGTATGAGAGAGCC |
| | | AAAGTGAACCCTATTACTTTCTTTGACACACCCCCTATGCTGTTTGT |
| | | GTTTATTGCACTAGGCCGATGGCTGGAACATATAGCAAAGGGCAA |
| | | AACATCAGAGGCTCTTGCAAAGTTAATTTCACTACAAGCTACAGA |
| | | AGCAACTATTGTAACTCTTGATTCTGATAATATCCTCCTCAGTGAA |
| | | GAACAAGTGGATGTGGAACTTGTACAACGTGGAGATATCATTAAA |
| | | GTAGTTCCAGGAGGCAAATTTCCAGTGGATGGTCGTGTTATTGAAG |
| | | GACATTCTATGGTAGATGAGTCCCTCATCACAGGGGAGGCAATGC |
| | | CTGTGGCTAAGAAACCTGGCAGCACAGTGATTGCTGGTTCCATTAA |
| | | CCAGAACGGGTCACTGCTTATCTGCGCAACACATGTTGGAGCAGA |
| | | CACAACCCTTTCTCAAATTGTCAAACTTGTGGAAGAGGCACAAAC |
| | | ATCAAAGGCTCCTATCCAGCAGTTTGCAGACAAACTCAGTGGCTAT |
| | | TTTGTTCCTTTTATTGTTTTTGTTTCCATTGCCACCCTCTTGGTATGG |
| | | ATTGTAATTGGATTTCTGAATTTTGAAATTGTGGAAACCTACTTTCC |
| | | TGGCTACAATAGAAGTATCTCCCGAACAGAAACGATAATACGATT |
| | | TGCTTTCCAAGCCTCTATCACAGTTCTGTGTATTGCATGTCCCTGTT |
| | | CACTGGGACTGGCCACTCCAACTGCTGTGATGGTGGGTACAGGAG |
| | | TAGGTGCTCAAAATGGCATACTAATAAAAGGTGGAGAGCCATTGG |
| | | AGATGGCTCATAAGGTAAAGGTAGTGGTATTTGATAAGACTGGAA |
| | | CCATTACTCACGGAACCCCAGTGGTGAATCAAGTAAAGGTTCTAA |
| | | CTGAAAGTAACAGAATATCACACCATAAAATCTTGGCCATTGTGG |
| | | GAACTGCTGAAAGTAACAGTGAACACCCTCTAGGAACAGCCATAA |
| | | CCAAATATTGCAAACAGGAGCTGGACACTGAAACCTTGGGTACCT |
| | | GCATAGATTTCCAGGTTGTGCCAGGCTGTGGTATTAGCTGTAAAGT |
| | | CACCAATATTGAAGGCTTGCTACATAAGAATAACTGGAATATAGA |
| | | GGACAATAATATTAAAAATGCATCCCTGGTTCAAATTGATGCCAGT |
| | | AATGAACAGTCATCAACTTCGTCTTCCATGATTATTGATGCCCAGA |
| | | TCTCAAATGCTCTTAATGCTCAGCAGTATAAAGTCCTCATTGGTAA |
| | | CCGGGAGTGGATGATTAGAAATGGTCTTGTCATTAATAACGATGTA |
| | | AATGATTTCATGACTGAACATGAGAGAAAAGGTCGGACTGCTGTA |
| | | TTAGTAGCAGTTGATGATGAGCTGTGTGGCTTGATAGCCATTGCAG |
| | | ACACAGTGAAGCCTGAAGCAGAACTGGCTATCCATATTCTGAAAT |
| | | CTATGGGCTTAGAAGTAGTTCTGATGACTGGAGACAACAGTAAAA |
| | | CAGCTAGATCTATTGCTTCTCAGGTTGGCATTACTAAGGTGTTTGC |
| | | TGAAGTTCTACCTTCTCACAAGGTTGCTAAAGTGAAGCAACTTCAA |
| | | GAGGAGGGGAAACGGGTAGCAATGGTGGGAGATGGAATCAATGA |
| | | CTCCCCAGCTCTGGCAATGGCTAATGTGGGAATTGCTATTGGCACA |
| | | GGCACAGATGTAGCCATTGAAGCAGCTGATGTGGTTTTGATAAGG |
| | | AATGATCTTCTGGATGTAGTGGCAAGTATTGACTTATCAAGAAAGA |
| | | CAGTCAAGAGGATTCGGATAAATTTTGTCTTTGCTCTAATTTATAA |
| | | TCTGGTTGGAATTCCCATAGCTGCTGGAGTTTTTATGCCCATTGGTT |
| | | TGGTTTTGCAGCCCTGGATGGGATCTGCAGCAATGGCTGCTTCATC |
| | | TGTTTCTGTAGTACTTTCTTCTCTCCAGCTCAAGTGCTATAAGAAGC |
| | | CTGACCTGGAGAGGTATGAGGCACAGGCGCATGGCCACATGAAGC |
| | | CCCTGACGGCATCCCAGGTCAGTGTGCACATAGGCATGGATGACA |
| | | GGTGGCGGGACTCCCCCAGGGCCACACCATGGGACCAGGTCAGCT |
| | | ATGTCAGCCAGGTGTCGCTGTCCTCCCTGACGTCCGACAAGCCATC |
| | | TCGGCACAGCGCTGCAGCAGACGATGATGGGGACAAGTGGTCTCT |
| | | GCTCCTGAATGGCAGGGATGAGGAGCAGTACATCTGAGAATTCGA |
| | | TATCAAGCTTATCGATAATCAACCTCTGGATTACAAAATTTGTGAA |
| | | AGATTGACTGGTATTCTTAACTATGTTGCTCCTTTTACGCTATGTGG |
| | | ATACGCTGCTTTAATGCCTTTGTATCATGCTATTGCTTCCCGTATGG |
| | | CTTTCATTTTCTCCTCCTTGTATAAATCCTGGTTAGTTCTTGCCACG |
| | | GCGGAACTCATCGCCGCCTGCCTTGCCCGCTGCTGGACAGGGGCTC |
| | | GGCTGTTGGGCACTGACAATTCCGTGGTGTTCACCTGCAGGAATAA |
| | | AAGATCTTTATTTTCATTAGATCTGTGTGTTGGTTTTTTGTGTGTCT |
| | | AGAGTAGATAAGTAGCATGGCGGGTTAATCATTAACTACAAGGAA |
| | | CCCCTAGTGATGGAGTTGGCCACTCCCTCTCTGCGCGCTCGCTCGC |
| | | TCACTGAGGCCGGGCGACCAAAGGTCGCCCGACGCCCGGGCTTTG |
| | | CCCGGGCGGCCTCAGTGAGCGAGCGAGCGCGCAGAGAGGGAGTG |
| | | GCCAA |
| 85 | A32 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTAATAATTACCAGCGCGGGCCAAAT |
| | | AAATAATCCGCGAGGGGCAGCTGGACTTTGGACTCCGCGCGCTGG |
| | | TAATTATTAACCTCGCGAATATTGATTCGAGGCCGCGATTGCCGCA |
| | | ATCGCGAGGGGCAGGTGACCTTTGCCCAGCGCGCGTTCGCCCCGC |
| | | CCCGGACGGTATCGATAAGCTTAGGAGCTTGGGCTGCAGGTCGAG |
| | | GGCACTGGGAGGATGTTGAGTAAGATGGAAAACTACTGATGACCC |
| | | TTGCAGAGACAGAGTATTAGGACATGTTTGAACAGGGGCCGGGCG |
| | | ATCAGCAGGTAGCTCTAGAGGATCCCCGTCTGTCTGCACATTTCGT |
| | | AGAGCGAGTGTTCCGATACTCTAATCTCCCTAGGCAAGGTTCATAT |
| | | TTGTGTAGGTTACTTATTCTCCTTTTGTTGACTAAGTCAATAATCAG |
| | | AATCAGCAGGTTTGGAGTCAGCTTGGCAGGGATCAGCAGCCTGGG |
| | | TTGGAAGGAGGGGGTATAAAAGCCCCTTCACCAGGAGAAGCCGTC |
| | | ACACAGATCCACAAGCTCGAGCCGAGTGGTCGTGCCTCCATAGAA |
| | | GCGGCCGCAAGATGGCGGCGCCTGAGCAGGAGAGACAGATCACA |
| | | GCCAGAGAAGGGGCCAGTCGGAAAATCTTATCTAAGCTTTCTTTGC |
| | | CTACCCGTGCCTGGGAACCAGCAATGAAGAAGAGTTTTGCTTTTGA |
| | | CAATGTTGGCTATGAAGGTGGTCTGGATGGCCTGGGCCCTAGCAG |
| | | CCAGGTGGCTACCACCCTGATTGCCATTGCCGGCATGACCTGCGCC |
| | | AGCTGCGTGCATAGCATTGAGGGCATGATTAGCCAGCTGGAGGGC |
| | | GTGCAGCAGATTTCCGTGAGCCTGGCTGAGGGCACCGCTACCGTG |
| | | CTGTATAATCCCTCCGTGATTAGCCCCGAAGAACTGCGCGCTGCCA |
| | | TTGAAGATATGGGCTTTGAAGCCTCCGTGGTGTCCGAAAGCTGCAG |
| | | CACCAATCCCCTGGGCAATCATTCCGCCGGCAATAGCATGGTGCA |
| | | GACCACCGACGGCACCCCCACCTCCGTGCAGGAAGTGGCTCCCCA |
| | | TACCGGCCGCCTGCCCGCCAATCATGCCCCTGATATTCTGGCTAAA |
| | | AGCCCCCAGAGCACCCGCGCCGTGGCTCCTCAGAAATGCTTTCTGC |
| | | AGATTAAGGGCATGACCTGCGCTAGCTGCGTGAGCAATATTGAAC |
| | | GCAATCTGCAGAAAGAAGCCGGCGTGCTGTCCGTGCTGGTGGCCC |
| | | TGATGGCCGGCAAAGCTGAAATCAAATATGATCCCGAAGTGATTC |
| | | AGCCCCTGGAAATTGCCCAGTTCATTCAGGATCTGGGCTTTGAGGC |
| | | TGCCGTGATGGAAGATTATGCCGGCTCCGACGGCAATATTGAACT |
| | | GACCATTACCGGCATGACCTGCGCCAGCTGCGTGCATAATATTGAA |
| | | AGCAAACTGACCCGCACCAACGGCATTACCTATGCTTCCGTGGCCC |
| | | TGGCTACCAGCAAAGCCCTGGTGAAATTTGATCCTGAAATTATCGG |
| | | CCCCCGCGATATTATCAAAATTATTGAAGAAATCGGCTTTCACGCC |
| | | AGCCTGGCTCAGCGCAATCCCAATGCCCATCATCTGGATCATAAGA |
| | | TGGAAATCAAACAGTGGAAGAAAAGCTTTCTGTGCAGCCTGGTGT |
| | | TCGGCATTCCTGTGATGGCCCTGATGATCTATATGCTGATTCCCAG |
| | | CAATGAACCCCATCAGAGCATGGTGCTGGATCATAATATTATTCCC |
| | | GGCCTGAGCATTCTGAATCTGATTTTCTTTATCCTGTGCACCTTTGT |
| | | GCAGCTGCTGGGCGGCTGGTATTTCTATGTGCAGGCTTATAAAAGC |
| | | CTGCGCCACCGCTCCGCCAATATGGATGTGCTGATTGTGCTGGCCA |
| | | CCAGCATTGCTTATGTCTATAGCCTGGTCATTCTGGTGGTGGCCGT |
| | | GGCCGAGAAAGCTGAACGCAGCCCCGTGACCTTCTTTGATACCCCT |
| | | CCCATGCTGTTCGTGTTTATCGCTCTGGGCCGCTGGCTGGAACATC |
| | | TGGCCAAAAGCAAAACCTCCGAAGCCCTGGCTAAACTGATGAGCC |
| | | TGCAGGCCACCGAAGCCACCGTGGTGACCCTGGGCGAAGATAATC |
| | | TGATTATCCGCGAAGAACAGGTGCCTATGGAACTGGTGCAGCGCG |
| | | GCGATATTGTGAAAGTGGTGCCCGGCGGCAAATTTCCCGTGGACG |
| | | GCAAAGTGCTGGAGGGCAATACCATGGCTGATGAATCCCTGATTA |
| | | CCGGCGAAGCTATGCCTGTGACCAAGAAACCCGGCAGCACCGTGA |
| | | TTGCCGGCAGCATTAATGCTCACGGCTCCGTGCTGATCAAGGCTAC |
| | | CCATGTCGGCAATGATACCACCCTGGCCCAGATTGTGAAACTGGTG |
| | | GAAGAAGCCCAGATGAGCAAAGCCCCTATTCAGCAGCTGGCTGAC |
| | | CGCTTTTCCGGCTATTTTGTGCCCTTTATCATTATTATGAGCACCCT |
| | | GACCCTGGTGGTGTGGATTGTGATCGGCTTCATTGATTTCGGCGTG |
| | | GTGCAGAGATATTTTCCTAATCCCAACAAACATATTAGCCAGACCG |
| | | AAGTGATTATCCGCTTTGCTTTTCAGACCAGCATTACCGTGCTGTG |
| | | CATTGCTTGCCCCTGCAGCCTGGGCCTGGCTACCCCCACCGCCGTC |
| | | ATGGTCGGCACCGGCGTGGCTGCCCAGAACGGCATTCTGATTAAG |
| | | GGCGGCAAACCTCTGGAAATGGCCCACAAAATCAAAACCGTGATG |
| | | TTTGATAAAACCGGCACCATTACCCACGGCGTGCCCCGCGTGATGC |
| | | GCGTGCTGCTGCTGGGCGATGTGGCTACCCTGCCCCTGCGCAAAGT |
| | | GCTGGCCGTGGTCGGCACCGCCGAAGCTAGCTCCGAACATCCCCT |
| | | GGGCGTGGCCGTGACCAAATATTGCAAAGAAGAACTGGGCACCGA |
| | | AACCCTGGGCTATTGCACCGATTTTCAGGCCGTCCCTGGCTGCGGC |
| | | ATCGGCTGCAAAGTGAGCAATGTGGAGGGCATTCTGGCCCATTCC |
| | | GAAAGACCCCTGTCCGCCCCTGCTAGCCATCTGAATGAAGCCGGC |
| | | AGCCTGCCTGCTGAGAAAGATGCCGTGCCCCAGACCTTTTCCGTGC |
| | | TGATCGGCAACCGCGAATGGCTGCGCCGCAACGGCCTGACCATTA |
| | | GCTCCGATGTCTCCGATGCCATGACCGATCATGAAATGAAAGGCC |
| | | AGACCGCCATTCTGGTGGCCATTGACGGCGTGCTGTGCGGCATGAT |
| | | TGCCATTGCCGATGCCGTGAAACAGGAGGCTGCCCTGGCTGTGCAT |
| | | ACCCTGCAGAGCATGGGCGTGGATGTGGTGCTGATTACCGGCGAT |
| | | AACCGCAAAACCGCTCGCGCCATTGCCACCCAGGTCGGCATTAAC |
| | | AAAGTGTTTGCTGAAGTGCTGCCCAGCCATAAAGTGGCTAAAGTG |
| | | CAGGAACTGCAGAACAAAGGCAAGAAAGTGGCTATGGTCGGCGA |
| | | CGGCGTGAATGATAGCCCTGCCCTGGCCCAGGCCGATATGGGCGT |
| | | GGCTATCGGCACCGGCACCGATGTGGCTATTGAGGCTGCCGATGT |
| | | GGTGCTGATCCGCAATGATCTGCTGGATGTGGTGGCTAGCATTCAT |
| | | CTGAGCAAACGCACCGTCCGCCGCATCCGCATTAATCTGGTGCTGG |
| | | CCCTGATCTATAATCTGGTCGGCATTCCCATCGCCGCCGGCGTGTT |
| | | TATGCCCATCGGCATTGTGCTGCAGCCCTGGATGGGCAGCGCCGCT |
| | | ATGGCCGCTAGCTCCGTGTCCGTGGTGCTGAGCAGCCTGCAGCTGA |
| | | AATGCTATAAGAAACCCGATCTGGAAAGATATGAAGCTCAGGCTC |
| | | ACGGCCATATGAAACCCCTGACCGCCAGCCAGGTGTCCGTGCATA |
| | | TCGGCATGGATGACCGCTGGCGCGATAGCCCCCGCGCCACCCCTTG |
| | | GGATCAGGTGAGCTATGTGAGCCAGGTGTCCCTGAGCTCCCTGACC |
| | | TCCGATAAACCCTCCCGCCATTCCGCCGCTGCCGATGATGACGGCG |
| | | ATAAGTGGAGCCTGCTGCTGAACGGCCGCGATGAAGAACAGTATA |
| | | TCTAAGTCCATAAAGTAGGAAACACTACATCATCCATAAAGTAGG |
| | | AAACACTACATCATCCATAAAGTAGGAAACACTACATCATCCATA |
| | | AAGTAGGAAACACTACAGAATTCGATATCAAGCTTATCGATAATC |
| | | AACCTCTGGATTACAAAATTTGTGAAAGATTGACTGGTATTCTTAA |
| | | CTATGTTGCTCCTTTTACGCTATGTGGATACGCTGCTTTAATGCCTT |
| | | TGTATCATGCTATTGCTTCCCGTATGGCTTTCATTTTCTCCTCCTTGT |
| | | ATAAATCCTGGTTAGTTCTTGCCACGGCGGAACTCATCGCCGCCTG |
| | | CCTTGCCCGCTGCTGGACAGGGGCTCGGCTGTTGGGCACTGACAAT |
| | | TCCGTGGTGTTCACCTGCAGGAATAAAAGATCTTTATTTTCATTAG |
| | | ATCTGTGTGTTGGTTTTTTGTGTGTCTAGAGTAGATAAGTAGCATG |
| | | GCGGGTTAATCATTAACTACAAGGAACCCCTAGTGATGGAGTTGG |
| | | CCACTCCCTCTCTGCGCGCTCGCTCGCTCACTGAGGCCGGGCGACC |
| | | AAAGGTCGCCCGACGCCCGGGCTTTGCCCGGGCGGCCTCAGTGAG |
| | | CGAGCGAGCGCGCAGAGAGGGAGTGGCCAA |
| 86 | A33 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCAAGATGGCGGCGCCT |
| | | GAGCAGGAGAGACAGATCACAGCCAGAGAAGGGGCCAGTCGGAA |
| | | AATCTTATCTAAGCTTTCTTTGCCTACCCGTGCCTGGGAACCAGCA |
| | | ATGAAGAAGAGTTTTGCTTTTGACAATGTTGGCTATGAAGGTGGTC |
| | | TGGATGGCCTGGGCCCCAGCAGCCAGCCCCAGAAGTGTTTCCTGC |
| | | AGATCAAGGGCATGACCTGTGCCAGCTGTGTTAGCAATATCGAAA |
| | | GAAACCTCCAAAAAGAGGCTGGAGTTCTGAGTGTTCTAGTGGCTTT |
| | | AATGGCCGGGAAAGCCGAGATCAAGTATGATCCCGAGGTGATTCA |
| | | GCCATTGGAGATTGCCCAGTTCATCCAGGATCTGGGCTTCGAGGCC |
| | | GCCGTGATGGAGGATTACGCCGGCTCTGATGGCAACATCGAACTG |
| | | ACCATCACCGGCATGACCTGTGCTTCCTGCGTGCATAATATCGAGA |
| | | GCAAACTGACGCGGACCAATGGAATCACCTATGCCAGCGTCGCCC |
| | | TGGCAACCTCTAAGGCTCTGGTGAAGTTTGACCCCGAAATCATTGG |
| | | ACCTAGAGACATCATCAAAATTATTGAAGAGATTGGTTTCCACGCC |
| | | AGCTTAGCTCAGCGGAATCCCAACGCACACCATCTTGACCATAAG |
| | | ATGGAAATCAAACAGTGGAAGAAGAGCTTCTTGTGTAGCCTGGTC |
| | | TTCGGGATACCCGTCATGGCACTGATGATTTACATGCTCATTCCGT |
| | | CCAACGAGCCCCACCAGTCCATGGTGCTGGACCACAACATCATCC |
| | | CTGGGCTGTCTATCCTCAACCTGATCTTCTTTATTCTGTGCACATTT |
| | | GTCCAGCTCTTGGGAGGCTGGTATTTCTATGTACAGGCCTACAAAT |
| | | CCCTGAGGCATCGAAGCGCCAACATGGACGTTTTAATTGTGCTGGC |
| | | TACATCCATTGCCTACGTGTACTCCCTGGTGATCTTAGTGGTTGCTG |
| | | TGGCTGAAAAGGCTGAGAGAAGCCCTGTCACCTTCTTCGACACCCC |
| | | CCCCATGCTCTTCGTGTTTATTGCACTGGGTCGCTGGCTGGAGCAT |
| | | TTAGCCAAGTCCAAGACATCCGAGGCTCTGGCCAAGCTGATGTCTC |
| | | TACAGGCCACGGAGGCCACCGTGGTGACTCTGGGTGAAGACAATT |
| | | TGATAATACGCGAGGAACAGGTGCCTATGGAGCTTGTGCAGCGCG |
| | | GAGATATCGTCAAGGTGGTGCCCGGCGGCAAGTTCCCAGTGGATG |
| | | GAAAAGTGTTAGAGGGCAATACCATGGCCGATGAATCCCTCATCA |
| | | CTGGCGAGGCAATGCCGGTGACCAAGAAACCTGGAAGTACTGTCA |
| | | TTGCAGGCAGCATCAATGCCCATGGGAGCGTCCTGATCAAGGCCA |
| | | CCCACGTGGGCAATGATACAACTCTCGCCCAGATCGTCAAGCTAGT |
| | | GGAAGAGGCTCAGATGTCTAAAGCTCCAATCCAGCAACTGGCTGA |
| | | CCGCTTCAGCGGCTACTTTGTCCCCTTTATCATCATCATGAGCACCC |
| | | TCACCCTGGTGGTGTGGATAGTCATAGGCTTCATTGACTTTGGCGT |
| | | GGTGCAGAGATACTTCCCCAACCCTAACAAACATATCTCCCAAACC |
| | | GAGGTCATTATCAGATTCGCCTTTCAGACATCTATCACTGTGCTGT |
| | | GCATAGCCTGCCCTTGCTCTCTGGGCCTGGCCACCCCCACCGCCGT |
| | | GATGGTTGGAACCGGGGTCGCTGCACAAAATGGGATCCTGATAAA |
| | | AGGGGGCAAACCCCTGGAAATGGCACATAAGATTAAAACAGTTAT |
| | | GTTTGATAAGACAGGCACCATTACTCACGGTGTCCCTCGAGTCATG |
| | | AGAGTGCTGCTGCTGGGGGACGTGGCTACACTGCCACTGCGCAAG |
| | | GTGCTGGCTGTGGTGGGCACAGCGGAGGCCTCCTCAGAACACCCA |
| | | TTAGGCGTAGCTGTCACAAAATACTGTAAGGAGGAGCTGGGGACA |
| | | GAGACCCTCGGCTATTGTACAGACTTCCAGGCCGTGCCTGGCTGCG |
| | | GCATCGGCTGTAAGGTCAGCAATGTGGAGGGCATCCTGGCACACA |
| | | GCGAGAGACCCCTCTCTGCCCCAGCCTCCCACCTGAATGAAGCTGG |
| | | GTCTCTGCCTGCTGAGAAGGACGCCGTCCCTCAGACCTTCTCCGTG |
| | | TTGATTGGGAACAGAGAGTGGCTTAGAAGGAATGGCCTGACCATC |
| | | AGCTCTGACGTCTCGGATGCCATGACAGATCACGAGATGAAAGGC |
| | | CAGACAGCCATACTGGTTGCCATCGACGGCGTGCTCTGTGGCATGA |
| | | TTGCCATCGCTGATGCTGTTAAACAGGAAGCTGCGCTGGCAGTCCA |
| | | CACCCTCCAGTCCATGGGAGTGGACGTGGTCCTGATTACTGGAGAC |
| | | AACAGGAAAACCGCTCGGGCCATCGCTACCCAAGTGGGGATAAAT |
| | | AAGGTTTTTGCAGAAGTCTTACCCTCCCACAAAGTAGCCAAAGTGC |
| | | AGGAGCTCCAGAACAAGGGTAAAAAGGTGGCAATGGTGGGAGAT |
| | | GGTGTGAATGACAGCCCAGCTCTCGCGCAGGCTGACATGGGCGTA |
| | | GCCATAGGTACTGGAACAGATGTCGCGATTGAAGCCGCTGACGTA |
| | | GTCCTCATCCGAAACGATTTGCTGGATGTGGTGGCCAGCATCCATC |
| | | TGTCCAAGCGTACCGTGCGCCGGATCCGCATCAACCTGGTCTTGGC |
| | | CCTCATCTATAATCTCGTGGGCATCCCCATCGCTGCTGGGGTCTTC |
| | | ATGCCCATCGGCATTGTGCTGCAGCCCTGGATGGGAAGCGCTGCC |
| | | ATGGCTGCTTCTTCTGTCTCCGTTGTGCTGTCCAGCCTACAGCTGAA |
| | | GTGCTATAAAAAGCCAGACCTCGAGCGCTACGAAGCCCAGGCGCA |
| | | TGGCCATATGAAGCCCCTCACGGCTTCCCAGGTCTCGGTTCACATT |
| | | GGGATGGATGACAGATGGCGGGACTCCCCACGCGCCACACCCTGG |
| | | GACCAGGTATCCTATGTGAGCCAGGTGAGCCTCAGCTCCCTGACAT |
| | | CAGATAAACCGTCTAGGCACTCAGCTGCCGCAGATGATGACGGTG |
| | | ACAAATGGAGCCTCCTCCTGAACGGCCGCGACGAGGAGCAGTACA |
| | | TCTAAGAATTCGATATCAAGCTTATCGATAATCAACCTCTGGATTA |
| | | CAAAATTTGTGAAAGATTGACTGGTATTCTTAACTATGTTGCTCCT |
| | | TTTACGCTATGTGGATACGCTGCTTTAATGCCTTTGTATCATGCTAT |
| | | TGCTTCCCGTATGGCTTTCATTTTCTCCTCCTTGTATAAATCCTGGT |
| | | TAGTTCTTGCCACGGCGGAACTCATCGCCGCCTGCCTTGCCCGCTG |
| | | CTGGACAGGGGCTCGGCTGTTGGGCACTGACAATTCCGTGGTGTTC |
| | | ACCTGCAGGAATAAAAGATCTTTATTTTCATTAGATCTGTGTGTTG |
| | | GTTTTTTGTGTGTCTAGAGTAGATAAGTAGCATGGCGGGTTAATCA |
| | | TTAACTACAAGGAACCCCTAGTGATGGAGTTGGCCACTCCCTCTCT |
| | | GCGCGCTCGCTCGCTCACTGAGGCCGGGCGACCAAAGGTCGCCCG |
| | | ACGCCCGGGCTTTGCCCGGGCGGCCTCAGTGAGCGAGCGAGCGCG |
| | | CAGAGAGGGAGTGGCCAA |
| 87 | A34 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCAAGATGGCGGCGCCT |
| | | GAGCAGGAGAGACAGATCACAGCCAGAGAAGGGGCCAGTCGGAA |
| | | AATCTTATCTAAGCTTTCTTTGCCTACCCGTGCCTGGGAACCAGCA |
| | | ATGAAGAAGAGTTTTGCTTTTGACAATGTTGGCTATGAAGGTGGTC |
| | | TGGATGGCCTGGGCCCCAGCAGCCAGGTGGCGACCACCTTAATCG |
| | | CCATCGCAGGCATGACCTGCGCATCTTGTGTGCATAGCATTGAAGG |
| | | CATGATTAGCCAGCTGGAGGGCGTGCAGCAGATCTCAGTCTCCCTG |
| | | GCTGAGGGGACCGCCACTGTCTTGTACAATCCGTCTGTTATTTCCC |
| | | CGGAGGAGCTGAGAGCTGCCATTGAGGACATGGGATTTGAAGCAT |
| | | CGGTGGTGTCTGAGTCATGCTCCACCAATCCCCTGGGAAATCACTC |
| | | AGCTGGCAATTCAATGGTTCAGACCACCGATGGGACCCCAACCAG |
| | | CGTGCAGGAAGTGGCACCCCATACCGGCAGACTGCCTGCCAATCA |
| | | TGCTCCTGATATCCTCGCCAAGAGCCCCCAGAGCACCCGCGCGGTG |
| | | GCACCCCAGAAGTGTTTCCTGCAGATCAAGGGCATGACCTGTGCC |
| | | AGCTGTGTTAGCAATATCGAAAGAAACCTCCAAAAAGAGGCTGGA |
| | | GTTCTGAGTGTTCTAGTGGCTTTAATGGCCGGGAAAGCCGAGATCA |
| | | AGTATGATCCCGAGGTGATTCAGCCATTGGAGATTGCCCAGTTCAT |
| | | CCAGGATCTGGGCTTCGAGGCCGCCGTGATGGAGGATTACGCCGG |
| | | CTCTGATGGCAACATCGAACTGACCATCACCGGCATGACCTGTGCT |
| | | TCCTGCGTGCATAATATCGAGAGCAAACTGACGCGGACCAATGGA |
| | | ATCACCTATGCCAGCGTCGCCCTGGCAACCTCTAAGGCTCTGGTGA |
| | | AGTTTGACCCCGAAATCATTGGACCTAGAGACATCATCAAAATTAT |
| | | TGAAGAGATTGGTTTCCACGCCAGCTTAGCTCAGCGGAATCCCAAC |
| | | GCACACCATCTTGACCATAAGATGGAAATCAAACAGTGGAAGAAG |
| | | AGCTTCTTGTGTAGCCTGGTCTTCGGGATACCCGTCATGGCACTGA |
| | | TGATTTACATGCTCATTCCGTCCAACGAGCCCCACCAGTCCATGGT |
| | | GCTGGACCACAACATCATCCCTGGGCTGTCTATCCTCAACCTGATC |
| | | TTCTTTATTCTGTGCACATTTGTCCAGCTCTTGGGAGGCTGGTATTT |
| | | CTATGTACAGGCCTACAAATCCCTGAGGCATCGAAGCGCCAACAT |
| | | GGACGTTTTAATTGTGCTGGCTACATCCATTGCCTACGTGTACTCC |
| | | CTGGTGATCTTAGTGGTTGCTGTGGCTGAAAAGGCTGAGAGAAGC |
| | | CCTGTCACCTTCTTCGACACCCCCCCCATGCTCTTCGTGTTTATTGC |
| | | ACTGGGTCGCTGGCTGGAGCATTTAGCCAAGTCCAAGACATCCGA |
| | | GGCTCTGGCCAAGCTGATGTCTCTACAGGCCACGGAGGCCACCGT |
| | | GGTGACTCTGGGTGAAGACAATTTGATAATACGCGAGGAACAGGT |
| | | GCCTATGGAGCTTGTGCAGCGCGGAGATATCGTCAAGGTGGTGCC |
| | | CGGCGGCAAGTTCCCAGTGGATGGAAAAGTGTTAGAGGGCAATAC |
| | | CATGGCCGATGAATCCCTCATCACTGGCGAGGCAATGCCGGTGAC |
| | | CAAGAAACCTGGAAGTACTGTCATTGCAGGCAGCATCAATGCCCA |
| | | TGGGAGCGTCCTGATCAAGGCCACCCACGTGGGCAATGATACAAC |
| | | TCTCGCCCAGATCGTCAAGCTAGTGGAAGAGGCTCAGATGTCTAA |
| | | AGCTCCAATCCAGCAACTGGCTGACCGCTTCAGCGGCTACTTTGTC |
| | | CCCTTTATCATCATCATGAGCACCCTCACCCTGGTGGTGTGGATAG |
| | | TCATAGGCTTCATTGACTTTGGCGTGGTGCAGAGATACTTCCCCAA |
| | | CCCTAACAAACATATCTCCCAAACCGAGGTCATTATCAGATTCGCC |
| | | TTTCAGACATCTATCACTGTGCTGTGCATAGCCTGCCCTTGCTCTCT |
| | | GGGCCTGGCCACCCCCACCGCCGTGATGGTTGGAACCGGGGTCGC |
| | | TGCACAAAATGGGATCCTGATAAAAGGGGGCAAACCCCTGGAAAT |
| | | GGCACATAAGATTAAAACAGTTATGTTTGATAAGACAGGCACCAT |
| | | TACTCACGGTGTCCCTCGAGTCATGAGAGTGCTGCTGCTGGGGGAC |
| | | GTGGCTACACTGCCACTGCGCAAGGTGCTGGCTGTGGTGGGCACA |
| | | GCGGAGGCCTCCTCAGAACACCCATTAGGCGTAGCTGTCACAAAA |
| | | TACTGTAAGGAGGAGCTGGGGACAGAGACCCTCGGCTATTGTACA |
| | | GACTTCCAGGCCGTGCCTGGCTGCGGCATCGGCTGTAAGGTCAGC |
| | | AATGTGGAGGGCATCCTGGCACACAGCGAGAGACCCCTCTCTGCC |
| | | CCAGCCTCCCACCTGAATGAAGCTGGGTCTCTGCCTGCTGAGAAGG |
| | | ACGCCGTCCCTCAGACCTTCTCCGTGTTGATTGGGAACAGAGAGTG |
| | | GCTTAGAAGGAATGGCCTGACCATCAGCTCTGACGTCTCGGATGCC |
| | | ATGACAGATCACGAGATGAAAGGCCAGACAGCCATACTGGTTGCC |
| | | ATCGACGGCGTGCTCTGTGGCATGATTGCCATCGCTGATGCTGTTA |
| | | AACAGGAAGCTGCGCTGGCAGTCCACACCCTCCAGTCCATGGGAG |
| | | TGGACGTGGTCCTGATTACTGGAGACAACAGGAAAACCGCTCGGG |
| | | CCATCGCTACCCAAGTGGGGATAAATAAGGTTTTTGCAGAAGTCTT |
| | | ACCCTCCCACAAAGTAGCCAAAGTGCAGGAGCTCCAGAACAAGGG |
| | | TAAAAAGGTGGCAATGGTGGGAGATGGTGTGAATGACAGCCCAGC |
| | | TCTCGCGCAGGCTGACATGGGCGTAGCCATAGGTACTGGAACAGA |
| | | TGTCGCGATTGAAGCCGCTGACGTAGTCCTCATCCGAAACGATTTG |
| | | CTGGATGTGGTGGCCAGCATCCATCTGTCCAAGCGTACCGTGCGCC |
| | | GGATCCGCATCAACCTGGTCTTGGCCCTCATCTATAATCTCGTGGG |
| | | CATCCCCATCGCTGCTGGGGTCTTCATGCCCATCGGCATTGTGCTG |
| | | CAGCCCTGGATGGGAAGCGCTGCCATGGCTGCTTCTTCTGTCTCCG |
| | | TTGTGCTGTCCAGCCTACAGCTGAAGTGCTATAAAAAGCCAGACCT |
| | | CGAGCGCTACGAAGCCCAGGCGCATGGCCATATGAAGCCCCTCAC |
| | | GGCTTCCCAGGTCTCGGTTCACATTGGGATGGATGACAGATGGCG |
| | | GGACTCCCCACGCGCCACACCCTGGGACCAGGTATCCTATGTGAG |
| | | CCAGGTGAGCCTCAGCTCCCTGACATCAGATAAACCGTCTAGGCA |
| | | CTCAGCTGCCGCAGATGATGACGGTGACAAATGGAGCCTCCTCCT |
| | | GAACGGCCGCGACGAGGAGCAGTACATCTAAGAATTCGATATCAA |
| | | GCTTATCGATAATCAACCTCTGGATTACAAAATTTGTGAAAGATTG |
| | | ACTGGTATTCTTAACTATGTTGCTCCTTTTACGCTATGTGGATACGC |
| | | TGCTTTAATGCCTTTGTATCATGCTATTGCTTCCCGTATGGCTTTCA |
| | | TTTTCTCCTCCTTGTATAAATCCTGGTTAGTTCTTGCCACGGCGGAA |
| | | CTCATCGCCGCCTGCCTTGCCCGCTGCTGGACAGGGGCTCGGCTGT |
| | | TGGGCACTGACAATTCCGTGGTGTTCACCTGCAGGAATAAAAGAT |
| | | CTTTATTTTCATTAGATCTGTGTGTTGGTTTTTTGTGTGTCTAGAGT |
| | | AGATAAGTAGCATGGCGGGTTAATCATTAACTACAAGGAACCCCT |
| | | AGTGATGGAGTTGGCCACTCCCTCTCTGCGCGCTCGCTCGCTCACT |
| | | GAGGCCGGGCGACCAAAGGTCGCCCGACGCCCGGGCTTTGCCCGG |
| | | GCGGCCTCAGTGAGCGAGCGAGCGCGCAGAGAGGGAGTGGCCAA |
| 88 | A35 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCAAGATGGCGGCGCCT |
| | | GAGCAGGAGAGACAGATCACAGCCAGAGAAGGGGCCAGTCGGAA |
| | | AATCTTATCTAAGCTTTCTTTGCCTACCCGTGCCTGGGAACCAGCA |
| | | ATGAAGAAGAGTTTTGCTTTTGACAATGTTGGCTATGAAGGTGGTC |
| | | TGGATGGCCTGGGCCCTTCTTCTCAGGTGGCCCCTGATGGAGCCGA |
| | | AGGGAGTGGGACAGATCACAGGTCTTCCAGTTCTCATTCCCCTGGC |
| | | TCCCCACCGAGAAACCAGGTCCAGGGCACATGCAGTAAGTGCTTC |
| | | TTACAGATCAAAGGCATGACCTGTGCATCCTGTGTGTCTAACATAG |
| | | AAAGGAATCTGCAGAAAGAAGCTGGTGTTCTCTCCGTGTTGGTTGC |
| | | CTTGATGGCAGGAAAGGCAGAGATCAAGTATGACCCAGAGGTCAT |
| | | CCAGCCCCTCGAGATAGCTCAGTTCATCCAGGACCTGGGTTTTGAG |
| | | GCAGCAGTCATGGAGGACTACGCAGGCTCCGATGGCAACATTGAG |
| | | CTGACAATCACAGGGATGACCTGCGCGTCCTGTGTCCACAACATA |
| | | GAGTCCAAACTCACGAGGACAAATGGCATCACTTATGCCTCCGTTG |
| | | CCCTTGCCACCAGCAAAGCCCTTGTTAAGTTTGACCCGGAAATTAT |
| | | CGGTCCACGGGATATTATCAAAATTATTGAGGAAATTGGCTTTCAT |
| | | GCTTCCCTGGCCCAGAGAAACCCCAACGCTCATCACTTGGACCACA |
| | | AGATGGAAATAAAGCAGTGGAAGAAGTCTTTTCTTGTGAGTCTGTT |
| | | TTTCTGTATTCCTGTAATGGGGCTGATGATATATATGATGGTTATG |
| | | GACCACCACTTTGCAACTCTTCACCATAATCAAAACATGAGTAAAG |
| | | AAGAAATGATCAACCTTCATTCTTCTATGTTCCTGGAGCGCCAGAT |
| | | TCTTCCAGGATTGTCTGTTATGAATTTGCTGTCCTTTTTATTGTGTG |
| | | TACCTGTACAGTTTTTCGGAGGCTGGTACTTCTACATTCAGGCTTAT |
| | | AAAGCACTGAAGCATAAGACAGCAAATATGGACGTACTGATTGTG |
| | | CTGGCAACCACCATTGCATTTGCCTACTCTTTGATTATTCTTCTAGT |
| | | TGCAATGTATGAGAGAGCCAAAGTGAACCCTATTACTTTCTTTGAC |
| | | ACACCCCCTATGCTGTTTGTGTTTATTGCACTAGGCCGATGGCTGG |
| | | AACATATAGCAAAGGGCAAAACATCAGAGGCTCTTGCAAAGTTAA |
| | | TTTCACTACAAGCTACAGAAGCAACTATTGTAACTCTTGATTCTGA |
| | | TAATATCCTCCTCAGTGAAGAACAAGTGGATGTGGAACTTGTACA |
| | | ACGTGGAGATATCATTAAAGTAGTTCCAGGAGGCAAATTTCCAGT |
| | | GGATGGTCGTGTTATTGAAGGACATTCTATGGTAGATGAGTCCCTC |
| | | ATCACAGGGGAGGCAATGCCTGTGGCTAAGAAACCTGGCAGCACA |
| | | GTGATTGCTGGTTCCATTAACCAGAACGGGTCACTGCTTATCTGCG |
| | | CAACACATGTTGGAGCAGACACAACCCTTTCTCAAATTGTCAAACT |
| | | TGTGGAAGAGGCACAAACATCAAAGGCTCCTATCCAGCAGTTTGC |
| | | AGACAAACTCAGTGGCTATTTTGTTCCTTTTATTGTTTTTGTTTCCA |
| | | TTGCCACCCTCTTGGTATGGATTGTAATTGGATTTCTGAATTTTGAA |
| | | ATTGTGGAAACCTACTTTCCTGGCTACAATAGAAGTATCTCCCGAA |
| | | CAGAAACGATAATACGATTTGCTTTCCAAGCCTCTATCACAGTTCT |
| | | GTGTATTGCATGTCCCTGTTCACTGGGACTGGCCACTCCAACTGCT |
| | | GTGATGGTGGGTACAGGAGTAGGTGCTCAAAATGGCATACTAATA |
| | | AAAGGTGGAGAGCCATTGGAGATGGCTCATAAGGTAAAGGTAGTG |
| | | GTATTTGATAAGACTGGAACCATTACTCACGGAACCCCAGTGGTG |
| | | AATCAAGTAAAGGTTCTAACTGAAAGTAACAGAATATCACACCAT |
| | | AAAATCTTGGCCATTGTGGGAACTGCTGAAAGTAACAGTGAACAC |
| | | CCTCTAGGAACAGCCATAACCAAATATTGCAAACAGGAGCTGGAC |
| | | ACTGAAACCTTGGGTACCTGCATAGATTTCCAGGTTGTGCCAGGCT |
| | | GTGGTATTAGCTGTAAAGTCACCAATATTGAAGGCTTGCTACATAA |
| | | GAATAACTGGAATATAGAGGACAATAATATTAAAAATGCATCCCT |
| | | GGTTCAAATTGATGCCAGTAATGAACAGTCATCAACTTCGTCTTCC |
| | | ATGATTATTGATGCCCAGATCTCAAATGCTCTTAATGCTCAGCAGT |
| | | ATAAAGTCCTCATTGGTAACCGGGAGTGGATGATTAGAAATGGTC |
| | | TTGTCATTAATAACGATGTAAATGATTTCATGACTGAACATGAGAG |
| | | AAAAGGTCGGACTGCTGTATTAGTAGCAGTTGATGATGAGCTGTGT |
| | | GGCTTGATAGCCATTGCAGACACAGTGAAGCCTGAAGCAGAACTG |
| | | GCTATCCATATTCTGAAATCTATGGGCTTAGAAGTAGTTCTGATGA |
| | | CTGGAGACAACAGTAAAACAGCTAGATCTATTGCTTCTCAGGTTGG |
| | | CATTACTAAGGTGTTTGCTGAAGTTCTACCTTCTCACAAGGTTGCT |
| | | AAAGTGAAGCAACTTCAAGAGGAGGGGAAACGGGTAGCAATGGT |
| | | GGGAGATGGAATCAATGACTCCCCAGCTCTGGCAATGGCTAATGT |
| | | GGGAATTGCTATTGGCACAGGCACAGATGTAGCCATTGAAGCAGC |
| | | TGATGTGGTTTTGATAAGGAATGATCTTCTGGATGTAGTGGCAAGT |
| | | ATTGACTTATCAAGAAAGACAGTCAAGAGGATTCGGATAAATTTT |
| | | GTCTTTGCTCTAATTTATAATCTGGTTGGAATTCCCATAGCTGCTGG |
| | | AGTTTTTATGCCCATTGGTTTGGTTTTGCAGCCCTGGATGGGATCTG |
| | | CAGCAATGGCTGCTTCATCTGTTTCTGTAGTACTTTCTTCTCTCCAG |
| | | CTCAAGTGCTATAAGAAGCCTGACCTGGAGAGGTATGAGGCACAG |
| | | GCGCATGGCCACATGAAGCCCCTGACGGCATCCCAGGTCAGTGTG |
| | | CACATAGGCATGGATGACAGGTGGCGGGACTCCCCCAGGGCCACA |
| | | CCATGGGACCAGGTCAGCTATGTCAGCCAGGTGTCGCTGTCCTCCC |
| | | TGACGTCCGACAAGCCATCTCGGCACAGCGCTGCAGCAGACGATG |
| | | ATGGGGACAAGTGGTCTCTGCTCCTGAATGGCAGGGATGAGGAGC |
| | | AGTACATCTGAGAATTCGATATCAAGCTTATCGATAATCAACCTCT |
| | | GGATTACAAAATTTGTGAAAGATTGACTGGTATTCTTAACTATGTT |
| | | GCTCCTTTTACGCTATGTGGATACGCTGCTTTAATGCCTTTGTATCA |
| | | TGCTATTGCTTCCCGTATGGCTTTCATTTTCTCCTCCTTGTATAAAT |
| | | CCTGGTTAGTTCTTGCCACGGCGGAACTCATCGCCGCCTGCCTTGC |
| | | CCGCTGCTGGACAGGGGCTCGGCTGTTGGGCACTGACAATTCCGTG |
| | | GTGTTCACCTGCAGGAATAAAAGATCTTTATTTTCATTAGATCTGT |
| | | GTGTTGGTTTTTTGTGTGTCTAGAGTAGATAAGTAGCATGGCGGGT |
| | | TAATCATTAACTACAAGGAACCCCTAGTGATGGAGTTGGCCACTCC |
| | | CTCTCTGCGCGCTCGCTCGCTCACTGAGGCCGGGCGACCAAAGGTC |
| | | GCCCGACGCCCGGGCTTTGCCCGGGCGGCCTCAGTGAGCGAGCGA |
| | | GCGCGCAGAGAGGGAGTGGCCAA |
| 89 | A36 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCAAGATGGCGGCGCCT |
| | | GAGCAGGAGAGACAGATCACAGCCAGAGAAGGGGCCAGTCGGAA |
| | | AATCTTATCTAAGCTTTCTTTGCCTACCCGTGCCTGGGAACCAGCA |
| | | ATGAAGAAGAGTTTTGCTTTTGACAATGTTGGCTATGAAGGTGGTC |
| | | TGGATGGCCTGGGCCCTAGCAGCCAGGTGGCTACCACCCTGATTGC |
| | | CATTGCCGGCATGACCTGCGCCAGCTGCGTGCATAGCATTGAGGG |
| | | CATGATTAGCCAGCTGGAGGGCGTGCAGCAGATTTCCGTGAGCCT |
| | | GGCTGAGGGCACCGCTACCGTGCTGTATAATCCCTCCGTGATTAGC |
| | | CCCGAAGAACTGCGCGCTGCCATTGAAGATATGGGCTTTGAAGCC |
| | | TCCGTGGTGTCCGAAAGCTGCAGCACCAATCCCCTGGGCAATCATT |
| | | CCGCCGGCAATAGCATGGTGCAGACCACCGACGGCACCCCCACCT |
| | | CCGTGCAGGAAGTGGCTCCCCATACCGGCCGCCTGCCCGCCAATC |
| | | ATGCCCCTGATATTCTGGCTAAAAGCCCCCAGAGCACCCGCGCCGT |
| | | GGCTCCTCAGAAATGCTTTCTGCAGATTAAGGGCATGACCTGCGCT |
| | | AGCTGCGTGAGCAATATTGAACGCAATCTGCAGAAAGAAGCCGGC |
| | | GTGCTGTCCGTGCTGGTGGCCCTGATGGCCGGCAAAGCTGAAATC |
| | | AAATATGATCCCGAAGTGATTCAGCCCCTGGAAATTGCCCAGTTCA |
| | | TTCAGGATCTGGGCTTTGAGGCTGCCGTGATGGAAGATTATGCCGG |
| | | CTCCGACGGCAATATTGAACTGACCATTACCGGCATGACCTGCGCC |
| | | AGCTGCGTGCATAATATTGAAAGCAAACTGACCCGCACCAACGGC |
| | | ATTACCTATGCTTCCGTGGCCCTGGCTACCAGCAAAGCCCTGGTGA |
| | | AATTTGATCCTGAAATTATCGGCCCCCGCGATATTATCAAAATTAT |
| | | TGAAGAAATCGGCTTTCACGCCAGCCTGGCTCAGCGCAATCCCAAT |
| | | GCCCATCATCTGGATCATAAGATGGAAATCAAACAGTGGAAGAAA |
| | | AGCTTTCTGTGCAGCCTGGTGTTCGGCATTCCTGTGATGGCCCTGA |
| | | TGATCTATATGCTGATTCCCAGCAATGAACCCCATCAGAGCATGGT |
| | | GCTGGATCATAATATTATTCCCGGCCTGAGCATTCTGAATCTGATT |
| | | TTCTTTATCCTGTGCACCTTTGTGCAGCTGCTGGGCGGCTGGTATTT |
| | | CTATGTGCAGGCTTATAAAAGCCTGCGCCACCGCTCCGCCAATATG |
| | | GATGTGCTGATTGTGCTGGCCACCAGCATTGCTTATGTCTATAGCC |
| | | TGGTCATTCTGGTGGTGGCCGTGGCCGAGAAAGCTGAACGCAGCC |
| | | CCGTGACCTTCTTTGATACCCCTCCCATGCTGTTCGTGTTTATCGCT |
| | | CTGGGCCGCTGGCTGGAACATCTGGCCAAAAGCAAAACCTCCGAA |
| | | GCCCTGGCTAAACTGATGAGCCTGCAGGCCACCGAAGCCACCGTG |
| | | GTGACCCTGGGCGAAGATAATCTGATTATCCGCGAAGAACAGGTG |
| | | CCTATGGAACTGGTGCAGCGCGGCGATATTGTGAAAGTGGTGCCC |
| | | GGCGGCAAATTTCCCGTGGACGGCAAAGTGCTGGAGGGCAATACC |
| | | ATGGCTGATGAATCCCTGATTACCGGCGAAGCTATGCCTGTGACCA |
| | | AGAAACCCGGCAGCACCGTGATTGCCGGCAGCATTAATGCTCACG |
| | | GCTCCGTGCTGATCAAGGCTACCCATGTCGGCAATGATACCACCCT |
| | | GGCCCAGATTGTGAAACTGGTGGAAGAAGCCCAGATGAGCAAAGC |
| | | CCCTATTCAGCAGCTGGCTGACCGCTTTTCCGGCTATTTTGTGCCCT |
| | | TTATCATTATTATGAGCACCCTGACCCTGGTGGTGTGGATTGTGAT |
| | | CGGCTTCATTGATTTCGGCGTGGTGCAGAGATATTTTCCTAATCCC |
| | | AACAAACATATTAGCCAGACCGAAGTGATTATCCGCTTTGCTTTTC |
| | | AGACCAGCATTACCGTGCTGTGCATTGCTTGCCCCTGCAGCCTGGG |
| | | CCTGGCTACCCCCACCGCCGTCATGGTCGGCACCGGCGTGGCTGCC |
| | | CAGAACGGCATTCTGATTAAGGGCGGCAAACCTCTGGAAATGGCC |
| | | CACAAAATCAAAACCGTGATGTTTGATAAAACCGGCACCATTACC |
| | | CACGGCGTGCCCCGCGTGATGCGCGTGCTGCTGCTGGGCGATGTG |
| | | GCTACCCTGCCCCTGCGCAAAGTGCTGGCCGTGGTCGGCACCGCCG |
| | | AAGCTAGCTCCGAACATCCCCTGGGCGTGGCCGTGACCAAATATT |
| | | GCAAAGAAGAACTGGGCACCGAAACCCTGGGCTATTGCACCGATT |
| | | TTCAGGCCGTCCCTGGCTGCGGCATCGGCTGCAAAGTGAGCAATGT |
| | | GGAGGGCATTCTGGCCCATTCCGAAAGACCCCTGTCCGCCCCTGCT |
| | | AGCCATCTGAATGAAGCCGGCAGCCTGCCTGCTGAGAAAGATGCC |
| | | GTGCCCCAGACCTTTTCCGTGCTGATCGGCAACCGCGAATGGCTGC |
| | | GCCGCAACGGCCTGACCATTAGCTCCGATGTCTCCGATGCCATGAC |
| | | CGATCATGAAATGAAAGGCCAGACCGCCATTCTGGTGGCCATTGA |
| | | CGGCGTGCTGTGCGGCATGATTGCCATTGCCGATGCCGTGAAACA |
| | | GGAGGCTGCCCTGGCTGTGCATACCCTGCAGAGCATGGGCGTGGA |
| | | TGTGGTGCTGATTACCGGCGATAACCGCAAAACCGCTCGCGCCATT |
| | | GCCACCCAGGTCGGCATTAACAAAGTGTTTGCTGAAGTGCTGCCCA |
| | | GCCATAAAGTGGCTAAAGTGCAGGAACTGCAGAACAAAGGCAAG |
| | | AAAGTGGCTATGGTCGGCGACGGCGTGAATGATAGCCCTGCCCTG |
| | | GCCCAGGCCGATATGGGCGTGGCTATCGGCACCGGCACCGATGTG |
| | | GCTATTGAGGCTGCCGATGTGGTGCTGATCCGCAATGATCTGCTGG |
| | | ATGTGGTGGCTAGCATTCATCTGAGCAAACGCACCGTCCGCCGCAT |
| | | CCGCATTAATCTGGTGCTGGCCCTGATCTATAATCTGGTCGGCATT |
| | | CCCATCGCCGCCGGCGTGTTTATGCCCATCGGCATTGTGCTGCAGC |
| | | CCTGGATGGGCAGCGCCGCTATGGCCGCTAGCTCCGTGTCCGTGGT |
| | | GCTGAGCAGCCTGCAGCTGAAATGCTATAAGAAACCCGATCTGGA |
| | | AAGATATGAAGCTCAGGCTCACGGCCATATGAAACCCCTGACCGC |
| | | CAGCCAGGTGTCCGTGCATATCGGCATGGATGACCGCTGGCGCGA |
| | | TAGCCCCCGCGCCACCCCTTGGGATCAGGTGAGCTATGTGAGCCA |
| | | GGTGTCCCTGAGCTCCCTGACCTCCGATAAACCCTCCCGCCATTCC |
| | | GCCGCTGCCGATGATGACGGCGATAAGTGGAGCCTGCTGCTGAAC |
| | | GGCCGCGATGAAGAACAGTATATCTAAGTCCATAAAGTAGGAAAC |
| | | ACTACATCATCCATAAAGTAGGAAACACTACATCATCCATAAAGT |
| | | AGGAAACACTACATCATCCATAAAGTAGGAAACACTACAGAATTC |
| | | GATATCAAGCTTATCGATAATCAACCTCTGGATTACAAAATTTGTG |
| | | AAAGATTGACTGGTATTCTTAACTATGTTGCTCCTTTTACGCTATGT |
| | | GGATACGCTGCTTTAATGCCTTTGTATCATGCTATTGCTTCCCGTAT |
| | | GGCTTTCATTTTCTCCTCCTTGTATAAATCCTGGTTAGTTCTTGCCA |
| | | CGGCGGAACTCATCGCCGCCTGCCTTGCCCGCTGCTGGACAGGGG |
| | | CTCGGCTGTTGGGCACTGACAATTCCGTGGTGTTCACCTGCAGGAA |
| | | TAAAAGATCTTTATTTTCATTAGATCTGTGTGTTGGTTTTTTGTGTG |
| | | TCTAGAGTAGATAAGTAGCATGGCGGGTTAATCATTAACTACAAG |
| | | GAACCCCTAGTGATGGAGTTGGCCACTCCCTCTCTGCGCGCTCGCT |
| | | CGCTCACTGAGGCCGGGCGACCAAAGGTCGCCCGACGCCCGGGCT |
| | | TTGCCCGGGCGGCCTCAGTGAGCGAGCGAGCGCGCAGAGAGGGAG |
| | | TGGCCAA |
| 90 | A37 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTGCTGGTGCTTCCCCAGGCTGGAGA |
| | | TTGAGTTAATATTAACAGGCCCAAGGCGATGTGGGCTTGTGCAATC |
| | | ATAGGCCCGGCCTCGCCAGGTCACCTGAGGAGTTAATGAATACAT |
| | | ATCTCCTGCCGCGTTCGCCCCGCCCCCGACGGTATCGATAAGCTTA |
| | | GGAGCTTGGGCTGCAGGTCGAGGGCACTGGGAGGATGTTGAGTAA |
| | | GATGGAAAACTACTGATGACCCTTGCAGAGACAGAGTATTAGGAC |
| | | ATGTTTGAACAGGGGCCGGGCGATCAGCAGGTAGCTCTAGAGGAT |
| | | CCCCGTCTGTCTGCACATTTCGTAGAGCGAGTGTTCCGATACTCTA |
| | | ATCTCCCTAGGCAAGGTTCATATTTGTGTAGGTTACTTATTCTCCTT |
| | | TTGTTGACTAAGTCAATAATCAGAATCAGCAGGTTTGGAGTCAGCT |
| | | TGGCAGGGATCAGCAGCCTGGGTTGGAAGGAGGGGGTATAAAAGC |
| | | CCCTTCACCAGGAGAAGCCGTCACACAGATCCACAAGCTCGAGCC |
| | | GAGTGGTCGTGCCTCCATAGAAGCGGCCGCAAGATGGCGGCGCCT |
| | | GAGCAGGAGAGACAGATCACAGCCAGAGAAGGGGCCAGTCGGAA |
| | | AATCTTATCTAAGCTTTCTTTGCCTACCCGTGCCTGGGAACCAGCA |
| | | ATGAAGAAGAGTTTTGCTTTTGACAATGTTGGCTATGAAGGTGGTC |
| | | TGGATGGCCTGGGCCCTTCTTCTCAGGTGGCCCCTGATGGAGCCGA |
| | | AGGGAGTGGGACAGATCACAGGTCTTCCAGTTCTCATTCCCCTGGC |
| | | TCCCCACCGAGAAACCAGGTCCAGGGCACATGCAGTAAGTGCTTC |
| | | TTACAGATCAAAGGCATGACCTGTGCATCCTGTGTGTCTAACATAG |
| | | AAAGGAATCTGCAGAAAGAAGCTGGTGTTCTCTCCGTGTTGGTTGC |
| | | CTTGATGGCAGGAAAGGCAGAGATCAAGTATGACCCAGAGGTCAT |
| | | CCAGCCCCTCGAGATAGCTCAGTTCATCCAGGACCTGGGTTTTGAG |
| | | GCAGCAGTCATGGAGGACTACGCAGGCTCCGATGGCAACATTGAG |
| | | CTGACAATCACAGGGATGACCTGCGCGTCCTGTGTCCACAACATA |
| | | GAGTCCAAACTCACGAGGACAAATGGCATCACTTATGCCTCCGTTG |
| | | CCCTTGCCACCAGCAAAGCCCTTGTTAAGTTTGACCCGGAAATTAT |
| | | CGGTCCACGGGATATTATCAAAATTATTGAGGAAATTGGCTTTCAT |
| | | GCTTCCCTGGCCCAGAGAAACCCCAACGCTCATCACTTGGACCACA |
| | | AGATGGAAATAAAGCAGTGGAAGAAGTCTTTTCTTGTGAGTCTGTT |
| | | TTTCTGTATTCCTGTAATGGGGCTGATGATATATATGATGGTTATG |
| | | GACCACCACTTTGCAACTCTTCACCATAATCAAAACATGAGTAAAG |
| | | AAGAAATGATCAACCTTCATTCTTCTATGTTCCTGGAGCGCCAGAT |
| | | TCTTCCAGGATTGTCTGTTATGAATTTGCTGTCCTTTTTATTGTGTG |
| | | TACCTGTACAGTTTTTCGGAGGCTGGTACTTCTACATTCAGGCTTAT |
| | | AAAGCACTGAAGCATAAGACAGCAAATATGGACGTACTGATTGTG |
| | | CTGGCAACCACCATTGCATTTGCCTACTCTTTGATTATTCTTCTAGT |
| | | TGCAATGTATGAGAGAGCCAAAGTGAACCCTATTACTTTCTTTGAC |
| | | ACACCCCCTATGCTGTTTGTGTTTATTGCACTAGGCCGATGGCTGG |
| | | AACATATAGCAAAGGGCAAAACATCAGAGGCTCTTGCAAAGTTAA |
| | | TTTCACTACAAGCTACAGAAGCAACTATTGTAACTCTTGATTCTGA |
| | | TAATATCCTCCTCAGTGAAGAACAAGTGGATGTGGAACTTGTACA |
| | | ACGTGGAGATATCATTAAAGTAGTTCCAGGAGGCAAATTTCCAGT |
| | | GGATGGTCGTGTTATTGAAGGACATTCTATGGTAGATGAGTCCCTC |
| | | ATCACAGGGGAGGCAATGCCTGTGGCTAAGAAACCTGGCAGCACA |
| | | GTGATTGCTGGTTCCATTAACCAGAACGGGTCACTGCTTATCTGCG |
| | | CAACACATGTTGGAGCAGACACAACCCTTTCTCAAATTGTCAAACT |
| | | TGTGGAAGAGGCACAAACATCAAAGGCTCCTATCCAGCAGTTTGC |
| | | AGACAAACTCAGTGGCTATTTTGTTCCTTTTATTGTTTTTGTTTCCA |
| | | TTGCCACCCTCTTGGTATGGATTGTAATTGGATTTCTGAATTTTGAA |
| | | ATTGTGGAAACCTACTTTCCTGGCTACAATAGAAGTATCTCCCGAA |
| | | CAGAAACGATAATACGATTTGCTTTCCAAGCCTCTATCACAGTTCT |
| | | GTGTATTGCATGTCCCTGTTCACTGGGACTGGCCACTCCAACTGCT |
| | | GTGATGGTGGGTACAGGAGTAGGTGCTCAAAATGGCATACTAATA |
| | | AAAGGTGGAGAGCCATTGGAGATGGCTCATAAGGTAAAGGTAGTG |
| | | GTATTTGATAAGACTGGAACCATTACTCACGGAACCCCAGTGGTG |
| | | AATCAAGTAAAGGTTCTAACTGAAAGTAACAGAATATCACACCAT |
| | | AAAATCTTGGCCATTGTGGGAACTGCTGAAAGTAACAGTGAACAC |
| | | CCTCTAGGAACAGCCATAACCAAATATTGCAAACAGGAGCTGGAC |
| | | ACTGAAACCTTGGGTACCTGCATAGATTTCCAGGTTGTGCCAGGCT |
| | | GTGGTATTAGCTGTAAAGTCACCAATATTGAAGGCTTGCTACATAA |
| | | GAATAACTGGAATATAGAGGACAATAATATTAAAAATGCATCCCT |
| | | GGTTCAAATTGATGCCAGTAATGAACAGTCATCAACTTCGTCTTCC |
| | | ATGATTATTGATGCCCAGATCTCAAATGCTCTTAATGCTCAGCAGT |
| | | ATAAAGTCCTCATTGGTAACCGGGAGTGGATGATTAGAAATGGTC |
| | | TTGTCATTAATAACGATGTAAATGATTTCATGACTGAACATGAGAG |
| | | AAAAGGTCGGACTGCTGTATTAGTAGCAGTTGATGATGAGCTGTGT |
| | | GGCTTGATAGCCATTGCAGACACAGTGAAGCCTGAAGCAGAACTG |
| | | GCTATCCATATTCTGAAATCTATGGGCTTAGAAGTAGTTCTGATGA |
| | | CTGGAGACAACAGTAAAACAGCTAGATCTATTGCTTCTCAGGTTGG |
| | | CATTACTAAGGTGTTTGCTGAAGTTCTACCTTCTCACAAGGTTGCT |
| | | AAAGTGAAGCAACTTCAAGAGGAGGGGAAACGGGTAGCAATGGT |
| | | GGGAGATGGAATCAATGACTCCCCAGCTCTGGCAATGGCTAATGT |
| | | GGGAATTGCTATTGGCACAGGCACAGATGTAGCCATTGAAGCAGC |
| | | TGATGTGGTTTTGATAAGGAATGATCTTCTGGATGTAGTGGCAAGT |
| | | ATTGACTTATCAAGAAAGACAGTCAAGAGGATTCGGATAAATTTT |
| | | GTCTTTGCTCTAATTTATAATCTGGTTGGAATTCCCATAGCTGCTGG |
| | | AGTTTTTATGCCCATTGGTTTGGTTTTGCAGCCCTGGATGGGATCTG |
| | | CAGCAATGGCTGCTTCATCTGTTTCTGTAGTACTTTCTTCTCTCCAG |
| | | CTCAAGTGCTATAAGAAGCCTGACCTGGAGAGGTATGAGGCACAG |
| | | GCGCATGGCCACATGAAGCCCCTGACGGCATCCCAGGTCAGTGTG |
| | | CACATAGGCATGGATGACAGGTGGCGGGACTCCCCCAGGGCCACA |
| | | CCATGGGACCAGGTCAGCTATGTCAGCCAGGTGTCGCTGTCCTCCC |
| | | TGACGTCCGACAAGCCATCTCGGCACAGCGCTGCAGCAGACGATG |
| | | ATGGGGACAAGTGGTCTCTGCTCCTGAATGGCAGGGATGAGGAGC |
| | | AGTACATCTGAGAATTCGATATCAAGCTTATCGATAATCAACCTCT |
| | | GGATTACAAAATTTGTGAAAGATTGACTGGTATTCTTAACTATGTT |
| | | GCTCCTTTTACGCTATGTGGATACGCTGCTTTAATGCCTTTGTATCA |
| | | TGCTATTGCTTCCCGTATGGCTTTCATTTTCTCCTCCTTGTATAAAT |
| | | CCTGGTTGCTGTCTCTTTATGAGGAGTTGTGGCCCGTTGTCAGGCA |
| | | ACGTGGCGTGGTGTGCACTGTGTTTGCTGACGCAACCCCCACTGGT |
| | | TGGGGCACCTGCAGGAATAAAAGATCTTTATTTTCATTAGATCTGT |
| | | GTGTTGGTTTTTTGTGTGTCTAGAGTAGATAAGTAGCATGGCGGGT |
| | | TAATCATTAACTACAAGGAACCCCTAGTGATGGAGTTGGCCACTCC |
| | | CTCTCTGCGCGCTCGCTCGCTCACTGAGGCCGGGCGACCAAAGGTC |
| | | GCCCGACGCCCGGGCTTTGCCCGGGCGGCCTCAGTGAGCGAGCGA |
| | | GCGCGCAGAGAGGGAGTGGCCAA |
| 91 | A38 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAATTTTAATAAATACCTCTCTGTCCTTCT |
| | | CCTAGCTTCACCCTTTGTTTGTTGAAAGGAGATACTTTAGTGCAGT |
| | | ATTGCAACAGCTTATGTAGACATGCAACCTTGAGGTCACAGACCTA |
| | | TGAGGGATTGAGACAAAGGCAAATTTAGCAAATATTTTCCAGAGT |
| | | ACTCTGCTGCAATAACCGCGTTCGCCCCGCCCCGGACGGTATCGAT |
| | | AAGCTTAGGAGCTTGGGCTGCAGGTCGAGGGCACTGGGAGGATGT |
| | | TGAGTAAGATGGAAAACTACTGATGACCCTTGCAGAGACAGAGTA |
| | | TTAGGACATGTTTGAACAGGGGCCGGGCGATCAGCAGGTAGCTCT |
| | | AGAGGATCCCCGTCTGTCTGCACATTTCGTAGAGCGAGTGTTCCGA |
| | | TACTCTAATCTCCCTAGGCAAGGTTCATATTTGTGTAGGTTACTTAT |
| | | TCTCCTTTTGTTGACTAAGTCAATAATCAGAATCAGCAGGTTTGGA |
| | | GTCAGCTTGGCAGGGATCAGCAGCCTGGGTTGGAAGGAGGGGGTA |
| | | TAAAAGCCCCTTCACCAGGAGAAGCCGTCACACAGATCCACAAGC |
| | | TCGAGCCGAGTGGTCGTGCCTCCATAGAAGCGGCCGCCACCATGC |
| | | CGGAACAAGAACGCCAGATCACAGCAAGAGAAGGAGCAAGTAGG |
| | | AAGATCCTGAGCAAGCTCTCCCTGCCCACAAGGGCCTGGGAGCCG |
| | | GCCATGAAGAAGTCTTTTGCCTTCGATAATGTCGGTTATGAAGGCG |
| | | GGCTCGATGGCCTGGGCCCCAGCAGCCAGCCCCAGAAGTGTTTCCT |
| | | GCAGATCAAGGGCATGACCTGTGCCAGCTGTGTTAGCAATATCGA |
| | | AAGAAACCTCCAAAAAGAGGCTGGAGTTCTGAGTGTTCTAGTGGC |
| | | TTTAATGGCCGGGAAAGCCGAGATCAAGTATGATCCCGAGGTGAT |
| | | TCAGCCATTGGAGATTGCCCAGTTCATCCAGGATCTGGGCTTCGAG |
| | | GCCGCCGTGATGGAGGATTACGCCGGCTCTGATGGCAACATCGAA |
| | | CTGACCATCACCGGCATGACCTGTGCTTCCTGCGTGCATAATATCG |
| | | AGAGCAAACTGACGCGGACCAATGGAATCACCTATGCCAGCGTCG |
| | | CCCTGGCAACCTCTAAGGCTCTGGTGAAGTTTGACCCCGAAATCAT |
| | | TGGACCTAGAGACATCATCAAAATTATTGAAGAGATTGGTTTCCAC |
| | | GCCAGCTTAGCTCAGCGGAATCCCAACGCACACCATCTTGACCATA |
| | | AGATGGAAATCAAACAGTGGAAGAAGAGCTTCTTGTGTAGCCTGG |
| | | TCTTCGGGATACCCGTCATGGCACTGATGATTTACATGCTCATTCC |
| | | GTCCAACGAGCCCCACCAGTCCATGGTGCTGGACCACAACATCAT |
| | | CCCTGGGCTGTCTATCCTCAACCTGATCTTCTTTATTCTGTGCACAT |
| | | TTGTCCAGCTCTTGGGAGGCTGGTATTTCTATGTACAGGCCTACAA |
| | | ATCCCTGAGGCATCGAAGCGCCAACATGGACGTTTTAATTGTGCTG |
| | | GCTACATCCATTGCCTACGTGTACTCCCTGGTGATCTTAGTGGTTG |
| | | CTGTGGCTGAAAAGGCTGAGAGAAGCCCTGTCACCTTCTTCGACAC |
| | | CCCCCCCATGCTCTTCGTGTTTATTGCACTGGGTCGCTGGCTGGAG |
| | | CATTTAGCCAAGTCCAAGACATCCGAGGCTCTGGCCAAGCTGATGT |
| | | CTCTACAGGCCACGGAGGCCACCGTGGTGACTCTGGGTGAAGACA |
| | | ATTTGATAATACGCGAGGAACAGGTGCCTATGGAGCTTGTGCAGC |
| | | GCGGAGATATCGTCAAGGTGGTGCCCGGCGGCAAGTTCCCAGTGG |
| | | ATGGAAAAGTGTTAGAGGGCAATACCATGGCCGATGAATCCCTCA |
| | | TCACTGGCGAGGCAATGCCGGTGACCAAGAAACCTGGAAGTACTG |
| | | TCATTGCAGGCAGCATCAATGCCCATGGGAGCGTCCTGATCAAGG |
| | | CCACCCACGTGGGCAATGATACAACTCTCGCCCAGATCGTCAAGCT |
| | | AGTGGAAGAGGCTCAGATGTCTAAAGCTCCAATCCAGCAACTGGC |
| | | TGACCGCTTCAGCGGCTACTTTGTCCCCTTTATCATCATCATGAGC |
| | | ACCCTCACCCTGGTGGTGTGGATAGTCATAGGCTTCATTGACTTTG |
| | | GCGTGGTGCAGAGATACTTCCCCAACCCTAACAAACATATCTCCCA |
| | | AACCGAGGTCATTATCAGATTCGCCTTTCAGACATCTATCACTGTG |
| | | CTGTGCATAGCCTGCCCTTGCTCTCTGGGCCTGGCCACCCCCACCG |
| | | CCGTGATGGTTGGAACCGGGGTCGCTGCACAAAATGGGATCCTGA |
| | | TAAAAGGGGGCAAACCCCTGGAAATGGCACATAAGATTAAAACAG |
| | | TTATGTTTGATAAGACAGGCACCATTACTCACGGTGTCCCTCGAGT |
| | | CATGAGAGTGCTGCTGCTGGGGGACGTGGCTACACTGCCACTGCG |
| | | CAAGGTGCTGGCTGTGGTGGGCACAGCGGAGGCCTCCTCAGAACA |
| | | CCCATTAGGCGTAGCTGTCACAAAATACTGTAAGGAGGAGCTGGG |
| | | GACAGAGACCCTCGGCTATTGTACAGACTTCCAGGCCGTGCCTGGC |
| | | TGCGGCATCGGCTGTAAGGTCAGCAATGTGGAGGGCATCCTGGCA |
| | | CACAGCGAGAGACCCCTCTCTGCCCCAGCCTCCCACCTGAATGAA |
| | | GCTGGGTCTCTGCCTGCTGAGAAGGACGCCGTCCCTCAGACCTTCT |
| | | CCGTGTTGATTGGGAACAGAGAGTGGCTTAGAAGGAATGGCCTGA |
| | | CCATCAGCTCTGACGTCTCGGATGCCATGACAGATCACGAGATGA |
| | | AAGGCCAGACAGCCATACTGGTTGCCATCGACGGCGTGCTCTGTG |
| | | GCATGATTGCCATCGCTGATGCTGTTAAACAGGAAGCTGCGCTGGC |
| | | AGTCCACACCCTCCAGTCCATGGGAGTGGACGTGGTCCTGATTACT |
| | | GGAGACAACAGGAAAACCGCTCGGGCCATCGCTACCCAAGTGGGG |
| | | ATAAATAAGGTTTTTGCAGAAGTCTTACCCTCCCACAAAGTAGCCA |
| | | AAGTGCAGGAGCTCCAGAACAAGGGTAAAAAGGTGGCAATGGTG |
| | | GGAGATGGTGTGAATGACAGCCCAGCTCTCGCGCAGGCTGACATG |
| | | GGCGTAGCCATAGGTACTGGAACAGATGTCGCGATTGAAGCCGCT |
| | | GACGTAGTCCTCATCCGAAACGATTTGCTGGATGTGGTGGCCAGCA |
| | | TCCATCTGTCCAAGCGTACCGTGCGCCGGATCCGCATCAACCTGGT |
| | | CTTGGCCCTCATCTATAATCTCGTGGGCATCCCCATCGCTGCTGGG |
| | | GTCTTCATGCCCATCGGCATTGTGCTGCAGCCCTGGATGGGAAGCG |
| | | CTGCCATGGCTGCTTCTTCTGTCTCCGTTGTGCTGTCCAGCCTACAG |
| | | CTGAAGTGCTATAAAAAGCCAGACCTCGAGCGCTACGAAGCCCAG |
| | | GCGCATGGCCATATGAAGCCCCTCACGGCTTCCCAGGTCTCGGTTC |
| | | ACATTGGGATGGATGACAGATGGCGGGACTCCCCACGCGCCACAC |
| | | CCTGGGACCAGGTATCCTATGTGAGCCAGGTGAGCCTCAGCTCCCT |
| | | GACATCAGATAAACCGTCTAGGCACTCAGCTGCCGCAGATGATGA |
| | | CGGTGACAAATGGAGCCTCCTCCTGAACGGCCGCGACGAGGAGCA |
| | | GTACATCTAAGAATTCGATATCAAGCTTATCGATAATCAACCTCTG |
| | | GATTACAAAATTTGTGAAAGATTGACTGGTATTCTTAACTATGTTG |
| | | CTCCTTTTACGCTATGTGGATACGCTGCTTTAATGCCTTTGTATCAT |
| | | GCTATTGCTTCCCGTATGGCTTTCATTTTCTCCTCCTTGTATAAATC |
| | | CTGGTTGCTGTCTCTTTATGAGGAGTTGTGGCCCGTTGTCAGGCAA |
| | | CGTGGCGTGGTGTGCACTGTGTTTGCTGACGCAACCCCCACTGGTT |
| | | GGGGCACCTGCAGGAATAAAAGATCTTTATTTTCATTAGATCTGTG |
| | | TGTTGGTTTTTTGTGTGTCTAGAGTAGATAAGTAGCATGGCGGGTT |
| | | AATCATTAACTACAAGGAACCCCTAGTGATGGAGTTGGCCACTCCC |
| | | TCTCTGCGCGCTCGCTCGCTCACTGAGGCCGGGCGACCAAAGGTCG |
| | | CCCGACGCCCGGGCTTTGCCCGGGCGGCCTCAGTGAGCGAGCGAG |
| | | CGCGCAGAGAGGGAGTGGCCAA |
| 92 | A39 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGACACCCCCTCCACCTTGGACACAGGACGCT |
| | | GTGGTTTCTGAGCCAGGTACAATGACTCCTTTCGGTAAGTGCAGTG |
| | | GAAGCTGTACACTGCCCAGGCAAAGCGTCCGGGCAGCGTAGGCGG |
| | | GCGACTCAGATCCCAGCCAGTGGACTTAGCCCCTGTTTGCTCCTCC |
| | | GATAACTGGGGTGACCTTGGTTAATATTCACCAGCAGCCTCCCCCG |
| | | TTGCCCCTCTGGATCCACTGCTTAAATACGGACGAGGACAGGGCCC |
| | | TGTCTCCTCAGCTTCAGGCACCACCACTGACCTGGGACAGTGAAGC |
| | | GGCCGCCACCGAGTTAATAATTACCAGCGCGGGCCAAATAAATAA |
| | | TCCGCGAGGGGCAGCTGGACTTTGGACTCCGCGCGCTGGTAATTAT |
| | | TAACCTCGCGAATATTGATTCGAGGCCGCGATTGCCGCAATCGCGA |
| | | GGGGCAGGTGACCTTTGCCCAGCGCGCGTTCGCCCCGCCCCGGAC |
| | | GGTATCGATAAGCTTAGGAGCTTGGGCTGCAGGTCGAGGGCACTG |
| | | GGAGGATGTTGAGTAAGATGGAAAACTACTGATGACCCTTGCAGA |
| | | GACAGAGTATTAGGACATGTTTGAACAGGGGCCGGGCGATCAGCA |
| | | GGTAGCTCTAGAGGATCCCCGTCTGTCTGCACATTTCGTAGAGCGA |
| | | GTGTTCCGATACTCTAATCTCCCTAGGCAAGGTTCATATTTGTGTA |
| | | GGTTACTTATTCTCCTTTTGTTGACTAAGTCAATAATCAGAATCAG |
| | | CAGGTTTGGAGTCAGCTTGGCAGGGATCAGCAGCCTGGGTTGGAA |
| | | GGAGGGGGTATAAAAGCCCCTTCACCAGGAGAAGCCGTCACACAG |
| | | ATCCACAAGCTCGAGCCGAGTGGTCGTGCCTCCATAGAAGCGGCC |
| | | GCCACCATGCCGGAACAAGAACGCCAGATCACAGCAAGAGAAGG |
| | | AGCAAGTAGGAAGATCCTGAGCAAGCTCTCCCTGCCCACAAGGGC |
| | | CTGGGAGCCGGCCATGAAGAAGTCTTTTGCCTTCGATAATGTCGGT |
| | | TATGAAGGCGGGCTCGATGGCCTGGGCCCCAGCAGCCAGCCCCAG |
| | | AAGTGTTTCCTGCAGATCAAGGGCATGACCTGTGCCAGCTGTGTTA |
| | | GCAATATCGAAAGAAACCTCCAAAAAGAGGCTGGAGTTCTGAGTG |
| | | TTCTAGTGGCTTTAATGGCCGGGAAAGCCGAGATCAAGTATGATCC |
| | | CGAGGTGATTCAGCCATTGGAGATTGCCCAGTTCATCCAGGATCTG |
| | | GGCTTCGAGGCCGCCGTGATGGAGGATTACGCCGGCTCTGATGGC |
| | | AACATCGAACTGACCATCACCGGCATGACCTGTGCTTCCTGCGTGC |
| | | ATAATATCGAGAGCAAACTGACGCGGACCAATGGAATCACCTATG |
| | | CCAGCGTCGCCCTGGCAACCTCTAAGGCTCTGGTGAAGTTTGACCC |
| | | CGAAATCATTGGACCTAGAGACATCATCAAAATTATTGAAGAGAT |
| | | TGGTTTCCACGCCAGCTTAGCTCAGCGGAATCCCAACGCACACCAT |
| | | CTTGACCATAAGATGGAAATCAAACAGTGGAAGAAGAGCTTCTTG |
| | | TGTAGCCTGGTCTTCGGGATACCCGTCATGGCACTGATGATTTACA |
| | | TGCTCATTCCGTCCAACGAGCCCCACCAGTCCATGGTGCTGGACCA |
| | | CAACATCATCCCTGGGCTGTCTATCCTCAACCTGATCTTCTTTATTC |
| | | TGTGCACATTTGTCCAGCTCTTGGGAGGCTGGTATTTCTATGTACA |
| | | GGCCTACAAATCCCTGAGGCATCGAAGCGCCAACATGGACGTTTT |
| | | AATTGTGCTGGCTACATCCATTGCCTACGTGTACTCCCTGGTGATC |
| | | TTAGTGGTTGCTGTGGCTGAAAAGGCTGAGAGAAGCCCTGTCACCT |
| | | TCTTCGACACCCCCCCCATGCTCTTCGTGTTTATTGCACTGGGTCGC |
| | | TGGCTGGAGCATTTAGCCAAGTCCAAGACATCCGAGGCTCTGGCC |
| | | AAGCTGATGTCTCTACAGGCCACGGAGGCCACCGTGGTGACTCTG |
| | | GGTGAAGACAATTTGATAATACGCGAGGAACAGGTGCCTATGGAG |
| | | CTTGTGCAGCGCGGAGATATCGTCAAGGTGGTGCCCGGCGGCAAG |
| | | TTCCCAGTGGATGGAAAAGTGTTAGAGGGCAATACCATGGCCGAT |
| | | GAATCCCTCATCACTGGCGAGGCAATGCCGGTGACCAAGAAACCT |
| | | GGAAGTACTGTCATTGCAGGCAGCATCAATGCCCATGGGAGCGTC |
| | | CTGATCAAGGCCACCCACGTGGGCAATGATACAACTCTCGCCCAG |
| | | ATCGTCAAGCTAGTGGAAGAGGCTCAGATGTCTAAAGCTCCAATC |
| | | CAGCAACTGGCTGACCGCTTCAGCGGCTACTTTGTCCCCTTTATCA |
| | | TCATCATGAGCACCCTCACCCTGGTGGTGTGGATAGTCATAGGCTT |
| | | CATTGACTTTGGCGTGGTGCAGAGATACTTCCCCAACCCTAACAAA |
| | | CATATCTCCCAAACCGAGGTCATTATCAGATTCGCCTTTCAGACAT |
| | | CTATCACTGTGCTGTGCATAGCCTGCCCTTGCTCTCTGGGCCTGGC |
| | | CACCCCCACCGCCGTGATGGTTGGAACCGGGGTCGCTGCACAAAA |
| | | TGGGATCCTGATAAAAGGGGGCAAACCCCTGGAAATGGCACATAA |
| | | GATTAAAACAGTTATGTTTGATAAGACAGGCACCATTACTCACGGT |
| | | GTCCCTCGAGTCATGAGAGTGCTGCTGCTGGGGGACGTGGCTACA |
| | | CTGCCACTGCGCAAGGTGCTGGCTGTGGTGGGCACAGCGGAGGCC |
| | | TCCTCAGAACACCCATTAGGCGTAGCTGTCACAAAATACTGTAAG |
| | | GAGGAGCTGGGGACAGAGACCCTCGGCTATTGTACAGACTTCCAG |
| | | GCCGTGCCTGGCTGCGGCATCGGCTGTAAGGTCAGCAATGTGGAG |
| | | GGCATCCTGGCACACAGCGAGAGACCCCTCTCTGCCCCAGCCTCCC |
| | | ACCTGAATGAAGCTGGGTCTCTGCCTGCTGAGAAGGACGCCGTCC |
| | | CTCAGACCTTCTCCGTGTTGATTGGGAACAGAGAGTGGCTTAGAAG |
| | | GAATGGCCTGACCATCAGCTCTGACGTCTCGGATGCCATGACAGAT |
| | | CACGAGATGAAAGGCCAGACAGCCATACTGGTTGCCATCGACGGC |
| | | GTGCTCTGTGGCATGATTGCCATCGCTGATGCTGTTAAACAGGAAG |
| | | CTGCGCTGGCAGTCCACACCCTCCAGTCCATGGGAGTGGACGTGGT |
| | | CCTGATTACTGGAGACAACAGGAAAACCGCTCGGGCCATCGCTAC |
| | | CCAAGTGGGGATAAATAAGGTTTTTGCAGAAGTCTTACCCTCCCAC |
| | | AAAGTAGCCAAAGTGCAGGAGCTCCAGAACAAGGGTAAAAAGGT |
| | | GGCAATGGTGGGAGATGGTGTGAATGACAGCCCAGCTCTCGCGCA |
| | | GGCTGACATGGGCGTAGCCATAGGTACTGGAACAGATGTCGCGAT |
| | | TGAAGCCGCTGACGTAGTCCTCATCCGAAACGATTTGCTGGATGTG |
| | | GTGGCCAGCATCCATCTGTCCAAGCGTACCGTGCGCCGGATCCGCA |
| | | TCAACCTGGTCTTGGCCCTCATCTATAATCTCGTGGGCATCCCCAT |
| | | CGCTGCTGGGGTCTTCATGCCCATCGGCATTGTGCTGCAGCCCTGG |
| | | ATGGGAAGCGCTGCCATGGCTGCTTCTTCTGTCTCCGTTGTGCTGT |
| | | CCAGCCTACAGCTGAAGTGCTATAAAAAGCCAGACCTCGAGCGCT |
| | | ACGAAGCCCAGGCGCATGGCCATATGAAGCCCCTCACGGCTTCCC |
| | | AGGTCTCGGTTCACATTGGGATGGATGACAGATGGCGGGACTCCC |
| | | CACGCGCCACACCCTGGGACCAGGTATCCTATGTGAGCCAGGTGA |
| | | GCCTCAGCTCCCTGACATCAGATAAACCGTCTAGGCACTCAGCTGC |
| | | CGCAGATGATGACGGTGACAAATGGAGCCTCCTCCTGAACGGCCG |
| | | CGACGAGGAGCAGTACATCTAAGAATTCGATATCAAGCTTATCGA |
| | | TAATCAACCTCTGGATTACAAAATTTGTGAAAGATTGACTGGTATT |
| | | CTTAACTATGTTGCTCCTTTTACGCTATGTGGATACGCTGCTTTAAT |
| | | GCCTTTGTATCATGCTATTGCTTCCCGTATGGCTTTCATTTTCTCCT |
| | | CCTTGTATAAATCCTGGTTGCTGTCTCTTTATGAGGAGTTGTGGCCC |
| | | GTTGTCAGGCAACGTGGCGTGGTGTGCACTGTGTTTGCTGACGCAA |
| | | CCCCCACTGGTTGGGGCACCTGCAGGAATAAAAGATCTTTATTTTC |
| | | ATTAGATCTGTGTGTTGGTTTTTTGTGTGTCTAGAGTAGATAAGTA |
| | | GCATGGCGGGTTAATCATTAACTACAAGGAACCCCTAGTGATGGA |
| | | GTTGGCCACTCCCTCTCTGCGCGCTCGCTCGCTCACTGAGGCCGGG |
| | | CGACCAAAGGTCGCCCGACGCCCGGGCTTTGCCCGGGCGGCCTCA |
| | | GTGAGCGAGCGAGCGCGCAGAGAGGGAGTGGCCAA |
| 93 | A40 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGAGTTAATAATTACCAGCGCGGGCCAAAT |
| | | AAATAATCCGCGAGGGGCAGCTGGACTTTGGACTCCGCGCGCTGG |
| | | TAATTATTAACCTCGCGAATATTGATTCGAGGCCGCGATTGCCGCA |
| | | ATCGCGAGGGGCAGGTGACCTTTGCCCAGCGCGCGTTCGCCCCGC |
| | | CCCGGACGGTATCGATAAGCTTAGGAGCTTGGGCTGCAGGTCGAG |
| | | GGCACTGGGAGGATGTTGAGTAAGATGGAAAACTACTGATGACCC |
| | | TTGCAGAGACAGAGTATTAGGACATGTTTGAACAGGGGCCGGGCG |
| | | ATCAGCAGGTAGCTCTAGAGGATCCCCGTCTGTCTGCACATTTCGT |
| | | AGAGCGAGTGTTCCGATACTCTAATCTCCCTAGGCAAGGTTCATAT |
| | | TTGTGTAGGTTACTTATTCTCCTTTTGTTGACTAAGTCAATAATCAG |
| | | AATCAGCAGGTTTGGAGTCAGCTTGGCAGGGATCAGCAGCCTGGG |
| | | TTGGAAGGAGGGGGTATAAAAGCCCCTTCACCAGGAGAAGCCGTC |
| | | ACACAGATCCACAAGCTCGAGCCGAGTGGTCGTGCCTCCATAGAA |
| | | GCGGCCGCCACCATGCCGGAACAAGAACGCCAGATCACAGCAAGA |
| | | GAAGGAGCAAGTAGGAAGATCCTGAGCAAGCTCTCCCTGCCCACA |
| | | AGGGCCTGGGAGCCGGCCATGAAGAAGTCTTTTGCCTTCGATAAT |
| | | GTCGGTTATGAAGGCGGGCTCGATGGCCTGGGCCCCAGCAGCCAG |
| | | CCCCAGAAGTGTTTCCTGCAGATCAAGGGCATGACCTGTGCCAGCT |
| | | GTGTTAGCAATATCGAAAGAAACCTCCAAAAAGAGGCTGGAGTTC |
| | | TGAGTGTTCTAGTGGCTTTAATGGCCGGGAAAGCCGAGATCAAGT |
| | | ATGATCCCGAGGTGATTCAGCCATTGGAGATTGCCCAGTTCATCCA |
| | | GGATCTGGGCTTCGAGGCCGCCGTGATGGAGGATTACGCCGGCTC |
| | | TGATGGCAACATCGAACTGACCATCACCGGCATGACCTGTGCTTCC |
| | | TGCGTGCATAATATCGAGAGCAAACTGACGCGGACCAATGGAATC |
| | | ACCTATGCCAGCGTCGCCCTGGCAACCTCTAAGGCTCTGGTGAAGT |
| | | TTGACCCCGAAATCATTGGACCTAGAGACATCATCAAAATTATTGA |
| | | AGAGATTGGTTTCCACGCCAGCTTAGCTCAGCGGAATCCCAACGC |
| | | ACACCATCTTGACCATAAGATGGAAATCAAACAGTGGAAGAAGAG |
| | | CTTCTTGTGTAGCCTGGTCTTCGGGATACCCGTCATGGCACTGATG |
| | | ATTTACATGCTCATTCCGTCCAACGAGCCCCACCAGTCCATGGTGC |
| | | TGGACCACAACATCATCCCTGGGCTGTCTATCCTCAACCTGATCTT |
| | | CTTTATTCTGTGCACATTTGTCCAGCTCTTGGGAGGCTGGTATTTCT |
| | | ATGTACAGGCCTACAAATCCCTGAGGCATCGAAGCGCCAACATGG |
| | | ACGTTTTAATTGTGCTGGCTACATCCATTGCCTACGTGTACTCCCTG |
| | | GTGATCTTAGTGGTTGCTGTGGCTGAAAAGGCTGAGAGAAGCCCT |
| | | GTCACCTTCTTCGACACCCCCCCCATGCTCTTCGTGTTTATTGCACT |
| | | GGGTCGCTGGCTGGAGCATTTAGCCAAGTCCAAGACATCCGAGGC |
| | | TCTGGCCAAGCTGATGTCTCTACAGGCCACGGAGGCCACCGTGGT |
| | | GACTCTGGGTGAAGACAATTTGATAATACGCGAGGAACAGGTGCC |
| | | TATGGAGCTTGTGCAGCGCGGAGATATCGTCAAGGTGGTGCCCGG |
| | | CGGCAAGTTCCCAGTGGATGGAAAAGTGTTAGAGGGCAATACCAT |
| | | GGCCGATGAATCCCTCATCACTGGCGAGGCAATGCCGGTGACCAA |
| | | GAAACCTGGAAGTACTGTCATTGCAGGCAGCATCAATGCCCATGG |
| | | GAGCGTCCTGATCAAGGCCACCCACGTGGGCAATGATACAACTCT |
| | | CGCCCAGATCGTCAAGCTAGTGGAAGAGGCTCAGATGTCTAAAGC |
| | | TCCAATCCAGCAACTGGCTGACCGCTTCAGCGGCTACTTTGTCCCC |
| | | TTTATCATCATCATGAGCACCCTCACCCTGGTGGTGTGGATAGTCA |
| | | TAGGCTTCATTGACTTTGGCGTGGTGCAGAGATACTTCCCCAACCC |
| | | TAACAAACATATCTCCCAAACCGAGGTCATTATCAGATTCGCCTTT |
| | | CAGACATCTATCACTGTGCTGTGCATAGCCTGCCCTTGCTCTCTGG |
| | | GCCTGGCCACCCCCACCGCCGTGATGGTTGGAACCGGGGTCGCTG |
| | | CACAAAATGGGATCCTGATAAAAGGGGGCAAACCCCTGGAAATGG |
| | | CACATAAGATTAAAACAGTTATGTTTGATAAGACAGGCACCATTA |
| | | CTCACGGTGTCCCTCGAGTCATGAGAGTGCTGCTGCTGGGGGACGT |
| | | GGCTACACTGCCACTGCGCAAGGTGCTGGCTGTGGTGGGCACAGC |
| | | GGAGGCCTCCTCAGAACACCCATTAGGCGTAGCTGTCACAAAATA |
| | | CTGTAAGGAGGAGCTGGGGACAGAGACCCTCGGCTATTGTACAGA |
| | | CTTCCAGGCCGTGCCTGGCTGCGGCATCGGCTGTAAGGTCAGCAAT |
| | | GTGGAGGGCATCCTGGCACACAGCGAGAGACCCCTCTCTGCCCCA |
| | | GCCTCCCACCTGAATGAAGCTGGGTCTCTGCCTGCTGAGAAGGAC |
| | | GCCGTCCCTCAGACCTTCTCCGTGTTGATTGGGAACAGAGAGTGGC |
| | | TTAGAAGGAATGGCCTGACCATCAGCTCTGACGTCTCGGATGCCAT |
| | | GACAGATCACGAGATGAAAGGCCAGACAGCCATACTGGTTGCCAT |
| | | CGACGGCGTGCTCTGTGGCATGATTGCCATCGCTGATGCTGTTAAA |
| | | CAGGAAGCTGCGCTGGCAGTCCACACCCTCCAGTCCATGGGAGTG |
| | | GACGTGGTCCTGATTACTGGAGACAACAGGAAAACCGCTCGGGCC |
| | | ATCGCTACCCAAGTGGGGATAAATAAGGTTTTTGCAGAAGTCTTAC |
| | | CCTCCCACAAAGTAGCCAAAGTGCAGGAGCTCCAGAACAAGGGTA |
| | | AAAAGGTGGCAATGGTGGGAGATGGTGTGAATGACAGCCCAGCTC |
| | | TCGCGCAGGCTGACATGGGCGTAGCCATAGGTACTGGAACAGATG |
| | | TCGCGATTGAAGCCGCTGACGTAGTCCTCATCCGAAACGATTTGCT |
| | | GGATGTGGTGGCCAGCATCCATCTGTCCAAGCGTACCGTGCGCCG |
| | | GATCCGCATCAACCTGGTCTTGGCCCTCATCTATAATCTCGTGGGC |
| | | ATCCCCATCGCTGCTGGGGTCTTCATGCCCATCGGCATTGTGCTGC |
| | | AGCCCTGGATGGGAAGCGCTGCCATGGCTGCTTCTTCTGTCTCCGT |
| | | TGTGCTGTCCAGCCTACAGCTGAAGTGCTATAAAAAGCCAGACCTC |
| | | GAGCGCTACGAAGCCCAGGCGCATGGCCATATGAAGCCCCTCACG |
| | | GCTTCCCAGGTCTCGGTTCACATTGGGATGGATGACAGATGGCGG |
| | | GACTCCCCACGCGCCACACCCTGGGACCAGGTATCCTATGTGAGCC |
| | | AGGTGAGCCTCAGCTCCCTGACATCAGATAAACCGTCTAGGCACTC |
| | | AGCTGCCGCAGATGATGACGGTGACAAATGGAGCCTCCTCCTGAA |
| | | CGGCCGCGACGAGGAGCAGTACATCTAAGAATTCGATATCAAGCT |
| | | TATCGATAATCAACCTCTGGATTACAAAATTTGTGAAAGATTGACT |
| | | GGTATTCTTAACTATGTTGCTCCTTTTACGCTATGTGGATACGCTGC |
| | | TTTAATGCCTTTGTATCATGCTATTGCTTCCCGTATGGCTTTCATTT |
| | | TCTCCTCCTTGTATAAATCCTGGTTGCTGTCTCTTTATGAGGAGTTG |
| | | TGGCCCGTTGTCAGGCAACGTGGCGTGGTGTGCACTGTGTTTGCTG |
| | | ACGCAACCCCCACTGGTTGGGGCACCTGCAGGCAGACATGATAAG |
| | | ATACATTGATGAGTTTGGACAAACCACAACTAGAATGCAGTGAAA |
| | | AAAATGCTTTATTTGTGAAATTTGTGATGCTATTGCTTTATTTGTAA |
| | | CCATTATAAGCTGCAATAAACAAGTTAACAACAACAATTGCATTC |
| | | ATTTTATGTTTCAGGTTCAGGGGGAGGTGTGGGAGGTTTTTTAAAG |
| | | CAAGTAAAACCTCTACAAATGTGGTATCTAGAGTAGATAAGTAGC |
| | | ATGGCGGGTTAATCATTAACTACAAGGAACCCCTAGTGATGGAGT |
| | | TGGCCACTCCCTCTCTGCGCGCTCGCTCGCTCACTGAGGCCGGGCG |
| | | ACCAAAGGTCGCCCGACGCCCGGGCTTTGCCCGGGCGGCCTCAGT |
| | | GAGCGAGCGAGCGCGCAGAGAGGGAGTGGCCAA |
| 94 | A41 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGATCCCATAAAACTCTAGCCTCCCAGAGGGA |
| | | CCCCAGCCCCCACCCTCCCGCCCACGAACCCCTGCATTTCCAGAAT |
| | | CAGCCCCAGGGCCCCAACCCCCCCAAGCCCCCATTTCACAACACG |
| | | CTGGCGCTACAGGCGCGTGACTTCCCCTTGCTTTGGGGCGGGGGGC |
| | | TGAGACTCCTATGTGCTCCGGATTGGTCAGGCACGGCCTTCGGCCC |
| | | CGCCTCCTGCCACCGCAGATTGGCCGCTAGCCCTCCCCGAGCGCCC |
| | | TGCCTCCGAGGGCCGGCGCACTATAAAAGCCCCTTCACCAGGAGA |
| | | AGCCGTCACACAGATCCACAAGCTCGAGCCGAGTGGTCGTGGGTC |
| | | TGTCTCTTGCTTCAACAGTGTTTGGACGGAACAGATCCGGGGACTC |
| | | TCTTCCAGCCTCCGACCGCCCTCCGATTTCCTCTCCGCTTGCAACCT |
| | | CCGGGACCATCTTCTCGGCCATCTCCTGCTTCTGGGACCTGCCAGC |
| | | ACCGTTTTTGTGGTTAGCTCCTTCTTGCCAACCAACCGGATCCGCC |
| | | ACCATGCCGGAACAAGAACGCCAGATCACAGCAAGAGAAGGAGC |
| | | AAGTAGGAAGATCCTGAGCAAGCTCTCCCTGCCCACAAGGGCCTG |
| | | GGAGCCGGCCATGAAGAAGTCTTTTGCCTTCGATAATGTCGGTTAT |
| | | GAAGGCGGGCTCGATGGCCTGGGCCCCAGCAGCCAGCCCCAGAAG |
| | | TGTTTCCTGCAGATCAAGGGCATGACCTGTGCCAGCTGTGTTAGCA |
| | | ATATCGAAAGAAACCTCCAAAAAGAGGCTGGAGTTCTGAGTGTTC |
| | | TAGTGGCTTTAATGGCCGGGAAAGCCGAGATCAAGTATGATCCCG |
| | | AGGTGATTCAGCCATTGGAGATTGCCCAGTTCATCCAGGATCTGGG |
| | | CTTCGAGGCCGCCGTGATGGAGGATTACGCCGGCTCTGATGGCAA |
| | | CATCGAACTGACCATCACCGGCATGACCTGTGCTTCCTGCGTGCAT |
| | | AATATCGAGAGCAAACTGACGCGGACCAATGGAATCACCTATGCC |
| | | AGCGTCGCCCTGGCAACCTCTAAGGCTCTGGTGAAGTTTGACCCCG |
| | | AAATCATTGGACCTAGAGACATCATCAAAATTATTGAAGAGATTG |
| | | GTTTCCACGCCAGCTTAGCTCAGCGGAATCCCAACGCACACCATCT |
| | | TGACCATAAGATGGAAATCAAACAGTGGAAGAAGAGCTTCTTGTG |
| | | TAGCCTGGTCTTCGGGATACCCGTCATGGCACTGATGATTTACATG |
| | | CTCATTCCGTCCAACGAGCCCCACCAGTCCATGGTGCTGGACCACA |
| | | ACATCATCCCTGGGCTGTCTATCCTCAACCTGATCTTCTTTATTCTG |
| | | TGCACATTTGTCCAGCTCTTGGGAGGCTGGTATTTCTATGTACAGG |
| | | CCTACAAATCCCTGAGGCATCGAAGCGCCAACATGGACGTTTTAAT |
| | | TGTGCTGGCTACATCCATTGCCTACGTGTACTCCCTGGTGATCTTA |
| | | GTGGTTGCTGTGGCTGAAAAGGCTGAGAGAAGCCCTGTCACCTTCT |
| | | TCGACACCCCCCCCATGCTCTTCGTGTTTATTGCACTGGGTCGCTG |
| | | GCTGGAGCATTTAGCCAAGTCCAAGACATCCGAGGCTCTGGCCAA |
| | | GCTGATGTCTCTACAGGCCACGGAGGCCACCGTGGTGACTCTGGGT |
| | | GAAGACAATTTGATAATACGCGAGGAACAGGTGCCTATGGAGCTT |
| | | GTGCAGCGCGGAGATATCGTCAAGGTGGTGCCCGGCGGCAAGTTC |
| | | CCAGTGGATGGAAAAGTGTTAGAGGGCAATACCATGGCCGATGAA |
| | | TCCCTCATCACTGGCGAGGCAATGCCGGTGACCAAGAAACCTGGA |
| | | AGTACTGTCATTGCAGGCAGCATCAATGCCCATGGGAGCGTCCTG |
| | | ATCAAGGCCACCCACGTGGGCAATGATACAACTCTCGCCCAGATC |
| | | GTCAAGCTAGTGGAAGAGGCTCAGATGTCTAAAGCTCCAATCCAG |
| | | CAACTGGCTGACCGCTTCAGCGGCTACTTTGTCCCCTTTATCATCAT |
| | | CATGAGCACCCTCACCCTGGTGGTGTGGATAGTCATAGGCTTCATT |
| | | GACTTTGGCGTGGTGCAGAGATACTTCCCCAACCCTAACAAACATA |
| | | TCTCCCAAACCGAGGTCATTATCAGATTCGCCTTTCAGACATCTAT |
| | | CACTGTGCTGTGCATAGCCTGCCCTTGCTCTCTGGGCCTGGCCACC |
| | | CCCACCGCCGTGATGGTTGGAACCGGGGTCGCTGCACAAAATGGG |
| | | ATCCTGATAAAAGGGGGCAAACCCCTGGAAATGGCACATAAGATT |
| | | AAAACAGTTATGTTTGATAAGACAGGCACCATTACTCACGGTGTCC |
| | | CTCGAGTCATGAGAGTGCTGCTGCTGGGGGACGTGGCTACACTGC |
| | | CACTGCGCAAGGTGCTGGCTGTGGTGGGCACAGCGGAGGCCTCCT |
| | | CAGAACACCCATTAGGCGTAGCTGTCACAAAATACTGTAAGGAGG |
| | | AGCTGGGGACAGAGACCCTCGGCTATTGTACAGACTTCCAGGCCG |
| | | TGCCTGGCTGCGGCATCGGCTGTAAGGTCAGCAATGTGGAGGGCA |
| | | TCCTGGCACACAGCGAGAGACCCCTCTCTGCCCCAGCCTCCCACCT |
| | | GAATGAAGCTGGGTCTCTGCCTGCTGAGAAGGACGCCGTCCCTCA |
| | | GACCTTCTCCGTGTTGATTGGGAACAGAGAGTGGCTTAGAAGGAA |
| | | TGGCCTGACCATCAGCTCTGACGTCTCGGATGCCATGACAGATCAC |
| | | GAGATGAAAGGCCAGACAGCCATACTGGTTGCCATCGACGGCGTG |
| | | CTCTGTGGCATGATTGCCATCGCTGATGCTGTTAAACAGGAAGCTG |
| | | CGCTGGCAGTCCACACCCTCCAGTCCATGGGAGTGGACGTGGTCCT |
| | | GATTACTGGAGACAACAGGAAAACCGCTCGGGCCATCGCTACCCA |
| | | AGTGGGGATAAATAAGGTTTTTGCAGAAGTCTTACCCTCCCACAAA |
| | | GTAGCCAAAGTGCAGGAGCTCCAGAACAAGGGTAAAAAGGTGGC |
| | | AATGGTGGGAGATGGTGTGAATGACAGCCCAGCTCTCGCGCAGGC |
| | | TGACATGGGCGTAGCCATAGGTACTGGAACAGATGTCGCGATTGA |
| | | AGCCGCTGACGTAGTCCTCATCCGAAACGATTTGCTGGATGTGGTG |
| | | GCCAGCATCCATCTGTCCAAGCGTACCGTGCGCCGGATCCGCATCA |
| | | ACCTGGTCTTGGCCCTCATCTATAATCTCGTGGGCATCCCCATCGC |
| | | TGCTGGGGTCTTCATGCCCATCGGCATTGTGCTGCAGCCCTGGATG |
| | | GGAAGCGCTGCCATGGCTGCTTCTTCTGTCTCCGTTGTGCTGTCCA |
| | | GCCTACAGCTGAAGTGCTATAAAAAGCCAGACCTCGAGCGCTACG |
| | | AAGCCCAGGCGCATGGCCATATGAAGCCCCTCACGGCTTCCCAGG |
| | | TCTCGGTTCACATTGGGATGGATGACAGATGGCGGGACTCCCCAC |
| | | GCGCCACACCCTGGGACCAGGTATCCTATGTGAGCCAGGTGAGCC |
| | | TCAGCTCCCTGACATCAGATAAACCGTCTAGGCACTCAGCTGCCGC |
| | | AGATGATGACGGTGACAAATGGAGCCTCCTCCTGAACGGCCGCGA |
| | | CGAGGAGCAGTACATCTAAGAATTCGATATCAAGCTTATCGATAA |
| | | TCAACCTCTGGATTACAAAATTTGTGAAAGATTGACTGGTATTCTT |
| | | AACTATGTTGCTCCTTTTACGCTATGTGGATACGCTGCTTTAATGCC |
| | | TTTGTATCATGCTATTGCTTCCCGTATGGCTTTCATTTTCTCCTCCTT |
| | | GTATAAATCCTGGTTGCTGTCTCTTTATGAGGAGTTGTGGCCCGTT |
| | | GTCAGGCAACGTGGCGTGGTGTGCACTGTGTTTGCTGACGCAACCC |
| | | CCACTGGTTGGGGCACCTGCAGGAATAAAAGATCTTTATTTTCATT |
| | | AGATCTGTGTGTTGGTTTTTTGTGTGTCTAGAGTAGATAAGTAGCA |
| | | TGGCGGGTTAATCATTAACTACAAGGAACCCCTAGTGATGGAGTT |
| | | GGCCACTCCCTCTCTGCGCGCTCGCTCGCTCACTGAGGCCGGGCGA |
| | | CCAAAGGTCGCCCGACGCCCGGGCTTTGCCCGGGCGGCCTCAGTG |
| | | AGCGAGCGAGCGCGCAGAGAGGGAGTGGCCAA |
| 95 | A42 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGATCCCATAAAACTCTAGCCTCCCAGAGGGA |
| | | CCCCAGCCCCCACCCTCCCGCCCACGAACCCCTGCATTTCCAGAAT |
| | | CAGCCCCAGGGCCCCAACCCCCCCAAGCCCCCATTTCACAACACG |
| | | CTGGCGCTACAGGCGCGTGACTTCCCCTTGCTTTGGGGCGGGGGGC |
| | | TGAGACTCCTATGTGCTCCGGATTGGTCAGGCACGGCCTTCGGCCC |
| | | CGCCTCCTGCCACCGCAGATTGGCCGCTAGCCCTCCCCGAGCGCCC |
| | | TGCCTCCGAGGGCCGGCGCACTATAAAAGCCCCTTCACCAGGAGA |
| | | AGCCGTCACACAGATCCACAAGCTCGAGCCGAGTGGTCGTGGGTC |
| | | TGTCTCTTGCTTCAACAGTGTTTGGACGGAACAGATCCGGGGACTC |
| | | TCTTCCAGCCTCCGACCGCCCTCCGATTTCCTCTCCGCTTGCAACCT |
| | | CCGGGACCATCTTCTCGGCCATCTCCTGCTTCTGGGACCTGCCAGC |
| | | ACCGTTTTTGTGGTTAGCTCCTTCTTGCCAACCAACCGGATCCGCC |
| | | ACCATGCCCGAACAGGAAAGACAGATTACCGCCCGCGAGGGCGCT |
| | | TCCCGCAAAATTCTGAGCAAACTGAGCCTGCCCACCCGCGCCTGG |
| | | GAACCTGCCATGAAGAAAAGCTTTGCTTTTGATAATGTCGGCTATG |
| | | AGGGCGGCCTGGACGGCCTGGGCCCTAGCAGCCAGGTGGCTACCA |
| | | CCCTGATTGCCATTGCCGGCATGACCTGCGCCAGCTGCGTGCATAG |
| | | CATTGAGGGCATGATTAGCCAGCTGGAGGGCGTGCAGCAGATTTC |
| | | CGTGAGCCTGGCTGAGGGCACCGCTACCGTGCTGTATAATCCCTCC |
| | | GTGATTAGCCCCGAAGAACTGCGCGCTGCCATTGAAGATATGGGC |
| | | TTTGAAGCCTCCGTGGTGTCCGAAAGCTGCAGCACCAATCCCCTGG |
| | | GCAATCATTCCGCCGGCAATAGCATGGTGCAGACCACCGACGGCA |
| | | CCCCCACCTCCGTGCAGGAAGTGGCTCCCCATACCGGCCGCCTGCC |
| | | CGCCAATCATGCCCCTGATATTCTGGCTAAAAGCCCCCAGAGCACC |
| | | CGCGCCGTGGCTCCTCAGAAATGCTTTCTGCAGATTAAGGGCATGA |
| | | CCTGCGCTAGCTGCGTGAGCAATATTGAACGCAATCTGCAGAAAG |
| | | AAGCCGGCGTGCTGTCCGTGCTGGTGGCCCTGATGGCCGGCAAAG |
| | | CTGAAATCAAATATGATCCCGAAGTGATTCAGCCCCTGGAAATTGC |
| | | CCAGTTCATTCAGGATCTGGGCTTTGAGGCTGCCGTGATGGAAGAT |
| | | TATGCCGGCTCCGACGGCAATATTGAACTGACCATTACCGGCATGA |
| | | CCTGCGCCAGCTGCGTGCATAATATTGAAAGCAAACTGACCCGCA |
| | | CCAACGGCATTACCTATGCTTCCGTGGCCCTGGCTACCAGCAAAGC |
| | | CCTGGTGAAATTTGATCCTGAAATTATCGGCCCCCGCGATATTATC |
| | | AAAATTATTGAAGAAATCGGCTTTCACGCCAGCCTGGCTCAGCGC |
| | | AATCCCAATGCCCATCATCTGGATCATAAGATGGAAATCAAACAG |
| | | TGGAAGAAAAGCTTTCTGTGCAGCCTGGTGTTCGGCATTCCTGTGA |
| | | TGGCCCTGATGATCTATATGCTGATTCCCAGCAATGAACCCCATCA |
| | | GAGCATGGTGCTGGATCATAATATTATTCCCGGCCTGAGCATTCTG |
| | | AATCTGATTTTCTTTATCCTGTGCACCTTTGTGCAGCTGCTGGGCGG |
| | | CTGGTATTTCTATGTGCAGGCTTATAAAAGCCTGCGCCACCGCTCC |
| | | GCCAATATGGATGTGCTGATTGTGCTGGCCACCAGCATTGCTTATG |
| | | TCTATAGCCTGGTCATTCTGGTGGTGGCCGTGGCCGAGAAAGCTGA |
| | | ACGCAGCCCCGTGACCTTCTTTGATACCCCTCCCATGCTGTTCGTGT |
| | | TTATCGCTCTGGGCCGCTGGCTGGAACATCTGGCCAAAAGCAAAA |
| | | CCTCCGAAGCCCTGGCTAAACTGATGAGCCTGCAGGCCACCGAAG |
| | | CCACCGTGGTGACCCTGGGCGAAGATAATCTGATTATCCGCGAAG |
| | | AACAGGTGCCTATGGAACTGGTGCAGCGCGGCGATATTGTGAAAG |
| | | TGGTGCCCGGCGGCAAATTTCCCGTGGACGGCAAAGTGCTGGAGG |
| | | GCAATACCATGGCTGATGAATCCCTGATTACCGGCGAAGCTATGCC |
| | | TGTGACCAAGAAACCCGGCAGCACCGTGATTGCCGGCAGCATTAA |
| | | TGCTCACGGCTCCGTGCTGATCAAGGCTACCCATGTCGGCAATGAT |
| | | ACCACCCTGGCCCAGATTGTGAAACTGGTGGAAGAAGCCCAGATG |
| | | AGCAAAGCCCCTATTCAGCAGCTGGCTGACCGCTTTTCCGGCTATT |
| | | TTGTGCCCTTTATCATTATTATGAGCACCCTGACCCTGGTGGTGTG |
| | | GATTGTGATCGGCTTCATTGATTTCGGCGTGGTGCAGAGATATTTT |
| | | CCTAATCCCAACAAACATATTAGCCAGACCGAAGTGATTATCCGCT |
| | | TTGCTTTTCAGACCAGCATTACCGTGCTGTGCATTGCTTGCCCCTGC |
| | | AGCCTGGGCCTGGCTACCCCCACCGCCGTCATGGTCGGCACCGGC |
| | | GTGGCTGCCCAGAACGGCATTCTGATTAAGGGCGGCAAACCTCTG |
| | | GAAATGGCCCACAAAATCAAAACCGTGATGTTTGATAAAACCGGC |
| | | ACCATTACCCACGGCGTGCCCCGCGTGATGCGCGTGCTGCTGCTGG |
| | | GCGATGTGGCTACCCTGCCCCTGCGCAAAGTGCTGGCCGTGGTCGG |
| | | CACCGCCGAAGCTAGCTCCGAACATCCCCTGGGCGTGGCCGTGAC |
| | | CAAATATTGCAAAGAAGAACTGGGCACCGAAACCCTGGGCTATTG |
| | | CACCGATTTTCAGGCCGTCCCTGGCTGCGGCATCGGCTGCAAAGTG |
| | | AGCAATGTGGAGGGCATTCTGGCCCATTCCGAAAGACCCCTGTCC |
| | | GCCCCTGCTAGCCATCTGAATGAAGCCGGCAGCCTGCCTGCTGAG |
| | | AAAGATGCCGTGCCCCAGACCTTTTCCGTGCTGATCGGCAACCGCG |
| | | AATGGCTGCGCCGCAACGGCCTGACCATTAGCTCCGATGTCTCCGA |
| | | TGCCATGACCGATCATGAAATGAAAGGCCAGACCGCCATTCTGGT |
| | | GGCCATTGACGGCGTGCTGTGCGGCATGATTGCCATTGCCGATGCC |
| | | GTGAAACAGGAGGCTGCCCTGGCTGTGCATACCCTGCAGAGCATG |
| | | GGCGTGGATGTGGTGCTGATTACCGGCGATAACCGCAAAACCGCT |
| | | CGCGCCATTGCCACCCAGGTCGGCATTAACAAAGTGTTTGCTGAAG |
| | | TGCTGCCCAGCCATAAAGTGGCTAAAGTGCAGGAACTGCAGAACA |
| | | AAGGCAAGAAAGTGGCTATGGTCGGCGACGGCGTGAATGATAGCC |
| | | CTGCCCTGGCCCAGGCCGATATGGGCGTGGCTATCGGCACCGGCA |
| | | CCGATGTGGCTATTGAGGCTGCCGATGTGGTGCTGATCCGCAATGA |
| | | TCTGCTGGATGTGGTGGCTAGCATTCATCTGAGCAAACGCACCGTC |
| | | CGCCGCATCCGCATTAATCTGGTGCTGGCCCTGATCTATAATCTGG |
| | | TCGGCATTCCCATCGCCGCCGGCGTGTTTATGCCCATCGGCATTGT |
| | | GCTGCAGCCCTGGATGGGCAGCGCCGCTATGGCCGCTAGCTCCGT |
| | | GTCCGTGGTGCTGAGCAGCCTGCAGCTGAAATGCTATAAGAAACC |
| | | CGATCTGGAAAGATATGAAGCTCAGGCTCACGGCCATATGAAACC |
| | | CCTGACCGCCAGCCAGGTGTCCGTGCATATCGGCATGGATGACCG |
| | | CTGGCGCGATAGCCCCCGCGCCACCCCTTGGGATCAGGTGAGCTAT |
| | | GTGAGCCAGGTGTCCCTGAGCTCCCTGACCTCCGATAAACCCTCCC |
| | | GCCATTCCGCCGCTGCCGATGATGACGGCGATAAGTGGAGCCTGC |
| | | TGCTGAACGGCCGCGATGAAGAACAGTATATCTAAGTCCATAAAG |
| | | TAGGAAACACTACATCATCCATAAAGTAGGAAACACTACATCATC |
| | | CATAAAGTAGGAAACACTACATCATCCATAAAGTAGGAAACACTA |
| | | CAGAATTCGATATCAAGCTTATCGATAATCAACCTCTGGATTACAA |
| | | AATTTGTGAAAGATTGACTGGTATTCTTAACTATGTTGCTCCTTTTA |
| | | CGCTATGTGGATACGCTGCTTTAATGCCTTTGTATCATGCTATTGCT |
| | | TCCCGTATGGCTTTCATTTTCTCCTCCTTGTATAAATCCTGGTTGCT |
| | | GTCTCTTTATGAGGAGTTGTGGCCCGTTGTCAGGCAACGTGGCGTG |
| | | GTGTGCACTGTGTTTGCTGACGCAACCCCCACTGGTTGGGGCACCT |
| | | GCAGGAATAAAAGATCTTTATTTTCATTAGATCTGTGTGTTGGTTTT |
| | | TTGTGTGTCTAGAGTAGATAAGTAGCATGGCGGGTTAATCATTAAC |
| | | TACAAGGAACCCCTAGTGATGGAGTTGGCCACTCCCTCTCTGCGCG |
| | | CTCGCTCGCTCACTGAGGCCGGGCGACCAAAGGTCGCCCGACGCC |
| | | CGGGCTTTGCCCGGGCGGCCTCAGTGAGCGAGCGAGCGCGCAGAG |
| | | AGGGAGTGGCCAA |
| 96 | A43 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGATCCCATAAAACTCTAGCCTCCCAGAGGGA |
| | | CCCCAGCCCCCACCCTCCCGCCCACGAACCCCTGCATTTCCAGAAT |
| | | CAGCCCCAGGGCCCCAACCCCCCCAAGCCCCCATTTCACAACACG |
| | | CTGGCGCTACAGGCGCGTGACTTCCCCTTGCTTTGGGGCGGGGGGC |
| | | TGAGACTCCTATGTGCTCCGGATTGGTCAGGCACGGCCTTCGGCCC |
| | | CGCCTCCTGCCACCGCAGATTGGCCGCTAGCCCTCCCCGAGCGCCC |
| | | TGCCTCCGAGGGCCGGCGCACTATAAAAGCCCCTTCACCAGGAGA |
| | | AGCCGTCACACAGATCCACAAGCTCGAGCCGAGTGGTCGTGGGTC |
| | | TGTCTCTTGCTTCAACAGTGTTTGGACGGAACAGATCCGGGGACTC |
| | | TCTTCCAGCCTCCGACCGCCCTCCGATTTCCTCTCCGCTTGCAACCT |
| | | CCGGGACCATCTTCTCGGCCATCTCCTGCTTCTGGGACCTGCCAGC |
| | | ACCGTTTTTGTGGTTAGCTCCTTCTTGCCAACCAACCGGATCCGCC |
| | | ACCATGGCGGCGCCTGAGCAGGAGAGACAGATCACAGCCAGAGA |
| | | AGGGGCCAGTCGGAAAATCTTATCTAAGCTTTCTTTGCCTACCCGT |
| | | GCCTGGGAACCAGCAATGAAGAAGAGTTTTGCTTTTGACAATGTTG |
| | | GCTATGAAGGTGGTCTGGATGGCCTGGGCCCTTCTTCTCAGGTGGC |
| | | CCCTGATGGAGCCGAAGGGAGTGGGACAGATCACAGGTCTTCCAG |
| | | TTCTCATTCCCCTGGCTCCCCACCGAGAAACCAGGTCCAGGGCACA |
| | | TGCAGTAAGTGCTTCTTACAGATCAAAGGCATGACCTGTGCATCCT |
| | | GTGTGTCTAACATAGAAAGGAATCTGCAGAAAGAAGCTGGTGTTC |
| | | TCTCCGTGTTGGTTGCCTTGATGGCAGGAAAGGCAGAGATCAAGT |
| | | ATGACCCAGAGGTCATCCAGCCCCTCGAGATAGCTCAGTTCATCCA |
| | | GGACCTGGGTTTTGAGGCAGCAGTCATGGAGGACTACGCAGGCTC |
| | | CGATGGCAACATTGAGCTGACAATCACAGGGATGACCTGCGCGTC |
| | | CTGTGTCCACAACATAGAGTCCAAACTCACGAGGACAAATGGCAT |
| | | CACTTATGCCTCCGTTGCCCTTGCCACCAGCAAAGCCCTTGTTAAG |
| | | TTTGACCCGGAAATTATCGGTCCACGGGATATTATCAAAATTATTG |
| | | AGGAAATTGGCTTTCATGCTTCCCTGGCCCAGAGAAACCCCAACGC |
| | | TCATCACTTGGACCACAAGATGGAAATAAAGCAGTGGAAGAAGTC |
| | | TTTTCTTGTGAGTCTGTTTTTCTGTATTCCTGTAATGGGGCTGATGA |
| | | TATATATGATGGTTATGGACCACCACTTTGCAACTCTTCACCATAA |
| | | TCAAAACATGAGTAAAGAAGAAATGATCAACCTTCATTCTTCTATG |
| | | TTCCTGGAGCGCCAGATTCTTCCAGGATTGTCTGTTATGAATTTGCT |
| | | GTCCTTTTTATTGTGTGTACCTGTACAGTTTTTCGGAGGCTGGTACT |
| | | TCTACATTCAGGCTTATAAAGCACTGAAGCATAAGACAGCAAATA |
| | | TGGACGTACTGATTGTGCTGGCAACCACCATTGCATTTGCCTACTC |
| | | TTTGATTATTCTTCTAGTTGCAATGTATGAGAGAGCCAAAGTGAAC |
| | | CCTATTACTTTCTTTGACACACCCCCTATGCTGTTTGTGTTTATTGC |
| | | ACTAGGCCGATGGCTGGAACATATAGCAAAGGGCAAAACATCAGA |
| | | GGCTCTTGCAAAGTTAATTTCACTACAAGCTACAGAAGCAACTATT |
| | | GTAACTCTTGATTCTGATAATATCCTCCTCAGTGAAGAACAAGTGG |
| | | ATGTGGAACTTGTACAACGTGGAGATATCATTAAAGTAGTTCCAG |
| | | GAGGCAAATTTCCAGTGGATGGTCGTGTTATTGAAGGACATTCTAT |
| | | GGTAGATGAGTCCCTCATCACAGGGGAGGCAATGCCTGTGGCTAA |
| | | GAAACCTGGCAGCACAGTGATTGCTGGTTCCATTAACCAGAACGG |
| | | GTCACTGCTTATCTGCGCAACACATGTTGGAGCAGACACAACCCTT |
| | | TCTCAAATTGTCAAACTTGTGGAAGAGGCACAAACATCAAAGGCT |
| | | CCTATCCAGCAGTTTGCAGACAAACTCAGTGGCTATTTTGTTCCTTT |
| | | TATTGTTTTTGTTTCCATTGCCACCCTCTTGGTATGGATTGTAATTG |
| | | GATTTCTGAATTTTGAAATTGTGGAAACCTACTTTCCTGGCTACAA |
| | | TAGAAGTATCTCCCGAACAGAAACGATAATACGATTTGCTTTCCAA |
| | | GCCTCTATCACAGTTCTGTGTATTGCATGTCCCTGTTCACTGGGACT |
| | | GGCCACTCCAACTGCTGTGATGGTGGGTACAGGAGTAGGTGCTCA |
| | | AAATGGCATACTAATAAAAGGTGGAGAGCCATTGGAGATGGCTCA |
| | | TAAGGTAAAGGTAGTGGTATTTGATAAGACTGGAACCATTACTCA |
| | | CGGAACCCCAGTGGTGAATCAAGTAAAGGTTCTAACTGAAAGTAA |
| | | CAGAATATCACACCATAAAATCTTGGCCATTGTGGGAACTGCTGA |
| | | AAGTAACAGTGAACACCCTCTAGGAACAGCCATAACCAAATATTG |
| | | CAAACAGGAGCTGGACACTGAAACCTTGGGTACCTGCATAGATTT |
| | | CCAGGTTGTGCCAGGCTGTGGTATTAGCTGTAAAGTCACCAATATT |
| | | GAAGGCTTGCTACATAAGAATAACTGGAATATAGAGGACAATAAT |
| | | ATTAAAAATGCATCCCTGGTTCAAATTGATGCCAGTAATGAACAGT |
| | | CATCAACTTCGTCTTCCATGATTATTGATGCCCAGATCTCAAATGC |
| | | TCTTAATGCTCAGCAGTATAAAGTCCTCATTGGTAACCGGGAGTGG |
| | | ATGATTAGAAATGGTCTTGTCATTAATAACGATGTAAATGATTTCA |
| | | TGACTGAACATGAGAGAAAAGGTCGGACTGCTGTATTAGTAGCAG |
| | | TTGATGATGAGCTGTGTGGCTTGATAGCCATTGCAGACACAGTGAA |
| | | GCCTGAAGCAGAACTGGCTATCCATATTCTGAAATCTATGGGCTTA |
| | | GAAGTAGTTCTGATGACTGGAGACAACAGTAAAACAGCTAGATCT |
| | | ATTGCTTCTCAGGTTGGCATTACTAAGGTGTTTGCTGAAGTTCTAC |
| | | CTTCTCACAAGGTTGCTAAAGTGAAGCAACTTCAAGAGGAGGGGA |
| | | AACGGGTAGCAATGGTGGGAGATGGAATCAATGACTCCCCAGCTC |
| | | TGGCAATGGCTAATGTGGGAATTGCTATTGGCACAGGCACAGATG |
| | | TAGCCATTGAAGCAGCTGATGTGGTTTTGATAAGGAATGATCTTCT |
| | | GGATGTAGTGGCAAGTATTGACTTATCAAGAAAGACAGTCAAGAG |
| | | GATTCGGATAAATTTTGTCTTTGCTCTAATTTATAATCTGGTTGGAA |
| | | TTCCCATAGCTGCTGGAGTTTTTATGCCCATTGGTTTGGTTTTGCAG |
| | | CCCTGGATGGGATCTGCAGCAATGGCTGCTTCATCTGTTTCTGTAG |
| | | TACTTTCTTCTCTCCAGCTCAAGTGCTATAAGAAGCCTGACCTGGA |
| | | GAGGTATGAGGCACAGGCGCATGGCCACATGAAGCCCCTGACGGC |
| | | ATCCCAGGTCAGTGTGCACATAGGCATGGATGACAGGTGGCGGGA |
| | | CTCCCCCAGGGCCACACCATGGGACCAGGTCAGCTATGTCAGCCA |
| | | GGTGTCGCTGTCCTCCCTGACGTCCGACAAGCCATCTCGGCACAGC |
| | | GCTGCAGCAGACGATGATGGGGACAAGTGGTCTCTGCTCCTGAAT |
| | | GGCAGGGATGAGGAGCAGTACATCTGAGAATTCGATATCAAGCTT |
| | | ATCGATAATCAACCTCTGGATTACAAAATTTGTGAAAGATTGACTG |
| | | GTATTCTTAACTATGTTGCTCCTTTTACGCTATGTGGATACGCTGCT |
| | | TTAATGCCTTTGTATCATGCTATTGCTTCCCGTATGGCTTTCATTTT |
| | | CTCCTCCTTGTATAAATCCTGGTTGCTGTCTCTTTATGAGGAGTTGT |
| | | GGCCCGTTGTCAGGCAACGTGGCGTGGTGTGCACTGTGTTTGCTGA |
| | | CGCAACCCCCACTGGTTGGGGCACCTGCAGGAATAAAAGATCTTT |
| | | ATTTTCATTAGATCTGTGTGTTGGTTTTTTGTGTGTCTAGAGTAGAT |
| | | AAGTAGCATGGCGGGTTAATCATTAACTACAAGGAACCCCTAGTG |
| | | ATGGAGTTGGCCACTCCCTCTCTGCGCGCTCGCTCGCTCACTGAGG |
| | | CCGGGCGACCAAAGGTCGCCCGACGCCCGGGCTTTGCCCGGGCGG |
| | | CCTCAGTGAGCGAGCGAGCGCGCAGAGAGGGAGTGGCCAA |
| 97 | A44 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGATCCCATAAAACTCTAGCCTCCCAGAGGGA |
| | | CCCGAGTTGCTGGTGCTTCCCCAGGCTGGAGATTGAGTTAATATTA |
| | | ACAGGCCCAAGGCGATGTGGGCTTGTGCAATCATAGGCCCGGCCT |
| | | CGCCAGGTCACCTGAGGAGTTAATGAATACATATCTCCTGAGTTAA |
| | | TAATTACCAGCGCGGGCCAAATAAATAATCCGCGAGGGGCAGCTG |
| | | GACTTTGGACTCCGCGCGCTGGTAATTATTAACCTCGCGAATATTG |
| | | ATTCGAGGCCGCGATTGCCGCAATCGCGAGGGGCAGGTGACCTTT |
| | | GCCCAGCGCGCGTTCGCCCCGCCCCGGACGGTATCGATAAGCTTA |
| | | GGAGCTTGGGCTGCAGGTCGAGGGTTAATCATTAAGTCGTTAATTT |
| | | TTGTGGCCCTTGCGATGTTTGCTCTGGTTAATAATCTCAGGACAAA |
| | | CAGAGGTTAATAATTTTCCAGATCTCTCTGAGCAATAGTATAAAAG |
| | | GCCAGCAGCAGCCTGACCACATCTCATCCTTGAGCCAAGAGGTAA |
| | | GGGTTTAAGGGATGGTTGGTTGGTGGGGTATTAATGTTTAATTACC |
| | | TGGAGCACCTGCCTGAAATCACTTTTTTTCAGGTTGGGGATCCGCC |
| | | ACCATGCCGGAACAAGAACGCCAGATCACAGCAAGAGAAGGAGC |
| | | AAGTAGGAAGATCCTGAGCAAGCTCTCCCTGCCCACAAGGGCCTG |
| | | GGAGCCGGCCATGAAGAAGTCTTTTGCCTTCGATAATGTCGGTTAT |
| | | GAAGGCGGGCTCGATGGCCTGGGCCCCAGCAGCCAGCCCCAGAAG |
| | | TGTTTCCTGCAGATCAAGGGCATGACCTGTGCCAGCTGTGTTAGCA |
| | | ATATCGAAAGAAACCTCCAAAAAGAGGCTGGAGTTCTGAGTGTTC |
| | | TAGTGGCTTTAATGGCCGGGAAAGCCGAGATCAAGTATGATCCCG |
| | | AGGTGATTCAGCCATTGGAGATTGCCCAGTTCATCCAGGATCTGGG |
| | | CTTCGAGGCCGCCGTGATGGAGGATTACGCCGGCTCTGATGGCAA |
| | | CATCGAACTGACCATCACCGGCATGACCTGTGCTTCCTGCGTGCAT |
| | | AATATCGAGAGCAAACTGACGCGGACCAATGGAATCACCTATGCC |
| | | AGCGTCGCCCTGGCAACCTCTAAGGCTCTGGTGAAGTTTGACCCCG |
| | | AAATCATTGGACCTAGAGACATCATCAAAATTATTGAAGAGATTG |
| | | GTTTCCACGCCAGCTTAGCTCAGCGGAATCCCAACGCACACCATCT |
| | | TGACCATAAGATGGAAATCAAACAGTGGAAGAAGAGCTTCTTGTG |
| | | TAGCCTGGTCTTCGGGATACCCGTCATGGCACTGATGATTTACATG |
| | | CTCATTCCGTCCAACGAGCCCCACCAGTCCATGGTGCTGGACCACA |
| | | ACATCATCCCTGGGCTGTCTATCCTCAACCTGATCTTCTTTATTCTG |
| | | TGCACATTTGTCCAGCTCTTGGGAGGCTGGTATTTCTATGTACAGG |
| | | CCTACAAATCCCTGAGGCATCGAAGCGCCAACATGGACGTTTTAAT |
| | | TGTGCTGGCTACATCCATTGCCTACGTGTACTCCCTGGTGATCTTA |
| | | GTGGTTGCTGTGGCTGAAAAGGCTGAGAGAAGCCCTGTCACCTTCT |
| | | TCGACACCCCCCCCATGCTCTTCGTGTTTATTGCACTGGGTCGCTG |
| | | GCTGGAGCATTTAGCCAAGTCCAAGACATCCGAGGCTCTGGCCAA |
| | | GCTGATGTCTCTACAGGCCACGGAGGCCACCGTGGTGACTCTGGGT |
| | | GAAGACAATTTGATAATACGCGAGGAACAGGTGCCTATGGAGCTT |
| | | GTGCAGCGCGGAGATATCGTCAAGGTGGTGCCCGGCGGCAAGTTC |
| | | CCAGTGGATGGAAAAGTGTTAGAGGGCAATACCATGGCCGATGAA |
| | | TCCCTCATCACTGGCGAGGCAATGCCGGTGACCAAGAAACCTGGA |
| | | AGTACTGTCATTGCAGGCAGCATCAATGCCCATGGGAGCGTCCTG |
| | | ATCAAGGCCACCCACGTGGGCAATGATACAACTCTCGCCCAGATC |
| | | GTCAAGCTAGTGGAAGAGGCTCAGATGTCTAAAGCTCCAATCCAG |
| | | CAACTGGCTGACCGCTTCAGCGGCTACTTTGTCCCCTTTATCATCAT |
| | | CATGAGCACCCTCACCCTGGTGGTGTGGATAGTCATAGGCTTCATT |
| | | GACTTTGGCGTGGTGCAGAGATACTTCCCCAACCCTAACAAACATA |
| | | TCTCCCAAACCGAGGTCATTATCAGATTCGCCTTTCAGACATCTAT |
| | | CACTGTGCTGTGCATAGCCTGCCCTTGCTCTCTGGGCCTGGCCACC |
| | | CCCACCGCCGTGATGGTTGGAACCGGGGTCGCTGCACAAAATGGG |
| | | ATCCTGATAAAAGGGGGCAAACCCCTGGAAATGGCACATAAGATT |
| | | AAAACAGTTATGTTTGATAAGACAGGCACCATTACTCACGGTGTCC |
| | | CTCGAGTCATGAGAGTGCTGCTGCTGGGGGACGTGGCTACACTGC |
| | | CACTGCGCAAGGTGCTGGCTGTGGTGGGCACAGCGGAGGCCTCCT |
| | | CAGAACACCCATTAGGCGTAGCTGTCACAAAATACTGTAAGGAGG |
| | | AGCTGGGGACAGAGACCCTCGGCTATTGTACAGACTTCCAGGCCG |
| | | TGCCTGGCTGCGGCATCGGCTGTAAGGTCAGCAATGTGGAGGGCA |
| | | TCCTGGCACACAGCGAGAGACCCCTCTCTGCCCCAGCCTCCCACCT |
| | | GAATGAAGCTGGGTCTCTGCCTGCTGAGAAGGACGCCGTCCCTCA |
| | | GACCTTCTCCGTGTTGATTGGGAACAGAGAGTGGCTTAGAAGGAA |
| | | TGGCCTGACCATCAGCTCTGACGTCTCGGATGCCATGACAGATCAC |
| | | GAGATGAAAGGCCAGACAGCCATACTGGTTGCCATCGACGGCGTG |
| | | CTCTGTGGCATGATTGCCATCGCTGATGCTGTTAAACAGGAAGCTG |
| | | CGCTGGCAGTCCACACCCTCCAGTCCATGGGAGTGGACGTGGTCCT |
| | | GATTACTGGAGACAACAGGAAAACCGCTCGGGCCATCGCTACCCA |
| | | AGTGGGGATAAATAAGGTTTTTGCAGAAGTCTTACCCTCCCACAAA |
| | | GTAGCCAAAGTGCAGGAGCTCCAGAACAAGGGTAAAAAGGTGGC |
| | | AATGGTGGGAGATGGTGTGAATGACAGCCCAGCTCTCGCGCAGGC |
| | | TGACATGGGCGTAGCCATAGGTACTGGAACAGATGTCGCGATTGA |
| | | AGCCGCTGACGTAGTCCTCATCCGAAACGATTTGCTGGATGTGGTG |
| | | GCCAGCATCCATCTGTCCAAGCGTACCGTGCGCCGGATCCGCATCA |
| | | ACCTGGTCTTGGCCCTCATCTATAATCTCGTGGGCATCCCCATCGC |
| | | TGCTGGGGTCTTCATGCCCATCGGCATTGTGCTGCAGCCCTGGATG |
| | | GGAAGCGCTGCCATGGCTGCTTCTTCTGTCTCCGTTGTGCTGTCCA |
| | | GCCTACAGCTGAAGTGCTATAAAAAGCCAGACCTCGAGCGCTACG |
| | | AAGCCCAGGCGCATGGCCATATGAAGCCCCTCACGGCTTCCCAGG |
| | | TCTCGGTTCACATTGGGATGGATGACAGATGGCGGGACTCCCCAC |
| | | GCGCCACACCCTGGGACCAGGTATCCTATGTGAGCCAGGTGAGCC |
| | | TCAGCTCCCTGACATCAGATAAACCGTCTAGGCACTCAGCTGCCGC |
| | | AGATGATGACGGTGACAAATGGAGCCTCCTCCTGAACGGCCGCGA |
| | | CGAGGAGCAGTACATCTAAGAATTCGATATCAAGCTTATCGATAA |
| | | TCAACCTCTGGATTACAAAATTTGTGAAAGATTGACTGGTATTCTT |
| | | AACTATGTTGCTCCTTTTACGCTATGTGGATACGCTGCTTTAATGCC |
| | | TTTGTATCATGCTATTGCTTCCCGTATGGCTTTCATTTTCTCCTCCTT |
| | | GTATAAATCCTGGTTGCTGTCTCTTTATGAGGAGTTGTGGCCCGTT |
| | | GTCAGGCAACGTGGCGTGGTGTGCACTGTGTTTGCTGACGCAACCC |
| | | CCACTGGTTGGGGCACCTGCAGGAATAAAAGATCTTTATTTTCATT |
| | | AGATCTGTGTGTTGGTTTTTTGTGTGTCTAGAGTAGATAAGTAGCA |
| | | TGGCGGGTTAATCATTAACTACAAGGAACCCCTAGTGATGGAGTT |
| | | GGCCACTCCCTCTCTGCGCGCTCGCTCGCTCACTGAGGCCGGGCGA |
| | | CCAAAGGTCGCCCGACGCCCGGGCTTTGCCCGGGCGGCCTCAGTG |
| | | AGCGAGCGAGCGCGCAGAGAGGGAGTGGCCAA |
| 98 | A45 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGGTACCGAGTTGCTGGTGCTTCCCCAGGCT |
| | | GGAGATTGAGTTAATATTAACAGGCCCAAGGCGATGTGGGCTTGT |
| | | GCAATCATAGGCCCGGCCTCGCCAGGTCACCTGAGGAGTTAATGA |
| | | ATACATATCTCCTGAGTTAATAATTACCAGCGCGGGCCAAATAAAT |
| | | AATCCGCGAGGGGCAGCTGGACTTTGGACTCCGCGCGCTGGTAAT |
| | | TATTAACCTCGCGAATATTGATTCGAGGCCGCGATTGCCGCAATCG |
| | | CGAGGGGCAGGTGACCTTTGCCCAGCGCGCGTTCGCCCCGCCCCG |
| | | GACGGTATCGATAAGCTTAGGAGCTTGGGCTGCAGGTCGAGGGTT |
| | | AATCATTAAGTCGTTAATTTTTGTGGCCCTTGCGATGTTTGCTCTGG |
| | | TTAATAATCTCAGGACAAACAGAGGTTAATAATTTTCCAGATCTCT |
| | | CTGAGCAATAGTATAAAAGGCCAGCAGCAGCCTGACCACATCTCA |
| | | TCCTGATCCACAAGCTCGAGCCGAGTGGTCGTTGAGCCAAGAGGT |
| | | AAGGGTTTAAGGGATGGTTGGTTGGTGGGGTATTAATGTTTAATTA |
| | | CCTGGAGCACCTGCCTGAAATCACTTTTTTTCAGGTTGGGGATCCG |
| | | CCACCATGCCGGAACAAGAACGCCAGATCACAGCAAGAGAAGGA |
| | | GCAAGTAGGAAGATCCTGAGCAAGCTCTCCCTGCCCACAAGGGCC |
| | | TGGGAGCCGGCCATGAAGAAGTCTTTTGCCTTCGATAATGTCGGTT |
| | | ATGAAGGCGGGCTCGATGGCCTGGGCCCCAGCAGCCAGCCCCAGA |
| | | AGTGTTTCCTGCAGATCAAGGGCATGACCTGTGCCAGCTGTGTTAG |
| | | CAATATCGAAAGAAACCTCCAAAAAGAGGCTGGAGTTCTGAGTGT |
| | | TCTAGTGGCTTTAATGGCCGGGAAAGCCGAGATCAAGTATGATCC |
| | | CGAGGTGATTCAGCCATTGGAGATTGCCCAGTTCATCCAGGATCTG |
| | | GGCTTCGAGGCCGCCGTGATGGAGGATTACGCCGGCTCTGATGGC |
| | | AACATCGAACTGACCATCACCGGCATGACCTGTGCTTCCTGCGTGC |
| | | ATAATATCGAGAGCAAACTGACGCGGACCAATGGAATCACCTATG |
| | | CCAGCGTCGCCCTGGCAACCTCTAAGGCTCTGGTGAAGTTTGACCC |
| | | CGAAATCATTGGACCTAGAGACATCATCAAAATTATTGAAGAGAT |
| | | TGGTTTCCACGCCAGCTTAGCTCAGCGGAATCCCAACGCACACCAT |
| | | CTTGACCATAAGATGGAAATCAAACAGTGGAAGAAGAGCTTCTTG |
| | | TGTAGCCTGGTCTTCGGGATACCCGTCATGGCACTGATGATTTACA |
| | | TGCTCATTCCGTCCAACGAGCCCCACCAGTCCATGGTGCTGGACCA |
| | | CAACATCATCCCTGGGCTGTCTATCCTCAACCTGATCTTCTTTATTC |
| | | TGTGCACATTTGTCCAGCTCTTGGGAGGCTGGTATTTCTATGTACA |
| | | GGCCTACAAATCCCTGAGGCATCGAAGCGCCAACATGGACGTTTT |
| | | AATTGTGCTGGCTACATCCATTGCCTACGTGTACTCCCTGGTGATC |
| | | TTAGTGGTTGCTGTGGCTGAAAAGGCTGAGAGAAGCCCTGTCACCT |
| | | TCTTCGACACCCCCCCCATGCTCTTCGTGTTTATTGCACTGGGTCGC |
| | | TGGCTGGAGCATTTAGCCAAGTCCAAGACATCCGAGGCTCTGGCC |
| | | AAGCTGATGTCTCTACAGGCCACGGAGGCCACCGTGGTGACTCTG |
| | | GGTGAAGACAATTTGATAATACGCGAGGAACAGGTGCCTATGGAG |
| | | CTTGTGCAGCGCGGAGATATCGTCAAGGTGGTGCCCGGCGGCAAG |
| | | TTCCCAGTGGATGGAAAAGTGTTAGAGGGCAATACCATGGCCGAT |
| | | GAATCCCTCATCACTGGCGAGGCAATGCCGGTGACCAAGAAACCT |
| | | GGAAGTACTGTCATTGCAGGCAGCATCAATGCCCATGGGAGCGTC |
| | | CTGATCAAGGCCACCCACGTGGGCAATGATACAACTCTCGCCCAG |
| | | ATCGTCAAGCTAGTGGAAGAGGCTCAGATGTCTAAAGCTCCAATC |
| | | CAGCAACTGGCTGACCGCTTCAGCGGCTACTTTGTCCCCTTTATCA |
| | | TCATCATGAGCACCCTCACCCTGGTGGTGTGGATAGTCATAGGCTT |
| | | CATTGACTTTGGCGTGGTGCAGAGATACTTCCCCAACCCTAACAAA |
| | | CATATCTCCCAAACCGAGGTCATTATCAGATTCGCCTTTCAGACAT |
| | | CTATCACTGTGCTGTGCATAGCCTGCCCTTGCTCTCTGGGCCTGGC |
| | | CACCCCCACCGCCGTGATGGTTGGAACCGGGGTCGCTGCACAAAA |
| | | TGGGATCCTGATAAAAGGGGGCAAACCCCTGGAAATGGCACATAA |
| | | GATTAAAACAGTTATGTTTGATAAGACAGGCACCATTACTCACGGT |
| | | GTCCCTCGAGTCATGAGAGTGCTGCTGCTGGGGGACGTGGCTACA |
| | | CTGCCACTGCGCAAGGTGCTGGCTGTGGTGGGCACAGCGGAGGCC |
| | | TCCTCAGAACACCCATTAGGCGTAGCTGTCACAAAATACTGTAAG |
| | | GAGGAGCTGGGGACAGAGACCCTCGGCTATTGTACAGACTTCCAG |
| | | GCCGTGCCTGGCTGCGGCATCGGCTGTAAGGTCAGCAATGTGGAG |
| | | GGCATCCTGGCACACAGCGAGAGACCCCTCTCTGCCCCAGCCTCCC |
| | | ACCTGAATGAAGCTGGGTCTCTGCCTGCTGAGAAGGACGCCGTCC |
| | | CTCAGACCTTCTCCGTGTTGATTGGGAACAGAGAGTGGCTTAGAAG |
| | | GAATGGCCTGACCATCAGCTCTGACGTCTCGGATGCCATGACAGAT |
| | | CACGAGATGAAAGGCCAGACAGCCATACTGGTTGCCATCGACGGC |
| | | GTGCTCTGTGGCATGATTGCCATCGCTGATGCTGTTAAACAGGAAG |
| | | CTGCGCTGGCAGTCCACACCCTCCAGTCCATGGGAGTGGACGTGGT |
| | | CCTGATTACTGGAGACAACAGGAAAACCGCTCGGGCCATCGCTAC |
| | | CCAAGTGGGGATAAATAAGGTTTTTGCAGAAGTCTTACCCTCCCAC |
| | | AAAGTAGCCAAAGTGCAGGAGCTCCAGAACAAGGGTAAAAAGGT |
| | | GGCAATGGTGGGAGATGGTGTGAATGACAGCCCAGCTCTCGCGCA |
| | | GGCTGACATGGGCGTAGCCATAGGTACTGGAACAGATGTCGCGAT |
| | | TGAAGCCGCTGACGTAGTCCTCATCCGAAACGATTTGCTGGATGTG |
| | | GTGGCCAGCATCCATCTGTCCAAGCGTACCGTGCGCCGGATCCGCA |
| | | TCAACCTGGTCTTGGCCCTCATCTATAATCTCGTGGGCATCCCCAT |
| | | CGCTGCTGGGGTCTTCATGCCCATCGGCATTGTGCTGCAGCCCTGG |
| | | ATGGGAAGCGCTGCCATGGCTGCTTCTTCTGTCTCCGTTGTGCTGT |
| | | CCAGCCTACAGCTGAAGTGCTATAAAAAGCCAGACCTCGAGCGCT |
| | | ACGAAGCCCAGGCGCATGGCCATATGAAGCCCCTCACGGCTTCCC |
| | | AGGTCTCGGTTCACATTGGGATGGATGACAGATGGCGGGACTCCC |
| | | CACGCGCCACACCCTGGGACCAGGTATCCTATGTGAGCCAGGTGA |
| | | GCCTCAGCTCCCTGACATCAGATAAACCGTCTAGGCACTCAGCTGC |
| | | CGCAGATGATGACGGTGACAAATGGAGCCTCCTCCTGAACGGCCG |
| | | CGACGAGGAGCAGTACATCTAAGAATTCGATATCAAGCTTATCGA |
| | | TAATCAACCTCTGGATTACAAAATTTGTGAAAGATTGACTGGTATT |
| | | CTTAACTATGTTGCTCCTTTTACGCTATGTGGATACGCTGCTTTAAT |
| | | GCCTTTGTATCATGCTATTGCTTCCCGTATGGCTTTCATTTTCTCCT |
| | | CCTTGTATAAATCCTGGTTGCTGTCTCTTTATGAGGAGTTGTGGCCC |
| | | GTTGTCAGGCAACGTGGCGTGGTGTGCACTGTGTTTGCTGACGCAA |
| | | CCCCCACTGGTTGGGGCACCTGCAGGAATAAAAGATCTTTATTTTC |
| | | ATTAGATCTGTGTGTTGGTTTTTTGTGTGTCTAGAGTAGATAAGTA |
| | | GCATGGCGGGTTAATCATTAACTACAAGGAACCCCTAGTGATGGA |
| | | GTTGGCCACTCCCTCTCTGCGCGCTCGCTCGCTCACTGAGGCCGGG |
| | | CGACCAAAGGTCGCCCGACGCCCGGGCTTTGCCCGGGCGGCCTCA |
| | | GTGAGCGAGCGAGCGCGCAGAGAGGGAGTGGCCAA |
| 99 | A46 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGGTACCGAGTTGCTGGTGCTTCCCCAGGCT |
| | | GGAGATTGAGTTAATATTAACAGGCCCAAGGCGATGTGGGCTTGT |
| | | GCAATCATAGGCCCGGCCTCGCCAGGTCACCTGAGGAGTTAATGA |
| | | ATACATATCTCCTGAGTTAATAATTACCAGCGCGGGCCAAATAAAT |
| | | AATCCGCGAGGGGCAGCTGGACTTTGGACTCCGCGCGCTGGTAAT |
| | | TATTAACCTCGCGAATATTGATTCGAGGCCGCGATTGCCGCAATCG |
| | | CGAGGGGCAGGTGACCTTTGCCCAGCGCGCGTTCGCCCCGCCCCG |
| | | GACGGTATCGATAAGCTTAGGAGCTTGGGCTGCAGGTCGAGGGTT |
| | | AATCATTAAGTCGTTAATTTTTGTGGCCCTTGCGATGTTTGCTCTGG |
| | | TTAATAATCTCAGGACAAACAGAGGTTAATAATTTTCCAGATCTCT |
| | | CTGAGCAATAGTATAAAAGGCCAGCAGCAGCCTGACCACATCTCA |
| | | TCCTGATCCACAAGCTCGAGCCGAGTGGTCGTTGAGCCAAGAGGT |
| | | AAGGGTTTAAGGGATGGTTGGTTGGTGGGGTATTAATGTTTAATTA |
| | | CCTGGAGCACCTGCCTGAAATCACTTTTTTTCAGGTTGGGGATCCG |
| | | CCACCATGCCCGAACAGGAAAGACAGATTACCGCCCGCGAGGGCG |
| | | CTTCCCGCAAAATTCTGAGCAAACTGAGCCTGCCCACCCGCGCCTG |
| | | GGAACCTGCCATGAAGAAAAGCTTTGCTTTTGATAATGTCGGCTAT |
| | | GAGGGCGGCCTGGACGGCCTGGGCCCTAGCAGCCAGGTGGCTACC |
| | | ACCCTGATTGCCATTGCCGGCATGACCTGCGCCAGCTGCGTGCATA |
| | | GCATTGAGGGCATGATTAGCCAGCTGGAGGGCGTGCAGCAGATTT |
| | | CCGTGAGCCTGGCTGAGGGCACCGCTACCGTGCTGTATAATCCCTC |
| | | CGTGATTAGCCCCGAAGAACTGCGCGCTGCCATTGAAGATATGGG |
| | | CTTTGAAGCCTCCGTGGTGTCCGAAAGCTGCAGCACCAATCCCCTG |
| | | GGCAATCATTCCGCCGGCAATAGCATGGTGCAGACCACCGACGGC |
| | | ACCCCCACCTCCGTGCAGGAAGTGGCTCCCCATACCGGCCGCCTGC |
| | | CCGCCAATCATGCCCCTGATATTCTGGCTAAAAGCCCCCAGAGCAC |
| | | CCGCGCCGTGGCTCCTCAGAAATGCTTTCTGCAGATTAAGGGCATG |
| | | ACCTGCGCTAGCTGCGTGAGCAATATTGAACGCAATCTGCAGAAA |
| | | GAAGCCGGCGTGCTGTCCGTGCTGGTGGCCCTGATGGCCGGCAAA |
| | | GCTGAAATCAAATATGATCCCGAAGTGATTCAGCCCCTGGAAATT |
| | | GCCCAGTTCATTCAGGATCTGGGCTTTGAGGCTGCCGTGATGGAAG |
| | | ATTATGCCGGCTCCGACGGCAATATTGAACTGACCATTACCGGCAT |
| | | GACCTGCGCCAGCTGCGTGCATAATATTGAAAGCAAACTGACCCG |
| | | CACCAACGGCATTACCTATGCTTCCGTGGCCCTGGCTACCAGCAAA |
| | | GCCCTGGTGAAATTTGATCCTGAAATTATCGGCCCCCGCGATATTA |
| | | TCAAAATTATTGAAGAAATCGGCTTTCACGCCAGCCTGGCTCAGCG |
| | | CAATCCCAATGCCCATCATCTGGATCATAAGATGGAAATCAAACA |
| | | GTGGAAGAAAAGCTTTCTGTGCAGCCTGGTGTTCGGCATTCCTGTG |
| | | ATGGCCCTGATGATCTATATGCTGATTCCCAGCAATGAACCCCATC |
| | | AGAGCATGGTGCTGGATCATAATATTATTCCCGGCCTGAGCATTCT |
| | | GAATCTGATTTTCTTTATCCTGTGCACCTTTGTGCAGCTGCTGGGCG |
| | | GCTGGTATTTCTATGTGCAGGCTTATAAAAGCCTGCGCCACCGCTC |
| | | CGCCAATATGGATGTGCTGATTGTGCTGGCCACCAGCATTGCTTAT |
| | | GTCTATAGCCTGGTCATTCTGGTGGTGGCCGTGGCCGAGAAAGCTG |
| | | AACGCAGCCCCGTGACCTTCTTTGATACCCCTCCCATGCTGTTCGT |
| | | GTTTATCGCTCTGGGCCGCTGGCTGGAACATCTGGCCAAAAGCAA |
| | | AACCTCCGAAGCCCTGGCTAAACTGATGAGCCTGCAGGCCACCGA |
| | | AGCCACCGTGGTGACCCTGGGCGAAGATAATCTGATTATCCGCGA |
| | | AGAACAGGTGCCTATGGAACTGGTGCAGCGCGGCGATATTGTGAA |
| | | AGTGGTGCCCGGCGGCAAATTTCCCGTGGACGGCAAAGTGCTGGA |
| | | GGGCAATACCATGGCTGATGAATCCCTGATTACCGGCGAAGCTAT |
| | | GCCTGTGACCAAGAAACCCGGCAGCACCGTGATTGCCGGCAGCAT |
| | | TAATGCTCACGGCTCCGTGCTGATCAAGGCTACCCATGTCGGCAAT |
| | | GATACCACCCTGGCCCAGATTGTGAAACTGGTGGAAGAAGCCCAG |
| | | ATGAGCAAAGCCCCTATTCAGCAGCTGGCTGACCGCTTTTCCGGCT |
| | | ATTTTGTGCCCTTTATCATTATTATGAGCACCCTGACCCTGGTGGTG |
| | | TGGATTGTGATCGGCTTCATTGATTTCGGCGTGGTGCAGAGATATT |
| | | TTCCTAATCCCAACAAACATATTAGCCAGACCGAAGTGATTATCCG |
| | | CTTTGCTTTTCAGACCAGCATTACCGTGCTGTGCATTGCTTGCCCCT |
| | | GCAGCCTGGGCCTGGCTACCCCCACCGCCGTCATGGTCGGCACCG |
| | | GCGTGGCTGCCCAGAACGGCATTCTGATTAAGGGCGGCAAACCTC |
| | | TGGAAATGGCCCACAAAATCAAAACCGTGATGTTTGATAAAACCG |
| | | GCACCATTACCCACGGCGTGCCCCGCGTGATGCGCGTGCTGCTGCT |
| | | GGGCGATGTGGCTACCCTGCCCCTGCGCAAAGTGCTGGCCGTGGTC |
| | | GGCACCGCCGAAGCTAGCTCCGAACATCCCCTGGGCGTGGCCGTG |
| | | ACCAAATATTGCAAAGAAGAACTGGGCACCGAAACCCTGGGCTAT |
| | | TGCACCGATTTTCAGGCCGTCCCTGGCTGCGGCATCGGCTGCAAAG |
| | | TGAGCAATGTGGAGGGCATTCTGGCCCATTCCGAAAGACCCCTGTC |
| | | CGCCCCTGCTAGCCATCTGAATGAAGCCGGCAGCCTGCCTGCTGAG |
| | | AAAGATGCCGTGCCCCAGACCTTTTCCGTGCTGATCGGCAACCGCG |
| | | AATGGCTGCGCCGCAACGGCCTGACCATTAGCTCCGATGTCTCCGA |
| | | TGCCATGACCGATCATGAAATGAAAGGCCAGACCGCCATTCTGGT |
| | | GGCCATTGACGGCGTGCTGTGCGGCATGATTGCCATTGCCGATGCC |
| | | GTGAAACAGGAGGCTGCCCTGGCTGTGCATACCCTGCAGAGCATG |
| | | GGCGTGGATGTGGTGCTGATTACCGGCGATAACCGCAAAACCGCT |
| | | CGCGCCATTGCCACCCAGGTCGGCATTAACAAAGTGTTTGCTGAAG |
| | | TGCTGCCCAGCCATAAAGTGGCTAAAGTGCAGGAACTGCAGAACA |
| | | AAGGCAAGAAAGTGGCTATGGTCGGCGACGGCGTGAATGATAGCC |
| | | CTGCCCTGGCCCAGGCCGATATGGGCGTGGCTATCGGCACCGGCA |
| | | CCGATGTGGCTATTGAGGCTGCCGATGTGGTGCTGATCCGCAATGA |
| | | TCTGCTGGATGTGGTGGCTAGCATTCATCTGAGCAAACGCACCGTC |
| | | CGCCGCATCCGCATTAATCTGGTGCTGGCCCTGATCTATAATCTGG |
| | | TCGGCATTCCCATCGCCGCCGGCGTGTTTATGCCCATCGGCATTGT |
| | | GCTGCAGCCCTGGATGGGCAGCGCCGCTATGGCCGCTAGCTCCGT |
| | | GTCCGTGGTGCTGAGCAGCCTGCAGCTGAAATGCTATAAGAAACC |
| | | CGATCTGGAAAGATATGAAGCTCAGGCTCACGGCCATATGAAACC |
| | | CCTGACCGCCAGCCAGGTGTCCGTGCATATCGGCATGGATGACCG |
| | | CTGGCGCGATAGCCCCCGCGCCACCCCTTGGGATCAGGTGAGCTAT |
| | | GTGAGCCAGGTGTCCCTGAGCTCCCTGACCTCCGATAAACCCTCCC |
| | | GCCATTCCGCCGCTGCCGATGATGACGGCGATAAGTGGAGCCTGC |
| | | TGCTGAACGGCCGCGATGAAGAACAGTATATCTAAGTCCATAAAG |
| | | TAGGAAACACTACATCATCCATAAAGTAGGAAACACTACATCATC |
| | | CATAAAGTAGGAAACACTACATCATCCATAAAGTAGGAAACACTA |
| | | CAGAATTCGATATCAAGCTTATCGATAATCAACCTCTGGATTACAA |
| | | AATTTGTGAAAGATTGACTGGTATTCTTAACTATGTTGCTCCTTTTA |
| | | CGCTATGTGGATACGCTGCTTTAATGCCTTTGTATCATGCTATTGCT |
| | | TCCCGTATGGCTTTCATTTTCTCCTCCTTGTATAAATCCTGGTTGCT |
| | | GTCTCTTTATGAGGAGTTGTGGCCCGTTGTCAGGCAACGTGGCGTG |
| | | GTGTGCACTGTGTTTGCTGACGCAACCCCCACTGGTTGGGGCACCT |
| | | GCAGGAATAAAAGATCTTTATTTTCATTAGATCTGTGTGTTGGTTTT |
| | | TTGTGTGTCTAGAGTAGATAAGTAGCATGGCGGGTTAATCATTAAC |
| | | TACAAGGAACCCCTAGTGATGGAGTTGGCCACTCCCTCTCTGCGCG |
| | | CTCGCTCGCTCACTGAGGCCGGGCGACCAAAGGTCGCCCGACGCC |
| | | CGGGCTTTGCCCGGGCGGCCTCAGTGAGCGAGCGAGCGCGCAGAG |
| | | AGGGAGTGGCCAA |
| 100 | A47 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGGTACCGTTAATCATTAAGTCGTTAATTTT |
| | | TGTGGCCCTTGCGATGTTTGCTCTGGTTAATAATCTCAGGACAAAC |
| | | AGAGGTTAATAATTTTCCAGATCTCTCTGAGCAATAGTATAAAAGG |
| | | CCAGCAGCAGCCTGACCACATCTCATCCTTGATCCACAAGCTCGAG |
| | | CCGAGTGGTCGTGAGCCAAGAGGTAAGGGTTTAAGGGATGGTTGG |
| | | TTGGTGGGGTATTAATGTTTAATTACCTGGAGCACCTGCCTGAAAT |
| | | CACTTTTTTTCAGGTTGGGGATCCGCCACCATGCCGGAACAAGAAC |
| | | GCCAGATCACAGCAAGAGAAGGAGCAAGTAGGAAGATCCTGAGC |
| | | AAGCTCTCCCTGCCCACAAGGGCCTGGGAGCCGGCCATGAAGAAG |
| | | TCTTTTGCCTTCGATAATGTCGGTTATGAAGGCGGGCTCGATGGCC |
| | | TGGGCCCCAGCAGCCAGCCCCAGAAGTGTTTCCTGCAGATCAAGG |
| | | GCATGACCTGTGCCAGCTGTGTTAGCAATATCGAAAGAAACCTCC |
| | | AAAAAGAGGCTGGAGTTCTGAGTGTTCTAGTGGCTTTAATGGCCG |
| | | GGAAAGCCGAGATCAAGTATGATCCCGAGGTGATTCAGCCATTGG |
| | | AGATTGCCCAGTTCATCCAGGATCTGGGCTTCGAGGCCGCCGTGAT |
| | | GGAGGATTACGCCGGCTCTGATGGCAACATCGAACTGACCATCAC |
| | | CGGCATGACCTGTGCTTCCTGCGTGCATAATATCGAGAGCAAACTG |
| | | ACGCGGACCAATGGAATCACCTATGCCAGCGTCGCCCTGGCAACC |
| | | TCTAAGGCTCTGGTGAAGTTTGACCCCGAAATCATTGGACCTAGAG |
| | | ACATCATCAAAATTATTGAAGAGATTGGTTTCCACGCCAGCTTAGC |
| | | TCAGCGGAATCCCAACGCACACCATCTTGACCATAAGATGGAAAT |
| | | CAAACAGTGGAAGAAGAGCTTCTTGTGTAGCCTGGTCTTCGGGAT |
| | | ACCCGTCATGGCACTGATGATTTACATGCTCATTCCGTCCAACGAG |
| | | CCCCACCAGTCCATGGTGCTGGACCACAACATCATCCCTGGGCTGT |
| | | CTATCCTCAACCTGATCTTCTTTATTCTGTGCACATTTGTCCAGCTC |
| | | TTGGGAGGCTGGTATTTCTATGTACAGGCCTACAAATCCCTGAGGC |
| | | ATCGAAGCGCCAACATGGACGTTTTAATTGTGCTGGCTACATCCAT |
| | | TGCCTACGTGTACTCCCTGGTGATCTTAGTGGTTGCTGTGGCTGAA |
| | | AAGGCTGAGAGAAGCCCTGTCACCTTCTTCGACACCCCCCCCATGC |
| | | TCTTCGTGTTTATTGCACTGGGTCGCTGGCTGGAGCATTTAGCCAA |
| | | GTCCAAGACATCCGAGGCTCTGGCCAAGCTGATGTCTCTACAGGCC |
| | | ACGGAGGCCACCGTGGTGACTCTGGGTGAAGACAATTTGATAATA |
| | | CGCGAGGAACAGGTGCCTATGGAGCTTGTGCAGCGCGGAGATATC |
| | | GTCAAGGTGGTGCCCGGCGGCAAGTTCCCAGTGGATGGAAAAGTG |
| | | TTAGAGGGCAATACCATGGCCGATGAATCCCTCATCACTGGCGAG |
| | | GCAATGCCGGTGACCAAGAAACCTGGAAGTACTGTCATTGCAGGC |
| | | AGCATCAATGCCCATGGGAGCGTCCTGATCAAGGCCACCCACGTG |
| | | GGCAATGATACAACTCTCGCCCAGATCGTCAAGCTAGTGGAAGAG |
| | | GCTCAGATGTCTAAAGCTCCAATCCAGCAACTGGCTGACCGCTTCA |
| | | GCGGCTACTTTGTCCCCTTTATCATCATCATGAGCACCCTCACCCTG |
| | | GTGGTGTGGATAGTCATAGGCTTCATTGACTTTGGCGTGGTGCAGA |
| | | GATACTTCCCCAACCCTAACAAACATATCTCCCAAACCGAGGTCAT |
| | | TATCAGATTCGCCTTTCAGACATCTATCACTGTGCTGTGCATAGCC |
| | | TGCCCTTGCTCTCTGGGCCTGGCCACCCCCACCGCCGTGATGGTTG |
| | | GAACCGGGGTCGCTGCACAAAATGGGATCCTGATAAAAGGGGGCA |
| | | AACCCCTGGAAATGGCACATAAGATTAAAACAGTTATGTTTGATA |
| | | AGACAGGCACCATTACTCACGGTGTCCCTCGAGTCATGAGAGTGCT |
| | | GCTGCTGGGGGACGTGGCTACACTGCCACTGCGCAAGGTGCTGGC |
| | | TGTGGTGGGCACAGCGGAGGCCTCCTCAGAACACCCATTAGGCGT |
| | | AGCTGTCACAAAATACTGTAAGGAGGAGCTGGGGACAGAGACCCT |
| | | CGGCTATTGTACAGACTTCCAGGCCGTGCCTGGCTGCGGCATCGGC |
| | | TGTAAGGTCAGCAATGTGGAGGGCATCCTGGCACACAGCGAGAGA |
| | | CCCCTCTCTGCCCCAGCCTCCCACCTGAATGAAGCTGGGTCTCTGC |
| | | CTGCTGAGAAGGACGCCGTCCCTCAGACCTTCTCCGTGTTGATTGG |
| | | GAACAGAGAGTGGCTTAGAAGGAATGGCCTGACCATCAGCTCTGA |
| | | CGTCTCGGATGCCATGACAGATCACGAGATGAAAGGCCAGACAGC |
| | | CATACTGGTTGCCATCGACGGCGTGCTCTGTGGCATGATTGCCATC |
| | | GCTGATGCTGTTAAACAGGAAGCTGCGCTGGCAGTCCACACCCTCC |
| | | AGTCCATGGGAGTGGACGTGGTCCTGATTACTGGAGACAACAGGA |
| | | AAACCGCTCGGGCCATCGCTACCCAAGTGGGGATAAATAAGGTTT |
| | | TTGCAGAAGTCTTACCCTCCCACAAAGTAGCCAAAGTGCAGGAGC |
| | | TCCAGAACAAGGGTAAAAAGGTGGCAATGGTGGGAGATGGTGTGA |
| | | ATGACAGCCCAGCTCTCGCGCAGGCTGACATGGGCGTAGCCATAG |
| | | GTACTGGAACAGATGTCGCGATTGAAGCCGCTGACGTAGTCCTCAT |
| | | CCGAAACGATTTGCTGGATGTGGTGGCCAGCATCCATCTGTCCAAG |
| | | CGTACCGTGCGCCGGATCCGCATCAACCTGGTCTTGGCCCTCATCT |
| | | ATAATCTCGTGGGCATCCCCATCGCTGCTGGGGTCTTCATGCCCAT |
| | | CGGCATTGTGCTGCAGCCCTGGATGGGAAGCGCTGCCATGGCTGCT |
| | | TCTTCTGTCTCCGTTGTGCTGTCCAGCCTACAGCTGAAGTGCTATA |
| | | AAAAGCCAGACCTCGAGCGCTACGAAGCCCAGGCGCATGGCCATA |
| | | TGAAGCCCCTCACGGCTTCCCAGGTCTCGGTTCACATTGGGATGGA |
| | | TGACAGATGGCGGGACTCCCCACGCGCCACACCCTGGGACCAGGT |
| | | ATCCTATGTGAGCCAGGTGAGCCTCAGCTCCCTGACATCAGATAAA |
| | | CCGTCTAGGCACTCAGCTGCCGCAGATGATGACGGTGACAAATGG |
| | | AGCCTCCTCCTGAACGGCCGCGACGAGGAGCAGTACATCTAAGAA |
| | | TTCGATATCAAGCTTATCGATAATCAACCTCTGGATTACAAAATTT |
| | | GTGAAAGATTGACTGGTATTCTTAACTATGTTGCTCCTTTTACGCTA |
| | | TGTGGATACGCTGCTTTAATGCCTTTGTATCATGCTATTGCTTCCCG |
| | | TATGGCTTTCATTTTCTCCTCCTTGTATAAATCCTGGTTGCTGTCTC |
| | | TTTATGAGGAGTTGTGGCCCGTTGTCAGGCAACGTGGCGTGGTGTG |
| | | CACTGTGTTTGCTGACGCAACCCCCACTGGTTGGGGCACCTGCAGG |
| | | AATAAAAGATCTTTATTTTCATTAGATCTGTGTGTTGGTTTTTTGTG |
| | | TGTCTAGAGTAGATAAGTAGCATGGCGGGTTAATCATTAACTACA |
| | | AGGAACCCCTAGTGATGGAGTTGGCCACTCCCTCTCTGCGCGCTCG |
| | | CTCGCTCACTGAGGCCGGGCGACCAAAGGTCGCCCGACGCCCGGG |
| | | CTTTGCCCGGGCGGCCTCAGTGAGCGAGCGAGCGCGCAGAGAGGG |
| | | AGTGGCCAA |
| 101 | A48 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGGTACCGTTAATCATTAAGTCGTTAATTTT |
| | | TGTGGCCCTTGCGATGTTTGCTCTGGTTAATAATCTCAGGACAAAC |
| | | AGAGGTTAATAATTTTCCAGATCTCTCTGAGCAATAGTATAAAAGG |
| | | CCAGCAGCAGCCTGACCACATCTCATCCTTGATCCACAAGCTCGAG |
| | | CCGAGTGGTCGTGAGCCAAGAGGTAAGGGTTTAAGGGATGGTTGG |
| | | TTGGTGGGGTATTAATGTTTAATTACCTGGAGCACCTGCCTGAAAT |
| | | CACTTTTTTTCAGGTTGGGGATCCGCCACCATGCCCGAACAGGAAA |
| | | GACAGATTACCGCCCGCGAGGGCGCTTCCCGCAAAATTCTGAGCA |
| | | AACTGAGCCTGCCCACCCGCGCCTGGGAACCTGCCATGAAGAAAA |
| | | GCTTTGCTTTTGATAATGTCGGCTATGAGGGCGGCCTGGACGGCCT |
| | | GGGCCCTAGCAGCCAGGTGGCTACCACCCTGATTGCCATTGCCGGC |
| | | ATGACCTGCGCCAGCTGCGTGCATAGCATTGAGGGCATGATTAGC |
| | | CAGCTGGAGGGCGTGCAGCAGATTTCCGTGAGCCTGGCTGAGGGC |
| | | ACCGCTACCGTGCTGTATAATCCCTCCGTGATTAGCCCCGAAGAAC |
| | | TGCGCGCTGCCATTGAAGATATGGGCTTTGAAGCCTCCGTGGTGTC |
| | | CGAAAGCTGCAGCACCAATCCCCTGGGCAATCATTCCGCCGGCAA |
| | | TAGCATGGTGCAGACCACCGACGGCACCCCCACCTCCGTGCAGGA |
| | | AGTGGCTCCCCATACCGGCCGCCTGCCCGCCAATCATGCCCCTGAT |
| | | ATTCTGGCTAAAAGCCCCCAGAGCACCCGCGCCGTGGCTCCTCAG |
| | | AAATGCTTTCTGCAGATTAAGGGCATGACCTGCGCTAGCTGCGTGA |
| | | GCAATATTGAACGCAATCTGCAGAAAGAAGCCGGCGTGCTGTCCG |
| | | TGCTGGTGGCCCTGATGGCCGGCAAAGCTGAAATCAAATATGATC |
| | | CCGAAGTGATTCAGCCCCTGGAAATTGCCCAGTTCATTCAGGATCT |
| | | GGGCTTTGAGGCTGCCGTGATGGAAGATTATGCCGGCTCCGACGG |
| | | CAATATTGAACTGACCATTACCGGCATGACCTGCGCCAGCTGCGTG |
| | | CATAATATTGAAAGCAAACTGACCCGCACCAACGGCATTACCTAT |
| | | GCTTCCGTGGCCCTGGCTACCAGCAAAGCCCTGGTGAAATTTGATC |
| | | CTGAAATTATCGGCCCCCGCGATATTATCAAAATTATTGAAGAAAT |
| | | CGGCTTTCACGCCAGCCTGGCTCAGCGCAATCCCAATGCCCATCAT |
| | | CTGGATCATAAGATGGAAATCAAACAGTGGAAGAAAAGCTTTCTG |
| | | TGCAGCCTGGTGTTCGGCATTCCTGTGATGGCCCTGATGATCTATA |
| | | TGCTGATTCCCAGCAATGAACCCCATCAGAGCATGGTGCTGGATCA |
| | | TAATATTATTCCCGGCCTGAGCATTCTGAATCTGATTTTCTTTATCC |
| | | TGTGCACCTTTGTGCAGCTGCTGGGCGGCTGGTATTTCTATGTGCA |
| | | GGCTTATAAAAGCCTGCGCCACCGCTCCGCCAATATGGATGTGCTG |
| | | ATTGTGCTGGCCACCAGCATTGCTTATGTCTATAGCCTGGTCATTCT |
| | | GGTGGTGGCCGTGGCCGAGAAAGCTGAACGCAGCCCCGTGACCTT |
| | | CTTTGATACCCCTCCCATGCTGTTCGTGTTTATCGCTCTGGGCCGCT |
| | | GGCTGGAACATCTGGCCAAAAGCAAAACCTCCGAAGCCCTGGCTA |
| | | AACTGATGAGCCTGCAGGCCACCGAAGCCACCGTGGTGACCCTGG |
| | | GCGAAGATAATCTGATTATCCGCGAAGAACAGGTGCCTATGGAAC |
| | | TGGTGCAGCGCGGCGATATTGTGAAAGTGGTGCCCGGCGGCAAAT |
| | | TTCCCGTGGACGGCAAAGTGCTGGAGGGCAATACCATGGCTGATG |
| | | AATCCCTGATTACCGGCGAAGCTATGCCTGTGACCAAGAAACCCG |
| | | GCAGCACCGTGATTGCCGGCAGCATTAATGCTCACGGCTCCGTGCT |
| | | GATCAAGGCTACCCATGTCGGCAATGATACCACCCTGGCCCAGATT |
| | | GTGAAACTGGTGGAAGAAGCCCAGATGAGCAAAGCCCCTATTCAG |
| | | CAGCTGGCTGACCGCTTTTCCGGCTATTTTGTGCCCTTTATCATTAT |
| | | TATGAGCACCCTGACCCTGGTGGTGTGGATTGTGATCGGCTTCATT |
| | | GATTTCGGCGTGGTGCAGAGATATTTTCCTAATCCCAACAAACATA |
| | | TTAGCCAGACCGAAGTGATTATCCGCTTTGCTTTTCAGACCAGCAT |
| | | TACCGTGCTGTGCATTGCTTGCCCCTGCAGCCTGGGCCTGGCTACC |
| | | CCCACCGCCGTCATGGTCGGCACCGGCGTGGCTGCCCAGAACGGC |
| | | ATTCTGATTAAGGGCGGCAAACCTCTGGAAATGGCCCACAAAATC |
| | | AAAACCGTGATGTTTGATAAAACCGGCACCATTACCCACGGCGTG |
| | | CCCCGCGTGATGCGCGTGCTGCTGCTGGGCGATGTGGCTACCCTGC |
| | | CCCTGCGCAAAGTGCTGGCCGTGGTCGGCACCGCCGAAGCTAGCT |
| | | CCGAACATCCCCTGGGCGTGGCCGTGACCAAATATTGCAAAGAAG |
| | | AACTGGGCACCGAAACCCTGGGCTATTGCACCGATTTTCAGGCCGT |
| | | CCCTGGCTGCGGCATCGGCTGCAAAGTGAGCAATGTGGAGGGCAT |
| | | TCTGGCCCATTCCGAAAGACCCCTGTCCGCCCCTGCTAGCCATCTG |
| | | AATGAAGCCGGCAGCCTGCCTGCTGAGAAAGATGCCGTGCCCCAG |
| | | ACCTTTTCCGTGCTGATCGGCAACCGCGAATGGCTGCGCCGCAACG |
| | | GCCTGACCATTAGCTCCGATGTCTCCGATGCCATGACCGATCATGA |
| | | AATGAAAGGCCAGACCGCCATTCTGGTGGCCATTGACGGCGTGCT |
| | | GTGCGGCATGATTGCCATTGCCGATGCCGTGAAACAGGAGGCTGC |
| | | CCTGGCTGTGCATACCCTGCAGAGCATGGGCGTGGATGTGGTGCTG |
| | | ATTACCGGCGATAACCGCAAAACCGCTCGCGCCATTGCCACCCAG |
| | | GTCGGCATTAACAAAGTGTTTGCTGAAGTGCTGCCCAGCCATAAA |
| | | GTGGCTAAAGTGCAGGAACTGCAGAACAAAGGCAAGAAAGTGGC |
| | | TATGGTCGGCGACGGCGTGAATGATAGCCCTGCCCTGGCCCAGGC |
| | | CGATATGGGCGTGGCTATCGGCACCGGCACCGATGTGGCTATTGA |
| | | GGCTGCCGATGTGGTGCTGATCCGCAATGATCTGCTGGATGTGGTG |
| | | GCTAGCATTCATCTGAGCAAACGCACCGTCCGCCGCATCCGCATTA |
| | | ATCTGGTGCTGGCCCTGATCTATAATCTGGTCGGCATTCCCATCGC |
| | | CGCCGGCGTGTTTATGCCCATCGGCATTGTGCTGCAGCCCTGGATG |
| | | GGCAGCGCCGCTATGGCCGCTAGCTCCGTGTCCGTGGTGCTGAGCA |
| | | GCCTGCAGCTGAAATGCTATAAGAAACCCGATCTGGAAAGATATG |
| | | AAGCTCAGGCTCACGGCCATATGAAACCCCTGACCGCCAGCCAGG |
| | | TGTCCGTGCATATCGGCATGGATGACCGCTGGCGCGATAGCCCCCG |
| | | CGCCACCCCTTGGGATCAGGTGAGCTATGTGAGCCAGGTGTCCCTG |
| | | AGCTCCCTGACCTCCGATAAACCCTCCCGCCATTCCGCCGCTGCCG |
| | | ATGATGACGGCGATAAGTGGAGCCTGCTGCTGAACGGCCGCGATG |
| | | AAGAACAGTATATCTAAGTCCATAAAGTAGGAAACACTACATCAT |
| | | CCATAAAGTAGGAAACACTACATCATCCATAAAGTAGGAAACACT |
| | | ACATCATCCATAAAGTAGGAAACACTACAGAATTCGATATCAAGC |
| | | TTATCGATAATCAACCTCTGGATTACAAAATTTGTGAAAGATTGAC |
| | | TGGTATTCTTAACTATGTTGCTCCTTTTACGCTATGTGGATACGCTG |
| | | CTTTAATGCCTTTGTATCATGCTATTGCTTCCCGTATGGCTTTCATT |
| | | TTCTCCTCCTTGTATAAATCCTGGTTGCTGTCTCTTTATGAGGAGTT |
| | | GTGGCCCGTTGTCAGGCAACGTGGCGTGGTGTGCACTGTGTTTGCT |
| | | GACGCAACCCCCACTGGTTGGGGCACCTGCAGGAATAAAAGATCT |
| | | TTATTTTCATTAGATCTGTGTGTTGGTTTTTTGTGTGTCTAGAGTAG |
| | | ATAAGTAGCATGGCGGGTTAATCATTAACTACAAGGAACCCCTAG |
| | | TGATGGAGTTGGCCACTCCCTCTCTGCGCGCTCGCTCGCTCACTGA |
| | | GGCCGGGCGACCAAAGGTCGCCCGACGCCCGGGCTTTGCCCGGGC |
| | | GGCCTCAGTGAGCGAGCGAGCGCGCAGAGAGGGAGTGGCCAA |
| 102 | A49 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGGTACCGAGTTAATAATTACCAGCGCGGG |
| | | CCAAATAAATAATCCGCGAGGGGCAGCTGGACTTTGGACTCCGCG |
| | | CGCTGGTAATTATTAACCTCGCGAATATTGATTCGAGGCCGCGATT |
| | | GCCGCAATCGCGAGGGGCAGGTGACCTTTGCCCAGCGGGTACCCG |
| | | GGGGCGCGCCAGTCACCCCCTCCACCTTGGACACAGGACGCTGTG |
| | | GTTTCTGAGCCAGGTACAATGACTCCTTTCGGTAAGTGCAGTGGAA |
| | | GCTGTACACTGCCCAGGCAAAGCGTCCGGGCAGCGTAGGCGGGCG |
| | | ACTCAGATCCCAGCCAGTGGACTTAGCCCCTGTTTGCTCCTCCGAT |
| | | AACTGGGGTGACCTTGGTTAATATTCACCAGCAGCCTCCCCCGTTG |
| | | CCCCTCTGGATCCACTGCTTAAATACGGACGAGGACAGGGCCCTGT |
| | | CTCCTCAGCTTCAGGCACCACCACTGACCTGGGACAGTGAATCGGC |
| | | GGCCGCCACCATGCCGGAACAAGAACGCCAGATCACAGCAAGAG |
| | | AAGGAGCAAGTAGGAAGATCCTGAGCAAGCTCTCCCTGCCCACAA |
| | | GGGCCTGGGAGCCGGCCATGAAGAAGTCTTTTGCCTTCGATAATGT |
| | | CGGTTATGAAGGCGGGCTCGATGGCCTGGGCCCCAGCAGCCAGCC |
| | | CCAGAAGTGTTTCCTGCAGATCAAGGGCATGACCTGTGCCAGCTGT |
| | | GTTAGCAATATCGAAAGAAACCTCCAAAAAGAGGCTGGAGTTCTG |
| | | AGTGTTCTAGTGGCTTTAATGGCCGGGAAAGCCGAGATCAAGTAT |
| | | GATCCCGAGGTGATTCAGCCATTGGAGATTGCCCAGTTCATCCAGG |
| | | ATCTGGGCTTCGAGGCCGCCGTGATGGAGGATTACGCCGGCTCTG |
| | | ATGGCAACATCGAACTGACCATCACCGGCATGACCTGTGCTTCCTG |
| | | CGTGCATAATATCGAGAGCAAACTGACGCGGACCAATGGAATCAC |
| | | CTATGCCAGCGTCGCCCTGGCAACCTCTAAGGCTCTGGTGAAGTTT |
| | | GACCCCGAAATCATTGGACCTAGAGACATCATCAAAATTATTGAA |
| | | GAGATTGGTTTCCACGCCAGCTTAGCTCAGCGGAATCCCAACGCAC |
| | | ACCATCTTGACCATAAGATGGAAATCAAACAGTGGAAGAAGAGCT |
| | | TCTTGTGTAGCCTGGTCTTCGGGATACCCGTCATGGCACTGATGAT |
| | | TTACATGCTCATTCCGTCCAACGAGCCCCACCAGTCCATGGTGCTG |
| | | GACCACAACATCATCCCTGGGCTGTCTATCCTCAACCTGATCTTCT |
| | | TTATTCTGTGCACATTTGTCCAGCTCTTGGGAGGCTGGTATTTCTAT |
| | | GTACAGGCCTACAAATCCCTGAGGCATCGAAGCGCCAACATGGAC |
| | | GTTTTAATTGTGCTGGCTACATCCATTGCCTACGTGTACTCCCTGGT |
| | | GATCTTAGTGGTTGCTGTGGCTGAAAAGGCTGAGAGAAGCCCTGT |
| | | CACCTTCTTCGACACCCCCCCCATGCTCTTCGTGTTTATTGCACTGG |
| | | GTCGCTGGCTGGAGCATTTAGCCAAGTCCAAGACATCCGAGGCTCT |
| | | GGCCAAGCTGATGTCTCTACAGGCCACGGAGGCCACCGTGGTGAC |
| | | TCTGGGTGAAGACAATTTGATAATACGCGAGGAACAGGTGCCTAT |
| | | GGAGCTTGTGCAGCGCGGAGATATCGTCAAGGTGGTGCCCGGCGG |
| | | CAAGTTCCCAGTGGATGGAAAAGTGTTAGAGGGCAATACCATGGC |
| | | CGATGAATCCCTCATCACTGGCGAGGCAATGCCGGTGACCAAGAA |
| | | ACCTGGAAGTACTGTCATTGCAGGCAGCATCAATGCCCATGGGAG |
| | | CGTCCTGATCAAGGCCACCCACGTGGGCAATGATACAACTCTCGCC |
| | | CAGATCGTCAAGCTAGTGGAAGAGGCTCAGATGTCTAAAGCTCCA |
| | | ATCCAGCAACTGGCTGACCGCTTCAGCGGCTACTTTGTCCCCTTTA |
| | | TCATCATCATGAGCACCCTCACCCTGGTGGTGTGGATAGTCATAGG |
| | | CTTCATTGACTTTGGCGTGGTGCAGAGATACTTCCCCAACCCTAAC |
| | | AAACATATCTCCCAAACCGAGGTCATTATCAGATTCGCCTTTCAGA |
| | | CATCTATCACTGTGCTGTGCATAGCCTGCCCTTGCTCTCTGGGCCTG |
| | | GCCACCCCCACCGCCGTGATGGTTGGAACCGGGGTCGCTGCACAA |
| | | AATGGGATCCTGATAAAAGGGGGCAAACCCCTGGAAATGGCACAT |
| | | AAGATTAAAACAGTTATGTTTGATAAGACAGGCACCATTACTCAC |
| | | GGTGTCCCTCGAGTCATGAGAGTGCTGCTGCTGGGGGACGTGGCT |
| | | ACACTGCCACTGCGCAAGGTGCTGGCTGTGGTGGGCACAGCGGAG |
| | | GCCTCCTCAGAACACCCATTAGGCGTAGCTGTCACAAAATACTGTA |
| | | AGGAGGAGCTGGGGACAGAGACCCTCGGCTATTGTACAGACTTCC |
| | | AGGCCGTGCCTGGCTGCGGCATCGGCTGTAAGGTCAGCAATGTGG |
| | | AGGGCATCCTGGCACACAGCGAGAGACCCCTCTCTGCCCCAGCCT |
| | | CCCACCTGAATGAAGCTGGGTCTCTGCCTGCTGAGAAGGACGCCG |
| | | TCCCTCAGACCTTCTCCGTGTTGATTGGGAACAGAGAGTGGCTTAG |
| | | AAGGAATGGCCTGACCATCAGCTCTGACGTCTCGGATGCCATGAC |
| | | AGATCACGAGATGAAAGGCCAGACAGCCATACTGGTTGCCATCGA |
| | | CGGCGTGCTCTGTGGCATGATTGCCATCGCTGATGCTGTTAAACAG |
| | | GAAGCTGCGCTGGCAGTCCACACCCTCCAGTCCATGGGAGTGGAC |
| | | GTGGTCCTGATTACTGGAGACAACAGGAAAACCGCTCGGGCCATC |
| | | GCTACCCAAGTGGGGATAAATAAGGTTTTTGCAGAAGTCTTACCCT |
| | | CCCACAAAGTAGCCAAAGTGCAGGAGCTCCAGAACAAGGGTAAA |
| | | AAGGTGGCAATGGTGGGAGATGGTGTGAATGACAGCCCAGCTCTC |
| | | GCGCAGGCTGACATGGGCGTAGCCATAGGTACTGGAACAGATGTC |
| | | GCGATTGAAGCCGCTGACGTAGTCCTCATCCGAAACGATTTGCTGG |
| | | ATGTGGTGGCCAGCATCCATCTGTCCAAGCGTACCGTGCGCCGGAT |
| | | CCGCATCAACCTGGTCTTGGCCCTCATCTATAATCTCGTGGGCATC |
| | | CCCATCGCTGCTGGGGTCTTCATGCCCATCGGCATTGTGCTGCAGC |
| | | CCTGGATGGGAAGCGCTGCCATGGCTGCTTCTTCTGTCTCCGTTGT |
| | | GCTGTCCAGCCTACAGCTGAAGTGCTATAAAAAGCCAGACCTCGA |
| | | GCGCTACGAAGCCCAGGCGCATGGCCATATGAAGCCCCTCACGGC |
| | | TTCCCAGGTCTCGGTTCACATTGGGATGGATGACAGATGGCGGGA |
| | | CTCCCCACGCGCCACACCCTGGGACCAGGTATCCTATGTGAGCCAG |
| | | GTGAGCCTCAGCTCCCTGACATCAGATAAACCGTCTAGGCACTCAG |
| | | CTGCCGCAGATGATGACGGTGACAAATGGAGCCTCCTCCTGAACG |
| | | GCCGCGACGAGGAGCAGTACATCTAAGAATTCGATATCAAGCTTA |
| | | TCGATAATCAACCTCTGGATTACAAAATTTGTGAAAGATTGACTGG |
| | | TATTCTTAACTATGTTGCTCCTTTTACGCTATGTGGATACGCTGCTT |
| | | TAATGCCTTTGTATCATGCTATTGCTTCCCGTATGGCTTTCATTTTC |
| | | TCCTCCTTGTATAAATCCTGGTTGCTGTCTCTTTATGAGGAGTTGTG |
| | | GCCCGTTGTCAGGCAACGTGGCGTGGTGTGCACTGTGTTTGCTGAC |
| | | GCAACCCCCACTGGTTGGGGCACCTGCAGGAATAAAAGATCTTTA |
| | | TTTTCATTAGATCTGTGTGTTGGTTTTTTGTGTGTCTAGAGTAGATA |
| | | AGTAGCATGGCGGGTTAATCATTAACTACAAGGAACCCCTAGTGA |
| | | TGGAGTTGGCCACTCCCTCTCTGCGCGCTCGCTCGCTCACTGAGGC |
| | | CGGGCGACCAAAGGTCGCCCGACGCCCGGGCTTTGCCCGGGCGGC |
| | | CTCAGTGAGCGAGCGAGCGCGCAGAGAGGGAGTGGCCAA |
| 103 | A50 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGGTACCGAGTTAATAATTACCAGCGCGGG |
| | | CCAAATAAATAATCCGCGAGGGGCAGCTGGACTTTGGACTCCGCG |
| | | CGCTGGTAATTATTAACCTCGCGAATATTGATTCGAGGCCGCGATT |
| | | GCCGCAATCGCGAGGGGCAGGTGACCTTTGCCCAGCGGGTACCCG |
| | | GGGGCGCGCCAGTCACCCCCTCCACCTTGGACACAGGACGCTGTG |
| | | GTTTCTGAGCCAGGTACAATGACTCCTTTCGGTAAGTGCAGTGGAA |
| | | GCTGTACACTGCCCAGGCAAAGCGTCCGGGCAGCGTAGGCGGGCG |
| | | ACTCAGATCCCAGCCAGTGGACTTAGCCCCTGTTTGCTCCTCCGAT |
| | | AACTGGGGTGACCTTGGTTAATATTCACCAGCAGCCTCCCCCGTTG |
| | | CCCCTCTGGATCCACTGCTTAAATACGGACGAGGACAGGGCCCTGT |
| | | CTCCTCAGCTTCAGGCACCACCACTGACCTGGGACAGTGAATCGGC |
| | | GGCCGCCACCATGCCCGAACAGGAAAGACAGATTACCGCCCGCGA |
| | | GGGCGCTTCCCGCAAAATTCTGAGCAAACTGAGCCTGCCCACCCG |
| | | CGCCTGGGAACCTGCCATGAAGAAAAGCTTTGCTTTTGATAATGTC |
| | | GGCTATGAGGGCGGCCTGGACGGCCTGGGCCCTAGCAGCCAGGTG |
| | | GCTACCACCCTGATTGCCATTGCCGGCATGACCTGCGCCAGCTGCG |
| | | TGCATAGCATTGAGGGCATGATTAGCCAGCTGGAGGGCGTGCAGC |
| | | AGATTTCCGTGAGCCTGGCTGAGGGCACCGCTACCGTGCTGTATAA |
| | | TCCCTCCGTGATTAGCCCCGAAGAACTGCGCGCTGCCATTGAAGAT |
| | | ATGGGCTTTGAAGCCTCCGTGGTGTCCGAAAGCTGCAGCACCAATC |
| | | CCCTGGGCAATCATTCCGCCGGCAATAGCATGGTGCAGACCACCG |
| | | ACGGCACCCCCACCTCCGTGCAGGAAGTGGCTCCCCATACCGGCC |
| | | GCCTGCCCGCCAATCATGCCCCTGATATTCTGGCTAAAAGCCCCCA |
| | | GAGCACCCGCGCCGTGGCTCCTCAGAAATGCTTTCTGCAGATTAAG |
| | | GGCATGACCTGCGCTAGCTGCGTGAGCAATATTGAACGCAATCTG |
| | | CAGAAAGAAGCCGGCGTGCTGTCCGTGCTGGTGGCCCTGATGGCC |
| | | GGCAAAGCTGAAATCAAATATGATCCCGAAGTGATTCAGCCCCTG |
| | | GAAATTGCCCAGTTCATTCAGGATCTGGGCTTTGAGGCTGCCGTGA |
| | | TGGAAGATTATGCCGGCTCCGACGGCAATATTGAACTGACCATTAC |
| | | CGGCATGACCTGCGCCAGCTGCGTGCATAATATTGAAAGCAAACT |
| | | GACCCGCACCAACGGCATTACCTATGCTTCCGTGGCCCTGGCTACC |
| | | AGCAAAGCCCTGGTGAAATTTGATCCTGAAATTATCGGCCCCCGCG |
| | | ATATTATCAAAATTATTGAAGAAATCGGCTTTCACGCCAGCCTGGC |
| | | TCAGCGCAATCCCAATGCCCATCATCTGGATCATAAGATGGAAATC |
| | | AAACAGTGGAAGAAAAGCTTTCTGTGCAGCCTGGTGTTCGGCATTC |
| | | CTGTGATGGCCCTGATGATCTATATGCTGATTCCCAGCAATGAACC |
| | | CCATCAGAGCATGGTGCTGGATCATAATATTATTCCCGGCCTGAGC |
| | | ATTCTGAATCTGATTTTCTTTATCCTGTGCACCTTTGTGCAGCTGCT |
| | | GGGCGGCTGGTATTTCTATGTGCAGGCTTATAAAAGCCTGCGCCAC |
| | | CGCTCCGCCAATATGGATGTGCTGATTGTGCTGGCCACCAGCATTG |
| | | CTTATGTCTATAGCCTGGTCATTCTGGTGGTGGCCGTGGCCGAGAA |
| | | AGCTGAACGCAGCCCCGTGACCTTCTTTGATACCCCTCCCATGCTG |
| | | TTCGTGTTTATCGCTCTGGGCCGCTGGCTGGAACATCTGGCCAAAA |
| | | GCAAAACCTCCGAAGCCCTGGCTAAACTGATGAGCCTGCAGGCCA |
| | | CCGAAGCCACCGTGGTGACCCTGGGCGAAGATAATCTGATTATCC |
| | | GCGAAGAACAGGTGCCTATGGAACTGGTGCAGCGCGGCGATATTG |
| | | TGAAAGTGGTGCCCGGCGGCAAATTTCCCGTGGACGGCAAAGTGC |
| | | TGGAGGGCAATACCATGGCTGATGAATCCCTGATTACCGGCGAAG |
| | | CTATGCCTGTGACCAAGAAACCCGGCAGCACCGTGATTGCCGGCA |
| | | GCATTAATGCTCACGGCTCCGTGCTGATCAAGGCTACCCATGTCGG |
| | | CAATGATACCACCCTGGCCCAGATTGTGAAACTGGTGGAAGAAGC |
| | | CCAGATGAGCAAAGCCCCTATTCAGCAGCTGGCTGACCGCTTTTCC |
| | | GGCTATTTTGTGCCCTTTATCATTATTATGAGCACCCTGACCCTGGT |
| | | GGTGTGGATTGTGATCGGCTTCATTGATTTCGGCGTGGTGCAGAGA |
| | | TATTTTCCTAATCCCAACAAACATATTAGCCAGACCGAAGTGATTA |
| | | TCCGCTTTGCTTTTCAGACCAGCATTACCGTGCTGTGCATTGCTTGC |
| | | CCCTGCAGCCTGGGCCTGGCTACCCCCACCGCCGTCATGGTCGGCA |
| | | CCGGCGTGGCTGCCCAGAACGGCATTCTGATTAAGGGCGGCAAAC |
| | | CTCTGGAAATGGCCCACAAAATCAAAACCGTGATGTTTGATAAAA |
| | | CCGGCACCATTACCCACGGCGTGCCCCGCGTGATGCGCGTGCTGCT |
| | | GCTGGGCGATGTGGCTACCCTGCCCCTGCGCAAAGTGCTGGCCGTG |
| | | GTCGGCACCGCCGAAGCTAGCTCCGAACATCCCCTGGGCGTGGCC |
| | | GTGACCAAATATTGCAAAGAAGAACTGGGCACCGAAACCCTGGGC |
| | | TATTGCACCGATTTTCAGGCCGTCCCTGGCTGCGGCATCGGCTGCA |
| | | AAGTGAGCAATGTGGAGGGCATTCTGGCCCATTCCGAAAGACCCC |
| | | TGTCCGCCCCTGCTAGCCATCTGAATGAAGCCGGCAGCCTGCCTGC |
| | | TGAGAAAGATGCCGTGCCCCAGACCTTTTCCGTGCTGATCGGCAAC |
| | | CGCGAATGGCTGCGCCGCAACGGCCTGACCATTAGCTCCGATGTCT |
| | | CCGATGCCATGACCGATCATGAAATGAAAGGCCAGACCGCCATTC |
| | | TGGTGGCCATTGACGGCGTGCTGTGCGGCATGATTGCCATTGCCGA |
| | | TGCCGTGAAACAGGAGGCTGCCCTGGCTGTGCATACCCTGCAGAG |
| | | CATGGGCGTGGATGTGGTGCTGATTACCGGCGATAACCGCAAAAC |
| | | CGCTCGCGCCATTGCCACCCAGGTCGGCATTAACAAAGTGTTTGCT |
| | | GAAGTGCTGCCCAGCCATAAAGTGGCTAAAGTGCAGGAACTGCAG |
| | | AACAAAGGCAAGAAAGTGGCTATGGTCGGCGACGGCGTGAATGAT |
| | | AGCCCTGCCCTGGCCCAGGCCGATATGGGCGTGGCTATCGGCACC |
| | | GGCACCGATGTGGCTATTGAGGCTGCCGATGTGGTGCTGATCCGCA |
| | | ATGATCTGCTGGATGTGGTGGCTAGCATTCATCTGAGCAAACGCAC |
| | | CGTCCGCCGCATCCGCATTAATCTGGTGCTGGCCCTGATCTATAAT |
| | | CTGGTCGGCATTCCCATCGCCGCCGGCGTGTTTATGCCCATCGGCA |
| | | TTGTGCTGCAGCCCTGGATGGGCAGCGCCGCTATGGCCGCTAGCTC |
| | | CGTGTCCGTGGTGCTGAGCAGCCTGCAGCTGAAATGCTATAAGAA |
| | | ACCCGATCTGGAAAGATATGAAGCTCAGGCTCACGGCCATATGAA |
| | | ACCCCTGACCGCCAGCCAGGTGTCCGTGCATATCGGCATGGATGA |
| | | CCGCTGGCGCGATAGCCCCCGCGCCACCCCTTGGGATCAGGTGAG |
| | | CTATGTGAGCCAGGTGTCCCTGAGCTCCCTGACCTCCGATAAACCC |
| | | TCCCGCCATTCCGCCGCTGCCGATGATGACGGCGATAAGTGGAGC |
| | | CTGCTGCTGAACGGCCGCGATGAAGAACAGTATATCTAAGTCCAT |
| | | AAAGTAGGAAACACTACATCATCCATAAAGTAGGAAACACTACAT |
| | | CATCCATAAAGTAGGAAACACTACATCATCCATAAAGTAGGAAAC |
| | | ACTACAGAATTCGATATCAAGCTTATCGATAATCAACCTCTGGATT |
| | | ACAAAATTTGTGAAAGATTGACTGGTATTCTTAACTATGTTGCTCC |
| | | TTTTACGCTATGTGGATACGCTGCTTTAATGCCTTTGTATCATGCTA |
| | | TTGCTTCCCGTATGGCTTTCATTTTCTCCTCCTTGTATAAATCCTGG |
| | | TTGCTGTCTCTTTATGAGGAGTTGTGGCCCGTTGTCAGGCAACGTG |
| | | GCGTGGTGTGCACTGTGTTTGCTGACGCAACCCCCACTGGTTGGGG |
| | | CACCTGCAGGAATAAAAGATCTTTATTTTCATTAGATCTGTGTGTT |
| | | GGTTTTTTGTGTGTCTAGAGTAGATAAGTAGCATGGCGGGTTAATC |
| | | ATTAACTACAAGGAACCCCTAGTGATGGAGTTGGCCACTCCCTCTC |
| | | TGCGCGCTCGCTCGCTCACTGAGGCCGGGCGACCAAAGGTCGCCC |
| | | GACGCCCGGGCTTTGCCCGGGCGGCCTCAGTGAGCGAGCGAGCGC |
| | | GCAGAGAGGGAGTGGCCAA |
| 104 | A51 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGGTACCGAGTTAATAATTACCAGCGCGGG |
| | | CCAAATAAATAATCCGCGAGGGGCAGCTGGACTTTGGACTCCGCG |
| | | CGCTGGTAATTATTAACCTCGCGAATATTGATTCGAGGCCGCGATT |
| | | GCCGCAATCGCGAGGGGCAGGTGACCTTTGCCCAGCGCGCGTTCG |
| | | CCCCGCCCCGGACCACCCCCTCCACCTTGGACACAGGACGCTGTGG |
| | | TTTCTGAGCCAGGTACAATGACTCCTTTCGGTAAGTGCAGTGGAAG |
| | | CTGTACACTGCCCAGGCAAAGCGTCCGGGCAGCGTAGGCGGGCGA |
| | | CTCAGATCCCAGCCAGTGGACTTAGCCCCTGTTTGCTCCTCCGATA |
| | | ACTGGGGTGACCTTGGTTAATATTCACCAGCAGCCTCCCCCGTTGC |
| | | CCCTCTGGATCCACTGCTTAAATACGGACGAGGACAGGGCCCTGTC |
| | | TCCTCAGCTTCAGGCACCACCACTGACCTGGGACAGTGAATCGAA |
| | | GAGGTAAGGGTTTAAGGGATGGTTGGTTGGTGGGGTATTAATGTTT |
| | | AATTACCTGGAGCACCTGCCTGAAATCACTTTTTTTCAGGTTGGGC |
| | | GGCCGCCACCATGCCGGAACAAGAACGCCAGATCACAGCAAGAG |
| | | AAGGAGCAAGTAGGAAGATCCTGAGCAAGCTCTCCCTGCCCACAA |
| | | GGGCCTGGGAGCCGGCCATGAAGAAGTCTTTTGCCTTCGATAATGT |
| | | CGGTTATGAAGGCGGGCTCGATGGCCTGGGCCCCAGCAGCCAGCC |
| | | CCAGAAGTGTTTCCTGCAGATCAAGGGCATGACCTGTGCCAGCTGT |
| | | GTTAGCAATATCGAAAGAAACCTCCAAAAAGAGGCTGGAGTTCTG |
| | | AGTGTTCTAGTGGCTTTAATGGCCGGGAAAGCCGAGATCAAGTAT |
| | | GATCCCGAGGTGATTCAGCCATTGGAGATTGCCCAGTTCATCCAGG |
| | | ATCTGGGCTTCGAGGCCGCCGTGATGGAGGATTACGCCGGCTCTG |
| | | ATGGCAACATCGAACTGACCATCACCGGCATGACCTGTGCTTCCTG |
| | | CGTGCATAATATCGAGAGCAAACTGACGCGGACCAATGGAATCAC |
| | | CTATGCCAGCGTCGCCCTGGCAACCTCTAAGGCTCTGGTGAAGTTT |
| | | GACCCCGAAATCATTGGACCTAGAGACATCATCAAAATTATTGAA |
| | | GAGATTGGTTTCCACGCCAGCTTAGCTCAGCGGAATCCCAACGCAC |
| | | ACCATCTTGACCATAAGATGGAAATCAAACAGTGGAAGAAGAGCT |
| | | TCTTGTGTAGCCTGGTCTTCGGGATACCCGTCATGGCACTGATGAT |
| | | TTACATGCTCATTCCGTCCAACGAGCCCCACCAGTCCATGGTGCTG |
| | | GACCACAACATCATCCCTGGGCTGTCTATCCTCAACCTGATCTTCT |
| | | TTATTCTGTGCACATTTGTCCAGCTCTTGGGAGGCTGGTATTTCTAT |
| | | GTACAGGCCTACAAATCCCTGAGGCATCGAAGCGCCAACATGGAC |
| | | GTTTTAATTGTGCTGGCTACATCCATTGCCTACGTGTACTCCCTGGT |
| | | GATCTTAGTGGTTGCTGTGGCTGAAAAGGCTGAGAGAAGCCCTGT |
| | | CACCTTCTTCGACACCCCCCCCATGCTCTTCGTGTTTATTGCACTGG |
| | | GTCGCTGGCTGGAGCATTTAGCCAAGTCCAAGACATCCGAGGCTCT |
| | | GGCCAAGCTGATGTCTCTACAGGCCACGGAGGCCACCGTGGTGAC |
| | | TCTGGGTGAAGACAATTTGATAATACGCGAGGAACAGGTGCCTAT |
| | | GGAGCTTGTGCAGCGCGGAGATATCGTCAAGGTGGTGCCCGGCGG |
| | | CAAGTTCCCAGTGGATGGAAAAGTGTTAGAGGGCAATACCATGGC |
| | | CGATGAATCCCTCATCACTGGCGAGGCAATGCCGGTGACCAAGAA |
| | | ACCTGGAAGTACTGTCATTGCAGGCAGCATCAATGCCCATGGGAG |
| | | CGTCCTGATCAAGGCCACCCACGTGGGCAATGATACAACTCTCGCC |
| | | CAGATCGTCAAGCTAGTGGAAGAGGCTCAGATGTCTAAAGCTCCA |
| | | ATCCAGCAACTGGCTGACCGCTTCAGCGGCTACTTTGTCCCCTTTA |
| | | TCATCATCATGAGCACCCTCACCCTGGTGGTGTGGATAGTCATAGG |
| | | CTTCATTGACTTTGGCGTGGTGCAGAGATACTTCCCCAACCCTAAC |
| | | AAACATATCTCCCAAACCGAGGTCATTATCAGATTCGCCTTTCAGA |
| | | CATCTATCACTGTGCTGTGCATAGCCTGCCCTTGCTCTCTGGGCCTG |
| | | GCCACCCCCACCGCCGTGATGGTTGGAACCGGGGTCGCTGCACAA |
| | | AATGGGATCCTGATAAAAGGGGGCAAACCCCTGGAAATGGCACAT |
| | | AAGATTAAAACAGTTATGTTTGATAAGACAGGCACCATTACTCAC |
| | | GGTGTCCCTCGAGTCATGAGAGTGCTGCTGCTGGGGGACGTGGCT |
| | | ACACTGCCACTGCGCAAGGTGCTGGCTGTGGTGGGCACAGCGGAG |
| | | GCCTCCTCAGAACACCCATTAGGCGTAGCTGTCACAAAATACTGTA |
| | | AGGAGGAGCTGGGGACAGAGACCCTCGGCTATTGTACAGACTTCC |
| | | AGGCCGTGCCTGGCTGCGGCATCGGCTGTAAGGTCAGCAATGTGG |
| | | AGGGCATCCTGGCACACAGCGAGAGACCCCTCTCTGCCCCAGCCT |
| | | CCCACCTGAATGAAGCTGGGTCTCTGCCTGCTGAGAAGGACGCCG |
| | | TCCCTCAGACCTTCTCCGTGTTGATTGGGAACAGAGAGTGGCTTAG |
| | | AAGGAATGGCCTGACCATCAGCTCTGACGTCTCGGATGCCATGAC |
| | | AGATCACGAGATGAAAGGCCAGACAGCCATACTGGTTGCCATCGA |
| | | CGGCGTGCTCTGTGGCATGATTGCCATCGCTGATGCTGTTAAACAG |
| | | GAAGCTGCGCTGGCAGTCCACACCCTCCAGTCCATGGGAGTGGAC |
| | | GTGGTCCTGATTACTGGAGACAACAGGAAAACCGCTCGGGCCATC |
| | | GCTACCCAAGTGGGGATAAATAAGGTTTTTGCAGAAGTCTTACCCT |
| | | CCCACAAAGTAGCCAAAGTGCAGGAGCTCCAGAACAAGGGTAAA |
| | | AAGGTGGCAATGGTGGGAGATGGTGTGAATGACAGCCCAGCTCTC |
| | | GCGCAGGCTGACATGGGCGTAGCCATAGGTACTGGAACAGATGTC |
| | | GCGATTGAAGCCGCTGACGTAGTCCTCATCCGAAACGATTTGCTGG |
| | | ATGTGGTGGCCAGCATCCATCTGTCCAAGCGTACCGTGCGCCGGAT |
| | | CCGCATCAACCTGGTCTTGGCCCTCATCTATAATCTCGTGGGCATC |
| | | CCCATCGCTGCTGGGGTCTTCATGCCCATCGGCATTGTGCTGCAGC |
| | | CCTGGATGGGAAGCGCTGCCATGGCTGCTTCTTCTGTCTCCGTTGT |
| | | GCTGTCCAGCCTACAGCTGAAGTGCTATAAAAAGCCAGACCTCGA |
| | | GCGCTACGAAGCCCAGGCGCATGGCCATATGAAGCCCCTCACGGC |
| | | TTCCCAGGTCTCGGTTCACATTGGGATGGATGACAGATGGCGGGA |
| | | CTCCCCACGCGCCACACCCTGGGACCAGGTATCCTATGTGAGCCAG |
| | | GTGAGCCTCAGCTCCCTGACATCAGATAAACCGTCTAGGCACTCAG |
| | | CTGCCGCAGATGATGACGGTGACAAATGGAGCCTCCTCCTGAACG |
| | | GCCGCGACGAGGAGCAGTACATCTAAGAATTCGATATCAAGCTTA |
| | | TCGATAATCAACCTCTGGATTACAAAATTTGTGAAAGATTGACTGG |
| | | TATTCTTAACTATGTTGCTCCTTTTACGCTATGTGGATACGCTGCTT |
| | | TAATGCCTTTGTATCATGCTATTGCTTCCCGTATGGCTTTCATTTTC |
| | | TCCTCCTTGTATAAATCCTGGTTGCTGTCTCTTTATGAGGAGTTGTG |
| | | GCCCGTTGTCAGGCAACGTGGCGTGGTGTGCACTGTGTTTGCTGAC |
| | | GCAACCCCCACTGGTTGGGGCACCTGCAGGAATAAAAGATCTTTA |
| | | TTTTCATTAGATCTGTGTGTTGGTTTTTTGTGTGTCTAGAGTAGATA |
| | | AGTAGCATGGCGGGTTAATCATTAACTACAAGGAACCCCTAGTGA |
| | | TGGAGTTGGCCACTCCCTCTCTGCGCGCTCGCTCGCTCACTGAGGC |
| | | CGGGCGACCAAAGGTCGCCCGACGCCCGGGCTTTGCCCGGGCGGC |
| | | CTCAGTGAGCGAGCGAGCGCGCAGAGAGGGAGTGGCCAA |
| 105 | A52 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGGTACCGAGTTAATAATTACCAGCGCGGG |
| | | CCAAATAAATAATCCGCGAGGGGCAGCTGGACTTTGGACTCCGCG |
| | | CGCTGGTAATTATTAACCTCGCGAATATTGATTCGAGGCCGCGATT |
| | | GCCGCAATCGCGAGGGGCAGGTGACCTTTGCCCAGCGCGCGTTCG |
| | | CCCCGCCCCGGACCACCCCCTCCACCTTGGACACAGGACGCTGTGG |
| | | TTTCTGAGCCAGGTACAATGACTCCTTTCGGTAAGTGCAGTGGAAG |
| | | CTGTACACTGCCCAGGCAAAGCGTCCGGGCAGCGTAGGCGGGCGA |
| | | CTCAGATCCCAGCCAGTGGACTTAGCCCCTGTTTGCTCCTCCGATA |
| | | ACTGGGGTGACCTTGGTTAATATTCACCAGCAGCCTCCCCCGTTGC |
| | | CCCTCTGGATCCACTGCTTAAATACGGACGAGGACAGGGCCCTGTC |
| | | TCCTCAGCTTCAGGCACCACCACTGACCTGGGACAGTGAATCGAA |
| | | GAGGTAAGGGTTTAAGGGATGGTTGGTTGGTGGGGTATTAATGTTT |
| | | AATTACCTGGAGCACCTGCCTGAAATCACTTTTTTTCAGGTTGGGC |
| | | GGCCGCCACCATGCCCGAACAGGAAAGACAGATTACCGCCCGCGA |
| | | GGGCGCTTCCCGCAAAATTCTGAGCAAACTGAGCCTGCCCACCCG |
| | | CGCCTGGGAACCTGCCATGAAGAAAAGCTTTGCTTTTGATAATGTC |
| | | GGCTATGAGGGCGGCCTGGACGGCCTGGGCCCTAGCAGCCAGGTG |
| | | GCTACCACCCTGATTGCCATTGCCGGCATGACCTGCGCCAGCTGCG |
| | | TGCATAGCATTGAGGGCATGATTAGCCAGCTGGAGGGCGTGCAGC |
| | | AGATTTCCGTGAGCCTGGCTGAGGGCACCGCTACCGTGCTGTATAA |
| | | TCCCTCCGTGATTAGCCCCGAAGAACTGCGCGCTGCCATTGAAGAT |
| | | ATGGGCTTTGAAGCCTCCGTGGTGTCCGAAAGCTGCAGCACCAATC |
| | | CCCTGGGCAATCATTCCGCCGGCAATAGCATGGTGCAGACCACCG |
| | | ACGGCACCCCCACCTCCGTGCAGGAAGTGGCTCCCCATACCGGCC |
| | | GCCTGCCCGCCAATCATGCCCCTGATATTCTGGCTAAAAGCCCCCA |
| | | GAGCACCCGCGCCGTGGCTCCTCAGAAATGCTTTCTGCAGATTAAG |
| | | GGCATGACCTGCGCTAGCTGCGTGAGCAATATTGAACGCAATCTG |
| | | CAGAAAGAAGCCGGCGTGCTGTCCGTGCTGGTGGCCCTGATGGCC |
| | | GGCAAAGCTGAAATCAAATATGATCCCGAAGTGATTCAGCCCCTG |
| | | GAAATTGCCCAGTTCATTCAGGATCTGGGCTTTGAGGCTGCCGTGA |
| | | TGGAAGATTATGCCGGCTCCGACGGCAATATTGAACTGACCATTAC |
| | | CGGCATGACCTGCGCCAGCTGCGTGCATAATATTGAAAGCAAACT |
| | | GACCCGCACCAACGGCATTACCTATGCTTCCGTGGCCCTGGCTACC |
| | | AGCAAAGCCCTGGTGAAATTTGATCCTGAAATTATCGGCCCCCGCG |
| | | ATATTATCAAAATTATTGAAGAAATCGGCTTTCACGCCAGCCTGGC |
| | | TCAGCGCAATCCCAATGCCCATCATCTGGATCATAAGATGGAAATC |
| | | AAACAGTGGAAGAAAAGCTTTCTGTGCAGCCTGGTGTTCGGCATTC |
| | | CTGTGATGGCCCTGATGATCTATATGCTGATTCCCAGCAATGAACC |
| | | CCATCAGAGCATGGTGCTGGATCATAATATTATTCCCGGCCTGAGC |
| | | ATTCTGAATCTGATTTTCTTTATCCTGTGCACCTTTGTGCAGCTGCT |
| | | GGGCGGCTGGTATTTCTATGTGCAGGCTTATAAAAGCCTGCGCCAC |
| | | CGCTCCGCCAATATGGATGTGCTGATTGTGCTGGCCACCAGCATTG |
| | | CTTATGTCTATAGCCTGGTCATTCTGGTGGTGGCCGTGGCCGAGAA |
| | | AGCTGAACGCAGCCCCGTGACCTTCTTTGATACCCCTCCCATGCTG |
| | | TTCGTGTTTATCGCTCTGGGCCGCTGGCTGGAACATCTGGCCAAAA |
| | | GCAAAACCTCCGAAGCCCTGGCTAAACTGATGAGCCTGCAGGCCA |
| | | CCGAAGCCACCGTGGTGACCCTGGGCGAAGATAATCTGATTATCC |
| | | GCGAAGAACAGGTGCCTATGGAACTGGTGCAGCGCGGCGATATTG |
| | | TGAAAGTGGTGCCCGGCGGCAAATTTCCCGTGGACGGCAAAGTGC |
| | | TGGAGGGCAATACCATGGCTGATGAATCCCTGATTACCGGCGAAG |
| | | CTATGCCTGTGACCAAGAAACCCGGCAGCACCGTGATTGCCGGCA |
| | | GCATTAATGCTCACGGCTCCGTGCTGATCAAGGCTACCCATGTCGG |
| | | CAATGATACCACCCTGGCCCAGATTGTGAAACTGGTGGAAGAAGC |
| | | CCAGATGAGCAAAGCCCCTATTCAGCAGCTGGCTGACCGCTTTTCC |
| | | GGCTATTTTGTGCCCTTTATCATTATTATGAGCACCCTGACCCTGGT |
| | | GGTGTGGATTGTGATCGGCTTCATTGATTTCGGCGTGGTGCAGAGA |
| | | TATTTTCCTAATCCCAACAAACATATTAGCCAGACCGAAGTGATTA |
| | | TCCGCTTTGCTTTTCAGACCAGCATTACCGTGCTGTGCATTGCTTGC |
| | | CCCTGCAGCCTGGGCCTGGCTACCCCCACCGCCGTCATGGTCGGCA |
| | | CCGGCGTGGCTGCCCAGAACGGCATTCTGATTAAGGGCGGCAAAC |
| | | CTCTGGAAATGGCCCACAAAATCAAAACCGTGATGTTTGATAAAA |
| | | CCGGCACCATTACCCACGGCGTGCCCCGCGTGATGCGCGTGCTGCT |
| | | GCTGGGCGATGTGGCTACCCTGCCCCTGCGCAAAGTGCTGGCCGTG |
| | | GTCGGCACCGCCGAAGCTAGCTCCGAACATCCCCTGGGCGTGGCC |
| | | GTGACCAAATATTGCAAAGAAGAACTGGGCACCGAAACCCTGGGC |
| | | TATTGCACCGATTTTCAGGCCGTCCCTGGCTGCGGCATCGGCTGCA |
| | | AAGTGAGCAATGTGGAGGGCATTCTGGCCCATTCCGAAAGACCCC |
| | | TGTCCGCCCCTGCTAGCCATCTGAATGAAGCCGGCAGCCTGCCTGC |
| | | TGAGAAAGATGCCGTGCCCCAGACCTTTTCCGTGCTGATCGGCAAC |
| | | CGCGAATGGCTGCGCCGCAACGGCCTGACCATTAGCTCCGATGTCT |
| | | CCGATGCCATGACCGATCATGAAATGAAAGGCCAGACCGCCATTC |
| | | TGGTGGCCATTGACGGCGTGCTGTGCGGCATGATTGCCATTGCCGA |
| | | TGCCGTGAAACAGGAGGCTGCCCTGGCTGTGCATACCCTGCAGAG |
| | | CATGGGCGTGGATGTGGTGCTGATTACCGGCGATAACCGCAAAAC |
| | | CGCTCGCGCCATTGCCACCCAGGTCGGCATTAACAAAGTGTTTGCT |
| | | GAAGTGCTGCCCAGCCATAAAGTGGCTAAAGTGCAGGAACTGCAG |
| | | AACAAAGGCAAGAAAGTGGCTATGGTCGGCGACGGCGTGAATGAT |
| | | AGCCCTGCCCTGGCCCAGGCCGATATGGGCGTGGCTATCGGCACC |
| | | GGCACCGATGTGGCTATTGAGGCTGCCGATGTGGTGCTGATCCGCA |
| | | ATGATCTGCTGGATGTGGTGGCTAGCATTCATCTGAGCAAACGCAC |
| | | CGTCCGCCGCATCCGCATTAATCTGGTGCTGGCCCTGATCTATAAT |
| | | CTGGTCGGCATTCCCATCGCCGCCGGCGTGTTTATGCCCATCGGCA |
| | | TTGTGCTGCAGCCCTGGATGGGCAGCGCCGCTATGGCCGCTAGCTC |
| | | CGTGTCCGTGGTGCTGAGCAGCCTGCAGCTGAAATGCTATAAGAA |
| | | ACCCGATCTGGAAAGATATGAAGCTCAGGCTCACGGCCATATGAA |
| | | ACCCCTGACCGCCAGCCAGGTGTCCGTGCATATCGGCATGGATGA |
| | | CCGCTGGCGCGATAGCCCCCGCGCCACCCCTTGGGATCAGGTGAG |
| | | CTATGTGAGCCAGGTGTCCCTGAGCTCCCTGACCTCCGATAAACCC |
| | | TCCCGCCATTCCGCCGCTGCCGATGATGACGGCGATAAGTGGAGC |
| | | CTGCTGCTGAACGGCCGCGATGAAGAACAGTATATCTAAGTCCAT |
| | | AAAGTAGGAAACACTACATCATCCATAAAGTAGGAAACACTACAT |
| | | CATCCATAAAGTAGGAAACACTACATCATCCATAAAGTAGGAAAC |
| | | ACTACAGAATTCGATATCAAGCTTATCGATAATCAACCTCTGGATT |
| | | ACAAAATTTGTGAAAGATTGACTGGTATTCTTAACTATGTTGCTCC |
| | | TTTTACGCTATGTGGATACGCTGCTTTAATGCCTTTGTATCATGCTA |
| | | TTGCTTCCCGTATGGCTTTCATTTTCTCCTCCTTGTATAAATCCTGG |
| | | TTGCTGTCTCTTTATGAGGAGTTGTGGCCCGTTGTCAGGCAACGTG |
| | | GCGTGGTGTGCACTGTGTTTGCTGACGCAACCCCCACTGGTTGGGG |
| | | CACCTGCAGGAATAAAAGATCTTTATTTTCATTAGATCTGTGTGTT |
| | | GGTTTTTTGTGTGTCTAGAGTAGATAAGTAGCATGGCGGGTTAATC |
| | | ATTAACTACAAGGAACCCCTAGTGATGGAGTTGGCCACTCCCTCTC |
| | | TGCGCGCTCGCTCGCTCACTGAGGCCGGGCGACCAAAGGTCGCCC |
| | | GACGCCCGGGCTTTGCCCGGGCGGCCTCAGTGAGCGAGCGAGCGC |
| | | GCAGAGAGGGAGTGGCCAA |
| 106 | A53 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGACCCTAAAATGGGCAAACATTGCAAGCAGC |
| | | AAACAGCAAACACACAGCCCTCCCTGCCTGCTGACCTTGGAGCTG |
| | | GGGCAGAGGTCAGAGACCTCTCTGGGCCCATGCCACCTCCAACAT |
| | | CCACTCGACCCCTTGGAATTTCGGTGGAGAGGAGCAGAGGTTGTC |
| | | CTGGCGTGGTTTAGGTAGTGTGAGAGGGGAATGACTCCTTTCGGTA |
| | | AGTGCAGTGGAAGCTGTACACTGCCCAGGCAAAGCGTCCGGGCAG |
| | | CGTAGGCGGGCGACTCAGATCCCAGCCAGTGGACTTAGCCCCTGTT |
| | | TGCTCCTCCGATAACTGGGGTGACCTTGGTTAATATTCACCAGCAG |
| | | CCTCCCCCGTTGCCCCTCTGGATCCACTGCTTAAATACGGACGAGG |
| | | ACAGGGCCCTGTCTCCTCAGCTTCAGGCACCACCACTGACCTGGGA |
| | | CAGTGAATCGGCGGCCGCCACCATGCCCGAACAGGAAAGACAGAT |
| | | TACCGCCCGCGAGGGCGCTTCCCGCAAAATTCTGAGCAAACTGAG |
| | | CCTGCCCACCCGCGCCTGGGAACCTGCCATGAAGAAAAGCTTTGCT |
| | | TTTGATAATGTCGGCTATGAGGGCGGCCTGGACGGCCTGGGCCCTA |
| | | GCAGCCAGGTGGCTACCACCCTGATTGCCATTGCCGGCATGACCTG |
| | | CGCCAGCTGCGTGCATAGCATTGAGGGCATGATTAGCCAGCTGGA |
| | | GGGCGTGCAGCAGATTTCCGTGAGCCTGGCTGAGGGCACCGCTAC |
| | | CGTGCTGTATAATCCCTCCGTGATTAGCCCCGAAGAACTGCGCGCT |
| | | GCCATTGAAGATATGGGCTTTGAAGCCTCCGTGGTGTCCGAAAGCT |
| | | GCAGCACCAATCCCCTGGGCAATCATTCCGCCGGCAATAGCATGG |
| | | TGCAGACCACCGACGGCACCCCCACCTCCGTGCAGGAAGTGGCTC |
| | | CCCATACCGGCCGCCTGCCCGCCAATCATGCCCCTGATATTCTGGC |
| | | TAAAAGCCCCCAGAGCACCCGCGCCGTGGCTCCTCAGAAATGCTTT |
| | | CTGCAGATTAAGGGCATGACCTGCGCTAGCTGCGTGAGCAATATT |
| | | GAACGCAATCTGCAGAAAGAAGCCGGCGTGCTGTCCGTGCTGGTG |
| | | GCCCTGATGGCCGGCAAAGCTGAAATCAAATATGATCCCGAAGTG |
| | | ATTCAGCCCCTGGAAATTGCCCAGTTCATTCAGGATCTGGGCTTTG |
| | | AGGCTGCCGTGATGGAAGATTATGCCGGCTCCGACGGCAATATTG |
| | | AACTGACCATTACCGGCATGACCTGCGCCAGCTGCGTGCATAATAT |
| | | TGAAAGCAAACTGACCCGCACCAACGGCATTACCTATGCTTCCGTG |
| | | GCCCTGGCTACCAGCAAAGCCCTGGTGAAATTTGATCCTGAAATTA |
| | | TCGGCCCCCGCGATATTATCAAAATTATTGAAGAAATCGGCTTTCA |
| | | CGCCAGCCTGGCTCAGCGCAATCCCAATGCCCATCATCTGGATCAT |
| | | AAGATGGAAATCAAACAGTGGAAGAAAAGCTTTCTGTGCAGCCTG |
| | | GTGTTCGGCATTCCTGTGATGGCCCTGATGATCTATATGCTGATTC |
| | | CCAGCAATGAACCCCATCAGAGCATGGTGCTGGATCATAATATTAT |
| | | TCCCGGCCTGAGCATTCTGAATCTGATTTTCTTTATCCTGTGCACCT |
| | | TTGTGCAGCTGCTGGGCGGCTGGTATTTCTATGTGCAGGCTTATAA |
| | | AAGCCTGCGCCACCGCTCCGCCAATATGGATGTGCTGATTGTGCTG |
| | | GCCACCAGCATTGCTTATGTCTATAGCCTGGTCATTCTGGTGGTGG |
| | | CCGTGGCCGAGAAAGCTGAACGCAGCCCCGTGACCTTCTTTGATAC |
| | | CCCTCCCATGCTGTTCGTGTTTATCGCTCTGGGCCGCTGGCTGGAA |
| | | CATCTGGCCAAAAGCAAAACCTCCGAAGCCCTGGCTAAACTGATG |
| | | AGCCTGCAGGCCACCGAAGCCACCGTGGTGACCCTGGGCGAAGAT |
| | | AATCTGATTATCCGCGAAGAACAGGTGCCTATGGAACTGGTGCAG |
| | | CGCGGCGATATTGTGAAAGTGGTGCCCGGCGGCAAATTTCCCGTG |
| | | GACGGCAAAGTGCTGGAGGGCAATACCATGGCTGATGAATCCCTG |
| | | ATTACCGGCGAAGCTATGCCTGTGACCAAGAAACCCGGCAGCACC |
| | | GTGATTGCCGGCAGCATTAATGCTCACGGCTCCGTGCTGATCAAGG |
| | | CTACCCATGTCGGCAATGATACCACCCTGGCCCAGATTGTGAAACT |
| | | GGTGGAAGAAGCCCAGATGAGCAAAGCCCCTATTCAGCAGCTGGC |
| | | TGACCGCTTTTCCGGCTATTTTGTGCCCTTTATCATTATTATGAGCA |
| | | CCCTGACCCTGGTGGTGTGGATTGTGATCGGCTTCATTGATTTCGG |
| | | CGTGGTGCAGAGATATTTTCCTAATCCCAACAAACATATTAGCCAG |
| | | ACCGAAGTGATTATCCGCTTTGCTTTTCAGACCAGCATTACCGTGC |
| | | TGTGCATTGCTTGCCCCTGCAGCCTGGGCCTGGCTACCCCCACCGC |
| | | CGTCATGGTCGGCACCGGCGTGGCTGCCCAGAACGGCATTCTGATT |
| | | AAGGGCGGCAAACCTCTGGAAATGGCCCACAAAATCAAAACCGTG |
| | | ATGTTTGATAAAACCGGCACCATTACCCACGGCGTGCCCCGCGTGA |
| | | TGCGCGTGCTGCTGCTGGGCGATGTGGCTACCCTGCCCCTGCGCAA |
| | | AGTGCTGGCCGTGGTCGGCACCGCCGAAGCTAGCTCCGAACATCC |
| | | CCTGGGCGTGGCCGTGACCAAATATTGCAAAGAAGAACTGGGCAC |
| | | CGAAACCCTGGGCTATTGCACCGATTTTCAGGCCGTCCCTGGCTGC |
| | | GGCATCGGCTGCAAAGTGAGCAATGTGGAGGGCATTCTGGCCCAT |
| | | TCCGAAAGACCCCTGTCCGCCCCTGCTAGCCATCTGAATGAAGCCG |
| | | GCAGCCTGCCTGCTGAGAAAGATGCCGTGCCCCAGACCTTTTCCGT |
| | | GCTGATCGGCAACCGCGAATGGCTGCGCCGCAACGGCCTGACCAT |
| | | TAGCTCCGATGTCTCCGATGCCATGACCGATCATGAAATGAAAGG |
| | | CCAGACCGCCATTCTGGTGGCCATTGACGGCGTGCTGTGCGGCATG |
| | | ATTGCCATTGCCGATGCCGTGAAACAGGAGGCTGCCCTGGCTGTGC |
| | | ATACCCTGCAGAGCATGGGCGTGGATGTGGTGCTGATTACCGGCG |
| | | ATAACCGCAAAACCGCTCGCGCCATTGCCACCCAGGTCGGCATTA |
| | | ACAAAGTGTTTGCTGAAGTGCTGCCCAGCCATAAAGTGGCTAAAG |
| | | TGCAGGAACTGCAGAACAAAGGCAAGAAAGTGGCTATGGTCGGCG |
| | | ACGGCGTGAATGATAGCCCTGCCCTGGCCCAGGCCGATATGGGCG |
| | | TGGCTATCGGCACCGGCACCGATGTGGCTATTGAGGCTGCCGATGT |
| | | GGTGCTGATCCGCAATGATCTGCTGGATGTGGTGGCTAGCATTCAT |
| | | CTGAGCAAACGCACCGTCCGCCGCATCCGCATTAATCTGGTGCTGG |
| | | CCCTGATCTATAATCTGGTCGGCATTCCCATCGCCGCCGGCGTGTT |
| | | TATGCCCATCGGCATTGTGCTGCAGCCCTGGATGGGCAGCGCCGCT |
| | | ATGGCCGCTAGCTCCGTGTCCGTGGTGCTGAGCAGCCTGCAGCTGA |
| | | AATGCTATAAGAAACCCGATCTGGAAAGATATGAAGCTCAGGCTC |
| | | ACGGCCATATGAAACCCCTGACCGCCAGCCAGGTGTCCGTGCATA |
| | | TCGGCATGGATGACCGCTGGCGCGATAGCCCCCGCGCCACCCCTTG |
| | | GGATCAGGTGAGCTATGTGAGCCAGGTGTCCCTGAGCTCCCTGACC |
| | | TCCGATAAACCCTCCCGCCATTCCGCCGCTGCCGATGATGACGGCG |
| | | ATAAGTGGAGCCTGCTGCTGAACGGCCGCGATGAAGAACAGTATA |
| | | TCTAAGTCCATAAAGTAGGAAACACTACATCATCCATAAAGTAGG |
| | | AAACACTACATCATCCATAAAGTAGGAAACACTACATCATCCATA |
| | | AAGTAGGAAACACTACAGAATTCGATATCAAGCTTATCGATAATC |
| | | AACCTCTGGATTACAAAATTTGTGAAAGATTGACTGGTATTCTTAA |
| | | CTATGTTGCTCCTTTTACGCTATGTGGATACGCTGCTTTAATGCCTT |
| | | TGTATCATGCTATTGCTTCCCGTATGGCTTTCATTTTCTCCTCCTTGT |
| | | ATAAATCCTGGTTGCTGTCTCTTTATGAGGAGTTGTGGCCCGTTGT |
| | | CAGGCAACGTGGCGTGGTGTGCACTGTGTTTGCTGACGCAACCCCC |
| | | ACTGGTTGGGGCACCTGCAGGAATAAAAGATCTTTATTTTCATTAG |
| | | ATCTGTGTGTTGGTTTTTTGTGTGTCTAGAGTAGATAAGTAGCATG |
| | | GCGGGTTAATCATTAACTACAAGGAACCCCTAGTGATGGAGTTGG |
| | | CCACTCCCTCTCTGCGCGCTCGCTCGCTCACTGAGGCCGGGCGACC |
| | | AAAGGTCGCCCGACGCCCGGGCTTTGCCCGGGCGGCCTCAGTGAG |
| | | CGAGCGAGCGCGCAGAGAGGGAGTGGCCAA |
| 107 | A54 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGACCCTAAAATGGGCAAACATTGCAAGCAGC |
| | | AAACAGCAAACACACAGCCCTCCCTGCCTGCTGACCTTGGAGCTG |
| | | GGGCAGAGGTCAGAGACCTCTCTGGGCCCATGCCACCTCCAACAT |
| | | CCACTCGACCCCTTGGAATTTCGGTGGAGAGGAGCAGAGGTTGTC |
| | | CTGGCGTGGTTTAGGTAGTGTGAGAGGGGAATGACTCCTTTCGGTA |
| | | AGTGCAGTGGAAGCTGTACACTGCCCAGGCAAAGCGTCCGGGCAG |
| | | CGTAGGCGGGCGACTCAGATCCCAGCCAGTGGACTTAGCCCCTGTT |
| | | TGCTCCTCCGATAACTGGGGTGACCTTGGTTAATATTCACCAGCAG |
| | | CCTCCCCCGTTGCCCCTCTGGATCCACTGCTTAAATACGGACGAGG |
| | | ACAGGGCCCTGTCTCCTCAGCTTCAGGCACCACCACTGACCTGGGA |
| | | CAGTGAATCGGCGGCCGCCACCATGCCCGAACAGGAAAGACAGAT |
| | | TACCGCCCGCGAGGGCGCTTCCCGCAAAATTCTGAGCAAACTGAG |
| | | CCTGCCCACCCGCGCCTGGGAACCTGCCATGAAGAAAAGCTTTGCT |
| | | TTTGATAATGTCGGCTATGAGGGCGGCCTGGACGGCCTGGGCCCTA |
| | | GCAGCCAGGTGGCTACCACCCTGATTGCCATTGCCGGCATGACCTG |
| | | CGCCAGCTGCGTGCATAGCATTGAGGGCATGATTAGCCAGCTGGA |
| | | GGGCGTGCAGCAGATTTCCGTGAGCCTGGCTGAGGGCACCGCTAC |
| | | CGTGCTGTATAATCCCTCCGTGATTAGCCCCGAAGAACTGCGCGCT |
| | | GCCATTGAAGATATGGGCTTTGAAGCCTCCGTGGTGTCCGAAAGCT |
| | | GCAGCACCAATCCCCTGGGCAATCATTCCGCCGGCAATAGCATGG |
| | | TGCAGACCACCGACGGCACCCCCACCTCCGTGCAGGAAGTGGCTC |
| | | CCCATACCGGCCGCCTGCCCGCCAATCATGCCCCTGATATTCTGGC |
| | | TAAAAGCCCCCAGAGCACCCGCGCCGTGGCTCCTCAGAAATGCTTT |
| | | CTGCAGATTAAGGGCATGACCTGCGCTAGCTGCGTGAGCAATATT |
| | | GAACGCAATCTGCAGAAAGAAGCCGGCGTGCTGTCCGTGCTGGTG |
| | | GCCCTGATGGCCGGCAAAGCTGAAATCAAATATGATCCCGAAGTG |
| | | ATTCAGCCCCTGGAAATTGCCCAGTTCATTCAGGATCTGGGCTTTG |
| | | AGGCTGCCGTGATGGAAGATTATGCCGGCTCCGACGGCAATATTG |
| | | AACTGACCATTACCGGCATGACCTGCGCCAGCTGCGTGCATAATAT |
| | | TGAAAGCAAACTGACCCGCACCAACGGCATTACCTATGCTTCCGTG |
| | | GCCCTGGCTACCAGCAAAGCCCTGGTGAAATTTGATCCTGAAATTA |
| | | TCGGCCCCCGCGATATTATCAAAATTATTGAAGAAATCGGCTTTCA |
| | | CGCCAGCCTGGCTCAGCGCAATCCCAATGCCCATCATCTGGATCAT |
| | | AAGATGGAAATCAAACAGTGGAAGAAAAGCTTTCTGTGCAGCCTG |
| | | GTGTTCGGCATTCCTGTGATGGCCCTGATGATCTATATGCTGATTC |
| | | CCAGCAATGAACCCCATCAGAGCATGGTGCTGGATCATAATATTAT |
| | | TCCCGGCCTGAGCATTCTGAATCTGATTTTCTTTATCCTGTGCACCT |
| | | TTGTGCAGCTGCTGGGCGGCTGGTATTTCTATGTGCAGGCTTATAA |
| | | AAGCCTGCGCCACCGCTCCGCCAATATGGATGTGCTGATTGTGCTG |
| | | GCCACCAGCATTGCTTATGTCTATAGCCTGGTCATTCTGGTGGTGG |
| | | CCGTGGCCGAGAAAGCTGAACGCAGCCCCGTGACCTTCTTTGATAC |
| | | CCCTCCCATGCTGTTCGTGTTTATCGCTCTGGGCCGCTGGCTGGAA |
| | | CATCTGGCCAAAAGCAAAACCTCCGAAGCCCTGGCTAAACTGATG |
| | | AGCCTGCAGGCCACCGAAGCCACCGTGGTGACCCTGGGCGAAGAT |
| | | AATCTGATTATCCGCGAAGAACAGGTGCCTATGGAACTGGTGCAG |
| | | CGCGGCGATATTGTGAAAGTGGTGCCCGGCGGCAAATTTCCCGTG |
| | | GACGGCAAAGTGCTGGAGGGCAATACCATGGCTGATGAATCCCTG |
| | | ATTACCGGCGAAGCTATGCCTGTGACCAAGAAACCCGGCAGCACC |
| | | GTGATTGCCGGCAGCATTAATGCTCACGGCTCCGTGCTGATCAAGG |
| | | CTACCCATGTCGGCAATGATACCACCCTGGCCCAGATTGTGAAACT |
| | | GGTGGAAGAAGCCCAGATGAGCAAAGCCCCTATTCAGCAGCTGGC |
| | | TGACCGCTTTTCCGGCTATTTTGTGCCCTTTATCATTATTATGAGCA |
| | | CCCTGACCCTGGTGGTGTGGATTGTGATCGGCTTCATTGATTTCGG |
| | | CGTGGTGCAGAGATATTTTCCTAATCCCAACAAACATATTAGCCAG |
| | | ACCGAAGTGATTATCCGCTTTGCTTTTCAGACCAGCATTACCGTGC |
| | | TGTGCATTGCTTGCCCCTGCAGCCTGGGCCTGGCTACCCCCACCGC |
| | | CGTCATGGTCGGCACCGGCGTGGCTGCCCAGAACGGCATTCTGATT |
| | | AAGGGCGGCAAACCTCTGGAAATGGCCCACAAAATCAAAACCGTG |
| | | ATGTTTGATAAAACCGGCACCATTACCCACGGCGTGCCCCGCGTGA |
| | | TGCGCGTGCTGCTGCTGGGCGATGTGGCTACCCTGCCCCTGCGCAA |
| | | AGTGCTGGCCGTGGTCGGCACCGCCGAAGCTAGCTCCGAACATCC |
| | | CCTGGGCGTGGCCGTGACCAAATATTGCAAAGAAGAACTGGGCAC |
| | | CGAAACCCTGGGCTATTGCACCGATTTTCAGGCCGTCCCTGGCTGC |
| | | GGCATCGGCTGCAAAGTGAGCAATGTGGAGGGCATTCTGGCCCAT |
| | | TCCGAAAGACCCCTGTCCGCCCCTGCTAGCCATCTGAATGAAGCCG |
| | | GCAGCCTGCCTGCTGAGAAAGATGCCGTGCCCCAGACCTTTTCCGT |
| | | GCTGATCGGCAACCGCGAATGGCTGCGCCGCAACGGCCTGACCAT |
| | | TAGCTCCGATGTCTCCGATGCCATGACCGATCATGAAATGAAAGG |
| | | CCAGACCGCCATTCTGGTGGCCATTGACGGCGTGCTGTGCGGCATG |
| | | ATTGCCATTGCCGATGCCGTGAAACAGGAGGCTGCCCTGGCTGTGC |
| | | ATACCCTGCAGAGCATGGGCGTGGATGTGGTGCTGATTACCGGCG |
| | | ATAACCGCAAAACCGCTCGCGCCATTGCCACCCAGGTCGGCATTA |
| | | ACAAAGTGTTTGCTGAAGTGCTGCCCAGCCATAAAGTGGCTAAAG |
| | | TGCAGGAACTGCAGAACAAAGGCAAGAAAGTGGCTATGGTCGGCG |
| | | ACGGCGTGAATGATAGCCCTGCCCTGGCCCAGGCCGATATGGGCG |
| | | TGGCTATCGGCACCGGCACCGATGTGGCTATTGAGGCTGCCGATGT |
| | | GGTGCTGATCCGCAATGATCTGCTGGATGTGGTGGCTAGCATTCAT |
| | | CTGAGCAAACGCACCGTCCGCCGCATCCGCATTAATCTGGTGCTGG |
| | | CCCTGATCTATAATCTGGTCGGCATTCCCATCGCCGCCGGCGTGTT |
| | | TATGCCCATCGGCATTGTGCTGCAGCCCTGGATGGGCAGCGCCGCT |
| | | ATGGCCGCTAGCTCCGTGTCCGTGGTGCTGAGCAGCCTGCAGCTGA |
| | | AATGCTATAAGAAACCCGATCTGGAAAGATATGAAGCTCAGGCTC |
| | | ACGGCCATATGAAACCCCTGACCGCCAGCCAGGTGTCCGTGCATA |
| | | TCGGCATGGATGACCGCTGGCGCGATAGCCCCCGCGCCACCCCTTG |
| | | GGATCAGGTGAGCTATGTGAGCCAGGTGTCCCTGAGCTCCCTGACC |
| | | TCCGATAAACCCTCCCGCCATTCCGCCGCTGCCGATGATGACGGCG |
| | | ATAAGTGGAGCCTGCTGCTGAACGGCCGCGATGAAGAACAGTATA |
| | | TCTAAGTCCATAAAGTAGGAAACACTACATCATCCATAAAGTAGG |
| | | AAACACTACATCATCCATAAAGTAGGAAACACTACATCATCCATA |
| | | AAGTAGGAAACACTACAGAATTCGATATCAAGCTTATCGATAATC |
| | | AACCTCTGGATTACAAAATTTGTGAAAGATTGACTGGTATTCTTAA |
| | | CTATGTTGCTCCTTTTACGCTATGTGGATACGCTGCTTTAATGCCTT |
| | | TGTATCATGCTATTGCTTCCCGTATGGCTTTCATTTTCTCCTCCTTGT |
| | | ATAAATCCTGGTTGCTGTCTCTTTATGAGGAGTTGTGGCCCGTTGT |
| | | CAGGCAACGTGGCGTGGTGTGCACTGTGTTTGCTGACGCAACCCCC |
| | | ACTGGTTGGGGCACCTGCAGGAATAAAAGATCTTTATTTTCATTAG |
| | | ATCTGTGTGTTGGTTTTTTGTGTGTCTAGAGTAGATAAGTAGCATG |
| | | GCGGGTTAATCATTAACTACAAGGAACCCCTAGTGATGGAGTTGG |
| | | CCACTCCCTCTCTGCGCGCTCGCTCGCTCACTGAGGCCGGGCGACC |
| | | AAAGGTCGCCCGACGCCCGGGCTTTGCCCGGGCGGCCTCAGTGAG |
| | | CGAGCGAGCGCGCAGAGAGGGAGTGGCCAA |
| 108 | A55 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGGTACCGAGTTAATAATTACCAGCGCGGG |
| | | CCAAATAAATAATCCGCGAGGGGCAGCTGGACTTTGGACTCCGCG |
| | | CGCTGGTAATTATTAACCTCCCTAAAATGGGCAAACATTGCAAGCA |
| | | GCAAACAGCAAACACACAGCCCTCCCTGCCTGCTGACCTTGGAGC |
| | | TGGGGCAGAGGTCAGAGACCTCTCTGGGCCCATGCCACCTCCAAC |
| | | ATCCACTCGACCCCTTGGAATTTCGGTGGAGAGGAGCAGAGGTTG |
| | | TCCTGGCGTGGTTTAGGTAGTGTGAGAGGGGAATGACTCCTTTCGG |
| | | TAAGTGCAGTGGAAGCTGTACACTGCCCAGGCAAAGCGTCCGGGC |
| | | AGCGTAGGCGGGCGACTCAGATCCCAGCCAGTGGACTTAGCCCCT |
| | | GTTTGCTCCTCCGATAACTGGGGTGACCTTGGTTAATATTCACCAG |
| | | CAGCCTCCCCCGTTGCCCCTCTGGATCCACTGCTTAAATACGGACG |
| | | AGGACAGGGCCCTGTCTCCTCAGCTTCAGGCACCACCACTGACCTG |
| | | GGACAGTGAATCGGCGGCCGCCACCATGCCGGAACAAGAACGCCA |
| | | GATCACAGCAAGAGAAGGAGCAAGTAGGAAGATCCTGAGCAAGC |
| | | TCTCCCTGCCCACAAGGGCCTGGGAGCCGGCCATGAAGAAGTCTTT |
| | | TGCCTTCGATAATGTCGGTTATGAAGGCGGGCTCGATGGCCTGGGC |
| | | CCCAGCAGCCAGCCCCAGAAGTGTTTCCTGCAGATCAAGGGCATG |
| | | ACCTGTGCCAGCTGTGTTAGCAATATCGAAAGAAACCTCCAAAAA |
| | | GAGGCTGGAGTTCTGAGTGTTCTAGTGGCTTTAATGGCCGGGAAA |
| | | GCCGAGATCAAGTATGATCCCGAGGTGATTCAGCCATTGGAGATT |
| | | GCCCAGTTCATCCAGGATCTGGGCTTCGAGGCCGCCGTGATGGAG |
| | | GATTACGCCGGCTCTGATGGCAACATCGAACTGACCATCACCGGC |
| | | ATGACCTGTGCTTCCTGCGTGCATAATATCGAGAGCAAACTGACGC |
| | | GGACCAATGGAATCACCTATGCCAGCGTCGCCCTGGCAACCTCTA |
| | | AGGCTCTGGTGAAGTTTGACCCCGAAATCATTGGACCTAGAGACA |
| | | TCATCAAAATTATTGAAGAGATTGGTTTCCACGCCAGCTTAGCTCA |
| | | GCGGAATCCCAACGCACACCATCTTGACCATAAGATGGAAATCAA |
| | | ACAGTGGAAGAAGAGCTTCTTGTGTAGCCTGGTCTTCGGGATACCC |
| | | GTCATGGCACTGATGATTTACATGCTCATTCCGTCCAACGAGCCCC |
| | | ACCAGTCCATGGTGCTGGACCACAACATCATCCCTGGGCTGTCTAT |
| | | CCTCAACCTGATCTTCTTTATTCTGTGCACATTTGTCCAGCTCTTGG |
| | | GAGGCTGGTATTTCTATGTACAGGCCTACAAATCCCTGAGGCATCG |
| | | AAGCGCCAACATGGACGTTTTAATTGTGCTGGCTACATCCATTGCC |
| | | TACGTGTACTCCCTGGTGATCTTAGTGGTTGCTGTGGCTGAAAAGG |
| | | CTGAGAGAAGCCCTGTCACCTTCTTCGACACCCCCCCCATGCTCTT |
| | | CGTGTTTATTGCACTGGGTCGCTGGCTGGAGCATTTAGCCAAGTCC |
| | | AAGACATCCGAGGCTCTGGCCAAGCTGATGTCTCTACAGGCCACG |
| | | GAGGCCACCGTGGTGACTCTGGGTGAAGACAATTTGATAATACGC |
| | | GAGGAACAGGTGCCTATGGAGCTTGTGCAGCGCGGAGATATCGTC |
| | | AAGGTGGTGCCCGGCGGCAAGTTCCCAGTGGATGGAAAAGTGTTA |
| | | GAGGGCAATACCATGGCCGATGAATCCCTCATCACTGGCGAGGCA |
| | | ATGCCGGTGACCAAGAAACCTGGAAGTACTGTCATTGCAGGCAGC |
| | | ATCAATGCCCATGGGAGCGTCCTGATCAAGGCCACCCACGTGGGC |
| | | AATGATACAACTCTCGCCCAGATCGTCAAGCTAGTGGAAGAGGCT |
| | | CAGATGTCTAAAGCTCCAATCCAGCAACTGGCTGACCGCTTCAGCG |
| | | GCTACTTTGTCCCCTTTATCATCATCATGAGCACCCTCACCCTGGTG |
| | | GTGTGGATAGTCATAGGCTTCATTGACTTTGGCGTGGTGCAGAGAT |
| | | ACTTCCCCAACCCTAACAAACATATCTCCCAAACCGAGGTCATTAT |
| | | CAGATTCGCCTTTCAGACATCTATCACTGTGCTGTGCATAGCCTGC |
| | | CCTTGCTCTCTGGGCCTGGCCACCCCCACCGCCGTGATGGTTGGAA |
| | | CCGGGGTCGCTGCACAAAATGGGATCCTGATAAAAGGGGGCAAAC |
| | | CCCTGGAAATGGCACATAAGATTAAAACAGTTATGTTTGATAAGA |
| | | CAGGCACCATTACTCACGGTGTCCCTCGAGTCATGAGAGTGCTGCT |
| | | GCTGGGGGACGTGGCTACACTGCCACTGCGCAAGGTGCTGGCTGT |
| | | GGTGGGCACAGCGGAGGCCTCCTCAGAACACCCATTAGGCGTAGC |
| | | TGTCACAAAATACTGTAAGGAGGAGCTGGGGACAGAGACCCTCGG |
| | | CTATTGTACAGACTTCCAGGCCGTGCCTGGCTGCGGCATCGGCTGT |
| | | AAGGTCAGCAATGTGGAGGGCATCCTGGCACACAGCGAGAGACCC |
| | | CTCTCTGCCCCAGCCTCCCACCTGAATGAAGCTGGGTCTCTGCCTG |
| | | CTGAGAAGGACGCCGTCCCTCAGACCTTCTCCGTGTTGATTGGGAA |
| | | CAGAGAGTGGCTTAGAAGGAATGGCCTGACCATCAGCTCTGACGT |
| | | CTCGGATGCCATGACAGATCACGAGATGAAAGGCCAGACAGCCAT |
| | | ACTGGTTGCCATCGACGGCGTGCTCTGTGGCATGATTGCCATCGCT |
| | | GATGCTGTTAAACAGGAAGCTGCGCTGGCAGTCCACACCCTCCAG |
| | | TCCATGGGAGTGGACGTGGTCCTGATTACTGGAGACAACAGGAAA |
| | | ACCGCTCGGGCCATCGCTACCCAAGTGGGGATAAATAAGGTTTTTG |
| | | CAGAAGTCTTACCCTCCCACAAAGTAGCCAAAGTGCAGGAGCTCC |
| | | AGAACAAGGGTAAAAAGGTGGCAATGGTGGGAGATGGTGTGAAT |
| | | GACAGCCCAGCTCTCGCGCAGGCTGACATGGGCGTAGCCATAGGT |
| | | ACTGGAACAGATGTCGCGATTGAAGCCGCTGACGTAGTCCTCATCC |
| | | GAAACGATTTGCTGGATGTGGTGGCCAGCATCCATCTGTCCAAGCG |
| | | TACCGTGCGCCGGATCCGCATCAACCTGGTCTTGGCCCTCATCTAT |
| | | AATCTCGTGGGCATCCCCATCGCTGCTGGGGTCTTCATGCCCATCG |
| | | GCATTGTGCTGCAGCCCTGGATGGGAAGCGCTGCCATGGCTGCTTC |
| | | TTCTGTCTCCGTTGTGCTGTCCAGCCTACAGCTGAAGTGCTATAAA |
| | | AAGCCAGACCTCGAGCGCTACGAAGCCCAGGCGCATGGCCATATG |
| | | AAGCCCCTCACGGCTTCCCAGGTCTCGGTTCACATTGGGATGGATG |
| | | ACAGATGGCGGGACTCCCCACGCGCCACACCCTGGGACCAGGTAT |
| | | CCTATGTGAGCCAGGTGAGCCTCAGCTCCCTGACATCAGATAAACC |
| | | GTCTAGGCACTCAGCTGCCGCAGATGATGACGGTGACAAATGGAG |
| | | CCTCCTCCTGAACGGCCGCGACGAGGAGCAGTACATCTAAGAATT |
| | | CGATATCAAGCTTATCGATAATCAACCTCTGGATTACAAAATTTGT |
| | | GAAAGATTGACTGGTATTCTTAACTATGTTGCTCCTTTTACGCTATG |
| | | TGGATACGCTGCTTTAATGCCTTTGTATCATGCTATTGCTTCCCGTA |
| | | TGGCTTTCATTTTCTCCTCCTTGTATAAATCCTGGTTGCTGTCTCTTT |
| | | ATGAGGAGTTGTGGCCCGTTGTCAGGCAACGTGGCGTGGTGTGCA |
| | | CTGTGTTTGCTGACGCAACCCCCACTGGTTGGGGCACCTGCAGGAA |
| | | TAAAAGATCTTTATTTTCATTAGATCTGTGTGTTGGTTTTTTGTGTG |
| | | TCTAGAGTAGATAAGTAGCATGGCGGGTTAATCATTAACTACAAG |
| | | GAACCCCTAGTGATGGAGTTGGCCACTCCCTCTCTGCGCGCTCGCT |
| | | CGCTCACTGAGGCCGGGCGACCAAAGGTCGCCCGACGCCCGGGCT |
| | | TTGCCCGGGCGGCCTCAGTGAGCGAGCGAGCGCGCAGAGAGGGAG |
| | | TGGCCAA |
| 109 | A56 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGGTACCGAGTTAATAATTACCAGCGCGGG |
| | | CCAAATAAATAATCCGCGAGGGGCAGCTGGACTTTGGACTCCGCG |
| | | CGCTGGTAATTATTAACCTCCCTAAAATGGGCAAACATTGCAAGCA |
| | | GCAAACAGCAAACACACAGCCCTCCCTGCCTGCTGACCTTGGAGC |
| | | TGGGGCAGAGGTCAGAGACCTCTCTGGGCCCATGCCACCTCCAAC |
| | | ATCCACTCGACCCCTTGGAATTTCGGTGGAGAGGAGCAGAGGTTG |
| | | TCCTGGCGTGGTTTAGGTAGTGTGAGAGGGGAATGACTCCTTTCGG |
| | | TAAGTGCAGTGGAAGCTGTACACTGCCCAGGCAAAGCGTCCGGGC |
| | | AGCGTAGGCGGGCGACTCAGATCCCAGCCAGTGGACTTAGCCCCT |
| | | GTTTGCTCCTCCGATAACTGGGGTGACCTTGGTTAATATTCACCAG |
| | | CAGCCTCCCCCGTTGCCCCTCTGGATCCACTGCTTAAATACGGACG |
| | | AGGACAGGGCCCTGTCTCCTCAGCTTCAGGCACCACCACTGACCTG |
| | | GGACAGTGAATCGGCGGCCGCCACCATGCCCGAACAGGAAAGACA |
| | | GATTACCGCCCGCGAGGGCGCTTCCCGCAAAATTCTGAGCAAACT |
| | | GAGCCTGCCCACCCGCGCCTGGGAACCTGCCATGAAGAAAAGCTT |
| | | TGCTTTTGATAATGTCGGCTATGAGGGCGGCCTGGACGGCCTGGGC |
| | | CCTAGCAGCCAGGTGGCTACCACCCTGATTGCCATTGCCGGCATGA |
| | | CCTGCGCCAGCTGCGTGCATAGCATTGAGGGCATGATTAGCCAGCT |
| | | GGAGGGCGTGCAGCAGATTTCCGTGAGCCTGGCTGAGGGCACCGC |
| | | TACCGTGCTGTATAATCCCTCCGTGATTAGCCCCGAAGAACTGCGC |
| | | GCTGCCATTGAAGATATGGGCTTTGAAGCCTCCGTGGTGTCCGAAA |
| | | GCTGCAGCACCAATCCCCTGGGCAATCATTCCGCCGGCAATAGCAT |
| | | GGTGCAGACCACCGACGGCACCCCCACCTCCGTGCAGGAAGTGGC |
| | | TCCCCATACCGGCCGCCTGCCCGCCAATCATGCCCCTGATATTCTG |
| | | GCTAAAAGCCCCCAGAGCACCCGCGCCGTGGCTCCTCAGAAATGC |
| | | TTTCTGCAGATTAAGGGCATGACCTGCGCTAGCTGCGTGAGCAATA |
| | | TTGAACGCAATCTGCAGAAAGAAGCCGGCGTGCTGTCCGTGCTGG |
| | | TGGCCCTGATGGCCGGCAAAGCTGAAATCAAATATGATCCCGAAG |
| | | TGATTCAGCCCCTGGAAATTGCCCAGTTCATTCAGGATCTGGGCTT |
| | | TGAGGCTGCCGTGATGGAAGATTATGCCGGCTCCGACGGCAATAT |
| | | TGAACTGACCATTACCGGCATGACCTGCGCCAGCTGCGTGCATAAT |
| | | ATTGAAAGCAAACTGACCCGCACCAACGGCATTACCTATGCTTCCG |
| | | TGGCCCTGGCTACCAGCAAAGCCCTGGTGAAATTTGATCCTGAAAT |
| | | TATCGGCCCCCGCGATATTATCAAAATTATTGAAGAAATCGGCTTT |
| | | CACGCCAGCCTGGCTCAGCGCAATCCCAATGCCCATCATCTGGATC |
| | | ATAAGATGGAAATCAAACAGTGGAAGAAAAGCTTTCTGTGCAGCC |
| | | TGGTGTTCGGCATTCCTGTGATGGCCCTGATGATCTATATGCTGAT |
| | | TCCCAGCAATGAACCCCATCAGAGCATGGTGCTGGATCATAATATT |
| | | ATTCCCGGCCTGAGCATTCTGAATCTGATTTTCTTTATCCTGTGCAC |
| | | CTTTGTGCAGCTGCTGGGCGGCTGGTATTTCTATGTGCAGGCTTAT |
| | | AAAAGCCTGCGCCACCGCTCCGCCAATATGGATGTGCTGATTGTGC |
| | | TGGCCACCAGCATTGCTTATGTCTATAGCCTGGTCATTCTGGTGGT |
| | | GGCCGTGGCCGAGAAAGCTGAACGCAGCCCCGTGACCTTCTTTGA |
| | | TACCCCTCCCATGCTGTTCGTGTTTATCGCTCTGGGCCGCTGGCTGG |
| | | AACATCTGGCCAAAAGCAAAACCTCCGAAGCCCTGGCTAAACTGA |
| | | TGAGCCTGCAGGCCACCGAAGCCACCGTGGTGACCCTGGGCGAAG |
| | | ATAATCTGATTATCCGCGAAGAACAGGTGCCTATGGAACTGGTGC |
| | | AGCGCGGCGATATTGTGAAAGTGGTGCCCGGCGGCAAATTTCCCG |
| | | TGGACGGCAAAGTGCTGGAGGGCAATACCATGGCTGATGAATCCC |
| | | TGATTACCGGCGAAGCTATGCCTGTGACCAAGAAACCCGGCAGCA |
| | | CCGTGATTGCCGGCAGCATTAATGCTCACGGCTCCGTGCTGATCAA |
| | | GGCTACCCATGTCGGCAATGATACCACCCTGGCCCAGATTGTGAA |
| | | ACTGGTGGAAGAAGCCCAGATGAGCAAAGCCCCTATTCAGCAGCT |
| | | GGCTGACCGCTTTTCCGGCTATTTTGTGCCCTTTATCATTATTATGA |
| | | GCACCCTGACCCTGGTGGTGTGGATTGTGATCGGCTTCATTGATTT |
| | | CGGCGTGGTGCAGAGATATTTTCCTAATCCCAACAAACATATTAGC |
| | | CAGACCGAAGTGATTATCCGCTTTGCTTTTCAGACCAGCATTACCG |
| | | TGCTGTGCATTGCTTGCCCCTGCAGCCTGGGCCTGGCTACCCCCAC |
| | | CGCCGTCATGGTCGGCACCGGCGTGGCTGCCCAGAACGGCATTCT |
| | | GATTAAGGGCGGCAAACCTCTGGAAATGGCCCACAAAATCAAAAC |
| | | CGTGATGTTTGATAAAACCGGCACCATTACCCACGGCGTGCCCCGC |
| | | GTGATGCGCGTGCTGCTGCTGGGCGATGTGGCTACCCTGCCCCTGC |
| | | GCAAAGTGCTGGCCGTGGTCGGCACCGCCGAAGCTAGCTCCGAAC |
| | | ATCCCCTGGGCGTGGCCGTGACCAAATATTGCAAAGAAGAACTGG |
| | | GCACCGAAACCCTGGGCTATTGCACCGATTTTCAGGCCGTCCCTGG |
| | | CTGCGGCATCGGCTGCAAAGTGAGCAATGTGGAGGGCATTCTGGC |
| | | CCATTCCGAAAGACCCCTGTCCGCCCCTGCTAGCCATCTGAATGAA |
| | | GCCGGCAGCCTGCCTGCTGAGAAAGATGCCGTGCCCCAGACCTTTT |
| | | CCGTGCTGATCGGCAACCGCGAATGGCTGCGCCGCAACGGCCTGA |
| | | CCATTAGCTCCGATGTCTCCGATGCCATGACCGATCATGAAATGAA |
| | | AGGCCAGACCGCCATTCTGGTGGCCATTGACGGCGTGCTGTGCGG |
| | | CATGATTGCCATTGCCGATGCCGTGAAACAGGAGGCTGCCCTGGCT |
| | | GTGCATACCCTGCAGAGCATGGGCGTGGATGTGGTGCTGATTACC |
| | | GGCGATAACCGCAAAACCGCTCGCGCCATTGCCACCCAGGTCGGC |
| | | ATTAACAAAGTGTTTGCTGAAGTGCTGCCCAGCCATAAAGTGGCTA |
| | | AAGTGCAGGAACTGCAGAACAAAGGCAAGAAAGTGGCTATGGTC |
| | | GGCGACGGCGTGAATGATAGCCCTGCCCTGGCCCAGGCCGATATG |
| | | GGCGTGGCTATCGGCACCGGCACCGATGTGGCTATTGAGGCTGCC |
| | | GATGTGGTGCTGATCCGCAATGATCTGCTGGATGTGGTGGCTAGCA |
| | | TTCATCTGAGCAAACGCACCGTCCGCCGCATCCGCATTAATCTGGT |
| | | GCTGGCCCTGATCTATAATCTGGTCGGCATTCCCATCGCCGCCGGC |
| | | GTGTTTATGCCCATCGGCATTGTGCTGCAGCCCTGGATGGGCAGCG |
| | | CCGCTATGGCCGCTAGCTCCGTGTCCGTGGTGCTGAGCAGCCTGCA |
| | | GCTGAAATGCTATAAGAAACCCGATCTGGAAAGATATGAAGCTCA |
| | | GGCTCACGGCCATATGAAACCCCTGACCGCCAGCCAGGTGTCCGT |
| | | GCATATCGGCATGGATGACCGCTGGCGCGATAGCCCCCGCGCCAC |
| | | CCCTTGGGATCAGGTGAGCTATGTGAGCCAGGTGTCCCTGAGCTCC |
| | | CTGACCTCCGATAAACCCTCCCGCCATTCCGCCGCTGCCGATGATG |
| | | ACGGCGATAAGTGGAGCCTGCTGCTGAACGGCCGCGATGAAGAAC |
| | | AGTATATCTAAGTCCATAAAGTAGGAAACACTACATCATCCATAA |
| | | AGTAGGAAACACTACATCATCCATAAAGTAGGAAACACTACATCA |
| | | TCCATAAAGTAGGAAACACTACAGAATTCGATATCAAGCTTATCG |
| | | ATAATCAACCTCTGGATTACAAAATTTGTGAAAGATTGACTGGTAT |
| | | TCTTAACTATGTTGCTCCTTTTACGCTATGTGGATACGCTGCTTTAA |
| | | TGCCTTTGTATCATGCTATTGCTTCCCGTATGGCTTTCATTTTCTCCT |
| | | CCTTGTATAAATCCTGGTTGCTGTCTCTTTATGAGGAGTTGTGGCCC |
| | | GTTGTCAGGCAACGTGGCGTGGTGTGCACTGTGTTTGCTGACGCAA |
| | | CCCCCACTGGTTGGGGCACCTGCAGGAATAAAAGATCTTTATTTTC |
| | | ATTAGATCTGTGTGTTGGTTTTTTGTGTGTCTAGAGTAGATAAGTA |
| | | GCATGGCGGGTTAATCATTAACTACAAGGAACCCCTAGTGATGGA |
| | | GTTGGCCACTCCCTCTCTGCGCGCTCGCTCGCTCACTGAGGCCGGG |
| | | CGACCAAAGGTCGCCCGACGCCCGGGCTTTGCCCGGGCGGCCTCA |
| | | GTGAGCGAGCGAGCGCGCAGAGAGGGAGTGGCCAA |
| 110 | A57 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGGTACCGAGTTAATAATTACCAGCGCGGG |
| | | CCAAATAAATAATCCGCGAGGGGCAGCTGGACTTTGGACTCCGCG |
| | | CGCTGGTAATTATTAACCTCGCGAATATTGATTCGAGGCCGCGATT |
| | | GCCGCAATCGCGAGGGGCAGGTGACCTTTGCCCAGCGGGTACCCG |
| | | GGGGCGCGCCAGTCCCTAAAATGGGCAAACATTGCAAGCAGCAAA |
| | | CAGCAAACACACAGCCCTCCCTGCCTGCTGACCTTGGAGCTGGGG |
| | | CAGAGGTCAGAGACCTCTCTGGGCCCATGCCACCTCCAACATCCAC |
| | | TCGACCCCTTGGAATTTCGGTGGAGAGGAGCAGAGGTTGTCCTGG |
| | | CGTGGTTTAGGTAGTGTGAGAGGGGAATGACTCCTTTCGGTAAGTG |
| | | CAGTGGAAGCTGTACACTGCCCAGGCAAAGCGTCCGGGCAGCGTA |
| | | GGCGGGCGACTCAGATCCCAGCCAGTGGACTTAGCCCCTGTTTGCT |
| | | CCTCCGATAACTGGGGTGACCTTGGTTAATATTCACCAGCAGCCTC |
| | | CCCCGTTGCCCCTCTGGATCCACTGCTTAAATACGGACGAGGACAG |
| | | GGCCCTGTCTCCTCAGCTTCAGGCACCACCACTGACCTGGGACAGT |
| | | GAATCGGCGGCCGCCACCATGCCGGAACAAGAACGCCAGATCACA |
| | | GCAAGAGAAGGAGCAAGTAGGAAGATCCTGAGCAAGCTCTCCCTG |
| | | CCCACAAGGGCCTGGGAGCCGGCCATGAAGAAGTCTTTTGCCTTC |
| | | GATAATGTCGGTTATGAAGGCGGGCTCGATGGCCTGGGCCCCAGC |
| | | AGCCAGCCCCAGAAGTGTTTCCTGCAGATCAAGGGCATGACCTGT |
| | | GCCAGCTGTGTTAGCAATATCGAAAGAAACCTCCAAAAAGAGGCT |
| | | GGAGTTCTGAGTGTTCTAGTGGCTTTAATGGCCGGGAAAGCCGAG |
| | | ATCAAGTATGATCCCGAGGTGATTCAGCCATTGGAGATTGCCCAGT |
| | | TCATCCAGGATCTGGGCTTCGAGGCCGCCGTGATGGAGGATTACG |
| | | CCGGCTCTGATGGCAACATCGAACTGACCATCACCGGCATGACCT |
| | | GTGCTTCCTGCGTGCATAATATCGAGAGCAAACTGACGCGGACCA |
| | | ATGGAATCACCTATGCCAGCGTCGCCCTGGCAACCTCTAAGGCTCT |
| | | GGTGAAGTTTGACCCCGAAATCATTGGACCTAGAGACATCATCAA |
| | | AATTATTGAAGAGATTGGTTTCCACGCCAGCTTAGCTCAGCGGAAT |
| | | CCCAACGCACACCATCTTGACCATAAGATGGAAATCAAACAGTGG |
| | | AAGAAGAGCTTCTTGTGTAGCCTGGTCTTCGGGATACCCGTCATGG |
| | | CACTGATGATTTACATGCTCATTCCGTCCAACGAGCCCCACCAGTC |
| | | CATGGTGCTGGACCACAACATCATCCCTGGGCTGTCTATCCTCAAC |
| | | CTGATCTTCTTTATTCTGTGCACATTTGTCCAGCTCTTGGGAGGCTG |
| | | GTATTTCTATGTACAGGCCTACAAATCCCTGAGGCATCGAAGCGCC |
| | | AACATGGACGTTTTAATTGTGCTGGCTACATCCATTGCCTACGTGT |
| | | ACTCCCTGGTGATCTTAGTGGTTGCTGTGGCTGAAAAGGCTGAGAG |
| | | AAGCCCTGTCACCTTCTTCGACACCCCCCCCATGCTCTTCGTGTTTA |
| | | TTGCACTGGGTCGCTGGCTGGAGCATTTAGCCAAGTCCAAGACATC |
| | | CGAGGCTCTGGCCAAGCTGATGTCTCTACAGGCCACGGAGGCCAC |
| | | CGTGGTGACTCTGGGTGAAGACAATTTGATAATACGCGAGGAACA |
| | | GGTGCCTATGGAGCTTGTGCAGCGCGGAGATATCGTCAAGGTGGT |
| | | GCCCGGCGGCAAGTTCCCAGTGGATGGAAAAGTGTTAGAGGGCAA |
| | | TACCATGGCCGATGAATCCCTCATCACTGGCGAGGCAATGCCGGT |
| | | GACCAAGAAACCTGGAAGTACTGTCATTGCAGGCAGCATCAATGC |
| | | CCATGGGAGCGTCCTGATCAAGGCCACCCACGTGGGCAATGATAC |
| | | AACTCTCGCCCAGATCGTCAAGCTAGTGGAAGAGGCTCAGATGTC |
| | | TAAAGCTCCAATCCAGCAACTGGCTGACCGCTTCAGCGGCTACTTT |
| | | GTCCCCTTTATCATCATCATGAGCACCCTCACCCTGGTGGTGTGGA |
| | | TAGTCATAGGCTTCATTGACTTTGGCGTGGTGCAGAGATACTTCCC |
| | | CAACCCTAACAAACATATCTCCCAAACCGAGGTCATTATCAGATTC |
| | | GCCTTTCAGACATCTATCACTGTGCTGTGCATAGCCTGCCCTTGCTC |
| | | TCTGGGCCTGGCCACCCCCACCGCCGTGATGGTTGGAACCGGGGTC |
| | | GCTGCACAAAATGGGATCCTGATAAAAGGGGGCAAACCCCTGGAA |
| | | ATGGCACATAAGATTAAAACAGTTATGTTTGATAAGACAGGCACC |
| | | ATTACTCACGGTGTCCCTCGAGTCATGAGAGTGCTGCTGCTGGGGG |
| | | ACGTGGCTACACTGCCACTGCGCAAGGTGCTGGCTGTGGTGGGCA |
| | | CAGCGGAGGCCTCCTCAGAACACCCATTAGGCGTAGCTGTCACAA |
| | | AATACTGTAAGGAGGAGCTGGGGACAGAGACCCTCGGCTATTGTA |
| | | CAGACTTCCAGGCCGTGCCTGGCTGCGGCATCGGCTGTAAGGTCA |
| | | GCAATGTGGAGGGCATCCTGGCACACAGCGAGAGACCCCTCTCTG |
| | | CCCCAGCCTCCCACCTGAATGAAGCTGGGTCTCTGCCTGCTGAGAA |
| | | GGACGCCGTCCCTCAGACCTTCTCCGTGTTGATTGGGAACAGAGAG |
| | | TGGCTTAGAAGGAATGGCCTGACCATCAGCTCTGACGTCTCGGATG |
| | | CCATGACAGATCACGAGATGAAAGGCCAGACAGCCATACTGGTTG |
| | | CCATCGACGGCGTGCTCTGTGGCATGATTGCCATCGCTGATGCTGT |
| | | TAAACAGGAAGCTGCGCTGGCAGTCCACACCCTCCAGTCCATGGG |
| | | AGTGGACGTGGTCCTGATTACTGGAGACAACAGGAAAACCGCTCG |
| | | GGCCATCGCTACCCAAGTGGGGATAAATAAGGTTTTTGCAGAAGT |
| | | CTTACCCTCCCACAAAGTAGCCAAAGTGCAGGAGCTCCAGAACAA |
| | | GGGTAAAAAGGTGGCAATGGTGGGAGATGGTGTGAATGACAGCCC |
| | | AGCTCTCGCGCAGGCTGACATGGGCGTAGCCATAGGTACTGGAAC |
| | | AGATGTCGCGATTGAAGCCGCTGACGTAGTCCTCATCCGAAACGA |
| | | TTTGCTGGATGTGGTGGCCAGCATCCATCTGTCCAAGCGTACCGTG |
| | | CGCCGGATCCGCATCAACCTGGTCTTGGCCCTCATCTATAATCTCG |
| | | TGGGCATCCCCATCGCTGCTGGGGTCTTCATGCCCATCGGCATTGT |
| | | GCTGCAGCCCTGGATGGGAAGCGCTGCCATGGCTGCTTCTTCTGTC |
| | | TCCGTTGTGCTGTCCAGCCTACAGCTGAAGTGCTATAAAAAGCCAG |
| | | ACCTCGAGCGCTACGAAGCCCAGGCGCATGGCCATATGAAGCCCC |
| | | TCACGGCTTCCCAGGTCTCGGTTCACATTGGGATGGATGACAGATG |
| | | GCGGGACTCCCCACGCGCCACACCCTGGGACCAGGTATCCTATGT |
| | | GAGCCAGGTGAGCCTCAGCTCCCTGACATCAGATAAACCGTCTAG |
| | | GCACTCAGCTGCCGCAGATGATGACGGTGACAAATGGAGCCTCCT |
| | | CCTGAACGGCCGCGACGAGGAGCAGTACATCTAAGAATTCGATAT |
| | | CAAGCTTATCGATAATCAACCTCTGGATTACAAAATTTGTGAAAGA |
| | | TTGACTGGTATTCTTAACTATGTTGCTCCTTTTACGCTATGTGGATA |
| | | CGCTGCTTTAATGCCTTTGTATCATGCTATTGCTTCCCGTATGGCTT |
| | | TCATTTTCTCCTCCTTGTATAAATCCTGGTTGCTGTCTCTTTATGAG |
| | | GAGTTGTGGCCCGTTGTCAGGCAACGTGGCGTGGTGTGCACTGTGT |
| | | TTGCTGACGCAACCCCCACTGGTTGGGGCACCTGCAGGAATAAAA |
| | | GATCTTTATTTTCATTAGATCTGTGTGTTGGTTTTTTGTGTGTCTAG |
| | | AGTAGATAAGTAGCATGGCGGGTTAATCATTAACTACAAGGAACC |
| | | CCTAGTGATGGAGTTGGCCACTCCCTCTCTGCGCGCTCGCTCGCTC |
| | | ACTGAGGCCGGGCGACCAAAGGTCGCCCGACGCCCGGGCTTTGCC |
| | | CGGGCGGCCTCAGTGAGCGAGCGAGCGCGCAGAGAGGGAGTGGC |
| | | CAA |
| 111 | A58 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGGTACCGAGTTAATAATTACCAGCGCGGG |
| | | CCAAATAAATAATCCGCGAGGGGCAGCTGGACTTTGGACTCCGCG |
| | | CGCTGGTAATTATTAACCTCGCGAATATTGATTCGAGGCCGCGATT |
| | | GCCGCAATCGCGAGGGGCAGGTGACCTTTGCCCAGCGGGTACCCG |
| | | GGGGCGCGCCAGTCCCTAAAATGGGCAAACATTGCAAGCAGCAAA |
| | | CAGCAAACACACAGCCCTCCCTGCCTGCTGACCTTGGAGCTGGGG |
| | | CAGAGGTCAGAGACCTCTCTGGGCCCATGCCACCTCCAACATCCAC |
| | | TCGACCCCTTGGAATTTCGGTGGAGAGGAGCAGAGGTTGTCCTGG |
| | | CGTGGTTTAGGTAGTGTGAGAGGGGAATGACTCCTTTCGGTAAGTG |
| | | CAGTGGAAGCTGTACACTGCCCAGGCAAAGCGTCCGGGCAGCGTA |
| | | GGCGGGCGACTCAGATCCCAGCCAGTGGACTTAGCCCCTGTTTGCT |
| | | CCTCCGATAACTGGGGTGACCTTGGTTAATATTCACCAGCAGCCTC |
| | | CCCCGTTGCCCCTCTGGATCCACTGCTTAAATACGGACGAGGACAG |
| | | GGCCCTGTCTCCTCAGCTTCAGGCACCACCACTGACCTGGGACAGT |
| | | GAATCGGCGGCCGCCACCATGCCCGAACAGGAAAGACAGATTACC |
| | | GCCCGCGAGGGCGCTTCCCGCAAAATTCTGAGCAAACTGAGCCTG |
| | | CCCACCCGCGCCTGGGAACCTGCCATGAAGAAAAGCTTTGCTTTTG |
| | | ATAATGTCGGCTATGAGGGCGGCCTGGACGGCCTGGGCCCTAGCA |
| | | GCCAGGTGGCTACCACCCTGATTGCCATTGCCGGCATGACCTGCGC |
| | | CAGCTGCGTGCATAGCATTGAGGGCATGATTAGCCAGCTGGAGGG |
| | | CGTGCAGCAGATTTCCGTGAGCCTGGCTGAGGGCACCGCTACCGT |
| | | GCTGTATAATCCCTCCGTGATTAGCCCCGAAGAACTGCGCGCTGCC |
| | | ATTGAAGATATGGGCTTTGAAGCCTCCGTGGTGTCCGAAAGCTGCA |
| | | GCACCAATCCCCTGGGCAATCATTCCGCCGGCAATAGCATGGTGC |
| | | AGACCACCGACGGCACCCCCACCTCCGTGCAGGAAGTGGCTCCCC |
| | | ATACCGGCCGCCTGCCCGCCAATCATGCCCCTGATATTCTGGCTAA |
| | | AAGCCCCCAGAGCACCCGCGCCGTGGCTCCTCAGAAATGCTTTCTG |
| | | CAGATTAAGGGCATGACCTGCGCTAGCTGCGTGAGCAATATTGAA |
| | | CGCAATCTGCAGAAAGAAGCCGGCGTGCTGTCCGTGCTGGTGGCC |
| | | CTGATGGCCGGCAAAGCTGAAATCAAATATGATCCCGAAGTGATT |
| | | CAGCCCCTGGAAATTGCCCAGTTCATTCAGGATCTGGGCTTTGAGG |
| | | CTGCCGTGATGGAAGATTATGCCGGCTCCGACGGCAATATTGAACT |
| | | GACCATTACCGGCATGACCTGCGCCAGCTGCGTGCATAATATTGAA |
| | | AGCAAACTGACCCGCACCAACGGCATTACCTATGCTTCCGTGGCCC |
| | | TGGCTACCAGCAAAGCCCTGGTGAAATTTGATCCTGAAATTATCGG |
| | | CCCCCGCGATATTATCAAAATTATTGAAGAAATCGGCTTTCACGCC |
| | | AGCCTGGCTCAGCGCAATCCCAATGCCCATCATCTGGATCATAAGA |
| | | TGGAAATCAAACAGTGGAAGAAAAGCTTTCTGTGCAGCCTGGTGT |
| | | TCGGCATTCCTGTGATGGCCCTGATGATCTATATGCTGATTCCCAG |
| | | CAATGAACCCCATCAGAGCATGGTGCTGGATCATAATATTATTCCC |
| | | GGCCTGAGCATTCTGAATCTGATTTTCTTTATCCTGTGCACCTTTGT |
| | | GCAGCTGCTGGGCGGCTGGTATTTCTATGTGCAGGCTTATAAAAGC |
| | | CTGCGCCACCGCTCCGCCAATATGGATGTGCTGATTGTGCTGGCCA |
| | | CCAGCATTGCTTATGTCTATAGCCTGGTCATTCTGGTGGTGGCCGT |
| | | GGCCGAGAAAGCTGAACGCAGCCCCGTGACCTTCTTTGATACCCCT |
| | | CCCATGCTGTTCGTGTTTATCGCTCTGGGCCGCTGGCTGGAACATC |
| | | TGGCCAAAAGCAAAACCTCCGAAGCCCTGGCTAAACTGATGAGCC |
| | | TGCAGGCCACCGAAGCCACCGTGGTGACCCTGGGCGAAGATAATC |
| | | TGATTATCCGCGAAGAACAGGTGCCTATGGAACTGGTGCAGCGCG |
| | | GCGATATTGTGAAAGTGGTGCCCGGCGGCAAATTTCCCGTGGACG |
| | | GCAAAGTGCTGGAGGGCAATACCATGGCTGATGAATCCCTGATTA |
| | | CCGGCGAAGCTATGCCTGTGACCAAGAAACCCGGCAGCACCGTGA |
| | | TTGCCGGCAGCATTAATGCTCACGGCTCCGTGCTGATCAAGGCTAC |
| | | CCATGTCGGCAATGATACCACCCTGGCCCAGATTGTGAAACTGGTG |
| | | GAAGAAGCCCAGATGAGCAAAGCCCCTATTCAGCAGCTGGCTGAC |
| | | CGCTTTTCCGGCTATTTTGTGCCCTTTATCATTATTATGAGCACCCT |
| | | GACCCTGGTGGTGTGGATTGTGATCGGCTTCATTGATTTCGGCGTG |
| | | GTGCAGAGATATTTTCCTAATCCCAACAAACATATTAGCCAGACCG |
| | | AAGTGATTATCCGCTTTGCTTTTCAGACCAGCATTACCGTGCTGTG |
| | | CATTGCTTGCCCCTGCAGCCTGGGCCTGGCTACCCCCACCGCCGTC |
| | | ATGGTCGGCACCGGCGTGGCTGCCCAGAACGGCATTCTGATTAAG |
| | | GGCGGCAAACCTCTGGAAATGGCCCACAAAATCAAAACCGTGATG |
| | | TTTGATAAAACCGGCACCATTACCCACGGCGTGCCCCGCGTGATGC |
| | | GCGTGCTGCTGCTGGGCGATGTGGCTACCCTGCCCCTGCGCAAAGT |
| | | GCTGGCCGTGGTCGGCACCGCCGAAGCTAGCTCCGAACATCCCCT |
| | | GGGCGTGGCCGTGACCAAATATTGCAAAGAAGAACTGGGCACCGA |
| | | AACCCTGGGCTATTGCACCGATTTTCAGGCCGTCCCTGGCTGCGGC |
| | | ATCGGCTGCAAAGTGAGCAATGTGGAGGGCATTCTGGCCCATTCC |
| | | GAAAGACCCCTGTCCGCCCCTGCTAGCCATCTGAATGAAGCCGGC |
| | | AGCCTGCCTGCTGAGAAAGATGCCGTGCCCCAGACCTTTTCCGTGC |
| | | TGATCGGCAACCGCGAATGGCTGCGCCGCAACGGCCTGACCATTA |
| | | GCTCCGATGTCTCCGATGCCATGACCGATCATGAAATGAAAGGCC |
| | | AGACCGCCATTCTGGTGGCCATTGACGGCGTGCTGTGCGGCATGAT |
| | | TGCCATTGCCGATGCCGTGAAACAGGAGGCTGCCCTGGCTGTGCAT |
| | | ACCCTGCAGAGCATGGGCGTGGATGTGGTGCTGATTACCGGCGAT |
| | | AACCGCAAAACCGCTCGCGCCATTGCCACCCAGGTCGGCATTAAC |
| | | AAAGTGTTTGCTGAAGTGCTGCCCAGCCATAAAGTGGCTAAAGTG |
| | | CAGGAACTGCAGAACAAAGGCAAGAAAGTGGCTATGGTCGGCGA |
| | | CGGCGTGAATGATAGCCCTGCCCTGGCCCAGGCCGATATGGGCGT |
| | | GGCTATCGGCACCGGCACCGATGTGGCTATTGAGGCTGCCGATGT |
| | | GGTGCTGATCCGCAATGATCTGCTGGATGTGGTGGCTAGCATTCAT |
| | | CTGAGCAAACGCACCGTCCGCCGCATCCGCATTAATCTGGTGCTGG |
| | | CCCTGATCTATAATCTGGTCGGCATTCCCATCGCCGCCGGCGTGTT |
| | | TATGCCCATCGGCATTGTGCTGCAGCCCTGGATGGGCAGCGCCGCT |
| | | ATGGCCGCTAGCTCCGTGTCCGTGGTGCTGAGCAGCCTGCAGCTGA |
| | | AATGCTATAAGAAACCCGATCTGGAAAGATATGAAGCTCAGGCTC |
| | | ACGGCCATATGAAACCCCTGACCGCCAGCCAGGTGTCCGTGCATA |
| | | TCGGCATGGATGACCGCTGGCGCGATAGCCCCCGCGCCACCCCTTG |
| | | GGATCAGGTGAGCTATGTGAGCCAGGTGTCCCTGAGCTCCCTGACC |
| | | TCCGATAAACCCTCCCGCCATTCCGCCGCTGCCGATGATGACGGCG |
| | | ATAAGTGGAGCCTGCTGCTGAACGGCCGCGATGAAGAACAGTATA |
| | | TCTAAGTCCATAAAGTAGGAAACACTACATCATCCATAAAGTAGG |
| | | AAACACTACATCATCCATAAAGTAGGAAACACTACATCATCCATA |
| | | AAGTAGGAAACACTACAGAATTCGATATCAAGCTTATCGATAATC |
| | | AACCTCTGGATTACAAAATTTGTGAAAGATTGACTGGTATTCTTAA |
| | | CTATGTTGCTCCTTTTACGCTATGTGGATACGCTGCTTTAATGCCTT |
| | | TGTATCATGCTATTGCTTCCCGTATGGCTTTCATTTTCTCCTCCTTGT |
| | | ATAAATCCTGGTTGCTGTCTCTTTATGAGGAGTTGTGGCCCGTTGT |
| | | CAGGCAACGTGGCGTGGTGTGCACTGTGTTTGCTGACGCAACCCCC |
| | | ACTGGTTGGGGCACCTGCAGGAATAAAAGATCTTTATTTTCATTAG |
| | | ATCTGTGTGTTGGTTTTTTGTGTGTCTAGAGTAGATAAGTAGCATG |
| | | GCGGGTTAATCATTAACTACAAGGAACCCCTAGTGATGGAGTTGG |
| | | CCACTCCCTCTCTGCGCGCTCGCTCGCTCACTGAGGCCGGGCGACC |
| | | AAAGGTCGCCCGACGCCCGGGCTTTGCCCGGGCGGCCTCAGTGAG |
| | | CGAGCGAGCGCGCAGAGAGGGAGTGGCCAA |
| 112 | A59 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGGTACCGAGTTAATAATTACCAGCGCGGG |
| | | CCAAATAAATAATCCGCGCCCTAAAATGGGCAAACATTGCAAGCA |
| | | GCAAACAGCAAACACACAGCCCTCCCTGCCTGCTGACCTTGGAGC |
| | | TGGGGCAGAGGTCAGAGACCTCTCTGGGCCCATGCCACCTCCAAC |
| | | ATCCACTCGACCCCTTGGAATTTCGGTGGAGAGGAGCAGAGGTTG |
| | | TCCTGGCGTGGTTTAGGTAGTGTGAGAGGGGAATGACTCCTTTCGG |
| | | TAAGTGCAGTGGAAGCTGTACACTGCCCAGGCAAAGCGTCCGGGC |
| | | AGCGTAGGCGGGCGACTCAGATCCCAGCCAGTGGACTTAGCCCCT |
| | | GTTTGCTCCTCCGATAACTGGGGTGACCTTGGTTAATATTCACCAG |
| | | CAGCCTCCCCCGTTGCCCCTCTGGATCCACTGCTTAAATACGGACG |
| | | AGGACAGGGCCCTGTCTCCTCAGCTTCAGGCACCACCACTGACCTG |
| | | GGACAGTGAATCGAAGAGGTAAGGGTTTAAGGGATGGTTGGTTGG |
| | | TGGGGTATTAATGTTTAATTACCTGGAGCACCTGCCTGAAATCACT |
| | | TTTTTTCAGGTTGGGCGGCCGCCACCATGCCGGAACAAGAACGCC |
| | | AGATCACAGCAAGAGAAGGAGCAAGTAGGAAGATCCTGAGCAAG |
| | | CTCTCCCTGCCCACAAGGGCCTGGGAGCCGGCCATGAAGAAGTCT |
| | | TTTGCCTTCGATAATGTCGGTTATGAAGGCGGGCTCGATGGCCTGG |
| | | GCCCCAGCAGCCAGCCCCAGAAGTGTTTCCTGCAGATCAAGGGCA |
| | | TGACCTGTGCCAGCTGTGTTAGCAATATCGAAAGAAACCTCCAAA |
| | | AAGAGGCTGGAGTTCTGAGTGTTCTAGTGGCTTTAATGGCCGGGA |
| | | AAGCCGAGATCAAGTATGATCCCGAGGTGATTCAGCCATTGGAGA |
| | | TTGCCCAGTTCATCCAGGATCTGGGCTTCGAGGCCGCCGTGATGGA |
| | | GGATTACGCCGGCTCTGATGGCAACATCGAACTGACCATCACCGG |
| | | CATGACCTGTGCTTCCTGCGTGCATAATATCGAGAGCAAACTGACG |
| | | CGGACCAATGGAATCACCTATGCCAGCGTCGCCCTGGCAACCTCTA |
| | | AGGCTCTGGTGAAGTTTGACCCCGAAATCATTGGACCTAGAGACA |
| | | TCATCAAAATTATTGAAGAGATTGGTTTCCACGCCAGCTTAGCTCA |
| | | GCGGAATCCCAACGCACACCATCTTGACCATAAGATGGAAATCAA |
| | | ACAGTGGAAGAAGAGCTTCTTGTGTAGCCTGGTCTTCGGGATACCC |
| | | GTCATGGCACTGATGATTTACATGCTCATTCCGTCCAACGAGCCCC |
| | | ACCAGTCCATGGTGCTGGACCACAACATCATCCCTGGGCTGTCTAT |
| | | CCTCAACCTGATCTTCTTTATTCTGTGCACATTTGTCCAGCTCTTGG |
| | | GAGGCTGGTATTTCTATGTACAGGCCTACAAATCCCTGAGGCATCG |
| | | AAGCGCCAACATGGACGTTTTAATTGTGCTGGCTACATCCATTGCC |
| | | TACGTGTACTCCCTGGTGATCTTAGTGGTTGCTGTGGCTGAAAAGG |
| | | CTGAGAGAAGCCCTGTCACCTTCTTCGACACCCCCCCCATGCTCTT |
| | | CGTGTTTATTGCACTGGGTCGCTGGCTGGAGCATTTAGCCAAGTCC |
| | | AAGACATCCGAGGCTCTGGCCAAGCTGATGTCTCTACAGGCCACG |
| | | GAGGCCACCGTGGTGACTCTGGGTGAAGACAATTTGATAATACGC |
| | | GAGGAACAGGTGCCTATGGAGCTTGTGCAGCGCGGAGATATCGTC |
| | | AAGGTGGTGCCCGGCGGCAAGTTCCCAGTGGATGGAAAAGTGTTA |
| | | GAGGGCAATACCATGGCCGATGAATCCCTCATCACTGGCGAGGCA |
| | | ATGCCGGTGACCAAGAAACCTGGAAGTACTGTCATTGCAGGCAGC |
| | | ATCAATGCCCATGGGAGCGTCCTGATCAAGGCCACCCACGTGGGC |
| | | AATGATACAACTCTCGCCCAGATCGTCAAGCTAGTGGAAGAGGCT |
| | | CAGATGTCTAAAGCTCCAATCCAGCAACTGGCTGACCGCTTCAGCG |
| | | GCTACTTTGTCCCCTTTATCATCATCATGAGCACCCTCACCCTGGTG |
| | | GTGTGGATAGTCATAGGCTTCATTGACTTTGGCGTGGTGCAGAGAT |
| | | ACTTCCCCAACCCTAACAAACATATCTCCCAAACCGAGGTCATTAT |
| | | CAGATTCGCCTTTCAGACATCTATCACTGTGCTGTGCATAGCCTGC |
| | | CCTTGCTCTCTGGGCCTGGCCACCCCCACCGCCGTGATGGTTGGAA |
| | | CCGGGGTCGCTGCACAAAATGGGATCCTGATAAAAGGGGGCAAAC |
| | | CCCTGGAAATGGCACATAAGATTAAAACAGTTATGTTTGATAAGA |
| | | CAGGCACCATTACTCACGGTGTCCCTCGAGTCATGAGAGTGCTGCT |
| | | GCTGGGGGACGTGGCTACACTGCCACTGCGCAAGGTGCTGGCTGT |
| | | GGTGGGCACAGCGGAGGCCTCCTCAGAACACCCATTAGGCGTAGC |
| | | TGTCACAAAATACTGTAAGGAGGAGCTGGGGACAGAGACCCTCGG |
| | | CTATTGTACAGACTTCCAGGCCGTGCCTGGCTGCGGCATCGGCTGT |
| | | AAGGTCAGCAATGTGGAGGGCATCCTGGCACACAGCGAGAGACCC |
| | | CTCTCTGCCCCAGCCTCCCACCTGAATGAAGCTGGGTCTCTGCCTG |
| | | CTGAGAAGGACGCCGTCCCTCAGACCTTCTCCGTGTTGATTGGGAA |
| | | CAGAGAGTGGCTTAGAAGGAATGGCCTGACCATCAGCTCTGACGT |
| | | CTCGGATGCCATGACAGATCACGAGATGAAAGGCCAGACAGCCAT |
| | | ACTGGTTGCCATCGACGGCGTGCTCTGTGGCATGATTGCCATCGCT |
| | | GATGCTGTTAAACAGGAAGCTGCGCTGGCAGTCCACACCCTCCAG |
| | | TCCATGGGAGTGGACGTGGTCCTGATTACTGGAGACAACAGGAAA |
| | | ACCGCTCGGGCCATCGCTACCCAAGTGGGGATAAATAAGGTTTTTG |
| | | CAGAAGTCTTACCCTCCCACAAAGTAGCCAAAGTGCAGGAGCTCC |
| | | AGAACAAGGGTAAAAAGGTGGCAATGGTGGGAGATGGTGTGAAT |
| | | GACAGCCCAGCTCTCGCGCAGGCTGACATGGGCGTAGCCATAGGT |
| | | ACTGGAACAGATGTCGCGATTGAAGCCGCTGACGTAGTCCTCATCC |
| | | GAAACGATTTGCTGGATGTGGTGGCCAGCATCCATCTGTCCAAGCG |
| | | TACCGTGCGCCGGATCCGCATCAACCTGGTCTTGGCCCTCATCTAT |
| | | AATCTCGTGGGCATCCCCATCGCTGCTGGGGTCTTCATGCCCATCG |
| | | GCATTGTGCTGCAGCCCTGGATGGGAAGCGCTGCCATGGCTGCTTC |
| | | TTCTGTCTCCGTTGTGCTGTCCAGCCTACAGCTGAAGTGCTATAAA |
| | | AAGCCAGACCTCGAGCGCTACGAAGCCCAGGCGCATGGCCATATG |
| | | AAGCCCCTCACGGCTTCCCAGGTCTCGGTTCACATTGGGATGGATG |
| | | ACAGATGGCGGGACTCCCCACGCGCCACACCCTGGGACCAGGTAT |
| | | CCTATGTGAGCCAGGTGAGCCTCAGCTCCCTGACATCAGATAAACC |
| | | GTCTAGGCACTCAGCTGCCGCAGATGATGACGGTGACAAATGGAG |
| | | CCTCCTCCTGAACGGCCGCGACGAGGAGCAGTACATCTAAGAATT |
| | | CGATATCAAGCTTATCGATAATCAACCTCTGGATTACAAAATTTGT |
| | | GAAAGATTGACTGGTATTCTTAACTATGTTGCTCCTTTTACGCTATG |
| | | TGGATACGCTGCTTTAATGCCTTTGTATCATGCTATTGCTTCCCGTA |
| | | TGGCTTTCATTTTCTCCTCCTTGTATAAATCCTGGTTGCTGTCTCTTT |
| | | ATGAGGAGTTGTGGCCCGTTGTCAGGCAACGTGGCGTGGTGTGCA |
| | | CTGTGTTTGCTGACGCAACCCCCACTGGTTGGGGCACCTGCAGGAA |
| | | TAAAAGATCTTTATTTTCATTAGATCTGTGTGTTGGTTTTTTGTGTG |
| | | TCTAGAGTAGATAAGTAGCATGGCGGGTTAATCATTAACTACAAG |
| | | GAACCCCTAGTGATGGAGTTGGCCACTCCCTCTCTGCGCGCTCGCT |
| | | CGCTCACTGAGGCCGGGCGACCAAAGGTCGCCCGACGCCCGGGCT |
| | | TTGCCCGGGCGGCCTCAGTGAGCGAGCGAGCGCGCAGAGAGGGAG |
| | | TGGCCAA |
| 113 | A60 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGGTACCGAGTTAATAATTACCAGCGCGGG |
| | | CCAAATAAATAATCCGCGCCCTAAAATGGGCAAACATTGCAAGCA |
| | | GCAAACAGCAAACACACAGCCCTCCCTGCCTGCTGACCTTGGAGC |
| | | TGGGGCAGAGGTCAGAGACCTCTCTGGGCCCATGCCACCTCCAAC |
| | | ATCCACTCGACCCCTTGGAATTTCGGTGGAGAGGAGCAGAGGTTG |
| | | TCCTGGCGTGGTTTAGGTAGTGTGAGAGGGGAATGACTCCTTTCGG |
| | | TAAGTGCAGTGGAAGCTGTACACTGCCCAGGCAAAGCGTCCGGGC |
| | | AGCGTAGGCGGGCGACTCAGATCCCAGCCAGTGGACTTAGCCCCT |
| | | GTTTGCTCCTCCGATAACTGGGGTGACCTTGGTTAATATTCACCAG |
| | | CAGCCTCCCCCGTTGCCCCTCTGGATCCACTGCTTAAATACGGACG |
| | | AGGACAGGGCCCTGTCTCCTCAGCTTCAGGCACCACCACTGACCTG |
| | | GGACAGTGAATCGAAGAGGTAAGGGTTTAAGGGATGGTTGGTTGG |
| | | TGGGGTATTAATGTTTAATTACCTGGAGCACCTGCCTGAAATCACT |
| | | TTTTTTCAGGTTGGGCGGCCGCCACCATGCCCGAACAGGAAAGAC |
| | | AGATTACCGCCCGCGAGGGCGCTTCCCGCAAAATTCTGAGCAAAC |
| | | TGAGCCTGCCCACCCGCGCCTGGGAACCTGCCATGAAGAAAAGCT |
| | | TTGCTTTTGATAATGTCGGCTATGAGGGCGGCCTGGACGGCCTGGG |
| | | CCCTAGCAGCCAGGTGGCTACCACCCTGATTGCCATTGCCGGCATG |
| | | ACCTGCGCCAGCTGCGTGCATAGCATTGAGGGCATGATTAGCCAG |
| | | CTGGAGGGCGTGCAGCAGATTTCCGTGAGCCTGGCTGAGGGCACC |
| | | GCTACCGTGCTGTATAATCCCTCCGTGATTAGCCCCGAAGAACTGC |
| | | GCGCTGCCATTGAAGATATGGGCTTTGAAGCCTCCGTGGTGTCCGA |
| | | AAGCTGCAGCACCAATCCCCTGGGCAATCATTCCGCCGGCAATAG |
| | | CATGGTGCAGACCACCGACGGCACCCCCACCTCCGTGCAGGAAGT |
| | | GGCTCCCCATACCGGCCGCCTGCCCGCCAATCATGCCCCTGATATT |
| | | CTGGCTAAAAGCCCCCAGAGCACCCGCGCCGTGGCTCCTCAGAAA |
| | | TGCTTTCTGCAGATTAAGGGCATGACCTGCGCTAGCTGCGTGAGCA |
| | | ATATTGAACGCAATCTGCAGAAAGAAGCCGGCGTGCTGTCCGTGC |
| | | TGGTGGCCCTGATGGCCGGCAAAGCTGAAATCAAATATGATCCCG |
| | | AAGTGATTCAGCCCCTGGAAATTGCCCAGTTCATTCAGGATCTGGG |
| | | CTTTGAGGCTGCCGTGATGGAAGATTATGCCGGCTCCGACGGCAAT |
| | | ATTGAACTGACCATTACCGGCATGACCTGCGCCAGCTGCGTGCATA |
| | | ATATTGAAAGCAAACTGACCCGCACCAACGGCATTACCTATGCTTC |
| | | CGTGGCCCTGGCTACCAGCAAAGCCCTGGTGAAATTTGATCCTGAA |
| | | ATTATCGGCCCCCGCGATATTATCAAAATTATTGAAGAAATCGGCT |
| | | TTCACGCCAGCCTGGCTCAGCGCAATCCCAATGCCCATCATCTGGA |
| | | TCATAAGATGGAAATCAAACAGTGGAAGAAAAGCTTTCTGTGCAG |
| | | CCTGGTGTTCGGCATTCCTGTGATGGCCCTGATGATCTATATGCTG |
| | | ATTCCCAGCAATGAACCCCATCAGAGCATGGTGCTGGATCATAAT |
| | | ATTATTCCCGGCCTGAGCATTCTGAATCTGATTTTCTTTATCCTGTG |
| | | CACCTTTGTGCAGCTGCTGGGCGGCTGGTATTTCTATGTGCAGGCT |
| | | TATAAAAGCCTGCGCCACCGCTCCGCCAATATGGATGTGCTGATTG |
| | | TGCTGGCCACCAGCATTGCTTATGTCTATAGCCTGGTCATTCTGGT |
| | | GGTGGCCGTGGCCGAGAAAGCTGAACGCAGCCCCGTGACCTTCTT |
| | | TGATACCCCTCCCATGCTGTTCGTGTTTATCGCTCTGGGCCGCTGGC |
| | | TGGAACATCTGGCCAAAAGCAAAACCTCCGAAGCCCTGGCTAAAC |
| | | TGATGAGCCTGCAGGCCACCGAAGCCACCGTGGTGACCCTGGGCG |
| | | AAGATAATCTGATTATCCGCGAAGAACAGGTGCCTATGGAACTGG |
| | | TGCAGCGCGGCGATATTGTGAAAGTGGTGCCCGGCGGCAAATTTC |
| | | CCGTGGACGGCAAAGTGCTGGAGGGCAATACCATGGCTGATGAAT |
| | | CCCTGATTACCGGCGAAGCTATGCCTGTGACCAAGAAACCCGGCA |
| | | GCACCGTGATTGCCGGCAGCATTAATGCTCACGGCTCCGTGCTGAT |
| | | CAAGGCTACCCATGTCGGCAATGATACCACCCTGGCCCAGATTGTG |
| | | AAACTGGTGGAAGAAGCCCAGATGAGCAAAGCCCCTATTCAGCAG |
| | | CTGGCTGACCGCTTTTCCGGCTATTTTGTGCCCTTTATCATTATTAT |
| | | GAGCACCCTGACCCTGGTGGTGTGGATTGTGATCGGCTTCATTGAT |
| | | TTCGGCGTGGTGCAGAGATATTTTCCTAATCCCAACAAACATATTA |
| | | GCCAGACCGAAGTGATTATCCGCTTTGCTTTTCAGACCAGCATTAC |
| | | CGTGCTGTGCATTGCTTGCCCCTGCAGCCTGGGCCTGGCTACCCCC |
| | | ACCGCCGTCATGGTCGGCACCGGCGTGGCTGCCCAGAACGGCATT |
| | | CTGATTAAGGGCGGCAAACCTCTGGAAATGGCCCACAAAATCAAA |
| | | ACCGTGATGTTTGATAAAACCGGCACCATTACCCACGGCGTGCCCC |
| | | GCGTGATGCGCGTGCTGCTGCTGGGCGATGTGGCTACCCTGCCCCT |
| | | GCGCAAAGTGCTGGCCGTGGTCGGCACCGCCGAAGCTAGCTCCGA |
| | | ACATCCCCTGGGCGTGGCCGTGACCAAATATTGCAAAGAAGAACT |
| | | GGGCACCGAAACCCTGGGCTATTGCACCGATTTTCAGGCCGTCCCT |
| | | GGCTGCGGCATCGGCTGCAAAGTGAGCAATGTGGAGGGCATTCTG |
| | | GCCCATTCCGAAAGACCCCTGTCCGCCCCTGCTAGCCATCTGAATG |
| | | AAGCCGGCAGCCTGCCTGCTGAGAAAGATGCCGTGCCCCAGACCT |
| | | TTTCCGTGCTGATCGGCAACCGCGAATGGCTGCGCCGCAACGGCCT |
| | | GACCATTAGCTCCGATGTCTCCGATGCCATGACCGATCATGAAATG |
| | | AAAGGCCAGACCGCCATTCTGGTGGCCATTGACGGCGTGCTGTGC |
| | | GGCATGATTGCCATTGCCGATGCCGTGAAACAGGAGGCTGCCCTG |
| | | GCTGTGCATACCCTGCAGAGCATGGGCGTGGATGTGGTGCTGATTA |
| | | CCGGCGATAACCGCAAAACCGCTCGCGCCATTGCCACCCAGGTCG |
| | | GCATTAACAAAGTGTTTGCTGAAGTGCTGCCCAGCCATAAAGTGG |
| | | CTAAAGTGCAGGAACTGCAGAACAAAGGCAAGAAAGTGGCTATG |
| | | GTCGGCGACGGCGTGAATGATAGCCCTGCCCTGGCCCAGGCCGAT |
| | | ATGGGCGTGGCTATCGGCACCGGCACCGATGTGGCTATTGAGGCT |
| | | GCCGATGTGGTGCTGATCCGCAATGATCTGCTGGATGTGGTGGCTA |
| | | GCATTCATCTGAGCAAACGCACCGTCCGCCGCATCCGCATTAATCT |
| | | GGTGCTGGCCCTGATCTATAATCTGGTCGGCATTCCCATCGCCGCC |
| | | GGCGTGTTTATGCCCATCGGCATTGTGCTGCAGCCCTGGATGGGCA |
| | | GCGCCGCTATGGCCGCTAGCTCCGTGTCCGTGGTGCTGAGCAGCCT |
| | | GCAGCTGAAATGCTATAAGAAACCCGATCTGGAAAGATATGAAGC |
| | | TCAGGCTCACGGCCATATGAAACCCCTGACCGCCAGCCAGGTGTC |
| | | CGTGCATATCGGCATGGATGACCGCTGGCGCGATAGCCCCCGCGC |
| | | CACCCCTTGGGATCAGGTGAGCTATGTGAGCCAGGTGTCCCTGAGC |
| | | TCCCTGACCTCCGATAAACCCTCCCGCCATTCCGCCGCTGCCGATG |
| | | ATGACGGCGATAAGTGGAGCCTGCTGCTGAACGGCCGCGATGAAG |
| | | AACAGTATATCTAAGTCCATAAAGTAGGAAACACTACATCATCCA |
| | | TAAAGTAGGAAACACTACATCATCCATAAAGTAGGAAACACTACA |
| | | TCATCCATAAAGTAGGAAACACTACAGAATTCGATATCAAGCTTAT |
| | | CGATAATCAACCTCTGGATTACAAAATTTGTGAAAGATTGACTGGT |
| | | ATTCTTAACTATGTTGCTCCTTTTACGCTATGTGGATACGCTGCTTT |
| | | AATGCCTTTGTATCATGCTATTGCTTCCCGTATGGCTTTCATTTTCT |
| | | CCTCCTTGTATAAATCCTGGTTGCTGTCTCTTTATGAGGAGTTGTGG |
| | | CCCGTTGTCAGGCAACGTGGCGTGGTGTGCACTGTGTTTGCTGACG |
| | | CAACCCCCACTGGTTGGGGCACCTGCAGGAATAAAAGATCTTTATT |
| | | TTCATTAGATCTGTGTGTTGGTTTTTTGTGTGTCTAGAGTAGATAAG |
| | | TAGCATGGCGGGTTAATCATTAACTACAAGGAACCCCTAGTGATG |
| | | GAGTTGGCCACTCCCTCTCTGCGCGCTCGCTCGCTCACTGAGGCCG |
| | | GGCGACCAAAGGTCGCCCGACGCCCGGGCTTTGCCCGGGCGGCCT |
| | | CAGTGAGCGAGCGAGCGCGCAGAGAGGGAGTGGCCAA |
| 114 | A61 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGGTACCGAGTTAATAATTACCAGCGCGGG |
| | | CCAAATAAATAATCCGCGAGGGGCAGCTGGACTTTGGACTCCGCG |
| | | CGCTGGTAATTATTAACCTCGCGAATATTGATTCGAGGCCGCGATT |
| | | GCCGCAATCGCGAGGGGCAGGTGACCTTTGCCCAGCGCGCGTTCG |
| | | CCCCGCCCCGGACCCCTAAAATGGGCAAACATTGCAAGCAGCAAA |
| | | CAGCAAACACACAGCCCTCCCTGCCTGCTGACCTTGGAGCTGGGG |
| | | CAGAGGTCAGAGACCTCTCTGGGCCCATGCCACCTCCAACATCCAC |
| | | TCGACCCCTTGGAATTTCGGTGGAGAGGAGCAGAGGTTGTCCTGG |
| | | CGTGGTTTAGGTAGTGTGAGAGGGGAATGACTCCTTTCGGTAAGTG |
| | | CAGTGGAAGCTGTACACTGCCCAGGCAAAGCGTCCGGGCAGCGTA |
| | | GGCGGGCGACTCAGATCCCAGCCAGTGGACTTAGCCCCTGTTTGCT |
| | | CCTCCGATAACTGGGGTGACCTTGGTTAATATTCACCAGCAGCCTC |
| | | CCCCGTTGCCCCTCTGGATCCACTGCTTAAATACGGACGAGGACAG |
| | | GGCCCTGTCTCCTCAGCTTCAGGCACCACCACTGACCTGGGACAGT |
| | | GAATCGAAGAGGTAAGGGTTTAAGGGATGGTTGGTTGGTGGGGTA |
| | | TTAATGTTTAATTACCTGGAGCACCTGCCTGAAATCACTTTTTTTCA |
| | | GGTTGGGCGGCCGCCACCATGCCGGAACAAGAACGCCAGATCACA |
| | | GCAAGAGAAGGAGCAAGTAGGAAGATCCTGAGCAAGCTCTCCCTG |
| | | CCCACAAGGGCCTGGGAGCCGGCCATGAAGAAGTCTTTTGCCTTC |
| | | GATAATGTCGGTTATGAAGGCGGGCTCGATGGCCTGGGCCCCAGC |
| | | AGCCAGCCCCAGAAGTGTTTCCTGCAGATCAAGGGCATGACCTGT |
| | | GCCAGCTGTGTTAGCAATATCGAAAGAAACCTCCAAAAAGAGGCT |
| | | GGAGTTCTGAGTGTTCTAGTGGCTTTAATGGCCGGGAAAGCCGAG |
| | | ATCAAGTATGATCCCGAGGTGATTCAGCCATTGGAGATTGCCCAGT |
| | | TCATCCAGGATCTGGGCTTCGAGGCCGCCGTGATGGAGGATTACG |
| | | CCGGCTCTGATGGCAACATCGAACTGACCATCACCGGCATGACCT |
| | | GTGCTTCCTGCGTGCATAATATCGAGAGCAAACTGACGCGGACCA |
| | | ATGGAATCACCTATGCCAGCGTCGCCCTGGCAACCTCTAAGGCTCT |
| | | GGTGAAGTTTGACCCCGAAATCATTGGACCTAGAGACATCATCAA |
| | | AATTATTGAAGAGATTGGTTTCCACGCCAGCTTAGCTCAGCGGAAT |
| | | CCCAACGCACACCATCTTGACCATAAGATGGAAATCAAACAGTGG |
| | | AAGAAGAGCTTCTTGTGTAGCCTGGTCTTCGGGATACCCGTCATGG |
| | | CACTGATGATTTACATGCTCATTCCGTCCAACGAGCCCCACCAGTC |
| | | CATGGTGCTGGACCACAACATCATCCCTGGGCTGTCTATCCTCAAC |
| | | CTGATCTTCTTTATTCTGTGCACATTTGTCCAGCTCTTGGGAGGCTG |
| | | GTATTTCTATGTACAGGCCTACAAATCCCTGAGGCATCGAAGCGCC |
| | | AACATGGACGTTTTAATTGTGCTGGCTACATCCATTGCCTACGTGT |
| | | ACTCCCTGGTGATCTTAGTGGTTGCTGTGGCTGAAAAGGCTGAGAG |
| | | AAGCCCTGTCACCTTCTTCGACACCCCCCCCATGCTCTTCGTGTTTA |
| | | TTGCACTGGGTCGCTGGCTGGAGCATTTAGCCAAGTCCAAGACATC |
| | | CGAGGCTCTGGCCAAGCTGATGTCTCTACAGGCCACGGAGGCCAC |
| | | CGTGGTGACTCTGGGTGAAGACAATTTGATAATACGCGAGGAACA |
| | | GGTGCCTATGGAGCTTGTGCAGCGCGGAGATATCGTCAAGGTGGT |
| | | GCCCGGCGGCAAGTTCCCAGTGGATGGAAAAGTGTTAGAGGGCAA |
| | | TACCATGGCCGATGAATCCCTCATCACTGGCGAGGCAATGCCGGT |
| | | GACCAAGAAACCTGGAAGTACTGTCATTGCAGGCAGCATCAATGC |
| | | CCATGGGAGCGTCCTGATCAAGGCCACCCACGTGGGCAATGATAC |
| | | AACTCTCGCCCAGATCGTCAAGCTAGTGGAAGAGGCTCAGATGTC |
| | | TAAAGCTCCAATCCAGCAACTGGCTGACCGCTTCAGCGGCTACTTT |
| | | GTCCCCTTTATCATCATCATGAGCACCCTCACCCTGGTGGTGTGGA |
| | | TAGTCATAGGCTTCATTGACTTTGGCGTGGTGCAGAGATACTTCCC |
| | | CAACCCTAACAAACATATCTCCCAAACCGAGGTCATTATCAGATTC |
| | | GCCTTTCAGACATCTATCACTGTGCTGTGCATAGCCTGCCCTTGCTC |
| | | TCTGGGCCTGGCCACCCCCACCGCCGTGATGGTTGGAACCGGGGTC |
| | | GCTGCACAAAATGGGATCCTGATAAAAGGGGGCAAACCCCTGGAA |
| | | ATGGCACATAAGATTAAAACAGTTATGTTTGATAAGACAGGCACC |
| | | ATTACTCACGGTGTCCCTCGAGTCATGAGAGTGCTGCTGCTGGGGG |
| | | ACGTGGCTACACTGCCACTGCGCAAGGTGCTGGCTGTGGTGGGCA |
| | | CAGCGGAGGCCTCCTCAGAACACCCATTAGGCGTAGCTGTCACAA |
| | | AATACTGTAAGGAGGAGCTGGGGACAGAGACCCTCGGCTATTGTA |
| | | CAGACTTCCAGGCCGTGCCTGGCTGCGGCATCGGCTGTAAGGTCA |
| | | GCAATGTGGAGGGCATCCTGGCACACAGCGAGAGACCCCTCTCTG |
| | | CCCCAGCCTCCCACCTGAATGAAGCTGGGTCTCTGCCTGCTGAGAA |
| | | GGACGCCGTCCCTCAGACCTTCTCCGTGTTGATTGGGAACAGAGAG |
| | | TGGCTTAGAAGGAATGGCCTGACCATCAGCTCTGACGTCTCGGATG |
| | | CCATGACAGATCACGAGATGAAAGGCCAGACAGCCATACTGGTTG |
| | | CCATCGACGGCGTGCTCTGTGGCATGATTGCCATCGCTGATGCTGT |
| | | TAAACAGGAAGCTGCGCTGGCAGTCCACACCCTCCAGTCCATGGG |
| | | AGTGGACGTGGTCCTGATTACTGGAGACAACAGGAAAACCGCTCG |
| | | GGCCATCGCTACCCAAGTGGGGATAAATAAGGTTTTTGCAGAAGT |
| | | CTTACCCTCCCACAAAGTAGCCAAAGTGCAGGAGCTCCAGAACAA |
| | | GGGTAAAAAGGTGGCAATGGTGGGAGATGGTGTGAATGACAGCCC |
| | | AGCTCTCGCGCAGGCTGACATGGGCGTAGCCATAGGTACTGGAAC |
| | | AGATGTCGCGATTGAAGCCGCTGACGTAGTCCTCATCCGAAACGA |
| | | TTTGCTGGATGTGGTGGCCAGCATCCATCTGTCCAAGCGTACCGTG |
| | | CGCCGGATCCGCATCAACCTGGTCTTGGCCCTCATCTATAATCTCG |
| | | TGGGCATCCCCATCGCTGCTGGGGTCTTCATGCCCATCGGCATTGT |
| | | GCTGCAGCCCTGGATGGGAAGCGCTGCCATGGCTGCTTCTTCTGTC |
| | | TCCGTTGTGCTGTCCAGCCTACAGCTGAAGTGCTATAAAAAGCCAG |
| | | ACCTCGAGCGCTACGAAGCCCAGGCGCATGGCCATATGAAGCCCC |
| | | TCACGGCTTCCCAGGTCTCGGTTCACATTGGGATGGATGACAGATG |
| | | GCGGGACTCCCCACGCGCCACACCCTGGGACCAGGTATCCTATGT |
| | | GAGCCAGGTGAGCCTCAGCTCCCTGACATCAGATAAACCGTCTAG |
| | | GCACTCAGCTGCCGCAGATGATGACGGTGACAAATGGAGCCTCCT |
| | | CCTGAACGGCCGCGACGAGGAGCAGTACATCTAAGAATTCGATAT |
| | | CAAGCTTATCGATAATCAACCTCTGGATTACAAAATTTGTGAAAGA |
| | | TTGACTGGTATTCTTAACTATGTTGCTCCTTTTACGCTATGTGGATA |
| | | CGCTGCTTTAATGCCTTTGTATCATGCTATTGCTTCCCGTATGGCTT |
| | | TCATTTTCTCCTCCTTGTATAAATCCTGGTTGCTGTCTCTTTATGAG |
| | | GAGTTGTGGCCCGTTGTCAGGCAACGTGGCGTGGTGTGCACTGTGT |
| | | TTGCTGACGCAACCCCCACTGGTTGGGGCACCTGCAGGAATAAAA |
| | | GATCTTTATTTTCATTAGATCTGTGTGTTGGTTTTTTGTGTGTCTAG |
| | | AGTAGATAAGTAGCATGGGGGTTAATCATTAACTACAAGGAACC |
| | | CCTAGTGATGGAGTTGGCCACTCCCTCTCTGCGCGCTCGCTCGCTC |
| | | ACTGAGGCCGGGCGACCAAAGGTCGCCCGACGCCCGGGCTTTGCC |
| | | CGGGCGGCCTCAGTGAGCGAGCGAGCGCGCAGAGAGGGAGTGGC |
| | | CAA |
| 115 | A62 | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGAGAGGGAGTGGCCAACTCCATCACTA |
| | | GGGGTTCCTGGAGGGGTGGAGTCGTGACGTGAATTACGTCATAGG |
| | | GTTAGGGAGGTCTAGAGGTACCGAGTTAATAATTACCAGCGCGGG |
| | | CCAAATAAATAATCCGCGAGGGGCAGCTGGACTTTGGACTCCGCG |
| | | CGCTGGTAATTATTAACCTCGCGAATATTGATTCGAGGCCGCGATT |
| | | GCCGCAATCGCGAGGGGCAGGTGACCTTTGCCCAGCGCGCGTTCG |
| | | CCCCGCCCCGGACCCCTAAAATGGGCAAACATTGCAAGCAGCAAA |
| | | CAGCAAACACACAGCCCTCCCTGCCTGCTGACCTTGGAGCTGGGG |
| | | CAGAGGTCAGAGACCTCTCTGGGCCCATGCCACCTCCAACATCCAC |
| | | TCGACCCCTTGGAATTTCGGTGGAGAGGAGCAGAGGTTGTCCTGG |
| | | CGTGGTTTAGGTAGTGTGAGAGGGGAATGACTCCTTTCGGTAAGTG |
| | | CAGTGGAAGCTGTACACTGCCCAGGCAAAGCGTCCGGGCAGCGTA |
| | | GGCGGGCGACTCAGATCCCAGCCAGTGGACTTAGCCCCTGTTTGCT |
| | | CCTCCGATAACTGGGGTGACCTTGGTTAATATTCACCAGCAGCCTC |
| | | CCCCGTTGCCCCTCTGGATCCACTGCTTAAATACGGACGAGGACAG |
| | | GGCCCTGTCTCCTCAGCTTCAGGCACCACCACTGACCTGGGACAGT |
| | | GAATCGAAGAGGTAAGGGTTTAAGGGATGGTTGGTTGGTGGGGTA |
| | | TTAATGTTTAATTACCTGGAGCACCTGCCTGAAATCACTTTTTTTCA |
| | | GGTTGGGCGGCCGCCACCATGCCCGAACAGGAAAGACAGATTACC |
| | | GCCCGCGAGGGCGCTTCCCGCAAAATTCTGAGCAAACTGAGCCTG |
| | | CCCACCCGCGCCTGGGAACCTGCCATGAAGAAAAGCTTTGCTTTTG |
| | | ATAATGTCGGCTATGAGGGCGGCCTGGACGGCCTGGGCCCTAGCA |
| | | GCCAGGTGGCTACCACCCTGATTGCCATTGCCGGCATGACCTGCGC |
| | | CAGCTGCGTGCATAGCATTGAGGGCATGATTAGCCAGCTGGAGGG |
| | | CGTGCAGCAGATTTCCGTGAGCCTGGCTGAGGGCACCGCTACCGT |
| | | GCTGTATAATCCCTCCGTGATTAGCCCCGAAGAACTGCGCGCTGCC |
| | | ATTGAAGATATGGGCTTTGAAGCCTCCGTGGTGTCCGAAAGCTGCA |
| | | GCACCAATCCCCTGGGCAATCATTCCGCCGGCAATAGCATGGTGC |
| | | AGACCACCGACGGCACCCCCACCTCCGTGCAGGAAGTGGCTCCCC |
| | | ATACCGGCCGCCTGCCCGCCAATCATGCCCCTGATATTCTGGCTAA |
| | | AAGCCCCCAGAGCACCCGCGCCGTGGCTCCTCAGAAATGCTTTCTG |
| | | CAGATTAAGGGCATGACCTGCGCTAGCTGCGTGAGCAATATTGAA |
| | | CGCAATCTGCAGAAAGAAGCCGGCGTGCTGTCCGTGCTGGTGGCC |
| | | CTGATGGCCGGCAAAGCTGAAATCAAATATGATCCCGAAGTGATT |
| | | CAGCCCCTGGAAATTGCCCAGTTCATTCAGGATCTGGGCTTTGAGG |
| | | CTGCCGTGATGGAAGATTATGCCGGCTCCGACGGCAATATTGAACT |
| | | GACCATTACCGGCATGACCTGCGCCAGCTGCGTGCATAATATTGAA |
| | | AGCAAACTGACCCGCACCAACGGCATTACCTATGCTTCCGTGGCCC |
| | | TGGCTACCAGCAAAGCCCTGGTGAAATTTGATCCTGAAATTATCGG |
| | | CCCCCGCGATATTATCAAAATTATTGAAGAAATCGGCTTTCACGCC |
| | | AGCCTGGCTCAGCGCAATCCCAATGCCCATCATCTGGATCATAAGA |
| | | TGGAAATCAAACAGTGGAAGAAAAGCTTTCTGTGCAGCCTGGTGT |
| | | TCGGCATTCCTGTGATGGCCCTGATGATCTATATGCTGATTCCCAG |
| | | CAATGAACCCCATCAGAGCATGGTGCTGGATCATAATATTATTCCC |
| | | GGCCTGAGCATTCTGAATCTGATTTTCTTTATCCTGTGCACCTTTGT |
| | | GCAGCTGCTGGGCGGCTGGTATTTCTATGTGCAGGCTTATAAAAGC |
| | | CTGCGCCACCGCTCCGCCAATATGGATGTGCTGATTGTGCTGGCCA |
| | | CCAGCATTGCTTATGTCTATAGCCTGGTCATTCTGGTGGTGGCCGT |
| | | GGCCGAGAAAGCTGAACGCAGCCCCGTGACCTTCTTTGATACCCCT |
| | | CCCATGCTGTTCGTGTTTATCGCTCTGGGCCGCTGGCTGGAACATC |
| | | TGGCCAAAAGCAAAACCTCCGAAGCCCTGGCTAAACTGATGAGCC |
| | | TGCAGGCCACCGAAGCCACCGTGGTGACCCTGGGCGAAGATAATC |
| | | TGATTATCCGCGAAGAACAGGTGCCTATGGAACTGGTGCAGCGCG |
| | | GCGATATTGTGAAAGTGGTGCCCGGCGGCAAATTTCCCGTGGACG |
| | | GCAAAGTGCTGGAGGGCAATACCATGGCTGATGAATCCCTGATTA |
| | | CCGGCGAAGCTATGCCTGTGACCAAGAAACCCGGCAGCACCGTGA |
| | | TTGCCGGCAGCATTAATGCTCACGGCTCCGTGCTGATCAAGGCTAC |
| | | CCATGTCGGCAATGATACCACCCTGGCCCAGATTGTGAAACTGGTG |
| | | GAAGAAGCCCAGATGAGCAAAGCCCCTATTCAGCAGCTGGCTGAC |
| | | CGCTTTTCCGGCTATTTTGTGCCCTTTATCATTATTATGAGCACCCT |
| | | GACCCTGGTGGTGTGGATTGTGATCGGCTTCATTGATTTCGGCGTG |
| | | GTGCAGAGATATTTTCCTAATCCCAACAAACATATTAGCCAGACCG |
| | | AAGTGATTATCCGCTTTGCTTTTCAGACCAGCATTACCGTGCTGTG |
| | | CATTGCTTGCCCCTGCAGCCTGGGCCTGGCTACCCCCACCGCCGTC |
| | | ATGGTCGGCACCGGCGTGGCTGCCCAGAACGGCATTCTGATTAAG |
| | | GGCGGCAAACCTCTGGAAATGGCCCACAAAATCAAAACCGTGATG |
| | | TTTGATAAAACCGGCACCATTACCCACGGCGTGCCCCGCGTGATGC |
| | | GCGTGCTGCTGCTGGGCGATGTGGCTACCCTGCCCCTGCGCAAAGT |
| | | GCTGGCCGTGGTCGGCACCGCCGAAGCTAGCTCCGAACATCCCCT |
| | | GGGCGTGGCCGTGACCAAATATTGCAAAGAAGAACTGGGCACCGA |
| | | AACCCTGGGCTATTGCACCGATTTTCAGGCCGTCCCTGGCTGCGGC |
| | | ATCGGCTGCAAAGTGAGCAATGTGGAGGGCATTCTGGCCCATTCC |
| | | GAAAGACCCCTGTCCGCCCCTGCTAGCCATCTGAATGAAGCCGGC |
| | | AGCCTGCCTGCTGAGAAAGATGCCGTGCCCCAGACCTTTTCCGTGC |
| | | TGATCGGCAACCGCGAATGGCTGCGCCGCAACGGCCTGACCATTA |
| | | GCTCCGATGTCTCCGATGCCATGACCGATCATGAAATGAAAGGCC |
| | | AGACCGCCATTCTGGTGGCCATTGACGGCGTGCTGTGCGGCATGAT |
| | | TGCCATTGCCGATGCCGTGAAACAGGAGGCTGCCCTGGCTGTGCAT |
| | | ACCCTGCAGAGCATGGGCGTGGATGTGGTGCTGATTACCGGCGAT |
| | | AACCGCAAAACCGCTCGCGCCATTGCCACCCAGGTCGGCATTAAC |
| | | AAAGTGTTTGCTGAAGTGCTGCCCAGCCATAAAGTGGCTAAAGTG |
| | | CAGGAACTGCAGAACAAAGGCAAGAAAGTGGCTATGGTCGGCGA |
| | | CGGCGTGAATGATAGCCCTGCCCTGGCCCAGGCCGATATGGGCGT |
| | | GGCTATCGGCACCGGCACCGATGTGGCTATTGAGGCTGCCGATGT |
| | | GGTGCTGATCCGCAATGATCTGCTGGATGTGGTGGCTAGCATTCAT |
| | | CTGAGCAAACGCACCGTCCGCCGCATCCGCATTAATCTGGTGCTGG |
| | | CCCTGATCTATAATCTGGTCGGCATTCCCATCGCCGCCGGCGTGTT |
| | | TATGCCCATCGGCATTGTGCTGCAGCCCTGGATGGGCAGCGCCGCT |
| | | ATGGCCGCTAGCTCCGTGTCCGTGGTGCTGAGCAGCCTGCAGCTGA |
| | | AATGCTATAAGAAACCCGATCTGGAAAGATATGAAGCTCAGGCTC |
| | | ACGGCCATATGAAACCCCTGACCGCCAGCCAGGTGTCCGTGCATA |
| | | TCGGCATGGATGACCGCTGGCGCGATAGCCCCCGCGCCACCCCTTG |
| | | GGATCAGGTGAGCTATGTGAGCCAGGTGTCCCTGAGCTCCCTGACC |
| | | TCCGATAAACCCTCCCGCCATTCCGCCGCTGCCGATGATGACGGCG |
| | | ATAAGTGGAGCCTGCTGCTGAACGGCCGCGATGAAGAACAGTATA |
| | | TCTAAGTCCATAAAGTAGGAAACACTACATCATCCATAAAGTAGG |
| | | AAACACTACATCATCCATAAAGTAGGAAACACTACATCATCCATA |
| | | AAGTAGGAAACACTACAGAATTCGATATCAAGCTTATCGATAATC |
| | | AACCTCTGGATTACAAAATTTGTGAAAGATTGACTGGTATTCTTAA |
| | | CTATGTTGCTCCTTTTACGCTATGTGGATACGCTGCTTTAATGCCTT |
| | | TGTATCATGCTATTGCTTCCCGTATGGCTTTCATTTTCTCCTCCTTGT |
| | | ATAAATCCTGGTTGCTGTCTCTTTATGAGGAGTTGTGGCCCGTTGT |
| | | CAGGCAACGTGGCGTGGTGTGCACTGTGTTTGCTGACGCAACCCCC |
| | | ACTGGTTGGGGCACCTGCAGGAATAAAAGATCTTTATTTTCATTAG |
| | | ATCTGTGTGTTGGTTTTTTGTGTGTCTAGAGTAGATAAGTAGCATG |
| | | GCGGGTTAATCATTAACTACAAGGAACCCCTAGTGATGGAGTTGG |
| | | CCACTCCCTCTCTGCGCGCTCGCTCGCTCACTGAGGCCGGGCGACC |
| | | AAAGGTCGCCCGACGCCCGGGCTTTGCCCGGGCGGCCTCAGTGAG |
| | | CGAGCGAGCGCGCAGAGAGGGAGTGGCCAA |

TABLE 12
Protein sequences

| Transgene | | | |
|---|---|---|---|
| | SEQ | | SEQ |
| | ID | Corresponding protein sequence | ID |
| Name | NO | Sequence | NO |
| miniA | 41 | MPEQERQITAREGASRKILSKLSLPTRAWEPAMKKSFAFDNVGYEGG | 118 |
| TP7B | | LDGLGPSSQPQKCFLQIKGMTCASCVSNIERNLQKEAGVLSVLVALM | |
| v.co | | AGKAEIKYDPEVIQPLEIAQFIQDLGFEAAVMEDYAGSDGNIELTIT | |
| | | GMTCASCVHNIESKLTRTNGITYASVALATSKALVKFDPEIIGPRDI | |
| | | IKIIEEIGFHASLAQRNPNAHHLDHKMEIKQWKKSFLCSLVFGIPVM | |
| | | ALMIYMLIPSNEPHQSMVLDHNIIPGLSILNLIFFILCTFVQLLGGW | |
| | | YFYVQAYKSLRHRSANMDVLIVLATSIAYVYSLVILVVAVAEKAERS | |
| | | PVTFFDTPPMLFVFIALGRWLEHLAKSKTSEALAKLMSLQATEATVV | |
| | | TLGEDNLIIREEQVPMELVQRGDIVKVVPGGKFPVDGKVLEGNTMAD | |
| | | ESLITGEAMPVTKKPGSTVIAGSINAHGSVLIKATHVGNDTTLAQIV | |
| | | KLVEEAQMSKAPIQQLADRFSGYFVPFIIIMSTLTLVVWIVIGFIDF | |
| | | GVVQRYFPNPNKHISQTEVIIRFAFQTSITVLCIACPCSLGLATPTA | |
| | | VMVGTGVAAQNGILIKGGKPLEMAHKIKTVMFDKTGTITHGVPRVMR | |
| | | VLLLGDVATLPLRKVLAVVGTAEASSEHPLGVAVTKYCKEELGTETL | |
| | | GYCTDFQAVPGCGIGCKVSNVEGILAHSERPLSAPASHLNEAGSLPA | |
| | | EKDAVPQTFSVLIGNREWLRRNGLTISSDVSDAMTDHEMKGQTAILV | |
| | | AIDGVLCGMIAIADAVKQEAALAVHTLQSMGVDVVLITGDNRKTARA | |
| | | IATQVGINKVFAEVLPSHKVAKVQELQNKGKKVAMVGDGVNDSPALA | |
| | | QADMGVAIGTGTDVAIEAADVVLIRNDLLDVVASIHLSKRTVRRIRI | |
| | | NLVLALIYNLVGIPIAAGVFMPIGIVLQPWMGSAAMAASSVSVVLSS | |
| | | LQLKCYKKPDLERYEAQAHGHMKPLTASQVSVHIGMDDRWRDSPRAT | |
| | | PWDQVSYVSQVSLSSLTSDKPSRHSAAADDDGDKWSLLLNGRDEEQY | |
| | | I | |
| miniA | 42 | MPEQERQITAREGASRKILSKLSLPTRAWEPAMKKSFAFDNVGYEGG | 119 |
| TP7B | | LDGLGPSSQPQKCFLQIKGMTCASCVSNIERNLQKEAGVLSVLVALM | |
| v_v2 | | AGKAEIKYDPEVIQPLEIAQFIQDLGFEAAVMEDYAGSDGNIELTIT | |
| | | GMTCASCVHNIESKLTRTNGITYASVALATSKALVKFDPEIIGPRDI | |
| | | IKIIEEIGFHASLAQRNPNAHHLDHKMEIKQWKKSFLCSLVFGIPVM | |
| | | ALMIYMLIPSNEPHQSMVLDHNIIPGLSILNLIFFILCTFVQLLGGW | |
| | | YFYVQAYKSLRHRSANMDVLIVLATSIAYVYSLVILVVAVAEKAERS | |
| | | PVTFFDTPPMLFVFIALGRWLEHLAKSKTSEALAKLMSLQATEATVV | |
| | | TLGEDNLIIREEQVPMELVQRGDIVRVVPGGKFPVDGKVLEGNTMAD | |
| | | ESLITGEAMPVTKKPGSTVIAGSINAHGSVLIKATHVGNDTTLAQIV | |
| | | KLVEEAQMSKAPIQQLADRFSGYFVPFIIIMSTLTLVVWIVIGFIDF | |
| | | GVVQKYFPNPNKHISQTEVIIRFAFQTSITVLCIACPCSLGLATPTA | |
| | | VMVGTGVAAQNGILIKGGKPLEMAHKIKTVMFDKTGTITHGVPRVMR | |
| | | VLLLGDVATLPLRKVLAVVGTAEASSEHPLGVAVTKYCKEELGTETL | |
| | | GYCTDFQAVPGCGIGCKVSNVEGILAHSERPLSAPASHLNEAGSLPA | |
| | | EKDAAPQTFSVLIGNREWLRRNGLTISSDVSDAMTDHEMKGQTAILV | |
| | | AIDGVLCGMIAIADAVKQEAALAVHTLQSMGVDVVLITGDNRKTARA | |
| | | IATQVGINKVFAEVLPSHKVAKVQELQNKGKKVAMVGDGVNDSPALA | |
| | | QADMGVAIGTGTDVAIEAADVVLIRNDLLDVVASIHLSKRTVRRIRI | |
| | | NLVLALIYNLVGIPIAAGVFMPIGIVLQPWMGSAAMAASSVSVVLSS | |
| | | LQLKCYKKPDLERYEAQAHGHMKPLTASQVSVHIGMDDRWRDSPRAT | |
| | | PWDQVSYVSQVSLSSLTSDKPSRHSAAADDDGDKWSLLLNGRDEEQY | |
| | | I | |
| TISU- | 48 | MAAPEQERQITAREGASRKILSKLSLPTRAWEPAMKKSFAFDNVGYE | 120 |
| miniA | | GGLDGLGPSSQPQKCFLQIKGMTCASCVSNIERNLQKEAGVLSVLVA | |
| TP7B | | LMAGKAEIKYDPEVIQPLEIAQFIQDLGFEAAVMEDYAGSDGNIELT | |
| V.co | | ITGMTCASCVHNIESKLTRTNGITYASVALATSKALVKFDPEIIGPR | |
| | | DIIKIIEEIGFHASLAQRNPNAHHLDHKMEIKQWKKSFLCSLVFGIP | |
| | | VMALMIYMLIPSNEPHQSMVLDHNIIPGLSILNLIFFILCTFVQLLG | |
| | | GWYFYVQAYKSLRHRSANMDVLIVLATSIAYVYSLVILVVAVAEKAE | |
| | | RSPVTFFDTPPMLFVFIALGRWLEHLAKSKTSEALAKLMSLQATEAT | |
| | | VVTLGEDNLIIREEQVPMELVQRGDIVKVVPGGKFPVDGKVLEGNTM | |
| | | ADESLITGEAMPVTKKPGSTVIAGSINAHGSVLIKATHVGNDTTLAQ | |
| | | IVKLVEEAQMSKAPIQQLADRFSGYFVPFIIIMSTLTLVVWIVIGFI | |
| | | DFGVVQRYFPNPNKHISQTEVIIRFAFQTSITVLCIACPCSLGLATP | |
| | | TAVMVGTGVAAQNGILIKGGKPLEMAHKIKTVMFDKTGTITHGVPRV | |
| | | MRVLLLGDVATLPLRKVLAVVGTAEASSEHPLGVAVTKYCKEELGTE | |
| | | TLGYCTDFQAVPGCGIGCKVSNVEGILAHSERPLSAPASHLNEAGSL | |
| | | PAEKDAVPQTFSVLIGNREWLRRNGLTISSDVSDAMTDHEMKGQTAI | |
| | | LVAIDGVLCGMIAIADAVKQEAALAVHTLQSMGVDVVLITGDNRKTA | |
| | | RAIATQVGINKVFAEVLPSHKVAKVQELQNKGKKVAMVGDGVNDSPA | |
| | | LAQADMGVAIGTGTDVAIEAADVVLIRNDLLDVVASIHLSKRTVRRI | |
| | | RINLVLALIYNLVGIPIAAGVFMPIGIVLQPWMGSAAMAASSVSVVL | |
| | | SSLQLKCYKKPDLERYEAQAHGHMKPLTASQVSVHIGMDDRWRDSPR | |
| | | ATPWDQVSYVSQVSLSSLTSDKPSRHSAAADDDGDKWSLLLNGRDEE | |
| | | QYI | |
| miniA | 45 | MPEQERQITAREGASRKILSKLSLPTRAWEPAMKKSFAFDNVGYEGG | 121 |
| TP7B- | | LDGLGPSSQVAPDGAEGSGTDHRSSSSHSPGSPPRNQVQGTCSKCFL | |
| WD6 | | QIKGMTCASCVSNIERNLQKEAGVLSVLVALMAGKAEIKYDPEVIQP | |
| | | LEIAQFIQDLGFEAAVMEDYAGSDGNIELTITGMTCASCVHNIESKL | |
| | | TRTNGITYASVALATSKALVKFDPEIIGPRDIIKIIEEIGFHASLAQ | |
| | | RNPNAHHLDHKMEIKQWKKSFLVSLFFCIPVMGLMIYMMVMDHHFAT | |
| | | LHHNQNMSKEEMINLHSSMFLERQILPGLSVMNLLSFLLCVPVQFFG | |
| | | GWYFYIQAYKALKHKTANMDVLIVLATTIAFAYSLIILLVAMYERAK | |
| | | VNPITFFDTPPMLFVFIALGRWLEHIAKGKTSEALAKLISLQATEAT | |
| | | IVTLDSDNILLSEEQVDVELVQRGDIIKVVPGGKFPVDGRVIEGHSM | |
| | | VDESLITGEAMPVAKKPGSTVIAGSINQNGSLLICATHVGADTTLSQ | |
| | | IVKLVEEAQTSKAPIQQFADKLSGYFVPFIVFVSIATLLVWIVIGFL | |
| | | NFEIVETYFPGYNRSISRTETIIRFAFQASITVLCIACPCSLGLATP | |
| | | TAVMVGTGVGAQNGILIKGGEPLEMAHKVKVVVFDKTGTITHGTPVV | |
| | | NQVKVLTESNRISHHKILAIVGTAESNSEHPLGTAITKYCKQELDTE | |
| | | TLGTCIDFQVVPGCGISCKVTNIEGLLHKNNWNIEDNNIKNASLVQI | |
| | | DASNEQSSTSSSMIIDAQISNALNAQQYKVLIGNREWMIRNGLVINN | |
| | | DVNDFMTEHERKGRTAVLVAVDDELCGLIAIADTVKPEAELAIHILK | |
| | | SMGLEVVLMTGDNSKTARSIASQVGITKVFAEVLPSHKVAKVKQLQE | |
| | | EGKRVAMVGDGINDSPALAMANVGIAIGTGTDVAIEAADVVLIRNDL | |
| | | LDVVASIDLSRKTVKRIRINFVFALIYNLVGIPIAAGVFMPIGLVLQ | |
| | | PWMGSAAMAASSVSVVLSSLQLKCYKKPDLERYEAQAHGHMKPLTAS | |
| | | QVSVHIGMDDRWRDSPRATPWDQVSYVSQVSLSSLTSDKPSRHSAAA | |
| | | DDDGDKWSLLLNGRDEEQYI | |
| miniA | 35 | MAAPEQERQITAREGASRKILSKLSLPTRAWEPAMKKSFAFDNVGYE | 122 |
| TP7A | | GGLDGLGPSSQVAPDGAEGSGTDHRSSSSHSPGSPPRNQVQGTCSKC | |
| B | | FLQIKGMTCASCVSNIERNLQKEAGVLSVLVALMAGKAEIKYDPEVI | |
| | | QPLEIAQFIQDLGFEAAVMEDYAGSDGNIELTITGMTCASCVHNIES | |
| | | KLTRTNGITYASVALATSKALVKFDPEIIGPRDIIKIIEEIGFHASL | |
| | | AQRNPNAHHLDHKMEIKQWKKSFLVSLFFCIPVMGLMIYMMVMDHHF | |
| | | ATLHHNQNMSKEEMINLHSSMFLERQILPGLSVMNLLSFLLCVPVQF | |
| | | FGGWYFYIQAYKALKHKTANMDVLIVLATTIAFAYSLIILLVAMYER | |
| | | AKVNPITFFDTPPMLFVFIALGRWLEHIAKGKTSEALAKLISLQATE | |
| | | ATIVTLDSDNILLSEEQVDVELVQRGDIIKVVPGGKFPVDGRVIEGH | |
| | | SMVDESLITGEAMPVAKKPGSTVIAGSINQNGSLLICATHVGADTTL | |
| | | SQIVKLVEEAQTSKAPIQQFADKLSGYFVPFIVFVSIATLLVWIVIG | |
| | | FLNFEIVETYFPGYNRSISRTETIIRFAFQASITVLCIACPCSLGLA | |
| | | TPTAVMVGTGVGAQNGILIKGGEPLEMAHKVKVVVFDKTGTITHGTP | |
| | | VVNQVKVLTESNRISHHKILAIVGTAESNSEHPLGTAITKYCKQELD | |
| | | TETLGTCIDFQVVPGCGISCKVTNIEGLLHKNNWNIEDNNIKNASLV | |
| | | QIDASNEQSSTSSSMIIDAQISNALNAQQYKVLIGNREWMIRNGLVI | |
| | | NNDVNDFMTEHERKGRTAVLVAVDDELCGLIAIADTVKPEAELAIHI | |
| | | LKSMGLEVVLMTGDNSKTARSIASQVGITKVFAEVLPSHKVAKVKQL | |
| | | QEEGKRVAMVGDGINDSPALAMANVGIAIGTGTDVAIEAADVVLIRN | |
| | | DLLDVVASIDLSRKTVKRIRINFVFALIYNLVGIPIAAGVFMPIGLV | |
| | | LQPWMGSAAMAASSVSVVLSSLQLKCYKKPDLERYEAQAHGHMKPLT | |
| | | ASQVSVHIGMDDRWRDSPRATPWDQVSYVSQVSLSSLTSDKPSRHSA | |
| | | AADDDGDKWSLLLNGRDEEQYI | |
| miniA | 39 | MPEQERQITAREGASRKILSKLSLPTRAWEPAMKKSFAFDNVGYEGG | 123 |
| TP7B- | | LDGLGPSSQVATTLIAIAGMTCASCVHSIEGMISQLEGVQQISVSLA | |
| slco3 | | EGTATVLYNPSVISPEELRAAIEDMGFEASVVSESCSTNPLGN | |
| miniA | 37 | HSAGNSMVQTTDGTPTSVQEVAPHTGRLPANHAPDILAKSPQSTRAV | 123 |
| TP7B- | | APQKCFLQIKGMTCASCVSNIERNLQKEAGVLSVLVALMAGKAEIKY | |
| slco | | DPEVIQPLEIAQFIQDLGFEAAVMEDYAGSDGNIELTITGM | |
| miniA | 38 | TCASCVHNIESKLTRINGITYASVALATSKALVKFDPEIIGPRDIIK | 123 |
| TP7B- | | IIEEIGFHASLAQRNPNAHHLDHKMEIKQWKKSFLCSLVFGIPVMAL | |
| slco2 | | MIYMLIPSNEPHQSMVLDHNIIPGLSILNLIFFILCTFVQLLGGWY | |
| miniA | 36 | FYVQAYKSLRHRSANMDVLIVLATSIAYVYSLVILVVAVAEKAERSP | 123 |
| TP7B- | | VTFFDTPPMLFVFIALGRWLEHLAKSKTSEALAKLMSLQATEATVVT | |
| S1 | | LGEDNLIIREEQVPMELVQRGDIVKVVPGGKFPVDGKVLEGNTMADE | |
| | | SLITGEAMPVTKKPGSTVIAGSINAHGSVLIKATHVGNDTTLAQIVK | |
| | | LVEEAQMSKAPIQQLADRFSGYFVPFIIIMSTLTLVVWIVIGFIDFG | |
| | | VVQRYFPNPNKHISQTEVIIRFAFQTSITVLCIACPCSLGLATPTAV | |
| | | MVGTGVAAQNGILIKGGKPLEMAHKIKTVMFDKTGTITHGVPRVMRV | |
| | | LLLGDVATLPLRKVLAVVGTAEASSEHPLGVAVTKYCKEELGTETLG | |
| | | YCTDFQAVPGCGIGCKVSNVEGILAHSERPLSAPASHLNEAGSLPAE | |
| | | KDAVPQTFSVLIGNREWLRRNGLTISSDVSDAMTDHEMKGQTAILVA | |
| | | IDGVLCGMIAIADAVKQEAALAVHTLQSMGVDVVLITGDNRKTARAI | |
| | | ATQVGINKVFAEVLPSHKVAKVQELQNKGKKVAMVGDGVNDSPALAQ | |
| | | ADMGVAIGTGTDVAIEAADVVLIRNDLLDVVASIHLSKRTVRRIRIN | |
| | | LVLALIYNLVGIPIAAGVFMPIGIVLQPWMGSAAMAASSVSVVLSSL | |
| | | QLKCYKKPDLERYEAQAHGHMKPLTASQVSVHIGMDDRWRDSPRATP | |
| | | WDQVSYVSQVSLSSLTSDKPSRHSAAADDDGDKWSLLLNGRDEEQYI | |
| TISU- | 46 | MAAPEQERQITAREGASRKILSKLSLPTRAWEPAMKKSFAFDNVGYE | 124 |
| miniA | | GGLDGLGPSSQVATTLIAIAGMTCASCVHSIEGMISQLEGVQQISVS | |
| TP7B- | | LAEGTATVLYNPSVISPEELRAAIEDMGFEASVVSESCSTNPLGNHS | |
| slco | | AGNSMVQTTDGTPTSVQEVAPHTGRLPANHAPDILAKSPQS | |
| TISU- | 47 | TRAVAPQKCFLQIKGMTCASCVSNIERNLQKEAGVLSVLVALMAGKA | 124 |
| miniA | | EIKYDPEVIQPLEIAQFIQDLGFEAAVMEDYAGSDGNIELTITGMTC | |
| TP7B- | | ASCVHNIESKLTRTNGITYASVALATSKALVKFDPEIIGPRDIIKII | |
| slco3 | | EEIGFHASLAQRNPNAHHLDHKMEIKQWKKSFLCSLVFGIPVMALMI | |
| | | YMLIPSNEPHQSMVLDHNIIPGLSILNLIFFILCTFVQLLGGWYFYV | |
| | | QAYKSLRHRSANMDVLIVLATSIAYVYSLVILVVAVAEKAERSPVTF | |
| | | FDTPPMLFVFIALGRWLEHLAKSKTSEALAKLMSLQATEATVVTLGE | |
| | | DNLIIREEQVPMELVQRGDIVKVVPGGKFPVDGKVLEGNTMADESLI | |
| | | TGEAMPVTKKPGSTVIAGSINAHGSVLIKATHVGNDTTLAQIVKLVE | |
| | | EAQMSKAPIQQLADRFSGYFVPFIIIMSTLTLVVWIVIGFIDFGVVQ | |
| | | RYFPNPNKHISQTEVIIRFAFQTSITVLCIACPCSLGLATPTAVMVG | |
| | | TGVAAQNGILIKGGKPLEMAHKIKTVMFDKTGTITHGVPRVMRVLLL | |
| | | GDVATLPLRKVLAVVGTAEASSEHPLGVAVTKYCKEELGTETLGYCT | |
| | | DFQAVPGCGIGCKVSNVEGILAHSERPLSAPASHLNEAGSLPAEKDA | |
| | | VPQTFSVLIGNREWLRRNGLTISSDVSDAMTDHEMKGQTAILVAIDG | |
| | | VLCGMIAIADAVKQEAALAVHTLQSMGVDVVLITGDNRKTARAIATQ | |
| | | VGINKVFAEVLPSHKVAKVQELQNKGKKVAMVGDGVNDSPALAQADM | |
| | | GVAIGTGTDVAIEAADVVLIRNDLLDVVASIHLSKRTVRRIRINLVL | |
| | | ALIYNLVGIPIAAGVFMPIGIVLQPWMGSAAMAASSVSVVLSSLQLK | |
| | | CYKKPDLERYEAQAHGHMKPLTASQVSVHIGMDDRWRDSPRATPWDQ | |
| | | VSYVSQVSLSSLTSDKPSRHSAAADDDGDKWSLLLNGRDEEQYI | |
| miniA | 43 | MPEQERQITAREGASRKILSKLSLPTRAWEPAMKKSFAFDNVGYEGG | 125 |
| TP7B- | | LDGLGPSSQVATSTVRILGMTCQSCVKSIEDRISNLKGIISMKVSLE | |
| WD4 | | QGSATVKYVPSVVCLQQVCHQIGDMGFEASIPDGAEGSGTDHRSSSS | |
| | | HSPGSPPRNQVQGTCSKCFLQIKGMTCASCVSNIERNLQKEAGVLSV | |
| | | LVALMAGKAEIKYDPEVIQPLEIAQFIQDLGFEAAVMEDYAGSDGNI | |
| | | ELTITGMTCASCVHNIESKLTRTNGITYASVALATSKALVKFDPEII | |
| | | GPRDIIKIIEEIGFHASLAQRNPNAHHLDHKMEIKQWKKSFLVSLFF | |
| | | CIPVMGLMIYMMVMDHHFATLHHNQNMSKEEMINLHSSMFLERQILP | |
| | | GLSVMNLLSFLLCVPVQFFGGWYFYIQAYKALKHKTANMDVLIVLAT | |
| | | TIAFAYSLIILLVAMYERAKVNPITFFDTPPMLFVFIALGRWLEHIA | |
| | | KGKTSEALAKLISLQATEATIVTLDSDNILLSEEQVDVELVQRGDII | |
| | | KVVPGGKFPVDGRVIEGHSMVDESLITGEAMPVAKKPGSTVIAGSIN | |
| | | QNGSLLICATHVGADTTLSQIVKLVEEAQTSKAPIQQFADKLSGYFV | |
| | | PFIVFVSIATLLVWIVIGFLNFEIVETYFPGYNRSISRTETIIRFAF | |
| | | QASITVLCIACPCSLGLATPTAVMVGTGVGAQNGILIKGGEPLEMAH | |
| | | KVKVVVFDKTGTITHGTPVVNQVKVLTESNRISHHKILAIVGTAESN | |
| | | SEHPLGTAITKYCKQELDTETLGTCIDFQVVPGCGISCKVTNIEGLL | |
| | | HKNNWNIEDNNIKNASLVQIDASNEQSSTSSSMIIDAQISNALNAQQ | |
| | | YKVLIGNREWMIRNGLVINNDVNDFMTEHERKGRTAVLVAVDDELCG | |
| | | LIAIADTVKPEAELAIHILKSMGLEVVLMTGDNSKTARSIASQVGIT | |
| | | KVFAEVLPSHKVAKVKQLQEEGKRVAMVGDGINDSPALAMANVGIAI | |
| | | GTGTDVAIEAADVVLIRNDLLDVVASIDLSRKTVKRIRINFVFALIY | |
| | | NLVGIPIAAGVFMPIGLVLQPWMGSAAMAASSVSVVLSSLQLKCYKK | |
| | | PDLERYEAQAHGHMKPLTASQVSVHIGMDDRWRDSPRATPWDQVSYV | |
| | | SQVSLSSLTSDKPSRHSAAADDDGDKWSLLLNGRDEEQYI | |
| miniA | 40 | MPEQERQITAREGASRKILSKLSLPTRAWEPAMKKSFAFDNVGYEGG | 126 |
| TP7B- | | LDGLGPSSQVAPDGAEGSGTDHRSSSSHSPGSPPRNQVQGTCSTTLI | |
| s6 | | AIAGMTCASCVHSIEGMISQLEGVQQISVSLAEGTATVLYNPSVISP | |
| | | EELRAAIEDMGFEASVVSESCSTNPLGNHSAGNSMVQTTDGTPTSVQ | |
| | | EVAPHTGRLPANHAPDILAKSPQSTRAVAPQKCFLQIKGMTCASCVS | |
| | | NIERNLQKEAGVLSVLVALMAGKAEIKYDPEVIQPLEIAQFIQDLGF | |
| | | EAAVMEDYAGSDGNIELTITGMTCASCVHNIESKLTRTNGITYASVA | |
| | | LATSKALVKFDPEIIGPRDIIKIIEEIGFHASLAQRNPNAHHLDHKM | |
| | | EIKQWKKSFLCSLVFGIPVMALMIYMLIPSNEPHQSMVLDHNIIPGL | |
| | | SILNLIFFILCTFVQLLGGWYFYVQAYKSLRHRSANMDVLIVLATSI | |
| | | AYVYSLVILVVAVAEKAERSPVTFFDTPPMLFVFIALGRWLEHLAKS | |
| | | KTSEALAKLMSLQATEATVVTLGEDNLIIREEQVPMELVQRGDIVKV | |
| | | VPGGKFPVDGKVLEGNTMADESLITGEAMPVTKKPGSTVIAGSINAH | |
| | | GSVLIKATHVGNDTTLAQIVKLVEEAQMSKAPIQQLADRFSGYFVPF | |
| | | IIIMSTLTLVVWIVIGFIDFGVVQRYFPNPNKHISQTEVIIRFAFQT | |
| | | SITVLCIACPCSLGLATPTAVMVGTGVAAQNGILIKGGKPLEMAHKI | |
| | | KTVMFDKTGTITHGVPRVMRVLLLGDVATLPLRKVLAVVGTAEASSE | |
| | | HPLGVAVTKYCKEELGTETLGYCTDFQAVPGCGIGCKVSNVEGILAH | |
| | | SERPLSAPASHLNEAGSLPAEKDAVPQTFSVLIGNREWLRRNGLTIS | |
| | | SDVSDAMTDHEMKGQTAILVAIDGVLCGMIAIADAVKQEAALAVHTL | |
| | | QSMGVDVVLITGDNRKTARAIATQVGINKVFAEVLPSHKVAKVQELQ | |
| | | NKGKKVAMVGDGVNDSPALAQADMGVAIGTGTDVAIEAADVVLIRND | |
| | | LLDVVASIHLSKRTVRRIRINLVLALIYNLVGIPIAAGVFMPIGIVL | |
| | | QPWMGSAAMAASSVSVVLSSLQLKCYKKPDLERYEAQAHGHMKPLTA | |
| | | SQVSVHIGMDDRWRDSPRATPWDQVSYVSQVSLSSLTSDKPSRHSAA | |
| | | ADDDGDKWSLLLNGRDEEQYI | |
| miniA | 44 | MPEQERQITAREGASRKILSKLSLPTRAWEPAMKKSFAFDNVGYEGG | 127 |
| TP7B- | | LDGLGPSSQVAPDGAEGSGTDHRSSSSHSPGSPPRNQVQGTCSTTLI | |
| WD5 | | AIAGMTCASCVHSIEGMISQLEGVQQISVSLAEGTATVLYNPSVISP | |
| | | EELRAAIEDMGFEASVVSESCSTNPLGNHSAGNSMVQTTDGTPTSVQ | |
| | | EVAPHTGRLPANHAPDILAKSPQSTRAVAPQKCFLQIKGMTCASCVS | |
| | | NIERNLQKEAGVLSVLVALMAGKAEIKYDPEVIQPLEIAQFIQDLGF | |
| | | EAAVMEDYAGSDGNIELTITGMTCASCVHNIESKLTRTNGITYASVA | |
| | | LATSKALVKFDPEIIGPRDIIKIIEEIGFHASLAQRNPNAHHLDHKM | |
| | | EIKQWKKSFLVSLFFCIPVMGLMIYMMVMDHHFATLHHNQNMSKEEM | |
| | | INLHSSMFLERQILPGLSVMNLLSFLLCVPVQFFGGWYFYIQAYKAL | |
| | | KHKTANMDVLIVLATTIAFAYSLIILLVAMYERAKVNPITFFDTPPM | |
| | | LFVFIALGRWLEHIAKGKTSEALAKLISLQATEATIVTLDSDNILLS | |
| | | EEQVDVELVQRGDIIKVVPGGKFPVDGRVIEGHSMVDESLITGEAMP | |
| | | VAKKPGSTVIAGSINQNGSLLICATHVGADTTLSQIVKLVEEAQTSK | |
| | | APIQQFADKLSGYFVPFIVFVSIATLLVWIVIGFLNFEIVETYFPGY | |
| | | NRSISRTETIIRFAFQASITVLCIACPCSLGLATPTAVMVGTGVGAQ | |
| | | NGILIKGGEPLEMAHKVKVVVFDKTGTITHGTPVVNQVKVLTESNRI | |
| | | SHHKILAIVGTAESNSEHPLGTAITKYCKQELDTETLGTCIDFQVVP | |
| | | GCGISCKVTNIEGLLHKNNWNIEDNNIKNASLVQIDASNEQSSTSSS | |
| | | MIIDAQISNALNAQQYKVLIGNREWMIRNGLVINNDVNDFMTEHERK | |
| | | GRTAVLVAVDDELCGLIAIADTVKPEAELAIHILKSMGLEVVLMTGD | |
| | | NSKTARSIASQVGITKVFAEVLPSHKVAKVKQLQEEGKRVAMVGDGI | |
| | | NDSPALAMANVGIAIGTGTDVAIEAADVVLIRNDLLDVVASIDLSRK | |
| | | TVKRIRINFVFALIYNLVGIPIAAGVFMPIGLVLQPWMGSAAMAASS | |
| | | VSVVLSSLQLKCYKKPDLERYEAQAHGHMKPLTASQVSVHIGMDDRW | |
| | | RDSPRATPWDQVSYVSQVSLSSLTSDKPSRHSAAADDDGDKWSLLLN | |
| | | GRDEEQYI | |
| ATP7 | 34 | MPEQERQITAREGASRKILSKLSLPTRAWEPAMKKSFAFDNVGYEGG | 128 |
| B_FL | | LDGLGPSSQVATSTVRILGMTCQSCVKSIEDRISNLKGIISMKVSLE | |
| | | QGSATVKYVPSVVCLQQVCHQIGDMGFEASIAEGKAASWPSRSLPAQ | |
| | | EAVVKLRVEGMTCQSCVSSIEGKVRKLQGVVRVKVSLSNQEAVITYQ | |
| | | PYLIQPEDLRDHVNDMGFEAAIKSKVAPLSLGPIDIERLQSTNPKRP | |
| | | LSSANQNFNNSETLGHQGSHVVTLQLRIDGMHCKSCVLNIEENIGQL | |
| | | LGVQSIQVSLENKTAQVKYDPSCTSPVALQRAIEALPPGNFKVSLPD | |
| | | GAEGSGTDHRSSSSHSPGSPPRNQVQGTCSTTLIAIAGMTCASCVHS | |
| | | IEGMISQLEGVQQISVSLAEGTATVLYNPAVISPEELRAAIEDMGFE | |
| | | ASVVSESCSTNPLGNHSAGNSMVQTTDGTPTSLQEVAPHTGRLPANH | |
| | | APDILAKSPQSTRAVAPQKCFLQIKGMTCASCVSNIERNLQKEAGVL | |
| | | SVLVALMAGKAEIKYDPEVIQPLEIAQFIQDLGFEAAVMEDYAGSDG | |
| | | NIELTITGMTCASCVHNIESKLTRTNGITYASVALATSKALVKFDPE | |
| | | IIGPRDIIKIIEEIGFHASLAQRNPNAHHLDHKMEIKQWKKSFLCSL | |
| | | VFGIPVMALMIYMLIPSNEPHQSMVLDHNIIPGLSILNLIFFILCTF | |
| | | VQLLGGWYFYVQAYKSLRHRSANMDVLIVLATSIAYVYSLVILVVAV | |
| | | AEKAERSPVTFFDTPPMLFVFIALGRWLEHLAKSKTSEALAKLMSLQ | |
| | | ATEATVVTLGEDNLIIREEQVPMELVQRGDIVRVVPGGKFPVDGKVL | |
| | | EGNTMADESLITGEAMPVTKKPGSTVIAGSINAHGSVLIKATHVGND | |
| | | TTLAQIVKLVEEAQMSKAPIQQLADRFSGYFVPFIIIMSTLTLVVWI | |
| | | VIGFIDFGVVQKYFPNPNKHISQTEVIIRFAFQTSITVLCIACPCSL | |
| | | GLATPTAVMVGTGVAAQNGILIKGGKPLEMAHKIKTVMFDKTGTITH | |
| | | GVPRVMRVLLLGDVATLPLRKVLAVVGTAEASSEHPLGVAVTKYCKE | |
| | | ELGTETLGYCTDFQAVPGCGIGCKVSNVEGILAHSERPLSAPASHLN | |
| | | EAGSLPAEKDAAPQTFSVLIGNREWLRRNGLTISSDVSDAMTDHEMK | |
| | | GQTAILVAIDGVLCGMIAIADAVKQEAALAVHTLQSMGVDVVLITGD | |
| | | NRKTARAIATQVGINKVFAEVLPSHKVAKVQELQNKGKKVAMVGDGV | |
| | | NDSPALAQADMGVAIGTGTDVAIEAADVVLIRNDLLDVVASIHLSKR | |
| | | TVRRIRINLVLALIYNLVGIPIAAGVFMPIGIVLQPWMGSAAMAASS | |
| | | VSVVLSSLQLKCYKKPDLERYEAQAHGHMKPLTASQVSVHIGMDDRW | |
| | | RDSPRATPWDQVSYVSQVSLSSLTSDKPSRHSAAADDDGDKWSLLLN | |
| | | GRDEEQYI | |

We claim:

1. An expression construct comprising:
(a) a promoter;
(b) a sequence encoding (ATPase Copper Transporting Beta) ATP7B, operatively linked to the promoter; and
(c) a polyadenylation signal.

2. The expression construct of claim 1, wherein the promoter comprises a sequence that is at least 80% identical to a sequence selected from the group consisting of SEQ ID NOs: 4-33.

3. The expression construct of claim 2, wherein the promoter comprises a sequence that is at least 90% identical to a sequence selected from the group consisting of SEQ ID NOs: 4-33.

4. The expression construct of claim 3, wherein the promoter comprises a sequence that is at least 95% identical to a sequence selected from the group consisting of SEQ ID NOs: 4-33.

5. The expression construct of claim 4, wherein the promoter comprises a sequence selected from the group consisting of SEQ ID NOs: 4-33.

6. The expression construct of claim 1, wherein the promoter comprises a sequence that is at least 80% identical to any one of SEQ ID NO:4, SEQ ID NO: 10, or SEQ ID NO:11.

7. The expression construct of claim 6, wherein the promoter comprises a sequence that is at least 90% identical to any one of SEQ ID NO:4, SEQ ID NO: 10, or SEQ ID NO:11.

8. The expression construct of claim 7, wherein the promoter comprises a sequence that is at least 95% identical to any one of SEQ ID NO:4, SEQ ID NO:10, or SEQ ID NO: 11.

9. The expression construct of claim 8, wherein the promoter comprises any one of SEQ ID NO: 4, SEQ ID NO: 10, or SEQ ID NO:11.

10. The expression construct of any one of claims 1-8, wherein the sequence encoding ATP7B is codon-optimized.

11. The expression construct of any one of claims 1-8, wherein the sequence encoding ATP7B comprises a sequence that is at least 80% identical to any one of SEQ ID NOs: 35-48.

12. The expression construct of claim 11, wherein the sequence encoding ATP7B comprises a sequence that is at least 90% identical to any one of SEQ ID NOs: 35-48.

13. The expression construct of claim 12, wherein the sequence encoding ATP7B comprises a sequence that is at least 95% identical to any one of SEQ ID NOs: 35-48.

14. The expression construct of claim 13, wherein the sequence encoding ATP7B comprises a sequence selected from the group consisting of SEQ ID NOs: 35-48.

15. The expression construct of any one of claims 1-8, wherein the sequence encoding ATP7B comprises a sequence that is at least 80% identical to SEQ ID NO:39 or SEQ ID NO:41.

16. The expression construct of claim 15, wherein the sequence encoding ATP7B comprises a sequence that is at least 90% identical to SEQ ID NO:39 or SEQ ID NO:41.

17. The expression construct of claim 16, wherein the sequence encoding ATP7B comprises a sequence that is at least 95% identical to SEQ ID NO:39 or SEQ ID NO:41.

18. The expression construct of claim 17, wherein the sequence encoding ATP7B comprises SEQ ID NO:39 or SEQ ID NO:41.

19. The expression construct of any of the preceding claims, wherein the sequence encoding ATP7B encodes a protein comprising a sequence that is at least 80% identical to any one of SEQ ID NOs: 118-128.

20. The expression construct of claim 19, wherein the sequence encoding ATP7B encodes a protein comprising a sequence that is at least 90% identical to any one of SEQ ID NOs: 118-128.

21. The expression construct of claim 20, wherein the sequence encoding ATP7B encodes a protein comprising a sequence that is at least 95% identical to any one of SEQ ID NOs: 118-128.

22. The expression construct of claim 21, wherein the sequence encoding ATP7B encodes a protein comprising any one of SEQ ID NOs: 118-128.

23. The expression construct of claim 19, wherein the sequence encoding ATP7B encodes a protein comprising a sequence that is at least 80% identical to SEQ ID NO:118 or SEQ ID NO: 123.

24. The expression construct of claim 23, wherein the sequence encoding ATP7B encodes a protein comprising a sequence that is at least 90% identical to SEQ ID NO:118 or SEQ ID NO: 123.

25. The expression construct of claim 24, wherein the sequence encoding ATP7B encodes a protein comprising a sequence that is at least 95% identical to SEQ ID NO:118 or SEQ ID NO: 123.

26. The expression construct of claim 25, wherein the sequence encoding ATP7B encodes a protein comprising SEQ ID NO:118 or SEQ ID NO:123.

27. The expression construct of any of the preceding claims, wherein the expression construct further comprises a post-transcriptional regulatory element.

28. The expression construct of claim 27, wherein the post-transcriptional regulatory element comprises a sequence that is at least 80% identical to SEQ ID NO:49 or SEQ ID NO:50.

29. The expression construct of claim 28, wherein the post-transcriptional regulatory element comprises a sequence that is at least 90% identical to SEQ ID NO:49 or SEQ ID NO:50.

30. The expression construct of claim 29, wherein the post-transcriptional regulatory element comprises a sequence that is at least 95% identical to SEQ ID NO:49 or SEQ ID NO:50.

31. The expression construct of claim 30, wherein the post-transcriptional regulatory element comprises SEQ ID NO:49 or SEQ ID NO:50.

32. The expression construct of claim 27, wherein the post-transcriptional regulatory element comprises a sequence that is at least 80% identical to SEQ ID NO:49.

33. The expression construct of claim 32, wherein the post-transcriptional regulatory element comprises a sequence that is at least 90% identical to SEQ ID NO:49.

34. The expression construct of claim 33, wherein the post-transcriptional regulatory element comprises a sequence that is at least 95% identical to SEQ ID NO:49.

35. The expression construct of claim 34, wherein the post-transcriptional regulatory element comprises SEQ ID NO:49.

36. The expression construct of any one of claims 1-35, wherein the polyadenylation signal comprises a sequence that is at least 80% identical to SEQ ID NO:51 or SEQ ID NO:52.

37. The expression construct of claim 36, wherein the polyadenylation signal comprises a sequence that is at least 90% identical to SEQ ID NO:51 or SEQ ID NO:52.

38. The expression construct of claim 37, wherein the polyadenylation signal comprises a sequence that is at least 95% identical to SEQ ID NO:51 or SEQ ID NO:52.

39. The expression construct of claim 38, wherein the polyadenylation signal comprises SEQ ID NO: 51 or SEQ ID NO:52.

40. The expression construct of any one of claims 1-35, wherein the polyadenylation signal comprises a sequence that is at least 80% identical to SEQ ID NO:51.

41. The expression construct of claim 40, wherein the polyadenylation signal comprises a sequence that is at least 90% identical to SEQ ID NO:51.

42. The expression construct of claim 41, wherein the polyadenylation signal comprises a sequence that is at least 95% identical to SEQ ID NO:51.

43. The expression construct of claim 42, wherein the polyadenylation signal comprises SEQ ID NO: 51.

44. The expression construct of any one of the preceding claims, wherein the expression construct further comprises a miRNA binding site (miRBS).

45. The expression construct of claim 44, wherein the miRBS comprises a sequence that is at least 80% identical to SEQ ID NO:53.

46. The expression construct of claim 45, wherein the miRBS comprises a sequence that is at least 90% identical to SEQ ID NO:53.

47. The expression construct of claim 46, wherein the miRBS comprises a sequence that is at least 95% identical to SEQ ID NO:53.

48. The expression construct of claim 47, wherein the miRBS comprises SEQ ID NO:53.

49. The expression construct of claim 1, wherein the expression construct comprises:
(a) a promoter comprising a sequence that is at least 80% identical to SEQ ID NO:11;
(b) a sequence encoding ATP7B, operatively linked to the promoter, comprising a sequence that is at least 80% identical to SEQ ID NO:41;
(c) a post-transcriptional regulatory element comprising a sequence that is at least 80% identical to SEQ ID NO:49; and
(d) a polyadenylation signal comprising a sequence that is at least 80% identical to SEQ ID NO: 51.

50. The expression construct of claim 49, wherein the expression construct comprises:
(a) a promoter comprising a sequence that is at least 90% identical to SEQ ID NO:11;
(b) a sequence encoding ATP7B, operatively linked to the promoter, comprising a sequence that is at least 90% identical to SEQ ID NO:41;
(c) a post-transcriptional regulatory element comprising a sequence that is at least 90% identical to SEQ ID NO:49; and
(d) a polyadenylation signal comprising a sequence that is at least 90% identical to SEQ ID NO: 51.

51. The expression construct of claim 50, wherein the expression construct comprises:
(a) a promoter comprising a sequence that is at least 95% identical to SEQ ID NO:11;
(b) a sequence encoding ATP7B, operatively linked to the promoter, comprising a sequence that is at least 95% identical to SEQ ID NO:41;
(c) a post-transcriptional regulatory element comprising a sequence that is at least 95% identical to SEQ ID NO:49; and
(d) a polyadenylation signal comprising a sequence that is at least 95% identical to SEQ ID NO: 51.

52. The expression construct of claim 1, wherein the expression construct comprises:
(a) a promoter comprising SEQ ID NO:11;
(b) a sequence encoding ATP7B, operatively linked to the promoter, comprising SEQ ID NO: 41;
(c) a post-transcriptional regulatory element comprising SEQ ID NO:49; and
(d) a polyadenylation signal comprising SEQ ID NO:51.

53. The expression construct of claim 1, wherein the expression construct comprises:
(a) a promoter comprising a sequence that is at least 80% identical to SEQ ID NO:10;
(b) a sequence encoding ATP7B, operatively linked to the promoter, comprising a sequence that is at least 80% identical to SEQ ID NO:39;
(c) a miRBS comprising a sequence that is at least 80% identical to SEQ ID NO:53;
(d) a post-transcriptional regulatory element comprising a sequence that is at least 80% identical to SEQ ID NO:49; and
(e) a polyadenylation signal comprising a sequence that is at least 80% identical to SEQ ID NO: 51.

54. The expression construct of claim 53, wherein the expression construct comprises:
(a) a promoter comprising a sequence that is at least 90% identical to SEQ ID NO:10;
(b) a sequence encoding ATP7B, operatively linked to the promoter, comprising a sequence that is at least 90% identical to SEQ ID NO:39;
(c) a miRBS comprising a sequence that is at least 90% identical to SEQ ID NO:53;
(d) a post-transcriptional regulatory element comprising a sequence that is at least 90% identical to SEQ ID NO:49; and
(e) a polyadenylation signal comprising a sequence that is at least 90% identical to SEQ ID NO: 51.

55. The expression construct of claim 54, wherein the expression construct comprises:
(a) a promoter comprising a sequence that is at least 95% identical to SEQ ID NO:10;
(b) a sequence encoding ATP7B, operatively linked to the promoter, comprising a sequence that is at least 95% identical to SEQ ID NO:39;
(c) a miRBS comprising a sequence that is at least 95% identical to SEQ ID NO:53; and
(d) a post-transcriptional regulatory element comprising a sequence that is at least 95% identical to SEQ ID NO:49; and
(e) a polyadenylation signal comprising a sequence that is at least 95% identical to SEQ ID NO: 51.

56. The expression construct of claim 1, wherein the expression construct comprises:
(a) a promoter comprising SEQ ID NO:10;
(b) a sequence encoding ATP7B, operatively linked to the promoter, comprising SEQ ID NO: 39;
(c) a miRBS comprising SEQ ID NO:53;
(d) a post-transcriptional regulatory element comprising SEQ ID NO:49; and
(e) a polyadenylation signal comprising SEQ ID NO:51.

57. The expression construct of claim 1, wherein the expression construct comprises:
(a) a promoter comprising a sequence that is at least 80% identical to SEQ ID NO:4;
(b) a sequence encoding ATP7B, operatively linked to the promoter, comprising a sequence that is at least 80% identical to SEQ ID NO:41;
(c) a post-transcriptional regulatory element comprising a sequence that is at least 80% identical to SEQ ID NO:49; and
(d) a polyadenylation signal comprising a sequence that is at least 80% identical to SEQ ID NO: 51.

58. The expression construct of claim 57, wherein the expression construct comprises:
(a) a promoter comprising a sequence that is at least 90% identical to SEQ ID NO:4;
(b) a sequence encoding ATP7B, operatively linked to the promoter, comprising a sequence that is at least 90% identical to SEQ ID NO:41;
(c) a post-transcriptional regulatory element comprising a sequence that is at least 90% identical to SEQ ID NO:49; and
(d) a polyadenylation signal comprising a sequence that is at least 90% identical to SEQ ID NO: 51.

59. The expression construct of claim 58, wherein the expression construct comprises:
(a) a promoter comprising a sequence that is at least 95% identical to SEQ ID NO:4;
(b) a sequence encoding ATP7B, operatively linked to the promoter, comprising a sequence that is at least 95% identical to SEQ ID NO:41;
(c) a post-transcriptional regulatory element comprising a sequence that is at least 95% identical to SEQ ID NO:49; and
(d) a polyadenylation signal comprising a sequence that is at least 95% identical to SEQ ID NO: 51.

60. The expression construct of claim 59, wherein the expression construct comprises:
(a) a promoter comprising SEQ ID NO:4;
(b) a sequence encoding ATP7B, operatively linked to the promoter, comprising SEQ ID NO: 41;
(c) a post-transcriptional regulatory element SEQ ID NO:49; and
(d) a polyadenylation signal comprising SEQ ID NO:51.

61. A vector comprising the expression construct of any one of claims 1-60.

62. The vector of claim 57, wherein the vector is a viral vector.

63. The vector of claim 62, wherein the vector is an AAV vector.

64. A vector comprising a nucleic acid sequence comprising (i) the expression construct of any one of claims 1-60 and (ii) one or more inverted terminal repeats (ITR).

65. The vector of claim 64, wherein the nucleic acid sequence comprises a 5′ ITR and a 3′ ITR.

66. The vector of claim 65, wherein the 5′ ITR and the 3′ ITR are derived from adeno-associated virus (AAV) serotype AAV2.

67. The vector of claim 65, wherein the sequence of the 5′ ITR is at least 80% identical to SEQ ID NO: 116.

68. The vector of claim 66, wherein the sequence of the 5′ ITR is at least 90% identical to SEQ ID NO: 116.

69. The vector of claim 67, wherein the sequence of the 5′ ITR is at least 95% identical to SEQ ID NO: 116.

70. The vector of claim 69, wherein the sequence of the 5′ ITR comprises SEQ ID NO:116.

71. The vector of any one of claim 65 or 67-70, wherein the sequence of the 3′ ITR is at least 80% identical to SEQ ID NO: 117.

72. The vector of claim 71, wherein the sequence of the 3′ ITR is at least 90% identical to SEQ ID NO: 117.

73. The vector of claim 72, wherein the sequence of the 3′ ITR is at least 95% identical to SEQ ID NO: 117.

74. The vector of claim 73, wherein the sequence of the 3′ ITR comprises SEQ ID NO: 117.

75. A vector comprising the expression construct of claim 1, wherein the expression construct comprises a sequence that is least 80% identical to any one of SEQ ID NOs: 54-115.

76. The vector of claim 75, wherein the vector comprises a sequence that is least 90% identical to any one of SEQ ID NOs: 54-115.

77. The vector of claim 76, wherein the vector comprises a sequence that is least 95% identical to any one of SEQ ID NOs: 54-115.

78. The vector of claim 77, wherein the vector comprises any one of SEQ ID NOs: 54-115.

79. A vector comprising the expression construct of claim 1, wherein the vector comprises a sequence that is least 80% identical to any one of SEQ ID NO:65, SEQ ID NO:73, or SEQ ID NO: 92.

80. The vector of claim 79, wherein the vector comprises a sequence that is least 90% identical to any one of SEQ ID NO:65, SEQ ID NO: 73, or SEQ ID NO:92.

81. The vector of claim 80, wherein the vector comprises a sequence that is least 95% identical to SEQ ID NO:65, SEQ ID NO: 73, or SEQ ID NO:92.

82. The vector of claim 81, wherein the vector comprises SEQ ID NO:65, SEQ ID NO:73, or SEQ ID NO:92.

83. The vector of any one of claims 57-82, wherein the vector comprises a capsid derived from AAV7m8, AAV9, AAV2-retro, or AAVrh.10.

84. A cell comprising the expression construct of any one of claims 1-60 or the vector of any one of claims 61-82.

85. A pharmaceutical composition comprising (i) the expression construct of claims 1-60 or the vector of any one of claims 61-83 and (ii) a pharmaceutically acceptable carrier.

86. A method of increasing ATP7B activity in a subject in need thereof, the method comprising administering to the subject the vector of any one of claims 61-82 or the pharmaceutical composition of claim 85.

87. A method of increasing copper secretion in a subject in need thereof, the method comprising administering to the subject the vector of any one of claims 61-82 or the pharmaceutical composition of claim 85.

88. A method of treating a condition caused by a deficiency or dysfunction of ATP7B in a subject in need thereof, the method comprising administering to the subject the vector of any one of claims 61-82 or the pharmaceutical composition of claim 85.

89. A method of treating Wilson's Disease in a subject in need thereof, the method comprising administering to the subject the vector of any one of claims 61-82 or the pharmaceutical composition of claim 85.

90. A method of reducing dystonia or bradykinesia in a subject suffering from Wilson's Disease, the method comprising administering to the subject the vector of any one of claims 61-82 or the pharmaceutical composition of claim 85.

91. A method of reducing the incidence of leukopenia or anemia in a subject suffering from Wilson's Disease, the method comprising administering to the subject the vector of any one of claims 61-82 or the pharmaceutical composition of claim 85.

92. A method of reducing the incidence of cirrhosis in a subject suffering from Wilson's Disease, the method comprising administering to the subject the vector of any one of claims 61-82 or the pharmaceutical composition of claim 85.

93. The method of any one of claims 86-92, wherein the subject is a human.
